(19) United States
(12) Patent Application Publication
(10) Pub. No.: US 2013/0190379 A1
(43) Pub. Date: Jul. 25, 2013

(54) SMALL RNA MOLECULES, PRECURSORS THEREOF, MEANS AND METHODS FOR DETECTING THEM, AND USES THEREOF IN TYPING SAMPLES

(71) Applicants: Edwin Pieter Johan Cuppen, Utrecht (NL); Gerrit Albert Meijer, Hattem (NL); Eugene Berezikov, Vleuten (NL); Maria Begona Diosdado Calvo, Zeist (NL)

(72) Inventors: Edwin Pieter Johan Cuppen, Utrecht (NL); Gerrit Albert Meijer, Hattem (NL); Eugene Berezikov, Vleuten (NL); Maria Begona Diosdado Calvo, Zeist (NL)

(21) Appl. No.: 12/667,998

(22) PCT Filed: Jul. 7, 2008
(86) PCT No.: PCT/NL08/50459

§ 371 Date: Sep. 27, 2010

Related U.S. Application Data
(60) Provisional application No. 60/958,733, filed on Jul. 6, 2007.
(60) Provisional application No. 60/961,001, filed on Jul. 17, 2007.

(30) Foreign Application Priority Data
Jul. 6, 2007 (EP) 07075567.3

(51) Int. Cl. C12Q 1/68; C12N 15/113; C12N 15/10

(52) U.S. Cl. CPC C12Q 1/6886; C12N 15/1072; C12N 15/113
USPC 514/44 A; 536/24.31; 506/9; 536/24.5

(57) ABSTRACT

The invention is related to small RNA molecules, precursors thereof and methods for detecting such molecules. The invention is in particular concerned with differentially expressed small RNA molecules and precursors thereof. Various collections of small RNA molecules, precursors thereof and collections of probe and primers that can be used to detect small RNA molecules, precursors thereof are provided. Further provided are diagnostic test based on this differential expression. Also provided are therapeutic applications using one or more of the members of the presented collections.

Cancer of the colorectal part of the gastrointestinal tract is a frequently occurring disorder. In a first stage a benign tumour (adenoma) occurs which can turn into a malignant cancer (adenocarcinoma). Not all adenomas progress to carcinomas. In fact this progression into a carcinoma happens in only a small subset of tumours. Initiation of genomic instability is a crucial step and occurs in two ways in colorectal cancer (Lengauer et al. (1998) Nature, 396, 643-649). DNA mismatch repair deficiency leading to microsatellite instability (abbreviated as MSI or MIN), has been most extensively studied (di Pietro et al. (2005) Gastroenterol., 129, 1047-1059), and explains about 15% of adenoma to carcinoma progression. MSI tumours occur due to defects in the DNA mismatch repair genes (MMR), which promote tumour progression due to the accumulation of mutations and epigenetic changes. In the other 85% of cases where colorectal adenomas progress to carcinomas, genomic instability occurs at the chromosomal level (CIN) giving rise to aneuploidy. While for a long time these chromosomal aberrations have been regarded as random noise, secondary to cancer development, it has now been well established that these DNA copy number changes occur in specific patterns and are associated with different clinical behaviour (Hermsen et al. (2002). Gastroenterol. 123, 1109-1119). Chromosomal aberrations frequently reported in colorectal cancers are 7pq, 8q, 13q, 20q gains and 4pq, 5q, 8p, 15q, 17p, 18q losses. It was shown that 8q, 13q and 20q gains and 8p, 15q, 17p and 18q losses, are associated with progression of colorectal adenomas to carcinomas (Hermsen et al. (2002) cited above). In summary, colorectal cancer results from the accumulation of chromosomal aberrations, mutations and epigenetic changes ultimately causing alterations in gene expression at the RNA and protein level. Recent investigations have shown that a novel class of non-coding RNA molecules called microRNAs (miRNAs) also can act as oncogenes and tumour suppressors during cancer development. These non-coding RNAs contribute to tumour progression due to changes in their expression. Microarray based profiling studies have shown in a number of different tumor tissues and tumor cell lines, that miRNAs are differentially expressed. This differential expression, in some cases, was correlated to type of tumor and could be used to distinguish normal versus aberrant (tumor) tissue (Bloomston et al. (2007) JAMA 297:1901-1908), Iorio et al. (2005) Cancer Res. 65:7065-7070. In some cases, the unique small RNA profile correlated with prognosis (Yanaihara et al. (2006) Cancer Cell 9:189-198, Calin et al. (2005) N. Engl. J. Med. 353:1793-1801). Importantly, expression profiling studies in chronic lymphocytic leukemia have shown that miRNAs signatures can classify leukemia samples according to their developmental history and categorize poorly differentiated tumours more accurately than mRNA expression profiles (Calin et al. (2005) N. Engl. J. Med. 353:1793-1801).

Therefore, these experiments reveal that the accumulation of specific combinations of chromosomal aberrations, epigenetic changes and mutations that characterize a tumour, determine its miRNA expression signature and vice versa. Consequently, miRNA expression profiles are a powerful tool for diagnosis, determining prognosis, progression risk and response to therapy of human cancers.

It is an object of the invention to molecularly identify and characterize the above described progression of adenomas into adenocarcinomas in as early a stage as possible, to allow early stage and more specific treatment of (pre-)malignant tumors while avoiding unnecessary surgical intervention. It is a further object of the invention to identify, high-risk pre-malignant colorectal tumors at a stage when the malignant phenotype (i.e. invasion and metastastis) has not yet emerged. At such a stage there usually are no symptoms yet that evoke clinical evaluation resulting in in situ optical analysis or microscopic analysis of biopsy or resection material. Current diagnostic procedures for early detection of colorectal cancer include colonoscopy, sigmoidoscopy and fecal tests such as faecal occult blood test (FOBT) and DNA faeces tests. Colonoscopy and sigmoidoscopy are not only quite uncomfortable for the patient, but specificity is low since many non-progressive adenomas are detected. Secondly, sensitivity is hampered by experience and devotion (retraction time) of the endoscopist and shape of the tumors (flat lesions go undetected more easily than polypoid). In addition, sigmoidoscopy only covers part of the large intestine. Polypectomies, endoscopic resections or biopsies during colonoscopy are performed when colorectal tumors/lesions are identified to confirm the nature of these lesions by histological analysis. Existing commercial fecal based tests have yielded promising results but need further improvement. FOBT (Faecal Occult Blood Test) is the most recognized method of selecting high risk individuals, and has been demonstrated to reduce death from colorectal cancer at the population level when applied in a population based screening program. Unfortunately, with FOBT a considerable percentage of the tumours is missed and the already bleeding tumour is often discovered at a relatively late stage. DNA faeces tests so far have mainly focused on detecting point mutations in only a limited number of genes, i.e. KRas, APC and p53. kRas mutations have proven not to be very informative, and testing for APC and p53 mutations is technically demanding, and thus expensive. In addition, substantial numbers of CRC do not carry these mutations.

More recently, DNA microarray-based tumor gene expression profiles have been used for cancer diagnosis. However, studies have been limited to a few cancer types and have spanned multiple technology platforms complicating comparison among different datasets. Expression analysis performed at the protein level has a number of drawbacks, the first being linked to the origin of the samples, i.e. the need for an invasive colonoscopy.

The present invention shows that small RNAs (miRNAs and other small RNAs) can serve as informative biomarkers for early detection/secondary prevention of colorectal cancer and biological classification of established colorectal cancer in order to facilitate therapeutic decision making. As miRNAs and other small RNAs are produced from longer precursors it is also possible to use the precursor as a biomarker. As these RNA molecules are typically detected in groups, for instance, using (micro)arrays, the present invention also provides collection of probes for the detection of miRNAs, other small RNAs and/or precursors thereof. These collections are not only useful for the detection of said biomarkers in the context of colorectal cancer. They are also useful in other diagnostic tests to type or classify samples of nucleic acid. mRNAs and other small RNAs are oligonucleotides of between 15 and 200 bases, preferably between 15-100 bases. Typically these RNAs are between 18 and 27 bases.

The invention therefore provides a collection of probes or primers for detecting at least 5, preferably at least 10, more preferably at least 20 of the nucleic acid molecules of table 23 (the miRNA and star sequences) and/or table 24 (the pre-miRNA sequences). In a preferred embodiment said collection of probes or primers comprises probes or primers for at least 100, more preferably at least 500 of the nucleic acid molecules of table 23 (the miRNA and star sequences) and/or table 24 (the pre-miRNA sequences). Preferably said collection of probes or primers comprises at least one probe for each of the nucleic acid molecules of table 23 (the miRNA and star sequences) and/or table 24 (the pre-miRNA sequences). In a preferred embodiment said collection of probes or primers comprises probes or primers for detecting nucleic acid molecules of table 23 (the miRNA and star sequences). Collections of these probes or primers more accurately classify samples, presumable because these molecules and not the precursors are functional in a cell.

In a preferred embodiment a collection of probes of the invention comprises the nucleic acid sequences identified in table 21. These collections are particularly suited for diagnostic tests. The expression levels of nucleic acid molecules identified in table 23 or in table 24 can be determined by labelling the small RNA complement of a sample, for example by direct chemical crosslinking of label to the RNA backbone, end-labelling by adapter ligation or RNA extension, and subsequent hybridization to reverse complement DNA probes (for instance as depicted in table 21) under stringent conditions using state-of-the-art techniques. Other, potentially more sensitive methods for small RNA detection have been described, including the use of DNA-modified probes (e.g. locked-nucleic-acid LNA probes) for hybridization and nucleic acid amplification-based methods using primers specific for the nucleic acid molecules identified in table 23 or in table 24. A preferred method of amplification is real-time PCR. Means and methods for amplifying miRNA or other small RNA molecules and precursors thereof are described in Lee et al (2006) Int. J. Cancer Vol 120, pp 1046-1054; Gaur et al (2007) Cancer Res Vol. 67, pp. 2456-2468; Bandres et al (2006) Molecular Cancer Vol 5: 29). Hybridization is preferably performed under stringent conditions. Hybridization stringency regulates the percentage of nucleotides which must match on two unrelated single-stranded nucleic acid molecules before they will base pair with each other to form a duplex, given a certain set of physical and chemical conditions. The hybridization stringency is used to determine when a hybridization probe and a target nucleic acid will come together, and can be set by the researcher by varying the conditions. In general, if the percentage of matching nucleotides is lower than 90 percent, the two single-stranded nucleic acid molecules are considered nonhomologous and any hybridization is considered nonstringent. Conditions which can be used for stringent hybridization of the probes and primers of the invention are known to person skilled in the art. Other conditions can be obtained by a person skilled in the art by comparing hybridization of the probe or primer with a sequence that has more than 10 percent mismatches with the target sequence. In this way conditions can be selected that allow discrimination between the amount of hybridization to the unmodified target sequence and the sequence having 10% mismatches. Typically, though not necessarily, not all of the probes give a signal when analysing nucleic acid samples. However, starting with a collection of probes of the invention one can easily compare levels of nucleic acid of table 23 or table 24, for instance through hybridization of the respective probes and select probes for inclusion in the diagnostic test on the basis of the levels of hybridization. An obtained pattern for the levels of the nucleic acids such as the pattern obtained for the levels of hybridization to the respective probes is indicative for the type of sample tested. Different types of samples give different patterns and can thus be classified on the basis of pattern. To this end the invention provides the use of a collection of probes or primers of the invention for detecting the levels of at least 10 nucleic acid molecules of table 23 or table 24, preferably through detecting hybridization of nucleic acid from a sample to a collection of probes of the invention. The same types of samples often show a limited amount of variation with respect to the patterns they produce. It is possible to select probes or primers that produce more homogeneous patterns by testing a number of samples. To this end in a preferred embodiment the invention provides a method for selecting probes for testing nucleic acid samples said method comprising hybridizing nucleic acid from a representative number of said samples to a collection of probes according to the invention,
quantifying hybridization of said probes with nucleic acid in said representative number of samples, and
selecting a subset of probes from said collection of probes, that (characteristically) hybridizes to nucleic acid of said sample or that (characteristically) not hybridize to said nucleic acid of said sample. The characteristic pattern can comprise specifically detected and/or specifically not detected small RNAs. The selected subset of probes can be used to determine whether a test sample is likely to be of the type of sample the probes are selected for. The selected subset of probes are detectable and thus identify nucleic acid molecules of table 23 or table 24 that are present in the representative number of samples. These identified nucleic acid molecules can subsequently further be detected by a number of other methods as discussed herein above. One, non-limiting other method is real-time PCR using primers that are specific for the particular nucleic acid molecules. The likelihood of a correct typing of the sample improves when the method of selection involves two different types of samples and probes or primers are selected that detect differences in the levels of nucleic acid molecules, preferably through exhibit differential levels of hybridization between the two types of samples. Typically one type of sample is used as a reference whereas the other is of the same type as the type the test sample is to be tested. To select probes the invention provides a method for selecting a collection of probes that discriminates between at least two types of samples comprising nucleic acid from cells said method comprising
hybridizing nucleic acid from a representative number of samples of said at least two types of samples to a collection of probes according to the invention,
quantifying hybridization of said probes with nucleic acid in said samples, and
selecting a subset of probes from said collection of probes, that discriminates between said at least two types of samples. The selected subset of probes identify nucleic acid molecules of table 23 or table 24 that are differentially present in the at least two samples. These identified nucleic acid molecules can subsequently further be detected using a number of other methods as discussed herein above. One, non-limiting other method is real-time PCR using primers that are specific for the particular nucleic acid molecules. Collections of probes or primers of the invention are particularly suited for typing human cells, thus preferably said cells are human cells. They are further suited for inclusion into a test that distinguishes, types and/or discriminates between different types of samples. Diagnostic tests are preferred. Such tests are able to identify, distinguish, type and/or discriminate samples from an individual. The at least two samples can be derived from different parts of the body, however, preferably the at least two samples are from the same tissue but different in for instance, developmental stage or disease stage. The at least two samples do not have to be derived from the same body or individual. In fact, often the test sample and a reference sample are derived from different individuals. A method of selecting probes can distinguish 2 samples (when they are different). In a diagnostic test it will often not be a comparison of two samples, but merely a comparison of the expression profile (hybridization levels) of the test sample with the predefined levels found based upon the training set of the diagnostic test. These last levels will lead to a classification rule (algorithm) in a computer program and the expression levels found in a test sample will be classified according to that rule. So in practice, the actual test no longer requires a comparison between a reference and a test sample, but only the comparison with a reference.

The predictive value of classifiers as described above can be further improved. A first way would be to increase the sample set this reduces noise and leads to the selection of more predictive small RNAs. This can in some cases increase the accuracy of prediction and decrease the error rate. However, in other cases the predictive value of a diagnostic test has already reached its limit and increasing the sample set will not result in a more accurate predictive set. A further way to improve the predictive value of a diagnostic test of the invention is to combine the dataset based on the microarray data with other data sets. For example, a diagnostic test of the invention may combine data based on the small RNA expression levels with array CGH data obtained from the same samples. This can result in a more accurate prediction of disease state. A further option is to use combined diagnostic tests in a step-wise procedure, where the first diagnostic test will have the best predictive value. For some individuals that are difficult to classify with a method of the invention, a further diagnostic test will follow that will ultimately result in a diagnosis of the disease state. Where the second test on its own might not have given a clear answer, in combination with a method of the invention, a defined and reliable diagnosis can be given also for individuals or samples that are difficult to classify with high confidence with a method of the invention.

A sample can comprise cells. In a preferred embodiment the sample comprises colon cells, colon derived tumor cells or nucleic acid derived therefrom. Typically, however, a sample has undergone some type of manipulation prior to analysing the presence or absence therein of a miRNA, a small RNA and/or hairpin RNA according to the invention. Such manipulation, typically, though not necessarily comprises isolation of at least (part of) the nucleic acid of the cells. The nucleic acid in a sample may also have undergone some type of amplification and/or conversion prior to analysis with a method of the invention miRNA or a small RNA or a precursor thereof can be detected directly via complementary probe specific for said miRNA or small RNA or indirectly. Indirect forms include, but are not limited to conversion into DNA or protein and subsequent specific detection of the product of the conversion. Conversion can also involve several conversions. For instance, RNA can be converted into DNA and subsequently into RNA which in turn can be translated into protein. Of course such conversions may involve adding the appropriate signal sequences such as promoters, translation initiation sites and the like. Other non-limiting examples include amplification, with or without conversion of said miRNA or small RNA in said sample for instance by means of PCR or NASBA or other nucleic acid amplification method. All these indirect methods have in common that the converted product retains at least some of the specificity information of the original miRNA, a small RNA and/or hairpin RNA, for instance in the nucleic acid sequence or in the amino acid sequence or other sequence. Indirect methods can further comprise that nucleotides or amino acids other than occurring in nature are incorporated into the converted and/or amplified product. Such products are of course also within the scope of the invention as long as they comprise at least some of the specificity information of the original miRNA, a small RNA and/or hairpin RNA. By at least some of the specificity information of the original miRNA, a small RNA and/or hairpin RNA is meant that the converted product (or an essential part thereof) is characteristic for the miRNA, a small RNA and/or hairpin RNA of which the presence or absence is to be determined. A preferred small RNA is a miRNA. A hairpin RNA is herein also referred to as a precursor of a miRNA. The hairpin does not have to contain the entire sequence of the precursor but contains at least the stem loop structure.

A diagnostic test of the invention is particularly suited for distinguishing, typing and/or discriminating between different stages of disease. A diagnostic test of the invention preferably comprises a test that detects illness in an animal, preferably a primate, more preferably a human. A diagnostic test or a set/collection of probes of the invention is particularly suited to discriminate between different types of colon cells. A diagnostic test or a set/collection of probes of the invention are particularly suited to discriminate between samples comprising nucleic acid of normal colon cells, colon adenoma cells and/or colon carcinoma cells. A diagnostic test of the invention is particularly suited for distinguishing, typing and/or discriminating samples of different stages of tumorigenesis. Thus preferably at least one of said at least two types of samples comprises nucleic acid from tumor cells. In a particularly preferred embodiment said tumor cells are colon tumor cells. A tumor or tumour is an abnormal growth or mass of tissue. A tumor can be either malignant or benign. Preferred benign tumors are pre-malignant tumors, preferably adenomas. Nearly all tumors are examples of neoplasia, although certain developmental malformations or inflammatory masses may occasionally be referred to as tumors. This latter meaning is not intended herein. Tumors are typically caused by mutations in DNA of cells, which interfere with a cell's ability to regulate and limit cell division. Some of these mutations involve loss or gain of large parts of chromosomes (typically at least 100 megabases). Such loss or gain mutations often involve chromosome arms. An accumulation of mutations is needed for a tumor to emerge. Mutations that activate oncogenes or repress tumor suppressor genes can eventually lead to tumors. Cells have mechanisms that repair DNA and other mechanisms that cause the cell to destroy itself by apoptosis if DNA damage gets too severe. Mutations that repress the genes for these mechanisms can also eventually lead to cancer. A mutation in one oncogene or one tumor repressor gene is usually not enough for a tumor to occur. A combination of a number of mutations is necessary. As a result of the transformation tumor cells express genes and RNAs at levels that differ from their normal counterpart. The same holds true for different stages of tumorigenesis. Later stages express genes and RNAs at different levels when compared to less progressed stages. In the present invention it was further found that tumor progression can be monitored by determining the level of RNA using a collection of probes of the invention. It was found that differences in the level of RNA in samples of different stages of the same type of tumor (e.g. colorectal epithelial neoplasia) produce different signatures that can be used to predict tumor progression. Thus differences in the level of specific RNAs of a tumor at a certain stage can be used to predict progression thereof, particularly within a specific time span. The prediction is more accurate for progression within two years, particularly within a year, more in particular within 6 months from the moment the sample was taken. A diagnostic test of the invention can thus be used to determine a prognosis for the individual or the future behaviour of the tumor. A diagnostic test of the invention is particularly suited to characterise a tumor. The invention therefore provides the use of a test of the invention, preferably a diagnostic test of the invention, for characterising a tumor. A diagnostic test of the invention can also be used to predict therapy outcome or efficacy of treatment or be used to follow the effect of a therapy. The invention therefore provides the use of a test of the invention, preferably a diagnostic test of the invention for predicting therapy outcome or predicting efficacy of treatment or for following an effect of a therapy. The invention further provides a kit for a diagnostic test of the invention said kit comprising a collection of probes or primers of the invention.

A tumor is preferably a tumor of the anus, bladder, bile duct, bone, brain, breast, cervix, colon/rectum, endometrium, esophagus, eye, gallbladder, head/neck, liver, kidney, larynx, lung, mediastinum (chest), mouth, ovaries, pancreas, penis, prostate, skin, small intestine, stomach, tailbone, testicles or of the thyroid. A tumor is preferably a papilloma/carcinoma, a choriocarcinoma, an endodermal sinus tumor, a teratoma, a adenoma/adenocarcinoma, dysplasia, hyperplasia, intraepithelial neoplasia, squamous cell carcinoma, undifferentiated carcinoma, transitional cell carcinoma, carcinoma not otherwise specified, a soft tissue sarcoma, a melanoma, a fibroma/fibrosarcoma, lipoma/liposarcoma, a leiomyoma/leiomyosarcoma, a rhabdomyoma/rhabdomyosarcoma, a mesothelioma, an angioma/angiosarcoma, an osteoma/osteosarcoma, a chondroma/chondrosarcoma, a glioma or a lymphoma/leukaemia.

The invention further provides a method for selecting probes or primers of the invention for testing nucleic acid samples. Subsets of probes or primers are preferably selected from the collection of probes or primers for the nucleic acids of table 23 and/or table 24. A probe or a primer for a nucleic acid molecule of table 23 or table 24 is specific for said nucleic acid molecule when used under stringent conditions. Probes are preferably selected from the collection of probes of table 21. The selection is preferably done by comparing at least two types of samples with each other. As mentioned herein above, it is preferred that at least one of said types of samples is a particular type of tumor.

Preferably said type of sample is a particular stage of tumor type. Preferred stages are the benign stage of a tumor, the progressed but not yet metastasized stage and the metastasized stage. Thus the progressed (i.e. locally invasive) but not yet metastasized stage and the respective metastasized stages (i.e. locoregional lymph node metastasis and metastasis to distant organs). It is preferred that at least one other type of sample is a reference sample. The reference sample can be any type of sample. It is preferred that when the test type of sample is of a human, the reference sample type is also human. In one preferred embodiment said reference sample comprises normal cells, preferably from the type of cells that the tumor originated from. Normal cells, counterpart ect. are used herein typically to refer to unaffected cells, counterpart etc, such as are preferably obtained from a healthy individual. In another preferred embodiment said normal cells are obtained from an affected individual but from a site that is not affected by the disease. In a preferred embodiment said normal cells are of the same type of cells that the test sample is derived from and that are suspected of containing diseased cells. In another preferred embodiment the reference sample is the same type of tumor but at a different stage of progression. In yet another preferred embodiment the reference sample is of a different tissue, preferably from blood. In a preferred embodiment the sample and the reference (sample) is a blood sample. A preferred type of blood sample is whole blood, preferably peripheral whole blood or a fraction thereof. A preferred fraction is the fraction of peripheral blood mononuclear cells (PBMC). The small RNA or precursors do not have to be present in intact cells in the blood but can also be present in free form or associated with cell debris or in protein bound complexes. Thus in another preferred embodiment said blood sample comprises serum or plasma. Plasma and serum are particularly suited to detect a nucleic acid molecule of table 23 or table 24 that is not normally present in blood cells. A tumor sample can be obtained in a number of ways. A preferred sample is a biopsy sample. Another preferred sample is a fine needle aspirate (FNA), stool or faeces sample. Such samples contain cells and cell derivatives from which RNA can be prepared. These samples are particularly useful when the individual is suffering from or suspected of suffering from a colon tumor. The invention thus further provides method for detecting cells in a faeces sample comprising detecting small RNA or precursors therefore in said sample. The small RNA or precursors therefore do not have to be present in intact cells in faeces but can also be associated with cell debris or in protein bound complexes. The invention thus further provides method for detecting small RNA or precursors thereof in a faeces sample comprising detecting small RNA in said sample. In a preferred embodiment said method comprises detecting the level of nucleic acid molecules identified in table 8, table 9a, table 9b, table 10a, table 10b, table 11a, table 11b, table 12a, table 12b, table 12c, table 23, table 24 and/or table 34. Preferably the level of the nucleic acid molecules identified in table 11a and/or table 11b is detected. In a particularly preferred embodiment the level of the nucleic acid molecules identified in table 12a, table 12b, table 12c and/or table 34 is detected.

Many tumors, particularly at the later stages shed cells into the blood stream. These cells can be detected. Using a method for selecting probes of the invention it is possible to select probes that distinguish, type or discriminate samples of blood from healthy individuals from samples of blood from individuals that suffer from a particular type of tumor. Further provided is a method for detecting cells in a sample of blood or a fractionated part thereof, comprising detecting small RNA in said sample. The sample itself does not have to contain cells. Particularly in blood it is possible that cells can be detected that have shed the indicative nucleic acid into the blood stream, for instance upon death of said cell. Detection of this nucleic acid in the blood, plasma or serum is indicative for the cell that has shed nucleic acid in the blood stream. The cells to be detected are preferably tumor cells, more preferably colon tumor cells. In a preferred embodiment the level of nucleic acid molecules identified in table 13, table 14a, table 14b, table 15a, table 15b, table 16a, table 16b, table 18a, table 18b, or table 18c, table 23, table 24 and/or table 35 is detected. Preferably the level of the nucleic acid molecules identified in table 16a and/or table 16b is detected. In a particularly preferred embodiment the level of the nucleic acid molecules identified in table 18a, table 18b, table 18c and/or table 35 is detected.

The selection methods described herein above yield collections of probes or primers that can be used to distinguish, type and/or discriminate samples containing nucleic acid from cells. As a result the invention provides collections of probes or primers that are obtainable by such a method. In a further aspect the invention provides a subset comprising at least ten probes for the nucleic acids of table 23 and/or table 24. The invention further provides a collection of probes comprising the nucleic acid molecules identified in table 21. A subset of probes is preferably a subset of the collection of the probes of table 21. In a preferred embodiment said subset comprises at least 10 of the probes of table 21.

In a particularly preferred embodiment the invention provides a subset of probes of the collection of probes of table 21, comprising and/or consisting of the probes identified in table 1, table 2a, table 2b, table 2c, table 2d, table 3a, table 3b, table 3c, table 3d, table 4a, table 4b, table 4c, table 4d, table 5a, table 6a, table 7a, table 9a, table 9b, table 10a, table 10b, table 11a, table 11b, table 12a, table 12b, table 12c, table 14a, table 14b, table 15a, table 15b, table 16a, table 16b, table 18a, table 18b, table 18c, table 19c or table 19d, table 20, table 25a, table 26, table 27, table 28a, table 28b, table 29a, table 29b, table 30, table 31a, table 31b, table 32a, table 32b and/or table 33. In another preferred embodiment said subset of probes from the collection of probes of table 21, comprises and/or consists of the probes identified in table 8 or table 13.

In a particularly preferred embodiment the invention provides a subset of probes or primers for detecting nucleic acid molecules of table 23 or table 24, comprising and/or consisting of probes or primers for the nucleic acid molecules identified in table 1, table 2a, table 2b, table 2c, table 2d, table 3a, table 3b, table 3c, table 3d, table 4a, table 4b, table 4c, table 4d, table 5a, table 6a, table 7a, table 9a, table 9b, table 10a, table 10b, table 11a, table 11b, table 12a, table 12b, table 12c, table 14a, table 14b, table 15a, table 15b, table 16a, table 16b, table 18a, table 18b, table 18c, table 19c, table 19d, table 20, table 25a, table 26, table 27, table 28a, table 28b, table 29a, table 29b, table 30, table 31a, table 31b, table 32a, table 32b, table 33, table 34 and/or table 35. In another preferred embodiment said subset of probes or primers for detecting nucleic acid molecules of table 23 or table 24, comprises and/or consists of the probes or primers for the nucleic acid molecules identified in table 8 or table 13.

The invention further provides a method for determining whether a sample comprises nucleic acid from a particular type of cells said method comprising hybridising nucleic acid from said sample to a collection of probes for the nucleic acid molecules as identified in table 1, table 2a, table 2b, table 2c, table 2d, table 3a, table 3b, table 3c, table 3d, table 4a, table 4b, table 4c, table 4d, table 5a, table 6a, table 7a, table 9a, table 9b, table 10a, table 10b, table 11a, table 11b, table 12a, table 12b, table 12c, table 14a, table 14b, table 15a, table 15b, table 16a, table 16b, table 18, table 19c, table 19d, table 20, table 21, table 25a, table 26, table 27, table 28a, table 28b, table 29a, table 29b, table 30, table 31a, table 31b, table 32a, table 32b, table 33, table 34 and/or table 35, quantifying hybridization of said probes with nucleic acid in said samples, and comparing the result of said quantification with a reference. In a preferred embodiment said cells are tumor cells or suspected of being tumor cells. Preferably said cells are colon cells.

The invention further provides a subset of probes or primers according to the invention, comprising probes or primers for each of the nucleic acids identified in table 1, table 2a, table 2b, table 2c, table 2d, table 3a, table 3b, table 3c, table 3d, table 4a, table 4b, table 4c, table 4d, table 5a, table 6a, table 7a, table 9a, table 9b, table 10a, table 10b, table 11a, table 11b, table 12a, table 12b, table 12c, table 14a, table 14b, table 15a, table 15b, table 16a, table 16b, table 18a, table 18b, table 18c, table 19c, table 19d, table 20, table 20, table 21, table 25a, table 26, table 27, table 28a, table 28b, table 29a, table 29b, table 30, table 31a, table 31b, table 32a, table 32b, table 33, table 34 and/or table 35. Further provided is a subset of probes or primers from the collection of probes or primers of the invention, comprising probes or primers for each of the nucleic acids identified in table 8 or table 13. In a preferred embodiment said cells are tumor cells or suspected of being tumor cells. Preferably wherein said cells are colon cells.

A nucleic acid molecule of the invention such as a probe or primer often can contain a nucleotide analogue. Many analogues are presently available that mimic at least some of the base pairing characteristics of the “standard” nucleotides A, C, G, T and U. Alternatively, nucleotide analogues such as inosine can be incorporated into a nucleic acid molecule of the invention. Other types for analogues include LNA, PNA, morpholino and the like. Further methods for the specific detection of nucleic acid include but are not limited to specific nucleic acid amplification methods such as polymerase chain reaction (PCR) and NASBA. Such amplification methods typically use one or more specific primers. A primer or probe preferably comprises between 12-40 nucleotides having at least 90% sequence identity to a sequence as depicted in table 23 or table 24, or the complement thereof. A probe, primer or nucleic acid molecule of the invention is preferably single stranded. However, for shipping, production and or therapeutic purposes a double stranded molecule is sometimes preferred. For example a miRNA or small RNA is processed in the cell in double stranded form. Such analogues are considered to be a functional equivalent of a nucleic acid molecule such as a probe or a primer of the invention, when they exhibit the same hybridization characteristics under stringent conditions in kind, not necessarily in amount.

The invention further provides a method for determining whether a sample comprises nucleic acid from colorectal cells said method comprising detecting and quantifying the level of the nucleic acid molecules identified in table 1 in said sample, and comparing the result of said quantification with a reference.

Further provided is a method for determining whether a sample comprises nucleic acid from adenoma cells said method comprising detecting and quantifying the level of the nucleic acid molecules identified in table 2a, table 2b, table 2c, table 2d, table 4a, table 4b, table 4c, table 4d, table 5a, table 7a, table 9a, table 9b, table 11a, table 11b, table 12a, table 12c, table 14a, table 14b, table 16a, table 16b, table 18a, table 18c, table 19c, table 19d, table 20, table 25a, table 26, table 27, table 34 and/or table 35 in said sample, and comparing the result of said quantification with a reference. In this embodiment it is preferred that said reference is a quantification of the levels of the same nucleic acid molecules for a sample comprising nucleic acid from adenocarcinoma cells or from normal cells.

The invention further provides a method for determining whether a sample comprises nucleic acid from adenocarcinoma cells said method comprising detecting and quantifying the level of the nucleic acid molecules identified in table 3a, table 3b, table 3c, table 3d, table 4a, table 4b, table 4c, table 4d, table 6a, table 7a, table 10a, table 10b, table 11a, table 11b, table 12b, table 12c, table 15a, table 15b, table 16a, table 16b, table 18b, table 18c, table 19c, table 19d, table 25a, table 26, table 27, table 28a, table 28b, table 29a, table 29b, table 30, table 31a, table 31b, table 32a, table 32b, table 33, table 34 and/or table 35 in said sample, and comparing the result of said quantification with a reference. In this embodiment it is preferred that the level of the nucleic acid molecules identified in table 4a, table 4b, table 4c, table 4d, table 7a, table 11a, table 11b, table 12c, table 16a, table 16b, table 18c, table 19c, table 19d, table 25a, table 26, table 27, table 28a, table 28b, table 29a, table 29b, table 30, table 31a, table 31b, table 32a, table 32b, table 33, table 34 and/or table 35 is quantified. In a particularly preferred embodiment the level of the nucleic acid molecules identified in table 4c, table 4d, table 7a, table 25a and/or table 26 is quantified. In this embodiment it is preferred that said reference is a quantification of the levels of the same nucleic acid molecules for a sample comprising nucleic acid from adenoma cells or from normal cells. In a preferred embodiment said normal cells are brushed cells from normal colon mucosa.

In one aspect the invention provides a method for determining whether a sample comprises nucleic acid from colorectal cells said method comprising hybridising nucleic acid from said sample to a collection of probes as identified in table 1, quantifying hybridization of said probes with nucleic acid in said samples, and comparing the result of said quantification with a reference. In another aspect the invention provides a method for determining whether a sample comprises nucleic acid from adenoma cells said method comprising hybridising nucleic acid from said sample to a collection of probes as identified in table 2a, table 2b, table 2c, table 2d, table 4a, table 4b, table 4c, table 4d, table 5a, table 7a, table 9a, table 9b, table 11a, table 11b, table 12 a and c, table 14a, table 14b, table 16a, table 16b, table 18a and c, table 19c, table 19d, table 20, table 25a, table 26, table 27, table 34 and/or table 35, quantifying hybridization of said probes with nucleic acid in said sample, and comparing the result of said quantification with a reference. In a preferred embodiment said reference is a quantification of a hybridization of a reference sample with said collection of probes. Preferably said reference sample comprises nucleic acid from adenocarcinoma cells. In this preferred embodiment it is preferred that said collection of probes is a collection of probes as identified in table 4a, table 4b, table 4c, table 4d, table 7a, table 11a, table 11b, table 12c, table 16a, table 16b, table 18c, table 19c, table 19d, table 20, table 21, table 25a, table 26, table 27, table 34 and/or table 35. In another preferred embodiment said reference sample comprises nucleic acid from normal cells. In this preferred embodiment it is preferred that said collection of probes is a collection of probes as identified table 2a, table 2b, table 2c, table 2d, table 5a, table 9a, table 9b, table 12a, table 14a, table 14b, table 18a, table 20 and/or table 21.

In another aspect the invention provides a method for determining whether a sample comprises nucleic acid from adenocarcinoma cells said method comprising hybridising nucleic acid from said sample to a collection of probes as identified in table 3a, table 3b, table 3c, table 3d, table 4a, table 4b, table 4c, table 4d, table 6a, table 7a, table 10a, table 10b, table 11a, table 11b, table 12b and c, table 15a, table 15b, table 16a, table 16b, table 18b and c, table 19c, table 19d, table 21, table 25a, table 26, table 27, table 28a, table 28b, table 29a, table 29b, table 30, table 31a, table 31b, table 32a, table 32b, table 33, table 34 and/or table 35, quantifying hybridization of said probes with nucleic acid in said sample, and comparing the result of said quantification with a reference. In a preferred embodiment said reference is a sample comprising nucleic acid from adenoma cells. In this preferred embodiment it is preferred that said collection of probes is a collection of probes as identified in table 4a, table 4b, table 4c, table 4d, table 7a, table 11a, table 11b, table 12c, table 16a, table 16b, table 18c, table 19c, table 19d, table 21, table 25a, table 26, table 27, table 28a, table 28b, table 29a, table 29b, table 30, table 31a, table 31b, table 32a, table 32b, table 33, table 34 and/or table 35. In another preferred embodiment said reference is a sample comprising nucleic acid from normal cells. In this preferred embodiment it is preferred that said collection of probes is a collection of probes as identified in table 3a, table 3b, table 3c, table 3d, table 6a, table 10a, table 10b, table 12b, table 15a, table 15b, table 18b, table 19c, table 19d and/or 21. In a preferred embodiment said normal cells are blood cells or brush cells from normal colon.

It has been found that probes or primers for miRNAs, small RNAs and/or their precursors are particularly suited for detecting cells that have a chromosomal aberration. The invention thus further provides the use of a collection of probes or primers for miRNAs, small RNAs or their precursors, for classifying cells as cells that contain a chromosomal aberration. These aberrations typically involve a significant part of a chromosome. The aberrations can be the direct origin of the miRNA or small RNA that is being tested for. Alternatively, as miRNA and small RNA regulate the expression of other transcription units and are themselves derived from transcription, the aberration can be detected through its action on the levels of particular miRNA, small RNA or the precursor thereof in a cell. This aspect of the invention is particularly suited for detecting tumor cells that have chromosomal aberrations, and consequently are prone to progression from a premalignant to malignant stage, or have already done so. Preferably said tumor cells are colon tumor cells. This aspect can be used to detect aberrations involving segments of DNA in a chromosome that encode one or multiple small RNAs. In principle it is possible to detect even smaller aberrations. A particularly preferred type of aberration is the loss or gain of a part of a chromosome, preferably a gain of 7pq, 8q, 13q or 20q or a loss of 4pq, 5q, 8p 15q, 17p or 18q, particularly preferred is a loss of chromosome 8p or 17p. In a preferred embodiment said loss of a chromosome 8p or 17p is detected using at least one probe or primer for nucleic acid molecules of table 23 or table 24, as identified in table 19c (for loss of 8p) or table 19d (for loss of 17p). Preferably said loss of a chromosome 8p or 17p is detected using at least 5, more preferably at least 10 and most preferred all of the probes or primers for nucleic acid molecules of table 23 or table 24, as identified in table 19c (for loss of 8p) or table 19d (for loss of 17p).

A sample can be any type of sample as long as it contains nucleic acid. It is preferred that said sample contains complex nucleic acid, preferably nucleic acid derived from a cell. A collection of probes of the invention can be used to analyze any type of nucleic acid, be it DNA, RNA or an analogue thereof having the same hybridization characteristics in kind not necessarily in amount. In a preferred embodiment said nucleic acid comprises RNA derived from cells. This aspect of the invention is useful for determining levels of different RNAs in the cells. In a particularly preferred embodiment the sample is enriched for RNA, preferably for RNA that is smaller than 500 nucleotides. In a particularly preferred embodiment said sample is enriched for RNA that is about 200 nucleotides. Thus in a preferred embodiment a collection of probes or primers of the invention, or a method of the invention involving detecting of a nucleic acid molecule of table 23 or table 24, is used to detect RNA in a sample, preferably small RNA.

miRNAs and other small RNAs are found to be relevant for the tumorigenic state of a tumor. The invention provides miRNA precursors thereof that are up or down regulated in a tumor when compared to normal cells, or up or down regulated in a progressed form of said tumor when compared to the less progressed state. It is thus possible to counteract this level by increasing the level when it is down regulated or decreasing the level when it is upregulated. In this way it is possible to intervene with the tumor or the tumor state. Thus the invention further provides a method for the treatment and/or prevention of a disease in an individual comprising administering to said individual at least one nucleic acid molecule of table 23 or table 24, at least one nucleic acid molecule comprising the reverse complementary sequence of a nucleic acid molecule of table 23 or table 24, or a functional equivalent of said at least one nucleic acid molecule. Preferably said individual is suffering from colon cancer or is at risk of suffering therefrom. Said at least one nucleic acid molecule of table 23 or table 24 or the reverse complement thereof, is preferably a nucleic acid molecule of table 2a, table 2b, table 2c, table 2d, table 3a, table 3b, table 3c, table 3d, table 4a, table 4b, table 4c, table 4d, table 5a, table 6a, table 7a, table 9a, table 9b, table 10a, table 10b, table 11a, table 11b, table 12a, table 12b, table 12c, table 14a, table 14b, table 15a, table 15b, table 16a, table 16b, table 18a, table 18b, table 18c, table 19c, table 19d or table 20, table 25a, table 26, table 27, table 34 and/or table 35, or a functional equivalent thereof. For the treatment of disease, it is preferred that said at least one nucleic acid molecule of table 23 or table 24 or the reverse complement thereof, is a nucleic acid molecule of table 3a, table 3b, table 3c, table 3d, table 4a, table 4b, table 4c, table 4d, table 6a, table 7a, table 10a, table 10b, table 11a, table 11b, table 12b, table 12c, table 15a, table 15b, table 16a, table 16b, table 18b, table 18c, table 19c, table 19d, table 20, table 25a, table 26, table 27, table 34 and/or table 35 or a functional equivalent thereof. For treatment it is particularly preferred that said at least one nucleic acid molecule of table 23 or table 24 or the reverse complement thereof, is a nucleic acid molecule of table 3a, table 3b, table 3c, table 3d, table 4a, table 4b, table 4c, table 4d, table 19c, table 19d, table 20, table 26, table 27 and/or table 33, or a functional equivalent thereof. In a particularly preferred embodiment it is preferred that said at least one nucleic acid molecule of table 23 or table 24 or the reverse complement thereof is a nucleic acid molecule of table 4c, table 4d, table 19c, table 26, table 27 and/or table 33. Using at least one of these nucleic acid molecules for the treatment it is possible to modify cells of a progressed tumor (preferably an adenocarcinoma tumor) such that they become less progressed (preferably resembling a more adenoma phenotype). For the prevention of disease, it is preferred that said at least one nucleic acid molecule of table 23 or table 24 or the reverse complement thereof, is a nucleic acid molecule of table 2a, table 2b, table 2c, table 2d, table 4a, table 4b, table 4c, table 4d, table 5a, table 7a, table 9a, table 9b, table 11a, table 11b, table 12a, table 12c, table 14a, table 14b, table 16a, table 16b, table 18a, table 18c, table 19c, table 19d, table 20, table 25a, table 26, table 27, table 34 and/or table 35 or a functional equivalent thereof. For prevention it is preferred that at least one nucleic acid molecule of table 23 or table 24 or the reverse complement thereof, comprises a nucleic acid molecule of table 2a, table 2b, table 2c, table 2d, table 4a, table 5a, table 7a, table 20, table 25a, table 26 and/or table 27 or a functional equivalent thereof. Using at least one nucleic acid molecule for prevention of disease it is possible to modify cells of a benign stage such that the propensity for tumor progression is reduced in these cells.

In case of a nucleic acid molecule that is downregulated it is preferred that said nucleic acid molecule is provided to the cell wherein it is down-regulated. To this end it is preferred that said at least one nucleic acid molecule of table 23 or table 24, comprises a nucleic acid molecule of table 2d, table 3d, table 4d, table 19c, table 19d, table 27 or table 33. In a preferred embodiment said at least one nucleic acid molecule of table 23 or table 24, comprises a nucleic acid molecule of table 2d, table 3d, table 4d, or table 27 or a functional equivalent thereof. These preferred nucleic acid molecules are highly correlated with the tumor or tumor stage and or thus effective in a larger fraction of the tumors (stage).

In case of a nucleic acid molecule that is up regulated it is preferred that said nucleic acid molecule is reduced in the cell wherein it is up regulated. To this end it is preferred that said at least one nucleic acid molecule comprising the reverse complementary sequence of a nucleic acid molecule of table 23 or table 24, comprises a nucleic acid molecule of table 2c, table 3c, table 4c or table 19c or table 19d. Preferably said at least one nucleic acid molecule of table 23 or table 24, comprises a nucleic acid molecule of table 2c, table 3c or table 4c, or a functional equivalent thereof. These preferred nucleic acid molecules are highly correlated with the tumor or tumor stage and or thus effective in a larger fraction of the tumors (stage).

In one embodiment the invention provides a pharmaceutical composition, comprising as an active agent at least one nucleic acid molecule of table 23a or table 24a, or a nucleic acid molecule comprising the reverse complement of at least one nucleic acid molecule of table 23a or table 24a or a functional equivalent of such nucleic acid molecules, and optionally a pharmaceutically acceptable carrier. A pharmaceutical composition according to the invention further optionally comprises another additive. Such another additive can for example be a preservative or a colorant. Alternatively an additive is a known pharmaceutically active compound. A carrier is any suitable pharmaceutical carrier. A preferred carrier is a compound that is capable of increasing the efficacy of a nucleic acid molecule to enter a target-cell. Examples of such compounds are liposomes, particularly cationic liposomes. A composition is for example a tablet, an ointment or a cream. Preferably a composition is an injectable solution or an injectable suspension. In one embodiment the invention provides a pharmaceutical composition according to the invention for diagnostic applications. In another embodiment the invention provides a pharmaceutical composition according to the invention for therapeutic applications. In a preferred embodiment the invention provides a pharmaceutical composition according to the invention, as a modulator for a developmental or pathogenic disorder. In a preferred embodiment said developmental or pathogenic disorder is cancer. A miRNA molecule for example functions as a suppressor gene or as a regulator of translation of a gene. A method for the treatment of an individual to obtain therein a described effect, wherein said individual is provided with a small RNA, precursor thereof, or the reverse complement thereof as described herein, is similarly a use of said small RNA, precursor thereof, or the reverse complement for the preparation of a medicament for obtaining said described effect. A pharmaceutical preferably comprises a pharmaceutically acceptable carrier or excipient.

A functional equivalent of a nucleic acid molecule of table 23a, for the purpose as a pharmaceutical, is preferably a nucleic acid comprising at least 12 and preferably at least 18 consecutive bases of the nucleic acid molecule of table 23a. The functional equivalent nucleic acid may have 1 or 2, preferably only 1 mismatch with the nucleic acid of table 23a. Preferably there are no mismatches in said consecutive stretch.

A functional equivalent of a nucleic acid of table 24a is a nucleic acid having at least 90%, and preferably at least 95%, more preferably at least 99% sequence identity to a nucleic acid of table 24a. The sequence of the small RNA therein is preferably a nucleic acid comprising at least 12 and preferably at least 18 consecutive bases of the small RNA nucleic acid molecule of table 23a. The corresponding small RNA part preferably comprises no more than 2 nucleic acid mismatches. In a particularly preferred embodiment the sequence of the small RNA in said functional equivalent is exactly the same as the sequence thereof given in table 23a.

A functional equivalent of a nucleic acid molecule comprising the reverse complement a nucleic acid molecule of table 23a is preferably a nucleic acid comprising the reverse complementary sequence of at least 12 and preferably at least 18 consecutive bases of the nucleic acid molecule of table 23a. The functional equivalent nucleic acid may have 1 or 2, preferably only 1 mismatch with the reverse complement nucleic acid of table 23a. Preferably there are no mismatches in said consecutive complementary stretch.

A functional equivalent of the reverse complement a nucleic acid molecule of table 24 is a nucleic acid of which its reverse complement has at least 90%, and preferably at least 95%, more preferably at least 99% sequence identity to a nucleic acid of table 24a.

A nucleic acid molecule for use as a pharmaceutical is preferably a nucleic acid molecule as identified in table 2d, 3d, 4d, 19c, 19d, 28a, table 28b, table 30, table 32a, table 32b, or 33. Preferably said nucleic acid molecule is also identified in table 19c, table 19d, table 20, table 26 and/or table 27.

A nucleic acid molecule that comprises a reverse complement of a nucleic acid of table 23a is preferably a nucleic acid molecule as identified in table 2c, table 3c, table 4c, table 31a or table 31b. Preferably said nucleic acid molecule is also identified in table 20, table 26 and/or table 27.

A nucleic acid molecule for use as a pharmaceutical as described herein can be an RNA molecule, a DNA molecule, a hybrid thereof or other be modified. For instance, many variants of the naturally occurring bases are known and many more will likely be developed. These synthetic bases can share the same hydrogen bonding characteristics with it natural counterpart. These synthetic bases can be incorporated into a nucleic acid of the invention. A nucleic acid molecule of the invention therefore can include one or more of such synthetic bases. A nucleic acid molecule of the invention can be modified to obtain a desired effect. Examples of nucleic acid molecules comprising such modifications are locked nucleic acid modifications (LNA), morpholino modifications and other modifications. Furthermore, the backbone can be modified to provide resistance to RNAseH. A nucleic acid molecule of the invention may comprise modifications to obtain a variety of effects. For instance, the nucleic acid molecule may be modified to enhance stability, hybridization characteristics, cellular uptake, circulation half life, and targeting of the nucleic acid.

A nucleic acid molecule according to the invention is administered by any suitable known method. The mode of administration of a pharmaceutical composition of course depends on its form. In a preferred embodiment a solution is injected in a tissue. A nucleic acid molecule according to the invention is introduced in a target cell by any known method in vitro or in vivo. Said introduction is for example established by a gene transfer technique known to the person skilled in the art, such as electroporation, microinjection, DEAE-dextran, calcium phosphate, cationic liposomes or viral methods.

A nucleic acid of the invention, such as a small RNA, a miRNA, a precursor thereof or the reverse complement of the mentioned molecules can be administered directly to a cell or an individual, or through a gene delivery vehicle. The gene delivery vehicle preferably comprises an expression cassette containing a promoter and other transcription regulatory and signaling sequences to express the small RNA, a miRNA, a precursor thereof or the reverse complement thereof. The gene delivery vehicle is preferably a viral vector or a plasmid.

The invention further provides the use of a nucleic acid molecule of table 23 or table 24, as a drug target. Preferably provided is the use of a nucleic acid molecule of table 3a, table 4a, table 20, table 26, table 27 or table 33, as a drug target. Also provided is the use of a nucleic acid molecule of table 23 or table 24 or the reverse complement thereof, for identifying a molecular process in a cell that is contributing to a disease or a disease state. Preferably provided is the use of a nucleic acid molecule of table 3a, table 4a, table 20, table 26, table 27 or table 33, for identifying a molecular process in a cell that is contributing to a disease or a disease state.

In yet another embodiment the invention provides a collection of nucleic acid molecules comprising the nucleic acid molecules of table 23 or table 24. Also provided is a subset of this collection of nucleic acids comprising at least 10 of the nucleic acids identified in table 23 or table 24. In a preferred embodiment said subset comprises the nucleic acid molecules identified in table 1, table 2a, table 2b, table 2c, table 2d, table 3a, table 3b, table 3c, table 3d, table 4a, table 4b, table 4c, table 4d, table 5a, table 6a, table 7a, table 9a, table 9b, table 10a, table 10b, table 11a, table 11b, table 12a, table 12b, table 12c, table 14a, table 14b, table 15a, table 15b, table 16a, table 16b, table 18a, table 18b, table 18c, table 19c, table 19d, table 20, table 25a, table 26, table 27, table 28a, table 28b, table 29a, table 29b, table 30, table 31a, table 31b, table 32a, table 32b, table 33, table 34 and/or table 35. In a particularly preferred embodiment said subset comprises the nucleic acid molecules identified in table 2d, table 3d, table 4d, table 26, table 27 and/or table 33. In another preferred embodiment said subset comprises the nucleic acid molecules identified in table 8 or table 13.

A probe or primer of the invention can be longer than the region of complementarity with the sequence of table 23 or table 24. For a probe, the reverse complement of the sequence depicted in table 23 or table 24 is preferably incorporated into another nucleic acid. This separates the probe sequence from a surface to which it is attached. Such a probe typically exhibits improved hybridization characteristics. A probe or primer of the invention contains at least 7 consecutive nucleotides that together are the reverse complement of a sequence of the same length in the nucleic acid molecule of table 23 or table 24. Preferably said probe or primer contains at least 12, more preferably at least 16 of such consecutive nucleotides. In a particularly preferred embodiment said probe comprises the reverse complement of the entire sequence given for the nucleic acid molecule of table 23 or table 24. A probe or primer specific for a nucleic acid molecule of table 23 may have 1 or 2, preferably only 1 mismatch (in the consecutive stretch of complementarity) with the nucleic acid of table 23 it is specific for. Preferably there are no mismatches in said consecutive stretch.

A probe or primer for that is specific for a nucleic acid of table 24 comprises at least 90%, and preferably at least 95%, more preferably at least 99% sequence identity to the reverse complement of the nucleic acid comprising the region of complementarity of the corresponding nucleic acid of table 24. A primer is typically though not necessarily between 10-30 nucleotides long. A primer can contain one or more nucleotides in addition to the region of complementarity. These sequences can, for instance, be used to attach a label.

A collection of probes of the invention is preferably associated or physically linked to a solid surface. The solid surface is preferably a microarray. In another preferred embodiment a collection of probes is associated or physically linked to a collection of beads. Preferably, the individual probe are associated or physically linked to different beads such that each bead contains essentially one probe.

A collection of probes or primers of the invention preferably comprises probes or primers specific for nucleic acid molecules of table 23a or table 24a. A collection of the invention, of course preferably contains different nucleic acid molecules. When herein a collection is said to have at least a certain number of nucleic acid molecules given in the tables, or probes or primers specific therefore, it is preferred that at least said certain number of these nucleic acid molecules, probes or primers is different. In addition to this number of different ones there may be additional identical ones. A collection of probes or primers or a subset thereof of the invention are preferably used for the detection of one or more nucleic acid molecules of table 23 or table 24. However, such collections can also serve other purposes, for instance therapeutic purposes. When herein reference is made to a collection of probes or primers of the invention, this typically also includes subsets of collection of probes and primers. The same holds true for collections of nucleic acid molecules or the reverse complement thereof.

The invention further provides a collection of nucleic acid molecules comprising the reverse complement of the nucleic acid molecules of table 23 or table 24. A non-limiting use of such collection is a collection of probes for the molecules of table 23 or table 24.

A collection of probes or a subset of probes of the invention preferably comprises no more than 15.000 different probes, preferably no more than 12.000 probes, preferably no more than 10.000 and more preferably no more than 7.000 probes, particularly for probes specific for nucleic acid molecules of table 23a or table 24a. Said preferred maximum amounts are increased by 1000 when also the probes specific for nucleic acid molecules of table 23b or table 24b are included. In a particularly preferred embodiment the collection of probes comprises a maximum of 5000 probes, preferably a maximum of 4000 probes, preferably a maximum of 3000 probes, preferably a maximum of 2000 probes, preferably a maximum of 1000 probes, preferably a maximum of 500 probes. A subset of probes or the invention is preferably present in an array. Preferably a micro-array. Preferably a collection of proves of the invention is associated with a solid surface.

In a preferred aspect the invention provides a collection of probes or primers for detecting each of the nucleic acid molecules of table 25a, table 5a and/or 6a. These collections are particularly suited to discriminate colorectal adenoma cells from normal colon cells (table 5a), colorectal carcinoma cells from normal colon cells (table 6a), and colorectal adenoma cells from colorectal carcinomal cells (table 25a). In a preferred embodiment said collection of probes or primer comprises probes or primers for detecting each of the nucleic acid molecules of table 25a and table 5a. Particularly preferred is a collection of probes or primers, comprising probes or primers for detecting each of the nucleic acid molecules of table 25a, table 5a and table 6a. Preferably said collections of probes or primers are used for discriminating between normal colorectal tissue, colorectal adenoma and colorectal adenocarcinoma.

In the present invention it was found that the small RNA of table 23 or the precursors thereof of table 24 are particularly suited to map micro alterations in the chromosomal DNA. It was found that colon carcinoma contain various characteristic micro-deletions and micro-gains (amplifications) of chromosomal DNA. Micro in this context means deletions or gains of less than 3 megabases of chromosomal DNA. Several locations were identified in colon carcinoma cells that are subject micro-deletions and/or microgains. The invention thus further provides a method for determining whether a colon cell comprises a deletion of less than 3 megabases in chromosomal region 1p32.2, 1q21.1, 1q22, 6p22.2, 7p15.2, 11p15.5, 16q12.2 or 17p 13.1 comprising determining whether said cell comprises a deletion of DNA coding for a small RNA as identified in table 28a, or a precursor thereof. Said microdeletions can be detected in various ways including PCR and other amplification strategies using primers that are specific for the deletion or the boundaries thereof. Preferably said method comprises the step that nucleic acid of said cell is contacted with a primer or a probe specific for said small RNA, a precursor thereof, a DNA encoding said small RNA and/or a precursor thereof. Preferably said small RNA is a small RNA as identified in table 28b. A method of this embodiment is particularly suited for typing a cell as a colon carcinoma cell. Thus a colon cell comprising a deletion as indicated herein above is a colon carcinoma cell. Thus a method of this embodiment preferably further comprises typing said cell as a colon carcinoma cell. Preferably said small RNA is a small RNA as identified in table 30. The micro-deletion characterised by the small RNAs of table 30 are characteristic for microsatellite stable colon carcinoma cells. A method of this embodiment thus preferably further comprises typing said cell as a microsatellite stable colon carcinoma cell.

In another preferred embodiment said small RNA is a small RNA as identified in table 32a or table 32b. Micro-deletions characterised by the absence of these small RNA or the corresponding precursors characterise microsatellite instable colon carcinoma cells. Preferably said small RNA is a small RNA as identified in table 32b. A method of this embodiment thus preferably further comprises typing said (colon) cell as a microsatellite instable colon carcinoma cell.

The invention further provides a method for determining whether a colon cell comprises an additional chromosomal copy of less than 3 megabases of chromosomal region 5q31.3, 8p21.3, 9p21.3, 10p15.1 or 16q13 comprising determining whether said cell comprises an additional copy of DNA coding for a small RNA as identified in table 29a, or a precursor thereof. Said microgains can be detected in various ways including PCR and other amplification strategies using primers that are specific for the amplified regions or the boundaries thereof. Preferably said method comprises a step wherein nucleic acid of said cell is contacted with a primer or a probe specific for said small RNA, a precursor thereof, a DNA encoding said small RNA and/or a precursor thereof. In a preferred embodiment said small RNA is a small RNA as identified in table 29b. Colon cells containing such microgains are colon carcinoma cells. Thus a method of the invention preferably further comprises typing said cell as a colon carcinoma cell. In a preferred embodiment said small RNA is a small RNA as identified in table 31a or table 31b. Preferably a small RNA is a small RNA as identified in table 31b. Microgains characterised by gains of these latter small RNA or precursors thereof are characteristic for microsatellite stable colon carcinoma cells. Thus preferably said embodiment further comprises typing said cell as a microsatellite stable colon carcinoma cell.

The invention further provides a method for the treatment of an individual suffering from a colon tumor comprising administering to said individual a nucleic acid comprising a nucleic acid as identified in table 28a, table 28b, table 30, table 32a, table 32b, or 33 or the precursor of said nucleic acid, or the reverse complement of a nucleic acid as identified in table 31b or a functional equivalent of said nucleic acid. By administering this nucleic acid a small RNA that is present in said (micro)deletion (table 28, table 30, table 32 and table 33) is provided to the colon carcinoma cell thereby supplementing the reduced level of said small RNA in said cell and thereby reducing a tumorigenic state thereof. Preferably said nucleic acid is a nucleic acid as identified in table 28b, table 32b or table 33.

By administering the reverse complement of a small RNA that is present in said microgain (table 31), the colon carcinoma cell is provided with a means for reducing the expression of the overexpressed therein. This reduces the tumorogenic state of said colon carcinoma cell. Preferably said nucleic acid comprises the reverse complement of a small RNA of table 31. Preferably the reverse complement of a small RNA of table 31b.

In a preferred embodiment the nucleic acid is provided as a nucleic acid encoding a precursor for the nucleic acid. The nucleic acid may be administered as indicated herein above. In brief, it may be administered as such and/or in the context of a gene delivery vehicle.

### EXAMPLES

### Example 1

### Materials and Methods

### Samples

67 snap frozen human colorectal tumours (36 non-progressed adenomas and 31 adenocarcinomas) and ten samples of human normal colorectal epithelium obtained from histopathologically tumor free resection margins of colorectal cancer resection specimens were collected prospectively at the VU-University Medical Center (VUmc), Amsterdam, The Netherlands (Table 22). In addition, stool and blood samples from two other patients with colorectal cancer were collected. All samples were used in compliance with the institution's ethical regulations.

### Total RNA Isolation

RNA isolation was done using TRIzol and following the manufacture's protocol. Briefly, tumours samples were homologized in 2 ml Trizol using a homogenizer. 0.4 ml chloroform was added to each sample and the tube was shaken vigorously. After 3 min of incubation at room temperature, the samples were centrifuged for 10 min at 12,000 rpm at 4° C. The upper aqueous phase was transferred to a new tube and 1 ml of isopropanol was added. Then the samples were incubated at room temperature for 10 min and centrifuged for 10 min at 12,000 rpm at 4° C. The supernatant was removed by decanting and the pellets were washed by adding 1 ml of 70% ethanol and centrifuging for 5 min at 7,500 rpm at 4° C. After the 70% ethanol was removed by decanting, the pellet was briefly spun down to remove the remaining ethanol using a sharp tip. Last, the pellet was dissolved in 100 μl RNAse-free water. Total RNA was cleanup performed using the RNeasy Mini Kit (QIAgen) and according to the manufacture's protocol. Briefly, a lysis buffer (RLT) was added and mixed well. After adding 100% ethanol the samples were transferred to an RNeasy Mini spin column and centrifuging for 15 s at >10,000 rpm. The flow-through was discarded and washing of the spin column was done twice with the RPE Buffer by centrifuging for 15 s (first washing) and 2 min (second washing) at >10,000 rpm. RNA was finally eluted in a new 1.5 ml tube in 2× 30 ul of RNase-free water by centrifuging for 1 min at >10,000 rpm and stored at −80° C. until use. The quantity of the RNA samples was measured using a spectrophotometer and the quality was evaluated in by visual inspection on an agarose gel (1%).

### RNA Labeling and Isolation of Fraction of Small RNA (<100 nt)

RNA labeling and small RNA enrichement was done according Kreatech Biotechnologies' protocol (Kreatech, Amsterdam, The Netherlands). Labelled total RNA was prepared using 500 ng of total RNA as starting material, 0.5 μl of Cy3-ULS/Cy5-ULS, 0.33 μl of 10× labeling buffer solution and water to a final volume of 3.3 μl. The mix was incubated 15 min at 85° C. and then placed on ice. Clean-up of the samples was carried out using KREApure columns. Columns were prepared for use by resuspending the column material using a vortex, inserting the column in a 2 ml collection tube and centrifuging 1 min at 20,800 g. Then the flow-through was discarded and the column was rewashed by adding 300 μl of water and centrifuging 1 min at 20,800 g. After cleaning, the column was placed into a new 1.5 ml tube and the ULS-labelled total material was pipetted onto the column and centrifuged 1.5 min at 20.800 g. The flow-through containing the labeled total RNA was placed on ice and 1 μl was taken to determine the degree of labelling by measuring the OD260 and OD550 (for Cy3-ULS) or OD650 (for Cy3-ULS). After labeling, the material was subjected to small RNA isolation by adding RNAses-free water to a 50 μl volume, 250 μl lysis buffer, 1.75 μl 6-mercapto ethanol, 25 μl 2M sodium acetate (pH 4) and 175 μl ethanol (100%). The samples were then loaded into a RNA-binding column placed into a 2 ml collection tube and centrifuged at 16,000 g 30-60 sec. Following centrifugation, the column was discarded and 583 μl of 100% ethanol was added to the flow-through. Then, each sample was loaded into a new RNA-binding column placed into a 2 ml collection tube and centrifuged 30-60 sec at 16,000 g. Two loads of approximately 700 μl each were needed. Next, samples were washed first with 500 μl of low-salt wash buffer and centrifuged at 16.000 g for 30 sec and after discarding the flow-through with 300 μl of low-salt wash buffer were added and centrifuged at 16.000 g for 30 sec. After discarding the flow-through, 500 μl of fresh 80% ethanol was added in the column and centrifuged 30-60 sec followed by an additional 2 min centrifugation to dry the membrane. Last, 20 μl of RNAse-free water pre-heated at 60° C. directly onto the center of the column, incubated at room temperature for 2 min and centrifuged at 16.000 g for 1 min. Last, the column was reloaded with the flow-through onto the center of the column, incubated 2 min at room temperature and centrifuged at 16.000 g for 1 min. The flow-through containing the labeled small RNA fraction was kept on ice until used.

### Hybridization Procedures

Each of the small RNA enriched samples labeled with Cy3 was hybridized against a reference pool labeled with Cy5 containing total RNA from the above described tissue samples tumour samples, 61 tumor cell lines (59 tumor cell lines from the NCI-60 panel plus 2 other colorectal tumor cell lines) and placenta tissue. The rationale behind this common pool is to provide a reference signal to which all experiments can be compared. To make sure that a fold-change for every candidate miRNA can be calculated, the reference pool should give a positive signal for every candidate, which is ensured by including every sample in this pool.

The samples were prepared for hybridization by mixing equimolar amounts of Cy3 and Cy5-labelled samples Hybridization took place to a single subgrid of the 8×15K Agilent microarray under standard conditions. Prior hybridization, each sample was heated at 60° C. in the dark for 2 min, centrifuged at 13,000 rpm for 1 min and place on ice until it was loaded on the array. Hybridization took place at 37° C. for 17 hours at 10 rpm. Post hybridization washes were done at room temperature after careful removal of cover into the Wash solution 1 were the slides were kept for 1 min and then transfer to Wash solution 2, and wash for 1 min. Last, the slides were immersed into wash solution 3 for 30 sec and then dried by air. Images of the arrays were acquired by scanning at 5 micron resolution (Agilent DNA Microarray scanner G2505B-Agilent technologies, Palo Alto, USA).

### Array Design

Design of the microarray was based on 474 human pre-miRNA sequences From miRBase 9.1 and 3315 human pre-miRNA sequences from PCT/NL2006/0000010. Positions of mature miRNA sequences within pre-miRNA were known from previous public and private cloning data. In cases where only the mature sequence corresponding to one of the arms of the pre-miRNA was known, the corresponding star sequence was predicted by folding the pre-miRNA using RNAfold (Vienna RNA package, Hofacker Ill. (2003) Vienna RNA secondary structure server. Nucleic Acids Res. 31(13):3429-31) and selecting the sequence from the hairpin stem based on Dicer/Drosha cleaving properties (2 nt 3′ overhang) and the position of cloned mature miRNA sequence. The first 22 nt of every cloned and predicted mature miRNAs were used for designing probes complementary to the respective miRNAs. In cases where the mature miRNA was shorter than 22 nt, it was extended with the corresponding sequence from the pre-miRNA. Agilent custom microarrays (Wolber P K, Collins P J, Lucas A B, De Witte A, Shannon K W. (2006). The Agilent in situ-synthesized microarray platform. Methods Enzymol. 2006; 410:28-57) were designed with 60 nt probe sequences. Every probe carries spacers (CGATCTTT) and two copies of miRNA-specific sequence:

5′-22 nt-miR-CGATCTTT-22 nt-miR-CGATCTTT-3′
where 22 nt-miR is the reverse-complemented sequence of mature miRNAs selected as describe above. In addition, 13 non-overlapping 5S and 13 U6 22 nt sequences were included into array as controls. These sequences were manually selected from proprietary small RNA cloning data. In total the array contained (2×474)+(2×3315)+13+13=7604 probe sequences that were put in duplicates on every subgrid of the 8×15k Agilent custom microarrays. A complete list of all probes included in the microarray is given in Table 21

### Data Analysis

Array processing and spot-calling was performed automatically using Agilent's FeatureExtraction software (9.5.3). Background subtractions, array normalization and extraction of gene expression values were performed by limma package from Bioconductor (Tables 1, 8 and 13).

### Cluster Analyses (FIG. 1-3)

Supervised cluster analysis was done using TIGR multi experiment viewer (TMev), applying complete linkage and using Euclidian distances as metric

### Other Statistical Analyses

For comparing miRNA expression levels in colorectal adenomas and colorectal carcinomas, colorectal adenomas and controls, and colorectal carcinomas and controls a Wilcoxon signed-rank sum test was used with a threshold p-value of 0.2.

For comparing miRNA expression levels in colorectal tumors with and without defined chromosomal aberrations (i.e. 8p loss, 13q loss, 15q loss, 17p loss, 18q loss and 20q gain) a Wilcoxon signed-rank sum test was used with a threshold p-value of 0.3.

To correct for multiple comparisons a Benjamini-Hochberg correction was performed in all analyses.

Indicator miRNAs in Blood or Faeces (Tables 9-11 and 14-16)

Differentially expressed miRNAs (Tables 2-4) were compared to miRNAs expressed in faeces (Table 8) or blood (Table 13). Small RNAs that were expressed differentially in a significant manner (BH<0.2) in tissue and detectable in Faeces or blood (signal/background>2) were listed.

### Classification Analysis (Tables 5-7)

For multivariate classification of samples based on small RNA expression levels, PAM (prediction analysis for microarrays) software was used. PAM produces a classification rule using multiple variables (i.e. small RNAs) for optimal classification of predefined categories of samples (e.g. adenomas versus adenocarcinomas). PAM (Tibshirani et al. (2002) Proc Natl Acad Sci USA. 99:6567-72.) is a nearest centroid classification-type algorithm The training data is used to compute so-called class centroids. The centroid is a multivariate (since all expressed small RNAs are used) generalization of a median. Basically, a new sample would be classified to the class to which the squared distance is minimal.

Nearest shrunken centroid classification makes one important modification to standard nearest centroid classification. It “shrinks” each of the class centroids toward the overall centroid for all classes by an amount we call the threshold. This shrinkage consists of moving the centroid towards zero by a threshold, setting it equal to zero if it hits zero. After shrinking the centroids, the new sample is classified by the usual nearest centroid rule, but using the shrunken class centroids. This shrinkage does automatic selection of the most relevant small RNAs.

The error rates are computed by 10-fold cross-validation: 10 samples are left out of the training procedure and their class label is predicted as descibed above. The procedure is set up such that each sample is left out exactly once. The confusion table then displays the number of correct and erronous classifications for both classes.

Pairwise comparisons were performed (control vs adenoma, control vs carcinoma and adenoma vs carcinoma) followed by feature selection. Then, cross-validated misclassification errors were computed (see the confusion tables 5b-7b). This shows which miRNAs were vital for a high accuracy of prediction the type of tissue (control vs adenoma vs carcinoma) and the accuracy of the prediction (%). Subsequently, the selected feature miRNAs were eliminated from the data and the classification was redone. The cross-validated misclassification errors were computed again. An increase in error shows that the accuracy of the classification relies on the presence of the featured miRNAs.

### Results

### Small RNA Fraction Detectable in Colorectal Tissue

A large set of novel small RNAs have recently been experimentally identified at the Hubrecht Institute, Utrecht, The Netherlands, and classified as candidate miRNAs using computational analysis (e.g. stable hairpin formation of the precursor) (Berezikov et al. (2005) Cell 120:21-24, Berezikov et al. (2006) Nature Gen. 38: 1375-1377, Berezikov et al. (2006) Genome Res. 16: 1289-1298, PCT/NL2006/0000010). Earlier studies have shown that expression profiling of small RNAs can be used to classify tumors and use it as a diagnostic tool. In colorectal cancer, early diagnosis could make an enormous difference in life expectancy, morbidity and mortality. To determine whether these novel small RNAs, which are suspected to be miRNAs, are differentially expressed in different types of cancer, analysis of the expression of these small RNAs was performed in different colorectal tissues. The signals detected for the miRNAs and small RNAs obtained for the tissue samples were compared to signals obtained using the reference sample. Table 1 shows the result of this comparison. In total, 1296 probes produced signal with difference between maximum and minimum values above 4 in log 2 scale, indicating substantial differential expression of corresponding small RNAs in at least some of the investigated colorectal tissues.

### Differentially Expressed Small RNAs in Diseased Colorectal Tissue Versus Healthy Colorectal Tissue

In order to determine which small RNAs might serve as disease markers and may consequently be used for diagnostic purposes, detection signals of the analyzed small RNAs were compared between colorectal adenomas and control samples, colorectal adenocarcinomas and control samples and colorectal adenomas and adenocarcinomas. This comparison resulted in a list of small RNAs that are differentially expressed between the respective categories analysed (Tables 2-4). Moreover, Tables 2b-4b demonstrate which of the differentially expressed small RNAs are upregulated and which are downregulated. In summary, a comparison of colorectal adenomas and control samples showed that 1554 small RNAs are differentially expressed, of which 50 are upregulated (Table 2c) and 1504 are downregulated (Table 2d) in adenomas. The comparison of colorectal adenocarcinomas and control samples demonstrated that 105 small RNAs are differentially expressed (Table 3a), of which 23 are upregulated (Table 3c) and 82 are downregulated (Table 3d) in adenocarcinomas. Lastly, comparing colorectal adenomas and adenocarcinomas resulted in a list of 1080 small RNAs that are differentially expressed (Table 4a), of which 1 is upregulated (Table 4c) and 1079 are downregulated (Table 4d) in adenocarcinomas. When the statistical analysis was performed under more stringent criteria (p<0.1), a list of 384 small RNAs was selected, that are most significantly differentially expressed between adenoma and adenocarcinoma (Table 26). All of these small RNAs were downregulated in adenocarcinomas. The significance of these results is reiterated by the fact that, when cluster analysis (Euclidean) is performed, using all differentially expressed small RNAs, two distinct clusters are formed (Figure I-III). Figure Ia shows cluster analysis based on all differentially expressed small RNAs between control and adenomas. Cluster analysis shows two distinct clusters: one which included mostly adenomas (Cluster II) and one including mostly controls (Cluster I). This demonstrates that on the basis of the differentially expressed small RNAs it is possible to distinguish these two different tissue types. Statistical analysis (FIG. 1b) demonstrates that the different tissue samples are assigned to the correct cluster in a significant manner. Importantly, the cluster analysis was repeated with a lower number of differentially expressed small RNAs (p<0.1, p<0.05 and p<0.025 or best 23 predictive, differentially expressed small RNAs) as a result of more stringent statistical criteria (p value linked to the differentially expressed small RNAs is decreased). The results of those analyses show that it remains possible to distinguish, in a significant manner, two clusters containing mainly controls in one and mainly adenomas in the other (FIG. 1c-h), although the clusters do change somewhat when the number of included small RNAs decreases. Figures II and III, clustering adenocarcinoma and control samples and adenoma and adenocarcinoma samples respectively, show similar results as described for Figure I. These data imply that the selected small RNAs that are differentially expressed have great value for diagnostic purposes and will be further developed as such.

In conclusion, these results show differences in small RNA content between the respective categories of samples (normal colorectal epithelium, adenomas and adenocarcinomas) tested. They further show that the selected small RNAs that are differentially expressed have great value for clinical diagnostic purposes and will be further developed as such. Based on the expression of the different small RNAs identified above, it becomes possible to classify an unknown tissue sample into a normal category an adenoma category or an adenocarcinoma category depending on its small RNA type expression profile. Such a comparison may then result in an accurate diagnosis of normal vs adenoma vs adenocarcinoma. In addition, individual or groups of small RNAs may actually drive (the set of upregulated small RNAs, table 2c, table 3c and table 4c) or inhibit (downregulated small RNAs, table 2d, table 3d, table 4d) the transition from adenoma to carcinoma and may therefore function as targets for therapy.

### Classification of Diseased Colorectal Tissue

The subpopulations of small RNAs that are differentially expressed as identified above are still relatively large. This might hamper its use as a diagnostic tool in practice. Therefore, we determined which of the differentially expressed small RNAs are actually most predictive in its classification of a tissue sample in the categories normal, adenoma or adenocarcinoma. The selection of the most predictive small RNAs for multivariate classification of samples based on small RNA expression levels, PAM (prediction analysis for microarrays) software was used. PAM produces a classification rule using multiple variables (i.e. small RNAs) for optimal classification of predefined categories of samples (e.g. adenomas versus adenocarcinomas). It is well-known that with these high dimensional data one may obtain competitive classifiers with (almost) no overlapping features (small RNAs in this case). To assess the importance of the selected small RNAs as a set, we deleted these from the data set and repeated the classification procedure. We observe that the error rates drop substantially, which indicates that the initial set of small RNA does contain features which are essential for discriminating the two groups.

The results of these analyses are shown in Tables 5-7. These tables show lists of small RNAs that have a high predictive value. They are ranked, with the first being the small RNA with the highest predictive value. This predictive value is represented by the largest difference between the adenoma score and the adenocarcinoma score as listed in Tables 5a-7a. The predictive value and power of the complete set of small RNAs selected is due to the combination of these particular small RNAs. Table 5a shows that 55 from the selection of 1554 differentially expressed small RNAs in colorectal adenoma vs control are highly predictive and classify the tissue samples accurately in 80% of the cases (overall error rate 0.212, table 5b). This is exemplified by the fact that the accuracy rate decreases to 65% (overall error rate 0.349) when these 55 small RNAs are not included in the small RNA population used to classify tissue, demonstrating that these 55 contain good classifiers, to accurately predict the type of tissue analyzed. Table 6a shows that 79 from the selection of 105 differentially expressed small RNAs in adenocarcinoma vs control samples are highly predictive and classify the tissue samples accurately in 80% of the cases (overall error rate 0.198, Table 6b). This is exemplified by the fact that the accuracy rate decreases to 65% (overall error rate 0.365) when these 79 small RNAs are not included in the small RNA population used to classify tissue. Importantly, when these good predictors are excluded, it results in wrongly classifying 8 controls as an adenocarcinoma compared to 2 wrongly classified controls when the good predictors are included: the false positive rate increases significantly from 0.222 to 0.888. These data show that these 79 probably contain good classifiers, to accurately predict the type of tissue analyzed. Table 7a shows that 26 from the selection of 105 differentially expressed small RNAs in colorectal adenomas vs adenocarcinomas are highly predictive and classify the tissue samples accurately in 80% of the cases (overall error rate 0.198, table 7b). This is exemplified by the fact that the accuracy rate decreases to 65% (overall error rate 0.365) when these 26 small RNAs are not included in the small RNA population used to classify tissue. Moreover, exclusion of these small RNAs results in the increase of the false negative rate of 0.258 to 0.483 as indicated by double the adenocarcinomas that are wrongly classified as adenomas when excluding the 26 small RNAs. These results show that these 26 contain good classifiers to accurately predict the type of tissue analyzed. In conclusion, the described small RNAs (Tables 5a-7a) are minimal sets of small RNAs part or all of which are included in a diagnostic test, which could classify an unknown sample into the different categories (normal, colorectal adenoma, adenocarcinoma). The above described PAM analysis resulted in a classification rule that may be used in a diagnostic application. Such an application makes use of the small RNA content and expression profiles of an unknown sample which will be compared to the above identified combination of small RNAs using advanced algorithms. This calculates whether the final value is below or above a certain threshold that determines the consequent diagnosis. The threshold depends on the required specificity and sensitivity of the final diagnostic test.

### Detection of Small RNAs in Faeces and Blood

To design a functional and practical diagnostic test it is important to take into account the type of sample that is to be analyzed. Existing commercial tests are based on the analysis of stool samples. They have yielded promising results but need further improvement. Fecal occult blood test is the most recognized method of selecting high risk individuals. Unfortunately, with FOBT a considerable percentage of the tumours is missed and the already bleeding tumour is often discovered at a relatively late stage. A diagnostic test based on the detection of small RNA can be performed using tissue samples (biopsies) as described above. However, it is preferred to use test samples that can be obtained by less invasive measures. Stool samples and blood samples obtained from suspected colorectal patients could qualify. We therefore tested whether small RNAs derived from the tumor tissue are traceable in either blood or faeces. Therefore, we first determined if it was possible to extract small RNAs from blood and faces. The results are shown in tables 8 and 13. It shows the ratio of detection signal of the different small RNAs over background. For this calculation, processed background and signal values produced by FeatureExtraction software (Agilent) were used. We considered all signals with a ratio of 2 over background a significant signal. It demonstrates that 3759 small RNAs can be detected in stool sample and 3644 small RNAs in whole blood obtained from a patient with a colorectal adenocarcinoma. Interestingly, of some hairpin RNAs both arms of the hairpin could be detected, where one arm is higher expressed than the other, clearly resembling endogenous miRNA processing, where the mature strand gives stronger signals than the star sequence (see tables 8b and 13b). Secondly, we analyzed which small RNA, expressed in faeces and blood, are differentially expressed in comparing the three categories (colorectal adenomas and control samples, colorectal adenocarcinomas and control samples and colorectal adenomas and adenocarcinomas). These analyses should indicate which small RNAs are indicative of abnormality and can be detected in faeces or blood. They are based on the combination of Tables 8 and 13, including all small RNAs expressed twice above background, and Tables 2-4, including all differentially expressed small RNAs (p<0.2). A summary of Tables 9-11 for faeces and 14-16 for blood is given below (Table 17). The table shows that it is possible to recover small RNAs in blood and faeces that are also differentially expressed in tissue. In conclusion, both blood and faeces obtained from suspected colorectal patients are suitable samples for use in diagnostic small RNA based microarray tests. This is once again exemplified by the fact that part of the group of small RNAs identified as good classifiers can also be detected in faeces (Table 12) and blood (Table 18). Importantly, a comparison of these subsets presumable derived from colorectal cancer cells and detected in blood and faeces, learns that blood and faeces have 85-90% of these predictive small RNAs in common. This shows that these small RNAs are indeed derived from colorectal adenocarcinoma cells and more importantly, that both blood and faeces are suitable for detecting colorectal carcinoma derived small RNAs.

Correlation of Expression of Small RNAs with Chromosomal Aberrations

The colorectal tissue samples used in this study have further been characterized on other molecular levels. By means of array CGH analysis it had been determined which of the above studied adenomas and adenocarcinomas are characterized by chromosome instability, and which aberrations specifically. It was shown that 8q, 13q and 20q gains and 8p, 15q, 17p and 18q losses, are associated with progression of colorectal adenomas to carcinomas (Hermsen et al. (2002) cited above). Therefore, we determined whether the chromosomal aberrations frequently reported in colorectal cancer are correlated with the expression of small RNAs. Table 19 shows that two typical chromosomal aberrations, loss of X8p and loss of X17p, correspond to significant expression of 219 and 15 small RNAs respectively. One could view the significance of this correlation of small RNA expression with certain chromosomal aberration in two manners; the first would regard small RNAs correlated with certain chromosomal aberrations as indicators for those aberrations and could therefore be used in diagnostic tests based on such chromosomal instabilities. The second way of regarding this correlation is to take into account the biological consequences. The loss of X8p and X17p affected expression of these particular small RNAs. This could either be a direct consequence: these small RNAs are encoded by the part of these chromosomes that is lost. It could also be an indirect effect: the chromosomes that are lost encode for genes that regulate the expression of these small RNAs. Moreover, the fact that loss of these chromosomes affects clinical behaviour of tumors may suggest that the small RNAs, correlated with this loss, have a biological function in the process of tumorigenesis.

Currently, the small RNA expression profiles identified in these 68 colorectal tissue samples have been analyzed to identify good clinical classifier and correlated with small RNAs recovered from faeces and blood obtained from colorectal adenocarcinoma patients, to determine if a diagnostic small RNA based test would be feasible. Moreover, these expression profiles were combined with array CGH data, determining whether chromosomal aberrations can be correlated with certain small RNAs. It is hypothesized that the small RNAs correlated with loss of X8p and X17p (as identified in table 19) will also have a biological function, since loss of these chromosomal aberrations is correlated with clinical prognosis. Therefore, in the future, these small RNAs will be studied in more detail to test that hypothesis. Importantly, the correlation between small RNA expression profiles and already available mRNA expression profiles will be determined shortly. This may give indications on which genes are targeted by the small RNAs that are identified in colorectal adenomas and adenocarcinomas. It is of particular interest to determine the target genes of small RNAs that are differentially expressed between adenomas and adenocarcinomas. These small RNAs may be involved in progression to adenocarcinomas and may form interesting targets for drug discovery. Consequently, the small RNA gene targets may therefore also be of great interest.

### Example 2

### Materials and Methods

For materials and methods for the PAM analysis, represented in table 25, we refer to the materials and methods as presented under example 1.

### Results

To confirm the classifier as listed in table 7a, the PAM analysis was performed again, this time using a double cross validation to assure accurate prediction of the most predictive small RNAs. The small RNAs as listed in table 25a are the result of this analysis. 41 small RNAs were selected from the list of differentially expressed small RNAs. The combination of these 41 small RNAs is able to distinguish adenomas from adenocarcinomas in a highly predictive manner with a 72% accuracy (overall error rate 0.283, Table 25b). This is exemplified by the fact that the accuracy rate decreases to 66% (overall error rate 0.34) when these 41 small RNAs are not included in the small RNA population used to classify tissue, demonstrating that these 41 contain good classifiers, to accurately predict the type of tissue analyzed. The above described PAM analysis resulted in a classification rule that may be used in a diagnostic application. Such an application makes use of the small RNA content and expression profiles of an unknown sample which will be compared to the above identified combination of small RNAs using advanced algorithms. This calculates whether the final value is below or above a certain threshold that determines the consequent diagnosis. The threshold depends on the required specificity and sensitivity of the final diagnostic test.

To improve the predictive value of such classifiers as described above, a number of strategies can be followed. The first strategy could be to increase the sample set to ensure the correct selection of predictive small RNAs. This strategy could in some cases increase the accuracy of prediction and decrease the error rate. However, in other cases the predictive value of a diagnostic test has already reached its limit and increasing the sample set will not result in a more accurate predictive set. Therefore, a second strategy to improve the predictive value of the diagnostic test may be to combine the dataset based on the microarray data with other data sets. For example, a diagnostic test may combine data based on the small RNA expression levels with array CGH data obtained from the same samples. This could result in a more accurate prediction of disease state. A third option may be to use combined diagnostic tests in a step-wise procedure, where the first diagnostic test will have the best predictive value. For patients that fall within a category for which no clarity as to diagnosis can be given, a second diagnostic test will follow that will ultimately result in a diagnosis of the disease state. The second test on its own would not have given a clear answer, but in combination with the first test, this resulted in a defined and reliable diagnosis.

Importantly, of these 41 classifier small RNAs, a portion of them can be detected in faeces (Table 34) or blood (Table 35). As mentioned, both blood and faeces obtained from suspected colorectal patients are suitable samples for use in diagnostic small RNA based microarray tests. The presence of small RNAs identified as good classifiers in faeces and stool suggests that the classifier can be used as a diagnostic test using faeces or blood as a source for small RNAs derived from colorectal adenomas or adenocarcinomas.

### Example 3

### Materials and Methods

For materials and methods for the cluster analysis, represented in Fig IV, we refer to the materials and methods as presented under example 1.

### Results

From the 384 small RNAs that were found to be most significantly differentially expressed between adenomas and adenocarcinomas (Fig IIIc, d and table 26), 91 were selected and believed to be highly predictive for classification of adenoma vs adenocarcinoma (Table 27). This was reiterated by the fact that, when cluster analysis (Euclidian) was performed using the 91 small RNAs, two distinct clusters are formed (Fig IVa). Moreover, statistical tests showed (Chi-square test) that the clustering based on the 91 selected small RNAs is highly significant (P<0.0001) (Fig IVb).

### Example 4

### Materials and Methods

### Patient Samples

Fresh colorectal tissue samples from 58 patients with colorectal tumors were collected prospectively at the department of pathology of the VU-University medical center (VUmc), Amsterdam, the Netherlands. In total 27 adenomas and 31 adenocarcinomas, of which 12 adenomas and 12 carcinomas were also analyzed by small RNA based microarray (see example 1). Of all samples, total RNA was isolated using TRIzol (Invitrogen) following the manufacturer's guidelines with some modifications (http://www.vumc/nl/microarrays/index.html). Total RNA quantity was determined with a Nanodrop ND-1000 spectrophotometer (Isogen, Hackensack, N.J., USN and quality was assessed in a 1% agarose gel, stained with ethidium bromide. The study was carried out in accordance with the ethical guidelines of our institution concerning the use of patient material.

TaqMan PCR

Quantification of the left arm of hsa-miR-24-2 (hsa-miR-24-2-1) expression levels was done by real-time RT-PCT using Tagman® microRNA assays (Applied Biosystems, Foster City, Calif., USA) directed to hsa-mir-24 (ABI4 373072) and an endogenous reference, the RNU48 gene (ABI 437338). Reactions were performed following the manufacture's protocol using 10 ng of total RNA as input material. All reactions were carried out in duplo in a 7300 Real-time PCR System (Applied Biosystems, Foster City, Calif., USA).

### Statistical Analysis

Expression levels of the hsa-miR-24-2-1 were calculated from the obtained Ct values using the delta Ct method as previously described. Box and scatter plots were used to appreciate the descriptive statistics of the data (SPSS 14.0 for Windows, SPSS Inc. Chicago, Ill., USA). Significance of differences in expression levels between adenomas and carcinomas were computed by the Mann-Whitney U non-parametric test for independent samples (SPSS 14.0 for Windows, SPSS Inc. Chicago, Ill., USA).

### Results

To validate the microarray expression data as presented in FIGS. 1-3, the expression levels of mir-24-2-1, for which primers were commercially available, were determined by real-time RT-PCR in 58 colorectal tumors (27 adenomas and 31 adenocarcinomas). Expression levels of hsa-miR-24-2-1 showed 1.4 fold increased expression in the adenomas (mean expression 13.8) compared to the carcinomas (mean expression 12.2) (p=0.4) (FIG. 5A). This observation was in concordance with the results from the microarray expression experiment, where the expression levels of the hsa-mir-24-2-1 were higher in group of adenomas (mean expression 1.4) compared to carcinomas (mean expression 1.0) (p=0.01) (FIG. 5B).

### Example 5

Colorectal cancer (CRC) results from the accumulation of DNA copy number gains and losses, promoter methylation changes, mutations and alterations in microRNAs (miRNAs) expression. It is well documented that 85% of the colorectal tumours show genetic instability through specific chromosomal gains and losses of small or large portions or whole chromosomes. These tumours are known as chromosomal instable (CIN) tumours. The biological consequences of the described DNA copy number changes that characterize CRC have not been fully established since the genes or miRNAs with tumour suppressor or oncogenic function, which are located in those regions have not been fully identified. In the last years, small RNA molecules have shown to play an important role in the pathogenesis of human cancers as oncogenes and tumours suppressor genes. Small RNAs contribute to the pathogenesis of human cancers due to their altered expression. The cause of their altered expression has only been partially characterized. It is known that DNA copy number changes, epigenetic changes and transcription factors involved in human carcinogenesis lead to increase or decrease expression of small RNAs during cancer initiation and progression. Therefore, small RNAs located in loss and gain regions characteristic of CRC as well as small RNAs which expression is associated to DNA copy number changes, may play a role as causative oncogenes or tumour suppressor genes in CRC. Identification of the small RNAs that participate as causative oncogenes and tumour suppressor genes in DNA copy number gain and loss regions will lead to a better understanding of the molecular mechanisms underlying CRC pathogenesis and may both serve as highly specific biomarkers for CRC or as possible targets for pharmaceutical intervention with the development of CRC.

### Materials and Methods

### Materials

38 colorectal carcinomas with array CGH data and microsatellite instability status available were collected at the Zaans Hospital, Zaandam, the Netherlands and Leeds University, UK. The study was carried out in accordance with the ethical guidelines of our institution concerning the use of patient material.

### DNA Isolation

DNA from healthy mucosa and tumor DNA of each patient was isolated using the QIAmp microkit (Qiagen, Westburg, Leusden, the Netherlands) as previously described. (Weiss M M, Hermsen M A, Meijer G A, et al. Comparative genomic hybridisation. Mol Pathol 1999; 52:243-51. Buffart T E, Carvalho B, Hopmans E, et al. Gastric cancers in young and elderly patients show different genomic profiles. J Pathol 2007; 211:45-51).

### Microsatellite Instability Analysis

Microsatellite Instability analysis was determined by using the MSI Analysis System, MSI Multiplex System Version 1.2 (Promega, Madison, Wis., USA) containing five monomorphic markers (BAT-25, BAT-26, NR-21, NR-24, MONO-27). Reactions were performed following the manufacturer's instructions. The obtained PCR products were separated on a ABI 3130 DNA sequencer (Applied Biosystems, Foster City, Calif., USA) and analyzed by GeneScan 3100 (Applied Biosystems, Foster City, Calif., USA). An internal lane size standard was added to the PCR samples for accurate sizing of alleles and to adjust for run-to run variations. Tumours were considered as microsatellite instable (MSI) when two or more markers ((MSI-H) showed length changes (instability). Tumours showing none or only one instable marker (MSI-L) were considered as Microsatellite stable (MSS).

### Array Comparative Genomic Hybridization

Array CGH was performed on 30K oligonucleotide arrays as described before (Snijders A M, Nowak N, Segraves R, et al. Assembly of microarrays for genome-wide measurement of DNA copy number. Nat Genet. 2001; 29:263-4. Van den Ijssel P, Tijssen M, Chin S F, et al. Human and mouse oligonucleotide-based array CGH. Nucleic Acids Res 2005; 33:e192). Briefly, 600 ng of tumour and normal DNA were differentially labelled by random priming (Bioprime DNA Labeling System, Invitrogen, Breda, the Netherlands). Unincorporated nucleotides were removed with Sephadex columns (ProbeQuant G-50 Micro Columns, Amersham Biosciences) and 50 μl of tumour and normal DNA were combined with 10 μg Cot-1 DNA and precipitated by adding 2.5 volumes of ice-cold 100% ethanol and 0.1 volume of 3M sodium acetate (pH 5.2). The DNA was collected by centrifugation at 14000 rpm for 30 minutes at 4° C. The pellet was dissolved in 130 μl hybridization solution and incubated at 73° C. for 10 minutes to denature the DNA, followed by 60-120 minute incubation at 37° C. Next, the hybridization mixture was added to the array in a hybridization station (HybArray12™, Perkin Elmer Life Sciences, Zaventum, Belgium) and incubated for 38 hours at 37° C. After hybridization the slides were washed in six washing steps and dried by centrifugation for 3 minutes at 1000 g.

### Image Acquisition and Data Analysis

Images of the arrays were acquired by scanning (Agilent DNA Microarray scanner, Agilent Technologies, Palo Alto, USA) and Bluefuse software version 3.4 (BlueGnome, Cambridge, UK) was used for automatic feature extraction. Spots were excluded when the quality flag was below 1 or the confidence value was below 0.1. Log 2 tumour to normal ratio was calculated for each spot and normalized against the mode of the ratios of all autosomes. For determining copy number gains and losses, the R package CGH call was used (van de Wiel M A, Kim K I, Vosse S J, et al. CGHcall: calling aberrations for array CGH tumor profiles. Bioinformatics 2007; 23:892-4).

### Statistical Analysis

ACE-it (Array CGH Expression integration tool) was applied to statistically test whether gene dosage affects microRNA expression (van Wieringen et al., 2006). This tool was applied to the whole genome data. We used a cut-off value 0.15 for gains and losses, a default group value of 9 and an FDR for significance of 0.10.

### Results

### Localization of Small RNAs in Microdeletion or Microgain Regions in Colorectal Carcinoma

Eight microdeleted regions, smaller than 3 megabases, were observed in at least two of the 38 colorectal carcinomas. To determine whether the changes in small RNA expression in colorectal carcinomas may be a consequence of their location in the previously mentioned microdeletions or microgains (smaller than 3 Mb), the localization of small RNAs in such regions detected by array CGH was ascertained. A significant amount of small RNAs appear to be located in these eight deleted regions (table 28a). Out of this list of small RNAs, 6 small RNAs were shown to be differentially expressed between adenomas and carcinomas (table 28b). Due to the location of the small RNA in these specific regions, these small RNAs could therefore contribute causally to colorectal carcinogenesis as tumor suppressor genes or oncogenes and form targets for therapeutic intervention or be used as highly specific biomarkers. Reintroduction of such, previously deleted, small RNAs may benefit the patient by affecting tumor progression or response to therapy and therefore clinical outcome. Moreover, they could be used as biomarkers for early diagnosis, therapy response or clinical outcome of colorectal carcinomas. Five micro-gain regions, smaller than 3 megabases, were observed in at least two of the 38 colorectal carcinomas. In these 5 regions, 22 small RNAs are located (Table 29a), one of which has been shown to be differentially expressed between adenomas and carcinomas (Table 29b). Gain of these small RNAs may be causatively involved in the initiation and maintenance of colorectal tumours. As such, inhibition of these small RNAs could result in the opposite effect and provide a novel therapeutic strategy for the treatment of colorectal carcinomas. In addition, these microRNAs could be used as biomarkers for molecular diagnosis or tumor classification regarding therapy response, therapeutical intervention or clinical outcome.

### Localization of Small RNAs in Microdeletion or Microgain Regions in Colorectal Microsatellite Stable Carcinomas

Within the population of colorectal carcinomas, two groups can be distinguished. One group that has stable microsatellites, the other group is defined by instable microsatellites. The microsatellite instability is often a result of DNA mismatch repair deficiencies and therefore, the status of stability of the microsatellites determines the sensitivity of these tumors for the accumulation of mutations or epigenetic changes. To determine whether microdeletions or gains can be found specifically in one group or the other, array CGH data was used to determine such changes in these two groups.

In at least two out of 26 microsatellite stable carcinomas, two micro-deletions, smaller than 3 megabases, were observed. Four small RNAs are located in these regions (table 30). Three micro-gain regions, smaller than 3 megabases, were observed in at least two of the 26 microsatellite stable colorectal carcinomas. In these regions, 10 small RNAs are localized (table 31a), of which 1 is differentially expressed in adenomas compared to carcinomas (table 31b). It is known that MSI and MSS have different clinical outcome and respond differently to the current chemotherapy regimens. On this basis, small RNAs located in microdeleted or microgained chromosomal regions that are most frequently present in MSS tumours, might be responsible for the different clinical behaviour seen in the MSS colorectal tumors. Therefore, small RNAs that are located in these gain and loss regions are highly suitable for use as biomarkers to distinguish microsatellite stable from microsatellite instable carcinomas, for early diagnosis of these tumors or tumor classification regarding therapy response, to dispense the most suitable therapeutical regimen for the patients or predict clinical outcome.

### Localization of Small RNAs in Microdeletion or Microgain Regions in Colorectal Microsatellite Instable Carcinoma

Eight micro-deletions, smaller than 3 megabases, were observed in at least two of the 12 microsatellite instable colorectal carcinomas. In these deleted regions, 41 small RNAs are localized (table 32a), of which 4 are differentially expressed between adenomas and carcinomas (table 32b). No microgains could be detected in this group of colorectal carcinomas. These 41 small RNAs that are localized in deleted regions, and in particular the 4 that are differentially expressed, may contribute to the maintenance of the tumor phenotype. Therefore, they could be suitable for therapeutic approaches, where reintroduction of these small RNAs may intervene with tumor growth or other tumor specific characteristics. Moreover, these small RNAs may serve as highly specific biomarkers since it is known that MSI and MSS have different clinical outcome and respond differently to the current chuemotherapy regimens. On this basis, small RNAs located in microdeleted chromosomal regions that are most frequently present in MSI tumours, might be responsible for the different clinical behaviour seen in the MSI colorectal tumors. Therefore, small RNAs that are located in these microdeleted regions are highly suitable for use as biomarkers to distinguish microsatellite stable from microsatellite instable carcinomas, for early diagnosis of these tumors or tumor classification regarding therapy response, to dispense the most suitable therapeutical regimen for the patients or predict clinical outcome.

### Integration of Small RNAs Expression and Array Comparative Hybridization Data.

BAC array CGH data were related to small RNA expression array data as presented in Example 1, independently of adenoma or carcinoma status. Therefore, a dedicated integration tool called ACE-it was applied (van Wieringen et al., 2006). We obtained a list of five small RNAs for which DNA dosage affected expression levels (FDR<0.1) (Table 33). The expression of these five small RNAs is associated to DNA copy number changes, located on chromosomal regions implicated in colorectal pathogenesis (Beatriz Carvalho, Cindy Postma, Sandra Mongera, Erik Hopmans, Sharon Diskin, Mark A. van de Wiel, Wim van Criekinge, Olivier Thas, Anja Matthai, Miguel A. Cuesta, Jochim S. Terhaar sive Droste, Mikael Edward Craanen, Evelin Schrock, Bauke Ylstra, and Gerrit A. Meijer. Multiple putative oncogenes at the chromosome 20q amplicon contribute to colorectal adenoma to carcinoma progression (in press)). This indicates that these 5 small RNAs may be involved and causal in colorectal carcinogenesis. Small RNAs which expression is associated to DNA copy number changes may contribute causally to colorectal carcinogenesis as tumor suppressor genes or oncogenes and form targets for therapeutic intervention or be used as highly specific biomarkers. Moreover, they could be used as therapeutical targets or be used as biomarkers for early diagnosis, therapy response or clinical outcome of colorectal carcinomas.

### Brief Description of the Tables

### Brief Description of the Tables

### Tables

1. Differentially expressed small RNAs in colorectal samples vs reference pool.
   For every probe, samples with minimal and maximal values of log 2(sample/reference) were selected and amplitude of differential expression was calculated by subtracting minimal value from maximal. Probes with the amplitude above 4 were considered as differentially expressed.
2. Differentially expressed small RNAs in colorectal adenoma vs control
   A. Small RNAs that are differentially expressed between control (brush sample of resected colorectal margins) and adenoma. A wilcoxon analysis was performed adjusting the p values with a Benjamini-Hochberg correction. Small RNAs are differentially expressed when the adjusted p value is below 0.2
   B. Up- or downregulated small RNAs adenoma compared to control
   C. List of small RNAs upregulated in adenoma compared to control. A small RNA is considered upregulated when the ratio is above 1
   D. List of small RNAs downregulated in adenoma compared to control. A small RNA is considered downregulated when the ratio is below 1.
3. Differentially expressed small RNAs in colorectal adenocarcinoma vs control
   A. Small RNAs that are differentially expressed between control (brush sample of resected colorectal margins) and adenocarcinoma. A wilcoxon analysis was performed adjusting the p values with a Benjamini-Hochberg correction. Small RNAs are differentially expressed when the adjusted p value is below 0.2
   B. Up- or downregulated small RNAs adenocarcinoma compared to control
   C. List of small RNAs upregulated in adenocarcinoma compared to control. A small RNA is considered upregulated when the ratio is above 1
   D. List of small RNAs downregulated in adenocarcinoma compared to control. A small RNA is considered downregulated when the ratio is below 1
4. Differentially expressed small RNAs in adenoma vs adenocarcinoma
   A. Small RNAs that are differentially expressed between adenoma and adenocarcinoma. A wilcoxon analysis was performed adjusting the p values with a Benjamini-Hochberg correction. Small RNAs are differentially expressed when the adjusted p value is below 0.2
   B. Up- or downregulated small RNAs adenocarcinoma compared to adenoma
   C. List of small RNAs upregulated in adenocarcinoma compared to adenoma. A small RNA is considered upregulated when the ratio is above 1
   D. List of small RNAs downregulated in adenocarcinoma compared to adenoma. A small RNA is considered downregulated when the ratio is below 1
5. Classification on the basis of predictive small RNA subset for distinction between normal tissue and adenoma
   A. List of small RNAs that are selected by PAM as good classifiers to distinguish control from adenoma tissue
   B. Confusion tables before and after exclusion of the selected set of small RNAs
6. Classification on the basis of predictive small RNA subset for distinction between normal tissue and adenocarcinoma
   A. List of small RNAs that are selected by PAM as good classifiers to distinguish control from adenocarcinoma tissue
   B. Confusion tables before and after exclusion of the selected set of small RNAs
7. Classification on the basis of predictive small RNA subset for distinction between adenoma and adenocarcinoma
   A. List of small RNAs that are selected by PAM as good classifiers to distinguish adenoma from adenocarcinoma tissue
   B. Confusion tables before and after exclusion of the selected set of small RNAs
8. Recovered small RNAs in faeces. For every probe, ratio of processed signal above background, as determined by FeatureExtraction software, was calculated, and probes with the ratio above 2 were considered as positively detected in the sample.
9. Small RNAs recovered from faeces and differentially expressed in adenoma vs control tissue
   This table includes small RNAs that were expressed differentially in a significant manner (p value<0.2) in adenoma compared to control and are detectable in faeces (signal/background>2) were listed.
      A. Small RNA hairpin arm that shows highest expression with signal/background>2
      B. Small RNA hairpin arm that shows lowest expression with signal/background>2
10. Small RNAs recovered from faeces and differentially expressed in adenocarcinoma vs normal tissue
   This table includes small RNAs that were expressed differentially in a significant manner (p value<0.2) in adenocarcinoma compared to control and are detectable in faeces (signal/background>2) were listed.
      A. Small RNA hairpin arm that shows highest expression with signal/background>2
      B. Small RNA hairpin arm that shows lowest expression with signal/background>2
11. Small RNAs recovered from faeces and differentially expressed in adenocarcinoma vs adenoma
   This table includes small RNAs that were expressed differentially in a significant manner (p value<0.2) in adenocarcinoma compared to adenoma and are detectable in faeces (signal/background>2) were listed.
      A. Small RNA hairpin arm that shows highest expression with signal/background>2
      B. Small RNA hairpin arm that shows lowest expression with signal/background>2
12. Presence of classifiers in faeces
   This table includes small RNAs that were selected as good classifiers in tables 5-7 and could be detected in faeces
      A. Classifiers for adenoma vs control detectable in faeces
      B. Classifiers for adenocarcioma vs control detectable in faeces
      C. Classifiers for adenoma vs adenocarcinoma detectable in faeces
13. Recovered small RNAs in blood.
   For every probe, ratio of processed signal above background, as determined by FeatureExtraction software, was calculated, and probes with the ratio above 2 were considered as positively detected in the sample.
14. Small RNAs recovered from blood and differentially expressed in adenoma vs normal tissue
   This table includes small RNAs that were expressed differentially in a significant manner (p value<0.2) in adenoma compared to control and are detectable in blood (signal/background>2) were listed.
      A. Small RNA hairpin arm that shows highest expression with signal/background>2
      B. Small RNA hairpin arm that shows lowest expression with signal/background>2
15. Small RNAs recovered from blood and differentially expressed in adenocarcinoma vs normal tissue
   This table includes small RNAs that were expressed differentially in a significant manner (p value<0.2) in adenocarcinoma compared to control and are detectable in blood (signal/background>2) were listed.
      A. Small RNA hairpin arm that shows highest expression with signal/background>2
      B. Small RNA hairpin arm that shows lowest expression with signal/background>2
16. Small RNAs recovered from blood and differentially expressed in adenocarcinoma vs adenoma
   This table includes small RNAs that were expressed differentially in a significant manner (p value<0.2) in adenocarcinoma compared to adenoma and are detectable in blood (signal/background>2) were listed.
      A. Small RNA hairpin arm that shows highest expression with signal/background>2
      B. Small RNA hairpin arm that shows lowest expression with signal/background>2
17. Summary of results shown in Tables 8-16
18. Presence of classifiers in blood
   This table includes small RNAs that were selected as good classifiers in tables 5-7 and could be detected in blood
      A. Classifiers for adenoma vs control detectable in blood
      B. Classifiers for adenocarcinoma vs control detectable in blood
      C. Classifiers for adenoma vs adenocarcinoma detectable in blood
19. Correlation of expressed small RNAs with chromosomal aberrations in adenoma and adenocarcinoma samples
   A. Small RNAs correlated with a loss of chromosome 8p. No correlation could be detected between these small RNAs and loss of chromosome 17p or gain of 13q (see table) or gain of 15q, 18q or 20q (data not shown).
   B. Small RNAs correlated with a loss of chromosome 17p. No correlation could be detected between these small RNAs and loss of chromosome 8p or gain of 13q (see table) or gain of 15q, 18q or 20q (data not shown).
   C. List of small RNAs correlated with loss of X8p
   D. List of small RNAs correlated with loss of X17p
20. List of 23 best predictive small RNAs in adenoma vs control
21. Sequences of microarray probe inserts for small RNA detection
   22. Sample IDs of patient material
   23. Small RNA mature and star strand sequences
      A. Small RNA sequences derived from PCT/NL2007/000012
      B. Small RNAs sequences derived from public miRNA database miRBase
24. Small RNA precursor sequences
   A. Small RNA precursor sequences derived from PCT/NL2007/000012
   B. Small RNAs precursor sequences derived from public miRNA database miRBase
25. Classification on the basis of predictive small RNA subset for distinction between adenoma and adenocarcinoma
   A. List of small RNAs that are selected by PAM as good classifiers to distinguish colorectal adenoma from adenocarcinoma tissue
   B. Confusion tables before and after exclusion of the selected set of small RNAs
26 Most significantly differentially expressed small RNAs between adenomas and adenocarcinomas
   List of small RNAs (N=384) that are differentially expressed according to stringent statistical criteria (p<0.1). All of these small RNAs are downregulated in adenocarcinomas compared to adenomas (see also Fig. IIIc and IIId).
27 Selection of predictive small RNAs for classification of adenomas vs adenocarcinomas
   A selection of 91 small RNAs was made that can cluster adenomas or adenocarcinomas highly predictable (p<0.0001) (see also Fig IVa and IVb)
28 Small RNAs located on micro-deletions present in colorectal carcinomas
   Microdeletions of smaller than 3 megabases were determined in colorectal carcinomas using array CGH data.
   A, Small RNAs located in the determined deleted regions are listed.
   B, Differentially expressed small RNAs (adenoma vs carcinoma) localized in the deleted regions are listed
29 Small RNAs located on micro-gains present in colorectal carcinomas
   Microgains of smaller than 3 megabases were determined in colorectal carcinomas using array CGH data.
   A, Small RNAs located in the determined microgain regions are listed.
   B, Differentially expressed small RNAs (adenoma vs carcinoma) localized in the gained regions are listed
30 Small RNAs located on micro-deletion present in microsatellite stable colorectal carcinomas
   Microdeletions of smaller than 3 megabases were determined in colorectal microsatellite stable carcinomas using array CGH data. Small RNAs located in the determined deleted regions are listed.
31 Small RNAs located on micro-gains present in microsatellite stable colorectal carcinomas
   Microgains of smaller than 3 megabases were determined in colorectal microsatellite stable carcinomas using array CGH data. A, Small RNAs located in the determined microgain regions are listed. B, Differentially expressed small RNAs (adenoma vs adenocarcinoma) localized in the gained regions are listed
32 Small RNAs located on micro-deletions present in microsatellite instable colorectal carcinomas
   Microdeletions of smaller than 3 megabases were determined in colorectal microsatellite instable carcinomas using array CGH data.
   A, Small RNAs located in the determined deleted regions are listed.
   B, Differentially expressed small RNAs (adenoma vs adenocarcinoma) localized in the deleted regions are listed
33 Five small RNAs showed associated expression and DIVA copy number levels in colorectal cancer.
The software program ACE-it integrated small RNA based microarray data with array CGH data, determining whether differences in expression of small RNAs is a consequence of DNA copy number changes. Results show that expression of 5 small RNAs is associated with DNA copy number changes.
34 Presence of classifiers distinguishing adenomas from adenocarcinomas in faeces
   This table includes small RNAs that were selected as good classifiers in tables 25 and could be detected in faeces
35 Presence of classifiers distinguishing adenomas from adenocarcinomas in blood
   This table includes small RNAs that were selected as good classifiers in tables 25 and could be detected in blood
      The sequence of the small RNAs listed by name in the tables 1-35 is given in table 23. The sequence of associated precursors is given in table 24. The sequence of a collection of probes for the small RNAs of table 23 is given in table 21.

### BRIEF DESCRIPTION OF THE DRAWINGS

FIG. 1. a. Hierarchical cluster analysis of 36 colorectal adenoma samples and ten normal colorectal epithelium controls. Cluster analysis with expression data of 1554 small RNAs yields one cluster (cluster 1) containing six out of ten controls and a second cluster (cluster 2) containing 27 colorectal adenoma samples. Each column represents a colorectal (adenoma or control) tissue sample and each row represents a different small RNA. In the heat map, green signals represent down-regulation, black signals indicate no differences in expression of small RNAs and red signals represents up-regulation. Only a fraction of the complete heat chart is shown, indicating the clusters. b. Cross table adenomas and control samples versus cluster membership. Cluster 1 contains six controls and nine adenoma tumours and cluster 2 includes four control samples and 27 colorectal adenoma tumours. c and d. The same hierarchical clustering was performed on the basis of 852 small RNAs, that were differentially expressed with a p value<0.1 including a cross table. e and f. The same hierarchical clustering was performed on the basis of 359 small RNAs, that were differentially expressed with a p value<0.05 including a cross table. g. The same hierarchical clustering was performed on the basis of 230 small RNAs, that were differentially expressed with a p value<0.025. h. The same hierarchical clustering was performed on the basis of the best 23 predictive small RNAs.

FIG. 2. a. Hierarchical cluster analysis of 31 colorectal carcinomas samples and ten normal colorectal epithelium controls. Cluster analysis with expression data of 105 small RNAs yields one cluster (cluster 1) containing all ten control samples and a second cluster (cluster 2), including 26 colorectal carcinoma samples. Each column represents a colorectal (carcinoma or control) tissue sample and each row represents a different small RNA. In the heat map, green signals represent down-regulation, black signals indicate no differences in expression of small RNAs and red signals represents up-regulation. b. Cross table carcinomas and control samples versus cluster membership. Cluster 1 contains all ten control samples and five carcinoma tumours and cluster 2 includes 26 colorectal carcinomas. c and d. The same hierarchical clustering was performed on the basis of 44 small RNAs, that were differentially expressed with a p value<0.1 including a cross table.

FIG. 3. a. Hierarchical cluster analysis of 31 colorectal carcinomas samples and 36 colorectal adenomas. Cluster analysis with expression data of 1080 small RNAs yields one cluster (cluster 1) that includes 20 colorectal adenoma tumours and a second cluster (cluster 2), which comprises 24 of the colorectal carcinoma tumours. Each column represents a colorectal (carcinoma or adenoma) tissue sample and each row represents a different small RNA. In the heat map, green signals represent down-regulation, black signals indicate no differences in expression of small RNAs and red signals represents up-regulation. b. Cross table carcinomas and adenomas tumours samples versus cluster membership. Cluster 1 contains 20 colorectal adenoma tumours and seven colorectal carcinomas and cluster 2 contains 24 colorectal carcinomas and 16 colorectal adenoma samples. c and d. The same hierarchical clustering was performed on the basis of 384 small RNAs, that were differentially expressed with a p value<0.1 including a cross table.

FIG. 4. a. Hierarchical cluster analysis of 31 colorectal carcinomas samples and 36 colorectal adenomas. Cluster analysis with expression data of small RNAs yields one cluster (cluster 1) that includes 23 colorectal adenocarcinoma tumours and a second cluster (cluster 2), which comprises 28 of the colorectal adenoma tumours. Each column represents a colorectal (adenocarcinoma or adenoma) tissue sample and each row represents a different small RNA. In the heat map, green signals represent down-regulation, black signals indicate no differences in expression of small RNAs and red signals represents up-regulation. b. Cross table carcinomas and adenomas tumours samples versus cluster membership. Cluster 1 contains 8 colorectal adenoma tumours and 23 colorectal adenocarcinomas and cluster 2 contains 8 colorectal adenocarcinomas and 28 colorectal adenoma samples. The clustering is highly significant: p<0.0001.

FIG. 5. RT-PCR analysis of hsa-mir-24-2-1. RNA was isolated from 27 colorectal adenomas and 31 adenocarcinomas and Taqman PCR was performed. a, The expression levels of hsa-mir-24-2-1 RNA were detected and compared between the two groups. b, RT-PCR results were compared with expression level results for has-hir-24-2-1 as detected by microarray.

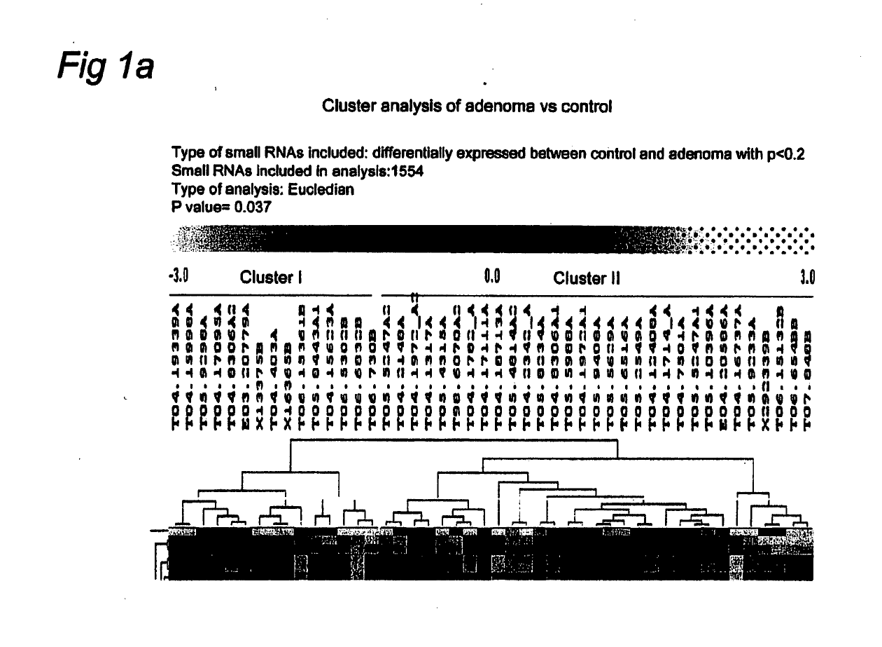

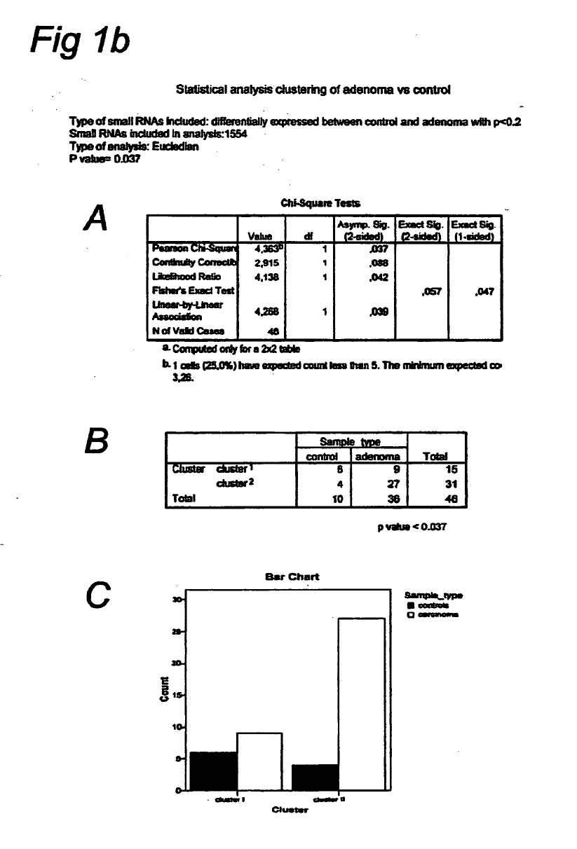

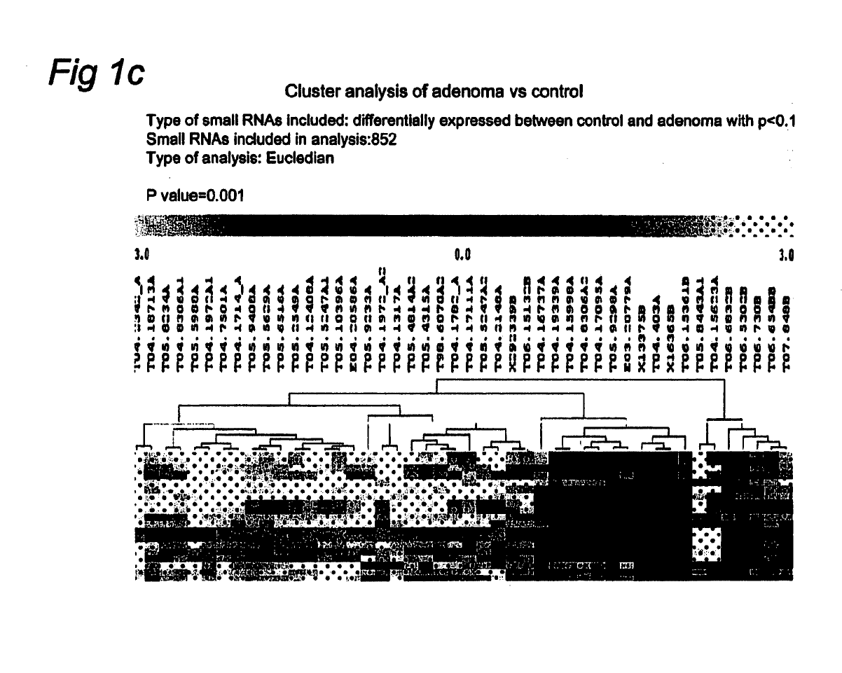

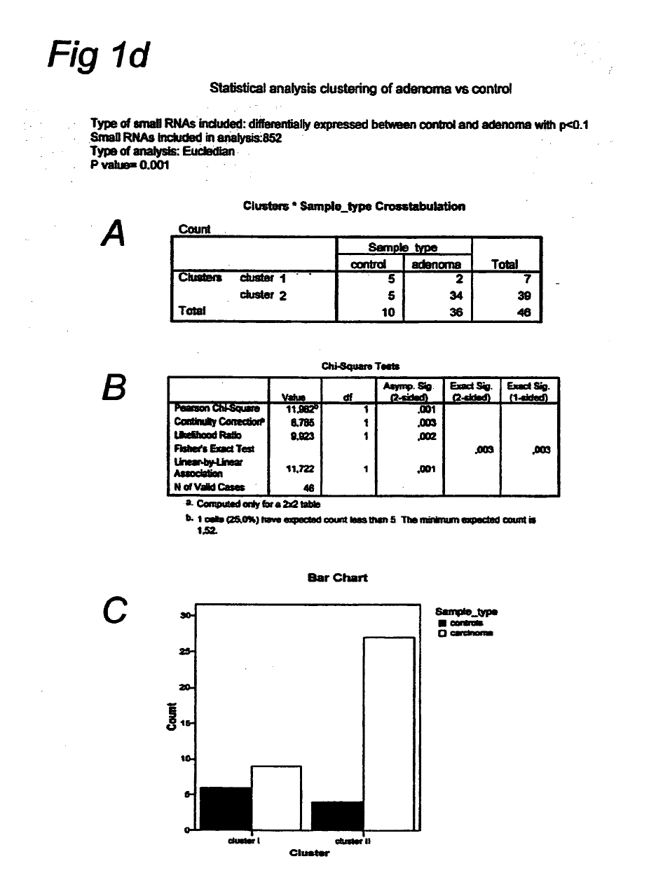

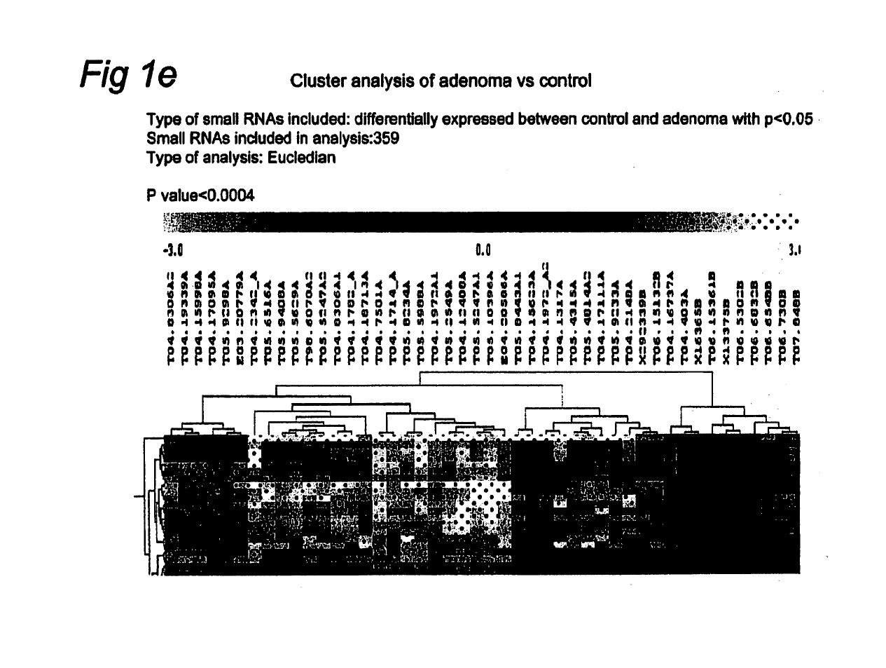

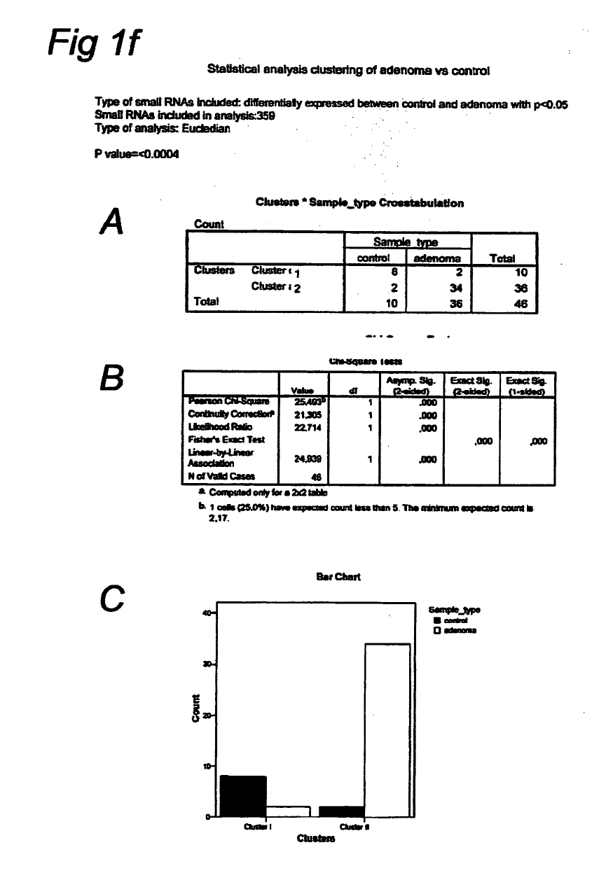

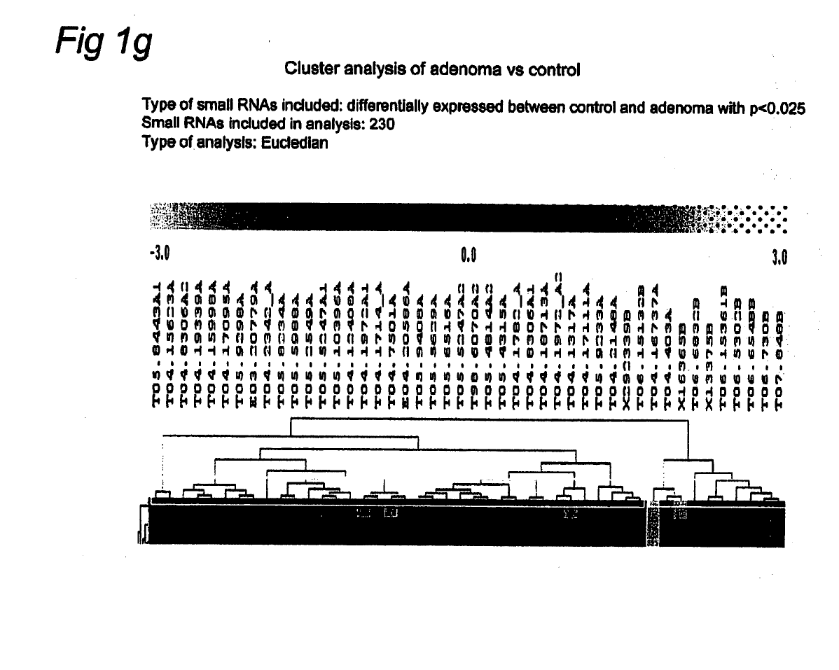

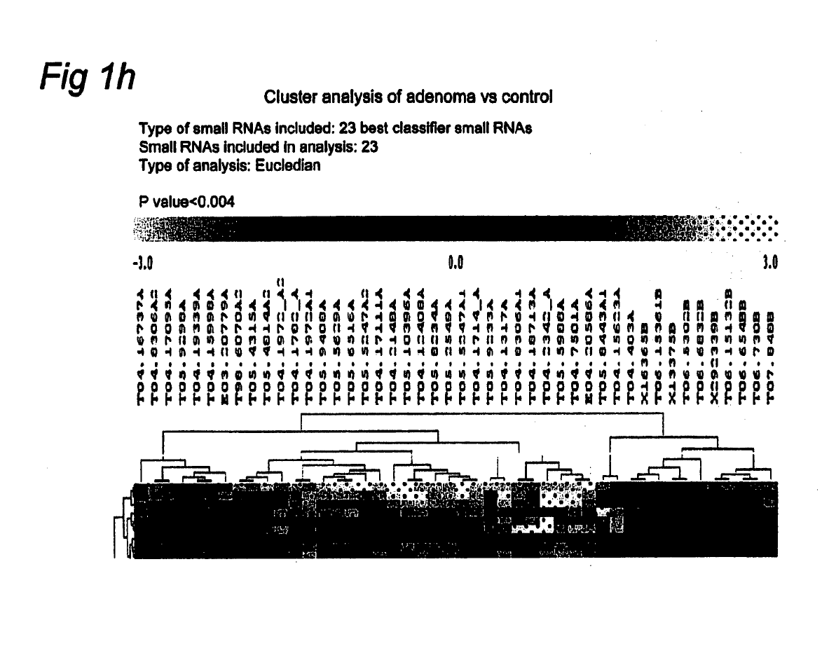

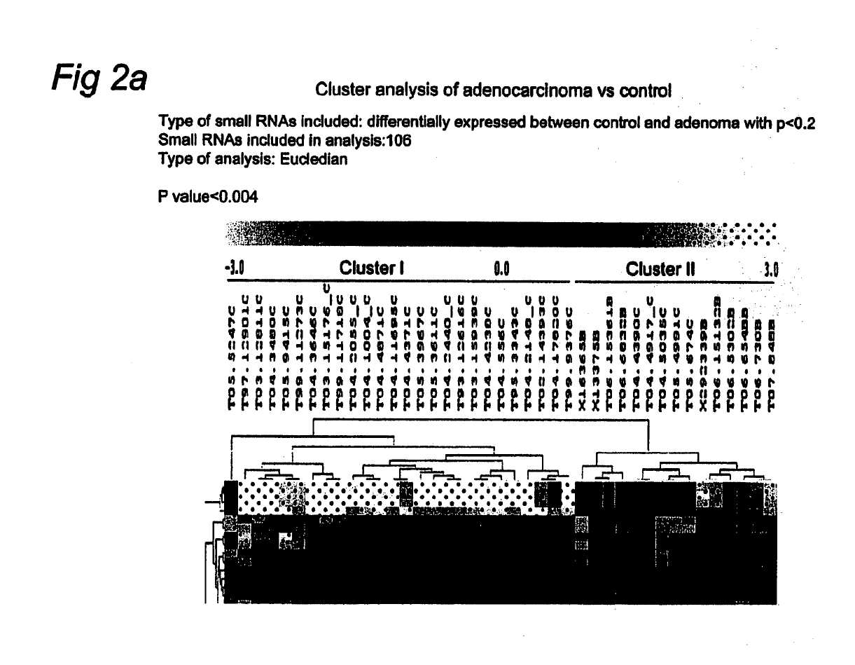

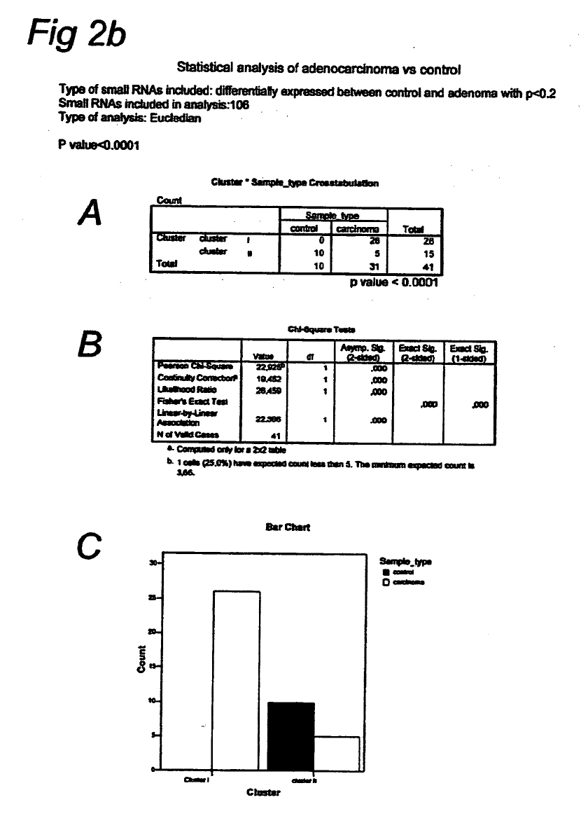

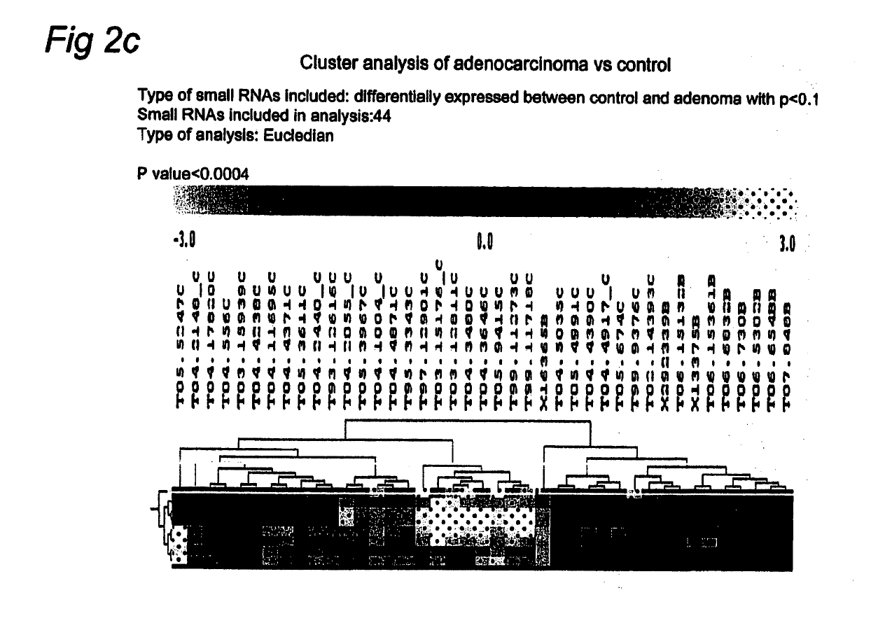

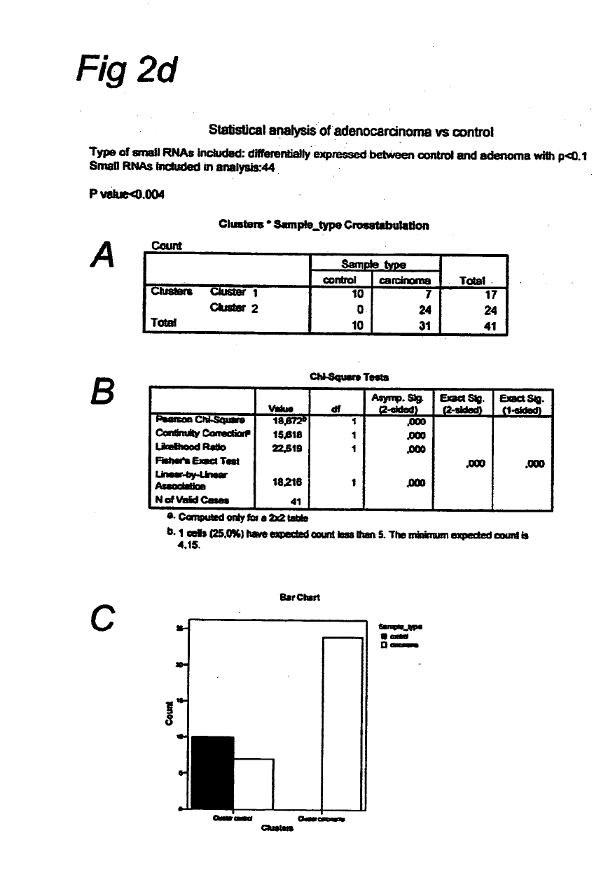

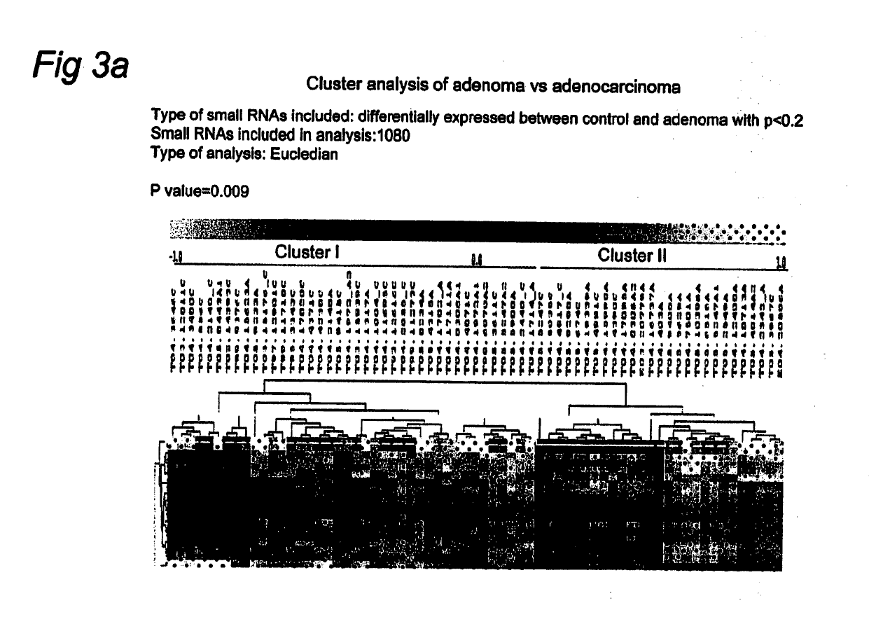

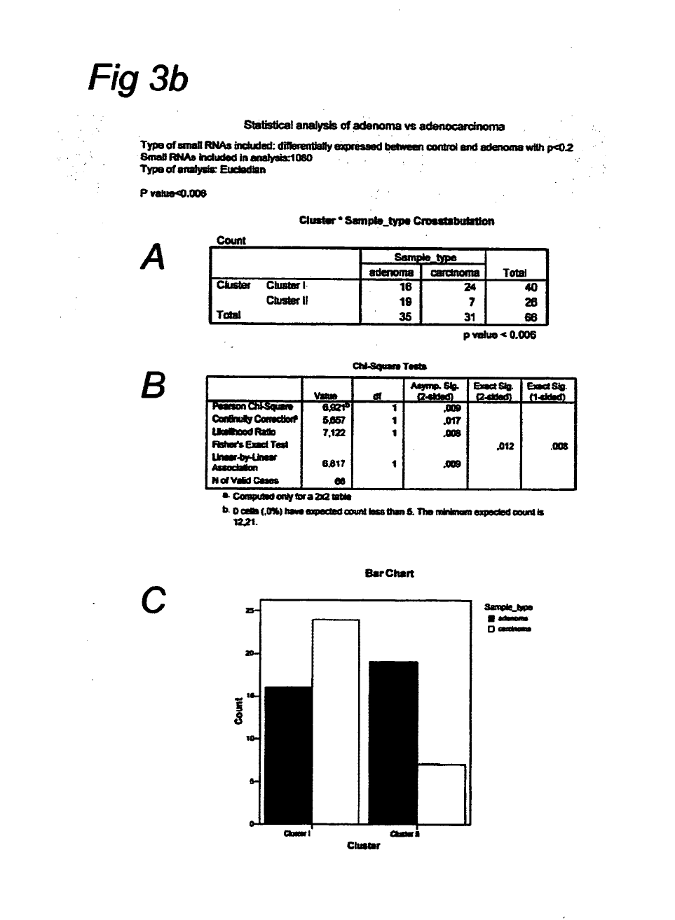

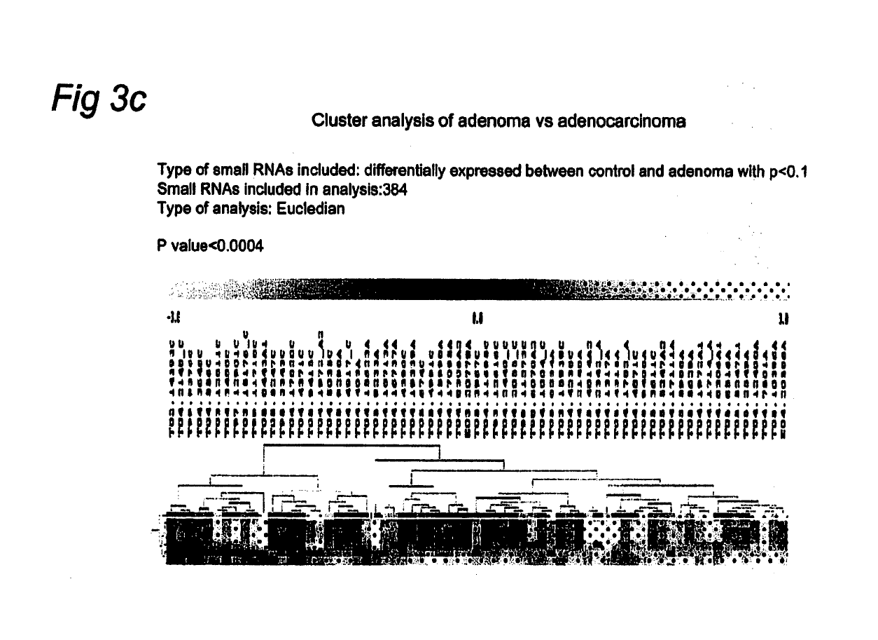

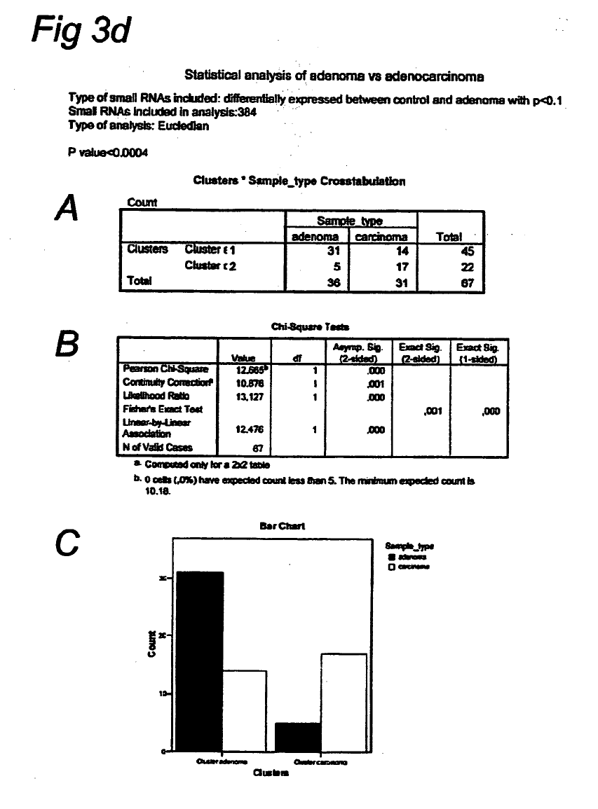

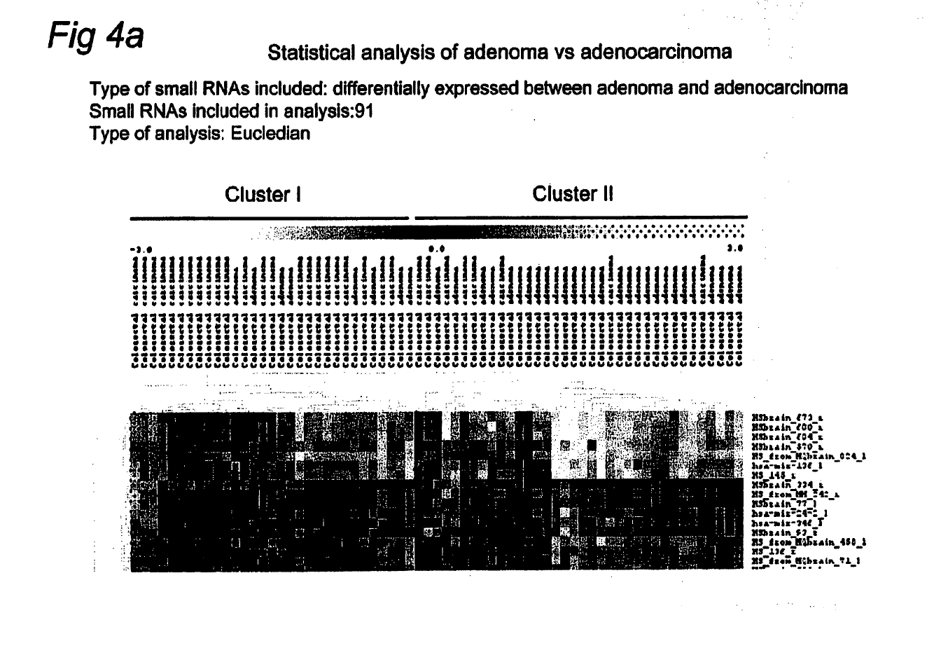

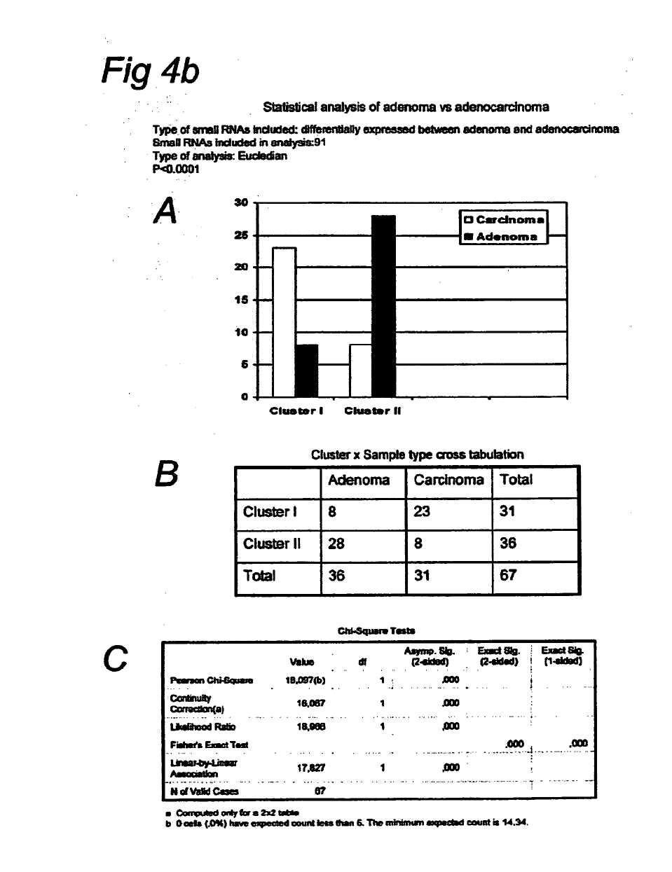

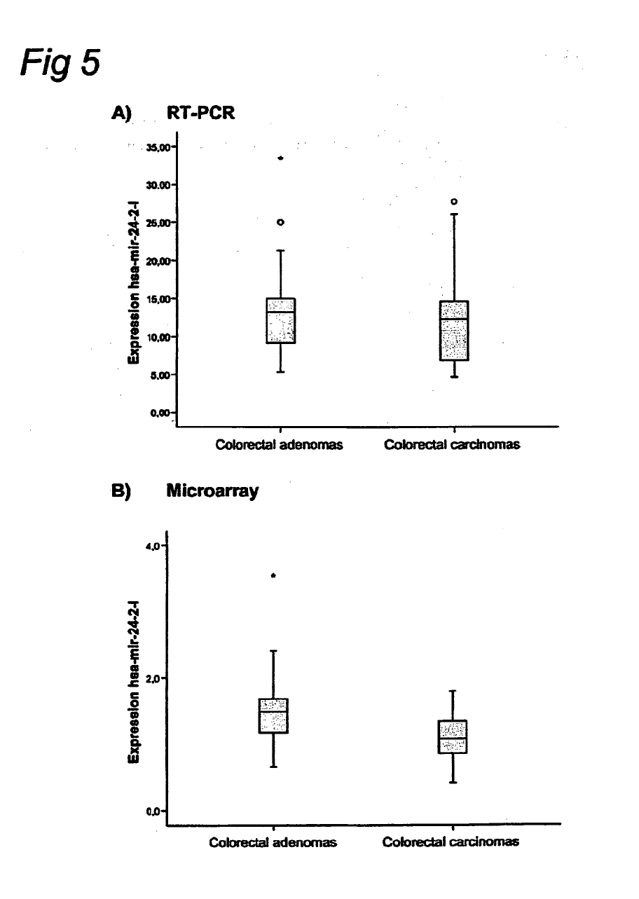

TABLE 1
Differentially expressed small RNAs in colorectal samples vs reference pool

| | MAX log2 | | |
|---|---|---|---|
| Systematic Name | (Sample/REF) | MIN log2 (Sample/REF) | log2 range¹ |
| Hsd_1_l | 2.088695405 | −1.912512559 | 4.001207964 |
| HS_from_MM_216_l | 3.039107456 | −0.964247181 | 4.003354637 |
| Hsd_95_r | 3.436602675 | −0.567767452 | 4.004370127 |
| hsa-mir-519b_r | 1.808824422 | −2.195595466 | 4.004419888 |
| HS_from_MQbrain_387_l | 3.011798091 | −0.99318349 | 4.004981581 |
| HS_from_MQbrain_1453_l | 3.18109327 | −0.826023558 | 4.007116828 |
| HS_146_l | 1.925545312 | −2.081708119 | 4.007253431 |
| Hsd_108_r | 1.995591703 | −2.013406769 | 4.008998471 |
| Hsd_33_r | 2.141519065 | −1.86783118 | 4.009350246 |
| HS_from_MQbrain_1851_r | 1.953336363 | −2.057525604 | 4.010861967 |
| HS_from_MQbrain_832_l | 3.446216492 | −0.566661909 | 4.012878401 |
| HSbrain_695_l | 1.63064494 | −2.383156365 | 4.013801306 |
| HS_from_MQbrain_285_r | 1.034414575 | −2.980175248 | 4.014589823 |
| HSbrain_741_r | 1.543845894 | −2.470976079 | 4.014821974 |
| HSbrain_421_l | 2.072907825 | −1.942291385 | 4.01519921 |
| HSbrain_649_r | 3.326270271 | −0.688997381 | 4.015267653 |
| Hsd_64_l | 1.408247542 | −2.608890571 | 4.017138113 |
| hsa-mir-526a-2_l | 2.708372776 | −1.309295741 | 4.017668517 |
| HS_from_MQbrain_782_l | 1.743725561 | −2.274212896 | 4.017938457 |
| HS_155_r | 2.837291129 | −1.181419902 | 4.018711031 |
| Hsd_61_l | 2.219038295 | −1.801406885 | 4.02044518 |
| HS_70_l | 2.52640836 | −1.494414894 | 4.020823253 |
| HSbrain_444_l | 1.582704817 | −2.438311287 | 4.021016104 |
| HS_from_MQbrain_1024_r | 3.014769503 | −1.007329753 | 4.022099256 |
| HSbrain_194_l | 2.348967735 | −1.673488376 | 4.022456112 |
| HS_from_PT_48_r | 2.631303094 | −1.39222147 | 4.023524564 |
| Hsd_from_Mmd_468_r | 2.454803712 | −1.568889086 | 4.023692798 |
| HSbrain_273_l | 1.831392763 | −2.192600976 | 4.023993739 |
| HS_74_l | 3.265905838 | −0.758224149 | 4.024129986 |
| HSbrain_524_l | 3.180726782 | −0.843561389 | 4.024288171 |
| HS_from_MM_299_r | 2.292740527 | −1.731689095 | 4.024429622 |
| HS_from_MQbrain_689_r | 2.222938714 | −1.802712043 | 4.025650757 |
| HS_from_MQbrain_1873_r | 2.237532145 | −1.788151021 | 4.025683166 |
| Hsd_from_Mmd_383_l | 3.448975628 | −0.577051179 | 4.026026807 |
| HS_from_MQbrain_222_l | 2.584403955 | −1.441772057 | 4.026176012 |
| Hsd_from_Mmd_237_r | 2.204167698 | −1.822293249 | 4.026460947 |
| HS_from_MQbrain_1332_r | 1.428609239 | −2.598212413 | 4.026821651 |
| hsa-mir-190_l | 0.962332185 | −3.064623649 | 4.026955834 |
| HS_from_MQbrain_345_l | 2.472953718 | −1.554177129 | 4.027130847 |
| Hsd_from_Mmd_306_r | 3.114214478 | −0.913664866 | 4.027879344 |
| HSbrain_1336_r | 1.98318273 | −2.044872431 | 4.028055161 |
| hsa-mir-590_r | 1.480590433 | −2.547657779 | 4.028248213 |
| Hsd_60_r | 2.714183878 | −1.314177354 | 4.028361232 |
| hsa-mir-527_l | 3.274510245 | −0.75478888 | 4.029299126 |
| hsa-mir-125b-1_l | 1.669667452 | −2.36030098 | 4.029968432 |
| HS_from_MM_208_l | 3.113520569 | −0.918925647 | 4.032446216 |
| Hsd_from_Mmd_42_r | 1.788514504 | −2.244615379 | 4.033129883 |
| HS_from_MQbrain_779_r | 3.074553118 | −0.95948886 | 4.034041979 |
| Hsd_97_l | 3.120215279 | −0.91479182 | 4.035007099 |
| HS_from_MQbrain_717_l | 3.336120523 | −0.700043063 | 4.036163585 |
| HS_from_MQbrain_995_l | 1.923042913 | −2.113284502 | 4.036327415 |
| HSbrain_379_l | 3.395930199 | −0.640690918 | 4.036621116 |
| HSbrain_360_r | 1.399949752 | −2.63714983 | 4.037099583 |
| HSbrain_23_r | 2.78275896 | −1.254450749 | 4.037209709 |
| HSbrain_46_r | 2.613865567 | −1.423787971 | 4.037653539 |
| hsa-mir-450-2_l | 0.985889776 | −3.051941859 | 4.037831636 |
| hsa-mir-299_r | 2.867791219 | −1.171901952 | 4.039693171 |
| Hsd_from_Mmd_41_l | 2.481504187 | −1.558977032 | 4.040481219 |
| HS_from_MQbrain_1734_l | 3.746423731 | −0.295131716 | 4.041555446 |
| HS_from_MQbrain_1182_r | 3.139834681 | −0.902030206 | 4.041864887 |
| HSbrain_665_r | 1.487643319 | −2.557027009 | 4.044670329 |
| HSbrain_563_l | 2.360039309 | −1.684782813 | 4.044822122 |
| HS_from_PT_292_l | 1.685434947 | −2.359996291 | 4.045431238 |
| HSbrain_182_l | 1.794410839 | −2.252022992 | 4.046433831 |
| HS_from_MQbrain_1197_l | 3.110590697 | −0.936448606 | 4.047039303 |
| Hsd_from_Mmd_49_r | 2.579386826 | −1.468504049 | 4.047890874 |
| hsa-mir-583_l | 2.230582983 | −1.820568126 | 4.051151108 |
| HS_298_r | 2.975580603 | −1.077341264 | 4.052921867 |
| HS_210_l | 3.005408513 | −1.049259575 | 4.054668089 |
| HS_from_MQbrain_1216_l | 2.724796118 | −1.330579318 | 4.055375436 |
| HS_from_MQbrain_1108_r | 2.862237119 | −1.193744151 | 4.05598127 |
| HSbrain_579_r | 1.40925375 | −2.647444316 | 4.056698066 |
| HS_from_MM_6_r | 3.124595142 | −0.933246204 | 4.057841346 |
| Hsd_from_Mmd_180_l | 1.610394113 | −2.447473763 | 4.057867876 |
| HS_from_MQbrain_1214_l | 2.82335625 | −1.23529739 | 4.05865364 |
| HS_from_MQbrain_1757_l | 3.146647396 | −0.912182423 | 4.058829818 |
| HS_from_MQbrain_984_r | 3.299222196 | −0.761264239 | 4.060486435 |
| Hsd_68_l | 3.407872364 | −0.652984039 | 4.060856403 |
| Hsd_from_Mmd_167_l | 3.307979739 | −0.755409571 | 4.06338931 |
| HS_from_PT_210_l | 1.555223227 | −2.508438651 | 4.063661879 |
| hsa-mir-29c_l | 2.251504167 | −1.812858532 | 4.064362698 |
| HS_from_MQbrain_946_l | 2.987652722 | −1.081392757 | 4.069045479 |
| Hsd_from_Mmd_336_l | 2.685058565 | −1.384237172 | 4.069295737 |
| HSbrain_11_l | 1.50304215 | −2.566901844 | 4.069943994 |
| HS_210_r | 2.548656471 | −1.521791374 | 4.070447845 |
| HS_from_MQbrain_766_r | 1.495004303 | −2.575468452 | 4.070472755 |
| HS_from_MQbrain_1504_l | 1.928779508 | −2.143286333 | 4.072065841 |
| HS_from_PT_221_l | 3.113947553 | −0.958652008 | 4.072599561 |
| HS_241_r | 2.727132873 | −1.345845446 | 4.072978319 |
| hsa-mir-450-1_l | 0.967359793 | −3.107519985 | 4.074879778 |
| HSbrain_2_r | 1.714101263 | −2.364273227 | 4.07837449 |
| HS_from_MM_112_r | 2.766784429 | −1.31191063 | 4.078695059 |
| hsa-mir-429_r | 3.270712052 | −0.808687162 | 4.079399213 |
| Hsd_from_Mmd_272_r | 2.686690588 | −1.39497378 | 4.081664368 |
| HSbrain_294_r | 1.93035153 | −2.156250764 | 4.086602294 |
| hsa-mir-525_r | 3.499359361 | −0.59153303 | 4.090892391 |
| hsa-mir-32_r | 1.511270971 | −2.580050206 | 4.091321177 |
| hsa-mir-194-2_l | 1.877987368 | −2.213953242 | 4.091940609 |
| HSbrain_601_l | 1.447126351 | −2.645387932 | 4.092514283 |
| hsa-mir-146b_l | 1.231334378 | −2.862293453 | 4.093627831 |
| Hsd_from_Mmd_518_r | 2.983229897 | −1.110549566 | 4.093779463 |
| HS_from_MQbrain_744_l | 1.996266499 | −2.099304092 | 4.095570591 |
| HS_from_MQbrain_613_r | 3.331023916 | −0.764672498 | 4.095696414 |
| HS_from_MQbrain_1663_r | 1.446639428 | −2.649696845 | 4.096336273 |
| HSbrain_750_l | 2.08655999 | −2.011722123 | 4.098282113 |
| hsa-mir-194-1_l | 1.930819662 | −2.168480826 | 4.099300489 |
| HS_from_MQbrain_740_l | 2.298572839 | −1.801408165 | 4.099981003 |
| HS_232_r | 1.212775317 | −2.888763224 | 4.10153854 |
| Hsd_from_Mmd_413_l | 3.254011952 | −0.847828032 | 4.101839983 |
| HS_242_l | 3.281061296 | −0.821383269 | 4.102444565 |
| hsa-mir-302d_r | 1.134037131 | −2.969490448 | 4.103527579 |
| HS_13_r | 3.325827789 | −0.778265765 | 4.104093555 |
| HSbrain_33_r | 2.646811644 | −1.458438688 | 4.105250333 |
| Hsd_66_l | 2.387342505 | −1.717921714 | 4.10526422 |
| hsa-mir-639_r | 3.362748936 | −0.742532587 | 4.105281523 |
| HS_from_MQbrain_1048_r | 3.368346417 | −0.736954975 | 4.105301392 |
| hsa-mir-630_l | 0.934157367 | −3.172776941 | 4.106934308 |
| HS_from_MQbrain_21_l | 1.739970275 | −2.367317237 | 4.107287512 |
| HSbrain_127_r | 0.842157726 | −3.267834226 | 4.109991953 |
| Hsd_from_Mmd_69_l | 3.35164685 | −0.758839345 | 4.110486195 |
| HS_from_MQbrain_198_l | 3.019877358 | −1.090753169 | 4.110630528 |
| HS_from_MQbrain_198_l | 2.446960973 | −1.66685195 | 4.113812923 |
| HS_from_MQbrain_663_l | 1.257822727 | −2.857700652 | 4.115523379 |
| HSbrain_1387_l | 2.551619155 | −1.564398693 | 4.116017848 |
| hsa-mir-486_r | 2.253616104 | −1.862596947 | 4.116213052 |
| hsa-mir-424_l | 1.874062513 | −2.242250797 | 4.116313311 |
| HSbrain_566_r | 3.063293636 | −1.053635725 | 4.116929361 |
| HSbrain_1392_l | 2.525149812 | −1.591990324 | 4.117140135 |
| HSbrain_100_r | 3.126583282 | −0.990797952 | 4.117381235 |
| HS_from_PT_108_l | 3.792884876 | −0.325020225 | 4.117905101 |
| HS_from_MQbrain_261_r | 2.72514481 | −1.394675248 | 4.119820057 |
| HS_from_MQbrain_549_r | 2.403950881 | −1.716060047 | 4.120010928 |
| HSbrain_668_r | 3.09183027 | −1.028865429 | 4.120695699 |
| HSbrain_125_l | 1.812802385 | −2.308656215 | 4.1214586 |
| HSbrain_815_l | 1.494998806 | −2.627703969 | 4.122702775 |
| HS_from_MQbrain_366_l | 1.873826288 | −2.249669571 | 4.123495859 |
| HS_from_MQbrain_1306_l | 3.481994339 | −0.642164797 | 4.124159135 |
| HS_from_MQbrain_637_l | 3.269301702 | −0.855197558 | 4.12449926 |
| HS_from_MQbrain_1055_l | 3.826461999 | −0.298321824 | 4.124783823 |
| hsa-mir-182_l | 3.043662609 | −1.082124344 | 4.125786953 |
| HS_from_PT_134_r | 1.677604376 | −2.448920174 | 4.12652455 |
| HS_from_MQbrain_283_l | 1.511361471 | −2.615228726 | 4.126590197 |
| HSbrain_421_r | 2.294092029 | −1.833066173 | 4.127158202 |
| hsa-mir-606_r | 1.673921879 | −2.454731269 | 4.128653148 |
| HS_from_MM_123_r | 2.41352431 | −1.71625623 | 4.12978054 |
| HSbrain_670_l | 2.568284445 | −1.56212134 | 4.130405785 |
| HS_from_MQbrain_1112_l | 2.498183737 | −1.634144642 | 4.13232838 |
| Hsd_56_l | 2.102749876 | −2.030606037 | 4.133355913 |
| HS_from_MQbrain_1682_r | 2.090005371 | −2.044755865 | 4.134761237 |
| HS_from_MQbrain_663_r | 2.865084367 | −1.269689425 | 4.134773792 |
| HS_269_r | 3.826634218 | −0.309441006 | 4.136075224 |
| HSbrain_341_r | 1.858468781 | −2.279907256 | 4.138376036 |
| HS_from_MQbrain_1779_l | 2.793138989 | −1.346080788 | 4.139219777 |
| HSbrain_491_l | 1.302048165 | −2.837651231 | 4.139699395 |
| hsa-mir-98_l | 0.966572026 | −3.173625248 | 4.140197274 |
| HS_from_PT_70_l | 1.059894906 | −3.082005806 | 4.141900712 |
| HS_from_MQbrain_1656_r | 2.347722084 | −1.797177196 | 4.14489928 |
| HS_from_MQbrain_1410_l | 2.1604767 | −1.984558858 | 4.145035557 |
| HS_203_r | 1.993454149 | −2.15178467 | 4.14523882 |
| HSbrain_274_r | 2.551357842 | −1.595269122 | 4.146626965 |
| HSbrain_848_l | 2.826198231 | −1.320514298 | 4.146712528 |
| Hsd_from_Mmd_326_r | 2.412744392 | −1.735454736 | 4.148199128 |
| HSbrain_561_r | 4.051652073 | −0.099717017 | 4.151369091 |
| HS_from_MQbrain_1235_l | 3.416522189 | −0.735166181 | 4.15168837 |
| HSbrain_439_l | 1.289303857 | −2.862525771 | 4.151829629 |
| HS_from_MQbrain_1711_l | 3.497043706 | −0.655263539 | 4.152307245 |
| hsa-mir-653_r | 0.98182448 | −3.171621167 | 4.153445647 |
| HS_from_MM_44_r | 2.804739701 | −1.349347414 | 4.154087115 |
| HSbrain_595_l | 1.463193402 | −2.694308006 | 4.157501407 |
| HS_from_MQbrain_545_l | 3.22663762 | −0.931256531 | 4.15789415 |
| HS_249_r | 1.514697456 | −2.644437886 | 4.159135342 |
| HSbrain_64_r | 1.971703045 | −2.187896901 | 4.159599947 |
| HS_from_MQbrain_952_l | 3.446889258 | −0.718850097 | 4.165739355 |
| HSbrain_486_r | 1.897118787 | −2.268757685 | 4.165876473 |
| hsa-mir-675_l | 3.115834412 | −1.051639823 | 4.167474235 |
| HS_from_MM_339_l | 1.597557716 | −2.571789523 | 4.169347238 |
| HSbrain_344_r | 1.467181102 | −2.703440434 | 4.170621536 |
| HS_from_MQbrain_1525_r | 2.789776074 | −1.381436483 | 4.171212557 |
| HS_from_MQbrain_1880_l | 3.193429908 | −0.978119046 | 4.171548954 |
| HSbrain_259_l | 3.182274869 | −0.989342132 | 4.171617001 |
| HS_36_l | 2.048680384 | −2.124225689 | 4.172906073 |
| HSbrain_1398_l | 2.411483784 | −1.761492961 | 4.172976745 |
| hsa-mir-504_l | 3.725684236 | −0.449577893 | 4.175262129 |
| HSbrain_490_r | 2.474326459 | −1.702004657 | 4.176331116 |
| hsa-mir-518a-1_l | 3.217992148 | −0.958678037 | 4.176670184 |
| hsa-mir-502_r | 3.042089261 | −1.135977424 | 4.178066684 |
| HS_from_MQbrain_1760_l | 2.670972591 | −1.508208772 | 4.179181363 |
| HS_from_MQbrain_1685_r | 2.747861927 | −1.433994718 | 4.181856645 |
| HS_51_r | 0.981611798 | −3.202059726 | 4.183671524 |
| HS_from_MQbrain_638_l | 1.253620613 | −2.931319081 | 4.184939694 |
| Hsd_68_l | 2.193173751 | −1.991888607 | 4.185062359 |
| hsa-mir-34b_r | 1.448020386 | −2.737588019 | 4.185608405 |
| hsa-mir-575_l | 3.249553651 | −0.937042648 | 4.186596299 |
| HSbrain_645_r | 2.889118823 | −1.29773156 | 4.186850383 |
| HS_80_r | 3.559275497 | −0.627597731 | 4.186873227 |
| hsa-mir-1-2_r | 1.726687457 | −2.460219642 | 4.186907098 |
| HSbrain_240_r | 1.808684952 | −2.378448037 | 4.187132989 |
| HS_from_MQbrain_1261_l | 0.989075555 | −3.198383282 | 4.187458837 |
| HSbrain_209_r | 2.441264501 | −1.746557031 | 4.187821532 |
| Hsd_from_Mmd_54_r | 2.481322129 | −1.707213145 | 4.188535274 |
| HS_from_MQbrain_31_l | 3.528457094 | −0.660289124 | 4.188746218 |
| HS_248_r | 1.565168322 | −2.624393107 | 4.189561428 |
| HSbrain_88_r | 2.495129206 | −1.695239284 | 4.19036849 |
| HS_from_MQbrain_297_l | 1.937994705 | −2.252672464 | 4.190667169 |
| HSbrain_682_r | 1.410233792 | −2.780493784 | 4.190727576 |
| HS_from_MQbrain_1474_r | 2.129160072 | −2.063207402 | 4.192367474 |
| Hsd_from_Mmd_184_r | 1.692567998 | −2.499800673 | 4.192368671 |
| hsa-mir-148a_r | 2.381580191 | −1.811256667 | 4.192836858 |
| HS_from_MQbrain_1325_r | 3.40225604 | −0.79135648 | 4.19361252 |
| HSbrain_414_l | 1.257763203 | −2.937220384 | 4.194983586 |
| HS_from_MM_236_l | 3.202704643 | −0.99362294 | 4.196327582 |
| HS_from_MQbrain_634_l | 1.69303102 | −2.504197611 | 4.197228631 |
| HS_from_MQbrain_572_l | 1.50074111 | −2.701387364 | 4.202128474 |
| hsa-mir-99a_l | 1.502009642 | −2.700136694 | 4.202146337 |
| HS_from_PT_157_l | 2.462685253 | −1.740216562 | 4.202901815 |
| HSbrain_1391_l | 2.671096297 | −1.532233245 | 4.203329541 |
| HS_from_MQbrain_1778_r | 2.934787151 | −1.26895031 | 4.203737461 |
| HS_161_l | 1.674576671 | −2.530254246 | 4.204830917 |
| HS_from_MQbrain_1985_l | 3.558937263 | −0.646414954 | 4.205352217 |
| HSbrain_354_r | 3.215840235 | −0.989522007 | 4.205362242 |
| HSbrain_358_l | 3.050110083 | −1.156063694 | 4.206173777 |
| HSbrain_1408_l | 1.59049657 | −2.616103125 | 4.206599695 |
| HS_from_MQbrain_135_r | 2.52167077 | −1.686270916 | 4.207941686 |
| HS_from_MQbrain_1_l | 2.618807889 | −1.590013898 | 4.208821787 |
| HSbrain_280_l | 2.400542218 | −1.811399359 | 4.211941577 |
| HSbrain_810_r | 1.526197944 | −2.687063483 | 4.213261427 |
| HS_from_MQbrain_1217_l | 2.951744954 | −1.261942862 | 4.213687816 |
| HS_from_MM_178_l | 2.777706709 | −1.437120908 | 4.214827616 |
| HSbrain_737_r | 2.512564121 | −1.704063393 | 4.216627514 |
| HSbrain_552_l | 2.860555433 | −1.356572784 | 4.217128217 |
| HS_from_MQbrain_820_r | 1.545176254 | −2.672133591 | 4.217309845 |
| Hsd_from_Mmd_81_l | 2.472179995 | −1.74637033 | 4.218550325 |
| HS_from_MQbrain_1756_r | 1.681853948 | −2.536721437 | 4.218575385 |
| HS_from_MQbrain_1195_r | 1.885039255 | −2.333704713 | 4.218743968 |
| HS_from_MM_194_r | 3.005858693 | −1.213331908 | 4.219190601 |
| Hsd_from_Mmd_326_l | 1.716092716 | −2.503314163 | 4.219406879 |
| HS_from_MQbrain_63_r | 1.519160307 | −2.702599207 | 4.221759513 |
| HS_from_MQbrain_1849_l | 3.839096883 | −0.382995879 | 4.222092763 |
| HS_from_PT_294_l | 2.934016726 | −1.289998538 | 4.224015264 |
| hsa-mir-635_r | 0.928797317 | −3.296314912 | 4.225112229 |
| HSbrain_736_r | 2.710837276 | −1.514896277 | 4.225733554 |
| Hsd_from_Mmd_266_l | 1.401547647 | −2.825134496 | 4.226682143 |
| HSbrain_172_l | 3.029809819 | −1.196882004 | 4.226691823 |
| Hsd_from_Mmd_461_r | 3.638050804 | −0.589232538 | 4.227283342 |
| HS_from_MQbrain_450_l | 1.868517764 | −2.35909134 | 4.227609104 |
| HSbrain_436_r | 2.923867369 | −1.303801843 | 4.227669212 |
| Hsd_from_Mmd_464_l | 2.308890741 | −1.920451642 | 4.229342384 |
| hsa-mir-205_l | 3.176538808 | −1.057279883 | 4.233818691 |
| HS_from_MQbrain_484_l | 2.916214688 | −1.317725066 | 4.233939754 |
| Hsd_from_Mmd_225_r | 2.907514253 | −1.327273348 | 4.234787601 |
| HSbrain_616_l | 3.607147454 | −0.628332803 | 4.235480257 |
| HSbrain_680_r | 1.472380281 | −2.763715095 | 4.236095376 |
| Hsd_39_r | 3.060942071 | −1.175793781 | 4.236735853 |
| HSbrain_612_r | 3.328984449 | −0.908882357 | 4.237866806 |
| HS_from_MM_294_l | 2.811442111 | −1.426899879 | 4.238341991 |
| HS_80_l | 3.517733101 | −0.725042388 | 4.242775489 |
| HS_from_MM_310_r | 2.782446733 | −1.460597803 | 4.243044536 |
| HS_from_PT_176_r | 3.245762647 | −1.000908258 | 4.246670905 |
| HS_from_MQbrain_799_l | 3.595960369 | −0.651540194 | 4.247500563 |
| HS_86_l | 2.379766914 | −1.868806993 | 4.248573906 |
| HS_from_MQbrain_1885_l | 1.245427632 | −3.005778939 | 4.251206571 |
| HSbrain_76_r | 1.50817019 | −2.746517086 | 4.254687277 |
| HS_from_MQbrain_1396_l | 2.443623064 | −1.812361766 | 4.25598483 |
| HS_from_MQbrain_869_r | 3.610900505 | −0.645668315 | 4.256568821 |
| hsa-mir-302a_r | 1.230869052 | −3.027608594 | 4.258477647 |
| HS_from_MQbrain_1300_l | 3.768112955 | −0.490898468 | 4.259011423 |
| HSbrain_537_l | 2.13090412 | −2.128153839 | 4.259057958 |
| HS_from_MQbrain_1856_l | 3.301347997 | −0.958010544 | 4.25935854 |
| HSbrain_674_l | 2.805619786 | −1.455808413 | 4.261428199 |
| Hsd_109_l | 2.223976209 | −2.039753844 | 4.263730053 |
| HS_from_MQbrain_1489_l | 3.112857587 | −1.150941421 | 4.263799007 |
| HS_from_MQbrain_579_l | 3.413818624 | −0.850427918 | 4.264246542 |
| HS_303_l | 1.686349724 | −2.579990869 | 4.266340593 |
| HS_from_MQbrain_150_r | 1.317629296 | −2.949323637 | 4.266952934 |
| HSbrain_485_l | 2.718807568 | −1.548574948 | 4.267382515 |
| HS_from_MQbrain_68_r | 2.204805375 | −2.063481157 | 4.268286531 |
| hsa-mir-199a-2_r | 1.898521955 | −2.371665771 | 4.270187726 |
| HSbrain_629_r | 1.551897427 | −2.718549113 | 4.27044654 |
| Hsd_from_Mmd_263_r | 2.9987514 | −1.273122535 | 4.271873935 |
| HS_from_MQbrain_1387_l | 2.950511517 | −1.323862732 | 4.274374249 |
| hsa-mir-518a-2_l | 3.541635836 | −0.737015883 | 4.278651719 |
| HS_156_r | 2.35772661 | −1.922889953 | 4.280616563 |
| HS_from_MQbrain_1322_r | 2.965520841 | −1.317796304 | 4.283317145 |
| HSbrain_37_r | 1.417208531 | −2.868417211 | 4.285625742 |
| Hsd_78_l | 2.638054745 | −1.64824861 | 4.286303355 |
| HS_207_r | 3.680232978 | −0.60794586 | 4.288178838 |
| Hsd_from_Mmd_23_r | 1.292673035 | −2.996174822 | 4.288847858 |
| HS_from_MQbrain_106_l | 1.942789396 | −2.34615461 | 4.288944006 |
| HS_from_MQbrain_1345_l | 2.59664488 | −1.693143144 | 4.289788024 |
| HSbrain_684_r | 1.465370969 | −2.82459941 | 4.289970379 |
| HS_from_PT_118_r | 3.968735645 | −0.321653838 | 4.290389483 |
| HSbrain_120_l | 3.00499768 | −1.287502149 | 4.292499829 |
| HSbrain_315_r | 1.472305455 | −2.821264784 | 4.293570239 |
| HSbrain_287_r | 2.849961269 | −1.444781236 | 4.294742505 |
| hsa-mir-107_r | 1.993410686 | −2.3019042 | 4.295314886 |
| Hsd_35_r | 3.230763888 | −1.065574345 | 4.296338233 |
| HS_247_r | 1.560942652 | −2.735473232 | 4.296415884 |
| HS_from_MQbrain_1746_l | 3.111397024 | −1.185339352 | 4.296736376 |
| HS_from_MQbrain_578_l | 2.764554074 | −1.532398648 | 4.296952723 |
| HS_from_PT_301_r | 2.180273215 | −2.1170404 | 4.297313615 |
| HS_from_MQbrain_836_l | 2.785167893 | −1.514221257 | 4.29938915 |
| HSbrain_869_l | 1.687597965 | −2.61309708 | 4.300695045 |
| HSbrain_209_l | 3.548747368 | −0.755661606 | 4.304408974 |
| HS_from_PT_211_l | 2.902527183 | −1.403507999 | 4.306035182 |
| HS_from_MQbrain_451_r | 1.019967237 | −3.286548425 | 4.306515662 |
| HSbrain_1331_r | 3.579692118 | −0.727316755 | 4.307008873 |
| Hsd_59_l | 2.691558346 | −1.616238627 | 4.307796973 |
| HS_from_MQbrain_339_l | 3.305410559 | −1.003192418 | 4.308602977 |
| HS_from_MQbrain_1522_l | 2.800381489 | −1.510695647 | 4.311077135 |
| HS_from_MQbrain_385_l | 3.237637453 | −1.073728609 | 4.311366062 |
| HS_55_l | 1.722065182 | −2.590618285 | 4.312683467 |
| HS_from_MQbrain_635_l | 1.658237403 | −2.656390086 | 4.314627489 |
| hsa-mir-320_r | 2.706082085 | −1.609750237 | 4.315832323 |
| HSbrain_673_r | 1.501956895 | −2.814291249 | 4.316248144 |
| HS_from_MQbrain_91_l | 2.363727935 | −1.952949293 | 4.316677228 |
| HS_from_MQbrain_80_l | 2.864297039 | −1.453562797 | 4.317859836 |
| HS_from_MQbrain_281_r | 1.76433421 | −2.554908026 | 4.319242236 |
| HS_from_MQbrain_932_l | 1.614404263 | −2.704963908 | 4.319368171 |
| HS_from_MQbrain_778_r | 3.243268583 | −1.076769934 | 4.320038517 |
| Hsd_from_Mmd_293_l | 1.530646274 | −2.789430607 | 4.320076881 |
| HSbrain_492_r | 3.37112138 | −0.950022151 | 4.321143531 |
| HS_from_MQbrain_904_r | 3.01547216 | −1.306889582 | 4.322361741 |
| HSbrain_134_l | 2.913704299 | −1.410680816 | 4.324385114 |
| HS_from_PT_55_r | 1.702808701 | −2.621922146 | 4.324730846 |
| HSbrain_630_r | 1.437649162 | −2.88773263 | 4.325381792 |
| Hsd_from_Mmd_382_r | 4.061472311 | −0.265165071 | 4.326637382 |
| HS_from_MQbrain_398_l | 3.327522246 | −0.999576342 | 4.327098587 |
| hsa-mir-574_l | 1.800159605 | −2.527710871 | 4.327870476 |
| HS_from_MM_334_l | 2.441453922 | −1.886565596 | 4.328019518 |
| HS_246_r | 1.645801886 | −2.683296681 | 4.329098567 |
| Hsd_from_Mmd_515_r | 3.418010274 | −0.912720525 | 4.3307308 |
| hsa-mir-145_r | 2.776275383 | −1.554693859 | 4.330969242 |
| HS_from_MQbrain_244_l | 2.847057079 | −1.487503511 | 4.33456059 |
| HS_from_MQbrain_1327_r | 3.716135032 | −0.620138116 | 4.336273147 |
| hsa-mir-146a_l | 1.751434667 | −2.585843672 | 4.337278339 |
| HS_from_MQbrain_284_l | 3.546036194 | −0.793745822 | 4.339782016 |
| HS_from_MQbrain_1553_l | 1.638201508 | −2.702270891 | 4.340472399 |
| HSbrain_620_r | 2.145542525 | −2.19517459 | 4.340717115 |
| hsa-mir-30a_l | 1.356214192 | −2.984587855 | 4.340802047 |
| hsa-mir-302b_l | 2.93276614 | −1.411032981 | 4.343799121 |
| HSbrain_688_r | 3.114665698 | −1.230112179 | 4.344777877 |
| HSbrain_363_r | 2.121405211 | −2.225401911 | 4.346807122 |
| HSbrain_666_l | 2.562979559 | −1.784301651 | 4.34728121 |
| HS_from_MQbrain_922_r | 2.658152261 | −1.689314118 | 4.347466378 |
| Hsd_79_r | 3.259391947 | −1.089850572 | 4.349242518 |
| HS_from_MQbrain_954_r | 1.630346726 | −2.723582546 | 4.353929272 |
| HSbrain_677_r | 1.495676583 | −2.859143772 | 4.354820355 |
| HS_from_MQbrain_1670_r | 2.754544435 | −1.600565308 | 4.355109743 |
| HSbrain_669_r | 1.430328027 | −2.925989464 | 4.356317492 |
| hsa-mir-520d_r | 2.847548728 | −1.509966377 | 4.357515105 |
| HSbrain_519_l | 3.691255539 | −0.666768683 | 4.358024222 |
| HSbrain_1388_l | 2.716374763 | −1.643334542 | 4.359709305 |
| HS_from_PT_95_l | 1.73042541 | −2.630740693 | 4.361166103 |
| HS_179_r | 1.614599929 | −2.748003316 | 4.362603246 |
| hsa-mir-451_l | 2.184012027 | −2.178954894 | 4.362966922 |
| HS_from_MQbrain_741_l | 1.394403683 | −2.968605749 | 4.363009431 |
| HS_from_MQbrain_1424_r | 1.578189632 | −2.786536503 | 4.364726135 |
| Hsd_111_l | 2.177414337 | −2.187565567 | 4.364979904 |
| HSbrain_477_l | 3.356586104 | −1.00848162 | 4.365067723 |
| HS_277_r | 1.616373868 | −2.756902974 | 4.373276842 |
| HSbrain_198_l | 1.514081575 | −2.864075011 | 4.378156586 |
| HS_from_MQbrain_54_r | 2.069520645 | −2.310045271 | 4.379565915 |
| HS_from_MM_299_l | 1.877993675 | −2.503341883 | 4.381335558 |
| Hsd_from_Mmd_181_l | 4.044579996 | −0.339367355 | 4.383947351 |
| HS_from_MQbrain_1721_r | 3.267494953 | −1.11686394 | 4.384358893 |
| HS_17_r | 2.958938895 | −1.431731886 | 4.390670781 |
| HSbrain_357_r | 1.88320241 | −2.508549736 | 4.391752146 |
| HS_from_MQbrain_1815_l | 1.863856722 | −2.528287591 | 4.392144313 |
| HS_from_MQbrain_115_l | 1.73172028 | −2.660450811 | 4.39217109 |
| HS_from_MM_264_l | 3.187804117 | −1.204639433 | 4.39244355 |
| HS_from_PT_99_l | 4.187204332 | −0.207565199 | 4.39476953 |
| HS_from_MQbrain_499_r | 2.915614281 | −1.479420493 | 4.395034773 |
| HS_from_MQbrain_476_r | 3.652782317 | −0.743903218 | 4.396685535 |
| hsa-mir-15b_l | 1.171609006 | −3.225080876 | 4.396689881 |
| HSbrain_592_r | 3.12628873 | −1.27307126 | 4.39935999 |
| Hsd_56_l | 3.329760494 | −1.072669427 | 4.402429922 |
| HS_from_MQbrain_1322_l | 2.298050317 | −2.105139368 | 4.403189685 |
| HSbrain_656_l | 2.995302493 | −1.408744426 | 4.404046919 |
| Hsd_from_Mmd_435_r | 1.965877521 | −2.438930551 | 4.404808071 |
| HS_14_r | 2.279108528 | −2.127372705 | 4.406481233 |
| HS_from_MM_70_l | 1.470797877 | −2.93634624 | 4.407144117 |
| HS_from_MQbrain_113_l | 3.305005418 | −1.10233229 | 4.407337709 |
| HSbrain_65_l | 2.857530515 | −1.550065162 | 4.407595677 |
| Hsd_from_Mmd_262_l | 3.349068609 | −1.059783835 | 4.408852444 |
| HSbrain_678_l | 2.702675258 | −1.706371763 | 4.409047021 |
| hsa-mir-142_r | 0.446226699 | −3.963478719 | 4.409705419 |
| HS_from_MQbrain_1286_l | 3.236714118 | −1.176377798 | 4.413091916 |
| HS_from_MQbrain_536_l | 1.766898397 | −2.649369834 | 4.416268231 |
| HSbrain_61_l | 1.751028569 | −2.665635932 | 4.4166645 |
| HSbrain_687_r | 1.53020899 | −2.886588647 | 4.416797637 |
| HSbrain_1_l | 2.110848824 | −2.308051191 | 4.418900016 |
| HSbrain_308_l | 3.658177289 | −0.760843683 | 4.419020972 |
| hsa-mir-139_r | 3.346487837 | −1.072811228 | 4.419299065 |
| HSbrain_550_l | 1.974672856 | −2.447238634 | 4.42191149 |
| HS_from_PT_305_l | 3.254701005 | −1.168910361 | 4.423611366 |
| Hsd_from_Mmd_468_l | 3.370104224 | −1.053626532 | 4.423730757 |
| HS_from_MM_172_l | 1.169829365 | −3.256401768 | 4.426231133 |
| HS_from_MM_283_r | 3.179469573 | −1.247560791 | 4.427030364 |
| hsa-mir-372_l | 3.260736336 | −1.167100664 | 4.427837 |
| HS_from_MQbrain_1585_r | 1.655543967 | −2.776140391 | 4.431684358 |
| HS_1_l | 2.833567324 | −1.599596683 | 4.433164007 |
| hsa-mir-23b_l | 3.011159748 | −1.422148221 | 4.433307969 |
| HS_282_l | 3.513870872 | −0.920969256 | 4.434840128 |
| HS_from_MQbrain_738_l | 2.929959668 | −1.505719088 | 4.435678756 |
| HSbrain_449_r | 1.730175496 | −2.709151333 | 4.439326829 |
| HS_from_MQbrain_1279_r | 2.440884923 | −2.000568538 | 4.441453461 |
| HS_from_MQbrain_99_l | 2.627467917 | −1.814476687 | 4.441944603 |
| HSbrain_325_r | 1.930462567 | −2.512357187 | 4.442819754 |
| HSbrain_210_r | 1.917791002 | −2.529124663 | 4.446915666 |
| HS_from_MQbrain_112_r | 2.944931331 | −1.503200661 | 4.448131992 |
| hsa-mir-769_l | 3.23305404 | −1.218437305 | 4.451491345 |
| HSbrain_667_l | 2.847266892 | −1.604802788 | 4.45206968 |
| HSbrain_683_l | 2.821294245 | −1.631661787 | 4.452956032 |
| HSbrain_45_l | 2.101526263 | −2.351898115 | 4.453424377 |
| Hsd_from_Mmd_294_l | 2.456098611 | −1.998348032 | 4.454446643 |
| HS_from_MQbrain_2689_r | 3.324416377 | −1.131968296 | 4.456384674 |
| HSbrain_427_l | 3.816463754 | −0.645996005 | 4.462459759 |
| Hsd_from_Mmd_437_r | 2.587319388 | −1.875230042 | 4.462549431 |
| HSbrain_427_r | 3.274557686 | −1.189431959 | 4.463989645 |
| hsa-mir-519c_r | 1.411687272 | −3.055395521 | 4.467082793 |
| HS_from_MM_292_l | 1.330865479 | −3.138464895 | 4.469330374 |
| HS_from_MQbrain_458_r | 2.762590489 | −1.708300044 | 4.470890533 |
| HS_from_MQbrain_270_l | 2.427057688 | −2.044857075 | 4.471914763 |
| HS_from_MQbrain_1112_r | 2.836725338 | −1.636028691 | 4.472754029 |
| HS_from_MQbrain_1546_r | 2.026857743 | −2.446318376 | 4.473176119 |
| Hsd_51_r | 3.268384618 | −1.205432003 | 4.473816621 |
| HS_from_PT_3_l | 2.862514315 | −1.611509863 | 4.474024179 |
| HS_from_PT_155_r | 3.146539722 | −1.328212909 | 4.474752632 |
| HS_from_MQbrain_1792_l | 3.694645407 | −0.780305388 | 4.474950795 |
| HS_from_MQbrain_366_r | 2.699618632 | −1.776038681 | 4.475657313 |
| HS_from_MQbrain_883_l | 3.69681464 | −0.779545386 | 4.476360026 |
| HS_from_MM_105_l | 1.711052783 | −2.767859192 | 4.478911975 |
| hsa-let-7a-2_l | 1.283354756 | −3.199923534 | 4.48327829 |
| HSbrain_356_r | 2.144223548 | −2.340345355 | 4.484568903 |
| HS_from_MQbrain_350_r | 3.208448604 | −1.27992208 | 4.488370684 |
| HS_266_r | 2.936367584 | −1.552977041 | 4.489344625 |
| HS_255_l | 3.054441067 | −1.436439891 | 4.490880957 |
| Hsd_52_l | 2.877644595 | −1.615144472 | 4.492789067 |
| HS_from_MQbrain_118_r | 3.603659469 | −0.890473388 | 4.494132857 |
| HSbrain_580_l | 1.496287112 | −2.998426375 | 4.494713487 |
| HS_270_l | 3.593561927 | −0.906035898 | 4.499597825 |
| HS_from_MQbrain_1092_l | 1.899448204 | −2.601928834 | 4.501377038 |
| Hsd_from_Mmd_226_l | 1.31223755 | −3.192258052 | 4.504495602 |
| HSbrain_387_l | 2.87116755 | −1.633510028 | 4.504677579 |
| HSbrain_449_l | 2.600768007 | −1.907137901 | 4.507905908 |
| HSbrain_137_r | 3.092871639 | −1.415834041 | 4.508705681 |
| Hsd_from_Mmd_69_r | 1.332468024 | −3.17780478 | 4.510272804 |
| HSbrain_1305_l | 3.070453573 | −1.439904175 | 4.510357748 |
| HSbrain_559_r | 1.311678919 | −3.200744976 | 4.512423895 |
| hsa-mir-522_r | 2.464862857 | −2.048022208 | 4.512885065 |
| HS_from_MQbrain_1782_r | 1.639263872 | −2.87373984 | 4.513003712 |
| HS_from_MQbrain_1507_r | 3.842852849 | −0.670182731 | 4.51303558 |
| HS_from_MQbrain_1552_l | 3.85137131 | −0.662960439 | 4.51433175 |
| HS_215_r | 3.47796408 | −1.037817097 | 4.515781178 |
| HS_from_MQbrain_471_l | 2.580477757 | −1.935713839 | 4.516191596 |
| HS_from_MQbrain_1558_l | 3.698728559 | −0.818218931 | 4.516947491 |
| HSbrain_314_r | 3.203836216 | −1.314579638 | 4.518415854 |
| HS_from_MQbrain_1620_r | 2.297687402 | −2.22075291 | 4.518440312 |
| HSbrain_248_r | 2.625010945 | −1.894332557 | 4.519343501 |
| HS_from_MM_120_l | 3.088821529 | −1.430865824 | 4.519687353 |
| hsa-mir-153-2_l | 1.040941445 | −3.481560724 | 4.522502169 |
| HS_from_MQbrain_1034_r | 3.127449244 | −1.396056107 | 4.523505351 |
| HS_from_PT_26_r | 2.941680303 | −1.583530072 | 4.525210374 |
| Hsd_from_Mmd_197_r | 2.725489619 | −1.800780811 | 4.52627043 |
| HSbrain_223_r | 3.480320951 | −1.046593962 | 4.526914914 |
| HS_from_MQbrain_709_r | 1.922231476 | −2.605796413 | 4.528027889 |
| HS_303_l | 1.884663028 | −2.649317147 | 4.533980175 |
| HS_from_MQbrain_346_r | 2.619417953 | −1.916393902 | 4.535811855 |
| HSbrain_525_r | 1.903359782 | −2.63259443 | 4.535954212 |
| HS_from_MQbrain_14_l | 3.160424873 | −1.379272393 | 4.539697267 |
| HS_from_MQbrain_72_l | 3.8663402 | −0.674416791 | 4.540756991 |
| HS_from_MQbrain_131_l | 3.577445645 | −0.964918599 | 4.542364244 |
| HS_293_r | 3.726624217 | −0.816128471 | 4.542752689 |
| HSbrain_198_r | 3.254203304 | −1.28980211 | 4.544005414 |
| Hsd_69_l | 2.893291378 | −1.650766011 | 4.54405739 |
| HS_239_r | 3.393133156 | −1.152324612 | 4.545457769 |
| HS_243_l | 2.925307082 | −1.623932342 | 4.549239424 |
| HS_from_MM_103_r | 2.703439033 | −1.845835577 | 4.549274609 |
| HSbrain_110_l | 1.645810531 | −2.903885229 | 4.54969576 |
| HS_from_MQbrain_118_l | 3.283241084 | −1.266561381 | 4.549802464 |
| HS_from_MM_283_l | 2.767294378 | −1.785355658 | 4.552650036 |
| HSbrain_170_l | 3.52711995 | −1.025868775 | 4.552988725 |
| HSbrain_608_r | 2.871169843 | −1.682701268 | 4.553871111 |
| HS_from_MQbrain_1612_r | 2.042791017 | −2.511168193 | 4.55395921 |
| HS_from_MQbrain_594_l | 3.046755258 | −1.509472285 | 4.556227542 |
| HS_from_MQbrain_484_r | 2.761435223 | −1.799275478 | 4.560710701 |
| hsa-mir-7-3_l | 2.956272869 | −1.604442698 | 4.560715567 |
| HSbrain_806_l | 2.576344432 | −1.985092208 | 4.561436641 |
| hsa-mir-644_l | 2.139841722 | −2.422250025 | 4.562091746 |
| HS_from_MM_153_r | 3.176592499 | −1.387208126 | 4.563800625 |
| HS_from_PT_40_l | 2.29564242 | −2.271794708 | 4.567437128 |
| Hsd_from_Mmd_353_r | 2.908039613 | −1.664269151 | 4.572308764 |
| HS_from_MQbrain_478_l | 3.87424015 | −0.698209618 | 4.572449768 |
| Hsd_from_Mmd_494_l | 2.062189458 | −2.511111728 | 4.573301186 |
| HS_from_MQbrain_76_r | 3.463870673 | −1.109572924 | 4.573443598 |
| HSbrain_712_r | 2.210055746 | −2.3651778 | 4.575233545 |
| hsa-mir-615_r | 2.872152528 | −1.70512199 | 4.577274517 |
| HS_from_MQbrain_325_r | 2.013062823 | −2.564421029 | 4.577483852 |
| HSbrain_136_l | 3.262845735 | −1.317027782 | 4.579873517 |
| HSbrain_1437_r | 2.641918297 | −1.938728227 | 4.580646525 |
| HS_from_MQbrain_436_r | 1.338217093 | −3.243051772 | 4.581268865 |
| HS_from_MQbrain_465_l | 3.494207424 | −1.087087336 | 4.58129476 |
| HS_38_r | 1.930212885 | −2.657422927 | 4.587635811 |
| HS_143_l | 2.688590054 | −1.899072336 | 4.58766239 |
| HS_269_l | 3.59985888 | −0.987989834 | 4.587848713 |
| hsa-mir-16-1_l | 1.713130507 | −2.877698901 | 4.590829408 |
| HSbrain_297_r | 1.857831981 | −2.737274685 | 4.595106666 |
| HS_from_MQbrain_1723_l | 3.16533212 | −1.430651344 | 4.595983464 |
| HSbrain_725_l | 2.603417138 | −1.993933701 | 4.597350839 |
| HS_291_r | 2.657557971 | −1.940041489 | 4.597599459 |
| HSbrain_27_l | 1.1841553 | −3.415107586 | 4.599262886 |
| hsa-mir-15a_l | 1.311847976 | −3.289656486 | 4.601504462 |
| HS_from_MQbrain_1325_l | 3.274854262 | −1.327730524 | 4.602584786 |
| hsa-mir-559_l | 1.74049034 | −2.863272859 | 4.603763199 |
| HS_296_r | 3.670682358 | −0.934935245 | 4.605617603 |
| HS_from_MQbrain_1463_r | 3.023733191 | −1.582584711 | 4.606317903 |
| hsa-mir-10b_l | 2.441199589 | −2.165609909 | 4.606809498 |
| HS_from_MM_40_r | 3.816348891 | −0.79143766 | 4.607786551 |
| HS_from_MQbrain_656_l | 3.320716895 | −1.287500265 | 4.608217159 |
| HS_from_MQbrain_1584_r | 1.583737691 | −3.024762003 | 4.608499694 |
| hsa-mir-221_l | 3.188155557 | −1.423138768 | 4.611294325 |
| HS_from_MQbrain_1958_l | 3.640767565 | −0.97169984 | 4.612467406 |
| HS_from_MQbrain_343_r | 3.352393225 | −1.2610524 | 4.613445626 |
| hsa-mir-184_l | 1.923843766 | −2.691092784 | 4.614936551 |
| HS_224_l | 3.493750704 | −1.123113155 | 4.61686386 |
| Hsd_49_l | 1.631949419 | −2.985919283 | 4.617868703 |
| HS_from_MM_305_r | 1.966746456 | −2.651920635 | 4.618667091 |
| Hsd_from_Mmd_412_r | 1.409505326 | −3.20932782 | 4.618833146 |
| HSbrain_203_r | 3.519377561 | −1.099634942 | 4.619012503 |
| hsa-mir-7-2_l | 3.014182935 | −1.606124546 | 4.620307481 |
| HSbrain_116_r | 1.382934557 | −3.24282889 | 4.625763448 |
| HS_from_MQbrain_151_l | 3.212830892 | −1.413130286 | 4.625961178 |
| hsa-mir-129-1_r | 3.465041908 | −1.161114235 | 4.626156143 |
| hsa-mir-127_l | 4.034286121 | −0.593971364 | 4.628257485 |
| HS_from_MQbrain_211_l | 2.75908417 | −1.873385059 | 4.632469229 |
| HSbrain_170_r | 2.472068757 | −2.162096701 | 4.634165457 |
| HSbrain_178_r | 3.482942501 | −1.151768997 | 4.634711497 |
| HS_from_MM_155_l | 1.38167318 | −3.253679639 | 4.635352819 |
| HSbrain_389_l | 3.225268717 | −1.41225515 | 4.637523867 |
| HS_from_MQbrain_1503_r | 3.942616983 | −0.697998271 | 4.640615253 |
| Hsd_from_Mmd_461_l | 4.389062425 | −0.252921143 | 4.641983569 |
| HSbrain_454_r | 3.63277761 | −1.010458004 | 4.643235614 |
| HS_from_MQbrain_1795_r | 3.225071288 | −1.420381056 | 4.645452344 |
| HS_from_PT_206_r | 1.224782289 | −3.420691168 | 4.645473457 |
| HS_from_MQbrain_1439_l | 3.860235729 | −0.789688181 | 4.64992391 |
| HSbrain_284_l | 1.934599805 | −2.718177339 | 4.652777144 |
| HS_193_l | 2.258310122 | −2.394683737 | 4.652993859 |
| hsa-let-7a-1_l | 1.260886496 | −3.393741485 | 4.654627981 |
| HSbrain_505_r | 4.222171738 | −0.433219031 | 4.655390769 |
| HS_from_PT_251_l | 3.538833617 | −1.116582673 | 4.655416291 |
| HS_from_MM_20_r | 2.703859402 | −1.951881337 | 4.655740739 |
| hsa-mir-203_r | 3.294573295 | −1.361989825 | 4.65656312 |
| HSbrain_697_l | 3.814563494 | −0.842836292 | 4.657399785 |
| HS_from_MQbrain_1323_l | 3.526017505 | −1.132277159 | 4.658294664 |
| HS_from_MM_163_l | 1.614626536 | −3.044691758 | 4.659318293 |
| HS_from_MQbrain_1378_l | 4.142050526 | −0.517476259 | 4.659526785 |
| Hsd_60_l | 4.155701156 | −0.503883924 | 4.65958508 |
| hsa-mir-421_r | 2.871695273 | −1.788332271 | 4.660027544 |
| HSbrain_892_l | 1.838090774 | −2.823450887 | 4.661541661 |
| HS_from_MQbrain_1087_r | 3.6055718 | −1.057505619 | 4.663077419 |
| HS_from_MQbrain_825_l | 2.849639526 | −1.816395137 | 4.666034664 |
| HSbrain_672_l | 2.852249215 | −1.814168504 | 4.666417719 |
| HS_from_MQbrain_1145_r | 2.155166385 | −2.511643096 | 4.666809481 |
| HS_295_l | 3.611931466 | −1.056759345 | 4.668690811 |
| HS_from_MQbrain_915_r | 3.122038749 | −1.550797092 | 4.672835841 |
| hsa-mir-381_r | 4.065989059 | −0.607318147 | 4.673307206 |
| HS_from_MM_191_r | 1.85244859 | −2.821037944 | 4.673486534 |
| HS_from_PT_218_l | 1.615608436 | −3.058489618 | 4.674098054 |
| HS_from_MQbrain_801_r | 3.017891127 | −1.65979213 | 4.677683257 |
| HS_from_MQbrain_16_l | 4.599967046 | −0.078619751 | 4.678586798 |
| hsa-mir-650_l | 2.937629839 | −1.745153308 | 4.682783147 |
| hsa-mir-192_l | 2.527501562 | −2.15582679 | 4.683328352 |
| Hsd_from_Mmd_376_l | 3.879796479 | −0.803578775 | 4.683375254 |
| Hsd_from_Mmd_328_l | 3.472263136 | −1.212889518 | 4.685152653 |
| HS_from_MQbrain_1290_r | 3.510149512 | −1.176216936 | 4.686366448 |
| HSbrain_542_r | 4.274225607 | −0.412195881 | 4.686421489 |
| HS_from_MQbrain_284_r | 3.667030032 | −1.02136734 | 4.688397372 |
| HSbrain_821_l | 1.84549341 | −2.843343708 | 4.688837118 |
| HSbrain_219_l | 3.232284116 | −1.457514192 | 4.689798308 |
| hsa-mir-210_r | 3.775218629 | −0.914635749 | 4.689854378 |
| HSbrain_1286_l | 3.83059884 | −0.860230199 | 4.690829039 |
| HSbrain_1386_l | 2.98072241 | −1.711947185 | 4.692669595 |
| HS_from_MQbrain_1514_l | 3.704802336 | −0.989467433 | 4.694269769 |
| HS_294_l | 3.739276109 | −0.956345183 | 4.695621293 |
| HS_from_MQbrain_787_l | 3.540784146 | −1.1561351 | 4.696919246 |
| HS_230_l | 3.579499332 | −1.119718794 | 4.699218125 |
| HS_from_MQbrain_1180_r | 3.461732885 | −1.240753943 | 4.702486828 |
| HS_from_MQbrain_114_l | 1.642277041 | −3.060349557 | 4.702626598 |
| HS_245_r | 2.530846625 | −2.172124697 | 4.702971322 |
| HSbrain_624_r | 1.722472157 | −2.980985395 | 4.703457552 |
| HS_from_MM_187_r | 3.765531037 | −0.938377757 | 4.703908794 |
| hsa-mir-196a-1_r | 2.744883754 | −1.961319163 | 4.706202917 |
| HS_227_l | 3.55500649 | −1.152895607 | 4.707902097 |
| HS_from_MQbrain_787_r | 1.558863027 | −3.149529499 | 4.708392526 |
| hsa-mir-181d_r | 2.987972963 | −1.723753302 | 4.711726264 |
| HS_94_l | 1.969693174 | −2.745533312 | 4.715226487 |
| hsa-mir-302b_r | 1.691313911 | −3.025743438 | 4.717057348 |
| HSbrain_393_r | 2.74572076 | −1.97184178 | 4.717562541 |
| hsa-mir-552_l | 1.641371363 | −3.077124298 | 4.718495662 |
| HS_from_MQbrain_701_l | 1.860679784 | −2.858977215 | 4.719656999 |
| HS_from_PT_121_l | 3.514910587 | −1.205511603 | 4.72042219 |
| HSbrain_685_r | 2.94857295 | −1.777872924 | 4.726445874 |
| hsa-mir-602_r | 3.540750869 | −1.186860327 | 4.727611196 |
| HS_from_PT_113_r | 3.13333352 | −1.595510169 | 4.728843689 |
| hsa-mir-24-1_r | 2.798304346 | −1.93259045 | 4.730894796 |
| Hsd_from_Mmd_40_l | 4.029351576 | −0.704252338 | 4.733603914 |
| HS_from_PT_203_r | 1.613246274 | −3.124432272 | 4.737678546 |
| HSbrain_279_l | 3.384031798 | −1.353831919 | 4.737863717 |
| HS_from_PT_194_l | 0.924714762 | −3.813857839 | 4.738572601 |
| HSbrain_304_l | 3.070625303 | −1.671021556 | 4.741646859 |
| HSbrain_270_l | 3.051575508 | −1.69024559 | 4.741821098 |
| HS_187_r | 4.181054383 | −0.563707635 | 4.744762018 |
| HS_from_MQbrain_1734_l | 4.25369456 | −0.491487493 | 4.745182053 |
| HS_from_MQbrain_1343_r | 2.106607573 | −2.641191028 | 4.747798601 |
| HS_from_MQbrain_353_r | 3.224065244 | −1.524931275 | 4.748996519 |
| HS_from_MQbrain_1238_r | 3.797414496 | −0.95218608 | 4.749600576 |
| hsa-let-7a-3_l | 1.313035955 | −3.439225691 | 4.752261645 |
| HSbrain_121_r | 3.274067942 | −1.47858121 | 4.752649152 |
| Hsd_from_Mmd_228_l | 1.364401048 | −3.391566948 | 4.755967996 |
| HS_from_MQbrain_1810_l | 2.464713896 | −2.291639748 | 4.756353645 |
| hsa-mir-22_r | 2.327475186 | −2.431711551 | 4.759186737 |
| HS_from_MQbrain_931_r | 3.337852904 | −1.42441839 | 4.762271294 |
| Hsd_36_l | 1.792951533 | −2.96936417 | 4.762315703 |
| HSbrain_398_l | 2.684490701 | −2.078898807 | 4.763389509 |
| HS_from_MQbrain_1551_l | 3.884000425 | −0.880841465 | 4.76484189 |
| HS_from_MQbrain_315_l | 3.782987997 | −0.985110598 | 4.768098595 |
| HSbrain_655_r | 3.738631397 | −1.031243068 | 4.769874465 |
| Hsd_from_Mmd_276_l | 4.297154523 | −0.475301178 | 4.772455702 |
| HS_from_MQbrain_792_l | 2.864158787 | −1.911891394 | 4.776050181 |
| Hsd_54_l | 3.286974174 | −1.491907307 | 4.778881481 |
| hsa-mir-193b_l | 2.727217972 | −2.056913463 | 4.784131435 |
| hsa-mir-614_l | 3.463898896 | −1.322148739 | 4.786047634 |
| HS_from_MQbrain_476_l | 3.995342346 | −0.791737922 | 4.787080269 |
| hsa-mir-500_r | 3.658269534 | −1.128950477 | 4.787220011 |
| HS_57_r | 3.895038619 | −0.89282786 | 4.787866479 |
| HSbrain_771_r | 1.712937445 | −3.075072903 | 4.788010348 |
| HS_from_MQbrain_1728_r | 3.313868672 | −1.477206863 | 4.791075535 |
| HS_from_MQbrain_234_l | 4.453471636 | −0.338793312 | 4.792264948 |
| HSbrain_486_l | 3.965014616 | −0.829489353 | 4.794503969 |
| hsa-mir-16-2_l | 2.037765015 | −2.758194516 | 4.795959531 |
| HS_from_MQbrain_1048_l | 4.009645727 | −0.787303704 | 4.79694943 |
| HS_from_MQbrain_637_l | 3.498843713 | −1.299597114 | 4.798440828 |
| HS_from_MQbrain_297_r | 3.448132327 | −1.354086506 | 4.802218833 |
| HS_from_PT_44_r | 3.263050978 | −1.539848565 | 4.802899542 |
| HS_from_MM_13_r | 3.263480901 | −1.540007607 | 4.803488508 |
| hsa-mir-768_r | 2.589938983 | −2.216918992 | 4.806857975 |
| HS_from_MQbrain_960_l | 2.606113508 | −2.203471342 | 4.80958485 |
| HS_31_l | 3.22456319 | −1.585254746 | 4.809817936 |
| HS_from_MM_96_l | 2.855031474 | −1.954884854 | 4.809916329 |
| HS_from_PT_7_l | 3.346007287 | −1.464276638 | 4.810283925 |
| HS_from_PT_258_r | 2.241699745 | −2.569827569 | 4.811527314 |
| Hsd_from_Mmd_274_l | 3.091015535 | −1.722379515 | 4.81339505 |
| HS_from_MQbrain_751_l | 3.347608177 | −1.466031951 | 4.813640127 |
| HS_from_MQbrain_31_r | 4.073436603 | −0.740794212 | 4.814230815 |
| HSbrain_333_r | 3.313933938 | −1.50150797 | 4.815441908 |
| HSbrain_278_l | 3.014590423 | −1.802503646 | 4.817094069 |
| HSbrain_536_l | 3.392835933 | −1.427196124 | 4.820032057 |
| HSbrain_342_l | 2.719428812 | −2.102932954 | 4.822361766 |
| HSbrain_496_l | 3.009231766 | −1.818080955 | 4.82731272 |
| HSbrain_362_r | 2.710368961 | −2.121158851 | 4.831527812 |
| HS_234_l | 3.673093797 | −1.158581679 | 4.831675476 |
| HS_from_MM_290_l | 1.931507501 | −2.904133218 | 4.83564072 |
| hsa-mir-135a-2_l | 1.443761332 | −3.393789375 | 4.837550707 |
| HS_from_MQbrain_1246_r | 3.167779633 | −1.670405213 | 4.838184845 |
| HS_271_r | 3.688454263 | −1.150107795 | 4.838562058 |
| hsa-mir-195_l | 1.315266951 | −3.526131828 | 4.841398779 |
| HSbrain_218_l | 1.933765045 | −2.909864581 | 4.843629626 |
| HS_from_MQbrain_870_r | 3.780725549 | −1.065328564 | 4.846054112 |
| HS_from_MQbrain_1901_l | 1.567447504 | −3.279664791 | 4.847112294 |
| Hsd_87_l | 2.715380529 | −2.133080693 | 4.848461222 |
| HSbrain_202_r | 2.894163056 | −1.956817817 | 4.850980873 |
| HSbrain_288_r | 3.627702358 | −1.224190854 | 4.851893213 |
| hsa-mir-221_r | 2.243776712 | −2.60842676 | 4.852203472 |
| Hsd_83_r | 2.312030239 | −2.540245962 | 4.8522762 |
| HSbrain_638_r | 4.263737826 | −0.591230463 | 4.854968289 |
| HSbrain_156_r | 3.832628684 | −1.025783562 | 4.858412246 |
| Hsd_from_Mmd_190_l | 2.96540259 | −1.894736437 | 4.860139027 |
| hsa-mir-7-1_l | 3.254137311 | −1.607608592 | 4.861745902 |
| HSbrain_603_l | 3.378028895 | −1.484260577 | 4.862289472 |
| hsa-mir-498_l | 2.857356323 | −2.006366908 | 4.863723231 |
| HS_298_l | 3.245727352 | −1.618515418 | 4.86424277 |
| HS_from_MQbrain_1082_l | 3.038041153 | −1.828692848 | 4.866734001 |
| HS_from_MQbrain_1212_r | 1.726440526 | −3.140455926 | 4.866896452 |
| HSbrain_539_r | 4.254297153 | −0.61404578 | 4.868342934 |
| HS_from_MQbrain_319_l | 1.271264614 | −3.599037252 | 4.870301866 |
| HS_270_r | 3.807800171 | −1.063485635 | 4.871285805 |
| HSbrain_83_l | 4.757960986 | −0.113347793 | 4.871308779 |
| HS_242_r | 2.943246653 | −1.929069135 | 4.872315788 |
| HS_238_l | 3.778030931 | −1.096875033 | 4.874905964 |
| HS_from_MQbrain_399_l | 2.300484061 | −2.575422631 | 4.875906693 |
| HS_236_l | 3.868926399 | −1.007140666 | 4.876067065 |
| hsa-mir-150_r | 4.007829804 | −0.868328259 | 4.876158063 |
| HS_from_MM_82_r | 4.04313532 | −0.83522739 | 4.878362709 |
| HS_from_MM_359_r | 2.887847105 | −1.991716996 | 4.879564102 |
| HSbrain_631_r | 2.878955855 | −2.001762682 | 4.880718537 |
| Hsd_from_Mmd_26_r | 1.582237121 | −3.299428257 | 4.881665378 |
| hsa-let-7b_r | 2.255968183 | −2.62716493 | 4.883133114 |
| HS_from_MQbrain_441_l | 3.163026535 | −1.72251182 | 4.885538355 |
| HS_from_MQbrain_2700_r | 2.72330886 | −2.162562338 | 4.885871198 |
| Hsd_from_Mmd_80_l | 1.626004464 | −3.260603521 | 4.886607985 |
| HSbrain_650_r | 4.051139114 | −0.837554055 | 4.888693169 |
| HS_from_MM_192_l | 1.500793799 | −3.390697473 | 4.891491271 |
| HS_293_l | 3.736043938 | −1.156903247 | 4.892947185 |
| hsa-mir-611_r | 4.127661712 | −0.765879211 | 4.893540923 |
| hsa-mir-18b_l | 3.366454205 | −1.531598877 | 4.898053082 |
| HS_from_MQbrain_1180_l | 2.86662064 | −2.031634669 | 4.898255309 |
| hsa-mir-99b_l | 3.220612794 | −1.677890004 | 4.898502798 |
| HSbrain_718_r | 1.879461783 | −3.019233792 | 4.898695575 |
| hsa-mir-517a_r | 3.323648272 | −1.575175801 | 4.898824073 |
| HS_from_MQbrain_238_r | 3.888716774 | −1.010966178 | 4.899682951 |
| HS_from_MQbrain_593_l | 4.095851317 | −0.80414063 | 4.899991947 |
| HS_from_MQbrain_694_l | 3.545397631 | −1.360080526 | 4.905478156 |
| hsa-mir-503_l | 3.294641284 | −1.611368203 | 4.906009487 |
| HSbrain_395_r | 3.211007963 | −1.697550148 | 4.90855811 |
| HS_from_MQbrain_215_l | 3.06264762 | −1.847586802 | 4.910234422 |
| HS_from_MQbrain_1636_l | 3.627089647 | −1.283917526 | 4.911007173 |
| HS_from_MQbrain_478_r | 3.361299201 | −1.550505901 | 4.911805101 |
| HS_from_MQbrain_659_l | 3.747111102 | −1.165383071 | 4.912494173 |
| HS_from_MQbrain_1672_l | 2.154788884 | −2.757967795 | 4.912756679 |
| HSbrain_733_l | 2.368226164 | −2.544612556 | 4.912838719 |
| HS_from_MQbrain_954_l | 2.257530912 | −2.658631141 | 4.916162054 |
| HS_from_MQbrain_402_r | 2.522419295 | −2.396131891 | 4.918551186 |
| HS_from_MQbrain_1334_r | 1.60360394 | −3.316561509 | 4.92016545 |
| hsa-mir-100_l | 1.936797577 | −2.986252743 | 4.923050321 |
| HSbrain_567_r | 4.168866772 | −0.755661684 | 4.924528456 |
| HSbrain_1390_l | 2.883536151 | −2.04232685 | 4.925863001 |
| HS_from_MQbrain_1353_l | 1.831945649 | −3.097732139 | 4.929677789 |
| HS_297_l | 3.658138951 | −1.272758506 | 4.930897457 |
| hsa-mir-451_l | 2.860114507 | −2.079355672 | 4.93947018 |
| HS_from_MQbrain_687_l | 3.520203571 | −1.422585034 | 4.942788605 |
| HS_from_MQbrain_920_l | 3.643544221 | −1.300173672 | 4.943717893 |
| HS_from_MQbrain_864_l | 3.495420383 | −1.44897999 | 4.944400374 |
| HS_from_MQbrain_708_r | 3.760727675 | −1.18697574 | 4.947703415 |
| HSbrain_1_r | 3.779079405 | −1.169504771 | 4.948584176 |
| HS_from_MM_40_l | 4.541405432 | −0.410590356 | 4.951995787 |
| HS_from_MQbrain_693_l | 3.671566694 | −1.28293922 | 4.954505913 |
| HS_from_MQbrain_295_l | 3.542603651 | −1.415423739 | 4.958027391 |
| HS_from_MQbrain_349_r | 3.40314322 | −1.562680299 | 4.96582352 |
| hsa-mir-7-2_r | 2.308679799 | −2.657442201 | 4.966122 |
| HS_from_MQbrain_1551_r | 3.278055161 | −1.692740815 | 4.970795976 |
| HS_44_r | 1.672788352 | −3.298341886 | 4.971130238 |
| HS_from_MQbrain_1671_r | 3.387447413 | −1.583907189 | 4.971354602 |
| HS_from_PT_303_l | 3.609655813 | −1.364530153 | 4.974185966 |
| HS_226_l | 3.413696096 | −1.560559144 | 4.97425524 |
| HSbrain_149_l | 3.40164818 | −1.574699619 | 4.976347799 |
| HS_192_l | 3.463427645 | −1.513330625 | 4.976758269 |
| hsa-mir-10a_l | 2.764589881 | −2.215232301 | 4.979822182 |
| HS_from_MQbrain_110_r | 4.47883335 | −0.520780129 | 4.999613479 |
| HS_from_MQbrain_1832_r | 1.664160694 | −3.337085827 | 5.001246521 |
| HS_from_MQbrain_1209_r | 4.121755836 | −0.879515257 | 5.001271093 |
| HS_28_r | 2.506035722 | −2.497749025 | 5.003784746 |
| Hsd_from_Mmd_149_l | 3.042130919 | −1.962241796 | 5.004372716 |
| HS_147_l | 3.847931471 | −1.162487129 | 5.0104186 |
| Hsd_50_r | 3.891133142 | −1.123087161 | 5.014220303 |
| HSbrain_321_l | 4.222442609 | −0.793746716 | 5.016189325 |
| Hsd_from_Mmd_91_l | 1.75725669 | −3.25953709 | 5.016793781 |
| HSbrain_422_r | 4.616518979 | −0.409413813 | 5.025932792 |
| HS_from_MQbrain_374_r | 3.088425979 | −1.945101148 | 5.033527127 |
| HS_296_l | 3.691753934 | −1.342843015 | 5.03459695 |
| Hsd_from_Mmd_343_l | 3.153669719 | −1.881983743 | 5.035653463 |
| HSbrain_553_r | 3.023799224 | −2.014682079 | 5.038481303 |
| HS_from_MQbrain_240_r | 3.559428342 | −1.486398141 | 5.045826483 |
| HS_from_MQbrain_1318_r | 3.842538771 | −1.203331739 | 5.04587051 |
| HS_221_l | 3.666584393 | −1.382013375 | 5.048597768 |
| HSbrain_57_l | 4.384467535 | −0.664284292 | 5.048751827 |
| hsa-mir-565_r | 2.606965747 | −2.44387871 | 5.050844458 |
| HSbrain_239_l | 3.543168484 | −1.514224544 | 5.057393029 |
| Hsd_from_Mmd_411_l | 4.050389674 | −1.009209988 | 5.059599662 |
| HS_174_l | 2.798895904 | −2.263723875 | 5.062619779 |
| HS_from_MQbrain_1147_r | 2.005031358 | −3.057860126 | 5.062891484 |
| HS_from_MQbrain_575_r | 2.969253009 | −2.094812156 | 5.064065166 |
| HS_from_MQbrain_973_r | 3.283047874 | −1.781131015 | 5.064178888 |
| HSbrain_426_l | 1.956473761 | −3.108397502 | 5.064871263 |
| HS_from_MQbrain_1863_l | 0.639055626 | −4.426586344 | 5.06564197 |
| HS_from_MQbrain_1552_r | 3.420250449 | −1.646229571 | 5.06648002 |
| HSbrain_429_l | 3.774359349 | −1.297650721 | 5.07201007 |
| hsa-mir-135a-1_l | 1.499107172 | −3.573907366 | 5.073014539 |
| hsa-mir-181a-1_l | 1.793713318 | −3.283917125 | 5.077630443 |
| HS_from_MQbrain_1175_l | 4.296965999 | −0.782331004 | 5.079297003 |
| HS_from_MM_145_l | 4.10808325 | −0.972938521 | 5.081021771 |
| hsa-mir-181a-2_l | 1.588474029 | −3.494881079 | 5.083355108 |
| HS_from_MQbrain_599_r | 3.257923847 | −1.827189852 | 5.085113699 |
| HS_from_MQbrain_192_l | 3.426958595 | −1.65937693 | 5.086335524 |
| HSbrain_563_r | 2.031249955 | −3.056601747 | 5.087851701 |
| HS_from_MQbrain_1560_l | 2.467180549 | −2.625032361 | 5.09221291 |
| Hsd_from_Mmd_284_r | 2.655380318 | −2.437192118 | 5.092572436 |
| HS_205_l | 4.497520544 | −0.595149237 | 5.092669781 |
| HSbrain_789_r | 4.560892088 | −0.533678481 | 5.094570569 |
| HS_from_MQbrain_1606_r | 3.757884262 | −1.338282135 | 5.096166397 |
| HSbrain_159_r | 4.915227951 | −0.1863132 | 5.101541151 |
| HS_from_PT_195_l | 4.0936569 | −1.008393271 | 5.102050171 |
| hsa-mir-602_l | 4.073233665 | −1.034079832 | 5.107313496 |
| hsa-mir-24-2_r | 3.043402392 | −2.065397837 | 5.108800229 |
| HS_93_l | 4.032506399 | −1.078035098 | 5.110541497 |
| HS_from_MQbrain_986_r | 2.686570455 | −2.424099698 | 5.110670153 |
| hsa-mir-801_l | 4.38259069 | −0.730479368 | 5.113070057 |
| HS_from_MQbrain_771_r | 2.40206001 | −2.711446095 | 5.113506104 |
| HS_from_MQbrain_1432_l | 3.27618405 | −1.839114824 | 5.115298873 |
| hsa-mir-326_l | 2.940648196 | −2.182634073 | 5.123282268 |
| HS_65_r | 1.996145069 | −3.130828915 | 5.126973984 |
| HS_180_l | 2.983951957 | −2.144700078 | 5.128652035 |
| hsa-mir-18b_r | 1.979463641 | −3.151317755 | 5.130781396 |
| HS_43_r | 3.478491086 | −1.652974563 | 5.131465649 |
| hsa-mir-27b_r | 1.106946065 | −4.025174898 | 5.132120964 |
| hsa-mir-373_l | 3.400782096 | −1.733979786 | 5.134761882 |
| HS_from_MQbrain_778_l | 3.849723243 | −1.290768764 | 5.140492008 |
| HS_from_MQbrain_708_l | 4.727897848 | −0.412642135 | 5.140539984 |
| Hsd_from_Mmd_187_r | 2.417885525 | −2.726246624 | 5.144132149 |
| HS_from_MQbrain_1601_r | 3.518530966 | −1.627291134 | 5.1458221 |
| HSbrain_805_l | 2.8792199 | −2.268912216 | 5.148132116 |
| HSbrain_223_l | 3.773237758 | −1.37527471 | 5.148512468 |
| HS_from_MQbrain_241_l | 4.409019063 | −0.740818822 | 5.149837885 |
| HS_from_MQbrain_516_l | 3.731961903 | −1.421300713 | 5.153262616 |
| HS_103_r | 3.181156033 | −1.974743918 | 5.155899951 |
| Hsd_from_Mmd_529_r | 2.622526108 | −2.537313848 | 5.159839956 |
| hsa-mir-380_l | 4.598642438 | −0.561416269 | 5.160058708 |
| HS_from_MM_93_l | 3.552677807 | −1.610726515 | 5.163404322 |
| HS_271_l | 3.690765734 | −1.477208823 | 5.167974557 |
| HS_from_MQbrain_1010_r | 3.808974254 | −1.36161643 | 5.170590685 |
| HS_from_MQbrain_640_r | 4.128384546 | −1.044972488 | 5.173357034 |
| HS_from_MQbrain_713_l | 3.970101877 | −1.205755763 | 5.17585764 |
| Hsd_from_Mmd_45_l | 4.255208484 | −0.923202105 | 5.178410589 |
| HS_from_MQbrain_687_r | 4.929705256 | −0.250929378 | 5.180634634 |
| HS_from_MQbrain_272_r | 3.991791748 | −1.19109437 | 5.182886118 |
| HSbrain_64_l | 3.947334812 | −1.238110975 | 5.185445787 |
| HS_from_MQbrain_557_l | 3.54559829 | −1.640039969 | 5.185638259 |
| HS_from_MQbrain_52_l | 3.78705098 | −1.402264784 | 5.189315765 |
| HS_from_MM_210_r | 2.484073079 | −2.707049953 | 5.191123032 |
| HS_from_MQbrain_497_r | 4.104385799 | −1.089926272 | 5.194312071 |
| HS_from_PT_216_r | 4.251217878 | −0.947559321 | 5.198777199 |
| HS_228_l | 3.733842855 | −1.465213159 | 5.199056015 |
| HSbrain_1404_l | 4.666209621 | −0.535452732 | 5.201662353 |
| HS_from_MM_268_l | 2.974107426 | −2.228999851 | 5.203107277 |
| HS_from_MQbrain_874_r | 2.161736137 | −3.043846435 | 5.205582573 |
| HSbrain_285_l | 4.114220627 | −1.094451712 | 5.208672338 |
| HS_from_PT_35_r | 4.183001303 | −1.026084249 | 5.209085553 |
| HS_from_MQbrain_827_l | 4.109900073 | −1.103162754 | 5.213062827 |
| hsa-mir-615_l | 3.74521244 | −1.469688886 | 5.214901325 |
| hsa-mir-519a-1_r | 2.816289364 | −2.399292953 | 5.215582317 |
| HS_253_r | 4.206055431 | −1.011509985 | 5.217565415 |
| HS_from_MQbrain_535_r | 3.322904452 | −1.895704159 | 5.218608611 |
| HS_from_MQbrain_1625_l | 3.862343093 | −1.357111315 | 5.219454408 |
| HS_from_MQbrain_827_r | 3.304620914 | −1.914980744 | 5.219601658 |
| HS_from_MQbrain_1820_r | 4.193120277 | −1.031492467 | 5.224612743 |
| HSbrain_11_r | 4.423014918 | −0.802072 | 5.225086918 |
| HS_from_MQbrain_280_l | 3.312828284 | −1.922504255 | 5.235332539 |
| HS_from_MQbrain_734_r | 4.261730669 | −0.977035212 | 5.238765882 |
| HSbrain_281_r | 3.433530801 | −1.806335569 | 5.23986637 |
| HS_from_MQbrain_666_r | 3.203599212 | −2.040637249 | 5.244236461 |
| hsa-let-7i_l | 1.79628002 | −3.448940058 | 5.245220079 |
| HS_from_MQbrain_1082_l | 1.671121361 | −3.580483686 | 5.251605047 |
| HSbrain_1168_l | 3.438385288 | −1.814179309 | 5.252564597 |
| HS_203_l | 2.853775347 | −2.398893509 | 5.252668856 |
| HS_from_MQbrain_1020_r | 4.230939895 | −1.023262488 | 5.254202383 |
| HS_from_MQbrain_298_l | 1.991021091 | −3.266047475 | 5.257068566 |
| HS_from_MQbrain_1689_l | 3.098496199 | −2.16035018 | 5.258846379 |
| hsa-mir-194-2_l | 4.512111515 | −0.747268618 | 5.259380133 |
| HS_from_PT_156_l | 3.47565461 | −1.7855137 | 5.261168311 |
| HS_from_MQbrain_797_l | 3.86534469 | −1.397104049 | 5.26244874 |
| HSbrain_406_r | 4.454702816 | −0.81003235 | 5.264735165 |
| HS_from_MQbrain_238_l | 3.087539957 | −2.178610623 | 5.266150579 |
| HS_from_MQbrain_811_r | 3.066374781 | −2.202835994 | 5.269210776 |
| HS_250_r | 4.062466414 | −1.207232039 | 5.269698453 |
| HS_222_l | 3.700771895 | −1.571023484 | 5.271795379 |
| HSbrain_418_l | 4.561662378 | −0.713760039 | 5.275422417 |
| HSbrain_186_l | 3.995222297 | −1.280916841 | 5.276139138 |
| Hsd_from_Mmd_343_r | 3.74915385 | −1.528358566 | 5.277512415 |
| HSbrain_164_l | 4.018503935 | −1.261726538 | 5.280230473 |
| HS_from_PT_66_l | 3.637998024 | −1.64939961 | 5.287397634 |
| HS_from_MQbrain_816_l | 4.848657421 | −0.43894031 | 5.287597731 |
| hsa-mir-542_l | 3.341049921 | −1.947022915 | 5.288072837 |
| HS_from_MQbrain_542_l | 3.709550896 | −1.579371536 | 5.288922431 |
| HSbrain_445_r | 4.35680089 | −0.932516703 | 5.289317593 |
| HS_from_MQbrain_628_l | 3.529917584 | −1.760032633 | 5.289950216 |
| HS_from_MM_176_r | 2.733425405 | −2.557008216 | 5.290433622 |
| HS_272_l | 3.786459783 | −1.513513457 | 5.29997324 |
| HS_from_MQbrain_1493_l | 3.831014826 | −1.469908707 | 5.300923533 |
| HSbrain_379_r | 3.23587819 | −2.071753506 | 5.307631696 |
| hsa-mir-93_l | 1.882604466 | −3.426522329 | 5.309126795 |
| HS_from_MQbrain_645_l | 2.498506817 | −2.812854101 | 5.311360919 |
| HS_from_PT_301_l | 3.917612364 | −1.39468483 | 5.312297194 |
| HS_from_MQbrain_1381_l | 4.379678458 | −0.93461924 | 5.314297697 |
| HSbrain_582_l | 1.432690413 | −3.886262822 | 5.318953235 |
| HSbrain_724_l | 1.988490494 | −3.331175949 | 5.319666443 |
| HS_from_MQbrain_829_l | 4.50954359 | −0.819501435 | 5.329045025 |
| HS_from_MM_304_l | 4.325762714 | −1.004234861 | 5.329997575 |
| hsa-mir-574_r | 2.85838056 | −2.473509603 | 5.331890163 |
| HS_from_MQbrain_1550_r | 4.04701921 | −1.286517289 | 5.333536499 |
| HS_188_r | 2.600205622 | −2.733838356 | 5.334043977 |
| HS_from_MQbrain_314_r | 4.789345003 | −0.549127536 | 5.338472538 |
| HS_36_r | 4.612352422 | −0.728545264 | 5.340897687 |
| hsa-let-7f-1_l | 1.588019082 | −3.763964322 | 5.351983404 |
| Hsd_from_Mmd_291_l | 4.361840207 | −0.992699443 | 5.35453965 |
| HS_from_MQbrain_1558_r | 4.035543346 | −1.323253464 | 5.358796811 |
| HS_91_r | 3.819488244 | −1.542548961 | 5.362037205 |
| HS_2_l | 3.458196989 | −1.903902242 | 5.362099231 |
| HSbrain_368_r | 4.433887145 | −0.929879117 | 5.363766263 |
| HSbrain_616_r | 3.002236638 | −2.362695746 | 5.364932384 |
| HSbrain_100_l | 3.570227349 | −1.79471515 | 5.3649425 |
| HS_from_MQbrain_1215_r | 4.925531911 | −0.450970567 | 5.376502478 |
| HS_from_MQbrain_1879_r | 4.228976536 | −1.1529606 | 5.381937136 |
| HS_284_l | 2.666555812 | −2.71628398 | 5.382839792 |
| HSbrain_68_l | 3.874118734 | −1.511493305 | 5.385612039 |
| HS_from_PT_193_l | 4.186127732 | −1.204004669 | 5.390132401 |
| HSbrain_558_r | 4.119685364 | −1.272218165 | 5.391903529 |
| HSbrain_81_l | 4.673459962 | −0.721409572 | 5.394869535 |
| HS_233_l | 3.742243209 | −1.656301561 | 5.39854477 |
| HS_from_MQbrain_296_r | 3.304003275 | −2.095052299 | 5.399055573 |
| HSbrain_163_r | 4.382358889 | −1.022213096 | 5.404571986 |
| HSbrain_691_l | 3.473723841 | −1.94134288 | 5.415066721 |
| HS_from_PT_332_l | 3.767926637 | −1.650173792 | 5.41810043 |
| HSbrain_397_r | 3.218220646 | −2.200206295 | 5.418426941 |
| hsa-mir-17_r | 4.940072682 | −0.485631302 | 5.425703984 |
| hsa-mir-519a-2_r | 2.97948479 | −2.449215663 | 5.428700453 |
| HSbrain_222_l | 3.403657332 | −2.029974787 | 5.433632119 |
| HS_from_MM_66_r | 4.371687398 | −1.063556189 | 5.435243587 |
| HS_from_MM_66_l | 4.438051051 | −0.998643315 | 5.436694367 |
| HS_from_MQbrain_1238_l | 3.98451293 | −1.453005517 | 5.437518447 |
| HS_from_MQbrain_1638_l | 3.418178988 | −2.02094608 | 5.439125068 |
| Hsd_76_l | 3.490670938 | −1.951881927 | 5.442552865 |
| HS_from_MQbrain_545_r | 3.955126899 | −1.490012408 | 5.445139307 |
| Hsd_from_Mmd_411_r | 4.332224304 | −1.116578962 | 5.448803266 |
| hsa-mir-92b_r | 2.748668498 | −2.702394176 | 5.451062674 |
| HS_from_MQbrain_564_r | 3.294902232 | −2.156804516 | 5.451706748 |
| HSbrain_628_l | 3.082499121 | −2.376106514 | 5.458605635 |
| HSbrain_656_r | 2.982458237 | −2.477502715 | 5.459960952 |
| Hsd_14_l | 3.689163499 | −1.771700139 | 5.460863639 |
| HS_from_MQbrain_1454_r | 4.577495936 | −0.887402432 | 5.464898368 |
| HS_from_MQbrain_855_r | 3.825345577 | −1.640085564 | 5.465431141 |
| HS_from_MQbrain_599_l | 3.412798886 | −2.055944156 | 5.468743042 |
| Hsd_from_Mmd_226_r | 1.291227061 | −4.183190395 | 5.474417456 |
| HS_from_MQbrain_123_r | 3.479302649 | −1.995817976 | 5.475120624 |
| HS_301_l | 4.627077308 | −0.850607121 | 5.47768443 |
| HS_from_PT_139_l | 4.073422524 | −1.409405185 | 5.482827709 |
| Hsd_109_l | 3.72673689 | −1.759885872 | 5.486622762 |
| hsa-mir-330_r | 3.714449537 | −1.773773894 | 5.488223431 |
| Hsd_65_r | 3.748646304 | −1.742543071 | 5.491189375 |
| HS_152_l | 3.102277233 | −2.393380365 | 5.495657598 |
| HSbrain_660_l | 3.466157655 | −2.029987544 | 5.496145198 |
| HS_from_MQbrain_1382_r | 3.13793393 | −2.363726857 | 5.501660787 |
| HS_181_r | 4.419469122 | −1.082251591 | 5.501720713 |
| HSbrain_805_l | 4.074433503 | −1.429676402 | 5.504109905 |
| Hsd_from_Mmd_516_l | 3.412475113 | −2.095033896 | 5.507509009 |
| Hsd_from_Mmd_245_l | 3.780690306 | −1.727121945 | 5.507812252 |
| HS_from_MQbrain_578_r | 4.166135797 | −1.350306755 | 5.516442553 |
| HSbrain_591_r | 4.391529413 | −1.12924627 | 5.520775683 |
| HSbrain_648_r | 3.194624354 | −2.333467448 | 5.528091801 |
| HS_from_PT_176_l | 3.956699706 | −1.576116801 | 5.532816508 |
| Hsd_7_l | 3.22704522 | −2.306215626 | 5.533260846 |
| HS_104_l | 3.942124617 | −1.592915077 | 5.535039694 |
| HSbrain_190_l | 4.758036339 | −0.780199851 | 5.53823619 |
| HS_from_MM_33_l | 3.840554748 | −1.69933794 | 5.539892688 |
| hsa-mir-19a_r | 1.9628064 | −3.57807505 | 5.540881449 |
| HSbrain_377_l | 2.436684747 | −3.106433448 | 5.543118196 |
| Hsd_from_Mmd_372_r | 4.246402876 | −1.299267958 | 5.545670834 |
| HS_from_MQbrain_1326_l | 3.976713406 | −1.572423915 | 5.549137321 |
| HSbrain_715_r | 3.453512886 | −2.09921914 | 5.552732026 |
| hsa-mir-26b_l | 1.764835795 | −3.788243571 | 5.553079366 |
| HS_from_MQbrain_594_r | 4.355831166 | −1.197465154 | 5.55329632 |
| HSbrain_653_r | 3.322388663 | −2.234544302 | 5.556932965 |
| hsa-mir-801_l | 4.017543096 | −1.544979742 | 5.562522838 |
| HS_from_MM_172_r | 1.397454629 | −4.169510735 | 5.566965364 |
| hsa-mir-124a-3_r | 4.617726819 | −0.952351832 | 5.57007865 |
| HSbrain_29_r | 4.260687702 | −1.309423869 | 5.570111571 |
| HS_from_MQbrain_1357_r | 4.306226408 | −1.270483357 | 5.576709765 |
| HSbrain_20_l | 3.751601424 | −1.849028269 | 5.600629694 |
| HS_from_PT_220_r | 4.281748809 | −1.320114272 | 5.601863081 |
| hsa-let-7f-2_l | 1.76029603 | −3.845246655 | 5.605542685 |
| HS_from_MQbrain_618_l | 3.445454811 | −2.164340001 | 5.609794812 |
| HSbrain_1405_l | 4.679472115 | −0.934087199 | 5.613559314 |
| Hsd_14_r | 5.086071816 | −0.528975621 | 5.615047436 |
| HSbrain_54_r | 4.294762627 | −1.32100822 | 5.615770847 |
| HSbrain_610_l | 3.599846841 | −2.015963198 | 5.615810039 |
| HS_from_MQbrain_1405_l | 3.775686066 | −1.842781398 | 5.618467464 |
| HS_21_l | 3.418371497 | −2.205431424 | 5.623802922 |
| HS_from_PT_219_r | 4.403612245 | −1.228598506 | 5.632210751 |
| HS_from_MQbrain_354_l | 4.735786231 | −0.899065708 | 5.63485194 |
| HS_from_MQbrain_918_r | 4.702403782 | −0.933235667 | 5.63563945 |
| HS_from_MQbrain_1061_r | 4.062583748 | −1.573537454 | 5.636121203 |
| hsa-mir-23a_r | 2.510329712 | −3.127381226 | 5.637710938 |
| Hsd_from_Mmd_266_r | 2.805780405 | −2.834756512 | 5.640536917 |
| HSbrain_411_r | 2.613717186 | −3.042395943 | 5.656113129 |
| HS_from_MQbrain_1646_l | 4.15001229 | −1.523739312 | 5.673751602 |
| HSbrain_317_r | 3.162439297 | −2.516972317 | 5.679411615 |
| hsa-mir-31_l | 3.922440913 | −1.765548382 | 5.687989296 |
| HS_90_l | 4.227924969 | −1.461704043 | 5.689629012 |
| HS_183_l | 3.456161799 | −2.234645299 | 5.690807098 |
| HSbrain_99_l | 3.920364938 | −1.770561757 | 5.690926695 |
| HS_from_MM_47_r | 3.671346828 | −2.024788693 | 5.696135521 |
| hsa-mir-572_r | 4.971923816 | −0.727695707 | 5.699619523 |
| Hsd_from_Mmd_115_l | 4.853790974 | −0.852245434 | 5.706036407 |
| HS_from_MQbrain_796_r | 4.415678039 | −1.29112765 | 5.706805689 |
| HS_from_MQbrain_433_r | 4.108677086 | −1.600787572 | 5.709464658 |
| hsa-mir-181c_l | 2.138737177 | −3.573048009 | 5.711785186 |
| Hsd_from_Mmd_154_r | 3.762836127 | −1.949146778 | 5.711982905 |
| Hsd_33_l | 3.343345301 | −2.369363954 | 5.712709255 |
| HSbrain_499_l | 3.469097037 | −2.246893498 | 5.715990535 |
| HS_from_MQbrain_1362_r | 4.28548566 | −1.435541542 | 5.721027201 |
| HS_from_MM_210_l | 4.10523656 | −1.617132807 | 5.722369367 |
| hsa-mir-519e_r | 3.116430575 | −2.612911942 | 5.729342517 |
| HS_72_l | 4.268290644 | −1.463946628 | 5.732237271 |
| HS_from_PT_125_l | 4.702104541 | −1.033066802 | 5.735171343 |
| HSbrain_1389_l | 3.024112414 | −2.711581946 | 5.735694359 |
| HS_from_MQbrain_273_r | 4.632780656 | −1.124780689 | 5.757561345 |
| hsa-mir-124a-2_r | 4.828261068 | −0.933932683 | 5.762193751 |
| HSbrain_327_r | 4.660529086 | −1.10899521 | 5.769524296 |
| HSbrain_482_l | 4.199508553 | −1.575202359 | 5.774710912 |
| Hsd_from_Mmd_35_r | 4.487620932 | −1.295421708 | 5.78304264 |
| HSbrain_40_l | 4.019944325 | −1.773412253 | 5.793356578 |
| HSbrain_533_r | 4.196700156 | −1.619525068 | 5.816225224 |
| HS_from_MQbrain_1858_r | 4.483323757 | −1.335052328 | 5.818376085 |
| hsa-let-7g_l | 1.806582222 | −4.012710072 | 5.819292294 |
| HS_from_MM_35_r | 4.703256578 | −1.11746028 | 5.820716859 |
| HSbrain_112_l | 3.235279252 | −2.586285347 | 5.821564599 |
| Hsd_from_Mmd_246_r | 2.780871742 | −3.043528889 | 5.824400631 |
| HSbrain_28_r | 5.48605974 | −0.346305969 | 5.832365709 |
| hsa-mir-566_l | 3.429280027 | −2.403734308 | 5.833014335 |
| HS_from_MQbrain_924_r | 3.811192391 | −2.037005336 | 5.848197727 |
| HS_from_MQbrain_575_l | 4.830160754 | −1.01981064 | 5.849971394 |
| Hsd_72_l | 3.836626412 | −2.014491701 | 5.851118113 |
| HSbrain_377_r | 2.907687708 | −2.947146756 | 5.854834465 |
| HSbrain_657_r | 4.303051294 | −1.556854409 | 5.859905704 |
| HSbrain_26_r | 4.236057005 | −1.63051195 | 5.866568955 |
| hsa-mir-200c_r | 2.576005313 | −3.290828708 | 5.866834021 |
| HS_from_MM_296_l | 4.744357804 | −1.129429551 | 5.873787355 |
| HS_267_r | 3.354881307 | −2.520776419 | 5.875657726 |
| HS_from_MQbrain_732_l | 5.596275896 | −0.283871028 | 5.880146923 |
| HSbrain_418_r | 4.272110494 | −1.60831359 | 5.880424085 |
| HS_from_MQbrain_2633_l | 3.794994035 | −2.096595326 | 5.891589361 |
| HSbrain_278_r | 3.805184267 | −2.089225652 | 5.894409919 |
| HSbrain_558_l | 4.291784938 | −1.602898464 | 5.894683402 |
| HS_143_r | 3.012115136 | −2.894275789 | 5.906390924 |
| HS_from_MQbrain_792_r | 3.643333503 | −2.281909887 | 5.92524339 |
| HS_14_l | 4.709045762 | −1.218830057 | 5.927875819 |
| HS_118_l | 3.878595746 | −2.054769168 | 5.933364914 |
| HS_from_MQbrain_8_r | 3.772568617 | −2.162192464 | 5.934761082 |
| hsa-mir-23b_r | 2.780138092 | −3.160150843 | 5.940288935 |
| HS_from_PT_7_r | 4.737928104 | −1.206096838 | 5.944024942 |
| Hsd_from_Mmd_260_l | 4.790164575 | −1.156684188 | 5.946848763 |
| HSbrain_654_l | 4.975188549 | −0.97202554 | 5.947214089 |
| HSbrain_15_l | 4.679770369 | −1.269776956 | 5.949547325 |
| HS_from_MM_234_l | 4.240768704 | −1.717054897 | 5.957823601 |
| hsa-mir-143_r | 4.421696799 | −1.538445702 | 5.960142501 |
| HS_from_MQbrain_760_r | 5.809892718 | −0.155311287 | 5.965204005 |
| hsa-mir-542_r | 4.473592048 | −1.497936368 | 5.971528416 |
| hsa-mir-124a-1_r | 5.075346959 | −0.896800495 | 5.972147454 |
| HS_from_MM_29_l | 4.117736752 | −1.854487591 | 5.972224343 |
| HS_192_r | 4.989345344 | −0.993611356 | 5.9829567 |
| HS_254_r | 4.114536249 | −1.868510953 | 5.983047203 |
| HS_from_MQbrain_843_l | 4.352681523 | −1.63782679 | 5.990508313 |
| HS_193_l | 4.75212295 | −1.239024261 | 5.99114721 |
| HS_from_PT_130_r | 3.148538054 | −2.856593123 | 6.005131177 |
| HSbrain_765_r | 4.882015038 | −1.128191572 | 6.01020661 |
| HS_from_MQbrain_1421_r | 3.888984872 | −2.124894931 | 6.013879803 |
| HSbrain_436_l | 4.30323071 | −1.715247803 | 6.018478513 |
| HS_287_r | 4.495264347 | −1.534503365 | 6.029767713 |
| HSbrain_499_r | 4.098369288 | −1.932646304 | 6.031015592 |
| hsa-mir-564_r | 4.909851444 | −1.123461194 | 6.033312638 |
| HSbrain_208_r | 3.212598106 | −2.830561144 | 6.043159249 |
| HS_from_MQbrain_807_l | 4.661135153 | −1.382077972 | 6.043213126 |
| Hsd_from_Mmd_222_r | 3.32302302 | −2.721408253 | 6.044431273 |
| HSbrain_1360_r | 4.547229214 | −1.500155717 | 6.047384931 |
| Hsd_from_Mmd_271_r | 3.42090646 | −2.633339035 | 6.054245494 |
| hsa-mir-636_l | 4.586138795 | −1.480297143 | 6.066435938 |
| HSbrain_83_r | 5.045353177 | −1.02436812 | 6.069721297 |
| HS_from_MQbrain_452_l | 3.931878227 | −2.142948942 | 6.07482717 |
| HS_from_MQbrain_661_l | 3.309819773 | −2.767618454 | 6.077438227 |
| HSbrain_111_l | 4.478063253 | −1.601864592 | 6.079927845 |
| HS_from_MQbrain_1205_r | 3.798533287 | −2.287026649 | 6.085559937 |
| HS_from_PT_123_r | 4.699877455 | −1.393673208 | 6.093550663 |
| HS_from_MQbrain_1803_l | 3.990717912 | −2.104101824 | 6.094819736 |
| hsa-mir-208_l | 4.498924031 | −1.604037691 | 6.102961722 |
| HSbrain_238_l | 4.522924806 | −1.581116125 | 6.104040931 |
| HS_from_MQbrain_137_l | 5.249572176 | −0.855933068 | 6.105505244 |
| HSbrain_1363_r | 4.690028685 | −1.419093516 | 6.109122201 |
| HS_from_MM_260_r | 3.447152671 | −2.667288315 | 6.114440986 |
| hsa-mir-30e_l | 3.0644184 | −3.050098809 | 6.11451721 |
| HSbrain_16_l | 4.573890866 | −1.550435178 | 6.124326044 |
| HSbrain_122_r | 4.320735556 | −1.818946269 | 6.139681825 |
| hsa-mir-551a_r | 4.680313939 | −1.465502125 | 6.145816064 |
| hsa-mir-520f_l | 5.79689907 | −0.350966934 | 6.147866004 |
| HSbrain_394_l | 4.573621055 | −1.590725756 | 6.164346812 |
| HSbrain_375_l | 4.170862628 | −1.99497765 | 6.165840277 |
| HS_from_MQbrain_287_r | 4.390750308 | −1.775092716 | 6.165843023 |
| Hsd_26_r | 4.528037162 | −1.646247286 | 6.174284449 |
| HSbrain_131_r | 4.642917681 | −1.531943569 | 6.17486125 |
| HS_from_MQbrain_1502_l | 4.306880119 | −1.872571169 | 6.179451288 |
| HS_117_l | 3.914149118 | −2.266893911 | 6.181043029 |
| HS_from_MQbrain_374_l | 4.048069346 | −2.135713316 | 6.183782662 |
| HSbrain_366_r | 4.695428359 | −1.500832349 | 6.196260708 |
| HS_11_r | 4.359066265 | −1.840949358 | 6.200015623 |
| HSbrain_766_r | 4.292091907 | −1.907936735 | 6.200028642 |
| Hsd_from_Mmd_433_l | 4.189641245 | −2.014185933 | 6.203827179 |
| HS_288_r | 4.613651482 | −1.601155032 | 6.214806514 |
| Hsd_92_l | 4.453319016 | −1.772517608 | 6.225836624 |
| HS_from_MQbrain_1295_l | 4.467230834 | −1.769790663 | 6.237021496 |
| hsa-mir-498_r | 4.742805113 | −1.497579431 | 6.240384544 |
| HS_from_MQbrain_1312_l | 3.945332299 | −2.307158367 | 6.252490666 |
| HS_from_MQbrain_521_r | 4.250672499 | −2.008401215 | 6.259073714 |
| HS_114_l | 4.014399822 | −2.260096796 | 6.274496618 |
| HSbrain_383_l | 2.848711871 | −3.430707313 | 6.279419184 |
| HSbrain_653_l | 3.553247415 | −2.727155834 | 6.280403249 |
| HS_from_MQbrain_1765_l | 4.776879998 | −1.506865206 | 6.283745204 |
| hsa-mir-363_l | 4.650693847 | −1.643106855 | 6.293800702 |
| HS_from_MQbrain_1065_l | 4.432098881 | −1.863369953 | 6.295468834 |
| HSbrain_706_l | 4.714094744 | −1.583427201 | 6.297521946 |
| Hsd_from_Mmd_59_r | 4.939409317 | −1.365011536 | 6.304420853 |
| HS_205_r | 4.953281694 | −1.358308462 | 6.311590155 |
| HS_206_l | 3.947577057 | −2.36586125 | 6.313438307 |
| hsa-mir-106b_l | 2.230048384 | −4.084607376 | 6.314655761 |
| hsa-mir-375_r | 3.404471243 | −2.913526702 | 6.317997945 |
| hsa-mir-612_r | 4.153701447 | −2.164532199 | 6.318233646 |
| Hsd_from_Mmd_227_r | 4.969514785 | −1.350466056 | 6.319980841 |
| HS_from_MQbrain_525_l | 4.325248296 | −2.001844685 | 6.327092981 |
| HS_284_r | 4.382098264 | −1.948138028 | 6.330236293 |
| hsa-mir-27a_r | 1.963166985 | −4.378793384 | 6.341960369 |
| HSbrain_42_r | 3.597747739 | −2.746338563 | 6.344086302 |
| HSbrain_118_l | 5.091238684 | −1.260477419 | 6.351716103 |
| HS_from_MQbrain_1110_r | 4.905874678 | −1.456293927 | 6.362168605 |
| HS_from_PT_63_l | 4.729838898 | −1.633775996 | 6.363614894 |
| HSbrain_484_l | 3.495467707 | −2.874483359 | 6.369951066 |
| HS_from_MQbrain_820_l | 5.400175183 | −0.969934259 | 6.370109442 |
| HS_from_MQbrain_601_l | 4.33308059 | −2.041207019 | 6.374287609 |
| HS_from_MQbrain_1570_r | 5.826121312 | −0.554136646 | 6.380257959 |
| HS_212_r | 2.969552561 | −3.426937067 | 6.396489629 |
| hsa-mir-551b_r | 5.02160857 | −1.381305208 | 6.402913778 |
| hsa-mir-517a_l | 4.530169487 | −1.873171691 | 6.403341178 |
| HSbrain_214_l | 4.736730898 | −1.66726547 | 6.403996368 |
| HS_302_l | 4.597912524 | −1.826783726 | 6.42469625 |
| HS_from_MQbrain_630_l | 4.231929713 | −2.193260438 | 6.425190151 |
| HSbrain_20_r | 5.062760753 | −1.364215928 | 6.426976681 |
| HS_from_PT_104_r | 5.278512668 | −1.162086911 | 6.440599579 |
| hsa-mir-200b_r | 2.909157847 | −3.54818764 | 6.457345488 |
| HS_from_MQbrain_967_l | 4.266510532 | −2.202403708 | 6.46891424 |
| HS_257_r | 5.232858698 | −1.254197891 | 6.48705659 |
| Hsd_from_Mmd_137_r | 4.206897564 | −2.282544453 | 6.489442017 |
| HS_82_r | 4.799299574 | −1.691810282 | 6.491109856 |
| HSbrain_566_l | 4.66772169 | −1.83147388 | 6.499195571 |
| HS_from_MQbrain_1793_l | 4.095637227 | −2.40403094 | 6.499668167 |
| HS_273_l | 4.833637268 | −1.671078137 | 6.504715405 |
| HS_100_l | 3.193876747 | −3.31976598 | 6.513642727 |
| hsa-mir-29a_r | 2.028711838 | −4.495005517 | 6.523717354 |
| HS_from_PT_155_l | 5.059131261 | −1.466536807 | 6.525668067 |
| HS_from_PT_104_l | 4.4602838 | −2.066362192 | 6.526645992 |
| HSbrain_28_l | 5.393049923 | −1.139202058 | 6.532251981 |
| HS_from_PT_221_r | 4.355040962 | −2.180277912 | 6.535318874 |
| HSbrain_1362_r | 4.944687989 | −1.59084435 | 6.535532339 |
| HS_from_MQbrain_1777_r | 5.150932182 | −1.387694907 | 6.538627089 |
| hsa-mir-19b-2_r | 1.989061638 | −4.549799434 | 6.538861071 |
| HS_from_MQbrain_144_l | 4.038471969 | −2.503355258 | 6.541827227 |
| HS_from_MQbrain_1144_l | 3.758683069 | −2.80260379 | 6.56128686 |
| hsa-mir-484_r | 5.579400105 | −0.985905105 | 6.56530521 |
| HS_from_MQbrain_537_l | 4.746677785 | −1.819698903 | 6.566376688 |
| HS_from_PT_180_r | 4.902484378 | −1.667109583 | 6.569593961 |
| HS_from_MM_83_l | 4.066456218 | −2.51897756 | 6.585433779 |
| HS_289_r | 5.108709649 | −1.481421146 | 6.590130794 |
| HS_from_MQbrain_462_l | 4.64779877 | −1.957301099 | 6.605099869 |
| hsa-mir-106a_l | 2.12180599 | −4.487431834 | 6.609237824 |
| HSbrain_526_l | 4.573914512 | −2.037321142 | 6.611235653 |
| hsa-mir-20b_l | 2.021731933 | −4.59922435 | 6.620956283 |
| HSbrain_806_r | 4.240528032 | −2.384542882 | 6.625070914 |
| HS_from_MM_323_r | 5.480266936 | −1.14677998 | 6.627046915 |
| Hsd_11_r | 5.249599651 | −1.383955949 | 6.6335556 |
| hsa-mir-141_r | 3.847321923 | −2.789655843 | 6.636977766 |
| HS_from_MQbrain_207_r | 4.838454137 | −1.807132718 | 6.645586855 |
| HS_from_MQbrain_1175_r | 4.195815982 | −2.452473053 | 6.648289034 |
| HS_from_MQbrain_1712_l | 4.569203391 | −2.079799264 | 6.649002655 |
| hsa-mir-517c_l | 4.491318049 | −2.1589711 | 6.650289149 |
| HS_from_PT_269_l | 4.12142642 | −2.531640317 | 6.653066737 |
| HSbrain_638_l | 4.928462908 | −1.742445994 | 6.670908902 |
| HS_from_MQbrain_477_l | 4.941037179 | −1.737334585 | 6.678371764 |
| HSbrain_644_l | 5.690934417 | −0.990262022 | 6.681196439 |
| HS_from_MQbrain_1090_l | 4.563380193 | −2.118979823 | 6.682360015 |
| HS_from_MQbrain_1571_r | 5.33383266 | −1.354822809 | 6.688655469 |
| hsa-mir-26a-1_l | 2.86738772 | −3.823840736 | 6.691228456 |
| HS_from_MQbrain_506_l | 4.997959927 | −1.694546909 | 6.692506836 |
| hsa-mir-19b-1_r | 2.111928811 | −4.594002685 | 6.705931496 |
| HSbrain_137_l | 4.893237752 | −1.816706861 | 6.709944614 |
| hsa-mir-517b_l | 4.566645976 | −2.145699396 | 6.712345372 |
| HS_from_MQbrain_140_r | 3.986572678 | −2.726394722 | 6.7129674 |
| HS_from_MQbrain_16_r | 5.566280457 | −1.148896248 | 6.715176705 |
| HS_from_MQbrain_1539_l | 4.876671232 | −1.847412856 | 6.724084088 |
| HS_from_MQbrain_552_r | 5.017813786 | −1.716953375 | 6.734767161 |
| HS_290_r | 5.182365159 | −1.562217353 | 6.744582512 |
| hsa-mir-20a_l | 2.062165027 | −4.684878044 | 6.747043071 |
| hsa-mir-17_l | 2.126869394 | −4.625109005 | 6.751978399 |
| HSbrain_750_r | 4.066569697 | −2.700350055 | 6.766919752 |
| HS_from_MQbrain_340_r | 4.435814155 | −2.341736847 | 6.777551002 |
| hsa-mir-26a-2_l | 3.040722248 | −3.737428702 | 6.77815095 |
| HS_from_MQbrain_17_r | 4.479251469 | −2.303997472 | 6.783248941 |
| HSbrain_332_l | 5.213396221 | −1.578722089 | 6.79211831 |
| Hsd_from_Mmd_483_r | 5.315092406 | −1.483685329 | 6.798777735 |
| HS_from_MQbrain_1728_l | 4.985635056 | −1.822843757 | 6.808478813 |
| HS_from_MQbrain_340_l | 3.895756709 | −2.917362468 | 6.813119177 |
| HS_from_MQbrain_279_l | 5.214245442 | −1.60627643 | 6.820521872 |
| HSbrain_438_l | 4.738087688 | −2.085318354 | 6.823406042 |
| HS_from_MM_129_l | 4.874406145 | −1.951318632 | 6.825724776 |
| HS_from_MQbrain_1201_l | 4.276418531 | −2.551107134 | 6.827525665 |
| HS_from_MQbrain_1497_r | 4.97080598 | −1.859203898 | 6.830009878 |
| hsa-mir-200a_r | 3.963877308 | −2.86637397 | 6.830251278 |
| HS_from_MQbrain_1320_r | 5.103524221 | −1.729838067 | 6.833362287 |
| Hsd_55_l | 4.531891327 | −2.302100667 | 6.833991994 |
| HSbrain_660_l | 5.513419211 | −1.330781278 | 6.844200489 |
| HSbrain_1364_r | 5.366606756 | −1.485438789 | 6.852045545 |
| hsa-mir-593_l | 4.932339535 | −1.925441211 | 6.857780746 |
| HS_231_l | 4.686130131 | −2.174007413 | 6.860137544 |
| hsa-mir-29c_r | 2.178692982 | −4.681702494 | 6.860395475 |
| HSbrain_437_l | 4.762473053 | −2.124418959 | 6.886892012 |
| HS_250_l | 4.137454636 | −2.750116172 | 6.887570808 |
| HS_from_MQbrain_153_l | 5.873972902 | −1.014588707 | 6.888561609 |
| hsa-mir-557_l | 5.733681079 | −1.162104113 | 6.895785191 |
| HSbrain_758_l | 5.103685318 | −1.794979992 | 6.898665311 |
| HS_from_MQbrain_453_l | 4.787359609 | −2.13175075 | 6.919110359 |
| HS_from_PT_292_l | 4.552921488 | −2.386407281 | 6.93932877 |
| hsa-mir-560_l | 4.394413378 | −2.551856773 | 6.946270152 |
| hsa-mir-345_r | 4.273594511 | −2.678761116 | 6.952355627 |
| HS_from_MQbrain_279_r | 4.939079839 | −2.042983141 | 6.98206298 |
| HS_from_MQbrain_809_l | 4.985103282 | −2.011053263 | 6.996156544 |
| HS_from_MM_147_l | 3.743097086 | −3.261243741 | 7.004340828 |
| HS_from_MQbrain_1843_l | 5.965959445 | −1.052555568 | 7.018515013 |
| HS_from_MQbrain_622_l | 4.376171362 | −2.645175527 | 7.021346889 |
| HS_from_MQbrain_606_r | 4.756545126 | −2.283771606 | 7.040316732 |
| HSbrain_582_r | 3.689488954 | −3.354690718 | 7.044179672 |
| HS_from_MQbrain_1463_l | 4.880859335 | −2.164240124 | 7.045099459 |
| HSbrain_136_r | 5.079441797 | −1.971376246 | 7.050818042 |
| HS_from_MQbrain_2630_l | 4.398935251 | −2.674677908 | 7.073613159 |
| HSbrain_72_r | 6.017630146 | −1.069678172 | 7.087308318 |
| HSbrain_587_l | 5.1299973 | −1.964544846 | 7.094542146 |
| HS_from_MQbrain_973_l | 4.943200444 | −2.172290006 | 7.11549045 |
| HS_from_MQbrain_1168_l | 4.97862079 | −2.137842327 | 7.116463118 |
| Hsd_from_Mmd_426_l | 3.747335182 | −3.370219535 | 7.117554717 |
| hsa-mir-663_l | 5.011240432 | −2.159387436 | 7.170627867 |
| hsa-mir-517c_r | 4.480986651 | −2.69422913 | 7.175215781 |
| HSbrain_237_r | 4.675472342 | −2.51110095 | 7.186573292 |
| HS_from_MQbrain_1404_l | 4.05761419 | −3.139556758 | 7.197170948 |
| Hsd_from_Mmd_74_l | 4.339313831 | −2.867371228 | 7.206685059 |
| HS_from_MQbrain_170_l | 6.138058127 | −1.080821964 | 7.218880091 |
| HS_from_MM_329_r | 5.712011478 | −1.521907738 | 7.233919216 |
| HS_from_MQbrain_1497_l | 6.073170949 | −1.174094228 | 7.247265177 |
| HS_from_MQbrain_1810_r | 6.122979468 | −1.126623894 | 7.249603362 |
| HS_from_MQbrain_808_l | 4.947162374 | −2.309157784 | 7.256320158 |
| Hsd_from_Mmd_286_r | 4.226672233 | −3.037609517 | 7.26428175 |
| HS_from_MQbrain_671_l | 5.534101291 | −1.744414628 | 7.278515919 |
| HS_from_MQbrain_1195_l | 5.519733146 | −1.778822251 | 7.298555397 |
| HS_from_MQbrain_137_r | 4.292665749 | −3.02271647 | 7.315382219 |
| HS_from_MQbrain_453_r | 5.096022508 | −2.234501088 | 7.330523596 |
| HSbrain_620_l | 4.619780262 | −2.722968038 | 7.3427483 |
| HS_from_MQbrain_800_l | 4.99053162 | −2.358874546 | 7.349406166 |
| hsa-mir-29b-1_r | 2.507736393 | −4.843123724 | 7.350860117 |
| hsa-mir-565_l | 4.149892881 | −3.206795324 | 7.356688205 |
| HSbrain_468_l | 3.80362517 | −3.563185747 | 7.366810918 |
| hsa-mir-29b-2_r | 2.563842324 | −4.826456007 | 7.390298331 |
| HSbrain_534_l | 4.500081575 | −2.894021126 | 7.394102701 |
| HS_from_MQbrain_1109_l | 4.029606516 | −3.382276204 | 7.41188272 |
| HS_130_l | 6.21383474 | −1.223177365 | 7.437012105 |
| HS_213_r | 3.448098231 | −4.004380141 | 7.452478372 |
| HS_from_MQbrain_1784_r | 5.458060554 | −2.008655821 | 7.466716375 |
| hsa-mir-92-1_r | 3.953740575 | −3.523815432 | 7.477556007 |
| HS_from_MQbrain_681_r | 4.86756847 | −2.655295001 | 7.522863471 |
| HS_from_MQbrain_1030_r | 5.482419864 | −2.050160962 | 7.532580827 |
| HSbrain_1337_r | 3.552234375 | −3.996126137 | 7.548360511 |
| HS_from_MQbrain_386_r | 4.667495409 | −2.88737231 | 7.554867719 |
| HSbrain_609_r | 5.007422508 | −2.616095825 | 7.623518334 |
| hsa-mir-92-2_r | 3.874690749 | −3.760803493 | 7.635494241 |
| hsa-mir-663_r | 5.467271847 | −2.171521464 | 7.638793311 |
| HS_from_MQbrain_485_l | 4.276180188 | −3.36267375 | 7.638853938 |
| HS_from_MQbrain_530_l | 4.530899577 | −3.136538969 | 7.667438546 |
| HS_from_MQbrain_1590_r | 5.618889763 | −2.063583825 | 7.682473587 |
| HSbrain_412_l | 4.076386045 | −3.674915919 | 7.751301964 |
| hsa-mir-9-2_r | 4.593661545 | −3.177024616 | 7.770686161 |
| HS_from_MQbrain_666_l | 5.485658527 | −2.32972148 | 7.815380007 |
| HS_from_MQbrain_1490_l | 5.64200061 | −2.257253419 | 7.899254029 |
| hsa-mir-9-1_r | 4.700973627 | −3.281093675 | 7.982067302 |
| hsa-mir-9-3_r | 4.729775968 | −3.255497845 | 7.985273812 |
| HS_from_PT_114_l | 3.583990998 | −4.407945806 | 7.991936804 |
| HS_from_MQbrain_868_l | 4.38199392 | −3.612346682 | 7.994340602 |
| HS_from_MQbrain_750_l | 4.24585762 | −3.788058659 | 8.033916279 |
| HSbrain_225_l | 5.102922553 | −3.13165277 | 8.234575323 |
| HS_from_PT_114_r | 3.967307291 | −4.292042281 | 8.259349572 |
| HS_68_l | 4.385354801 | −3.937554066 | 8.322908867 |
| hsa-mir-520g_l | 4.943810712 | −3.467378187 | 8.4111889 |
| HS_from_MM_303_r | 4.847876578 | −3.649650041 | 8.497526619 |
| HS_306_l | 5.653094519 | −2.863407345 | 8.516501864 |
| Hsd_from_Mmd_383_r | 4.846843191 | −3.732116805 | 8.578959996 |
| HS_306_l | 5.556294478 | −3.05973905 | 8.616033528 |
| hsa-mir-145_l | 5.644247849 | −3.038892211 | 8.683140061 |
| Hsd_from_Mmd_94_l | 4.90141117 | −3.801059753 | 8.702470924 |
| Hsd_13_l | 5.772543113 | −2.933788243 | 8.706331355 |
| HS_from_MQbrain_817_l | 5.668999177 | −3.046828158 | 8.715827335 |
| HS_from_PT_206_l | 4.727701851 | −4.018056539 | 8.74575839 |
| hsa-mir-520h_l | 5.131084114 | −3.655170271 | 8.786254385 |
| hsa-mir-671_l | 5.767779447 | −3.053619735 | 8.821399182 |
| hsa-mir-21_l | 1.609681489 | −7.236732615 | 8.846414105 |
| HS_from_PT_313_l | 5.481942633 | −3.408145534 | 8.890088167 |
| HS_from_MQbrain_722_r | 5.410709411 | −3.573201166 | 8.983910577 |
| HSbrain_126_r | 5.454802988 | −3.86635451 | 9.321157497 |

¹Cut off log2 range > 4

TABLE 2a
Small RNAs that are differentially expressed between control and
adenoma

| | adenoma vs |
|---|---|
| Systematic Name | control adj p¹ |
| HS_from_MM_118_r | 0.000489293 |
| HS_from_MM_169_r | 0.000489293 |
| HSbrain_106_r | 0.000489293 |
| HSbrain_801_r | 0.000489293 |
| Hsd_from_Mmd_485_r | 0.000489293 |
| Hsd_from_Mmd_452_l | 0.000704567 |
| hsa-mir-130a_r | 0.001129352 |
| HSbrain_731_r | 0.001129352 |
| HS_from_MQbrain_1311_r | 0.001446314 |
| hsa-mir-655_l | 0.001446314 |
| HS_from_MQbrain_1297_l | 0.001647426 |
| HSbrain_458_r | 0.001883183 |
| HS_from_MQbrain_1292_r | 0.003939952 |
| HS_from_MQbrain_1657_r | 0.003939952 |
| HS_from_MQbrain_839_r | 0.003939952 |
| HS_from_PT_36_l | 0.003939952 |
| HS_79_l | 0.004006907 |
| HS_from_MQbrain_100_l | 0.004006907 |
| HS_from_MQbrain_1183_l | 0.004006907 |
| HS_from_MQbrain_1363_l | 0.004006907 |
| HS_from_MQbrain_1628_r | 0.004006907 |
| HS_from_MQbrain_1823_r | 0.004006907 |
| hsa-mir-633_l | 0.004006907 |
| HS_from_MQbrain_288_r | 0.004431894 |
| HSbrain_377_l | 0.004431894 |
| HS_from_MQbrain_131_r | 0.004575487 |
| HS_from_MQbrain_20_r | 0.004575487 |
| HS_from_MQbrain_212_r | 0.004575487 |
| hsa-mir-125a_r | 0.004575487 |
| hsa-mir-196a-2_r | 0.004799641 |
| hsa-mir-302d_l | 0.004799641 |
| hsa-mir-497_r | 0.004799641 |
| HSbrain_629_l | 0.004799641 |
| HS_from_MQbrain_1070_r | 0.004957702 |
| HS_from_MQbrain_2380_r | 0.004957702 |
| hsa-mir-616_r | 0.004957702 |
| HSbrain_52_r | 0.004957702 |
| Hsd_from_Mmd_480_l | 0.004957702 |
| hsa-mir-421_r | 0.006601644 |
| HSbrain_224_r | 0.006601644 |
| HS_from_MM_339_r | 0.00676609 |
| HS_from_MQbrain_566_l | 0.00676609 |
| hsa-mir-554_l | 0.00676609 |
| hsa-mir-573_l | 0.00676609 |
| HSbrain_1076_r | 0.00676609 |
| HSbrain_426_r | 0.00676609 |
| HSbrain_102_r | 0.007781761 |
| HS_from_MQbrain_441_l | 0.008573112 |
| HSbrain_231_l | 0.008573112 |
| Hsd_49_r | 0.008573112 |
| HS_217_l | 0.008946182 |
| HS_from_MM_295_l | 0.008946182 |
| HS_from_MQbrain_1485_r | 0.008946182 |
| HS_from_MQbrain_1522_r | 0.008946182 |
| HS_from_PT_101_r | 0.008946182 |
| Hsd_from_Mmd_298_r | 0.008946182 |
| HS_141_l | 0.009734613 |
| HS_71_l | 0.009734613 |
| HSbrain_411_r | 0.009734613 |
| Hsd_from_Mmd_117_r | 0.009734613 |
| HS_291_r | 0.01044887 |
| HS_from_MQbrain_1462_l | 0.01044887 |
| hsa-mir-325_l | 0.01044887 |
| HSbrain_483_l | 0.01044887 |
| HSbrain_754_r | 0.01044887 |
| HS_215_r | 0.010727702 |
| HS_from_MQbrain_1056_l | 0.010727702 |
| HS_from_MQbrain_655_l | 0.010727702 |
| HS_from_MQbrain_671_r | 0.010727702 |
| hsa-let-7a-3_l | 0.010727702 |
| hsa-mir-135a-2_r | 0.010727702 |
| hsa-mir-516-3_l | 0.010727702 |
| hsa-mir-659_l | 0.010727702 |
| hsa-mir-768_r | 0.010727702 |
| hsa-mir-98_l | 0.010727702 |
| HSbrain_253_l | 0.010727702 |
| HSbrain_268_r | 0.010727702 |
| HSbrain_337_l | 0.010727702 |
| HSbrain_398_r | 0.010727702 |
| HSbrain_472_l | 0.010727702 |
| HSbrain_485_r | 0.010727702 |
| HSbrain_583_l | 0.010727702 |
| HSbrain_727_l | 0.010727702 |
| HSbrain_794_r | 0.010727702 |
| HSbrain_811_r | 0.010727702 |
| HS_232_l | 0.011699489 |
| HS_from_MQbrain_629_l | 0.011699489 |
| HSbrain_359_r | 0.011699489 |
| HSbrain_709_l | 0.011699489 |
| HSbrain_916_l | 0.011699489 |
| HS_169_l | 0.012506309 |
| HS_from_MQbrain_194_l | 0.012506309 |
| HS_from_MQbrain_330_r | 0.012506309 |
| HSbrain_1437_r | 0.012506309 |
| HSbrain_722_l | 0.012506309 |
| Hsd_from_Mmd_155_l | 0.012506309 |
| Hsd_from_Mmd_219_r | 0.012506309 |
| HS_from_MQbrain_1687_l | 0.013034759 |
| HS_from_MQbrain_71_r | 0.013034759 |
| HS_from_MQbrain_843_r | 0.013034759 |
| hsa-let-7f-1_l | 0.013034759 |
| hsa-let-7g_l | 0.013034759 |
| hsa-mir-126_r | 0.013034759 |
| hsa-mir-516-4_l | 0.013034759 |
| HSbrain_399_l | 0.013034759 |
| HSbrain_617_l | 0.013034759 |
| Hsd_107_l | 0.013034759 |
| HS_from_MQbrain_1444_l | 0.014035635 |
| HS_from_MQbrain_79_r | 0.014035635 |
| HSbrain_2_l | 0.014035635 |
| HSbrain_377_r | 0.014035635 |
| HSbrain_446_l | 0.014035635 |
| HSbrain_75_l | 0.014035635 |
| Hsd_70_l | 0.014035635 |
| HS_9_r | 0.015394932 |
| HS_from_MQbrain_1485_l | 0.015394932 |
| HS_from_MQbrain_349_l | 0.015394932 |
| HSbrain_1305_l | 0.015394932 |
| HSbrain_540_r | 0.015394932 |
| HS_21_r | 0.015739043 |
| HS_from_MQbrain_1624_l | 0.015739043 |
| HS_from_MQbrain_1792_r | 0.015739043 |
| HS_from_MQbrain_191_l | 0.015739043 |
| HS_from_MQbrain_2700_r | 0.015739043 |
| HS_from_MQbrain_375_l | 0.015739043 |
| HS_from_MQbrain_619_r | 0.015739043 |
| HS_from_MQbrain_940_r | 0.015739043 |
| hsa-mir-133a-2_r | 0.015739043 |
| hsa-mir-181a-1_l | 0.015739043 |
| hsa-mir-591_r | 0.015739043 |
| HSbrain_12_l | 0.015739043 |
| HSbrain_753_r | 0.015739043 |
| Hsd_from_Mmd_473_l | 0.015739043 |
| HS_281_r | 0.016464393 |
| HS_from_mM_264_l | 0.016464393 |
| HS_from_MQbrain_350_l | 0.016464393 |
| HS_from_MQbrain_825_r | 0.016464393 |
| HS_from_MQbrain_854_r | 0.016464393 |
| HS_from_PT_114_r | 0.016464393 |
| hsa-let-7a-1_l | 0.016464393 |
| hsa-Iet-7a-2_l | 0.016464393 |
| HSbrain_1_l | 0.016464393 |
| HSbrain_158_l | 0.016464393 |
| HSbrain_171_l | 0.016464393 |
| HSbrain_420_r | 0.016464393 |
| HS_from_MQbrain_1100_l | 0.017198956 |
| HS_from_MQbrain_1128_l | 0.017198956 |
| HS_from_MQbrain_1393_r | 0.017198956 |
| HS_from_MQbrain_1630_l | 0.017198956 |
| HS_from_MQbrain_965_l | 0.017198956 |
| HS_from_PT_190_r | 0.017198956 |
| hsa-mir-181c_l | 0.017198956 |
| hsa-mir-296_l | 0.017198956 |
| HSbrain_148_r | 0.017198956 |
| HSbrain_225_r | 0.017198956 |
| HSbrain_713_r | 0.017198956 |
| Hsd_18_l | 0.017198956 |
| Hsd_from_Mmd_519_l | 0.017198956 |
| HS_from_MM_39_l | 0.018600058 |
| HS_from_MQbrain_1625_r | 0.018600058 |
| hsa-mir-454_r | 0.018600058 |
| HSbrain_398_l | 0.018600058 |
| HSbrain_636_l | 0.018600058 |
| HSbrain_703_r | 0.018600058 |
| Hsd_from_Mmd_533_l | 0.018600058 |
| Hsd_from_Mmd_92_r | 0.018600058 |
| HS_262_l | 0.019454312 |
| HS_from_MM_309_r | 0.019454312 |
| HS_from_MQbrain_1103_l | 0.019454312 |
| HS_from_MQbrain_917_r | 0.019454312 |
| HS_from_PT_146_r | 0.019454312 |
| HS_from_PT_40_r | 0.019454312 |
| hsa-mir-299_l | 0.019454312 |
| hsa-mir-568_r | 0.019454312 |
| hsa-mir-92-2_r | 0.019454312 |
| HSbrain_1377_l | 0.019454312 |
| HSbrain_1413_l | 0.019454312 |
| HSbrain_321_r | 0.019454312 |
| HSbrain_714_l | 0.019454312 |
| HSbrain_723_l | 0.019454312 |
| HS_291_r | 0.021088597 |
| HS_from_MM_63_l | 0.021088597 |
| HS_from_MQbrain_1260_r | 0.021088597 |
| HS_from_PT_201_r | 0.021088597 |
| HS_from_PT_237_r | 0.021088597 |
| hsa-mir-125a_l | 0.021088597 |
| Hsd_46_l | 0.021088597 |
| Hsd_from_Mmd_527_r | 0.021088597 |
| HS_from_MM_178_r | 0.022745616 |
| HS_from_MM_18_l | 0.022745616 |
| HS_from_MQbrain_207_l | 0.022745616 |
| HS_from_MQbrain_549_l | 0.022745616 |
| hsa-mir-27a_r | 0.022745616 |
| hsa-mir-383_r | 0.022745616 |
| HSbrain_500_r | 0.022745616 |
| HSbrain_597_r | 0.022745616 |
| HSbrain_74_r | 0.022745616 |
| HS_218_r | 0.023736962 |
| HS_from_MM_241_l | 0.023736962 |
| HS_from_MM_319_l | 0.023736962 |
| HS_from_MQbrain_1317_r | 0.023736962 |
| HS_from_MQbrain_1456_l | 0.023736962 |
| HS_from_MQbrain_1632_r | 0.023736962 |
| HS_from_MQbrain_1884_r | 0.023736962 |
| HS_from_PT_114_l | 0.023736962 |
| HS_from_PT_257_r | 0.023736962 |
| HS_from_PT_26_r | 0.023736962 |
| HS_from_PT_32_r | 0.023736962 |
| hsa-mir-29a_r | 0.023736962 |
| HSbrain_243_l | 0.023736962 |
| HSbrain_527_r | 0.023736962 |
| HSbrain_8_r | 0.023736962 |
| Hsd_26_l | 0.023736962 |
| HS_255_l | 0.024762366 |
| HSfrom_MM_334_l | 0.024762366 |
| HS_from_MQbrain_1043_r | 0.024762366 |
| HS_from_MQbrain_1085_l | 0.024762366 |
| HS_from_MQbrain_1300_r | 0.024762366 |
| HS_from_MQbrain_1761_r | 0.024762366 |
| HS_from_MQbrain_1808_r | 0.024762366 |
| HS_from_MQbrain_55_l | 0.024762366 |
| hsa-mir-197_l | 0.024762366 |
| hsa-mir-323_l | 0.024762366 |
| hsa-mir-92-1_r | 0.024762366 |
| HSbrain_1384_r | 0.024762366 |
| HSbrain_362_r | 0.024762366 |
| HSbrain_503_r | 0.024762366 |
| HSbrain_6_r | 0.024762366 |
| HSbrain_778_r | 0.024762366 |
| Hsd_from_Mmd_387_l | 0.024762366 |
| HS_from_MM_121_l | 0.026800941 |
| HS_from_MM_131_l | 0.026800941 |
| HS_from_MQbrain_1684_l | 0.026800941 |
| HS_from_MQbrain_444_r | 0.026800941 |
| HS_from_MQbrain_509_r | 0.026800941 |
| HS_from_MQbrain_734_l | 0.026800941 |
| hsa-mir-598_l | 0.026800941 |
| hsa-mir-647_r | 0.026800941 |
| HSbrain_359_l | 0.026800941 |
| HS_262_r | 0.028655448 |
| HS_from_MQbrain_391_r | 0.028655448 |
| HS_from_MQbrain_74_r | 0.028655448 |
| HS_from_PT_139_l | 0.028655448 |
| hsa-let-7d_l | 0.028655448 |
| hsa-mir-373_r | 0.028655448 |
| hsa-mir-378_r | 0.028655448 |
| hsa-mir-500_l | 0.028655448 |
| HSbrain_318_l | 0.028655448 |
| HSbrain_50_r | 0.028655448 |
| HSbrain_545_r | 0.028655448 |
| Hsd_from_Mmd_302_r | 0.028655448 |
| HS_106_l | 0.02997784 |
| HS_from_MQbrain_1169_l | 0.02997784 |
| HS_from_MQbrain_1832_l | 0.02997784 |
| HS_from_MQbrain_1985_l | 0.02997784 |
| HS_from_MQbrain_918_l | 0.02997784 |
| HS_from_PT_299_l | 0.02997784 |
| hsa-let-7f-2_l | 0.02997784 |
| hsa-mir-134_r | 0.02997784 |
| hsa-mir-146a_l | 0.02997784 |
| hsa-mir-331_r | 0.02997784 |
| hsa-mir-453_l | 0.02997784 |
| hsa-mir-95_r | 0.02997784 |
| HSbrain_114_r | 0.02997784 |
| HSbrain_259_r | 0.02997784 |
| HSbrain_296_l | 0.02997784 |
| HSbrain_546_r | 0.02997784 |
| HSbrain_696_r | 0.02997784 |
| HSbrain_713_l | 0.02997784 |
| HS_118_l | 0.03224042 |
| HS_from_MQbrain_1392_r | 0.03224042 |
| HS_from_MQbrain_1443_r | 0.03224042 |
| HS_from_MQbrain_1445_l | 0.03224042 |
| HS_from_MQbrain_1469_r | 0.03224042 |
| HS_from_MQbrain_1863_l | 0.03224042 |
| HS_from_PT_103_l | 0.03224042 |
| hsa-mir-106b_r | 0.03224042 |
| hsa-mir-145_l | 0.03224042 |
| Hsd_105_l | 0.03224042 |
| Hsd_17_r | 0.03224042 |
| HS_231_r | 0.03374871 |
| HS_from_MM_359_l | 0.03374871 |
| HS_from_MQbrain_1690_l | 0.03374871 |
| HS_from_MQbrain_435_l | 0.03374871 |
| HS_from_MQbrain_482_l | 0.03374871 |
| HS_from_MQbrain_800_r | 0.03374871 |
| HS_from_MQbrain_969_l | 0.03374871 |
| hsa-mir-133a-1_r | 0.03374871 |
| hsa-mir-640_l | 0.03374871 |
| hsa-mir-668_r | 0.03374871 |
| HSbrain_165_l | 0.03374871 |
| HSbrain_316_l | 0.03374871 |
| HSbrain_41_l | 0.03374871 |
| HSbrain_458_l | 0.03374871 |
| HSbrain_500_l | 0.03374871 |
| HSbrain_760_l | 0.03374871 |
| HSbrain_792_r | 0.03374871 |
| Hsd_from_Mmd_487_r | 0.03374871 |
| Hsd_from_Mmd_81_l | 0.03374871 |
| HS_97_r | 0.036099692 |
| HS_from_MQbrain_1340_l | 0.036099692 |
| HS_from_MQbrain_1513_r | 0.036099692 |
| HS_from_PT_295_r | 0.036099692 |
| HS_from_PT_299_r | 0.036099692 |
| hsa-mir-26b_l | 0.036099692 |
| hsa-mir-30c-1_l | 0.036099692 |
| hsa-mir-423_r | 0.036099692 |
| HSbrain_272_l | 0.036099692 |
| Hsd_102_l | 0.036099692 |
| Hsd_from_Mmd_23_l | 0.036099692 |
| Hsd_from_Mmd_410_r | 0.036099692 |
| Hsd_from_Mmd_97_l | 0.036099692 |
| HS_292_l | 0.038273758 |
| HS_33_l | 0.038273758 |
| HS_from_MM_354_r | 0.038273758 |
| HS_from_MQbrain_1087_l | 0.038273758 |
| HS_from_MQbrain_1315_l | 0.038273758 |
| HS_from_MQbrain_1420_l | 0.038273758 |
| HS_from_MQbrain_282_l | 0.038273758 |
| HS_from_MQbrain_498_r | 0.038273758 |
| HS_from_MQbrain_883_l | 0.038273758 |
| hsa-mir-140_l | 0.038273758 |
| hsa-mir-550-2_r | 0.038273758 |
| HSbrain_261_l | 0.038273758 |
| HSbrain_266_r | 0.038273758 |
| HSbrain_543_r | 0.038273758 |
| HSbrain_580_r | 0.038273758 |
| HSbrain_700_r | 0.038273758 |
| HS_189_l | 0.040151024 |
| HS_191_l | 0.040151024 |
| HS_56_r | 0.040151024 |
| HS_from_MM_276_l | 0.040151024 |
| HS_from_MQbrain_1080_l | 0.040151024 |
| HS_from_MQbrain_1379_r | 0.040151024 |
| HS_from_MQbrain_1561_r | 0.040151024 |
| HS_from_MQbrain_1703_r | 0.040151024 |
| HS_from_MQbrain_1747_r | 0.040151024 |
| HS_from_MQbrain_470_l | 0.040151024 |
| HS_from_MQbrain_951_l | 0.040151024 |
| HS_from_PT_43_l | 0.040151024 |
| hsa-let-7a-2_r | 0.040151024 |
| hsa-let-7c_r | 0.040151024 |
| hsa-mir-181a-2_l | 0.040151024 |
| hsa-mir-329-2_r | 0.040151024 |
| HSbrain_109_l | 0.040151024 |
| HSbrain_92_l | 0.040151024 |
| Hsd_from_Mmd_436_r | 0.040151024 |
| Hsd_from_Mmd_527_l | 0.040151024 |
| HS_15_l | 0.042549287 |
| HS_177_l | 0.042549287 |
| HS_261_r | 0.042549287 |
| HS_from_MM_289_l | 0.042549287 |
| HS_from_MQbrain_1227_l | 0.042549287 |
| HS_from_MQbrain_1246_l | 0.042549287 |
| HS_from_MQbrain_1388_l | 0.042549287 |
| HS_from_MQbrain_1844_r | 0.042549287 |
| HS_from_MQbrain_67_r | 0.042549287 |
| HS_from_MQbrain_782_r | 0.042549287 |
| HS_from_PT_63_r | 0.042549287 |
| HS_from_PT_91_r | 0.042549287 |
| hsa-mir-148a_r | 0.042549287 |
| HSbrain_1410_l | 0.042549287 |
| HSbrain_752_r | 0.042549287 |
| Hsd_42_r | 0.042549287 |
| Hsd_from_Mmd_261_l | 0.042549287 |
| HS_266_r | 0.045725655 |
| HS_from_MQbrain_1143_r | 0.045725655 |
| HS_from_MQbrain_1717_r | 0.045725655 |
| HS_from_MQbrain_951_l | 0.045725655 |
| HS_from_PT_21_r | 0.045725655 |
| HS_from_PT_70_r | 0.045725655 |
| hsa-mir-148a_l | 0.045725655 |
| hsa-mir-653_l | 0.045725655 |
| HSbrain_284_l | 0.045725655 |
| Hsd_63_r | 0.045725655 |
| Hsd_from_Mmd_34_r | 0.045725655 |
| Hsd_from_Mmd_422_l | 0.045725655 |
| HS_114_l | 0.048378078 |
| HS_212_l | 0.048378078 |
| HS_from_MM_1_r | 0.048378078 |
| HS_from_MM_32_r | 0.048378078 |
| HS_from_MQbrain_1006_l | 0.048378078 |
| HS_from_MQbrain_1075_l | 0.048378078 |
| HS_from_MQbrain_1752_l | 0.048378078 |
| HS_from_MQbrain_1773_l | 0.048378078 |
| HS_from_MQbrain_472_r | 0.048378078 |
| HS_from_MQbrain_539_r | 0.048378078 |
| HS_from_MQbrain_92_l | 0.048378078 |
| hsa-mir-200c_r | 0.048378078 |
| hsa-mir-92b_r | 0.048378078 |
| HSbrain_1362_l | 0.048378078 |
| HSbrain_25_r | 0.048378078 |
| HSbrain_48_r | 0.048378078 |
| HSbrain_703_l | 0.048378078 |
| Hsd_from_Mmd_49_l | 0.048378078 |
| HS_171_r | 0.050130838 |
| HS_215_l | 0.050130838 |
| HS_93_r | 0.050130838 |
| HS_from_MQbrain_1042_r | 0.050130838 |
| HS_from_MQbrain_1283_l | 0.050130838 |
| HS_from_MQbrain_1610_r | 0.050130838 |
| HS_from_MQbrain_1642_l | 0.050130838 |
| HS_from_MQbrain_1686_l | 0.050130838 |
| HS_from_MQbrain_1771_l | 0.050130838 |
| HS_from_MQbrain_209_r | 0.050130838 |
| HS_from_MQbrain_315_l | 0.050130838 |
| HS_from_MQbrain_343_l | 0.050130838 |
| HS_from_MQbrain_542_r | 0.050130838 |
| HS_from_MQbrain_572_l | 0.050130838 |
| HS_from_MQbrain_691_l | 0.050130838 |
| HS_from_MQbrain_774_r | 0.050130838 |
| HS_from_MQbrain_842_l | 0.050130838 |
| HS_from_MQbrain_901_l | 0.050130838 |
| HS_from_PT_136_l | 0.050130838 |
| hsa-mir-141_r | 0.050130838 |
| hsa-mir-584_r | 0.050130838 |
| hsa-mir-668_l | 0.050130838 |
| hsa-mir-767_r | 0.050130838 |
| HSbrain_585_l | 0.050130838 |
| HSbrain_652_l | 0.050130838 |
| HSbrain_84_l | 0.050130838 |
| Hsd_65_l | 0.050130838 |
| HS_131_r | 0.053159859 |
| HS_from_MQbrain_1239_l | 0.053159859 |
| HS_from_MQbrain_1334_r | 0.053159859 |
| HS_from_MQbrain_1395_r | 0.053159859 |
| HS_from_MQbrain_1484_l | 0.053159859 |
| HS_from_MQbrain_1794_l | 0.053159859 |
| HS_from_MQbrain_1958_l | 0.053159859 |
| HS_from_MQbrain_723_l | 0.053159859 |
| HS_from_MQbrain_873_r | 0.053159859 |
| hsa-mir-520b_r | 0.053159859 |
| HSbrain_374_r | 0.053159859 |
| HSbrain_442_r | 0.053159859 |
| HSbrain_711_l | 0.053159859 |
| Hsd_18_r | 0.053159859 |
| Hsd_37_l | 0.053159859 |
| Hsd_6_l | 0.053159859 |
| Hsd_76_r | 0.053159859 |
| Hsd_from_Mmd_535_r | 0.053159859 |
| HS_59_l | 0.05566524 |
| HS_62_l | 0.05566524 |
| HS_from_MM_115_l | 0.05566524 |
| HS_from_MM_49_l | 0.05566524 |
| HS_from_MM_72_r | 0.05566524 |
| HS_from_MQbrain_1620_l | 0.05566524 |
| HS_from_MQbrain_1674_l | 0.05566524 |
| HS_from_MQbrain_1700_r | 0.05566524 |
| HS_from_MQbrain_38_r | 0.05566524 |
| HS_from_MQbrain_56_l | 0.05566524 |
| HS_from_MQbrain_579_l | 0.05566524 |
| HS_from_MQbrain_664_l | 0.05566524 |
| HS_from_PT_321_r | 0.05566524 |
| hsa-mir-372_r | 0.05566524 |
| hsa-mir-521-1_r | 0.05566524 |
| hsa-mir-652_l | 0.05566524 |
| HSbrain_239_l | 0.05566524 |
| HSbrain_419_l | 0.05566524 |
| HSbrain_671_r | 0.05566524 |
| HSbrain_704_l | 0.05566524 |
| HSbrain_719_l | 0.05566524 |
| HSbrain_85_l | 0.05566524 |
| HSbrain_916_r | 0.05566524 |
| Hsd_from_Mmd_500_r | 0.05566524 |
| HS_109_r | 0.058253679 |
| HS_156_l | 0.058253679 |
| HS_190_l | 0.058253679 |
| HS_from_MQbrain_1099_r | 0.058253679 |
| HS_from_MQbrain_1119_l | 0.058253679 |
| HS_from_MQbrain_1300_l | 0.058253679 |
| HS_from_MQbrain_1374_l | 0.058253679 |
| HS_from_MQbrain_796_l | 0.058253679 |
| HS_from_MQbrain_810_l | 0.058253679 |
| HS_from_MQbrain_883_r | 0.058253679 |
| HS_from_MQbrain_974_l | 0.058253679 |
| HS_from_PT_121_r | 0.058253679 |
| HS_from_PT_187_r | 0.058253679 |
| hsa-mir-148b_r | 0.058253679 |
| hsa-mir-20b_l | 0.058253679 |
| hsa-mir-514-1_r | 0.058253679 |
| hsa-mir-646_r | 0.058253679 |
| HSbrain_123_l | 0.058253679 |
| HSbrain_300_l | 0.058253679 |
| HSbrain_304_r | 0.058253679 |
| HSbrain_35_r | 0.058253679 |
| Hsd_19_r | 0.058253679 |
| Hsd_7_r | 0.058253679 |
| Hsd_from_Mmd_166_l | 0.058253679 |
| Hsd_from_Mmd_70_l | 0.058253679 |
| HS_150_l | 0.061046963 |
| HS_281_r | 0.061046963 |
| HS_304_r | 0.061046963 |
| HS_36_l | 0.061046963 |
| HS_from_MQbrain_128_r | 0.061046963 |
| HS_from_MQbrain_1354_l | 0.061046963 |
| HS_from_MQbrain_1404_r | 0.061046963 |
| HS_from_MQbrain_1412_l | 0.061046963 |
| HS_from_MQbrain_1500_l | 0.061046963 |
| HS_from_MQbrain_1862_l | 0.061046963 |
| HS_from_MQbrain_420_l | 0.061046963 |
| HS_from_MQbrain_43_l | 0.061046963 |
| HS_from_MQbrain_70_l | 0.061046963 |
| HS_from_MQbrain_991_r | 0.061046963 |
| HS_from_PT_242_l | 0.061046963 |
| HS_from_PT_340_l | 0.061046963 |
| hsa-mir-299_r | 0.061046963 |
| hsa-mir-412_r | 0.061046963 |
| hsa-mir-628_r | 0.061046963 |
| HSbrain_214_l | 0.061046963 |
| HSbrain_408_r | 0.061046963 |
| HSbrain_409_r | 0.061046963 |
| HSbrain_663_l | 0.061046963 |
| Hsd_from_Mmd_284_l | 0.061046963 |
| Hsd_from_Mmd_439_l | 0.061046963 |
| HS_180_r | 0.064770288 |
| HS_from_MM_6_r | 0.064770288 |
| HS_from_MQbrain_1235_l | 0.064770288 |
| HS_from_MQbrain_1446_l | 0.064770288 |
| HS_from_MQbrain_1450_r | 0.064770288 |
| HS_from_MQbrain_1696_l | 0.064770288 |
| HS_from_MQbrain_435_r | 0.064770288 |
| HS_from_MQbrain_750_r | 0.064770288 |
| HS_from_PT_308_l | 0.064770288 |
| HS_from_PT_41_r | 0.064770288 |
| hsa-mir-29c_r | 0.064770288 |
| HSbrain_116_l | 0.064770288 |
| HSbrain_1338_r | 0.064770288 |
| HSbrain_157_l | 0.064770288 |
| HSbrain_506_l | 0.064770288 |
| HSbrain_598_l | 0.064770288 |
| HSbrain_755_l | 0.064770288 |
| HSbrain_776_l | 0.064770288 |
| Hsd_81_r | 0.064770288 |
| HS_203_r | 0.067503134 |
| HS_from_MM_342_r | 0.067503134 |
| HS_from_MM_47_l | 0.067503134 |
| HS_from_MQbrain_1164_l | 0.067503134 |
| HS_from_MQbrain_1323_l | 0.067503134 |
| HS_from_MQbrain_1427_l | 0.067503134 |
| HS_from_MQbrain_1877_l | 0.067503134 |
| HS_from_MQbrain_239_r | 0.067503134 |
| HS_from_MQbrain_468_r | 0.067503134 |
| HS_from_MQbrain_549_r | 0.067503134 |
| HS_from_MQbrain_745_r | 0.067503134 |
| HS_from_PT_112_l | 0.067503134 |
| HS_from_PT_339_l | 0.067503134 |
| hsa-mir-137_l | 0.067503134 |
| hsa-mir-329-1_r | 0.067503134 |
| hsa-mir-518f_r | 0.067503134 |
| hsa-mir-524_l | 0.067503134 |
| HSbrain_139_l | 0.067503134 |
| HSbrain_212_l | 0.067503134 |
| HSbrain_330_l | 0.067503134 |
| HSbrain_431_r | 0.067503134 |
| HSbrain_469_l | 0.067503134 |
| HSbrain_572_r | 0.067503134 |
| Hsd_42_l | 0.067503134 |
| Hsd_62_l | 0.067503134 |
| Hsd_from_Mmd_311_l | 0.067503134 |
| Hsd_from_Mmd_370_r | 0.067503134 |
| Hsd_from_Mmd_436_l | 0.067503134 |
| Hsd_from_Mmd_524_r | 0.067503134 |
| HS_from_MM_1_l | 0.069749265 |
| HS_from_MQbrain_1215_l | 0.069749265 |
| HS_from_MQbrain_1376_l | 0.069749265 |
| HS_from_MQbrain_1771_r | 0.069749265 |
| HS_from_MQbrain_1841_r | 0.069749265 |
| HS_from_MQbrain_251_r | 0.069749265 |
| HS_from_MQbrain_29_r | 0.069749265 |
| HS_from_MQbrain_366_r | 0.069749265 |
| HS_from_MQbrain_37_r | 0.069749265 |
| HS_from_MQbrain_641_r | 0.069749265 |
| HS_from_MQbrain_723_r | 0.069749265 |
| HS_from_MQbrain_739_l | 0.069749265 |
| HS_from_MQbrain_997_l | 0.069749265 |
| HS_from_PT_187_l | 0.069749265 |
| HS_from_PT_201_l | 0.069749265 |
| HS_from_PT_235_l | 0.069749265 |
| hsa-mir-124a-3_r | 0.069749265 |
| hsa-mir-193a_l | 0.069749265 |
| hsa-mir-30b_l | 0.069749265 |
| hsa-mir-331_l | 0.069749265 |
| hsa-mir-506_l | 0.069749265 |
| hsa-mir-616_l | 0.069749265 |
| hsa-mir-619_l | 0.069749265 |
| hsa-mir-644_r | 0.069749265 |
| hsa-mir-9-3_r | 0.069749265 |
| HSbrain_155_r | 0.069749265 |
| HSbrain_245_r | 0.069749265 |
| HSbrain_371_r | 0.069749265 |
| HSbrain_610_r | 0.069749265 |
| HSbrain_657_l | 0.069749265 |
| HSbrain_721_r | 0.069749265 |
| HSbrain_728_l | 0.069749265 |
| HSbrain_777_r | 0.069749265 |
| Hsd_50_l | 0.069749265 |
| Hsd_from_Mmd_330_l | 0.069749265 |
| HS_49_r | 0.072349461 |
| HS_81_r | 0.072349461 |
| HS_98_l | 0.072349461 |
| HS_from_MM_289_r | 0.072349461 |
| HS_from_MM_295_r | 0.072349461 |
| HS_from_MQbrain_1082_l | 0.072349461 |
| HS_from_MQbrain_1219_l | 0.072349461 |
| HS_from_MQbrain_1268_l | 0.072349461 |
| HS_from_MQbrain_1280_l | 0.072349461 |
| HS_from_MQbrain_1561_l | 0.072349461 |
| HS_from_MQbrain_1565_r | 0.072349461 |
| HS_from_MQbrain_1658_r | 0.072349461 |
| HS_from_MQbrain_1748_r | 0.072349461 |
| HS_from_MQbrain_253_r | 0.072349461 |
| HS_from_MQbrain_433_l | 0.072349461 |
| HS_from_MQbrain_991_l | 0.072349461 |
| HS_from_PT_170_l | 0.072349461 |
| hsa-mir-199a-2_r | 0.072349461 |
| hsa-mir-200b_r | 0.072349461 |
| hsa-mir-204_r | 0.072349461 |
| hsa-mir-219-1_r | 0.072349461 |
| hsa-mir-224_l | 0.072349461 |
| hsa-mir-363_l | 0.072349461 |
| hsa-mir-449b_r | 0.072349461 |
| HSbrain_1363_l | 0.072349461 |
| HSbrain_1364_l | 0.072349461 |
| HSbrain_250_r | 0.072349461 |
| HSbrain_301_l | 0.072349461 |
| HSbrain_480_r | 0.072349461 |
| HSbrain_507_l | 0.072349461 |
| HSbrain_635_l | 0.072349461 |
| HSbrain_747_l | 0.072349461 |
| HSbrain_749_r | 0.072349461 |
| Hsd_from_Mmd_333_r | 0.072349461 |
| HS_166_r | 0.076797167 |
| HS_66_r | 0.076797167 |
| HS_from_MM_219_l | 0.076797167 |
| HS_from_MM_353_l | 0.076797167 |
| HS_from_MQbrain_1414_r | 0.076797167 |
| HS_from_MQbrain_224_l | 0.076797167 |
| HS_from_MQbrain_590_l | 0.076797167 |
| HS_from_MQbrain_634_l | 0.076797167 |
| HS_from_MQbrain_687_r | 0.076797167 |
| HS_from_MQbrain_698_r | 0.076797167 |
| HS_from_MQbrain_930_r | 0.076797167 |
| HS_from_PT_73_l | 0.076797167 |
| hsa-mir-124a-2_r | 0.076797167 |
| hsa-mir-379_l | 0.076797167 |
| HSbrain_271_l | 0.076797167 |
| HSbrain_344_l | 0.076797167 |
| HSbrain_43_r | 0.076797167 |
| HSbrain_767_r | 0.076797167 |
| HSbrain_788_l | 0.076797167 |
| Hsd_88_r | 0.076797167 |
| HS_119_l | 0.079954597 |
| HS_213_l | 0.079954597 |
| HS_215_r | 0.079954597 |
| HS_223_l | 0.079954597 |
| HS_from_MQbrain_1520_r | 0.079954597 |
| HS_from_MQbrain_1637_r | 0.079954597 |
| HS_from_MQbrain_1665_r | 0.079954597 |
| HS_from_MQbrain_1700_l | 0.079954597 |
| HS_from_MQbrain_1706_l | 0.079954597 |
| HS_from_MQbrain_1745_r | 0.079954597 |
| HS_from_MQbrain_1777_l | 0.079954597 |
| HS_from_MQbrain_576_l | 0.079954597 |
| HS_from_MQbrain_804_r | 0.079954597 |
| HS_from_PT_102_l | 0.079954597 |
| HS_from_PT_148_l | 0.079954597 |
| hsa-mir-124a-1_r | 0.079954597 |
| hsa-mir-199a-1_r | 0.079954597 |
| hsa-mir-641_l | 0.079954597 |
| hsa-mir-9-1_r | 0.079954597 |
| HSbrain_1337_l | 0.079954597 |
| HSbrain_1406_l | 0.079954597 |
| HSbrain_1423_l | 0.079954597 |
| HSbrain_1425_r | 0.079954597 |
| HSbrain_172_l | 0.079954597 |
| HSbrain_317_l | 0.079954597 |
| HSbrain_455_r | 0.079954597 |
| HSbrain_661_l | 0.079954597 |
| HSbrain_797_r | 0.079954597 |
| Hsd_101_l | 0.079954597 |
| Hsd_11_l | 0.079954597 |
| Hsd_33_l | 0.079954597 |
| Hsd_from_Mmd_204_l | 0.079954597 |
| Hsd_from_Mmd_215_r | 0.079954597 |
| HS_57_l | 0.083930001 |
| HS_from_MM_195_r | 0.083930001 |
| HS_from_MM_202_l | 0.083930001 |
| HS_from_MQbrain_1102_l | 0.083930001 |
| HS_from_MQbrain_113_r | 0.083930001 |
| HS_from_MQbrain_1253_r | 0.083930001 |
| HS_from_MQbrain_1325_r | 0.083930001 |
| HS_from_MQbrain_154_l | 0.083930001 |
| HS_from_MQbrain_1593_r | 0.083930001 |
| HS_from_MQbrain_384_r | 0.083930001 |
| HS_from_MQbrain_48_r | 0.083930001 |
| HS_from_MQbrain_597_r | 0.083930001 |
| HS_from_MQbrain_669_r | 0.083930001 |
| HS_from_MQbrain_675_r | 0.083930001 |
| HS_from_MQbrain_715_l | 0.083930001 |
| HS_from_MQbrain_769_r | 0.083930001 |
| HS_from_MQbrain_814_l | 0.083930001 |
| HS_from_PT_20_r | 0.083930001 |
| HS_from_PT_313_r | 0.083930001 |
| hsa-mir-635_l | 0.083930001 |
| HSbrain_286_l | 0.083930001 |
| HSbrain_389_l | 0.083930001 |
| HSbrain_437_r | 0.083930001 |
| HSbrain_463_r | 0.083930001 |
| HSbrain_548_l | 0.083930001 |
| HSbrain_56_l | 0.083930001 |
| HSbrain_711_r | 0.083930001 |
| Hsd_from_Mmd_501_r | 0.083930001 |
| HS_102_l | 0.088143181 |
| HS_11_l | 0.088143181 |
| HS_71_r | 0.088143181 |
| HS_80_l | 0.088143181 |
| HS_from_MM_165_l | 0.088143181 |
| HS_from_MM_229_r | 0.088143181 |
| HS_from_MM_347_l | 0.088143181 |
| HS_from_MQbrain_1064_r | 0.088143181 |
| HS_from_MQbrain_1267_r | 0.088143181 |
| HS_from_MQbrain_1626_r | 0.088143181 |
| HS_from_MQbrain_291_l | 0.088143181 |
| HS_from_MQbrain_686_r | 0.088143181 |
| HS_from_MQbrain_806_l | 0.088143181 |
| HS_from_MQbrain_848_l | 0.088143181 |
| HS_from_MQbrain_948_r | 0.088143181 |
| HS_from_PT_126_l | 0.088143181 |
| HS_from_PT_254_r | 0.088143181 |
| hsa-mir-20a_l | 0.088143181 |
| hsa-mir-375_l | 0.088143181 |
| hsa-mir-521-2_r | 0.088143181 |
| hsa-mir-617_l | 0.088143181 |
| hsa-mir-648_l | 0.088143181 |
| HSbrain_122_l | 0.088143181 |
| HSbrain_124_l | 0.088143181 |
| HSbrain_170_r | 0.088143181 |
| HSbrain_276_l | 0.088143181 |
| HSbrain_530_l | 0.088143181 |
| HSbrain_590_r | 0.088143181 |
| HS_4_r | 0.09152926 |
| HS_from_MM_184_r | 0.09152926 |
| HS_from_MQbrain_1031_l | 0.09152926 |
| HS_from_MQbrain_1037_r | 0.09152926 |
| HS_from_MQbrain_1227_l | 0.09152926 |
| HS_from_MQbrain_1314_l | 0.09152926 |
| HS_from_MQbrain_1417_l | 0.09152926 |
| HS_from_MQbrain_1437_l | 0.09152926 |
| HS_from_MQbrain_1494_l | 0.09152926 |
| HS_from_MQbrain_1592_l | 0.09152926 |
| HS_from_MQbrain_1602_l | 0.09152926 |
| HS_from_MQbrain_1658_l | 0.09152926 |
| HS_from_MQbrain_1678_r | 0.09152926 |
| HS_from_MQbrain_1684_r | 0.09152926 |
| HS_from_MQbrain_1726_l | 0.09152926 |
| HS_from_MQbrain_1758_r | 0.09152926 |
| HS_from_MQbrain_1781_l | 0.09152926 |
| HS_from_MQbrain_1885_r | 0.09152926 |
| HS_from_MQbrain_487_l | 0.09152926 |
| HS_from_MQbrain_550_l | 0.09152926 |
| HS_from_MQbrain_770_r | 0.09152926 |
| HS_from_MQbrain_779_l | 0.09152926 |
| HS_from_PT_95_r | 0.09152926 |
| hsa-let-7f-1_r | 0.09152926 |
| hsa-mir-502_l | 0.09152926 |
| hsa-mir-506_r | 0.09152926 |
| HSbrain_1360_l | 0.09152926 |
| HSbrain_19_l | 0.09152926 |
| HSbrain_237_r | 0.09152926 |
| HSbrain_619_r | 0.09152926 |
| HSbrain_637_l | 0.09152926 |
| HSbrain_642_r | 0.09152926 |
| Hsd_from_Mmd_120_r | 0.09152926 |
| Hsd_from_Mmd_161_l | 0.09152926 |
| Hsd_from_Mmd_371_r | 0.09152926 |
| Hsd_from_Mmd_378_l | 0.09152926 |
| Hsd_from_Mmd_93_r | 0.09152926 |
| HS_129_l | 0.095394629 |
| HS_173_l | 0.095394629 |
| HS_288_l | 0.095394629 |
| HS_61_l | 0.095394629 |
| HS_from_MM_150_r | 0.095394629 |
| HS_from_MQbrain_1201_r | 0.095394629 |
| HS_from_MQbrain_1228_r | 0.095394629 |
| HS_from_MQbrain_1241_r | 0.095394629 |
| HS_from_MQbrain_1545_r | 0.095394629 |
| HS_from_MQbrain_163_l | 0.095394629 |
| HS_from_MQbrain_1648_r | 0.095394629 |
| HS_from_MQbrain_255_l | 0.095394629 |
| HS_from_MQbrain_458_l | 0.095394629 |
| HS_from_MQbrain_755_l | 0.095394629 |
| HS_from_MQbrain_932_l | 0.095394629 |
| HS_from_PT_339_r | 0.095394629 |
| HS_from_PT_50_r | 0.095394629 |
| HS_from_PT_74_r | 0.095394629 |
| hsa-mir-625_r | 0.095394629 |
| HSbrain_113_r | 0.095394629 |
| HSbrain_209_l | 0.095394629 |
| HSbrain_453_r | 0.095394629 |
| HSbrain_473_l | 0.095394629 |
| HSbrain_481_r | 0.095394629 |
| HSbrain_557_r | 0.095394629 |
| HSbrain_570_r | 0.095394629 |
| HSbrain_691_l | 0.095394629 |
| HSbrain_708_l | 0.095394629 |
| HSbrain_717_l | 0.095394629 |
| Hsd_79_l | 0.095394629 |
| Hsd_from_Mmd_166_r | 0.095394629 |
| Hsd_from_Mmd_223_r | 0.095394629 |
| Hsd_from_Mmd_298_l | 0.095394629 |
| Hsd_from_Mmd_324_l | 0.095394629 |
| Hsd_from_Mmd_519_r | 0.095394629 |
| HS_157_l | 0.098921026 |
| HS_306_r | 0.098921026 |
| HS_38_r | 0.098921026 |
| HS_from_MM_103_l | 0.098921026 |
| HS_from_MM_237_l | 0.098921026 |
| HS_from_MM_313_l | 0.098921026 |
| HS_from_MM_34_l | 0.098921026 |
| HS_from_MM_92_r | 0.098921026 |
| HS_from_MQbrain_1139_r | 0.098921026 |
| HS_from_MQbrain_1213_l | 0.098921026 |
| HS_from_MQbrain_1382_l | 0.098921026 |
| HS_from_MQbrain_1410_l | 0.098921026 |
| HS_from_MQbrain_1753_r | 0.098921026 |
| HS_from_MQbrain_2670_r | 0.098921026 |
| HS_from_MQbrain_574_l | 0.098921026 |
| HS_from_MQbrain_868_l | 0.098921026 |
| hsa-let-7c_l | 0.098921026 |
| hsa-mir-30d_l | 0.098921026 |
| hsa-mir-507_l | 0.098921026 |
| hsa-mir-513-1_l | 0.098921026 |
| hsa-mir-518b_r | 0.098921026 |
| hsa-mir-532_l | 0.098921026 |
| hsa-mir-630_r | 0.098921026 |
| hsa-mir-661_r | 0.098921026 |
| HSbrain_1425_l | 0.098921026 |
| HSbrain_240_l | 0.098921026 |
| HSbrain_305_r | 0.098921026 |
| HSbrain_415_l | 0.098921026 |
| HSbrain_540_l | 0.098921026 |
| HSbrain_680_r | 0.098921026 |
| HSbrain_720_l | 0.098921026 |
| HSbrain_722_r | 0.098921026 |
| HSbrain_769_r | 0.098921026 |
| HSbrain_86_r | 0.098921026 |
| Hsd_20_r | 0.098921026 |
| Hsd_from_Mmd_176_l | 0.098921026 |
| Hsd_from_Mmd_318_l | 0.098921026 |
| Hsd_from_Mmd_325_l | 0.098921026 |
| Hsd_from_Mmd_344_l | 0.098921026 |
| Hsd_from_Mmd_480_r | 0.098921026 |
| HS_172_r | 0.103393572 |
| HS_68_l | 0.103393572 |
| HS_from_MM_346_r | 0.103393572 |
| HS_from_MQbrain_1355_r | 0.103393572 |
| HS_from_MQbrain_1442_l | 0.103393572 |
| HS_from_MQbrain_1459_l | 0.103393572 |
| HS_from_MQbrain_1633_r | 0.103393572 |
| HS_from_MQbrain_1744_r | 0.103393572 |
| HS_from_MQbrain_1784_r | 0.103393572 |
| HS_from_MQbrain_412_l | 0.103393572 |
| HS_from_MQbrain_609_l | 0.103393572 |
| HS_from_MQbrain_751_l | 0.103393572 |
| HS_from_MQbrain_882_l | 0.103393572 |
| HS_from_MQbrain_906_r | 0.103393572 |
| HS_from_MQbrain_943_l | 0.103393572 |
| HS_from_PT_22_l | 0.103393572 |
| HS_from_PT_27_l | 0.103393572 |
| hsa-mir-10a_r | 0.103393572 |
| hsa-mir-155_l | 0.103393572 |
| hsa-mir-296_r | 0.103393572 |
| hsa-mir-324_r | 0.103393572 |
| hsa-mir-99a_r | 0.103393572 |
| HSbrain_319_l | 0.103393572 |
| HSbrain_343_l | 0.103393572 |
| HSbrain_482_l | 0.103393572 |
| HSbrain_504_r | 0.103393572 |
| HSbrain_596_r | 0.103393572 |
| HSbrain_673_r | 0.103393572 |
| HSbrain_677_l | 0.103393572 |
| HSbrain_684_r | 0.103393572 |
| HSbrain_94_l | 0.103393572 |
| Hsd_111_r | 0.103393572 |
| Hsd_63_l | 0.103393572 |
| Hsd_from_Mmd_78_r | 0.103393572 |
| HS_101_l | 0.108118292 |
| HS_164_l | 0.108118292 |
| HS_from_MM_124_l | 0.108118292 |
| HS_from_MQbrain_114_r | 0.108118292 |
| HS_from_MQbrain_1366_l | 0.108118292 |
| HS_from_MQbrain_1627_l | 0.108118292 |
| HS_from_MQbrain_1637_l | 0.108118292 |
| HS_from_MQbrain_1840_l | 0.108118292 |
| HS_from_MQbrain_374_r | 0.108118292 |
| HS_from_MQbrain_437_l | 0.108118292 |
| HS_from_MQbrain_546_l | 0.108118292 |
| HS_from_MQbrain_567_r | 0.108118292 |
| HS_from_MQbrain_594_r | 0.108118292 |
| HS_from_MQbrain_80_l | 0.108118292 |
| HS_from_MQbrain_88_l | 0.108118292 |
| HS_from_PT_220_l | 0.108118292 |
| HS_from_PT_7_r | 0.108118292 |
| HS_from_PT_95_l | 0.108118292 |
| hsa-mir-125b-1_r | 0.108118292 |
| hsa-mir-129-1_r | 0.108118292 |
| hsa-mir-185_r | 0.108118292 |
| hsa-mir-211_r | 0.108118292 |
| hsa-mir-483_l | 0.108118292 |
| hsa-mir-526b_r | 0.108118292 |
| HSbrain_280_l | 0.108118292 |
| HSbrain_299_r | 0.108118292 |
| HSbrain_384_l | 0.108118292 |
| HSbrain_506_r | 0.108118292 |
| HSbrain_536_r | 0.108118292 |
| HSbrain_643_l | 0.108118292 |
| HSbrain_691_l | 0.108118292 |
| Hsd_from_Mmd_249_r | 0.108118292 |
| Hsd_from_Mmd_291_r | 0.108118292 |
| Hsd_from_Mmd_478_l | 0.108118292 |
| HS_204_l | 0.113099484 |
| HS_73_r | 0.113099484 |
| HS_98_l | 0.113099484 |
| HS_from_MM_251_r | 0.113099484 |
| HS_from_MM_57_r | 0.113099484 |
| HS_from_MQbrain_105_l | 0.113099484 |
| HS_from_MQbrain_1478_l | 0.113099484 |
| HS_from_MQbrain_1502_l | 0.113099484 |
| HS_from_MQbrain_1695_l | 0.113099484 |
| HS_from_MQbrain_241_l | 0.113099484 |
| HS_from_MQbrain_354_r | 0.113099484 |
| HS_from_MQbrain_604_l | 0.113099484 |
| HS_from_MQbrain_824_l | 0.113099484 |
| HS_from_MQbrain_828_r | 0.113099484 |
| HS_from_MQbrain_921_l | 0.113099484 |
| hsa-mir-224_r | 0.113099484 |
| hsa-mir-377_l | 0.113099484 |
| hsa-mir-571_r | 0.113099484 |
| hsa-mir-770_r | 0.113099484 |
| HSbrain_159_l | 0.113099484 |
| HSbrain_216_l | 0.113099484 |
| HSbrain_227_r | 0.113099484 |
| HSbrain_267_l | 0.113099484 |
| HSbrain_328_l | 0.113099484 |
| HSbrain_390_l | 0.113099484 |
| HSbrain_4_l | 0.113099484 |
| HSbrain_412_l | 0.113099484 |
| HSbrain_47_r | 0.113099484 |
| HSbrain_495_r | 0.113099484 |
| HSbrain_509_l | 0.113099484 |
| HSbrain_520_l | 0.113099484 |
| HSbrain_568_r | 0.113099484 |
| HSbrain_764_r | 0.113099484 |
| Hsd_from_Mmd_391_l | 0.113099484 |
| HS_106_r | 0.11858389 |
| HS_289_l | 0.11858389 |
| HS_from_MQbrain_1051_r | 0.11858389 |
| HS_from_MQbrain_1487_r | 0.11858389 |
| HS_from_MQbrain_1590_l | 0.11858389 |
| HS_from_MQbrain_1750_r | 0.11858389 |
| HS_from_MQbrain_1834_r | 0.11858389 |
| HS_from_MQbrain_1889_l | 0.11858389 |
| HS_from_MQbrain_301_l | 0.11858389 |
| HS_from_MQbrain_502_r | 0.11858389 |
| HS_from_MQbrain_560_r | 0.11858389 |
| HS_from_MQbrain_592_l | 0.11858389 |
| HS_from_MQbrain_897_l | 0.11858389 |
| HS_from_MQbrain_999_l | 0.11858389 |
| hsa-mir-409_r | 0.11858389 |
| hsa-mir-633_r | 0.11858389 |
| hsa-mir-634_l | 0.11858389 |
| HSbrain_1_r | 0.11858389 |
| HSbrain_135_l | 0.11858389 |
| HSbrain_145_l | 0.11858389 |
| HSbrain_220_r | 0.11858389 |
| HSbrain_383_l | 0.11858389 |
| HSbrain_452_l | 0.11858389 |
| HSbrain_664_r | 0.11858389 |
| Hsd_60_r | 0.11858389 |
| Hsd_82_r | 0.11858389 |
| Hsd_from_Mmd_217_r | 0.11858389 |
| Hsd_from_Mmd_311_r | 0.11858389 |
| Hsd_from_Mmd_372_l | 0.11858389 |
| Hsd_from_Mmd_499_r | 0.11858389 |
| Hsd_from_Mmd_518_r | 0.11858389 |
| Hsd_from_Mmd_93_l | 0.11858389 |
| HS_135_l | 0.12422104 |
| HS_25_r | 0.12422104 |
| HS_287_l | 0.12422104 |
| HS_98_r | 0.12422104 |
| HS_from_MM_174_l | 0.12422104 |
| HS_from_MM_298_r | 0.12422104 |
| HS_from_MM_79_l | 0.12422104 |
| HS_from_MM_87_l | 0.12422104 |
| HS_from_MQbrain_1109_l | 0.12422104 |
| HS_from_MQbrain_1184_r | 0.12422104 |
| HS_from_MQbrain_1304_r | 0.12422104 |
| HS_from_MQbrain_1384_r | 0.12422104 |
| HS_from_MQbrain_161_r | 0.12422104 |
| HS_from_MQbrain_406_l | 0.12422104 |
| HS_from_MQbrain_485_l | 0.12422104 |
| HS_from_MQbrain_490_r | 0.12422104 |
| HS_from_MQbrain_789_l | 0.12422104 |
| HS_from_PT_237_l | 0.12422104 |
| HS_from_PT_283_r | 0.12422104 |
| hsa-mir-513-2_l | 0.12422104 |
| hsa-mir-622_l | 0.12422104 |
| HSbrain_1076_l | 0.12422104 |
| HSbrain_131_l | 0.12422104 |
| HSbrain_396_r | 0.12422104 |
| HSbrain_478_r | 0.12422104 |
| HSbrain_50_l | 0.12422104 |
| HSbrain_543_l | 0.12422104 |
| HSbrain_570_l | 0.12422104 |
| Hsd_17_l | 0.12422104 |
| Hsd_from_Mmd_183_r | 0.12422104 |
| Hsd_from_Mmd_237_l | 0.12422104 |
| Hsd_from_Mmd_328_r | 0.12422104 |
| Hsd_from_Mmd_489_r | 0.12422104 |
| HS_109_l | 0.129399391 |
| HS_22_r | 0.129399391 |
| HS_85_l | 0.129399391 |
| HS_from_MM_145_r | 0.129399391 |
| HS_from_MM_157_l | 0.129399391 |
| HS_from_MM_304_r | 0.129399391 |
| HS_from_MQbrain_129_r | 0.129399391 |
| HS_from_MQbrain_137_r | 0.129399391 |
| HS_from_MQbrain_1377_r | 0.129399391 |
| HS_from_MQbrain_1574_l | 0.129399391 |
| HS_from_MQbrain_1712_r | 0.129399391 |
| HS_from_MQbrain_1722_l | 0.129399391 |
| HS_from_MQbrain_1776_l | 0.129399391 |
| HS_from_MQbrain_1794_r | 0.129399391 |
| HS_from_MQbrain_1839_l | 0.129399391 |
| HS_from_MQbrain_36_r | 0.129399391 |
| HS_from_MQbrain_382_l | 0.129399391 |
| HS_from_MQbrain_466_l | 0.129399391 |
| HS_from_MQbrain_555_r | 0.129399391 |
| HS_from_MQbrain_597_l | 0.129399391 |
| HS_from_MQbrain_967_l | 0.129399391 |
| HS_from_PT_207_l | 0.129399391 |
| HS_from_PT_38_l | 0.129399391 |
| hsa-mir-412_l | 0.129399391 |
| hsa-mir-525_l | 0.129399391 |
| hsa-mir-622_r | 0.129399391 |
| hsa-mir-9-2_r | 0.129399391 |
| HSbrain_152_l | 0.129399391 |
| HSbrain_204_r | 0.129399391 |
| HSbrain_244_l | 0.129399391 |
| HSbrain_267_r | 0.129399391 |
| HSbrain_347_r | 0.129399391 |
| HSbrain_448_l | 0.129399391 |
| HSbrain_695_l | 0.129399391 |
| HSbrain_72_l | 0.129399391 |
| HSbrain_970_r | 0.129399391 |
| Hsd_from_Mmd_128_l | 0.129399391 |
| Hsd_from_Mmd_363_l | 0.129399391 |
| Hsd_from_Mmd_431_l | 0.129399391 |
| HS_189_r | 0.134970683 |
| HS_214_r | 0.134970683 |
| HS_261_l | 0.134970683 |
| HS_51_r | 0.134970683 |
| HS_from_MM_119_r | 0.134970683 |
| HS_from_MM_185_r | 0.134970683 |
| HS_from_MQbrain_1081_r | 0.134970683 |
| HS_from_MQbrain_1097_r | 0.134970683 |
| HS_from_MQbrain_1318_l | 0.134970683 |
| HS_from_MQbrain_1376_r | 0.134970683 |
| HS_from_MQbrain_1523_r | 0.134970683 |
| HS_from_MQbrain_1640_l | 0.134970683 |
| HS_from_MQbrain_1692_l | 0.134970683 |
| HS_from_MQbrain_1804_r | 0.134970683 |
| HS_from_MQbrain_1806_l | 0.134970683 |
| HS_from_MQbrain_234_r | 0.134970683 |
| HS_from_MQbrain_461_l | 0.134970683 |
| HS_from_MQbrain_540_l | 0.134970683 |
| HS_from_MQbrain_684_r | 0.134970683 |
| HS_from_MQbrain_89_l | 0.134970683 |
| HS_from_MQbrain_968_l | 0.134970683 |
| HS_from_MQbrain_99_r | 0.134970683 |
| HS_from_PT_194_r | 0.134970683 |
| HS_from_PT_250_r | 0.134970683 |
| hsa-mir-181a-1_r | 0.134970683 |
| hsa-mir-31_r | 0.134970683 |
| hsa-mir-550-1_r | 0.134970683 |
| hsa-mir-588_r | 0.134970683 |
| hsa-mir-609_r | 0.134970683 |
| HSbrain_496_r | 0.134970683 |
| HSbrain_687_r | 0.134970683 |
| HSbrain_715_l | 0.134970683 |
| Hsd_62_l | 0.134970683 |
| Hsd_74_r | 0.134970683 |
| Hsd_from_Mmd_101_r | 0.134970683 |
| Hsd_from_Mmd_291_l | 0.134970683 |
| Hsd_from_Mmd_388_l | 0.134970683 |
| Hsd_from_Mmd_524_l | 0.134970683 |
| HS_111_l | 0.140323812 |
| HS_121_r | 0.140323812 |
| HS_157_r | 0.140323812 |
| HS_161_r | 0.140323812 |
| HS_168_r | 0.140323812 |
| HS_175_l | 0.140323812 |
| HS_219_l | 0.140323812 |
| HS_241_r | 0.140323812 |
| HS_41_r | 0.140323812 |
| HS_53_r | 0.140323812 |
| HS_from_MM_109_l | 0.140323812 |
| HS_from_MM_84_r | 0.140323812 |
| HS_from_MQbrain_1110_l | 0.140323812 |
| HS_from_MQbrain_15_r | 0.140323812 |
| HS_from_MQbrain_1855_l | 0.140323812 |
| HS_from_MQbrain_22_r | 0.140323812 |
| HS_from_MQbrain_221_r | 0.140323812 |
| HS_from_MQbrain_235_r | 0.140323812 |
| HS_from_MQbrain_266_r | 0.140323812 |
| HS_from_MQbrain_467_l | 0.140323812 |
| HS_from_MQbrain_56_r | 0.140323812 |
| HS_from_MQbrain_664_r | 0.140323812 |
| HS_from_PT_170_r | 0.140323812 |
| HS_from_PT_178_l | 0.140323812 |
| HS_from_PT_250_l | 0.140323812 |
| hsa-mir-135a-1_r | 0.140323812 |
| hsa-mir-23a_l | 0.140323812 |
| hsa-mir-26a-1_l | 0.140323812 |
| hsa-mir-27b_r | 0.140323812 |
| hsa-mir-570_l | 0.140323812 |
| hsa-mir-599_r | 0.140323812 |
| HSbrain_108_l | 0.140323812 |
| HSbrain_126_r | 0.140323812 |
| HSbrain_144_r | 0.140323812 |
| HSbrain_361_l | 0.140323812 |
| HSbrain_425_l | 0.140323812 |
| HSbrain_522_r | 0.140323812 |
| HSbrain_541_r | 0.140323812 |
| HSbrain_716_l | 0.140323812 |
| HSbrain_737_r | 0.140323812 |
| Hsd_from_Mmd_123_l | 0.140323812 |
| Hsd_from_Mmd_523_l | 0.140323812 |
| HS_268_l | 0.146822481 |
| HS_76_r | 0.146822481 |
| HS_from_MQbrain_1030_l | 0.146822481 |
| HS_from_MQbrain_1036_r | 0.146822481 |
| HS_from_MQbrain_1222_r | 0.146822481 |
| HS_from_MQbrain_1250_r | 0.146822481 |
| HS_from_MQbrain_1381_r | 0.146822481 |
| HS_from_MQbrain_1462_r | 0.146822481 |
| HS_from_MQbrain_1493_r | 0.146822481 |
| HS_from_MQbrain_1675_r | 0.146822481 |
| HS_from_MQbrain_1695_r | 0.146822481 |
| HS_from_MQbrain_1759_l | 0.146822481 |
| HS_from_MQbrain_1807_l | 0.146822481 |
| HS_from_MQbrain_260_r | 0.146822481 |
| HS_from_MQbrain_283_r | 0.146822481 |
| HS_from_MQbrain_600_l | 0.146822481 |
| HS_from_MQbrain_67_l | 0.146822481 |
| HS_from_MQbrain_697_l | 0.146822481 |
| HS_from_MQbrain_97_r | 0.146822481 |
| HS_from_PT_219_l | 0.146822481 |
| HS_from_PT_271_l | 0.146822481 |
| HS_from_PT_284_r | 0.146822481 |
| hsa-mir-1-1_l | 0.146822481 |
| hsa-mir-521-1_l | 0.146822481 |
| HSbrain_107_r | 0.146822481 |
| HSbrain_1298_r | 0.146822481 |
| HSbrain_130_l | 0.146822481 |
| HSbrain_139_r | 0.146822481 |
| HSbrain_599_r | 0.146822481 |
| HSbrain_624_l | 0.146822481 |
| HSbrain_663_r | 0.146822481 |
| HSbrain_705_r | 0.146822481 |
| Hsd_77_r | 0.146822481 |
| Hsd_96_r | 0.146822481 |
| Hsd_from_Mmd_316_r | 0.146822481 |
| HS_115_r | 0.152858542 |
| HS_176_l | 0.152858542 |
| HS_from_MM_89_l | 0.152858542 |
| HS_from_MQbrain_1072_r | 0.152858542 |
| HS_from_MQbrain_1127_r | 0.152858542 |
| HS_from_MQbrain_153_l | 0.152858542 |
| HS_from_MQbrain_154_r | 0.152858542 |
| HS_from_MQbrain_1817_l | 0.152858542 |
| HS_from_MQbrain_1896_r | 0.152858542 |
| HS_from_MQbrain_338_l | 0.152858542 |
| HS_from_MQbrain_340_l | 0.152858542 |
| HS_from_MQbrain_355_r | 0.152858542 |
| HS_from_MQbrain_6_r | 0.152858542 |
| HS_from_MQbrain_689_l | 0.152858542 |
| HS_from_MQbrain_693_r | 0.152858542 |
| HS_from_MQbrain_707_l | 0.152858542 |
| HS_from_MQbrain_969_r | 0.152858542 |
| HS_from_PT_238_l | 0.152858542 |
| HS_from_PT_92_l | 0.152858542 |
| hsa-mir-17_l | 0.152858542 |
| hsa-mir-188_l | 0.152858542 |
| hsa-mir-641_r | 0.152858542 |
| HSbrain_130_r | 0.152858542 |
| HSbrain_1325_r | 0.152858542 |
| HSbrain_171_l | 0.152858542 |
| HSbrain_22_l | 0.152858542 |
| HSbrain_222_r | 0.152858542 |
| HSbrain_266_l | 0.152858542 |
| HSbrain_306_r | 0.152858542 |
| HSbrain_32_r | 0.152858542 |
| HSbrain_483_r | 0.152858542 |
| HSbrain_604_r | 0.152858542 |
| HSbrain_641_r | 0.152858542 |
| HSbrain_656_l | 0.152858542 |
| HSbrain_695_r | 0.152858542 |
| HSbrain_717_r | 0.152858542 |
| HSbrain_752_l | 0.152858542 |
| HSbrain_811_l | 0.152858542 |
| Hsd_38_r | 0.152858542 |
| Hsd_from_Mmd_280_l | 0.152858542 |
| Hsd_from_Mmd_346_l | 0.152858542 |
| HS_128_l | 0.158799149 |
| HS_18_l | 0.158799149 |
| HS_95_r | 0.158799149 |
| HS_from_MM_118_l | 0.158799149 |
| HS_from_MM_135_l | 0.158799149 |
| HS_from_MM_35_l | 0.158799149 |
| HS_from_MQbrain_1018_l | 0.158799149 |
| HS_from_MQbrain_1204_r | 0.158799149 |
| HS_from_MQbrain_140_l | 0.158799149 |
| HS_from_MQbrain_1501_r | 0.158799149 |
| HS_from_MQbrain_155_l | 0.158799149 |
| HS_from_MQbrain_1831_r | 0.158799149 |
| HS_from_MQbrain_2699_l | 0.158799149 |
| HS_from_MQbrain_281_r | 0.158799149 |
| HS_from_MQbrain_382_r | 0.158799149 |
| HS_from_MQbrain_414_l | 0.158799149 |
| HS_from_MQbrain_6_l | 0.158799149 |
| HS_from_MQbrain_625_l | 0.158799149 |
| HS_from_MQbrain_631_l | 0.158799149 |
| HS_from_MQbrain_675_l | 0.158799149 |
| HS_from_MQbrain_68_r | 0.158799149 |
| HS_from_MQbrain_900_r | 0.158799149 |
| HS_from_MQbrain_912_l | 0.158799149 |
| HS_from_PT_158_l | 0.158799149 |
| HS_from_PT_255_l | 0.158799149 |
| hsa-mir-151_l | 0.158799149 |
| hsa-mir-16-2_l | 0.158799149 |
| hsa-mir-422a_l | 0.158799149 |
| hsa-mir-518c_l | 0.158799149 |
| hsa-mir-98_r | 0.158799149 |
| HSbrain_121_l | 0.158799149 |
| HSbrain_248_l | 0.158799149 |
| HSbrain_320_l | 0.158799149 |
| HSbrain_347_l | 0.158799149 |
| HSbrain_413_l | 0.158799149 |
| HSbrain_55_l | 0.158799149 |
| HSbrain_653_l | 0.158799149 |
| HSbrain_669_r | 0.158799149 |
| HSbrain_673_l | 0.158799149 |
| HSbrain_768_l | 0.158799149 |
| HSbrain_783_l | 0.158799149 |
| Hsd_67_r | 0.158799149 |
| Hsd_from_Mmd_334_r | 0.158799149 |
| Hsd_from_Mmd_44_r | 0.158799149 |
| HS_181_l | 0.163891418 |
| HS_286_r | 0.163891418 |
| HS_74_r | 0.163891418 |
| HS_from_MM_218_l | 0.163891418 |
| HS_from_MM_340_r | 0.163891418 |
| HS_from_MQbrain_1028_l | 0.163891418 |
| HS_from_MQbrain_1090_r | 0.163891418 |
| HS_from_MQbrain_1119_l | 0.163891418 |
| HS_from_MQbrain_1212_l | 0.163891418 |
| HS_from_MQbrain_1223_l | 0.163891418 |
| HS_from_MQbrain_1426_l | 0.163891418 |
| HS_from_MQbrain_1520_l | 0.163891418 |
| HS_from_MQbrain_1535_r | 0.163891418 |
| HS_from_MQbrain_1721_l | 0.163891418 |
| HS_from_MQbrain_1724_l | 0.163891418 |
| HS_from_MQbrain_365_r | 0.163891418 |
| HS_from_MQbrain_449_l | 0.163891418 |
| HS_from_MQbrain_529_r | 0.163891418 |
| HS_from_MQbrain_566_r | 0.163891418 |
| HS_from_MQbrain_596_r | 0.163891418 |
| HS_from_MQbrain_629_r | 0.163891418 |
| HS_from_MQbrain_650_r | 0.163891418 |
| HS_from_MQbrain_824_r | 0.163891418 |
| HS_from_MQbrain_936_r | 0.163891418 |
| HS_from_MQbrain_941_r | 0.163891418 |
| HS_from_MQbrain_996_l | 0.163891418 |
| HS_from_PT_155_l | 0.163891418 |
| HS_from_PT_196_r | 0.163891418 |
| HS_from_PT_234_l | 0.163891418 |
| HS_from_PT_280_l | 0.163891418 |
| hsa-mir-124a-1_l | 0.163891418 |
| hsa-mir-30c-2_l | 0.163891418 |
| hsa-mir-33b_l | 0.163891418 |
| hsa-mir-652_r | 0.163891418 |
| HSbrain_275_r | 0.163891418 |
| HSbrain_304_l | 0.163891418 |
| HSbrain_305_l | 0.163891418 |
| HSbrain_376_l | 0.163891418 |
| HSbrain_402_l | 0.163891418 |
| HSbrain_438_r | 0.163891418 |
| HSbrain_446_r | 0.163891418 |
| HSbrain_578_l | 0.163891418 |
| HSbrain_598_r | 0.163891418 |
| HSbrain_680_l | 0.163891418 |
| HSbrain_784_r | 0.163891418 |
| HSbrain_804_l | 0.163891418 |
| HSbrain_95_l | 0.163891418 |
| Hsd_108_r | 0.163891418 |
| Hsd_84_r | 0.163891418 |
| Hsd_from_Mmd_19_l | 0.163891418 |
| Hsd_from_Mmd_455_l | 0.163891418 |
| Hsd_from_Mmd_52_r | 0.163891418 |
| Hsd_from_Mmd_94_l | 0.163891418 |
| HS_128_r | 0.170271053 |
| HS_151_r | 0.170271053 |
| HS_192_r | 0.170271053 |
| HS_197_r | 0.170271053 |
| HS_212_r | 0.170271053 |
| HS_223_r | 0.170271053 |
| HS_264_r | 0.170271053 |
| HS_78_r | 0.170271053 |
| HS_83_r | 0.170271053 |
| HS_from_MM_163_l | 0.170271053 |
| HS_from_MM_232_r | 0.170271053 |
| HS_from_MM_304_l | 0.170271053 |
| HS_from_MQbrain_1199_l | 0.170271053 |
| HS_from_MQbrain_1377_l | 0.170271053 |
| HS_from_MQbrain_1846_r | 0.170271053 |
| HS_from_MQbrain_1893_r | 0.170271053 |
| HS_from_MQbrain_281_l | 0.170271053 |
| HS_from_MQbrain_392_r | 0.170271053 |
| HS_from_MQbrain_468_l | 0.170271053 |
| HS_from_MQbrain_603_r | 0.170271053 |
| HS_from_MQbrain_841_l | 0.170271053 |
| HS_from_MQbrain_97_l | 0.170271053 |
| hsa-mir-192_r | 0.170271053 |
| hsa-mir-196b_r | 0.170271053 |
| hsa-mir-765_r | 0.170271053 |
| HSbrain_143_l | 0.170271053 |
| HSbrain_177_l | 0.170271053 |
| HSbrain_216_r | 0.170271053 |
| HSbrain_364_r | 0.170271053 |
| HSbrain_41_r | 0.170271053 |
| HSbrain_467_l | 0.170271053 |
| HSbrain_53_l | 0.170271053 |
| HSbrain_701_l | 0.170271053 |
| HSbrain_724_l | 0.170271053 |
| HSbrain_726_l | 0.170271053 |
| HSbrain_745_l | 0.170271053 |
| HSbrain_755_r | 0.170271053 |
| HSbrain_780_l | 0.170271053 |
| HSbrain_80_l | 0.170271053 |
| Hsd_47_r | 0.170271053 |
| Hsd_from_Mmd_235_r | 0.170271053 |
| Hsd_from_Mmd_306_l | 0.170271053 |
| Hsd_from_Mmd_35_l | 0.170271053 |
| Hsd_from_Mmd_367_l | 0.170271053 |
| Hsd_from_Mmd_410_l | 0.170271053 |
| HS_130_l | 0.176183859 |
| HS_145_r | 0.176183859 |
| HS_186_r | 0.176183859 |
| HS_204_r | 0.176183859 |
| HS_220_l | 0.176183859 |
| HS_from_MM_123_l | 0.176183859 |
| HS_from_MM_84_l | 0.176183859 |
| HS_from_MQbrain_1126_l | 0.176183859 |
| HS_from_MQbrain_1204_l | 0.176183859 |
| HS_from_MQbrain_1245_l | 0.176183859 |
| HS_from_MQbrain_135_l | 0.176183859 |
| HS_from_MQbrain_1444_l | 0.176183859 |
| HS_from_MQbrain_1497_l | 0.176183859 |
| HS_from_MQbrain_1565_l | 0.176183859 |
| HS_from_MQbrain_1566_r | 0.176183859 |
| HS_from_MQbrain_1773_l | 0.176183859 |
| HS_from_MQbrain_1832_r | 0.176183859 |
| HS_from_MQbrain_2087_r | 0.176183859 |
| HS_from_MQbrain_314_l | 0.176183859 |
| HS_from_MQbrain_365_l | 0.176183859 |
| HS_from_MQbrain_520_l | 0.176183859 |
| HS_from_MQbrain_628_l | 0.176183859 |
| HS_from_MQbrain_855_r | 0.176183859 |
| HS_from_MQbrain_984_l | 0.176183859 |
| HS_from_PT_27_r | 0.176183859 |
| hsa-mir-101-1_r | 0.176183859 |
| hsa-mir-144_r | 0.176183859 |
| hsa-mir-16-1_l | 0.176183859 |
| hsa-mir-432_l | 0.176183859 |
| hsa-mir-526a-1_r | 0.176183859 |
| hsa-mir-92-1_l | 0.176183859 |
| HSbrain_120_r | 0.176183859 |
| HSbrain_1418_r | 0.176183859 |
| HSbrain_151_r | 0.176183859 |
| HSbrain_32_l | 0.176183859 |
| HSbrain_334_r | 0.176183859 |
| HSbrain_491_l | 0.176183859 |
| HSbrain_590_l | 0.176183859 |
| HSbrain_626_l | 0.176183859 |
| HSbrain_707_r | 0.176183859 |
| HSbrain_771_l | 0.176183859 |
| Hsd_27_r | 0.176183859 |
| Hsd_65_r | 0.176183859 |
| Hsd_from_Mmd_123_r | 0.176183859 |
| Hsd_from_Mmd_212_r | 0.176183859 |
| Hsd_from_Mmd_296_l | 0.176183859 |
| Hsd_from_Mmd_328_l | 0.176183859 |
| Hsd_from_Mmd_344_r | 0.176183859 |
| Hsd_from_Mmd_391_r | 0.176183859 |
| Hsd_from_Mmd_412_r | 0.176183859 |
| Hsd_from_Mmd_92_l | 0.176183859 |
| HS_132_l | 0.182758186 |
| HS_167_l | 0.182758186 |
| HS_307_r | 0.182758186 |
| HS_from_MM_124_r | 0.182758186 |
| HS_from_MM_149_r | 0.182758186 |
| HS_from_MM_160_l | 0.182758186 |
| HS_from_MM_342_l | 0.182758186 |
| HS_from_MM_6_l | 0.182758186 |
| HS_from_MQbrain_1137_l | 0.182758186 |
| HS_from_MQbrain_1219_r | 0.182758186 |
| HS_from_MQbrain_1349_l | 0.182758186 |
| HS_from_MQbrain_1380_r | 0.182758186 |
| HS_from_MQbrain_147_l | 0.182758186 |
| HS_from_MQbrain_1509_r | 0.182758186 |
| HS_from_MQbrain_1576_r | 0.182758186 |
| HS_from_MQbrain_162_r | 0.182758186 |
| HS_from_MQbrain_1793_l | 0.182758186 |
| HS_from_MQbrain_1883_l | 0.182758186 |
| HS_from_MQbrain_361_l | 0.182758186 |
| HS_from_MQbrain_559_r | 0.182758186 |
| HS_from_MQbrain_635_l | 0.182758186 |
| HS_from_MQbrain_676_r | 0.182758186 |
| HS_from_MQbrain_694_r | 0.182758186 |
| HS_from_MQbrain_732_r | 0.182758186 |
| HS_from_MQbrain_736_r | 0.182758186 |
| HS_from_MQbrain_850_l | 0.182758186 |
| HS_from_MQbrain_881_l | 0.182758186 |
| HS_from_PT_104_l | 0.182758186 |
| HS_from_PT_161_l | 0.182758186 |
| HS_from_PT_188_l | 0.182758186 |
| hsa-mir-1-2_l | 0.182758186 |
| hsa-mir-190_r | 0.182758186 |
| hsa-mir-26a-2_l | 0.182758186 |
| hsa-mir-383_l | 0.182758186 |
| hsa-mir-432_r | 0.182758186 |
| hsa-mir-514-3_r | 0.182758186 |
| hsa-mir-520f_r | 0.182758186 |
| hsa-mir-624_l | 0.182758186 |
| hsa-mir-93_l | 0.182758186 |
| HSbrain_388_r | 0.182758186 |
| HSbrain_456_r | 0.182758186 |
| HSbrain_706_l | 0.182758186 |
| Hsd_from_Mmd_187_r | 0.182758186 |
| Hsd_from_Mmd_190_r | 0.182758186 |
| Hsd_from_Mmd_208_r | 0.182758186 |
| Hsd_from_Mmd_232_r | 0.182758186 |
| Hsd_from_Mmd_333_l | 0.182758186 |
| Hsd_from_Mmd_68_l | 0.182758186 |
| HS_190_r | 0.188867139 |
| HS_77_l | 0.188867139 |
| HS_from_MM_182_r | 0.188867139 |
| HS_from_MM_298_l | 0.188867139 |
| HS_from_MM_331_r | 0.188867139 |
| HS_from_MM_48_l | 0.188867139 |
| HS_from_MM_86_l | 0.188867139 |
| HS_from_MQbrain_105_l | 0.188867139 |
| HS_from_MQbrain_1073_r | 0.188867139 |
| HS_from_MQbrain_120_l | 0.188867139 |
| HS_from_MQbrain_1208_l | 0.188867139 |
| HS_from_MQbrain_1224_r | 0.188867139 |
| HS_from_MQbrain_1234_l | 0.188867139 |
| HS_from_MQbrain_1353_l | 0.188867139 |
| HS_from_MQbrain_1596_r | 0.188867139 |
| HS_from_MQbrain_1734_l | 0.188867139 |
| HS_from_MQbrain_1760_r | 0.188867139 |
| HS_from_MQbrain_178_r | 0.188867139 |
| HS_from_MQbrain_436_r | 0.188867139 |
| HS_from_MQbrain_487_r | 0.188867139 |
| HS_from_MQbrain_524_l | 0.188867139 |
| HS_from_MQbrain_65_l | 0.188867139 |
| HS_from_MQbrain_768_l | 0.188867139 |
| HS_from_MQbrain_791_r | 0.188867139 |
| HS_from_MQbrain_999_r | 0.188867139 |
| HS_from_PT_22_l | 0.188867139 |
| HS_from_PT_247_l | 0.188867139 |
| HS_from_PT_271_r | 0.188867139 |
| HS_from_PT_3_l | 0.188867139 |
| HS_from_PT_328_l | 0.188867139 |
| HS_from_PT_99_r | 0.188867139 |
| hsa-mir-183_r | 0.188867139 |
| hsa-mir-203_r | 0.188867139 |
| hsa-mir-21_l | 0.188867139 |
| hsa-mir-587_r | 0.188867139 |
| hsa-mir-604_l | 0.188867139 |
| HSbrain_153_r | 0.188867139 |
| HSbrain_156_l | 0.188867139 |
| HSbrain_198_l | 0.188867139 |
| HSbrain_210_l | 0.188867139 |
| HSbrain_242_l | 0.188867139 |
| HSbrain_270_l | 0.188867139 |
| HSbrain_370_r | 0.188867139 |
| HSbrain_403_l | 0.188867139 |
| HSbrain_471_r | 0.188867139 |
| HSbrain_553_l | 0.188867139 |
| HSbrain_571_l | 0.188867139 |
| HSbrain_627_r | 0.188867139 |
| Hsd_from_Mmd_120_l | 0.188867139 |
| Hsd_from_Mmd_180_r | 0.188867139 |
| Hsd_from_Mmd_253_l | 0.188867139 |
| Hsd_from_Mmd_274_r | 0.188867139 |
| Hsd_from_Mmd_314_r | 0.188867139 |
| Hsd_from_Mmd_325_r | 0.188867139 |
| HS_124_l | 0.19639408 |
| HS_198_l | 0.19639408 |
| HS_203_l | 0.19639408 |
| HS_265_l | 0.19639408 |
| HS_77_r | 0.19639408 |
| HS_from_MM_194_l | 0.19639408 |
| HS_from_MQbrain_1402_r | 0.19639408 |
| HS_from_MQbrain_1424_r | 0.19639408 |
| HS_from_MQbrain_1671_l | 0.19639408 |
| HS_from_MQbrain_1783_r | 0.19639408 |
| HS_from_MQbrain_188_l | 0.19639408 |
| HS_from_MQbrain_1883_r | 0.19639408 |
| HS_from_MQbrain_383_l | 0.19639408 |
| HS_from_MQbrain_389_l | 0.19639408 |
| HS_from_MQbrain_406_r | 0.19639408 |
| HS_from_MQbrain_648_l | 0.19639408 |
| HS_from_MQbrain_84_r | 0.19639408 |
| HS_from_MQbrain_901_r | 0.19639408 |
| HS_from_PT_130_r | 0.19639408 |
| HS_from_PT_196_l | 0.19639408 |
| HS_from_PT_204_r | 0.19639408 |
| HS_from_PT_48_l | 0.19639408 |
| hsa-mir-139_l | 0.19639408 |
| hsa-mir-324_l | 0.19639408 |
| hsa-mir-454_l | 0.19639408 |
| hsa-mir-662_l | 0.19639408 |
| HSbrain_143_r | 0.19639408 |
| HSbrain_180_l | 0.19639408 |
| HSbrain_237_l | 0.19639408 |
| HSbrain_295_r | 0.19639408 |
| HSbrain_325_r | 0.19639408 |
| HSbrain_433_r | 0.19639408 |
| HSbrain_476_r | 0.19639408 |
| HSbrain_615_l | 0.19639408 |
| HSbrain_616_r | 0.19639408 |
| HSbrain_658_l | 0.19639408 |
| HSbrain_690_r | 0.19639408 |
| HSbrain_70_r | 0.19639408 |
| HSbrain_810_l | 0.19639408 |
| Hsd_19_l | 0.19639408 |
| Hsd_25_l | 0.19639408 |
| Hsd_from_Mmd_197_l | 0.19639408 |
| Hsd_from_Mmd_259_r | 0.19639408 |
| Hsd_from_Mmd_326_l | 0.19639408 |
| Hsd_from_Mmd_399_r | 0.19639408 |

¹Wilcoxon analysis: cut off adj. p value <0.2

TABLE 2b
Up- or downregulated small RNAs adenoma compared to control

| | | | | adenomas/ |
|---|---|---|---|---|
| | adeno- | adenoma | controls | controls |
| | ma vs | (avg | (avg | (ratio |
| | control | expres- | expres- | expres- |
| Systematic Name | (adj. p)¹ | sion) | sion) | sion)² |
| HS_from_MQbrain_485_l | 0.124 | 0.621 | 1.997 | −1.377 |
| HSbrain_412_l | 0.113 | 0.278 | 1.614 | −1.336 |
| HS_from_MQbrain_1497_l | 0.176 | 1.843 | 3.166 | −1.323 |
| HS_from_MQbrain_594_r | 0.108 | 0.859 | 2.180 | −1.321 |
| HS_from_MQbrain_1109_l | 0.124 | 0.527 | 1.813 | −1.286 |
| HS_from_MQbrain_967_l | 0.129 | 0.867 | 2.144 | −1.277 |
| HS_from_MQbrain_137_r | 0.129 | 0.859 | 2.113 | −1.254 |
| HS_from_MQbrain_1082_l | 0.072 | −1.727 | −0.502 | −1.225 |
| HS_from_PT_155_l | 0.164 | 1.326 | 2.524 | −1.198 |
| HS_from_MQbrain_1334_r | 0.053 | −1.612 | −0.419 | −1.192 |
| HSbrain_724_l | 0.170 | −1.686 | −0.535 | −1.151 |
| HS_from_MQbrain_340_l | 0.153 | 0.732 | 1.879 | −1.146 |
| HSbrain_126_r | 0.140 | 0.598 | 1.713 | −1.115 |
| HSbrain_653_l | 0.159 | 0.620 | 1.725 | −1.105 |
| HS_from_PT_104_l | 0.183 | 0.615 | 1.720 | −1.105 |
| HS_from_MQbrain_1353_l | 0.189 | −1.557 | −0.453 | −1.104 |
| hsa-mir-124a-3_r | 0.070 | 0.106 | 1.207 | −1.101 |
| HS_from_MQbrain_1793_l | 0.183 | 0.546 | 1.638 | −1.093 |
| hsa-mir-124a-2_r | 0.077 | 0.186 | 1.276 | −1.090 |
| HS_38_r | 0.099 | −1.398 | −0.321 | −1.077 |
| HS_from_MQbrain_855_r | 0.176 | 1.019 | 2.091 | −1.072 |
| HS_from_MQbrain_634_l | 0.077 | −1.384 | −0.322 | −1.063 |
| hsa-mir-124a-1_r | 0.080 | 0.236 | 1.287 | −1.051 |
| HS_from_MQbrain_1832_r | 0.176 | −1.661 | −0.631 | −1.030 |
| HS_from_MQbrain_932_l | 0.095 | −1.329 | −0.301 | −1.028 |
| HS_from_MQbrain_1424_r | 0.196 | −1.461 | −0.448 | −1.013 |
| HS_from_MQbrain_281_r | 0.159 | −1.396 | −0.386 | −1.010 |
| Hsd_from_Mmd_94_l | 0.164 | −0.053 | 0.946 | −0.999 |
| HS_from_MQbrain_825_r | 0.012 | −0.915 | 0.083 | −0.998 |
| HS_261_r | 0.043 | −1.240 | −0.249 | −0.992 |
| HS_from_MQbrain_436_r | 0.189 | −1.739 | −0.758 | −0.981 |
| HSbrain_491_l | 0.176 | −1.399 | −0.425 | −0.974 |
| HS_from_PT_95_l | 0.108 | −1.290 | −0.327 | −0.963 |
| HS_from_MM_163_l | 0.170 | −1.554 | −0.612 | −0.942 |
| HS_from_MQbrain_1268_l | 0.072 | −1.227 | −0.286 | −0.941 |
| HS_203_l | 0.196 | 0.658 | 1.592 | −0.934 |
| HSbrain_680_r | 0.099 | −1.254 | −0.338 | −0.916 |
| HS_from_MM_123_l | 0.176 | −1.163 | −0.251 | −0.912 |
| HS_from_MQbrain_635_l | 0.183 | −1.339 | −0.431 | −0.908 |
| HS_from_MQbrain_824_l | 0.113 | −1.224 | −0.329 | −0.895 |
| HS_from_PT_130_r | 0.196 | 0.515 | 1.410 | −0.895 |
| HS_71_l | 0.004 | −0.861 | 0.033 | −0.893 |
| HS_from_MQbrain_37_r | 0.070 | −1.191 | −0.299 | −0.892 |
| Hsd_101_l | 0.080 | −1.212 | −0.322 | −0.889 |
| HS_51_r | 0.135 | −1.387 | −0.499 | −0.888 |
| Hsd_from_Mmd_326_l | 0.196 | −1.132 | −0.252 | −0.881 |
| HS_from_MQbrain_968_l | 0.135 | −1.191 | −0.319 | −0.872 |
| HSbrain_673_r | 0.103 | −1.174 | −0.303 | −0.871 |
| HS_97_r | 0.036 | 0.155 | 1.026 | −0.871 |
| HSbrain_1406_l | 0.080 | −0.299 | 0.569 | −0.868 |
| HS_262_l | 0.001 | −0.228 | 0.636 | −0.864 |
| HS_203_r | 0.068 | 0.078 | 0.937 | −0.859 |
| HSbrain_458_r | 0.020 | −0.452 | 0.407 | −0.858 |
| HSbrain_687_r | 0.135 | −1.259 | −0.403 | −0.855 |
| Hsd_from_Mmd_412_r | 0.176 | −1.668 | −0.814 | −0.855 |
| HSbrain_270_l | 0.189 | 0.524 | 1.376 | −0.852 |
| hsa-mir-449b_r | 0.072 | −0.894 | −0.053 | −0.841 |
| HSbrain_684_r | 0.103 | −1.235 | −0.412 | −0.822 |
| HS_from_MQbrain_991_l | 0.072 | 0.438 | 1.259 | −0.821 |
| HS_from_PT_283_r | 0.124 | −1.162 | −0.351 | −0.812 |
| HSbrain_695_l | 0.129 | −1.139 | −0.329 | −0.810 |
| HS_106_r | 0.119 | −1.141 | −0.333 | −0.808 |
| HS_from_MM_169_r | 0.004 | −0.743 | 0.062 | −0.804 |
| HS_from_MM_353_l | 0.077 | 0.019 | 0.823 | −0.804 |
| HS_from_MQbrain_1502_l | 0.113 | −1.097 | −0.293 | −0.804 |
| HSbrain_198_l | 0.189 | −1.309 | −0.506 | −0.803 |
| HSbrain_711_r | 0.084 | 0.379 | 1.174 | −0.794 |
| HSbrain_570_r | 0.095 | −1.044 | −0.258 | −0.786 |
| hsa-mir-635_l | 0.084 | 0.520 | 1.301 | −0.781 |
| HS_from_MQbrain_1561_r | 0.040 | −0.679 | 0.100 | −0.779 |
| HS_from_PT_36_l | 0.013 | −0.741 | 0.038 | −0.778 |
| hsa-mir-524_l | 0.068 | −0.064 | 0.713 | −0.778 |
| Hsd_from_Mmd_372_l | 0.119 | −0.938 | −0.161 | −0.776 |
| HSbrain_669_r | 0.159 | −1.197 | −0.432 | −0.764 |
| HS_from_MQbrain_1520_l | 0.164 | −1.097 | −0.337 | −0.760 |
| HS_from_MQbrain_736_r | 0.183 | −1.132 | −0.374 | −0.758 |
| HS_128_l | 0.159 | −0.990 | −0.238 | −0.752 |
| HS_from_MQbrain_1085_l | 0.005 | −0.935 | −0.185 | −0.751 |
| Hsd_from_Mmd_452_l | 0.025 | −0.286 | 0.462 | −0.748 |
| HSbrain_656_l | 0.153 | 0.468 | 1.216 | −0.747 |
| HSbrain_296_l | 0.030 | 0.085 | 0.828 | −0.742 |
| HSbrain_389_l | 0.084 | −0.915 | −0.173 | −0.742 |
| HS_from_MM_118_r | 0.004 | −0.556 | 0.179 | −0.735 |
| Hsd_from_Mmd_328_l | 0.176 | 0.566 | 1.299 | −0.733 |
| HSbrain_157_l | 0.065 | −0.837 | −0.111 | −0.726 |
| HS_from_MQbrain_559_r | 0.183 | −1.064 | −0.346 | −0.718 |
| HS_from_MQbrain_48_r | 0.084 | −0.848 | −0.132 | −0.715 |
| HSbrain_41_r | 0.170 | −0.877 | −0.162 | −0.715 |
| hsa-mir-185_r | 0.108 | 0.201 | 0.911 | −0.710 |
| HS_from_MQbrain_15_r | 0.140 | −1.076 | −0.367 | −0.709 |
| HS_from_MQbrain_301_l | 0.119 | −1.043 | −0.336 | −0.706 |
| HSbrain_453_r | 0.095 | −0.857 | −0.156 | −0.701 |
| Hsd_105_l | 0.032 | −0.841 | −0.141 | −0.700 |
| hsa-mir-591_r | 0.017 | 0.025 | 0.724 | −0.699 |
| HSbrain_47_r | 0.113 | −0.986 | −0.288 | −0.699 |
| HS_from_PT_207_l | 0.129 | −0.921 | −0.223 | −0.697 |
| HS_from_MQbrain_1087_l | 0.038 | −0.740 | −0.043 | −0.697 |
| HS_from_MM_118_l | 0.159 | −0.915 | −0.220 | −0.695 |
| HS_from_MQbrain_161_r | 0.124 | 0.226 | 0.920 | −0.694 |
| HS_from_MQbrain_354_r | 0.113 | −0.882 | −0.189 | −0.692 |
| HS_261_l | 0.135 | 0.324 | 1.016 | −0.692 |
| HS_from_MQbrain_1648_r | 0.095 | 0.036 | 0.726 | −0.691 |
| HS_from_MQbrain_1246_l | 0.043 | 0.389 | 1.078 | −0.689 |
| HSbrain_616_r | 0.196 | 0.375 | 1.064 | −0.688 |
| HS_98_l | 0.072 | −1.008 | −0.320 | −0.688 |
| HSbrain_304_r | 0.058 | −0.783 | −0.097 | −0.687 |
| HS_from_MQbrain_314_l | 0.176 | −0.924 | −0.243 | −0.681 |
| HSbrain_583_l | 0.021 | −0.586 | 0.095 | −0.681 |
| HS_from_MQbrain_629_r | 0.164 | −0.976 | −0.296 | −0.680 |
| Hsd_from_Mmd_485_r | 0.025 | −0.553 | 0.126 | −0.679 |
| HS_from_PT_3_l | 0.189 | 0.352 | 1.026 | −0.675 |
| HS_from_MQbrain_1500_l | 0.061 | 0.178 | 0.850 | −0.672 |
| HS_175_l | 0.140 | −0.909 | −0.238 | −0.671 |
| HS_from_MQbrain_1219_r | 0.183 | −0.953 | −0.285 | −0.668 |
| HSbrain_172_l | 0.080 | 0.083 | 0.747 | −0.664 |
| Hsd_from_Mmd_117_r | 0.025 | −0.532 | 0.129 | −0.661 |
| hsa-mir-383_r | 0.016 | −0.034 | 0.626 | −0.660 |
| Hsd_107_l | 0.024 | −0.573 | 0.086 | −0.659 |
| HS_145_r | 0.176 | −1.153 | −0.497 | −0.657 |
| HSbrain_690_r | 0.196 | −0.957 | −0.301 | −0.657 |
| HSbrain_2_l | 0.019 | −0.562 | 0.092 | −0.654 |
| HS_181_l | 0.164 | −1.057 | −0.406 | −0.651 |
| HS_176_l | 0.153 | −0.933 | −0.282 | −0.651 |
| HS_from_MQbrain_1099_r | 0.058 | −0.662 | −0.013 | −0.649 |
| HS_9_r | 0.004 | −0.731 | −0.083 | −0.649 |
| hsa-mir-619_l | 0.070 | −0.336 | 0.309 | −0.645 |
| HS_from_MQbrain_694_r | 0.183 | 0.350 | 0.995 | −0.645 |
| HS_from_MQbrain_1081_r | 0.135 | −0.031 | 0.613 | −0.644 |
| HS_241_r | 0.140 | 0.465 | 1.104 | −0.639 |
| HS_from_MQbrain_458_l | 0.095 | −0.816 | −0.178 | −0.638 |
| HSbrain_704_l | 0.056 | 0.198 | 0.830 | −0.632 |
| HS_from_MQbrain_1444_l | 0.008 | −1.059 | −0.429 | −0.630 |
| HSbrain_159_l | 0.113 | −1.013 | −0.385 | −0.628 |
| HS_from_MM_135_l | 0.159 | −0.023 | 0.604 | −0.627 |
| HSbrain_344_l | 0.077 | −0.642 | −0.017 | −0.624 |
| HS_from_MQbrain_520_l | 0.176 | −0.948 | −0.325 | −0.623 |
| HSbrain_390_l | 0.113 | −0.817 | −0.194 | −0.622 |
| HSbrain_657_l | 0.070 | −0.850 | −0.228 | −0.622 |
| HS_from_MQbrain_901_r | 0.196 | 0.174 | 0.796 | −0.622 |
| HSbrain_456_r | 0.183 | −0.828 | −0.207 | −0.621 |
| HSbrain_120_r | 0.176 | 0.508 | 1.125 | −0.617 |
| HS_from_PT_295_r | 0.036 | −0.497 | 0.120 | −0.617 |
| HS_from_MQbrain_1535_r | 0.164 | −0.833 | −0.216 | −0.617 |
| HSbrain_695_r | 0.153 | 0.176 | 0.791 | −0.615 |
| HSbrain_719_l | 0.056 | 0.061 | 0.674 | −0.613 |
| HSbrain_305_l | 0.164 | −1.071 | −0.461 | −0.610 |
| HS_from_MQbrain_1773_l | 0.048 | −0.942 | −0.335 | −0.607 |
| HSbrain_571_l | 0.189 | −0.891 | −0.291 | −0.600 |
| HSbrain_216_l | 0.113 | −0.857 | −0.258 | −0.599 |
| HSbrain_107_r | 0.147 | −0.715 | −0.116 | −0.599 |
| hsa-mir-661_r | 0.099 | −0.380 | 0.217 | −0.597 |
| HSbrain_122_l | 0.088 | −0.799 | −0.204 | −0.595 |
| HS_132_l | 0.183 | −0.675 | −0.082 | −0.593 |
| HSbrain_325_r | 0.196 | −1.053 | −0.460 | −0.592 |
| HS_157_r | 0.140 | −0.878 | −0.286 | −0.592 |
| HS_from_MQbrain_560_r | 0.119 | −0.731 | −0.139 | −0.592 |
| HS_212_r | 0.170 | −0.314 | 0.278 | −0.591 |
| HS_from_PT_284_r | 0.147 | 0.474 | 1.062 | −0.589 |
| HS_from_PT_103_l | 0.032 | −0.507 | 0.080 | −0.587 |
| HS_from_MQbrain_1042_r | 0.050 | −0.400 | 0.186 | −0.586 |
| HS_from_PT_41_r | 0.065 | −0.324 | 0.261 | −0.585 |
| HS_from_MQbrain_178_r | 0.189 | −0.684 | −0.099 | −0.584 |
| Hsd_from_Mmd_120_l | 0.189 | −0.867 | −0.285 | −0.583 |
| HS_from_MQbrain_1442_l | 0.103 | 0.047 | 0.630 | −0.583 |
| HSbrain_707_r | 0.176 | −0.556 | 0.021 | −0.577 |
| Hsd_from_Mmd_296_l | 0.176 | −0.110 | 0.466 | −0.576 |
| HS_from_MQbrain_235_r | 0.140 | −0.811 | −0.237 | −0.575 |
| Hsd_from_Mmd_346_l | 0.153 | −0.928 | −0.355 | −0.573 |
| HS_from_PT_187_l | 0.070 | −0.656 | −0.086 | −0.570 |
| HS_204_r | 0.176 | −0.741 | −0.172 | −0.569 |
| HSbrain_177_l | 0.170 | −0.752 | −0.183 | −0.568 |
| HS_173_l | 0.095 | −0.578 | −0.009 | −0.568 |
| HS_from_MQbrain_1314_l | 0.092 | −0.577 | −0.009 | −0.568 |
| HS_from_MQbrain_755_l | 0.095 | 0.177 | 0.741 | −0.564 |
| HS_from_MQbrain_1753_r | 0.099 | 0.239 | 0.803 | −0.564 |
| HS_from_MQbrain_1376_r | 0.135 | 0.327 | 0.890 | −0.563 |
| HSbrain_530_l | 0.088 | −0.657 | −0.097 | −0.560 |
| HSbrain_153_r | 0.189 | −0.773 | −0.214 | −0.559 |
| HS_from_MQbrain_1478_l | 0.113 | −0.834 | −0.275 | −0.559 |
| Hsd_67_r | 0.159 | −0.803 | −0.245 | −0.558 |
| HS_from_MQbrain_129_r | 0.129 | −0.659 | −0.101 | −0.557 |
| Hsd_from_Mmd_370_r | 0.068 | 0.017 | 0.572 | −0.555 |
| Hsd_from_Mmd_473_l | 0.025 | −0.251 | 0.301 | −0.553 |
| Hsd_42_l | 0.068 | −0.650 | −0.100 | −0.550 |
| HS_from_MM_276_l | 0.040 | −0.253 | 0.297 | −0.550 |
| HS_from_MM_39_l | 0.005 | −0.155 | 0.394 | −0.549 |
| HS_from_MQbrain_574_l | 0.099 | 0.327 | 0.875 | −0.548 |
| HSbrain_777_r | 0.070 | −0.877 | −0.332 | −0.545 |
| HSbrain_598_r | 0.164 | 0.144 | 0.689 | −0.545 |
| HSbrain_726_l | 0.170 | −0.919 | −0.375 | −0.544 |
| HS_98_l | 0.072 | −0.947 | −0.406 | −0.541 |
| HS_from_MM_218_l | 0.164 | −0.955 | −0.415 | −0.540 |
| HS_from_PT_201_l | 0.070 | −0.573 | −0.034 | −0.539 |
| HS_from_MQbrain_1006_l | 0.048 | −0.593 | −0.055 | −0.538 |
| HSbrain_22_l | 0.153 | −0.159 | 0.378 | −0.537 |
| HS_from_MQbrain_1204_l | 0.176 | −0.915 | −0.381 | −0.534 |
| HS_from_MQbrain_65_l | 0.189 | −0.083 | 0.451 | −0.534 |
| HS_from_MQbrain_1633_r | 0.103 | −0.635 | −0.102 | −0.533 |
| HS_from_MQbrain_1783_r | 0.196 | 0.348 | 0.880 | −0.532 |
| HS_from_MQbrain_1402_r | 0.196 | −0.824 | −0.294 | −0.530 |
| HS_from_MQbrain_1883_l | 0.183 | −0.608 | −0.081 | −0.527 |
| HS_231_r | 0.034 | −0.796 | −0.271 | −0.525 |
| HSbrain_713_r | 0.023 | −0.564 | −0.041 | −0.523 |
| HSbrain_578_l | 0.164 | −0.619 | −0.099 | −0.520 |
| HSbrain_442_r | 0.053 | 0.220 | 0.740 | −0.519 |
| HS_from_MQbrain_437_l | 0.108 | −0.563 | −0.049 | −0.514 |
| HS_from_MQbrain_1626_r | 0.088 | 0.001 | 0.509 | −0.509 |
| Hsd_from_Mmd_324_l | 0.095 | −0.734 | −0.227 | −0.508 |
| Hsd_from_Mmd_523_l | 0.140 | 0.149 | 0.654 | −0.505 |
| HSbrain_703_r | 0.023 | −0.500 | 0.004 | −0.504 |
| HS_from_MQbrain_843_r | 0.012 | −0.512 | −0.013 | −0.499 |
| HSbrain_347_r | 0.129 | −0.638 | −0.143 | −0.495 |
| HS_from_MQbrain_1444_l | 0.008 | −0.527 | −0.034 | −0.494 |
| HSbrain_1423_l | 0.080 | −0.583 | −0.092 | −0.491 |
| HSbrain_343_l | 0.103 | −0.579 | −0.093 | −0.486 |
| HSbrain_86_r | 0.099 | −0.605 | −0.122 | −0.483 |
| HS_from_MQbrain_1877_l | 0.068 | −0.227 | 0.255 | −0.482 |
| HS_from_MQbrain_1103_l | 0.005 | −0.190 | 0.290 | −0.480 |
| HS_from_MQbrain_1893_r | 0.170 | 0.202 | 0.682 | −0.480 |
| HSbrain_463_r | 0.084 | −0.222 | 0.257 | −0.480 |
| HSbrain_752_r | 0.043 | −0.613 | −0.135 | −0.478 |
| HS_from_MQbrain_1377_r | 0.129 | 0.283 | 0.761 | −0.477 |
| HSbrain_747_l | 0.072 | −0.034 | 0.441 | −0.475 |
| HS_62_l | 0.056 | −0.564 | −0.091 | −0.473 |
| HS_from_MM_131_l | 0.027 | −0.261 | 0.210 | −0.471 |
| hsa-mir-640_l | 0.034 | −0.148 | 0.322 | −0.470 |
| HS_from_MQbrain_487_l | 0.092 | −0.841 | −0.371 | −0.470 |
| HS_from_MQbrain_590_l | 0.077 | −0.280 | 0.189 | −0.469 |
| HS_135_l | 0.124 | −0.144 | 0.323 | −0.467 |
| hsa-mir-196a-2_r | 0.015 | −0.123 | 0.342 | −0.465 |
| HS_from_MQbrain_597_r | 0.084 | −0.606 | −0.142 | −0.464 |
| HS_from_MQbrain_697_l | 0.147 | −0.518 | −0.057 | −0.462 |
| HS_from_MQbrain_365_l | 0.176 | 0.235 | 0.696 | −0.461 |
| Hsd_from_Mmd_212_r | 0.176 | −0.510 | −0.051 | −0.459 |
| HSbrain_446_l | 0.020 | −0.397 | 0.062 | −0.459 |
| HSbrain_32_r | 0.153 | −0.616 | −0.158 | −0.458 |
| HSbrain_483_r | 0.153 | 0.145 | 0.602 | −0.456 |
| HSbrain_334_r | 0.176 | −0.377 | 0.076 | −0.452 |
| HS_from_MQbrain_1137_l | 0.183 | −0.675 | −0.223 | −0.452 |
| Hsd_27_r | 0.176 | 0.166 | 0.616 | −0.450 |
| HS_from_MQbrain_113_r | 0.084 | 0.052 | 0.502 | −0.450 |
| hsa-mir-324_r | 0.103 | −0.112 | 0.337 | −0.449 |
| HSbrain_304_l | 0.164 | 0.017 | 0.465 | −0.448 |
| HSbrain_629_l | 0.023 | −0.646 | −0.199 | −0.447 |
| Hsd_from_Mmd_306_l | 0.170 | −0.590 | −0.145 | −0.445 |
| hsa-mir-372_r | 0.056 | 0.185 | 0.630 | −0.444 |
| HS_from_MQbrain_1317_r | 0.007 | −0.193 | 0.251 | −0.444 |
| hsa-mir-525_l | 0.129 | 0.196 | 0.638 | −0.442 |
| HS_from_MQbrain_1806_l | 0.135 | −0.081 | 0.357 | −0.438 |
| HS_from_MM_298_r | 0.124 | −0.087 | 0.350 | −0.437 |
| hsa-mir-644_r | 0.070 | −0.262 | 0.173 | −0.435 |
| HS_from_MQbrain_814_l | 0.084 | 0.092 | 0.526 | −0.435 |
| HS_from_MQbrain_906_r | 0.103 | −0.642 | −0.208 | −0.434 |
| Hsd_from_Mmd_487_r | 0.034 | −0.529 | −0.096 | −0.433 |
| HS_from_MQbrain_1208_l | 0.189 | 0.334 | 0.765 | −0.431 |
| Hsd_from_Mmd_311_r | 0.119 | −0.006 | 0.423 | −0.429 |
| HS_from_MQbrain_1771_l | 0.050 | −0.654 | −0.226 | −0.429 |
| HS_171_r | 0.050 | −0.274 | 0.154 | −0.428 |
| HS_from_MQbrain_1752_l | 0.048 | −0.014 | 0.411 | −0.425 |
| HS_from_MQbrain_1840_l | 0.108 | −0.025 | 0.399 | −0.424 |
| HSbrain_437_r | 0.084 | −0.052 | 0.373 | −0.424 |
| HS_from_MQbrain_1245_l | 0.176 | −0.711 | −0.287 | −0.424 |
| HS_71_r | 0.088 | −0.153 | 0.270 | −0.423 |
| Hsd_88_r | 0.077 | −0.265 | 0.157 | −0.422 |
| HS_from_MQbrain_1437_l | 0.092 | −0.244 | 0.177 | −0.421 |
| HSbrain_755_l | 0.065 | −0.427 | −0.006 | −0.421 |
| HS_from_MQbrain_1817_l | 0.153 | 0.002 | 0.421 | −0.420 |
| HSbrain_151_r | 0.176 | −0.522 | −0.103 | −0.419 |
| HS_from_MQbrain_567_r | 0.108 | −0.378 | 0.039 | −0.417 |
| HSbrain_155_r | 0.070 | −0.057 | 0.360 | −0.417 |
| HSbrain_43_r | 0.077 | −0.371 | 0.046 | −0.417 |
| hsa-mir-584_r | 0.050 | −0.203 | 0.213 | −0.416 |
| HS_from_PT_91_r | 0.043 | −0.068 | 0.345 | −0.413 |
| HS_from_PT_126_l | 0.088 | −0.052 | 0.358 | −0.411 |
| Hsd_from_Mmd_399_r | 0.196 | −0.367 | 0.042 | −0.410 |
| HS_from_MQbrain_1420_l | 0.038 | −0.388 | 0.019 | −0.407 |
| HSbrain_12_l | 0.017 | −0.292 | 0.112 | −0.404 |
| HSbrain_764_r | 0.113 | −0.045 | 0.357 | −0.402 |
| HS_from_MQbrain_1070_r | 0.005 | −0.290 | 0.110 | −0.400 |
| HS_223_l | 0.080 | −0.213 | 0.186 | −0.399 |
| HS_from_MQbrain_163_l | 0.095 | −0.645 | −0.248 | −0.397 |
| HS_from_MQbrain_1119_l | 0.058 | −0.111 | 0.285 | −0.397 |
| HS_from_MQbrain_1748_r | 0.072 | −0.171 | 0.224 | −0.394 |
| HSbrain_319_l | 0.103 | −0.232 | 0.161 | −0.393 |
| HSbrain_116_l | 0.065 | −0.732 | −0.339 | −0.393 |
| Hsd_65_l | 0.050 | −0.128 | 0.263 | −0.390 |
| HSbrain_1325_r | 0.153 | −0.451 | −0.061 | −0.390 |
| HSbrain_755_r | 0.170 | −0.635 | −0.246 | −0.389 |
| HSbrain_753_r | 0.024 | −0.232 | 0.156 | −0.388 |
| HS_from_PT_242_l | 0.061 | −0.402 | −0.017 | −0.386 |
| HSbrain_48_r | 0.048 | −0.110 | 0.274 | −0.384 |
| HS_93_r | 0.050 | −0.412 | −0.030 | −0.382 |
| Hsd_from_Mmd_120_r | 0.092 | −0.425 | −0.048 | −0.377 |
| HS_from_MQbrain_1494_l | 0.092 | 0.310 | 0.687 | −0.376 |
| HSbrain_481_r | 0.095 | −0.825 | −0.449 | −0.376 |
| Hsd_63_l | 0.103 | −0.283 | 0.092 | −0.375 |
| HS_from_MM_219_l | 0.077 | −0.105 | 0.265 | −0.370 |
| HS_from_MQbrain_912_l | 0.159 | 0.449 | 0.817 | −0.369 |
| HS_from_MQbrain_1073_r | 0.189 | −0.122 | 0.246 | −0.368 |
| HS_81_r | 0.072 | −0.099 | 0.269 | −0.367 |
| HS_from_MQbrain_842_l | 0.050 | 0.049 | 0.415 | −0.366 |
| HS_from_PT_321_r | 0.056 | −0.070 | 0.295 | −0.365 |
| HSbrain_320_l | 0.159 | 0.052 | 0.414 | −0.362 |
| HS_from_MQbrain_524_l | 0.189 | −0.475 | −0.117 | −0.358 |
| HSbrain_135_l | 0.119 | −0.146 | 0.211 | −0.358 |
| HS_from_MQbrain_991_r | 0.061 | −0.017 | 0.337 | −0.354 |
| HS_from_PT_170_r | 0.140 | −0.438 | −0.084 | −0.354 |
| HS_from_MQbrain_1051_r | 0.119 | 0.156 | 0.509 | −0.353 |
| HS_from_MQbrain_391_r | 0.029 | −0.169 | 0.183 | −0.352 |
| HS_from_MQbrain_140_l | 0.159 | 0.077 | 0.427 | −0.350 |
| HSbrain_438_r | 0.164 | −0.066 | 0.282 | −0.348 |
| HS_from_MQbrain_804_r | 0.080 | −0.287 | 0.061 | −0.348 |
| Hsd_from_Mmd_391_l | 0.113 | −0.103 | 0.245 | −0.348 |
| HSbrain_476_r | 0.196 | 0.063 | 0.410 | −0.347 |
| HS_from_MQbrain_745_r | 0.068 | −0.292 | 0.053 | −0.345 |
| HS_from_MQbrain_1102_l | 0.084 | −0.516 | −0.173 | −0.343 |
| HS_from_MQbrain_868_l | 0.099 | −0.413 | −0.071 | −0.342 |
| HS_from_MQbrain_984_l | 0.176 | −0.423 | −0.082 | −0.341 |
| HS_from_MQbrain_1792_r | 0.010 | −0.296 | 0.045 | −0.341 |
| HS_from_MQbrain_603_r | 0.170 | −0.178 | 0.161 | −0.339 |
| HS_from_MQbrain_365_r | 0.164 | −0.252 | 0.085 | −0.337 |
| HS_from_MQbrain_1075_l | 0.048 | −0.648 | −0.312 | −0.336 |
| HS_172_r | 0.103 | 0.080 | 0.416 | −0.336 |
| HSbrain_384_l | 0.108 | −0.164 | 0.172 | −0.336 |
| HS_from_MQbrain_406_l | 0.124 | −0.273 | 0.063 | −0.336 |
| HS_from_PT_339_l | 0.068 | −0.719 | −0.383 | −0.336 |
| Hsd_from_Mmd_535_r | 0.053 | −0.453 | −0.118 | −0.335 |
| Hsd_84_r | 0.164 | −0.195 | 0.136 | −0.332 |
| HS_from_MQbrain_1565_l | 0.176 | −0.209 | 0.123 | −0.332 |
| HS_from_MQbrain_420_l | 0.061 | −0.349 | −0.020 | −0.329 |
| HS_from_PT_201_r | 0.013 | −0.178 | 0.150 | −0.328 |
| HS_265_l | 0.196 | 0.045 | 0.372 | −0.327 |
| HS_from_MQbrain_1726_l | 0.092 | −0.112 | 0.214 | −0.327 |
| hsa-mir-197_l | 0.016 | −0.405 | −0.080 | −0.325 |
| HS_from_MQbrain_114_r | 0.108 | −0.679 | −0.354 | −0.325 |
| HS_from_MQbrain_355_r | 0.153 | 0.155 | 0.479 | −0.324 |
| HSbrain_703_l | 0.048 | −0.443 | −0.121 | −0.323 |
| Hsd_from_Mmd_155_l | 0.025 | −0.274 | 0.048 | −0.322 |
| HS_from_MQbrain_435_r | 0.065 | −0.103 | 0.219 | −0.322 |
| HS_from_MQbrain_1043_r | 0.005 | −0.624 | −0.302 | −0.322 |
| HSbrain_1298_r | 0.147 | −0.271 | 0.050 | −0.321 |
| HS_from_MQbrain_1690_l | 0.034 | −0.631 | −0.313 | −0.318 |
| HS_from_PT_250_l | 0.140 | 0.222 | 0.539 | −0.318 |
| hsa-mir-377_l | 0.113 | −0.086 | 0.229 | −0.316 |
| HS_from_MQbrain_686_r | 0.088 | −0.636 | −0.322 | −0.314 |
| HSbrain_691_l | 0.095 | −0.865 | −0.552 | −0.313 |
| HSbrain_370_r | 0.189 | −0.088 | 0.225 | −0.313 |
| HS_from_MQbrain_1484_l | 0.053 | −0.038 | 0.271 | −0.308 |
| HS_from_MQbrain_89_l | 0.135 | −0.128 | 0.179 | −0.307 |
| HS_from_MQbrain_361_l | 0.183 | 0.178 | 0.484 | −0.306 |
| HS_from_MQbrain_596_r | 0.164 | −0.114 | 0.192 | −0.306 |
| HS_from_MQbrain_600_l | 0.147 | −0.210 | 0.096 | −0.306 |
| HS_from_PT_92_l | 0.153 | −0.695 | −0.390 | −0.305 |
| HS_from_MQbrain_1624_l | 0.009 | −0.245 | 0.058 | −0.303 |
| HS_from_PT_73_l | 0.077 | 0.058 | 0.359 | −0.301 |
| HS_from_MQbrain_1374_l | 0.058 | −0.430 | −0.130 | −0.299 |
| Hsd_49_r | 0.025 | −0.276 | 0.023 | −0.299 |
| HS_from_MQbrain_1596_r | 0.189 | −0.280 | 0.018 | −0.298 |
| HS_from_MM_174_l | 0.124 | −0.325 | −0.027 | −0.298 |
| hsa-mir-204_r | 0.072 | −0.384 | −0.087 | −0.297 |
| HS_from_MQbrain_1426_l | 0.164 | −0.698 | −0.404 | −0.293 |
| HS_220_l | 0.176 | −0.135 | 0.158 | −0.293 |
| HS_from_MQbrain_1839_l | 0.129 | −0.692 | −0.400 | −0.292 |
| HS_from_MM_346_r | 0.103 | −0.463 | −0.173 | −0.290 |
| HS_from_MQbrain_1119_l | 0.058 | −0.615 | −0.325 | −0.290 |
| HSbrain_1425_l | 0.099 | −0.622 | −0.332 | −0.290 |
| Hsd_from_Mmd_363_l | 0.129 | −0.293 | −0.003 | −0.289 |
| HS_from_MQbrain_162_r | 0.183 | −0.461 | −0.172 | −0.289 |
| HS_198_l | 0.196 | −0.603 | −0.314 | −0.289 |
| HS_from_PT_188_l | 0.183 | −0.268 | 0.020 | −0.288 |
| Hsd_50_l | 0.070 | −0.532 | −0.244 | −0.288 |
| HSbrain_143_r | 0.196 | −0.381 | −0.093 | −0.287 |
| HSbrain_663_l | 0.061 | −0.005 | 0.282 | −0.287 |
| HSbrain_109_l | 0.040 | −0.582 | −0.296 | −0.286 |
| HS_from_MQbrain_382_l | 0.129 | −0.075 | 0.210 | −0.285 |
| HS_from_MQbrain_675_l | 0.159 | −0.628 | −0.343 | −0.285 |
| HS_from_MQbrain_1671_l | 0.196 | −0.314 | −0.030 | −0.284 |
| HS_from_MQbrain_999_r | 0.189 | −0.845 | −0.563 | −0.283 |
| hsa-mir-148a_l | 0.046 | −0.199 | 0.082 | −0.281 |
| HS_from_MQbrain_974_l | 0.058 | −0.613 | −0.333 | −0.280 |
| HS_from_MQbrain_1627_l | 0.108 | −0.614 | −0.335 | −0.280 |
| HS_from_MM_359_l | 0.034 | −0.086 | 0.193 | −0.279 |
| HSbrain_25_r | 0.048 | −0.147 | 0.132 | −0.279 |
| HSbrain_568_r | 0.113 | −0.156 | 0.122 | −0.278 |
| HS_49_r | 0.072 | 0.167 | 0.445 | −0.278 |
| HSbrain_316_l | 0.034 | −0.303 | −0.025 | −0.278 |
| Hsd_108_r | 0.164 | −0.266 | 0.012 | −0.278 |
| HS_from_MQbrain_1678_r | 0.092 | −0.080 | 0.198 | −0.277 |
| HS_from_MQbrain_943_l | 0.103 | −0.647 | −0.370 | −0.277 |
| HS_167_l | 0.183 | −0.391 | −0.114 | −0.277 |
| HS_from_MQbrain_1566_r | 0.176 | −0.302 | −0.026 | −0.277 |
| HS_from_MQbrain_466_l | 0.129 | −0.308 | −0.034 | −0.275 |
| HS_from_MQbrain_99_r | 0.135 | −0.627 | −0.352 | −0.275 |
| HS_from_PT_271_r | 0.189 | −0.271 | 0.002 | −0.273 |
| HSbrain_642_r | 0.092 | 0.356 | 0.627 | −0.271 |
| HS_from_MQbrain_433_l | 0.072 | −0.495 | −0.225 | −0.270 |
| hsa-mir-622_l | 0.124 | −0.337 | −0.068 | −0.269 |
| HSbrain_717_l | 0.095 | −0.453 | −0.184 | −0.269 |
| HS_from_PT_170_l | 0.072 | −0.362 | −0.094 | −0.269 |
| HSbrain_85_l | 0.056 | 0.065 | 0.333 | −0.268 |
| hsa-mir-483_l | 0.108 | −0.570 | −0.302 | −0.267 |
| HS_from_PT_112_l | 0.068 | −0.217 | 0.050 | −0.267 |
| HS_from_MQbrain_1758_r | 0.092 | −0.494 | −0.229 | −0.264 |
| HSbrain_139_l | 0.068 | −0.533 | −0.269 | −0.264 |
| HSbrain_32_l | 0.176 | −0.350 | −0.087 | −0.263 |
| HS_98_r | 0.124 | −0.635 | −0.373 | −0.261 |
| HS_from_MM_342_l | 0.183 | 0.142 | 0.402 | −0.260 |
| HSbrain_284_l | 0.046 | −0.589 | −0.331 | −0.258 |
| HS_157_l | 0.099 | −0.493 | −0.236 | −0.257 |
| HS_from_MQbrain_1777_l | 0.080 | −0.385 | −0.131 | −0.253 |
| Hsd_from_Mmd_284_l | 0.061 | −0.006 | 0.246 | −0.253 |
| HS_102_l | 0.088 | −0.528 | −0.276 | −0.252 |
| HS_from_MQbrain_1127_r | 0.153 | 0.133 | 0.384 | −0.251 |
| HS_from_MQbrain_1620_l | 0.056 | −0.368 | −0.118 | −0.250 |
| HS_from_MQbrain_734_l | 0.027 | −0.281 | −0.031 | −0.250 |
| HSbrain_664_r | 0.119 | −0.538 | −0.288 | −0.250 |
| HS_101_l | 0.108 | −0.480 | −0.231 | −0.249 |
| HSbrain_94_l | 0.103 | −0.414 | −0.165 | −0.249 |
| HS_from_MQbrain_56_r | 0.140 | 0.263 | 0.512 | −0.249 |
| HS_264_r | 0.170 | −0.277 | −0.033 | −0.244 |
| HS_from_MQbrain_1222_r | 0.147 | −0.456 | −0.212 | −0.244 |
| hsa-mir-192_r | 0.170 | −0.033 | 0.211 | −0.243 |
| HSbrain_708_l | 0.095 | −0.485 | −0.243 | −0.242 |
| HS_from_MM_103_l | 0.099 | −0.154 | 0.089 | −0.242 |
| HS_from_MM_251_r | 0.113 | −0.289 | −0.047 | −0.242 |
| HS_168_r | 0.140 | −0.608 | −0.367 | −0.240 |
| HS_from_MQbrain_1832_l | 0.030 | −0.372 | −0.132 | −0.240 |
| HSbrain_433_r | 0.196 | 0.076 | 0.312 | −0.236 |
| HSbrain_210_l | 0.189 | −0.350 | −0.114 | −0.235 |
| HSbrain_522_r | 0.140 | −0.057 | 0.178 | −0.235 |
| HS_from_MQbrain_1574_l | 0.129 | −0.195 | 0.040 | −0.235 |
| HSbrain_471_r | 0.189 | −0.059 | 0.175 | −0.234 |
| HS_128_r | 0.170 | −0.408 | −0.174 | −0.234 |
| HS_57_l | 0.084 | −0.132 | 0.101 | −0.233 |
| Hsd_from_Mmd_261_l | 0.043 | −0.223 | 0.009 | −0.232 |
| HS_from_PT_308_l | 0.065 | −0.164 | 0.067 | −0.232 |
| Hsd_from_Mmd_235_r | 0.170 | 0.389 | 0.621 | −0.231 |
| HS_from_MM_339_r | 0.005 | −0.211 | 0.019 | −0.230 |
| Hsd_from_Mmd_436_l | 0.068 | 0.065 | 0.294 | −0.230 |
| hsa-mir-765_r | 0.170 | −0.628 | −0.399 | −0.229 |
| HS_from_MQbrain_406_r | 0.196 | −0.588 | −0.362 | −0.226 |
| HS_from_MQbrain_1204_r | 0.159 | −0.475 | −0.249 | −0.226 |
| hsa-mir-224_r | 0.113 | −0.139 | 0.087 | −0.226 |
| HS_from_MQbrain_1883_r | 0.196 | −0.343 | −0.118 | −0.225 |
| HS_from_MQbrain_1630_l | 0.009 | −0.228 | −0.005 | −0.223 |
| HS_25_r | 0.124 | −0.152 | 0.071 | −0.223 |
| HSbrain_152_l | 0.129 | −0.232 | −0.009 | −0.223 |
| HS_from_PT_238_l | 0.153 | −0.588 | −0.370 | −0.218 |
| HS_from_MQbrain_715_l | 0.084 | −0.218 | −0.001 | −0.217 |
| HS_from_MQbrain_1834_r | 0.119 | −0.402 | −0.186 | −0.216 |
| HSbrain_266_r | 0.038 | −0.094 | 0.121 | −0.215 |
| Hsd_from_Mmd_237_l | 0.124 | −0.154 | 0.061 | −0.215 |
| hsa-mir-211_r | 0.108 | −0.272 | −0.058 | −0.214 |
| Hsd_from_Mmd_371_r | 0.092 | −0.247 | −0.033 | −0.214 |
| HS_from_MQbrain_850_l | 0.183 | −0.340 | −0.127 | −0.214 |
| Hsd_from_Mmd_274_r | 0.189 | −0.159 | 0.054 | −0.213 |
| HS_from_MQbrain_597_l | 0.129 | −0.577 | −0.364 | −0.213 |
| HS_from_MQbrain_1750_r | 0.119 | −0.620 | −0.408 | −0.212 |
| HS_83_r | 0.170 | −0.107 | 0.103 | −0.210 |
| Hsd_63_r | 0.046 | −0.051 | 0.158 | −0.209 |
| HS_from_MQbrain_1349_l | 0.183 | −0.464 | −0.255 | −0.209 |
| HSbrain_402_l | 0.164 | −0.178 | 0.030 | −0.209 |
| Hsd_from_Mmd_78_r | 0.103 | −0.437 | −0.228 | −0.208 |
| HSbrain_452_l | 0.119 | −0.192 | 0.016 | −0.208 |
| HS_from_MQbrain_70_l | 0.061 | −0.336 | −0.131 | −0.206 |
| HS_from_MQbrain_1219_l | 0.072 | −0.421 | −0.216 | −0.205 |
| HS_from_MM_115_l | 0.056 | −0.242 | −0.037 | −0.204 |
| HSbrain_626_l | 0.176 | −0.007 | 0.197 | −0.204 |
| HS_from_MQbrain_281_l | 0.170 | −0.322 | −0.118 | −0.204 |
| HS_from_MQbrain_628_l | 0.176 | −0.131 | 0.071 | −0.202 |
| HS_from_PT_247_l | 0.189 | −0.250 | −0.049 | −0.201 |
| HS_119_l | 0.080 | −0.077 | 0.124 | −0.200 |
| HSbrain_783_l | 0.159 | −0.235 | −0.035 | −0.200 |
| HS_from_MQbrain_1355_r | 0.103 | −0.064 | 0.136 | −0.200 |
| HSbrain_485_r | 0.021 | −0.292 | −0.093 | −0.199 |
| HS_129_l | 0.095 | −0.341 | −0.143 | −0.198 |
| HSbrain_80_l | 0.170 | −0.128 | 0.069 | −0.198 |
| HS_from_MQbrain_1110_l | 0.140 | −0.055 | 0.142 | −0.197 |
| HS_130_l | 0.176 | −0.004 | 0.190 | −0.195 |
| HS_from_MQbrain_1846_r | 0.170 | −0.164 | 0.030 | −0.193 |
| HSbrain_347_l | 0.159 | −0.392 | −0.199 | −0.193 |
| HSbrain_548_l | 0.084 | −0.271 | −0.079 | −0.192 |
| HS_from_MQbrain_338_l | 0.153 | 0.011 | 0.202 | −0.191 |
| HSd_from_Mmd_52_r | 0.164 | −0.717 | −0.527 | −0.191 |
| HSbrain_328_l | 0.113 | −0.209 | −0.019 | −0.190 |
| HS_from_MQbrain_1228_r | 0.095 | −0.395 | −0.205 | −0.190 |
| HS_from_MM_124_l | 0.108 | −0.173 | 0.017 | −0.190 |
| HSbrain_216_r | 0.170 | −0.228 | −0.039 | −0.189 |
| HS_from_MQbrain_999_l | 0.119 | −0.229 | −0.040 | −0.189 |
| HSbrain_56_l | 0.084 | −0.150 | 0.039 | −0.189 |
| HS_from_MQbrain_996_l | 0.164 | −0.341 | −0.153 | −0.188 |
| HS_from_MQbrain_1509_r | 0.183 | −0.579 | −0.391 | −0.188 |
| HS_from_MQbrain_550_l | 0.092 | −0.294 | −0.106 | −0.188 |
| HSbrain_8_r | 0.024 | −0.390 | −0.202 | −0.188 |
| HS_from_MQbrain_1036_r | 0.147 | −0.463 | −0.276 | −0.187 |
| Hsd_from_Mmd_388_l | 0.135 | −0.135 | 0.051 | −0.186 |
| HS_from_MQbrain_566_r | 0.164 | −0.444 | −0.258 | −0.186 |
| HS_from_MQbrain_941_r | 0.164 | −0.208 | −0.023 | −0.186 |
| HS_from_MQbrain_1889_l | 0.119 | −0.155 | 0.030 | −0.185 |
| HS_from_MM_340_r | 0.164 | −0.442 | −0.257 | −0.185 |
| HS_from_PT_158_l | 0.159 | −0.475 | −0.291 | −0.185 |
| HS_from_MQbrain_1241_r | 0.095 | −0.169 | 0.014 | −0.183 |
| HSbrain_305_r | 0.099 | −0.303 | −0.121 | −0.182 |
| HS_from_MQbrain_732_r | 0.183 | −0.164 | 0.018 | −0.182 |
| HS_197_r | 0.170 | −0.206 | −0.025 | −0.181 |
| HS_from_MQbrain_1675_r | 0.147 | −0.400 | −0.218 | −0.181 |
| HS_from_MQbrain_707_l | 0.153 | −0.068 | 0.111 | −0.179 |
| HSbrain_222_r | 0.153 | 0.025 | 0.204 | −0.179 |
| HS_from_MQbrain_1380_r | 0.183 | −0.298 | −0.120 | −0.178 |
| HSbrain_275_r | 0.164 | 0.068 | 0.243 | −0.176 |
| HS_from_MQbrain_689_l | 0.153 | −0.177 | −0.001 | −0.175 |
| HS_from_MQbrain_449_l | 0.164 | −0.336 | −0.161 | −0.175 |
| HSbrain_306_r | 0.153 | −0.241 | −0.069 | −0.172 |
| HSbrain_810_l | 0.196 | −0.211 | −0.040 | −0.172 |
| HSbrain_240_l | 0.099 | −0.366 | −0.195 | −0.171 |
| HSbrain_769_r | 0.099 | −0.129 | 0.041 | −0.170 |
| HS_from_MQbrain_38_r | 0.056 | 0.045 | 0.213 | −0.168 |
| HS_from_MQbrain_546_l | 0.108 | −0.166 | 0.000 | −0.165 |
| HS_18_l | 0.159 | −0.250 | −0.087 | −0.164 |
| HSbrain_53_l | 0.170 | −0.221 | −0.058 | −0.163 |
| HS_from_PT_194_r | 0.135 | −0.001 | 0.161 | −0.162 |
| hsa-mir-647_r | 0.027 | −0.154 | 0.006 | −0.161 |
| HSbrain_599_r | 0.147 | −0.011 | 0.146 | −0.157 |
| HSbrain_4_l | 0.113 | −0.060 | 0.095 | −0.155 |
| HS_from_MQbrain_1523_r | 0.135 | −0.166 | −0.012 | −0.154 |
| HSbrain_70_r | 0.196 | −0.402 | −0.250 | −0.153 |
| Hsd_from_Mmd_197_l | 0.196 | −0.075 | 0.077 | −0.152 |
| HS_from_MQbrain_22_r | 0.140 | −0.252 | −0.104 | −0.148 |
| HS_from_MQbrain_487_r | 0.189 | −0.100 | 0.046 | −0.146 |
| HS_22_r | 0.129 | 0.023 | 0.168 | −0.144 |
| HSbrain_376_l | 0.164 | −0.016 | 0.126 | −0.142 |
| HSbrain_701_l | 0.170 | 0.014 | 0.155 | −0.141 |
| HS_from_MQbrain_43_l | 0.061 | 0.036 | 0.165 | −0.130 |
| Hsd_from_Mmd_128_l | 0.129 | 0.121 | 0.249 | −0.127 |
| hsa-mir-570_l | 0.140 | −0.103 | 0.020 | −0.123 |
| HS_from_MQbrain_810_l | 0.058 | −0.182 | −0.062 | −0.120 |
| HS_from_MQbrain_1863_l | 0.032 | 0.106 | 0.224 | −0.118 |
| HS_from_MQbrain_1223_l | 0.164 | −0.156 | −0.043 | −0.113 |
| Hsd_from_Mmd_333_l | 0.183 | −0.077 | 0.033 | −0.110 |
| HS_from_MQbrain_669_r | 0.084 | −0.015 | 0.089 | −0.103 |
| HS_from_MQbrain_1576_r | 0.183 | −0.006 | 0.095 | −0.100 |
| HS_from_MQbrain_97_l | 0.170 | −0.024 | 0.074 | −0.098 |
| HSbrain_541_r | 0.140 | −0.014 | 0.084 | −0.098 |
| HSbrain_545_r | 0.029 | −0.056 | 0.033 | −0.089 |
| HS_from_MQbrain_1018_l | 0.159 | −0.008 | 0.072 | −0.081 |
| HSbrain_469_l | 0.068 | 0.016 | 0.093 | −0.077 |
| HSbrain_727_l | 0.024 | 0.218 | 0.269 | −0.051 |
| Hsd_from_Mmd_259_r | 0.196 | 0.066 | 0.098 | −0.032 |
| HS_from_MQbrain_1674_l | 0.056 | −0.451 | −0.426 | −0.025 |
| HSbrain_723_l | 0.024 | 0.071 | 0.049 | 0.022 |
| Hsd_25_l | 0.196 | 0.017 | −0.038 | 0.054 |
| HS_from_MQbrain_1637_l | 0.108 | 0.088 | 0.027 | 0.061 |
| HSbrain_244_l | 0.129 | 0.095 | 0.032 | 0.062 |
| hsa-mir-507_l | 0.099 | 0.036 | −0.035 | 0.071 |
| HS_from_MQbrain_2087_r | 0.176 | 0.070 | −0.006 | 0.075 |
| Hsd_from_Mmd_325_r | 0.189 | 0.061 | −0.016 | 0.077 |
| hsa-mir-454_l | 0.196 | 0.034 | −0.044 | 0.078 |
| HS_286_r | 0.164 | 0.019 | −0.061 | 0.081 |
| Hsd_from_Mmd_391_r | 0.176 | 0.056 | −0.025 | 0.081 |
| HS_from_PT_196_l | 0.196 | 0.046 | −0.035 | 0.081 |
| hsa-mir-568_r | 0.017 | −0.020 | −0.105 | 0.085 |
| hsa-mir-518b_r | 0.099 | 0.030 | −0.056 | 0.086 |
| HS_from_MQbrain_1379_r | 0.040 | 0.049 | −0.038 | 0.086 |
| hsa-mir-139_l | 0.196 | −0.062 | −0.150 | 0.088 |
| Hsd_from_Mmd_500_r | 0.056 | 0.034 | −0.055 | 0.089 |
| HS_from_MQbrain_897_l | 0.119 | 0.080 | −0.008 | 0.089 |
| HS_from_MQbrain_2699_l | 0.159 | −0.016 | −0.106 | 0.089 |
| hsa-mir-33b_l | 0.164 | 0.024 | −0.067 | 0.091 |
| HSbrain_1076_l | 0.124 | 0.034 | −0.059 | 0.093 |
| hsa-mir-124a-1_l | 0.164 | 0.129 | 0.036 | 0.094 |
| HS_from_MQbrain_392_r | 0.170 | −0.007 | −0.104 | 0.097 |
| Hsd_from_Mmd_499_r | 0.119 | −0.033 | −0.131 | 0.098 |
| HS_from_MM_86_l | 0.189 | −0.144 | −0.243 | 0.099 |
| HS_from_MQbrain_779_l | 0.092 | 0.055 | −0.045 | 0.100 |
| Hsd_from_Mmd_316_r | 0.147 | 0.057 | −0.043 | 0.100 |
| HSbrain_478_r | 0.124 | 0.052 | −0.049 | 0.101 |
| HS_from_MM_109_l | 0.140 | 0.048 | −0.057 | 0.104 |
| hsa-mir-646_r | 0.058 | 0.078 | −0.027 | 0.105 |
| HS_from_MQbrain_1224_r | 0.189 | −0.013 | −0.118 | 0.105 |
| HS_from_MQbrain_1804_r | 0.135 | 0.061 | −0.045 | 0.106 |
| HS_262_r | 0.029 | 0.010 | −0.100 | 0.110 |
| HSbrain_780_l | 0.170 | 0.051 | −0.064 | 0.115 |
| HS_from_MQbrain_1687_l | 0.010 | −0.002 | −0.120 | 0.118 |
| HS_from_MQbrain_1721_l | 0.164 | 0.118 | −0.002 | 0.119 |
| HSbrain_1418_r | 0.176 | −0.023 | −0.143 | 0.121 |
| HS_from_MQbrain_221_r | 0.140 | 0.075 | −0.051 | 0.125 |
| Hsd_from_Mmd_190_r | 0.183 | 0.120 | −0.007 | 0.126 |
| HS_from_MM_57_r | 0.113 | 0.069 | −0.061 | 0.130 |
| HS_from_MM_150_r | 0.095 | 0.038 | −0.094 | 0.131 |
| HSbrain_590_l | 0.176 | 0.023 | −0.109 | 0.132 |
| HS_from_PT_99_r | 0.189 | 0.057 | −0.076 | 0.133 |
| HS_from_MQbrain_1164_l | 0.068 | 0.146 | 0.012 | 0.134 |
| Hsd_from_Mmd_431_l | 0.129 | 0.039 | −0.096 | 0.135 |
| HS_223_r | 0.170 | 0.137 | −0.001 | 0.138 |
| HS_from_PT_27_r | 0.176 | 0.155 | 0.014 | 0.140 |
| Hsd_from_Mmd_253_l | 0.189 | 0.013 | −0.128 | 0.141 |
| HS_from_PT_255_l | 0.159 | 0.038 | −0.106 | 0.144 |
| HS_from_MQbrain_1283_l | 0.050 | 0.125 | −0.021 | 0.146 |
| HSbrain_771_l | 0.176 | 0.400 | 0.252 | 0.147 |
| Hsd_from_Mmd_180_r | 0.189 | 0.232 | 0.084 | 0.148 |
| hsa-mir-432_r | 0.183 | 0.031 | −0.118 | 0.150 |
| hsa-mir-423_r | 0.036 | 0.179 | 0.022 | 0.156 |
| hsa-mir-1-2_l | 0.183 | −0.180 | −0.337 | 0.157 |
| HS_from_MQbrain_648_l | 0.196 | 0.152 | −0.005 | 0.157 |
| HSbrain_204_r | 0.129 | 0.080 | −0.082 | 0.163 |
| HSbrain_615_l | 0.196 | 0.011 | −0.152 | 0.163 |
| HSbrain_596_r | 0.103 | −0.097 | −0.261 | 0.164 |
| Hsd_from_Mmd_344_l | 0.099 | 0.068 | −0.098 | 0.166 |
| HS_from_MQbrain_1722_l | 0.129 | 0.368 | 0.201 | 0.167 |
| HS_from_MQbrain_675_r | 0.084 | −0.002 | −0.170 | 0.168 |
| HS_from_MQbrain_67_r | 0.043 | 0.127 | −0.043 | 0.170 |
| HSbrain_715_l | 0.135 | −0.021 | −0.191 | 0.170 |
| Hsd_38_r | 0.153 | 0.356 | 0.185 | 0.171 |
| HS_190_r | 0.189 | 0.206 | 0.034 | 0.171 |
| HS_219_l | 0.140 | 0.293 | 0.121 | 0.173 |
| hsa-mir-331_r | 0.030 | 0.070 | −0.103 | 0.173 |
| hsa-mir-99a_r | 0.103 | 0.047 | −0.127 | 0.173 |
| HS_from_MQbrain_1184_r | 0.124 | 0.643 | 0.464 | 0.179 |
| HSbrain_403_l | 0.189 | −0.069 | −0.251 | 0.181 |
| HS_77_l | 0.189 | 0.160 | −0.022 | 0.182 |
| HSbrain_714_l | 0.023 | 0.104 | −0.078 | 0.182 |
| HSbrain_792_r | 0.034 | 0.095 | −0.088 | 0.182 |
| HS_from_MQbrain_1382_l | 0.099 | 0.505 | 0.320 | 0.185 |
| HS_109_l | 0.129 | 0.348 | 0.162 | 0.186 |
| HS_from_MQbrain_1561_l | 0.072 | 0.162 | −0.026 | 0.187 |
| HS_292_l | 0.038 | 0.093 | −0.095 | 0.188 |
| HS_124_l | 0.196 | −0.062 | −0.251 | 0.189 |
| HS_from_PT_21_r | 0.046 | 0.060 | −0.130 | 0.191 |
| HS_from_MQbrain_824_r | 0.164 | 0.234 | 0.043 | 0.191 |
| HS_from_MQbrain_188_l | 0.196 | −0.006 | −0.200 | 0.194 |
| HSbrain_509_l | 0.113 | 0.014 | −0.180 | 0.194 |
| HSbrain_643_l | 0.108 | 0.178 | −0.017 | 0.195 |
| HSbrain_156_l | 0.189 | 0.172 | −0.024 | 0.196 |
| HS_from_MM_49_l | 0.056 | 0.366 | 0.168 | 0.198 |
| HS_from_MQbrain_1862_l | 0.061 | 0.022 | −0.177 | 0.199 |
| HS_from_MM_237_l | 0.099 | −0.083 | −0.283 | 0.200 |
| HSbrain_540_l | 0.099 | 0.105 | −0.097 | 0.202 |
| HS_from_MM_48_l | 0.189 | 0.191 | −0.013 | 0.204 |
| HS_from_MM_89_l | 0.153 | 0.102 | −0.105 | 0.207 |
| HS_77_r | 0.196 | 0.231 | 0.024 | 0.208 |
| HS_from_MQbrain_1794_r | 0.129 | 0.140 | −0.069 | 0.209 |
| Hsd_from_Mmd_204_l | 0.080 | 0.324 | 0.115 | 0.209 |
| HS_from_MQbrain_921_l | 0.113 | 0.359 | 0.150 | 0.209 |
| HSbrain_721_r | 0.070 | 0.103 | −0.106 | 0.209 |
| HS_from_MQbrain_468_l | 0.170 | 0.749 | 0.539 | 0.210 |
| HSbrain_220_r | 0.119 | 0.167 | −0.044 | 0.211 |
| HS_from_MM_79_l | 0.124 | 0.689 | 0.478 | 0.211 |
| HS_from_MQbrain_490_r | 0.124 | 0.500 | 0.288 | 0.212 |
| HS_from_MQbrain_1215_l | 0.070 | 0.094 | −0.118 | 0.212 |
| HS_from_MQbrain_901_l | 0.050 | 0.289 | 0.077 | 0.212 |
| Hsd_111_r | 0.103 | −0.224 | −0.436 | 0.212 |
| HSbrain_717_r | 0.153 | −0.106 | −0.318 | 0.213 |
| HS_from_PT_204_r | 0.196 | 0.033 | −0.180 | 0.213 |
| HSbrain_50_l | 0.124 | 0.286 | 0.072 | 0.214 |
| HS_191_l | 0.040 | 0.118 | −0.096 | 0.214 |
| hsa-mir-196b_r | 0.170 | −0.001 | −0.216 | 0.215 |
| HS_from_PT_63_r | 0.043 | 0.166 | −0.050 | 0.216 |
| HS_from_MQbrain_609_l | 0.103 | 0.669 | 0.452 | 0.217 |
| HS_from_MQbrain_461_l | 0.135 | 0.120 | −0.099 | 0.219 |
| HS_from_MQbrain_1366_l | 0.108 | 0.461 | 0.241 | 0.220 |
| HSbrain_171_l | 0.017 | 0.169 | −0.050 | 0.220 |
| hsa-mir-224_l | 0.072 | −0.139 | −0.359 | 0.221 |
| hsa-mir-181a-1_r | 0.135 | −0.157 | −0.378 | 0.221 |
| HSbrain_804_l | 0.164 | 0.142 | −0.079 | 0.221 |
| HS_from_MQbrain_1896_r | 0.153 | −0.102 | −0.324 | 0.222 |
| HS_from_MQbrain_383_l | 0.196 | 0.060 | −0.163 | 0.223 |
| Hsd_from_Mmd_215_r | 0.080 | 0.014 | −0.208 | 0.223 |
| HSbrain_266_l | 0.153 | 0.428 | 0.205 | 0.223 |
| hsa-mir-31_r | 0.135 | −0.060 | −0.283 | 0.224 |
| HS_from_MQbrain_266_r | 0.140 | 0.074 | −0.150 | 0.224 |
| HS_from_MM_92_r | 0.099 | 0.030 | −0.195 | 0.225 |
| HS_from_MQbrain_1590_l | 0.119 | 0.806 | 0.578 | 0.228 |
| HS_from_MQbrain_1724_l | 0.164 | 0.749 | 0.521 | 0.228 |
| HSbrain_641_r | 0.153 | −0.024 | −0.254 | 0.230 |
| HSbrain_553_l | 0.189 | 0.097 | −0.133 | 0.230 |
| HSbrain_248_l | 0.159 | 0.632 | 0.402 | 0.230 |
| HS_from_MQbrain_930_r | 0.077 | 0.318 | 0.087 | 0.230 |
| HSbrain_419_l | 0.056 | 0.178 | −0.053 | 0.230 |
| HS_from_MQbrain_414_l | 0.159 | 0.803 | 0.570 | 0.233 |
| hsa-mir-453_l | 0.030 | 0.185 | −0.049 | 0.233 |
| HSbrain_371_r | 0.070 | −0.018 | −0.252 | 0.234 |
| hsa-mir-604_l | 0.189 | 0.046 | −0.188 | 0.235 |
| HSbrain_231_l | 0.019 | 0.327 | 0.092 | 0.235 |
| HS_from_MQbrain_1318_l | 0.135 | 0.681 | 0.445 | 0.236 |
| HS_from_PT_148_l | 0.080 | −0.106 | −0.343 | 0.237 |
| Hsd_from_Mmd_232_r | 0.183 | −0.015 | −0.253 | 0.237 |
| HS_from_PT_38_l | 0.129 | −0.280 | −0.518 | 0.238 |
| HS_from_MQbrain_1030_l | 0.147 | 0.778 | 0.539 | 0.239 |
| Hsd_62_l | 0.068 | 0.623 | 0.383 | 0.240 |
| HS_from_PT_313_r | 0.084 | 0.047 | −0.193 | 0.240 |
| HS_from_MQbrain_1592_l | 0.092 | 0.731 | 0.491 | 0.240 |
| HSbrain_123_l | 0.058 | 0.178 | −0.062 | 0.241 |
| Hsd_from_Mmd_19_l | 0.164 | 0.089 | −0.152 | 0.241 |
| HSbrain_409_r | 0.061 | 0.059 | −0.182 | 0.241 |
| Hsd_19_l | 0.196 | 0.891 | 0.649 | 0.242 |
| HS_76_r | 0.147 | 0.057 | −0.185 | 0.242 |
| HS_from_MQbrain_1280_l | 0.072 | 0.244 | 0.002 | 0.242 |
| HSbrain_364_r | 0.170 | −0.099 | −0.341 | 0.242 |
| HS_from_MQbrain_1712_r | 0.129 | 0.783 | 0.540 | 0.243 |
| HS_from_MQbrain_1381_r | 0.147 | 0.154 | −0.090 | 0.243 |
| HSbrain_731_r | 0.024 | 0.232 | −0.011 | 0.243 |
| HSbrain_130_r | 0.153 | 0.137 | −0.106 | 0.244 |
| HSbrain_543_l | 0.124 | 0.114 | −0.129 | 0.244 |
| HS_from_MQbrain_1340_l | 0.036 | 0.326 | 0.082 | 0.244 |
| HS_from_PT_48_l | 0.196 | −0.395 | −0.640 | 0.245 |
| hsa-mir-617_l | 0.088 | −0.106 | −0.351 | 0.245 |
| HSbrain_506_r | 0.108 | −0.013 | −0.258 | 0.245 |
| HS_from_MQbrain_1487_r | 0.119 | 0.314 | 0.069 | 0.245 |
| HSbrain_374_r | 0.053 | 0.005 | −0.242 | 0.247 |
| HSbrain_467_l | 0.170 | 0.226 | −0.022 | 0.248 |
| HS_from_MQbrain_135_l | 0.176 | 0.234 | −0.016 | 0.250 |
| HS_from_MQbrain_936_r | 0.164 | 0.478 | 0.227 | 0.252 |
| HSbrain_580_r | 0.038 | 0.029 | −0.222 | 0.252 |
| HS_from_MQbrain_881_l | 0.183 | 0.282 | 0.030 | 0.252 |
| HS_from_MQbrain_283_r | 0.147 | −0.052 | −0.303 | 0.252 |
| HS_from_MQbrain_120_l | 0.189 | 0.210 | −0.042 | 0.252 |
| HS_from_PT_22_l | 0.103 | 0.076 | −0.176 | 0.253 |
| HSbrain_745_l | 0.170 | 0.591 | 0.337 | 0.254 |
| hsa-mir-324_l | 0.196 | 0.036 | −0.219 | 0.255 |
| Hsd_from_Mmd_519_r | 0.095 | −0.172 | −0.428 | 0.256 |
| HS_304_r | 0.061 | 0.774 | 0.518 | 0.256 |
| HS_from_MQbrain_1700_l | 0.080 | 0.739 | 0.482 | 0.257 |
| HS_from_MQbrain_1212_l | 0.164 | 0.012 | −0.245 | 0.257 |
| HS_from_MQbrain_604_l | 0.113 | 0.592 | 0.334 | 0.257 |
| HS_from_MM_194_l | 0.196 | −0.232 | −0.489 | 0.258 |
| HS_from_MQbrain_789_l | 0.124 | 0.489 | 0.231 | 0.258 |
| HSbrain_227_r | 0.113 | 0.233 | −0.025 | 0.258 |
| hsa-mir-518c_l | 0.159 | −0.072 | −0.331 | 0.259 |
| HS_189_r | 0.135 | 0.018 | −0.241 | 0.260 |
| HS_from_MQbrain_1443_r | 0.032 | 0.499 | 0.239 | 0.260 |
| HSbrain_144_r | 0.140 | 0.402 | 0.142 | 0.260 |
| HSbrain_212_l | 0.068 | 0.365 | 0.105 | 0.260 |
| HS_from_MM_145_r | 0.129 | 0.539 | 0.278 | 0.261 |
| HS_from_MQbrain_1412_l | 0.061 | 0.235 | −0.027 | 0.261 |
| HSbrain_480_r | 0.072 | 0.220 | −0.041 | 0.262 |
| HS_from_MM_182_r | 0.189 | 0.012 | −0.250 | 0.262 |
| HS_from_MQbrain_29_r | 0.070 | 0.169 | −0.093 | 0.262 |
| Hsd_from_Mmd_330_l | 0.070 | 0.724 | 0.461 | 0.263 |
| HS_from_MM_149_r | 0.183 | 0.014 | −0.249 | 0.263 |
| HS_from_MQbrain_1841_r | 0.070 | 0.453 | 0.190 | 0.263 |
| Hsd_from_Mmd_378_l | 0.092 | 0.016 | −0.248 | 0.264 |
| HSbrain_496_r | 0.135 | 0.107 | −0.158 | 0.265 |
| HS_from_MQbrain_1637_r | 0.080 | 0.669 | 0.404 | 0.265 |
| HS_from_PT_299_l | 0.030 | 0.560 | 0.295 | 0.266 |
| HSbrain_431_r | 0.068 | −0.043 | −0.310 | 0.266 |
| HS_from_MQbrain_36_r | 0.129 | 0.707 | 0.440 | 0.266 |
| HSbrain_627_r | 0.189 | −0.199 | −0.466 | 0.267 |
| HSbrain_752_l | 0.153 | 0.618 | 0.351 | 0.267 |
| HS_from_MQbrain_1761_r | 0.010 | 0.063 | −0.204 | 0.267 |
| HS_from_MQbrain_1686_l | 0.050 | 0.556 | 0.287 | 0.268 |
| HS_from_MQbrain_1593_r | 0.084 | 0.010 | −0.259 | 0.269 |
| HSbrain_527_r | 0.021 | 0.188 | −0.082 | 0.270 |
| HSbrain_680_l | 0.164 | 0.643 | 0.371 | 0.271 |
| HS_from_MQbrain_207_l | 0.011 | 0.419 | 0.146 | 0.273 |
| HS_from_MM_34_l | 0.099 | 0.070 | −0.204 | 0.274 |
| HS_from_MQbrain_1658_l | 0.092 | −0.003 | −0.277 | 0.274 |
| hsa-mir-588_r | 0.135 | −0.038 | −0.313 | 0.274 |
| hsa-mir-599_r | 0.140 | −0.219 | −0.494 | 0.275 |
| HS_from_MQbrain_239_r | 0.068 | 0.200 | −0.077 | 0.277 |
| HS_from_MQbrain_631_l | 0.159 | 0.217 | −0.060 | 0.277 |
| HS_41_r | 0.140 | 0.115 | −0.163 | 0.277 |
| hsa-mir-770_r | 0.113 | 0.414 | 0.136 | 0.278 |
| HS_from_PT_43_l | 0.040 | 0.556 | 0.277 | 0.279 |
| HS_from_MM_289_r | 0.072 | −0.112 | −0.391 | 0.279 |
| HS_from_MM_184_r | 0.092 | 0.056 | −0.224 | 0.280 |
| Hsd_from_Mmd_344_r | 0.176 | 0.401 | 0.121 | 0.280 |
| HSbrain_130_l | 0.147 | −0.089 | −0.370 | 0.281 |
| HSbrain_361_l | 0.140 | 0.500 | 0.216 | 0.283 |
| HS_from_MQbrain_770_r | 0.092 | 0.133 | −0.150 | 0.283 |
| HS_from_PT_328_l | 0.189 | 0.916 | 0.633 | 0.283 |
| HS_from_MQbrain_1090_r | 0.164 | 0.785 | 0.502 | 0.284 |
| HS_from_MQbrain_1640_l | 0.135 | −0.162 | −0.446 | 0.284 |
| HSbrain_113_r | 0.095 | 0.246 | −0.038 | 0.284 |
| HSbrain_706_l | 0.183 | 0.144 | −0.140 | 0.284 |
| HS_74_r | 0.164 | 0.022 | −0.262 | 0.284 |
| HS_from_MM_229_r | 0.088 | 0.239 | −0.047 | 0.286 |
| HSbrain_661_l | 0.080 | 0.295 | 0.009 | 0.286 |
| HSbrain_299_r | 0.108 | 0.337 | 0.051 | 0.286 |
| HS_from_MQbrain_234_r | 0.135 | 0.095 | −0.192 | 0.287 |
| HSbrain_677_l | 0.103 | 0.580 | 0.292 | 0.287 |
| HS_306_r | 0.099 | 0.086 | −0.201 | 0.288 |
| HS_from_MQbrain_1642_l | 0.050 | 0.194 | −0.094 | 0.289 |
| HS_from_MQbrain_84_r | 0.196 | −0.016 | −0.306 | 0.290 |
| HSbrain_139_r | 0.147 | 0.028 | −0.263 | 0.290 |
| HS_from_MM_331_r | 0.189 | 0.087 | −0.204 | 0.291 |
| HS_from_MQbrain_540_l | 0.135 | 0.527 | 0.235 | 0.292 |
| HSbrain_271_l | 0.077 | 0.277 | −0.016 | 0.292 |
| HSbrain_446_r | 0.164 | 0.229 | −0.064 | 0.293 |
| hsa-mir-133a-2_r | 0.015 | 0.045 | −0.248 | 0.293 |
| HS_from_MQbrain_828_r | 0.113 | −0.077 | −0.370 | 0.293 |
| HS_from_MM_165_l | 0.088 | 0.274 | −0.019 | 0.293 |
| HS_115_r | 0.153 | 0.076 | −0.217 | 0.293 |
| Hsd_from_Mmd_280_l | 0.153 | 0.068 | −0.227 | 0.294 |
| HS_from_PT_178_l | 0.140 | 0.365 | 0.070 | 0.295 |
| HS_from_MQbrain_1706_l | 0.080 | 0.011 | −0.284 | 0.295 |
| HSbrain_145_l | 0.119 | 0.537 | 0.241 | 0.296 |
| HS_73_r | 0.113 | 0.093 | −0.203 | 0.296 |
| HS_from_MQbrain_769_r | 0.084 | −0.227 | −0.524 | 0.297 |
| HS_from_MQbrain_1759_l | 0.147 | −0.484 | −0.781 | 0.297 |
| Hsd_18_l | 0.024 | 0.264 | −0.034 | 0.298 |
| HS_from_MQbrain_951_l | 0.040 | −0.184 | −0.483 | 0.298 |
| HS_from_PT_237_l | 0.124 | 0.091 | −0.207 | 0.299 |
| HS_from_MM_1_l | 0.070 | 0.044 | −0.255 | 0.299 |
| HS_109_r | 0.058 | 0.140 | −0.159 | 0.299 |
| HS_95_r | 0.159 | 0.115 | −0.185 | 0.300 |
| hsa-mir-622_r | 0.129 | 0.125 | −0.175 | 0.300 |
| hsa-mir-550-2_r | 0.038 | 0.054 | −0.246 | 0.301 |
| HS_from_MQbrain_883_r | 0.058 | 0.108 | −0.193 | 0.301 |
| HSbrain_624_l | 0.147 | 0.406 | 0.105 | 0.301 |
| hsa-mir-550-1_r | 0.135 | 0.070 | −0.231 | 0.301 |
| HSbrain_811_l | 0.153 | 0.249 | −0.053 | 0.301 |
| HS_from_MM_124_r | 0.183 | 0.245 | −0.057 | 0.302 |
| Hsd_from_Mmd_97_l | 0.036 | 0.019 | −0.283 | 0.302 |
| Hsd_70_l | 0.025 | 0.301 | −0.001 | 0.302 |
| HS_from_MM_84_r | 0.140 | 0.278 | −0.024 | 0.303 |
| HS_from_MM_119_r | 0.135 | 0.196 | −0.107 | 0.303 |
| hsa-mir-409_r | 0.119 | −0.062 | −0.366 | 0.304 |
| Hsd_77_r | 0.147 | 0.582 | 0.277 | 0.304 |
| HSbrain_84_l | 0.050 | 0.033 | −0.271 | 0.304 |
| HS_from_MM_232_r | 0.170 | −0.130 | −0.435 | 0.305 |
| HS_66_r | 0.077 | 0.016 | −0.289 | 0.305 |
| HS_from_MQbrain_260_r | 0.147 | 0.919 | 0.614 | 0.305 |
| HS_from_MQbrain_592_l | 0.119 | 0.560 | 0.255 | 0.305 |
| HSbrain_237_l | 0.196 | 0.083 | −0.222 | 0.305 |
| HSbrain_301_l | 0.072 | −0.048 | −0.354 | 0.306 |
| HS_from_MQbrain_155_l | 0.159 | 0.415 | 0.109 | 0.306 |
| hsa-mir-190_r | 0.183 | −0.207 | −0.513 | 0.306 |
| HS_from_MQbrain_1260_r | 0.005 | 0.078 | −0.228 | 0.306 |
| HS_111_l | 0.140 | 0.182 | −0.125 | 0.307 |
| HSbrain_788_l | 0.077 | −0.018 | −0.326 | 0.308 |
| HSbrain_250_r | 0.072 | 0.183 | −0.125 | 0.308 |
| HSbrain_415_l | 0.099 | 0.852 | 0.544 | 0.308 |
| HSbrain_673_l | 0.159 | 0.614 | 0.306 | 0.309 |
| Hsd_82_r | 0.119 | 0.092 | −0.217 | 0.309 |
| HS_from_MQbrain_1602_l | 0.092 | 0.607 | 0.298 | 0.309 |
| HS_from_MQbrain_1459_l | 0.103 | 0.242 | −0.068 | 0.311 |
| HS_from_MQbrain_1771_r | 0.070 | 0.467 | 0.156 | 0.311 |
| HSbrain_300_l | 0.058 | −0.034 | −0.345 | 0.312 |
| HSbrain_728_l | 0.070 | 0.048 | −0.265 | 0.313 |
| HSbrain_495_r | 0.113 | 0.278 | −0.036 | 0.314 |
| HS_from_MQbrain_67_l | 0.147 | 0.278 | −0.037 | 0.314 |
| HSbrain_473_l | 0.095 | 0.066 | −0.249 | 0.314 |
| HS_121_r | 0.140 | −0.247 | −0.562 | 0.315 |
| Hsd_from_Mmd_480_r | 0.099 | 0.393 | 0.078 | 0.315 |
| HS_from_MQbrain_768_l | 0.189 | −0.116 | −0.431 | 0.315 |
| HS_from_PT_237_r | 0.013 | 0.375 | 0.060 | 0.315 |
| Hsd_from_Mmd_524_r | 0.068 | −0.074 | −0.391 | 0.317 |
| HSbrain_1425_r | 0.080 | 0.033 | −0.284 | 0.317 |
| HS_281_r | 0.001 | 0.024 | −0.293 | 0.317 |
| HS_from_MQbrain_650_r | 0.164 | 0.647 | 0.329 | 0.318 |
| hsa-mir-30d_l | 0.099 | −0.193 | −0.511 | 0.318 |
| HSbrain_671_r | 0.056 | 0.006 | −0.314 | 0.319 |
| HS_from_MQbrain_1684_r | 0.092 | 0.219 | −0.100 | 0.319 |
| HSbrain_55_l | 0.159 | 0.203 | −0.117 | 0.319 |
| HS_from_MQbrain_739_l | 0.070 | 0.043 | −0.276 | 0.319 |
| Hsd_from_Mmd_49_l | 0.048 | 0.374 | 0.054 | 0.320 |
| HS_from_MQbrain_291_l | 0.088 | 0.671 | 0.350 | 0.320 |
| HSbrain_598_l | 0.065 | 0.319 | −0.002 | 0.320 |
| hsa-mir-133a-1_r | 0.034 | 0.140 | −0.180 | 0.321 |
| HSbrain_408_r | 0.061 | 0.099 | −0.223 | 0.322 |
| HS_from_PT_220_l | 0.108 | 0.774 | 0.452 | 0.322 |
| HS_from_MQbrain_997_l | 0.070 | 0.705 | 0.383 | 0.322 |
| HS_from_MQbrain_502_r | 0.119 | −0.061 | −0.384 | 0.322 |
| HS_from_MQbrain_882_l | 0.103 | 0.123 | −0.200 | 0.323 |
| hsa-mir-296_l | 0.016 | 0.082 | −0.243 | 0.325 |
| HS_from_MQbrain_1446_l | 0.065 | 0.056 | −0.269 | 0.325 |
| Hsd_20_r | 0.099 | 0.121 | −0.204 | 0.325 |
| HSbrain_597_r | 0.023 | 0.707 | 0.382 | 0.325 |
| HS_from_MQbrain_1513_r | 0.036 | −0.284 | −0.609 | 0.325 |
| HSbrain_214_l | 0.061 | 0.195 | −0.131 | 0.326 |
| HS_from_MQbrain_444_r | 0.027 | 0.273 | −0.053 | 0.326 |
| HS_from_MQbrain_1744_r | 0.103 | 0.357 | 0.031 | 0.326 |
| HSbrain_722_l | 0.024 | 0.457 | 0.130 | 0.327 |
| HS_from_MQbrain_1884_r | 0.010 | 0.365 | 0.038 | 0.327 |
| HS_289_l | 0.119 | 0.887 | 0.559 | 0.328 |
| HS_from_MQbrain_1885_r | 0.092 | 0.295 | −0.033 | 0.329 |
| Hsd_from_Mmd_93_r | 0.092 | 0.393 | 0.064 | 0.329 |
| HSbrain_716_l | 0.140 | 0.504 | 0.175 | 0.329 |
| HS_from_PT_161_l | 0.183 | −0.016 | −0.345 | 0.329 |
| Hsd_from_Mmd_298_l | 0.095 | 0.067 | −0.263 | 0.330 |
| HS_214_r | 0.135 | −0.146 | −0.475 | 0.330 |
| HS_from_PT_27_l | 0.103 | 0.062 | −0.268 | 0.330 |
| HS_from_MM_121_l | 0.027 | 0.010 | −0.320 | 0.330 |
| HS_11_l | 0.088 | 0.176 | −0.154 | 0.330 |
| hsa-mir-609_r | 0.135 | 0.112 | −0.219 | 0.331 |
| HS_from_MQbrain_684_r | 0.135 | 0.173 | −0.159 | 0.332 |
| HS_from_MQbrain_1427_l | 0.068 | 0.511 | 0.179 | 0.332 |
| Hsd_from_Mmd_44_r | 0.159 | −0.038 | −0.370 | 0.333 |
| hsa-mir-296_r | 0.103 | 0.509 | 0.177 | 0.333 |
| HSbrain_482_l | 0.103 | 0.381 | 0.048 | 0.333 |
| HS_from_MQbrain_1545_r | 0.095 | −0.248 | −0.581 | 0.333 |
| Hsd_from_Mmd_410_r | 0.036 | 0.062 | −0.272 | 0.335 |
| HSbrain_1338_r | 0.065 | 0.062 | −0.272 | 0.335 |
| HS_from_PT_299_r | 0.036 | 0.123 | −0.212 | 0.335 |
| HS_from_MQbrain_467_l | 0.140 | 0.164 | −0.171 | 0.335 |
| Hsd_from_Mmd_527_l | 0.040 | 0.075 | −0.261 | 0.335 |
| hsa-mir-625_r | 0.095 | 0.258 | −0.078 | 0.336 |
| HS_from_MQbrain_1250_r | 0.147 | 0.014 | −0.322 | 0.336 |
| HS_from_MQbrain_723_l | 0.053 | 0.126 | −0.211 | 0.336 |
| HSbrain_95_l | 0.164 | 0.001 | −0.336 | 0.337 |
| hsa-mir-526b_r | 0.108 | −0.393 | −0.731 | 0.338 |
| HS_from_PT_70_r | 0.046 | 0.477 | 0.139 | 0.338 |
| HSbrain_1410_l | 0.043 | 0.613 | 0.275 | 0.338 |
| HS_from_MQbrain_698_r | 0.077 | 0.215 | −0.124 | 0.338 |
| HSbrain_784_r | 0.164 | 0.646 | 0.308 | 0.338 |
| hsa-mir-767_r | 0.050 | 0.043 | −0.296 | 0.339 |
| HSbrain_696_r | 0.030 | 0.116 | −0.223 | 0.340 |
| HS_from_MQbrain_1445_l | 0.032 | 0.638 | 0.298 | 0.340 |
| HS_from_MQbrain_1684_l | 0.027 | 0.214 | −0.126 | 0.340 |
| hsa-mir-323_l | 0.016 | 0.418 | 0.077 | 0.341 |
| HS_from_MM_295_r | 0.072 | 0.439 | 0.098 | 0.341 |
| HS_from_MQbrain_482_l | 0.034 | 0.019 | −0.322 | 0.341 |
| Hsd_79_l | 0.095 | 1.169 | 0.828 | 0.341 |
| HSbrain_143_l | 0.170 | 0.885 | 0.543 | 0.342 |
| hsa-mir-93_l | 0.183 | −0.474 | −0.816 | 0.342 |
| HSbrain_75_l | 0.024 | 0.433 | 0.089 | 0.344 |
| HSbrain_124_l | 0.088 | 0.821 | 0.477 | 0.345 |
| Hsd_from_Mmd_489_r | 0.124 | 0.044 | −0.302 | 0.345 |
| hsa-mir-554_l | 0.017 | 0.159 | −0.187 | 0.346 |
| HS_from_MQbrain_1776_l | 0.129 | 0.783 | 0.437 | 0.346 |
| HSbrain_180_l | 0.196 | 0.131 | −0.215 | 0.346 |
| HS_from_MQbrain_1267_r | 0.088 | 0.024 | −0.323 | 0.346 |
| HSbrain_272_l | 0.036 | 0.028 | −0.319 | 0.347 |
| HS_from_MQbrain_154_r | 0.153 | 0.268 | −0.079 | 0.347 |
| HS_from_MQbrain_1855_l | 0.140 | 0.079 | −0.269 | 0.348 |
| hsa-mir-412_r | 0.061 | 0.236 | −0.114 | 0.350 |
| HS_from_MQbrain_848_l | 0.088 | 0.124 | −0.227 | 0.350 |
| HSbrain_652_l | 0.050 | 0.616 | 0.265 | 0.350 |
| HS_287_l | 0.124 | 0.889 | 0.538 | 0.351 |
| HS_from_PT_102_l | 0.080 | 0.854 | 0.502 | 0.352 |
| Hsd_from_Mmd_101_r | 0.135 | 0.182 | −0.170 | 0.352 |
| HSbrain_637_l | 0.092 | 0.704 | 0.351 | 0.353 |
| HS_from_PT_196_r | 0.164 | 0.197 | −0.157 | 0.353 |
| HSbrain_92_l | 0.040 | 0.572 | 0.219 | 0.353 |
| HS_from_MQbrain_253_r | 0.072 | 0.028 | −0.326 | 0.354 |
| HS_from_MQbrain_664_l | 0.056 | 0.763 | 0.409 | 0.354 |
| hsa-let-7f-1_r | 0.092 | −0.042 | −0.397 | 0.355 |
| HS_from_MQbrain_900_r | 0.159 | 0.051 | −0.304 | 0.355 |
| HS_from_MQbrain_664_r | 0.140 | 0.304 | −0.051 | 0.355 |
| HS_4_r | 0.092 | 0.812 | 0.456 | 0.356 |
| hsa-mir-1-1_l | 0.147 | −0.033 | −0.389 | 0.356 |
| HS_from_MQbrain_1632_r | 0.010 | 0.174 | −0.182 | 0.356 |
| HS_from_MQbrain_282_l | 0.038 | 0.489 | 0.132 | 0.357 |
| hsa-mir-506_r | 0.092 | −0.034 | −0.392 | 0.358 |
| HS_from_MQbrain_1760_r | 0.189 | 0.864 | 0.504 | 0.359 |
| HS_from_MQbrain_251_r | 0.070 | 0.860 | 0.500 | 0.360 |
| hsa-mir-500_l | 0.029 | −0.234 | −0.594 | 0.360 |
| HS_from_MQbrain_1745_r | 0.080 | 0.373 | 0.013 | 0.360 |
| HS_from_MQbrain_343_l | 0.050 | 0.119 | −0.241 | 0.360 |
| Hsd_from_Mmd_161_l | 0.092 | 0.865 | 0.505 | 0.360 |
| HSbrain_1337_l | 0.080 | 0.129 | −0.232 | 0.361 |
| HSbrain_242_l | 0.189 | −0.254 | −0.616 | 0.362 |
| Hsd_from_Mmd_217_r | 0.119 | −0.028 | −0.390 | 0.362 |
| HS_59_l | 0.056 | 0.579 | 0.216 | 0.363 |
| hsa-mir-98_r | 0.159 | −0.065 | −0.429 | 0.364 |
| HS_from_MQbrain_131_r | 0.007 | 0.219 | −0.146 | 0.365 |
| hsa-mir-628_r | 0.061 | 0.184 | −0.181 | 0.365 |
| HS_from_MM_304_r | 0.129 | 0.878 | 0.512 | 0.366 |
| HS_from_MQbrain_1253_r | 0.084 | −0.014 | −0.380 | 0.366 |
| hsa-mir-134_r | 0.030 | 0.237 | −0.130 | 0.366 |
| hsa-mir-148b_r | 0.058 | −0.018 | −0.384 | 0.367 |
| Hsd_18_r | 0.053 | 0.102 | −0.264 | 0.367 |
| HSbrain_472_l | 0.020 | 0.126 | −0.242 | 0.367 |
| HS_from_MQbrain_1126_l | 0.176 | −0.380 | −0.748 | 0.368 |
| HS_from_PT_280_l | 0.164 | 0.002 | −0.367 | 0.369 |
| hsa-mir-532_l | 0.099 | 0.005 | −0.365 | 0.370 |
| hsa-mir-521-2_r | 0.088 | 0.301 | −0.069 | 0.370 |
| hsa-mir-106b_r | 0.032 | −0.338 | −0.708 | 0.370 |
| HS_from_MQbrain_1227_l | 0.043 | 0.034 | −0.337 | 0.371 |
| hsa-mir-379_l | 0.077 | 0.882 | 0.511 | 0.371 |
| Hsd_from_Mmd_291_r | 0.108 | 0.939 | 0.568 | 0.371 |
| HS_from_MM_32_r | 0.048 | 0.081 | −0.291 | 0.371 |
| HSbrain_722_r | 0.099 | 0.202 | −0.170 | 0.372 |
| HSbrain_388_r | 0.183 | 0.180 | −0.192 | 0.372 |
| hsa-mir-616_l | 0.070 | 0.022 | −0.350 | 0.373 |
| HS_from_PT_146_r | 0.013 | 0.243 | −0.130 | 0.373 |
| Hsd_102_l | 0.036 | 0.430 | 0.056 | 0.374 |
| hsa-mir-641_r | 0.153 | −0.082 | −0.456 | 0.374 |
| HS_from_MQbrain_74_r | 0.029 | 0.718 | 0.344 | 0.374 |
| HSbrain_121_l | 0.159 | 0.626 | 0.252 | 0.374 |
| HS_from_PT_95_r | 0.092 | 0.960 | 0.585 | 0.375 |
| HSbrain_1360_l | 0.092 | 0.947 | 0.572 | 0.375 |
| HS_from_MQbrain_1450_r | 0.065 | 0.418 | 0.042 | 0.376 |
| HS_268_l | 0.147 | 0.175 | −0.201 | 0.376 |
| Hsd_17_l | 0.124 | 1.038 | 0.662 | 0.376 |
| HS_from_PT_50_r | 0.095 | 0.346 | −0.031 | 0.376 |
| Hsd_from_Mmd_333_r | 0.072 | 0.268 | −0.108 | 0.376 |
| HSbrain_1363_l | 0.072 | 0.910 | 0.534 | 0.376 |
| HS_from_MQbrain_841_l | 0.170 | 0.990 | 0.613 | 0.377 |
| HSbrain_720_l | 0.099 | 0.115 | −0.262 | 0.377 |
| Hsd_74_r | 0.135 | 0.166 | −0.212 | 0.377 |
| HSbrain_570_l | 0.124 | 0.507 | 0.129 | 0.378 |
| HSbrain_396_r | 0.124 | 0.104 | −0.275 | 0.379 |
| HS_from_MQbrain_56_l | 0.056 | 0.687 | 0.307 | 0.379 |
| HS_from_MQbrain_549_r | 0.068 | 0.182 | −0.198 | 0.380 |
| hsa-mir-659_l | 0.017 | 0.060 | −0.320 | 0.380 |
| HS_from_MQbrain_128_r | 0.061 | 0.606 | 0.226 | 0.381 |
| HS_213_l | 0.080 | 0.138 | −0.243 | 0.381 |
| HSbrain_225_r | 0.019 | 0.308 | −0.073 | 0.381 |
| HS_from_MM_202_l | 0.084 | −0.296 | −0.677 | 0.381 |
| HSbrain_1413_l | 0.017 | 0.287 | −0.094 | 0.381 |
| HS_from_PT_235_l | 0.070 | −0.047 | −0.429 | 0.382 |
| HS_from_MQbrain_1213_l | 0.099 | 0.632 | 0.250 | 0.382 |
| HS_from_MQbrain_542_r | 0.050 | 0.330 | −0.054 | 0.384 |
| HS_from_MQbrain_1703_r | 0.040 | 0.010 | −0.374 | 0.384 |
| HS_from_MQbrain_806_l | 0.088 | −0.347 | −0.731 | 0.384 |
| hsa-mir-151_l | 0.159 | 0.139 | −0.245 | 0.384 |
| HSbrain_114_r | 0.030 | 0.080 | −0.305 | 0.385 |
| HS_from_MQbrain_800_r | 0.034 | 0.393 | 0.007 | 0.386 |
| HS_from_MQbrain_88_l | 0.108 | −0.341 | −0.727 | 0.386 |
| HS_288_l | 0.095 | 0.923 | 0.537 | 0.386 |
| HS_from_MQbrain_1462_r | 0.147 | −0.133 | −0.519 | 0.386 |
| HS_from_MQbrain_1781_l | 0.092 | 0.038 | −0.348 | 0.386 |
| HS_from_MQbrain_1234_l | 0.189 | 0.323 | −0.064 | 0.387 |
| Hsd_from_Mmd_334_r | 0.159 | 0.078 | −0.309 | 0.387 |
| HS_from_MM_354_r | 0.038 | 0.226 | −0.162 | 0.388 |
| HS_212_l | 0.048 | 0.075 | −0.313 | 0.388 |
| Hsd_from_Mmd_166_r | 0.095 | −0.192 | −0.581 | 0.389 |
| HS_from_PT_219_l | 0.147 | 0.844 | 0.455 | 0.389 |
| HSbrain_359_l | 0.027 | 0.281 | −0.108 | 0.389 |
| hsa-mir-92-1_l | 0.176 | 0.482 | 0.092 | 0.389 |
| HSbrain_1364_l | 0.072 | 0.934 | 0.545 | 0.390 |
| HSbrain_74_r | 0.024 | 0.209 | −0.181 | 0.390 |
| HSbrain_749_r | 0.072 | 0.610 | 0.218 | 0.392 |
| HS_from_MQbrain_796_l | 0.058 | 0.183 | −0.209 | 0.393 |
| HSbrain_504_r | 0.103 | 0.288 | −0.105 | 0.393 |
| HS_from_MQbrain_1831_r | 0.159 | 0.310 | −0.083 | 0.393 |
| HS_from_MQbrain_693_r | 0.153 | 1.457 | 1.064 | 0.393 |
| HS_from_MM_6_l | 0.183 | 0.197 | −0.197 | 0.393 |
| HS_281_r | 0.001 | 0.083 | −0.312 | 0.395 |
| HS_from_MQbrain_1808_r | 0.010 | 0.575 | 0.180 | 0.395 |
| Hsd_6_l | 0.053 | 0.033 | −0.362 | 0.395 |
| HS_from_MQbrain_55_l | 0.011 | 0.103 | −0.292 | 0.395 |
| HS_from_MM_342_r | 0.068 | 0.301 | −0.094 | 0.395 |
| hsa-mir-521-1_l | 0.147 | 0.094 | −0.302 | 0.396 |
| HS_from_MQbrain_509_r | 0.027 | −0.223 | −0.619 | 0.396 |
| HS_from_MQbrain_641_r | 0.070 | 0.072 | −0.325 | 0.397 |
| HSbrain_711_l | 0.053 | 0.239 | −0.158 | 0.397 |
| HS_from_MQbrain_209_r | 0.050 | 0.217 | −0.180 | 0.398 |
| HS_from_MQbrain_1097_r | 0.135 | 0.614 | 0.217 | 0.398 |
| hsa-mir-641_l | 0.080 | −0.273 | −0.674 | 0.400 |
| hsa-mir-624_l | 0.183 | −0.154 | −0.555 | 0.401 |
| HS_164_l | 0.108 | 0.684 | 0.284 | 0.401 |
| hsa-mir-23a_l | 0.140 | 0.530 | 0.129 | 0.401 |
| HS_from_MQbrain_1747_r | 0.040 | 0.184 | −0.217 | 0.401 |
| HS_56_r | 0.040 | 0.327 | −0.074 | 0.401 |
| Hsd_from_Mmd_328_r | 0.124 | 0.199 | −0.202 | 0.401 |
| HS_204_l | 0.113 | −0.449 | −0.850 | 0.401 |
| HS_from_PT_271_l | 0.147 | 0.142 | −0.260 | 0.402 |
| HS_114_l | 0.048 | 0.800 | 0.399 | 0.402 |
| HS_from_MQbrain_154_l | 0.084 | 0.912 | 0.509 | 0.403 |
| HS_from_MQbrain_1376_l | 0.070 | 0.643 | 0.239 | 0.404 |
| HS_from_MQbrain_555_r | 0.129 | 0.281 | −0.123 | 0.404 |
| Hsd_from_Mmd_478_l | 0.108 | 0.326 | −0.078 | 0.404 |
| hsa-mir-513-2_l | 0.124 | 0.153 | −0.251 | 0.404 |
| HS_from_PT_20_r | 0.084 | 0.660 | 0.256 | 0.404 |
| HS_from_PT_187_r | 0.058 | 0.122 | −0.283 | 0.405 |
| HSbrain_483_l | 0.021 | 0.160 | −0.245 | 0.405 |
| Hsd_from_Mmd_422_l | 0.046 | 0.067 | −0.340 | 0.407 |
| HS_from_MQbrain_1354_l | 0.061 | 0.424 | 0.016 | 0.408 |
| HSbrain_330_l | 0.068 | 0.856 | 0.448 | 0.408 |
| HS_150_l | 0.061 | 0.702 | 0.294 | 0.409 |
| HS_from_MQbrain_1493_r | 0.147 | 1.414 | 1.005 | 0.409 |
| hsa-mir-634_l | 0.119 | 0.595 | 0.184 | 0.410 |
| Hsd_from_Mmd_23_l | 0.036 | −0.045 | −0.455 | 0.410 |
| HSbrain_158_l | 0.017 | 0.272 | −0.139 | 0.411 |
| HS_from_MQbrain_97_r | 0.147 | 0.512 | 0.101 | 0.411 |
| HSbrain_425_l | 0.140 | −0.124 | −0.537 | 0.412 |
| hsa-mir-219-1_r | 0.072 | −0.224 | −0.636 | 0.412 |
| HSbrain_448_l | 0.129 | 0.690 | 0.277 | 0.413 |
| HSbrain_171_l | 0.017 | 0.374 | −0.039 | 0.413 |
| HS_from_PT_32_r | 0.013 | 0.064 | −0.349 | 0.413 |
| HSbrain_572_r | 0.068 | −0.109 | −0.523 | 0.414 |
| Hsd_from_Mmd_208_r | 0.183 | −0.459 | −0.874 | 0.415 |
| hsa-let-7a-2_r | 0.040 | 0.127 | −0.290 | 0.416 |
| HS_from_MQbrain_1377_l | 0.170 | 0.499 | 0.083 | 0.416 |
| HS_from_MM_195_r | 0.084 | 0.990 | 0.574 | 0.417 |
| hsa-mir-652_r | 0.164 | −0.221 | −0.638 | 0.418 |
| HSbrain_50_r | 0.029 | 0.027 | −0.391 | 0.418 |
| hsa-mir-662_l | 0.196 | 1.115 | 0.697 | 0.419 |
| HSbrain_619_r | 0.092 | −0.156 | −0.576 | 0.419 |
| HS_from_MQbrain_1199_l | 0.170 | 1.278 | 0.858 | 0.420 |
| HS_from_MQbrain_723_r | 0.070 | 0.837 | 0.417 | 0.421 |
| Hsd_from_Mmd_325_l | 0.099 | −0.149 | −0.570 | 0.421 |
| Hsd_42_r | 0.043 | 0.390 | −0.031 | 0.421 |
| HSbrain_604_r | 0.153 | 0.110 | −0.313 | 0.422 |
| HS_from_PT_101_r | 0.013 | 0.168 | −0.255 | 0.423 |
| HS_131_r | 0.053 | 0.774 | 0.349 | 0.424 |
| hsa-mir-30c-2_l | 0.164 | −0.140 | −0.564 | 0.424 |
| HS_from_MQbrain_948_r | 0.088 | 0.369 | −0.055 | 0.424 |
| HS_from_MQbrain_1695_r | 0.147 | 0.274 | −0.150 | 0.424 |
| HSbrain_1362_l | 0.048 | 0.903 | 0.478 | 0.425 |
| HS_from_PT_121_r | 0.058 | 0.745 | 0.320 | 0.425 |
| HSbrain_108_l | 0.140 | 0.073 | −0.354 | 0.427 |
| HS_from_MQbrain_1717_r | 0.046 | 0.133 | −0.294 | 0.427 |
| HSbrain_768_l | 0.159 | 0.729 | 0.302 | 0.427 |
| hsa-mir-653_l | 0.046 | 0.091 | −0.336 | 0.427 |
| HS_166_r | 0.077 | 0.573 | 0.145 | 0.428 |
| HS_from_MM_289_l | 0.043 | 0.004 | −0.424 | 0.428 |
| HS_from_MQbrain_782_r | 0.043 | 1.069 | 0.639 | 0.430 |
| HS_from_MQbrain_1658_r | 0.072 | 0.047 | −0.383 | 0.430 |
| HSbrain_35_r | 0.058 | 0.689 | 0.259 | 0.431 |
| HS_186_r | 0.176 | 0.490 | 0.058 | 0.432 |
| HS_from_MQbrain_1201_r | 0.095 | 0.803 | 0.371 | 0.432 |
| hsa-mir-518f_r | 0.068 | −0.162 | −0.595 | 0.433 |
| HS_53_r | 0.140 | 1.218 | 0.785 | 0.434 |
| HS_118_l | 0.032 | 0.766 | 0.332 | 0.435 |
| HS_from_MQbrain_412_l | 0.103 | −0.533 | −0.968 | 0.435 |
| Hsd_62_l | 0.068 | 0.256 | −0.179 | 0.435 |
| HSbrain_1377_l | 0.017 | 0.175 | −0.261 | 0.436 |
| HSbrain_420_r | 0.020 | 0.485 | 0.049 | 0.436 |
| HS_from_MQbrain_566_l | 0.011 | 0.175 | −0.260 | 0.436 |
| HSbrain_6_r | 0.023 | 0.587 | 0.151 | 0.437 |
| HS_from_MQbrain_1392_r | 0.032 | 0.978 | 0.541 | 0.437 |
| HS_from_MQbrain_105_l | 0.113 | −0.007 | −0.443 | 0.437 |
| Hsd_from_Mmd_123_l | 0.140 | 0.183 | −0.253 | 0.437 |
| HS_from_MQbrain_105_l | 0.113 | 0.102 | −0.335 | 0.437 |
| HSbrain_797_r | 0.080 | 0.079 | −0.358 | 0.437 |
| HS_from_MQbrain_940_r | 0.013 | 0.435 | −0.002 | 0.437 |
| hsa-mir-633_r | 0.119 | −0.150 | −0.589 | 0.439 |
| HS_from_MQbrain_6_r | 0.153 | 0.882 | 0.443 | 0.439 |
| hsa-mir-648_l | 0.088 | 0.768 | 0.328 | 0.439 |
| HS_from_MQbrain_1700_r | 0.056 | 0.279 | −0.160 | 0.440 |
| HS_from_PT_340_l | 0.061 | 0.116 | −0.325 | 0.441 |
| HS_61_l | 0.095 | −0.372 | −0.814 | 0.442 |
| HSbrain_520_l | 0.113 | 1.209 | 0.767 | 0.442 |
| HSbrain_658_l | 0.196 | 0.001 | −0.442 | 0.443 |
| HS_from_MQbrain_969_l | 0.034 | 0.265 | −0.178 | 0.443 |
| HS_from_MM_157_l | 0.129 | −0.293 | −0.736 | 0.443 |
| HS_from_MQbrain_1794_l | 0.053 | 0.300 | −0.144 | 0.443 |
| HS_from_MQbrain_1404_r | 0.061 | 0.787 | 0.343 | 0.444 |
| hsa-mir-521-1_r | 0.056 | 0.323 | −0.121 | 0.444 |
| Hsd_from_Mmd_187_r | 0.183 | 0.428 | −0.016 | 0.445 |
| HS_from_MQbrain_1414_r | 0.077 | 0.603 | 0.158 | 0.445 |
| HSbrain_713_l | 0.030 | 0.842 | 0.397 | 0.445 |
| HS_from_MQbrain_873_r | 0.053 | 1.085 | 0.639 | 0.445 |
| Hsd_19_r | 0.058 | 0.191 | −0.254 | 0.446 |
| HS_from_MQbrain_1773_l | 0.048 | 0.663 | 0.217 | 0.446 |
| HS_from_MQbrain_1807_l | 0.147 | 0.503 | 0.056 | 0.447 |
| hsa-mir-30b_l | 0.070 | 0.049 | −0.398 | 0.447 |
| HS_from_MQbrain_315_l | 0.050 | 0.531 | 0.083 | 0.448 |
| HS_from_MQbrain_1395_r | 0.053 | 0.783 | 0.334 | 0.449 |
| HSbrain_507_l | 0.072 | 0.081 | −0.369 | 0.449 |
| HS_from_MQbrain_1696_l | 0.065 | −0.575 | −1.025 | 0.449 |
| HS_from_MQbrain_100_l | 0.005 | 0.316 | −0.134 | 0.450 |
| Hsd_from_Mmd_92_l | 0.176 | 1.146 | 0.696 | 0.450 |
| HS_from_MQbrain_1239_l | 0.053 | −0.109 | −0.560 | 0.451 |
| HS_from_MQbrain_1388_l | 0.043 | 1.152 | 0.701 | 0.451 |
| hsa-mir-630_r | 0.099 | −0.056 | −0.509 | 0.452 |
| HS_190_l | 0.058 | 0.834 | 0.381 | 0.453 |
| hsa-mir-375_l | 0.088 | 0.394 | −0.059 | 0.453 |
| HS_from_MQbrain_2380_r | 0.011 | 0.503 | 0.049 | 0.454 |
| HS_from_PT_339_r | 0.095 | −0.091 | −0.545 | 0.454 |
| Hsd_from_Mmd_70_l | 0.058 | 0.351 | −0.104 | 0.455 |
| Hsd_from_Mmd_519_l | 0.025 | 0.065 | −0.390 | 0.455 |
| HS_from_MQbrain_1520_r | 0.080 | 0.629 | 0.173 | 0.456 |
| HSbrain_413_l | 0.159 | −0.215 | −0.671 | 0.456 |
| HSbrain_610_r | 0.070 | 0.783 | 0.327 | 0.456 |
| HS_from_MQbrain_676_r | 0.183 | 1.535 | 1.079 | 0.456 |
| HS_from_MQbrain_1565_r | 0.072 | 0.564 | 0.106 | 0.458 |
| HS_from_MM_160_l | 0.183 | −0.049 | −0.507 | 0.458 |
| HS_from_MQbrain_349_l | 0.011 | 0.160 | −0.301 | 0.461 |
| HS_from_PT_250_r | 0.135 | 1.045 | 0.583 | 0.462 |
| HSbrain_102_r | 0.017 | 0.677 | 0.215 | 0.462 |
| hsa-mir-329-1_r | 0.068 | −0.013 | −0.476 | 0.463 |
| hsa-mir-135a-2_r | 0.015 | 0.264 | −0.199 | 0.463 |
| Hsd_from_Mmd_367_l | 0.170 | −0.305 | −0.769 | 0.464 |
| HS_from_MQbrain_576_l | 0.080 | 0.570 | 0.106 | 0.465 |
| HS_from_MQbrain_917_r | 0.013 | −0.163 | −0.628 | 0.465 |
| HSbrain_794_r | 0.024 | 0.758 | 0.292 | 0.466 |
| HSbrain_295_r | 0.196 | −0.277 | −0.743 | 0.466 |
| HSbrain_280_l | 0.108 | 1.581 | 1.114 | 0.467 |
| HS_33_l | 0.038 | 0.378 | −0.091 | 0.469 |
| hsa-mir-329-2_r | 0.040 | 0.039 | −0.430 | 0.469 |
| HSbrain_506_l | 0.065 | 0.253 | −0.218 | 0.471 |
| hsa-mir-193a_l | 0.070 | 1.119 | 0.648 | 0.471 |
| HS_from_MQbrain_498_r | 0.038 | 1.096 | 0.624 | 0.472 |
| HS_from_MM_319_l | 0.004 | 0.890 | 0.418 | 0.472 |
| HS_from_MQbrain_854_r | 0.012 | 0.170 | −0.303 | 0.473 |
| HSbrain_148_r | 0.017 | 0.430 | −0.043 | 0.473 |
| HSbrain_705_r | 0.147 | 1.198 | 0.724 | 0.474 |
| HS_from_MQbrain_191_l | 0.010 | 0.502 | 0.028 | 0.475 |
| hsa-mir-331_l | 0.070 | −0.128 | −0.603 | 0.475 |
| hsa-let-7c_r | 0.040 | 0.146 | −0.330 | 0.475 |
| HS_from_MQbrain_20_r | 0.011 | −0.133 | −0.608 | 0.475 |
| HSbrain_700_r | 0.038 | 0.218 | −0.259 | 0.477 |
| HS_from_MQbrain_350_l | 0.011 | 0.166 | −0.311 | 0.478 |
| hsa-mir-513-1_l | 0.099 | 0.213 | −0.265 | 0.478 |
| hsa-mir-299_l | 0.016 | 0.126 | −0.354 | 0.480 |
| HS_from_MQbrain_791_r | 0.189 | −0.470 | −0.951 | 0.481 |
| hsa-mir-144_r | 0.176 | −0.446 | −0.927 | 0.481 |
| HSbrain_754_r | 0.024 | 0.162 | −0.320 | 0.481 |
| HSbrain_165_l | 0.034 | 1.001 | 0.519 | 0.482 |
| hsa-mir-412_l | 0.129 | 0.195 | −0.288 | 0.483 |
| hsa-mir-422a_l | 0.159 | 1.153 | 0.670 | 0.483 |
| HSbrain_317_l | 0.080 | 0.542 | 0.059 | 0.483 |
| HS_from_MQbrain_529_r | 0.164 | 0.548 | 0.065 | 0.483 |
| HS_from_MQbrain_375_l | 0.011 | 0.196 | −0.288 | 0.484 |
| HS_from_MM_47_l | 0.068 | 0.020 | −0.464 | 0.484 |
| Hsd_from_Mmd_480_l | 0.025 | 0.193 | −0.291 | 0.484 |
| HS_189_l | 0.040 | 0.881 | 0.395 | 0.485 |
| HS_from_MM_72_r | 0.056 | −0.012 | −0.498 | 0.486 |
| Hsd_from_Mmd_527_r | 0.025 | −0.455 | −0.942 | 0.487 |
| HS_from_MQbrain_224_l | 0.077 | 0.677 | 0.190 | 0.487 |
| HS_from_MQbrain_1456_l | 0.009 | 0.744 | 0.256 | 0.488 |
| HS_from_MQbrain_655_l | 0.011 | 0.084 | −0.404 | 0.489 |
| HS_from_MQbrain_1363_l | 0.007 | 0.297 | −0.192 | 0.490 |
| HSbrain_1076_r | 0.017 | 0.188 | −0.303 | 0.491 |
| hsa-mir-155_l | 0.103 | −0.206 | −0.697 | 0.491 |
| HS_85_l | 0.129 | 1.342 | 0.850 | 0.492 |
| HS_from_MM_18_l | 0.004 | 0.221 | −0.273 | 0.494 |
| HS_from_MQbrain_1384_r | 0.124 | 0.458 | −0.038 | 0.496 |
| Hsd_46_l | 0.025 | 0.367 | −0.129 | 0.496 |
| hsa-let-7c_l | 0.099 | −0.464 | −0.964 | 0.500 |
| HS_from_MQbrain_288_r | 0.011 | 0.196 | −0.306 | 0.502 |
| HS_from_MQbrain_951_l | 0.040 | 0.919 | 0.416 | 0.503 |
| HS_307_r | 0.183 | −0.456 | −0.959 | 0.503 |
| HSbrain_557_r | 0.095 | 1.309 | 0.804 | 0.504 |
| hsa-mir-516-4_l | 0.016 | 0.429 | −0.076 | 0.505 |
| hsa-let-7d_l | 0.029 | −0.159 | −0.664 | 0.505 |
| HS_78_r | 0.170 | 0.725 | 0.220 | 0.505 |
| Hsd_26_l | 0.025 | 0.212 | −0.293 | 0.505 |
| HS_79_l | 0.004 | 0.213 | −0.293 | 0.505 |
| HS_from_MQbrain_1064_r | 0.088 | 0.645 | 0.139 | 0.506 |
| Hsd_from_Mmd_93_l | 0.119 | 1.178 | 0.672 | 0.507 |
| HS_from_MQbrain_1522_r | 0.009 | 0.032 | −0.476 | 0.508 |
| HS_from_MQbrain_468_r | 0.068 | 0.332 | −0.176 | 0.509 |
| HS_from_MQbrain_750_r | 0.065 | 0.894 | 0.385 | 0.509 |
| hsa-mir-516-3_l | 0.016 | 0.360 | −0.151 | 0.511 |
| Hsd_from_Mmd_410_l | 0.170 | −0.321 | −0.832 | 0.511 |
| Hsd_47_r | 0.170 | 1.086 | 0.574 | 0.512 |
| HS_from_MQbrain_969_r | 0.153 | 0.561 | 0.048 | 0.512 |
| HS_from_PT_136_l | 0.050 | 1.198 | 0.686 | 0.513 |
| HSbrain_709_l | 0.023 | 0.841 | 0.327 | 0.513 |
| HS_from_MQbrain_1028_l | 0.164 | 1.570 | 1.057 | 0.513 |
| HSbrain_585_l | 0.050 | 0.904 | 0.390 | 0.514 |
| hsa-mir-125a_r | 0.014 | 0.758 | 0.243 | 0.515 |
| HS_from_MM_178_r | 0.004 | 0.170 | −0.345 | 0.515 |
| hsa-mir-135a-1_r | 0.140 | 0.615 | 0.100 | 0.515 |
| Hsd_from_Mmd_176_l | 0.099 | 0.008 | −0.508 | 0.516 |
| HS_from_MQbrain_1844_r | 0.043 | 0.622 | 0.106 | 0.516 |
| HSbrain_259_r | 0.030 | 0.708 | 0.192 | 0.516 |
| HSbrain_261_l | 0.038 | 0.190 | −0.326 | 0.516 |
| hsa-mir-497_r | 0.016 | 0.312 | −0.205 | 0.517 |
| HS_from_MQbrain_1692_l | 0.135 | 1.678 | 1.160 | 0.518 |
| HSbrain_359_r | 0.020 | 0.957 | 0.437 | 0.520 |
| HS_from_MM_295_l | 0.004 | 0.192 | −0.328 | 0.520 |
| Hsd_from_Mmd_302_r | 0.029 | 0.158 | −0.363 | 0.521 |
| HS_from_PT_74_r | 0.095 | 1.193 | 0.672 | 0.521 |
| HS_from_MQbrain_774_r | 0.050 | 0.494 | −0.027 | 0.521 |
| hsa-mir-587_r | 0.189 | −0.141 | −0.663 | 0.522 |
| hsa-mir-383_l | 0.183 | 0.011 | −0.513 | 0.524 |
| HSbrain_458_l | 0.034 | −0.681 | −1.206 | 0.525 |
| HSbrain_321_r | 0.019 | 0.716 | 0.191 | 0.525 |
| hsa-mir-125b-1_r | 0.108 | 0.830 | 0.304 | 0.526 |
| HS_from_MQbrain_330_r | 0.011 | 0.687 | 0.161 | 0.526 |
| hsa-mir-183_r | 0.189 | 1.388 | 0.861 | 0.526 |
| HS_from_MM_347_l | 0.088 | 1.264 | 0.738 | 0.527 |
| Hsd_37_l | 0.053 | 1.037 | 0.510 | 0.528 |
| HS_from_MQbrain_1485_l | 0.009 | 0.128 | −0.400 | 0.528 |
| hsa-mir-502_l | 0.092 | −0.266 | −0.794 | 0.528 |
| HS_15_l | 0.043 | 1.549 | 1.020 | 0.529 |
| HSbrain_72_l | 0.129 | 1.342 | 0.812 | 0.529 |
| hsa-mir-10a_r | 0.103 | 0.156 | −0.375 | 0.531 |
| HS_106_l | 0.030 | 0.650 | 0.119 | 0.532 |
| hsa-mir-514-3_r | 0.183 | −0.317 | −0.849 | 0.532 |
| HSbrain_691_l | 0.095 | −0.348 | −0.881 | 0.533 |
| HS_from_MQbrain_1417_l | 0.092 | 0.735 | 0.199 | 0.536 |
| HSbrain_318_l | 0.029 | 0.876 | 0.338 | 0.537 |
| HS_from_MQbrain_472_r | 0.048 | 0.832 | 0.294 | 0.538 |
| HS_from_MM_87_l | 0.124 | −0.292 | −0.830 | 0.538 |
| HS_from_MQbrain_671_r | 0.011 | 1.040 | 0.502 | 0.538 |
| HS_from_MQbrain_1072_r | 0.153 | −0.043 | −0.582 | 0.539 |
| HS_217_l | 0.001 | 0.658 | 0.118 | 0.540 |
| HS_from_PT_234_l | 0.164 | 0.188 | −0.352 | 0.540 |
| HS_from_MQbrain_1304_r | 0.124 | 0.507 | −0.034 | 0.542 |
| HS_from_MQbrain_1393_r | 0.007 | 0.873 | 0.331 | 0.542 |
| HSbrain_617_l | 0.023 | 1.098 | 0.556 | 0.542 |
| HSbrain_276_l | 0.088 | 0.101 | −0.443 | 0.544 |
| HS_161_r | 0.140 | −0.342 | −0.886 | 0.544 |
| hsa-mir-137_l | 0.068 | 0.471 | −0.075 | 0.546 |
| Hsd_from_Mmd_223_r | 0.095 | −0.374 | −0.922 | 0.548 |
| HS_from_MM_298_l | 0.189 | −0.268 | −0.817 | 0.549 |
| HSbrain_811_r | 0.024 | −0.031 | −0.580 | 0.549 |
| hsa-mir-188_l | 0.153 | 0.494 | −0.056 | 0.549 |
| Hsd_from_Mmd_439_l | 0.061 | −0.262 | −0.811 | 0.549 |
| HS_from_MM_313_l | 0.099 | −0.694 | −1.245 | 0.551 |
| HS_from_MQbrain_1139_r | 0.099 | −0.337 | −0.890 | 0.552 |
| Hsd_from_Mmd_34_r | 0.046 | 0.650 | 0.095 | 0.555 |
| HS_from_MQbrain_629_l | 0.011 | 0.906 | 0.350 | 0.556 |
| HSbrain_503_r | 0.021 | 0.793 | 0.236 | 0.557 |
| Hsd_from_Mmd_501_r | 0.084 | 0.618 | 0.060 | 0.558 |
| HS_from_MQbrain_147_l | 0.183 | 0.715 | 0.156 | 0.559 |
| hsa-mir-598_l | 0.027 | 0.957 | 0.398 | 0.559 |
| HS_180_r | 0.065 | 0.240 | −0.319 | 0.559 |
| HSbrain_663_r | 0.147 | −0.320 | −0.880 | 0.560 |
| HS_215_l | 0.050 | 1.344 | 0.784 | 0.560 |
| Hsd_11_l | 0.080 | 0.507 | −0.054 | 0.561 |
| Hsd_96_r | 0.147 | −0.209 | −0.772 | 0.563 |
| HS_from_MQbrain_441_l | 0.011 | 0.246 | −0.320 | 0.565 |
| HS_from_MQbrain_1297_l | 0.007 | 0.454 | −0.113 | 0.566 |
| Hsd_from_Mmd_219_r | 0.025 | −0.010 | −0.577 | 0.567 |
| HSbrain_536_r | 0.108 | −0.351 | −0.919 | 0.569 |
| HSbrain_286_l | 0.084 | 1.278 | 0.709 | 0.569 |
| Hsd_from_Mmd_314_r | 0.189 | −0.315 | −0.885 | 0.569 |
| HS_from_MQbrain_212_r | 0.011 | 0.049 | −0.523 | 0.573 |
| HSbrain_455_r | 0.080 | 1.623 | 1.049 | 0.573 |
| hsa-mir-101-1_r | 0.176 | −0.476 | −1.050 | 0.574 |
| HS_from_MQbrain_384_r | 0.084 | −0.235 | −0.809 | 0.574 |
| Hsd_from_Mmd_249_r | 0.108 | 0.047 | −0.528 | 0.575 |
| HS_from_MQbrain_6_l | 0.159 | 1.944 | 1.368 | 0.576 |
| HS_from_MQbrain_625_l | 0.159 | 0.255 | −0.323 | 0.577 |
| HSbrain_546_r | 0.030 | 0.510 | −0.070 | 0.581 |
| hsa-mir-95_r | 0.030 | −0.158 | −0.739 | 0.581 |
| HSbrain_131_l | 0.124 | 1.302 | 0.720 | 0.582 |
| HS_from_MQbrain_1300_r | 0.007 | −0.097 | −0.680 | 0.583 |
| hsa-mir-506_l | 0.070 | −0.318 | −0.901 | 0.583 |
| hsa-mir-203_r | 0.189 | 0.895 | 0.310 | 0.584 |
| hsa-mir-668_l | 0.050 | 1.334 | 0.749 | 0.585 |
| Hsd_from_Mmd_68_l | 0.183 | −0.614 | −1.200 | 0.587 |
| hsa-mir-432_l | 0.176 | 0.356 | −0.232 | 0.588 |
| HS_from_MQbrain_435_l | 0.034 | 0.566 | −0.022 | 0.588 |
| HS_from_MQbrain_1080_l | 0.040 | −0.105 | −0.694 | 0.589 |
| HS_from_MQbrain_1501_r | 0.159 | 1.467 | 0.877 | 0.590 |
| Hsd_from_Mmd_518_r | 0.119 | 0.521 | −0.070 | 0.591 |
| hsa-mir-30c-1_l | 0.036 | −0.002 | −0.594 | 0.592 |
| HS_from_MQbrain_839_r | 0.012 | 0.055 | −0.537 | 0.592 |
| HS_from_MQbrain_1625_r | 0.009 | 0.910 | 0.318 | 0.592 |
| HSbrain_801_r | 0.024 | 0.462 | −0.130 | 0.592 |
| HSbrain_267_r | 0.129 | 0.470 | −0.123 | 0.593 |
| HS_from_MQbrain_2670_r | 0.099 | 0.673 | 0.079 | 0.594 |
| HS_from_MQbrain_68_r | 0.159 | −0.182 | −0.776 | 0.594 |
| hsa-mir-571_r | 0.113 | −0.086 | −0.680 | 0.594 |
| hsa-mir-616_r | 0.017 | −0.025 | −0.620 | 0.595 |
| HS_from_MQbrain_1610_r | 0.050 | 1.004 | 0.409 | 0.595 |
| HS_from_MQbrain_549_l | 0.011 | 0.234 | −0.361 | 0.595 |
| HS_156_l | 0.058 | −0.022 | −0.618 | 0.595 |
| HS_from_MQbrain_1037_r | 0.092 | 0.808 | 0.213 | 0.595 |
| HSbrain_543_r | 0.038 | 0.457 | −0.139 | 0.596 |
| hsa-mir-125a_l | 0.014 | 0.117 | −0.480 | 0.597 |
| HSbrain_268_r | 0.019 | 0.831 | 0.233 | 0.598 |
| HSbrain_399_l | 0.020 | 0.270 | −0.327 | 0.598 |
| HS_from_MQbrain_1292_r | 0.007 | 0.078 | −0.521 | 0.599 |
| Hsd_from_Mmd_92_r | 0.025 | 0.770 | 0.168 | 0.602 |
| HS_from_MQbrain_1300_l | 0.058 | 1.357 | 0.754 | 0.603 |
| HS_from_MQbrain_1056_l | 0.005 | −0.166 | −0.771 | 0.605 |
| HS_from_MQbrain_1695_l | 0.113 | −0.143 | −0.751 | 0.607 |
| HS_from_MQbrain_1315_l | 0.038 | 1.524 | 0.913 | 0.611 |
| HS_from_MM_309_r | 0.004 | 0.695 | 0.083 | 0.612 |
| HS_from_MQbrain_1469_r | 0.032 | −0.333 | −0.945 | 0.612 |
| HS_from_MQbrain_1665_r | 0.080 | 0.501 | −0.111 | 0.613 |
| Hsd_from_Mmd_123_r | 0.176 | 0.949 | 0.335 | 0.614 |
| hsa-mir-27b_r | 0.140 | −0.809 | −1.423 | 0.614 |
| HS_from_MQbrain_389_l | 0.196 | 0.842 | 0.226 | 0.616 |
| HS_from_MQbrain_1657_r | 0.010 | 0.032 | −0.586 | 0.618 |
| HSbrain_778_r | 0.024 | −0.435 | −1.053 | 0.618 |
| HS_151_r | 0.170 | −0.548 | −1.168 | 0.619 |
| Hsd_76_r | 0.053 | 0.546 | −0.074 | 0.620 |
| Hsd_7_r | 0.058 | 0.261 | −0.359 | 0.620 |
| HS_from_PT_22_l | 0.103 | 0.699 | 0.077 | 0.621 |
| hsa-mir-655_l | 0.017 | 0.099 | −0.523 | 0.622 |
| hsa-mir-302d_l | 0.016 | −0.382 | −1.009 | 0.626 |
| HSbrain_245_r | 0.070 | 1.023 | 0.396 | 0.627 |
| Hsd_from_Mmd_311_l | 0.068 | −0.222 | −0.850 | 0.628 |
| HSbrain_41_l | 0.034 | 0.714 | 0.086 | 0.628 |
| HSbrain_635_l | 0.072 | 1.042 | 0.413 | 0.628 |
| HS_218_r | 0.001 | 0.740 | 0.110 | 0.630 |
| HS_from_MQbrain_92_l | 0.048 | 1.368 | 0.731 | 0.637 |
| HS_from_MM_84_l | 0.176 | −0.246 | −0.884 | 0.639 |
| hsa-mir-514-1_r | 0.058 | −0.315 | −0.954 | 0.639 |
| hsa-mir-325_l | 0.016 | 0.410 | −0.231 | 0.641 |
| Hsd_65_r | 0.176 | 1.939 | 1.298 | 0.641 |
| HS_141_l | 0.001 | 1.087 | 0.444 | 0.643 |
| HSbrain_267_l | 0.113 | 0.441 | −0.203 | 0.644 |
| hsa-mir-652_l | 0.056 | 0.524 | −0.122 | 0.646 |
| HS_from_MQbrain_382_r | 0.159 | 1.932 | 1.286 | 0.646 |
| Hsd_from_Mmd_318_l | 0.099 | −0.552 | −1.199 | 0.647 |
| HS_from_MM_241_l | 0.004 | −0.739 | −1.386 | 0.648 |
| HSbrain_767_r | 0.077 | 0.727 | 0.079 | 0.648 |
| HS_from_MQbrain_470_l | 0.040 | 1.293 | 0.645 | 0.648 |
| Hsd_from_Mmd_455_l | 0.164 | −0.593 | −1.248 | 0.655 |
| HSbrain_776_l | 0.065 | 1.480 | 0.821 | 0.659 |
| hsa-mir-526a-1_r | 0.176 | −0.304 | −0.964 | 0.660 |
| hsa-mir-520b_r | 0.053 | −0.035 | −0.696 | 0.661 |
| HSbrain_243_l | 0.019 | 0.648 | −0.014 | 0.661 |
| HS_from_MQbrain_751_l | 0.103 | 0.570 | −0.094 | 0.663 |
| HS_from_MQbrain_1628_r | 0.009 | 0.216 | −0.451 | 0.667 |
| Hsd_from_Mmd_35_l | 0.170 | 1.367 | 0.700 | 0.667 |
| HS_from_MM_185_r | 0.135 | 0.727 | 0.059 | 0.668 |
| HS_from_PT_254_r | 0.088 | 1.031 | 0.363 | 0.668 |
| hsa-mir-140_l | 0.038 | −0.345 | −1.022 | 0.677 |
| HS_from_MQbrain_883_l | 0.038 | 0.781 | 0.099 | 0.683 |
| HSbrain_540_r | 0.021 | 0.504 | −0.178 | 0.683 |
| HS_from_MQbrain_1485_r | 0.009 | 0.440 | −0.243 | 0.683 |
| HS_from_MQbrain_619_r | 0.011 | 0.807 | 0.123 | 0.685 |
| HS_from_MQbrain_1311_r | 0.007 | 0.146 | −0.542 | 0.688 |
| Hsd_from_Mmd_183_r | 0.124 | 1.219 | 0.529 | 0.690 |
| HSbrain_106_r | 0.017 | 0.772 | 0.081 | 0.691 |
| Hsd_from_Mmd_166_l | 0.058 | −0.375 | −1.071 | 0.696 |
| HS_from_MQbrain_1031_l | 0.092 | 1.334 | 0.638 | 0.696 |
| hsa-mir-573_l | 0.017 | −0.081 | −0.781 | 0.700 |
| HS_from_MQbrain_539_r | 0.048 | 1.051 | 0.345 | 0.705 |
| HSbrain_970_r | 0.129 | −0.385 | −1.091 | 0.706 |
| HS_from_MQbrain_1143_r | 0.046 | 0.735 | 0.029 | 0.706 |
| HSbrain_239_l | 0.056 | 0.875 | 0.169 | 0.706 |
| HS_from_MQbrain_71_r | 0.011 | 1.050 | 0.341 | 0.708 |
| HS_from_MM_63_l | 0.005 | 0.331 | −0.382 | 0.714 |
| HS_232_l | 0.001 | 1.105 | 0.389 | 0.716 |
| HS_36_l | 0.061 | 0.394 | −0.323 | 0.716 |
| HSbrain_426_r | 0.020 | 0.553 | −0.164 | 0.717 |
| hsa-mir-520f_r | 0.183 | −0.259 | −0.981 | 0.721 |
| hsa-mir-141_r | 0.050 | 0.053 | −0.670 | 0.723 |
| HS_from_PT_257_r | 0.013 | 0.313 | −0.410 | 0.723 |
| HS_from_MQbrain_255_l | 0.095 | 0.976 | 0.251 | 0.725 |
| hsa-mir-199a-2_r | 0.072 | −0.373 | −1.102 | 0.729 |
| HS_from_PT_190_r | 0.013 | 1.545 | 0.815 | 0.730 |
| Hsd_17_r | 0.032 | 0.906 | 0.175 | 0.732 |
| HSbrain_737_r | 0.140 | 0.684 | −0.048 | 0.732 |
| HSbrain_383_l | 0.119 | −1.312 | −2.045 | 0.732 |
| HS_from_MM_35_l | 0.159 | 1.499 | 0.757 | 0.743 |
| hsa-mir-17_l | 0.153 | −0.669 | −1.413 | 0.744 |
| HS_from_MQbrain_1100_l | 0.005 | 0.617 | −0.128 | 0.745 |
| HSbrain_170_r | 0.088 | −0.538 | −1.284 | 0.746 |
| hsa-mir-373_r | 0.029 | 0.750 | 0.003 | 0.748 |
| HS_from_MQbrain_691_l | 0.050 | 1.657 | 0.909 | 0.748 |
| hsa-mir-199a-1_r | 0.080 | −0.277 | −1.025 | 0.748 |
| HS_177_l | 0.043 | 0.069 | −0.680 | 0.748 |
| HSbrain_590_r | 0.088 | 0.695 | −0.053 | 0.748 |
| hsa-mir-378_r | 0.029 | 1.004 | 0.252 | 0.752 |
| hsa-mir-299_r | 0.061 | 0.678 | −0.075 | 0.753 |
| HS_from_MQbrain_1410_l | 0.099 | 0.318 | −0.438 | 0.755 |
| HS_from_PT_40_r | 0.013 | 0.229 | −0.528 | 0.757 |
| Hsd_from_Mmd_436_r | 0.040 | 0.764 | 0.005 | 0.759 |
| hsa-mir-98_l | 0.017 | −0.441 | −1.200 | 0.759 |
| HS_215_r | 0.001 | 0.927 | 0.167 | 0.760 |
| HS_from_MQbrain_1227_l | 0.043 | 0.258 | −0.508 | 0.766 |
| hsa-mir-668_r | 0.034 | 0.802 | 0.032 | 0.769 |
| hsa-let-7a-2_l | 0.013 | −0.333 | −1.106 | 0.773 |
| HS_from_MQbrain_1169_l | 0.030 | 0.600 | −0.173 | 0.773 |
| hsa-mir-16-1_l | 0.176 | 0.361 | −0.415 | 0.776 |
| HS_from_MQbrain_918_l | 0.030 | 1.730 | 0.948 | 0.782 |
| HS_from_MM_6_r | 0.065 | 0.900 | 0.115 | 0.785 |
| Hsd_33_l | 0.080 | 0.576 | −0.214 | 0.791 |
| HSbrain_760_l | 0.034 | 0.241 | −0.550 | 0.791 |
| hsa-let-7a-3_l | 0.014 | −0.259 | −1.051 | 0.792 |
| HSbrain_636_l | 0.023 | 1.330 | 0.534 | 0.796 |
| Hsd_from_Mmd_533_l | 0.025 | 0.327 | −0.469 | 0.796 |
| HS_from_MQbrain_1183_l | 0.005 | 1.249 | 0.452 | 0.797 |
| hsa-mir-126_r | 0.014 | −0.136 | −0.937 | 0.802 |
| hsa-let-7a-1_l | 0.013 | −0.237 | −1.039 | 0.802 |
| HS_from_MQbrain_1325_r | 0.084 | 1.734 | 0.932 | 0.802 |
| HS_from_MQbrain_1734_l | 0.189 | 2.197 | 1.394 | 0.802 |
| hsa-mir-16-2_l | 0.159 | 0.466 | −0.337 | 0.803 |
| HS_from_MQbrain_1128_l | 0.005 | 0.944 | 0.140 | 0.804 |
| HS_from_MQbrain_1823_r | 0.010 | −0.086 | −0.892 | 0.806 |
| hsa-mir-454_r | 0.016 | −0.337 | −1.148 | 0.811 |
| HS_from_MM_304_l | 0.170 | 1.858 | 1.046 | 0.812 |
| HS_from_MQbrain_366_r | 0.070 | 0.788 | −0.046 | 0.835 |
| HSbrain_916_r | 0.056 | 1.228 | 0.387 | 0.841 |
| HS_from_MQbrain_80_l | 0.108 | 1.137 | 0.295 | 0.843 |
| Hsd_from_Mmd_291_l | 0.135 | 1.914 | 1.066 | 0.848 |
| hsa-mir-633_l | 0.017 | 0.566 | −0.285 | 0.850 |
| HS_291_r | 0.002 | 0.012 | −0.838 | 0.851 |
| HS_192_r | 0.170 | 2.486 | 1.634 | 0.853 |
| HS_21_r | 0.001 | 1.909 | 1.055 | 0.855 |
| HSbrain_916_l | 0.024 | 1.825 | 0.966 | 0.859 |
| HS_from_MQbrain_374_r | 0.108 | 0.701 | −0.162 | 0.863 |
| hsa-mir-148a_r | 0.043 | 0.200 | −0.668 | 0.867 |
| HS_from_MQbrain_965_l | 0.013 | 0.734 | −0.138 | 0.872 |
| HS_from_MQbrain_579_l | 0.056 | 1.113 | 0.240 | 0.872 |
| Hsd_from_Mmd_524_l | 0.135 | −0.469 | −1.342 | 0.873 |
| hsa-mir-181a-2_l | 0.040 | −0.194 | −1.074 | 0.880 |
| HSbrain_1384_r | 0.017 | 0.605 | −0.276 | 0.881 |
| HSbrain_52_r | 0.021 | 0.850 | −0.035 | 0.885 |
| Hsd_from_Mmd_81_l | 0.034 | 0.626 | −0.266 | 0.892 |
| HS_from_MM_1_r | 0.048 | −0.144 | −1.036 | 0.892 |
| Hsd_60_r | 0.119 | 1.274 | 0.371 | 0.902 |
| hsa-mir-130a_r | 0.014 | 0.355 | −0.547 | 0.903 |
| HSbrain_224_r | 0.019 | 0.732 | −0.170 | 0.903 |
| hsa-mir-26a-2_l | 0.183 | 0.327 | −0.583 | 0.910 |
| HS_from_MQbrain_194_l | 0.011 | 1.076 | 0.165 | 0.911 |
| hsa-mir-92b_r | 0.048 | 0.316 | −0.596 | 0.912 |
| HSbrain_253_l | 0.019 | 1.286 | 0.372 | 0.914 |
| HS_from_MQbrain_1462_l | 0.009 | 0.967 | 0.048 | 0.919 |
| hsa-mir-129-1_r | 0.108 | 1.280 | 0.361 | 0.919 |
| Hsd_from_Mmd_298_r | 0.025 | −0.298 | −1.219 | 0.921 |
| HSbrain_1_l | 0.017 | 0.936 | 0.012 | 0.924 |
| HSbrain_337_l | 0.020 | 0.406 | −0.521 | 0.927 |
| HSbrain_19_l | 0.092 | 1.886 | 0.952 | 0.934 |
| HS_from_MQbrain_1958_l | 0.053 | 1.083 | 0.142 | 0.941 |
| HS_from_MM_334_l | 0.004 | 0.627 | −0.324 | 0.951 |
| hsa-mir-26b_l | 0.036 | −0.144 | −1.099 | 0.955 |
| HS_169_l | 0.001 | 1.737 | 0.779 | 0.958 |
| HS_80_l | 0.088 | 2.217 | 1.258 | 0.959 |
| HSbrain_362_r | 0.020 | 1.193 | 0.230 | 0.963 |
| HS_255_l | 0.001 | 1.422 | 0.454 | 0.968 |
| HS_from_MM_264_l | 0.004 | 1.081 | 0.110 | 0.972 |
| HSbrain_500_l | 0.034 | 1.101 | 0.116 | 0.984 |
| Hsd_81_r | 0.065 | −0.348 | −1.336 | 0.989 |
| hsa-let-7g_l | 0.014 | −0.344 | −1.333 | 0.989 |
| hsa-mir-26a-1_l | 0.140 | 0.271 | −0.719 | 0.990 |
| hsa-mir-181a-1_l | 0.015 | −0.200 | −1.192 | 0.992 |
| Hsd_from_Mmd_387_l | 0.025 | 1.078 | 0.082 | 0.996 |
| HSbrain_500_r | 0.021 | 1.019 | 0.021 | 0.998 |
| HS_68_l | 0.103 | −1.388 | −2.391 | 1.003 |
| HS_from_MQbrain_1985_l | 0.030 | 1.111 | 0.108 | 1.004 |
| hsa-mir-20a_l | 0.088 | −0.516 | −1.527 | 1.010 |
| hsa-mir-146a_l | 0.030 | 0.140 | −0.872 | 1.012 |
| hsa-mir-20b_l | 0.058 | −0.713 | −1.732 | 1.019 |
| hsa-let-7f-1_l | 0.014 | −0.167 | −1.192 | 1.025 |
| HS_215_r | 0.001 | 1.669 | 0.641 | 1.028 |
| HS_from_MQbrain_572_l | 0.050 | −0.092 | −1.130 | 1.038 |
| HSbrain_209_l | 0.095 | 1.410 | 0.371 | 1.039 |
| HS_from_MQbrain_1784_r | 0.103 | 1.648 | 0.601 | 1.047 |
| HSbrain_1305_l | 0.017 | 1.470 | 0.421 | 1.049 |
| hsa-let-7f-2_l | 0.030 | −0.121 | −1.173 | 1.052 |
| HS_266_r | 0.046 | 1.371 | 0.315 | 1.056 |
| HSbrain_398_r | 0.020 | 1.061 | 0.004 | 1.057 |
| hsa-mir-200b_r | 0.072 | 1.035 | −0.027 | 1.062 |
| HSbrain_1_r | 0.119 | 1.821 | 0.740 | 1.081 |
| hsa-mir-363_l | 0.072 | 1.689 | 0.587 | 1.102 |
| HS_from_PT_26_r | 0.013 | 1.314 | 0.210 | 1.104 |
| HS_from_MQbrain_1235_l | 0.065 | 1.757 | 0.631 | 1.127 |
| hsa-mir-200c_r | 0.048 | 0.810 | −0.339 | 1.149 |
| HS_291_r | 0.002 | 0.965 | −0.193 | 1.158 |
| HS_from_MQbrain_153_l | 0.153 | 3.559 | 2.394 | 1.165 |
| HS_from_MQbrain_241_l | 0.113 | 2.115 | 0.949 | 1.166 |
| hsa-mir-421_r | 0.016 | 1.426 | 0.252 | 1.175 |
| hsa-mir-29c_r | 0.065 | −0.757 | −1.938 | 1.181 |
| HS_from_MQbrain_79_r | 0.012 | 1.972 | 0.787 | 1.185 |
| hsa-mir-181c_l | 0.015 | 0.066 | −1.122 | 1.188 |
| hsa-mir-145_l | 0.032 | 0.280 | −0.909 | 1.189 |
| HSbrain_237_r | 0.092 | 1.411 | 0.214 | 1.197 |
| HS_from_MQbrain_687_r | 0.077 | 2.596 | 1.397 | 1.199 |
| HS_from_PT_7_r | 0.108 | 2.612 | 1.393 | 1.218 |
| HS_from_MQbrain_1323_l | 0.068 | 1.783 | 0.539 | 1.244 |
| HSbrain_1437_r | 0.017 | 1.047 | −0.202 | 1.248 |
| hsa-mir-21_l | 0.189 | −1.905 | −3.155 | 1.249 |
| hsa-mir-768_r | 0.017 | 1.069 | −0.182 | 1.251 |
| HSbrain_398_l | 0.020 | 0.748 | −0.536 | 1.285 |
| HS_from_MQbrain_2700_r | 0.011 | 1.133 | −0.188 | 1.321 |
| hsa-mir-29a_r | 0.016 | −0.431 | −1.810 | 1.379 |
| hsa-mir-9-2_r | 0.129 | 1.894 | 0.500 | 1.393 |
| HS_from_PT_139_l | 0.029 | 2.178 | 0.775 | 1.403 |
| hsa-mir-27a_r | 0.016 | −0.620 | −2.050 | 1.430 |
| hsa-mir-9-1_r | 0.080 | 1.925 | 0.491 | 1.434 |
| hsa-mir-92-2_r | 0.017 | 0.583 | −0.929 | 1.512 |
| HSbrain_411_r | 0.020 | 0.519 | −0.994 | 1.512 |
| hsa-mir-9-3_r | 0.070 | 1.982 | 0.460 | 1.522 |
| hsa-mir-92-1_r | 0.017 | 0.637 | −0.942 | 1.579 |
| HSbrain_377_r | 0.020 | 1.073 | −0.614 | 1.687 |
| HS_from_PT_114_l | 0.013 | 0.542 | −1.365 | 1.907 |
| HSbrain_377_l | 0.020 | 0.817 | −1.136 | 1.952 |
| HS_from_PT_114_r | 0.013 | 0.790 | −1.277 | 2.067 |

¹Wilcoxon analysis: cut off adj p value <0.2
²upregulation of small RNA when ratio expression >1 downregulation of small RNA when ratio expression <1

TABLE 2c
Small RNAs that are upregulated in colorectal adenomas compared to control samples

| | | |
|---|---|---|
| HS_from_MQbrain_1985_l | HS_from_MQbrain_1235_l | HSbrain_398_l |
| hsa-mir-20a_l | hsa-mir-200c_r | HS_from_MQbrain_2700_r |
| hsa-mir-146a_l | HS_291_r | hsa-mir-29a_r |
| hsa-mir-20b_l | HS_from_MQbrain_153_l | hsa-mir-9-2_r |
| hsa-let-7f-1_l | HS_from_MQbrain_241_l | HS_from_PT_139_l |
| HS_215_r | hsa-mir-421_r | hsa-mir-27a_r |
| HS_from_MQbrain_572_l | hsa-mir-29c_r | hsa-mir-9-1_r |
| HSbrain_209_l | HS_from_MQbrain_79_r | hsa-mir-92-2_r |
| HS_from_MQbrain_1784_r | hsa-mir-181c_l | HSbrain_411_r |
| HSbrain_1305_l | hsa-mir-145_l | hsa-mir-9-3_r |
| hsa-let-7f-2_l | HSbrain_237_r | hsa-mir-92-1_r |
| HS_266_r | HS_from_MQbrain_687_r | HSbrain_377_r |
| HSbrain_398_r | HS_from_PT_7_r | HS_from_PT_114_l |
| hsa-mir-200b_r | HS_from_MQbrain_1323_l | HSbrain_377_l |
| HSbrain_1_r | HSbrain_1437_r | HS_from_PT_114_r |
| hsa-mir-363_l | hsa-mir-21_l | |
| HS_from_PT_26_r | hsa-mir-768_r | |

TABLE 2d
Small RNAs that are downregulated in colorectal adenomas
compared to control samples

HS_101_l
HS_102_l
HS_106_l
HS_106_r
HS_109_l
HS_109_r
HS_11_l
HS_111_l
HS_114_l
HS_115_r
HS_118_l
HS_119_l
HS_121_r
HS_124_l
HS_128_l
HS_128_r
HS_129_l
HS_130_l
HS_131_r
HS_132_l
HS_135_l
HS_141_l
HS_145_r
HS_15_l
HS_150_l
HS_151_r
HS_156_l
HS_157_l
HS_157_r
HS_161_r
HS_164_l
HS_166_r
HS_167_l
HS_168_r
HS_169_l
HS_171_r
HS_172_r
HS_173_l
HS_175_l
HS_176_l
HS_177_l
HS_18_l
HS_180_r
HS_181_l
HS_186_r
HS_189_l
HS_189_r
HS_190_l
HS_190_r
HS_191_l
HS_192_r
HS_197_r
HS_198_l
HS_203_l
HS_203_r
HS_204_l
HS_204_r
HS_21_r
HS_212_l
HS_212_r
HS_213_l
HS_214_r
HS_215_l
HS_215_r
HS_217_l
HS_218_r
HS_219_l
HS_22_r
HS_220_l
HS_223_l
HS_223_r
HS_231_r
HS_232_l
HS_241_r
HS_25_r
HS_255_l
HS_261_l
HS_261_r
HS_262_l
HS_262_r
HS_264_r
HS_265_l
HS_268_l
HS_281_r
HS_281_r
HS_286_r
HS_287_l
HS_288_l
HS_289_l
HS_291_r
HS_292_l
HS_304_r
HS_306_r
HS_307_r
HS_33_l
HS_36_l
HS_38_r
HS_4_r
HS_41_r
HS_49_r
HS_51_r
HS_53_r
HS_56_r
HS_57_l
HS_59_l
HS_61_l
HS_62_l
HS_66_r
HS_71_l
HS_71_r
HS_73_r
HS_74_r
HS_76_r
HS_77_l
HS_77_r
HS_78_r
HS_79_l
HS_80_l
HS_81_r
HS_83_r
HS_85_l
HS_9_r
HS_93_r
HS_95_r
HS_97_r
HS_98_l
HS_98_l
HS_98_r
HS_from_MM_1_l
HS_from_MM_1_r
HS_from_MM_103_l
HS_from_MM_109_l
HS_from_MM_115_l
HS_from_MM_118_l
HS_from_MM_118_r
HS_from_MM_119_r
HS_from_MM_121_l
HS_from_MM_123_l
HS_from_MM_124_l
HS_from_MM_124_r
HS_from_MM_131_l
HS_from_MM_135_l
HS_from_MM_145_r
HS_from_MM_149_r
HS_from_MM_150_r
HS_from_MM_157_l
HS_from_MM_160_l
HS_from_MM_163_l
HS_from_MM_165_l
HS_from_MM_169_r
HS_from_MM_174_l
HS_from_MM_178_r
HS_from_MM_18_l
HS_from_MM_182_r
HS_from_MM_184_r
HS_from_MM_185_r
HS_from_MM_194_l
HS_from_MM_195_r
HS_from_MM_202_l
HS_from_MM_218_l
HS_from_MM_219_l
HS_from_MM_229_r
HS_from_MM_232_r
HS_from_MM_237_l
HS_from_MM_241_l
HS_from_MM_251_r
HS_from_MM_264_l
HS_from_MM_276_l
HS_from_MM_289_l
HS_from_MM_289_r
HS_from_MM_295_l
HS_from_MM_295_r
HS_from_MM_298_l
HS_from_MM_298_r
HS_from_MM_304_l
HS_from_MM_304_r
HS_from_MM_309_r
HS_from_MM_313_l
HS_from_MM_319_l
HS_from_MM_32_r
HS_from_MM_331_r
HS_from_MM_334_l
HS_from_MM_339_r
HS_from_MM_34_l
HS_from_MM_340_r
HS_from_MM_342_l
HS_from_MM_342_r
HS_from_MM_346_r
HS_from_MM_347_l
HS_from_MM_35_l
HS_from_MM_353_l
HS_from_MM_354_r
HS_from_MM_359_l
HS_from_MM_39_l
HS_from_MM_47_l
HS_from_MM_48_l
HS_from_MM_49_l
HS_from_MM_57_r
HS_from_MM_6_l
HS_from_MM_6_r
HS_from_MM_63_l
HS_from_MM_72_r
HS_from_MM_79_l
HS_from_MM_84_l
HS_from_MM_84_r
HS_from_MM_86_l
HS_from_MM_87_l
HS_from_MM_89_l
HS_from_MM_92_r
HS_from_MQbrain_100_l
HS_from_MQbrain_1006_l
HS_from_MQbrain_1018_l
HS_from_MQbrain_1028_l
HS_from_MQbrain_1030_l
HS_from_MQbrain_1031_l
HS_from_MQbrain_1036_r
HS_from_MQbrain_1037_r
HS_from_MQbrain_1042_r
HS_from_MQbrain_1043_r
HS_from_MQbrain_105_l
HS_from_MQbrain_105_l
HS_from_MQbrain_1051_r
HS_from_MQbrain_1056_l
HS_from_MQbrain_1064_r
HS_from_MQbrain_1070_r
HS_from_MQbrain_1072_r
HS_from_MQbrain_1073_r
HS_from_MQbrain_1075_l
HS_from_MQbrain_1080_l
HS_from_MQbrain_1081_r
HS_from_MQbrain_1082_l
HS_from_MQbrain_1085_l
HS_from_MQbrain_1087_l
HS_from_MQbrain_1090_r
HS_from_MQbrain_1097_r
HS_from_MQbrain_1099_r
HS_from_MQbrain_1100_l
HS_from_MQbrain_1102_l
HS_from_MQbrain_1103_l
HS_from_MQbrain_1109_l
HS_from_MQbrain_1110_l
HS_from_MQbrain_1119_l
HS_from_MQbrain_1119_l
HS_from_MQbrain_1126_l
HS_from_MQbrain_1127_r
HS_from_MQbrain_1128_l
HS_from_MQbrain_113_r
HS_from_MQbrain_1137_l
HS_from_MQbrain_1139_r
HS_from_MQbrain_114_r
HS_from_MQbrain_1143_r
HS_from_MQbrain_1164_l
HS_from_MQbrain_1169_l
HS_from_MQbrain_1183_l
HS_from_MQbrain_1184_r
HS_from_MQbrain_1199_l
HS_from_MQbrain_120_l
HS_from_MQbrain_1201_r
HS_from_MQbrain_1204_l
HS_from_MQbrain_1204_r
HS_from_MQbrain_1208_l
HS_from_MQbrain_1212_l
HS_from_MQbrain_1213_l
HS_from_MQbrain_1215_l
HS_from_MQbrain_1219_l
HS_from_MQbrain_1219_r
HS_from_MQbrain_1222_r
HS_from_MQbrain_1223_l
HS_from_MQbrain_1224_r
HS_from_MQbrain_1227_l
HS_from_MQbrain_1227_l
HS_from_MQbrain_1228_r
HS_from_MQbrain_1234_l
HS_from_MQbrain_1239_l
HS_from_MQbrain_1241_r
HS_from_MQbrain_1245_l
HS_from_MQbrain_1246_l
HS_from_MQbrain_1250_r
HS_from_MQbrain_1253_r
HS_from_MQbrain_1260_r
HS_from_MQbrain_1267_r
HS_from_MQbrain_1268_l
HS_from_MQbrain_128_r
HS_from_MQbrain_1280_l
HS_from_MQbrain_1283_l
HS_from_MQbrain_129_r
HS_from_MQbrain_1292_r
HS_from_MQbrain_1297_l
HS_from_MQbrain_1300_l
HS_from_MQbrain_1300_r
HS_from_MQbrain_1304_r
HS_from_MQbrain_131_r
HS_from_MQbrain_1311_r
HS_from_MQbrain_1314_l
HS_from_MQbrain_1315_l
HS_from_MQbrain_1317_r
HS_from_MQbrain_1318_l
HS_from_MQbrain_1325_r
HS_from_MQbrain_1334_r
HS_from_MQbrain_1340_l
HS_from_MQbrain_1349_l
HS_from_MQbrain_135_l
HS_from_MQbrain_1353_l
HS_from_MQbrain_1354_l
HS_from_MQbrain_1355_r
HS_from_MQbrain_1363_l
HS_from_MQbrain_1366_l
HS_from_MQbrain_137_r
HS_from_MQbrain_1374_l
HS_from_MQbrain_1376_l
HS_from_MQbrain_1376_r
HS_from_MQbrain_1377_l
HS_from_MQbrain_1377_r
HS_from_MQbrain_1379_r
HS_from_MQbrain_1380_r
HS_from_MQbrain_1381_r
HS_from_MQbrain_1382_l
HS_from_MQbrain_1384_r
HS_from_MQbrain_1388_l
HS_from_MQbrain_1392_r
HS_from_MQbrain_1393_r
HS_from_MQbrain_1395_r
HS_from_MQbrain_140_l
HS_from_MQbrain_1402_r
HS_from_MQbrain_1404_r
HS_from_MQbrain_1410_l
HS_from_MQbrain_1412_l
HS_from_MQbrain_1414_r
HS_from_MQbrain_1417_l
HS_from_MQbrain_1420_l
HS_from_MQbrain_1424_r
HS_from_MQbrain_1426_l
HS_from_MQbrain_1427_l
HS_from_MQbrain_1437_l
HS_from_MQbrain_1442_l
HS_from_MQbrain_1443_r
HS_from_MQbrain_1444_l
HS_from_MQbrain_1444_l
HS_from_MQbrain_1445_l
HS_from_MQbrain_1446_l
HS_from_MQbrain_1450_r
HS_from_MQbrain_1456_l
HS_from_MQbrain_1459_l
HS_from_MQbrain_1462_l
HS_from_MQbrain_1462_r
HS_from_MQbrain_1469_r
HS_from_MQbrain_147_l
HS_from_MQbrain_1478_l
HS_from_MQbrain_1484_l
HS_from_MQbrain_1485_l
HS_from_MQbrain_1485_r
HS_from_MQbrain_1487_r
HS_from_MQbrain_1493_r
HS_from_MQbrain_1494_l
HS_from_MQbrain_1497_l
HS_from_MQbrain_15_r
HS_from_MQbrain_1500_l
HS_from_MQbrain_1501_r
HS_from_MQbrain_1502_l
HS_from_MQbrain_1509_r
HS_from_MQbrain_1513_r
HS_from_MQbrain_1520_l
HS_from_MQbrain_1520_r
HS_from_MQbrain_1522_r
HS_from_MQbrain_1523_r
HS_from_MQbrain_1535_r
HS_from_MQbrain_154_l
HS_from_MQbrain_154_r
HS_from_MQbrain_1545_r
HS_from_MQbrain_155_l
HS_from_MQbrain_1561_l
HS_from_MQbrain_1561_r
HS_from_MQbrain_1565_l
HS_from_MQbrain_1565_r
HS_from_MQbrain_1566_r
HS_from_MQbrain_1574_l
HS_from_MQbrain_1576_r
HS_from_MQbrain_1590_l
HS_from_MQbrain_1592_l
HS_from_MQbrain_1593_r
HS_from_MQbrain_1596_r
HS_from_MQbrain_1602_l
HS_from_MQbrain_161_r
HS_from_MQbrain_1610_r
HS_from_MQbrain_162_r
HS_from_MQbrain_1620_l
HS_from_MQbrain_1624_l
HS_from_MQbrain_1625_r
HS_from_MQbrain_1626_r
HS_from_MQbrain_1627_l
HS_from_MQbrain_1628_r
HS_from_MQbrain_163_l
HS_from_MQbrain_1630_l
HS_from_MQbrain_1632_r
HS_from_MQbrain_1633_r
HS_from_MQbrain_1637_l
HS_from_MQbrain_1637_r
HS_from_MQbrain_1640_l
HS_from_MQbrain_1642_l
HS_from_MQbrain_1648_r
HS_from_MQbrain_1657_r
HS_from_MQbrain_1658_l
HS_from_MQbrain_1658_r
HS_from_MQbrain_1665_r
HS_from_MQbrain_1671_l
HS_from_MQbrain_1674_l
HS_from_MQbrain_1675_r
HS_from_MQbrain_1678_r
HS_from_MQbrain_1684_l
HS_from_MQbrain_1684_r
HS_from_MQbrain_1686_l
HS_from_MQbrain_1687_l
HS_from_MQbrain_1690_l
HS_from_MQbrain_1692_l
HS_from_MQbrain_1695_l
HS_from_MQbrain_1695_r
HS_from_MQbrain_1696_l
HS_from_MQbrain_1700_l
HS_from_MQbrain_1700_r
HS_from_MQbrain_1703_r
HS_from_MQbrain_1706_l
HS_from_MQbrain_1712_r
HS_from_MQbrain_1717_r
HS_from_MQbrain_1721_l
HS_from_MQbrain_1722_l
HS_from_MQbrain_1724_l
HS_from_MQbrain_1726_l
HS_from_MQbrain_1734_l
HS_from_MQbrain_1744_r
HS_from_MQbrain_1745_r
HS_from_MQbrain_1747_r
HS_from_MQbrain_1748_r
HS_from_MQbrain_1750_r
HS_from_MQbrain_1752_l
HS_from_MQbrain_1753_r
HS_from_MQbrain_1758_r
HS_from_MQbrain_1759_l
HS_from_MQbrain_1760_r
HS_from_MQbrain_1761_r
HS_from_MQbrain_1771_l
HS_from_MQbrain_1771_r
HS_from_MQbrain_1773_l
HS_from_MQbrain_1773_l
HS_from_MQbrain_1776_l
HS_from_MQbrain_1777_l
HS_from_MQbrain_178_r
HS_from_MQbrain_1781_l
HS_from_MQbrain_1783_r
HS_from_MQbrain_1792_r
HS_from_MQbrain_1793_l
HS_from_MQbrain_1794_l
HS_from_MQbrain_1794_r
HS_from_MQbrain_1804_r
HS_from_MQbrain_1806_l
HS_from_MQbrain_1807_l
HS_from_MQbrain_1808_r
HS_from_MQbrain_1817_l
HS_from_MQbrain_1823_r
HS_from_MQbrain_1831_r
HS_from_MQbrain_1832_l
HS_from_MQbrain_1832_r
HS_from_MQbrain_1834_r
HS_from_MQbrain_1839_l
HS_from_MQbrain_1840_l
HS_from_MQbrain_1841_r
HS_from_MQbrain_1844_r
HS_from_MQbrain_1846_r
HS_from_MQbrain_1855_l
HS_from_MQbrain_1862_l
HS_from_MQbrain_1863_l
HS_from_MQbrain_1877_l
HS_from_MQbrain_188_l
HS_from_MQbrain_1883_l
HS_from_MQbrain_1883_r
HS_from_MQbrain_1884_r
HS_from_MQbrain_1885_r
HS_from_MQbrain_1889_l
HS_from_MQbrain_1893_r
HS_from_MQbrain_1896_r
HS_from_MQbrain_191_l
HS_from_MQbrain_194_l
HS_from_MQbrain_1958_l
HS_from_MQbrain_20_r
HS_from_MQbrain_207_l
HS_from_MQbrain_2087_r
HS_from_MQbrain_209_r
HS_from_MQbrain_212_r
HS_from_MQbrain_22_r
HS_from_MQbrain_221_r
HS_from_MQbrain_224_l
HS_from_MQbrain_234_r
HS_from_MQbrain_235_r
HS_from_MQbrain_2380_r
HS_from_MQbrain_239_r
HS_from_MQbrain_251_r
HS_from_MQbrain_253_r
HS_from_MQbrain_255_l
HS_from_MQbrain_260_r
HS_from_MQbrain_266_r
HS_from_MQbrain_2670_r
HS_from_MQbrain_2699_l
HS_from_MQbrain_281_l
HS_from_MQbrain_281_r
HS_from_MQbrain_282_l
HS_from_MQbrain_283_r
HS_from_MQbrain_288_r
HS_from_MQbrain_29_r
HS_from_MQbrain_291_l
HS_from_MQbrain_301_l
HS_from_MQbrain_314_l
HS_from_MQbrain_315_l
HS_from_MQbrain_330_r
HS_from_MQbrain_338_l
HS_from_MQbrain_340_l
HS_from_MQbrain_343_l
HS_from_MQbrain_349_l
HS_from_MQbrain_350_l
HS_from_MQbrain_354_r
HS_from_MQbrain_355_r
HS_from_MQbrain_36_r
HS_from_MQbrain_361_l
HS_from_MQbrain_365_l
HS_from_MQbrain_365_r
HS_from_MQbrain_366_r
HS_from_MQbrain_37_r
HS_from_MQbrain_374_r
HS_from_MQbrain_375_l
HS_from_MQbrain_38_r
HS_from_MQbrain_382_l
HS_from_MQbrain_382_r
HS_from_MQbrain_383_l
HS_from_MQbrain_384_r
HS_from_MQbrain_389_l
HS_from_MQbrain_391_r
HS_from_MQbrain_392_r
HS_from_MQbrain_406_l
HS_from_MQbrain_406_r
HS_from_MQbrain_412_l
HS_from_MQbrain_414_l
HS_from_MQbrain_420_l
HS_from_MQbrain_43_l
HS_from_MQbrain_433_l
HS_from_MQbrain_435_l
HS_from_MQbrain_435_r
HS_from_MQbrain_436_r
HS_from_MQbrain_437_l
HS_from_MQbrain_441_l
HS_from_MQbrain_444_r
HS_from_MQbrain_449_l
HS_from_MQbrain_458_l
HS_from_MQbrain_461_l
HS_from_MQbrain_466_l
HS_from_MQbrain_467_l
HS_from_MQbrain_468_l
HS_from_MQbrain_468_r
HS_from_MQbrain_470_l
HS_from_MQbrain_472_r
HS_from_MQbrain_48_r
HS_from_MQbrain_482_l
HS_from_MQbrain_485_l
HS_from_MQbrain_487_l
HS_from_MQbrain_487_r
HS_from_MQbrain_490_r
HS_from_MQbrain_498_r
HS_from_MQbrain_502_r
HS_from_MQbrain_509_r
HS_from_MQbrain_520_l
HS_from_MQbrain_524_l
HS_from_MQbrain_529_r
HS_from_MQbrain_539_r
HS_from_MQbrain_540_l
HS_from_MQbrain_542_r
HS_from_MQbrain_546_l
HS_from_MQbrain_549_l
HS_from_MQbrain_549_r
HS_from_MQbrain_55_l
HS_from_MQbrain_550_l
HS_from_MQbrain_555_r
HS_from_MQbrain_559_r
HS_from_MQbrain_56_l
HS_from_MQbrain_56_r
HS_from_MQbrain_560_r
HS_from_MQbrain_566_l
HS_from_MQbrain_566_r
HS_from_MQbrain_567_r
HS_from_MQbrain_574_l
HS_from_MQbrain_576_l
HS_from_MQbrain_579_l
HS_from_MQbrain_590_l
HS_from_MQbrain_592_l
HS_from_MQbrain_594_r
HS_from_MQbrain_596_r
HS_from_MQbrain_597_l
HS_from_MQbrain_597_r
HS_from_MQbrain_6_l
HS_from_MQbrain_6_r
HS_from_MQbrain_600_l
HS_from_MQbrain_603_r
HS_from_MQbrain_604_l
HS_from_MQbrain_609_l
HS_from_MQbrain_619_r
HS_from_MQbrain_625_l
HS_from_MQbrain_628_l
HS_from_MQbrain_629_l
HS_from_MQbrain_629_r
HS_from_MQbrain_631_l
HS_from_MQbrain_634_l
HS_from_MQbrain_635_l
HS_from_MQbrain_641_r
HS_from_MQbrain_6481
HS_from_MQbrain_65_l
HS_from_MQbrain_650_r
HS_from_MQbrain_655_l
HS_from_MQbrain_664_l
HS_from_MQbrain_664_r
HS_from_MQbrain_669_r
HS_from_MQbrain_67_l
HS_from_MQbrain_67_r
HS_from_MQbrain_671_r
HS_from_MQbrain_675_l
HS_from_MQbrain_675_r
HS_from_MQbrain_676_r
HS_from_MQbrain_68_r
HS_from_MQbrain_684_r
HS_from_MQbrain_686_r
HS_from_MQbrain_689_l
HS_from_MQbrain_691_l
HS_from_MQbrain_693_r
HS_from_MQbrain_694_r
HS_from_MQbrain_697_l
HS_from_MQbrain_698_r
HS_from_MQbrain_701_l
HS_from_MQbrain_707_l
HS_from_MQbrain_71_r
HS_from_MQbrain_715_l
HS_from_MQbrain_723_l
HS_from_MQbrain_723_r
HS_from_MQbrain_732_r
HS_from_MQbrain_734_l
HS_from_MQbrain_736_r
HS_from_MQbrain_739_l
HS_from_MQbrain_74_r
HS_from_MQbrain_745_r
HS_from_MQbrain_750_r
HS_from_MQbrain_751_l
HS_from_MQbrain_755_l
HS_from_MQbrain_768_l
HS_from_MQbrain_769_r
HS_from_MQbrain_770_r
HS_from_MQbrain_774_r
HS_from_MQbrain_779_l
HS_from_MQbrain_782_r
HS_from_MQbrain_789_l
HS_from_MQbrain_791_r
HS_from_MQbrain_796_l
HS_from_MQbrain_80_l
HS_from_MQbrain_800_r
HS_from_MQbrain_804_r
HS_from_MQbrain_806_l
HS_from_MQbrain_810_l
HS_from_MQbrain_814_l
HS_from_MQbrain_824_l
HS_from_MQbrain_824_r
HS_from_MQbrain_825_r
HS_from_MQbrain_828_r
HS_from_MQbrain_839_r
HS_from_MQbrain_84_r
HS_from_MQbrain_841_l
HS_from_MQbrain_842_l
HS_from_MQbrain_843_r
HS_from_MQbrain_848_l
HS_from_MQbrain_850_l
HS_from_MQbrain_854_r
HS_from_MQbrain_855_r
HS_from_MQbrain_868_l
HS_from_MQbrain_873_r
HS_from_MQbrain_88_l
HS_from_MQbrain_881_l
HS_from_MQbrain_882_l
HS_from_MQbrain_883_l
HS_from_MQbrain_883_r
HS_from_MQbrain_89_l
HS_from_MQbrain_897_l
HS_from_MQbrain_900_r
HS_from_MQbrain_901_l
HS_from_MQbrain_901_r
HS_from_MQbrain_906_r
HS_from_MQbrain_912_l
HS_from_MQbrain_917_r
HS_from_MQbrain_918_l
HS_from_MQbrain_92_l
HS_from_MQbrain_921_l
HS_from_MQbrain_930_r
HS_from_MQbrain_932_l
HS_from_MQbrain_936_r
HS_from_MQbrain_940_r
HS_from_MQbrain_941_r
HS_from_MQbrain_943_l
HS_from_MQbrain_948_r
HS_from_MQbrain_951_l
HS_from_MQbrain_951_l
HS_from_MQbrain_965_l
HS_from_MQbrain_967_l
HS_from_MQbrain_968_l
HS_from_MQbrain_969_l
HS_from_MQbrain_969_r
HS_from_MQbrain_97_l
HS_from_MQbrain_97_r
HS_from_MQbrain_974_l
HS_from_MQbrain_984_l
HS_from_MQbrain_99_r
HS_from_MQbrain_991_l
HS_from_MQbrain_991_r
HS_from_MQbrain_996_l
HS_from_MQbrain_997_l
HS_from_MQbrain_999_l
HS_from_MQbrain_999_r
HS_from_PT_101_r
HS_from_PT_102_l
HS_from_PT_103_l
HS_from_PT_104_l
HS_from_PT_112_l
HS_from_PT_121_r
HS_from_PT_126_l
HS_from_PT_130_r
HS_from_PT_136_l
HS_from_PT_146_r
HS_from_PT_148_l
HS_from_PT_155_l
HS_from_PT_158_l
HS_from_PT_161_l
HS_from_PT_170_l
HS_from_PT_170_r
HS_from_PT_178_l
HS_from_PT_187_l
HS_from_PT_187_r
HS_from_PT_188_l
HS_from_PT_190_r
HS_from_PT_194_r
HS_from_PT_196_l
HS_from_PT_196_r
HS_from_PT_20_r
HS_from_PT_201_l
HS_from_PT_201_r
HS_from_PT_204_r
HS_from_PT_207_l
HS_from_PT_21_r
HS_from_PT_219_l
HS_from_PT_22_l
HS_from_PT_22_l
HS_from_PT_220_l
HS_from_PT_234_l
HS_from_PT_235_l
HS_from_PT_237_l
HS_from_PT_237_r
HS_from_PT_238_l
HS_from_PT_242_l
HS_from_PT_247_l
HS_from_PT_250_l
HS_from_PT_250_r
HS_from_PT_254_r
HS_from_PT_255_l
HS_from_PT_257_r
HS_from_PT_27_l
HS_from_PT_27_r
HS_from_PT_271_l
HS_from_PT_271_r
HS_from_PT_280_l
HS_from_PT_283_r
HS_from_PT_284_r
HS_from_PT_295_r
HS_from_PT_299_l
HS_from_PT_299_r
HS_from_PT_3_l
HS_from_PT_308_l
HS_from_PT_313_r
HS_from_PT_32_r
HS_from_PT_321_r
HS_from_PT_328_l
HS_from_PT_339_l
HS_from_PT_339_r
HS_from_PT_340_l
HS_from_PT_36_l
HS_from_PT_38_l
HS_from_PT_40_r
HS_from_PT_41_r
HS_from_PT_43_l
HS_from_PT_48_l
HS_from_PT_50_r
HS_from_PT_63_r
HS_from_PT_70_r
HS_from_PT_73_l
HS_from_PT_74_r
HS_from_PT_91_r
HS_from_PT_92_l
HS_from_PT_95_l
HS_from_PT_95_r
HS_from_PT_99_r
hsa-let-7a-1_l
hsa-let-7a-2_l
hsa-let-7a-2_r
hsa-let-7a-3_l
hsa-let-7c_l
hsa-let-7c_r
hsa-let-7d_l
hsa-let-7f-1_r
hsa-let-7g_l
hsa-mir-101-1_r
hsa-mir-106b_r
hsa-mir-10a_r
hsa-mir-1-1_l
hsa-mir-1-2_l
hsa-mir-124a-1_l
hsa-mir-124a-1_r
hsa-mir-124a-2_r
hsa-mir-124a-3_r
hsa-mir-125a_l
hsa-mir-125a_r
hsa-mir-125b-1_r
hsa-mir-126_r
hsa-mir-129-1_r
hsa-mir-130a_r
hsa-mir-133a-1_r
hsa-mir-133a-2_r
hsa-mir-134_r
hsa-mir-135a-1_r
hsa-mir-135a-2_r
hsa-mir-137_l
hsa-mir-139_l
hsa-mir-140_l
hsa-mir-141_r
hsa-mir-144_r
hsa-mir-148a_l
hsa-mir-148a_r
hsa-mir-148b_r
hsa-mir-151_l
hsa-mir-155_l
hsa-mir-16-1_l
hsa-mir-16-2_l
hsa-mir-17_l
hsa-mir-181a-1_l
hsa-mir-181a-1_r
hsa-mir-181a-2_l
hsa-mir-183_r
hsa-mir-185_r
hsa-mir-188_l
hsa-mir-190_r
hsa-mir-192_r
hsa-mir-193a_l
hsa-mir-196a-2_r
hsa-mir-196b_r
hsa-mir-197_l
hsa-mir-199a-1_r
hsa-mir-199a-2_r
hsa-mir-203_r
hsa-mir-204_r
hsa-mir-211_r
hsa-mir-219-1_r
hsa-mir-224_l
hsa-mir-224_r
hsa-mir-23a_l
hsa-mir-26a-1_l
hsa-mir-26a-2_l
hsa-mir-26b_l
hsa-mir-27b_r
hsa-mir-296_l
hsa-mir-296_r
hsa-mir-299_l
hsa-mir-299_r
hsa-mir-302d_l
hsa-mir-30b_l
hsa-mir-30c-1_l
hsa-mir-30c-2_l
hsa-mir-30d_l
hsa-mir-31_r
hsa-mir-323_l
hsa-mir-324_l
hsa-mir-324_r
hsa-mir-325_l
hsa-mir-329-1_r
hsa-mir-329-2_r
hsa-mir-331_l
hsa-mir-331_r
hsa-mir-33b_l
hsa-mir-372_r
hsa-mir-373_r
hsa-mir-375_l
hsa-mir-377_l
hsa-mir-378_r
hsa-mir-379_l
hsa-mir-383_l
hsa-mir-383_r
hsa-mir-409_r
hsa-mir-412_l
hsa-mir-412_r
hsa-mir-422a_l
hsa-mir-423_r
hsa-mir-432_l
hsa-mir-432_r
hsa-mir-449b_r
hsa-mir-453_l
hsa-mir-454_l
hsa-mir-454_r
hsa-mir-483_l
hsa-mir-497_r
hsa-mir-500_l
hsa-mir-502_l
hsa-mir-506_l
hsa-mir-506_r
hsa-mir-507_l
hsa-mir-513-1_l
hsa-mir-513-2_l
hsa-mir-514-1_r
hsa-mir-514-3_r
hsa-mir-516-3_l
hsa-mir-516-4_l
hsa-mir-518b_r
hsa-mir-518c_l
hsa-mir-518f_r
hsa-mir-520b_r
hsa-mir-520f_r
hsa-mir-521-1_l
hsa-mir-521-1_r
hsa-mir-521-2_r
hsa-mir-524_l
hsa-mir-525_l
hsa-mir-526a-1_r
hsa-mir-526b_r
hsa-mir-532_l
hsa-mir-550-1_r
hsa-mir-550-2_r
hsa-mir-554_l
hsa-mir-568_r
hsa-mir-570_l
hsa-mir-571_r
hsa-mir-573_l
hsa-mir-584_r
hsa-mir-587_r
hsa-mir-588_r
hsa-mir-591_r
hsa-mir-598_l
hsa-mir-599_r
hsa-mir-604_l
hsa-mir-609_r
hsa-mir-616_l
hsa-mir-616_r
hsa-mir-617_l
hsa-mir-619_l
hsa-mir-622_l
hsa-mir-622_r
hsa-mir-624_l
hsa-mir-625_r
hsa-mir-628_r
hsa-mir-630_r
hsa-mir-633_l
hsa-mir-633_r
hsa-mir-634_l
hsa-mir-635_l
hsa-mir-640_l
hsa-mir-641_l
hsa-mir-641_r
hsa-mir-644_r
hsa-mir-646_r
hsa-mir-647_r
hsa-mir-648_l
hsa-mir-652_l
hsa-mir-652_r
hsa-mir-653_l
hsa-mir-655_l
hsa-mir-659_l
hsa-mir-661_r
hsa-mir-662_l
hsa-mir-668_l
hsa-mir-668_r
hsa-mir-765_r
hsa-mir-767_r
hsa-mir-770_r
hsa-mir-92-1_l
hsa-mir-92b_r
hsa-mir-93_l
hsa-mir-95_r
hsa-mir-98_l
hsa-mir-98_r
hsa-mir-99a_r
HSbrain_1_l
HSbrain_102_r
HSbrain_106_r
HSbrain_107_r
HSbrain_1076_l
HSbrain_1076_r
HSbrain_108_l
HSbrain_109_l
HSbrain_113_r
HSbrain_114_r
HSbrain_116_l
HSbrain_12_l
HSbrain_120_r
HSbrain_121_l
HSbrain_122_l
HSbrain_123_l
HSbrain_124_l
HSbrain_126_r
HSbrain_1298_r
HSbrain_130_l
HSbrain_130_r
HSbrain_131_l
HSbrain_1325_r
HSbrain_1337_l
HSbrain_1338_r
HSbrain_135_l
HSbrain_1360_l
HSbrain_1362_l
HSbrain_1363_l
HSbrain_1364_l
HSbrain_1377_l
HSbrain_1384_r
HSbrain_139_l
HSbrain_139_r
HSbrain_1406_l
HSbrain_1410_l
HSbrain_1413_l
HSbrain_1418_r
HSbrain_1423_l
HSbrain_1425_l
HSbrain_1425_r
HSbrain_143_l
HSbrain_143_r
HSbrain_144_r
HSbrain_145_l
HSbrain_148_r
HSbrain_151_r
HSbrain_152_l
HSbrain_153_r
HSbrain_155_r
HSbrain_156_l
HSbrain_157_l
HSbrain_158_l
HSbrain_159_l
HSbrain_165_l
HSbrain_170_r
HSbrain_171_l
HSbrain_171_l
HSbrain_172_l
HSbrain_177_l
HSbrain_180_l
HSbrain_19_l
HSbrain_198_l
HSbrain_2_l
HSbrain_204_r
HSbrain_210_l
HSbrain_212_l
HSbrain_214_l
HSbrain_216_l
HSbrain_216_r
HSbrain_22_l
HSbrain_220_r
HSbrain_222_r
HSbrain_224_r
HSbrain_225_r
HSbrain_227_r
HSbrain_231_l
HSbrain_237_l
HSbrain_239_l
HSbrain_240_l
HSbrain_242_l
HSbrain_243_l
HSbrain_244_l
HSbrain_245_r
HSbrain_248_l
HSbrain_25_r
HSbrain_250_r
HSbrain_253_l
HSbrain_259_r
HSbrain_261_l
HSbrain_266_l
HSbrain_266_r
HSbrain_267_l
HSbrain_267_r
HSbrain_268_r
HSbrain_270_l
HSbrain_271_l
HSbrain_272_l
HSbrain_275_r
HSbrain_276_l
HSbrain_280_l
HSbrain_284_l
HSbrain_286_l
HSbrain_295_r
HSbrain_296_l
HSbrain_299_r
HSbrain_300_l
HSbrain_301_l
HSbrain_304_l
HSbrain_304_r
HSbrain_305_l
HSbrain_305_r
HSbrain_306_r
HSbrain_316_l
HSbrain_317_l
HSbrain_318_l
HSbrain_319_l
HSbrain_32_l
HSbrain_32_r
HSbrain_320_l
HSbrain_321_r
HSbrain_325_r
HSbrain_328_l
HSbrain_330_l
HSbrain_334_r
HSbrain_337_l
HSbrain_343_l
HSbrain_344_l
HSbrain_347_l
HSbrain_347_r
HSbrain_35_r
HSbrain_359_l
HSbrain_359_r
HSbrain_361_l
HSbrain_362_r
HSbrain_364_r
HSbrain_370_r
HSbrain_371_r
HSbrain_374_r
HSbrain_376_l
HSbrain_383_l
HSbrain_384_l
HSbrain_388_r
HSbrain_389_l
HSbrain_390_l
HSbrain_396_r
HSbrain_399_l
HSbrain_4_l
HSbrain_402_l
HSbrain_403_l
HSbrain_408_r
HSbrain_409_r
HSbrain_41_l
HSbrain_41_r
HSbrain_412_l
HSbrain_413_l
HSbrain_415_l
HSbrain_419_l
HSbrain_420_r
HSbrain_425_l
HSbrain_426_r
HSbrain_43_r
HSbrain_431_r
HSbrain_433_r
HSbrain_437_r
HSbrain_438_r
HSbrain_442_r
HSbrain_446_l
HSbrain_446_r
HSbrain_448_l
HSbrain_452_l
HSbrain_453_r
HSbrain_455_r
HSbrain_456_r
HSbrain_458_l
HSbrain_458_r
HSbrain_463_r
HSbrain_467_l
HSbrain_469_l
HSbrain_47_r
HSbrain_471_r
HSbrain_472_l
HSbrain_473_l
HSbrain_476_r
HSbrain_478_r
HSbrain_48_r
HSbrain_480_r
HSbrain_481_r
HSbrain_482_l
HSbrain_483_l
HSbrain_483_r
HSbrain_485_r
HSbrain_491_l
HSbrain_495_r
HSbrain_496_r
HSbrain_50_l
HSbrain_50_r
HSbrain_500_l
HSbrain_500_r
HSbrain_503_r
HSbrain_504_r
HSbrain_506_l
HSbrain_506_r
HSbrain_507_l
HSbrain_509_l
HSbrain_52_r
HSbrain_520_l
HSbrain_522_r
HSbrain_527_r
HSbrain_53_l
HSbrain_530_l
HSbrain_536_r
HSbrain_540_l
HSbrain_540_r
HSbrain_541_r
HSbrain_543_l
HSbrain_543_r
HSbrain_545_r
HSbrain_546_r
HSbrain_548_l
HSbrain_55_l
HSbrain_553_l
HSbrain_557_r
HSbrain_56_l
HSbrain_568_r
HSbrain_570_l
HSbrain_570_r
HSbrain_571_l
HSbrain_572_r
HSbrain_578_l
HSbrain_580_r
HSbrain_583_l
HSbrain_585_l
HSbrain_590_l
HSbrain_590_r
HSbrain_596_r
HSbrain_597_r
HSbrain_598_l
HSbrain_598_r
HSbrain_599_r
HSbrain_6_r
HSbrain_604_r
HSbrain_610_r
HSbrain_615_l
HSbrain_616_r
HSbrain_617_l
HSbrain_619_r
HSbrain_624_l
HSbrain_626_l
HSbrain_627_r
HSbrain_629_l
HSbrain_635_l
HSbrain_636_l
HSbrain_637_l
HSbrain_641_r
HSbrain_642_r
HSbrain_643_l
HSbrain_652_l
HSbrain_653_l
HSbrain_656_l
HSbrain_657_l
HSbrain_658_l
HSbrain_661_l
HSbrain_663_l
HSbrain_663_r
HSbrain_664_r
HSbrain_669_r
HSbrain_671_r
HSbrain_673_l
HSbrain_673_r
HSbrain_677_l
HSbrain_680_l
HSbrain_680_r
HSbrain_684_r
HSbrain_687_r
HSbrain_690_r
HSbrain_691_l
HSbrain_691_l
HSbrain_695_l
HSbrain_695_r
HSbrain_696_r
HSbrain_70_r
HSbrain_700_r
HSbrain_701_l
HSbrain_703_l
HSbrain_703_r
HSbrain_704_l
HSbrain_705_r
HSbrain_706_l
HSbrain_707_r
HSbrain_708_l
HSbrain_709_l
HSbrain_711_l
HSbrain_711_r
HSbrain_713_l
HSbrain_713_r
HSbrain_714_l
HSbrain_715_l
HSbrain_716_l
HSbrain_717_l
HSbrain_717_r
HSbrain_719_l
HSbrain_72_l
HSbrain_720_l
HSbrain_721_r
HSbrain_722_l
HSbrain_722_r
HSbrain_723_l
HSbrain_724_l
HSbrain_726_l
HSbrain_727_l
HSbrain_728_l
HSbrain_731_r
HSbrain_737_r
HSbrain_74_r
HSbrain_745_l
HSbrain_747_l
HSbrain_749_r
HSbrain_75_l
HSbrain_752_l
HSbrain_752_r
HSbrain_753_r
HSbrain_754_r
HSbrain_755_l
HSbrain_755_r
HSbrain_760_l
HSbrain_764_r
HSbrain_767_r
HSbrain_768_l
HSbrain_769_r
HSbrain_771_l
HSbrain_776_l
HSbrain_777_r
HSbrain_778_r
HSbrain_780_l
HSbrain_783_l
HSbrain_784_r
HSbrain_788_l
HSbrain_792_r
HSbrain_794_r
HSbrain_797_r
HSbrain_8_r
HSbrain_80_l
HSbrain_801_r
HSbrain_804_l
HSbrain_810_l
HSbrain_811_l
HSbrain_811_r
HSbrain_84_l
HSbrain_85_l
HSbrain_86_r
HSbrain_916_l
HSbrain_916_r
HSbrain_92_l
HSbrain_94_l
HSbrain_95_l
HSbrain_970_r
Hsd_101_l
Hsd_102_l
Hsd_105_l
Hsd_107_l
Hsd_108_r
Hsd_11_l
Hsd_111_r
Hsd_17_l
Hsd_17_r
Hsd_18_l
Hsd_18_r
Hsd_19_l
Hsd_19_r
Hsd_20_r
Hsd_25_l
Hsd_26_l
Hsd_27_r
Hsd_33_l
Hsd_37_l
Hsd_38_r
Hsd_42_l
Hsd_42_r
Hsd_46_l
Hsd_47_r
Hsd_49_r
Hsd_50_l
Hsd_6_l
Hsd_60_r
Hsd_62_l
Hsd_62_l
Hsd_63_l
Hsd_63_r
Hsd_65_l
Hsd_65_r
Hsd_67_r
Hsd_7_r
Hsd_70_l
Hsd_74_r
Hsd_76_r
Hsd_77_r
Hsd_79_l
Hsd_81_r
Hsd_82_r
Hsd_84_r
Hsd_88_r
Hsd_96_r
Hsd_from_Mmd_101_r
Hsd_from_Mmd_117_r
Hsd_from_Mmd_120_l
Hsd_from_Mmd_120_r
Hsd_from_Mmd_123_l
Hsd_from_Mmd_123_r
Hsd_from_Mmd_128_l
Hsd_from_Mmd_155_l
Hsd_from_Mmd_161_l
Hsd_from_Mmd_166_l
Hsd_from_Mmd_166_r
Hsd_from_Mmd_176_l
Hsd_from_Mmd_180_r
Hsd_from_Mmd_183_r
Hsd_from_Mmd_187_r
Hsd_from_Mmd_19_l
Hsd_from_Mmd_190_r
Hsd_from_Mmd_197_l
Hsd_from_Mmd_204_l
Hsd_from_Mmd_208_r
Hsd_from_Mmd_212_r
Hsd_from_Mmd_215_r
Hsd_from_Mmd_217_r
Hsd_from_Mmd_219_r
Hsd_from_Mmd_223_r
Hsd_from_Mmd_23_l
Hsd_from_Mmd_232_r
Hsd_from_Mmd_235_r
Hsd_from_Mmd_237_l
Hsd_from_Mmd_249_r
Hsd_from_Mmd_253_l
Hsd_from_Mmd_259_r
Hsd_from_Mmd_261_l
Hsd_from_Mmd_274_r
Hsd_from_Mmd_280_l
Hsd_from_Mmd_284_l
Hsd_from_Mmd_291_l
Hsd_from_Mmd_291_r
Hsd_from_Mmd_296_l
Hsd_from_Mmd_298_l
Hsd_from_Mmd_298_r
Hsd_from_Mmd_302_r
Hsd_from_Mmd_306_l
Hsd_from_Mmd_311_l
Hsd_from_Mmd_311_r
Hsd_from_Mmd_314_r
Hsd_from_Mmd_316_r
Hsd_from_Mmd_318_l
Hsd_from_Mmd_324_l
Hsd_from_Mmd_325_l
Hsd_from_Mmd_325_r
Hsd_from_Mmd_326_l
Hsd_from_Mmd_328_l
Hsd_from_Mmd_328_r
Hsd_from_Mmd_330_l
Hsd_from_Mmd_333_l
Hsd_from_Mmd_333_r
Hsd_from_Mmd_334_r
Hsd_from_Mmd_34_r
Hsd_from_Mmd_344_l
Hsd_from_Mmd_344_r
Hsd_from_Mmd_346_l
Hsd_from_Mmd_35_l
Hsd_from_Mmd_363_l
Hsd_from_Mmd_367_l
Hsd_from_Mmd_370_r
Hsd_from_Mmd_371_r
Hsd_from_Mmd_372_l
Hsd_from_Mmd_378_l
Hsd_from_Mmd_387_l
Hsd_from_Mmd_388_l
Hsd_from_Mmd_391_l
Hsd_from_Mmd_391_r
Hsd_from_Mmd_399_r
Hsd_from_Mmd_410_l
Hsd_from_Mmd_410_r
Hsd_from_Mmd_412_r
Hsd_from_Mmd_422_l
Hsd_from_Mmd_431_l
Hsd_from_Mmd_436_l
Hsd_from_Mmd_436_r
Hsd_from_Mmd_439_l
Hsd_from_Mmd_44_r
Hsd_from_Mmd_452_l
Hsd_from_Mmd_455_l
Hsd_from_Mmd_473_l
Hsd_from_Mmd_478_l
Hsd_from_Mmd_480_l
Hsd_from_Mmd_480_r
Hsd_from_Mmd_485_r
Hsd_from_Mmd_487_r
Hsd_from_Mmd_489_r
Hsd_from_Mmd_49_l
Hsd_from_Mmd_499_r
Hsd_from_Mmd_500_r
Hsd_from_Mmd_501_r
Hsd_from_Mmd_518_r
Hsd_from_Mmd_519_l
Hsd_from_Mmd_519_r
Hsd_from_Mmd_52_r
Hsd_from_Mmd_523_l
Hsd_from_Mmd_524_l
Hsd_from_Mmd_524_r
Hsd_from_Mmd_527_l
Hsd_from_Mmd_527_r
Hsd_from_Mmd_533_l
Hsd_from_Mmd_535_r
Hsd_from_Mmd_68_l
Hsd_from_Mmd_70_l
Hsd_from_Mmd_78_r
Hsd_from_Mmd_81_l
Hsd_from_Mmd_92_l
Hsd_from_Mmd_92_r
Hsd_from_Mmd_93_l
Hsd_from_Mmd_93_r
Hsd_from_Mmd_94_l
Hsd_from_Mmd_97_l

TABLE 3a
Small RNAs that are differentially expressed between control and
adenocarcinoma

| | carcinoma vs |
|---|---|
| Systematic Name | control adj p¹ |
| hsa-mir-573_l | 0.008895418 |
| HSbrain_801_r | 0.016433541 |
| HSbrain_377_l | 0.016433541 |
| HSbrain_426_r | 0.016433541 |
| HSbrain_148_r | 0.016433541 |
| hsa-mir-92b_r | 0.019274533 |
| hsa-mir-92-1_r | 0.019274533 |
| HS_from_PT_146_r | 0.019274533 |
| hsa-mir-92-2_r | 0.022921248 |
| HS_from_MQbrain_207_l | 0.022921248 |
| HS_from_PT_114_r | 0.027924358 |
| HS_from_PT_114_l | 0.027924358 |
| HS_from_MQbrain_100_l | 0.030988376 |
| hsa-mir-145_l | 0.031989408 |
| HS_from_MQbrain_350_l | 0.031989408 |
| hsa-mir-302d_l | 0.031989408 |
| HS_from_MQbrain_1628_r | 0.031989408 |
| HS_from_MQbrain_349_l | 0.031989408 |
| hsa-mir-325_l | 0.0342406 |
| HS_from_MQbrain_20_r | 0.0342406 |
| HSbrain_458_r | 0.035303066 |
| Hsd_102_l | 0.035303066 |
| HSbrain_377_r | 0.035303066 |
| HS_from_MQbrain_2700_r | 0.045228199 |
| HS_from_MQbrain_1687_l | 0.045228199 |
| HS_from_MQbrain_1311_r | 0.067337504 |
| Hsd_from_Mmd_519_l | 0.067337504 |
| HS_from_MQbrain_343_l | 0.07566513 |
| HS_from_MQbrain_1169_l | 0.084904114 |
| HS_from_MQbrain_965_l | 0.086503988 |
| HS_from_MM_178_r | 0.086503988 |
| hsa-mir-98_l | 0.086503988 |
| HS_from_MQbrain_549_l | 0.086503988 |
| hsa-mir-616_r | 0.090694253 |
| HSbrain_731_r | 0.090694253 |
| Hsd_from_Mmd_328_r | 0.090694253 |
| hsa-mir-768_r | 0.090694253 |
| HSbrain_709_l | 0.090694253 |
| HSbrain_398_r | 0.090694253 |
| HS_from_PT_340_l | 0.090694253 |
| HS_from_MQbrain_1884_r | 0.090694253 |
| hsa-let-7a-3_l | 0.090694253 |
| HSbrain_106_r | 0.099652833 |
| HS_from_MQbrain_2380_r | 0.099652833 |
| HSbrain_494_l | 0.104922786 |
| HSbrain_177_r | 0.104922786 |
| HSbrain_321_r | 0.104922786 |
| HS_from_MM_289_l | 0.104922786 |
| HS_from_MQbrain_191_l | 0.110995143 |
| HS_291_r | 0.110995143 |
| HS_from_MQbrain_128_r | 0.110995143 |
| hsa-let-7g_l | 0.110995143 |
| hsa-mir-633_l | 0.120015634 |
| HSbrain_500_r | 0.120015634 |
| HS_156_l | 0.120015634 |
| HS_from_MM_118_r | 0.120843678 |
| Hsd_from_Mmd_485_r | 0.120843678 |
| hsa-mir-655_l | 0.120843678 |
| HSbrain_617_l | 0.120843678 |
| HS_71_l | 0.120843678 |
| hsa-mir-20b_l | 0.120843678 |
| HS_from_MQbrain_71_r | 0.120843678 |
| hsa-mir-199a-2_r | 0.120843678 |
| hsa-mir-500_l | 0.120843678 |
| HSbrain_411_r | 0.120843678 |
| HS_from_MQbrain_930_r | 0.120843678 |
| hsa-mir-9-3_r | 0.120843678 |
| HS_from_MQbrain_769_r | 0.120843678 |
| hsa-mir-372_r | 0.120843678 |
| hsa-let-7a-2_l | 0.120843678 |
| hsa-let-7f-1_l | 0.120843678 |
| HSbrain_1437_r | 0.126655157 |
| hsa-mir-9-2_r | 0.126655157 |
| hsa-mir-181c_l | 0.126655157 |
| HS_from_MQbrain_1696_l | 0.126655157 |
| HS_from_MQbrain_572_l | 0.126655157 |
| hsa-let-7a-1_l | 0.126655157 |
| HS_from_MQbrain_1823_r | 0.149145996 |
| HS_from_MQbrain_1485_r | 0.149145996 |
| HS_from_MQbrain_1100_l | 0.149145996 |
| Hsd_18_l | 0.149145996 |
| hsa-mir-9-1_r | 0.149145996 |
| hsa-mir-29a_r | 0.149145996 |
| Hsd_111_r | 0.149145996 |
| hsa-mir-492_r | 0.159103307 |
| HSbrain_299_r | 0.159103307 |
| HSbrain_598_l | 0.159103307 |
| Hsd_26_l | 0.159103307 |
| HSbrain_261_l | 0.159103307 |
| hsa-mir-570_l | 0.166408872 |
| hsa-mir-363_l | 0.166408872 |
| hsa-mir-126_r | 0.166408872 |
| hsa-mir-192_r | 0.166408872 |
| HS_from_MQbrain_1459_l | 0.166408872 |
| HS_from_MQbrain_1624_l | 0.166408872 |
| HSbrain_458_l | 0.166408872 |
| HS_from_MQbrain_131_r | 0.179914563 |
| HSbrain_629_l | 0.179914563 |
| HS_304_r | 0.179914563 |
| hsa-let-7d_l | 0.179914563 |
| HS_141_l | 0.19263196 |
| HS_from_MQbrain_55_l | 0.19263196 |
| HSbrain_230_l | 0.19263196 |
| HSbrain_782_l | 0.19263196 |
| HS_from_MQbrain_1513_r | 0.19263196 |

¹Wilcoxon: cut off adjusted p value < 0.2

TABLE 3b
Up- or downregulated small RNAs in adenocarcinomas compared to
control

| | carcinoma | carcinomas | controls | |
|---|---|---|---|---|
| | vs control | (avg | (avg | carcinomas/controls |
| Systematic Name | (adj p)¹ | expression) | expression) | (Ratio expression)² |
| HS_71_l | 0.3 | −0.709677388 | 0.032756041 | −0.742433429 |
| HSbrain_458_r | 0.42 | −0.266452891 | 0.406777661 | −0.673230551 |
| hsa-mir-372_r | 0.12 | 0.060069121 | 0.629631623 | −0.569562502 |
| HS_from_MM_118_r | 0.69 | −0.237583049 | 0.179327953 | −0.416911001 |
| hsa-mir-192_r | 0.17 | −0.201240874 | 0.210651366 | −0.411892241 |
| Hsd_from_Mmd_485_r | 0.66 | −0.252579592 | 0.125681597 | −0.378261188 |
| HS_from_MQbrain_1624_l | 0.15 | −0.246477896 | 0.058106578 | −0.304584474 |
| HSbrain_629_l | 0.09 | −0.491364645 | −0.198765886 | −0.292598759 |
| hsa-mir-570_l | 0.17 | −0.130852458 | 0.019615717 | −0.150468175 |
| hsa-mir-492_r | 0.16 | −0.011846275 | 0.099781053 | −0.111627327 |
| HSbrain_731_r | 0.35 | 0.090460773 | −0.011239856 | 0.101700629 |
| HS_from_MQbrain_1687_l | 0.12 | −0.015384304 | −0.120029361 | 0.104645056 |
| HS_304_r | 0.18 | 0.725994298 | 0.518291894 | 0.207702404 |
| Hsd_18_l | 0.16 | 0.185427251 | −0.033505144 | 0.218932395 |
| HSbrain_177_r | 0.1 | −0.187948884 | −0.433561411 | 0.245612527 |
| Hsd_111_r | 0.15 | −0.186817378 | −0.43572008 | 0.248902702 |
| HS_from_MQbrain_131_r | 0.31 | 0.122733107 | −0.145810795 | 0.268543902 |
| HS_from_MQbrain_55_l | 0.09 | −0.014185779 | −0.291616347 | 0.277430568 |
| HS_from_MQbrain_207_l | 0.36 | 0.435514985 | 0.145542632 | 0.289972354 |
| HS_from_MQbrain_930_r | 0.12 | 0.389172004 | 0.087275842 | 0.301896162 |
| hsa-mir-500_l | 0.12 | −0.277848327 | −0.593537873 | 0.315689546 |
| HS_from_MQbrain_1884_r | 0.52 | 0.356353217 | 0.037880875 | 0.318472342 |
| HS_from_MQbrain_1513_r | 0.19 | −0.290006166 | −0.609064857 | 0.319058691 |
| HS_from_MQbrain_1459_l | 0.17 | 0.275771992 | −0.068316304 | 0.344088296 |
| HS_from_MQbrain_191_l | 0.68 | 0.374600902 | 0.027587412 | 0.34701349 |
| HS_from_MQbrain_2380_r | 0.09 | 0.419048482 | 0.049240856 | 0.369807627 |
| HS_from_MQbrain_769_r | 0.12 | −0.14336964 | −0.523899384 | 0.380529744 |
| Hsd_102_l | 0.04 | 0.443532 | 0.055860645 | 0.387671354 |
| HS_from_MQbrain_128_r | 0.11 | 0.617596666 | 0.225747368 | 0.391849298 |
| HS_141_l | 0.12 | 0.8422647 | 0.443686813 | 0.398577887 |
| HSbrain_709_l | 0.12 | 0.733394659 | 0.327477824 | 0.405916836 |
| HSbrain_321_r | 0.42 | 0.600940185 | 0.190823509 | 0.410116675 |
| HSbrain_617_l | 0.02 | 0.966810264 | 0.555939869 | 0.410870395 |
| HS_from_MQbrain_100_l | 0.42 | 0.278755917 | −0.134292565 | 0.413048482 |
| HSbrain_299_r | 0.16 | 0.470656977 | 0.050970084 | 0.419686894 |
| HSbrain_782_l | 0.19 | 0.394023714 | −0.030111689 | 0.424135402 |
| HSbrain_230_l | 0.19 | 0.420548412 | −0.007405663 | 0.427954074 |
| HS_from_MQbrain_343_l | 0.08 | 0.191928075 | −0.240864595 | 0.432792669 |
| hsa-mir-616_r | 0.65 | −0.186021179 | −0.619960119 | 0.43393894 |
| HSbrain_598_l | 0.16 | 0.432451861 | −0.001594407 | 0.434046268 |
| hsa-let-7d_l | 0.18 | −0.214090094 | −0.663813813 | 0.449723719 |
| HS_from_MQbrain_20_r | 0.25 | −0.154082272 | −0.608413911 | 0.454331639 |
| HS_from_MQbrain_350_l | 0.09 | 0.146762687 | −0.311281719 | 0.458044406 |
| HSbrain_494_l | 0.1 | 0.06720096 | −0.396374725 | 0.463575685 |
| HSbrain_801_r | 0.15 | 0.33891208 | −0.13043269 | 0.46934477 |
| hsa-mir-655_l | 0.13 | −0.049016209 | −0.523332673 | 0.474316464 |
| HS_from_PT_146_r | 0.41 | 0.344948777 | −0.130022489 | 0.474971266 |
| Hsd_26_l | 0.58 | 0.185069914 | −0.292706346 | 0.47777626 |
| HS_from_MQbrain_349_l | 0.09 | 0.181998814 | −0.301075838 | 0.483074652 |
| HSbrain_148_r | 0.3 | 0.440551449 | −0.043142521 | 0.48369397 |
| HSbrain_106_r | 0.33 | 0.575843279 | 0.080837435 | 0.495005844 |
| hsa-mir-633_l | 0.03 | 0.215123255 | −0.284566041 | 0.499689296 |
| HS_from_MM_289_l | 0.1 | 0.092159319 | −0.424119594 | 0.516278912 |
| HS_from_MQbrain_71_r | 0.24 | 0.86119198 | 0.341225696 | 0.519966285 |
| HSbrain_261_l | 0.16 | 0.200748997 | −0.325726286 | 0.526475284 |
| Hsd_from_Mmd_328_r | 0.09 | 0.336779316 | −0.20245187 | 0.539231186 |
| Hsd_from_Mmd_519_l | 0.42 | 0.156016612 | −0.390158334 | 0.546174946 |
| HS_from_MM_178_r | 0.03 | 0.215653589 | −0.344575388 | 0.560228977 |
| HSbrain_458_l | 0.17 | −0.623680905 | −1.206064715 | 0.58238381 |
| HS_from_MQbrain_1485_r | 0.28 | 0.341195456 | −0.243332697 | 0.584528153 |
| HS_from_MQbrain_1628_r | 0.79 | 0.139811659 | −0.450916985 | 0.590728644 |
| hsa-mir-302d_l | 0.37 | −0.417071628 | −1.008783793 | 0.591712165 |
| HS_156_l | 0.12 | −0.025280933 | −0.617537087 | 0.592256154 |
| HS_from_MQbrain_1823_r | 0.03 | −0.296865659 | −0.892007201 | 0.595141542 |
| hsa-mir-325_l | 0.69 | 0.363886014 | −0.231356523 | 0.595242537 |
| HS_from_MQbrain_1696_l | 0.13 | −0.405403011 | −1.024758234 | 0.619355223 |
| HS_from_MQbrain_549_l | 0.37 | 0.266837912 | −0.360518665 | 0.627356576 |
| HS_from_MQbrain_1311_r | 0.64 | 0.085649422 | −0.542358185 | 0.628007606 |
| HS_from_PT_340_l | 0.09 | 0.304986748 | −0.325416792 | 0.630403539 |
| HS_from_MQbrain_1100_l | 0.3 | 0.502916758 | −0.128399246 | 0.631316004 |
| HSbrain_426_r | 0.37 | 0.499080863 | −0.16430699 | 0.663387852 |
| HS_from_MQbrain_965_l | 0.75 | 0.559300427 | −0.137794367 | 0.697094794 |
| hsa-mir-573_l | 0.03 | −0.059472954 | −0.78119509 | 0.721722137 |
| HS_from_MQbrain_1169_l | 0.08 | 0.560023874 | −0.172737263 | 0.732761137 |
| hsa-mir-126_r | 0.42 | −0.201075637 | −0.937409046 | 0.736333409 |
| hsa-mir-98_l | 0.86 | −0.358721365 | −1.199711347 | 0.840989982 |
| hsa-let-7a-2_l | 0.54 | −0.252165432 | −1.106318992 | 0.85415356 |
| HSbrain_398_r | 0.74 | 0.878616993 | 0.004337368 | 0.874279624 |
| hsa-let-7a-1_l | 0.12 | −0.130600434 | −1.038969263 | 0.908368829 |
| hsa-let-7a-3_l | 0.73 | −0.139853217 | −1.051229242 | 0.911376026 |
| HSbrain_500_r | 0.77 | 0.944568594 | 0.020888827 | 0.923679767 |
| hsa-mir-199a-2_r | 0.12 | −0.169461658 | −1.102399679 | 0.932938022 |
| hsa-let-7g_l | 0.34 | −0.324092373 | −1.332989933 | 1.008897559 |
| HS_291_r | 0.3 | 0.833287938 | −0.193284833 | 1.026572771 |
| hsa-mir-768_r | 0.73 | 0.861619701 | −0.182102011 | 1.043721712 |
| HSbrain_1437_r | 0.79 | 0.870808091 | −0.201624857 | 1.072432947 |
| hsa-mir-181c_l | 0.55 | −0.036071696 | −1.122096366 | 1.086024669 |
| hsa-let-7f-1_l | 0.3 | −0.079122123 | −1.192010342 | 1.112888218 |
| HS_from_MQbrain_572_l | 0.13 | 0.0201842 | −1.13040159 | 1.15058579 |
| HS_from_MQbrain_2700_r | 0.58 | 0.98864825 | −0.187941077 | 1.176589327 |
| HSbrain_411_r | 0.02 | 0.267080657 | −0.993945285 | 1.261025942 |
| hsa-mir-363_l | 0.17 | 1.940171184 | 0.587265163 | 1.352906021 |
| hsa-mir-92b_r | 0.02 | 0.784118073 | −0.596107394 | 1.380225467 |
| hsa-mir-20b_l | 0.12 | −0.302677154 | −1.731722022 | 1.429044868 |
| hsa-mir-29a_r | 0.05 | −0.289379384 | −1.809571559 | 1.520192175 |
| hsa-mir-9-2_r | 0.13 | 2.216369234 | 0.500287664 | 1.71608157 |
| hsa-mir-9-1_r | 0.15 | 2.217636141 | 0.490685702 | 1.726950439 |
| HSbrain_377_r | 0.02 | 1.13757316 | −0.614439492 | 1.752012652 |
| hsa-mir-9-3_r | 0.12 | 2.225977245 | 0.459667748 | 1.766309498 |
| hsa-mir-145_l | 0.03 | 0.930167523 | −0.909054029 | 1.839221552 |
| HSbrain_377_l | 0.28 | 0.800203766 | −1.135562989 | 1.935766755 |
| hsa-mir-92-2_r | 0.21 | 1.186285863 | −0.928692902 | 2.114978765 |
| hsa-mir-92-1_r | 0.73 | 1.223717878 | −0.942146802 | 2.16586468 |
| HS_from_PT_114_l | 0.3 | 0.966988882 | −1.364997051 | 2.331985933 |
| HS_from_PT_114_r | 0.57 | 1.256897347 | −1.276764924 | 2.533662271 |

¹Wilcoxon: cut off adjusted p value < 0.2
²Upregulated small RNAs have a ratio for expression >1 (below line in table)
Downregulated small RNAs have a ratio for expression <1 (above line in table)

TABLE 3c
Small RNAs that are upregulated in colorectal adenocarcinomas
compared to control samples

| | | |
|---|---|---|
| hsa-let-7g_l | HSbrain_411_r | hsa-mir-9-3_r |
| HS_291_r | hsa-mir-363_l | hsa-mir-145_l |
| hsa-mir-768_r | hsa-mir-92b_r | HSbrain_377_l |
| HSbrain_1437_r | hsa-mir-20b_l | hsa-mir-92-2_r |
| hsa-mir-181c_l | hsa-mir-29a_r | hsa-mir-92-1_r |
| hsa-let-7f-1_l | hsa-mir-9-2_r | HS_from_PT_114_l |
| HS_from_MQbrain_572_l | hsa-mir-9-1_r | HS_from_PT_114_r |
| HS_from_MQbrain_2700_r | HSbrain_377_r | |

TABLE 3d
Small RNAs that are downregulated in colorectal
adenocarcinomas compared to control samples

| | | |
|---|---|---|
| HS_141_l | HS_from_PT_340_l | HSbrain_731_r |
| HS_156_l | hsa-let-7a-1_l | HSbrain_782_l |
| HS_304_r | hsa-let-7a-2_l | HSbrain_801_r |
| HS_71_l | hsa-let-7a-3_l | Hsd_102_l |
| HS_from_MM_118_r | hsa-let-7d_l | Hsd_111_r |
| HS_from_MM_178_r | hsa-mir-126_r | Hsd_18_l |
| HS_from_MM_289_l | hsa-mir-192_r | Hsd_26_l |
| HS_from_MQbrain_100_l | hsa-mir-199a-2_r | Hsd_from_Mmd_328_r |
| HS_from_MQbrain_1100_l | hsa-mir-302d_l | Hsd_from_Mmd_485_r |
| HS_from_MQbrain_1169_l | hsa-mir-325_l | Hsd_from_Mmd_519_l |
| HS_from_MQbrain_128_r | hsa-mir-372_r | |
| HS_from_MQbrain_131_r | hsa-mir-492_r | |
| HS_from_MQbrain_1311_r | hsa-mir-500_l | |
| HS_from_MQbrain_1459_l | hsa-mir-570_l | |
| HS_from_MQbrain_1485_r | hsa-mir-573_l | |
| HS_from_MQbrain_1513_r | hsa-mir-616_r | |
| HS_from_MQbrain_1624_l | hsa-mir-633_l | |
| HS_from_MQbrain_1628_r | hsa-mir-655_l | |
| HS_from_MQbrain_1687_l | hsa-mir-98_l | |
| HS_from_MQbrain_1696_l | HSbrain_106_r | |
| HS_from_MQbrain_1823_r | HSbrain_148_r | |
| HS_from_MQbrain_1884_r | HSbrain_177_r | |
| HS_from_MQbrain_191_l | HSbrain_230_l | |
| HS_from_MQbrain_20_r | HSbrain_261_l | |
| HS_from_MQbrain_207_l | HSbrain_299_r | |
| HS_from_MQbrain_2380_r | HSbrain_321_r | |
| HS_from_MQbrain_343_l | HSbrain_398_r | |
| HS_from_MQbrain_349_l | HSbrain_426_r | |
| HS_from_MQbrain_350_l | HSbrain_458_l | |
| HS_from_MQbrain_549_l | HSbrain_458_r | |
| HS_from_MQbrain_55_l | HSbrain_494_l | |
| HS_from_MQbrain_71_r | HSbrain_500_r | |
| HS_from_MQbrain_769_r | HSbrain_598_l | |
| HS_from_MQbrain_930_r | HSbrain_617_l | |
| HS_from_MQbrain_965_l | HSbrain_629_l | |
| HS_from_PT_146_r | HSbrain_709_l | |

TABLE 4a
Small RNAs that are differentially expressed between adenoma and
adenocarcinoma

| | adenocarcinoma |
|---|---|
| | vs adenoma |
| Systematic Name | (adj p)¹ |
| HS_from_MQbrain_1426_r | 0.00750624 |
| HS_from_MQbrain_638_l | 0.00750624 |
| HS_from_MQbrain_1391_l | 0.012664553 |
| HS_from_MQbrain_420_l | 0.012664553 |
| HS_from_PT_36_l | 0.012664553 |
| HSbrain_390_l | 0.012664553 |
| HS_from_MM_242_r | 0.014169909 |
| HS_136_r | 0.014224152 |
| HS_8_r | 0.014224152 |
| HS_from_PT_201_l | 0.014224152 |
| HSbrain_545_r | 0.014224152 |
| HS_176_l | 0.014416465 |
| HS_71_r | 0.014416465 |
| HS_from_MM_169_r | 0.014416465 |
| HS_from_MM_92_r | 0.015226536 |
| HSbrain_293_l | 0.015226536 |
| HS_from_MQbrain_1334_r | 0.015599346 |
| HS_from_MQbrain_590_l | 0.015599346 |
| HS_from_MQbrain_63_r | 0.015599346 |
| HSbrain_334_r | 0.015599346 |
| HS_299_l | 0.016786613 |
| HS_276_l | 0.016904198 |
| HS_from_MQbrain_1137_l | 0.016904198 |
| HS_from_MQbrain_377_l | 0.016904198 |
| HS_from_MQbrain_941_r | 0.016904198 |
| HS_from_MQbrain_1082_l | 0.018319912 |
| HS_from_MQbrain_1792_r | 0.018494113 |
| HS_from_MQbrain_458_l | 0.018494113 |
| hsa-mir-24-2_l | 0.018494113 |
| HS_157_r | 0.020545593 |
| HS_from_MM_295_l | 0.020545593 |
| HS_from_MQbrain_1297_l | 0.020545593 |
| HSbrain_217_r | 0.020545593 |
| HS_from_MQbrain_1072_l | 0.021132076 |
| HS_from_MQbrain_1350_l | 0.022396706 |
| HS_from_PT_226_l | 0.022396706 |
| HS_from_MQbrain_1500_l | 0.023919533 |
| hsa-mir-346_l | 0.023919533 |
| hsa-mir-767_r | 0.023919533 |
| HSbrain_306_r | 0.023919533 |
| hsa-mir-324_r | 0.024694474 |
| HS_124_r | 0.024910624 |
| HS_51_r | 0.024910624 |
| HS_from_MQbrain_1363_l | 0.02517728 |
| HS_from_MQbrain_1620_r | 0.02517728 |
| HSbrain_811_l | 0.026045397 |
| HS_from_MQbrain_825_r | 0.026388496 |
| Hsd_from_Mmd_157_r | 0.026388496 |
| HS_from_MQbrain_133_r | 0.026776445 |
| HSbrain_583_l | 0.026776445 |
| HSbrain_796_r | 0.027740873 |
| HS_from_MQbrain_441_l | 0.028202169 |
| hsa-mir-653_l | 0.028202169 |
| HS_from_MQbrain_1485_l | 0.029237065 |
| HS_from_MQbrain_1042_r | 0.030313454 |
| HSbrain_680_r | 0.031432755 |
| HS_from_MQbrain_828_l | 0.032034428 |
| HSbrain_399_l | 0.032034428 |
| HS_from_PT_187_r | 0.032679147 |
| hsa-mir-453_l | 0.032679147 |
| hsa-mir-548b_l | 0.033913448 |
| HS_from_MQbrain_1204_l | 0.034662803 |
| HS_from_MQbrain_1469_l | 0.034662803 |
| hsa-mir-331_l | 0.034662803 |
| hsa-mir-523_r | 0.034662803 |
| HSbrain_384_l | 0.034662803 |
| HSbrain_570_r | 0.034662803 |
| HSbrain_687_r | 0.034662803 |
| HSbrain_7_r | 0.034662803 |
| Hsd_85_r | 0.034662803 |
| HS_from_MQbrain_1099_r | 0.035037528 |
| hsa-mir-412_l | 0.035037528 |
| Hsd_70_r | 0.035037528 |
| HS_46_l | 0.035941334 |
| HSbrain_1425_l | 0.035941334 |
| HS_from_MQbrain_1781_l | 0.036886609 |
| HS_from_PT_250_l | 0.036886609 |
| HS_from_MM_174_l | 0.037874034 |
| HS_from_MQbrain_1750_r | 0.037874034 |
| HS_from_MM_251_r | 0.039250091 |
| HS_from_MM_84_r | 0.039250091 |
| HS_from_MQbrain_43_r | 0.039250091 |
| HS_from_MQbrain_984_l | 0.039250091 |
| hsa-mir-375_r | 0.039250091 |
| hsa-mir-377_l | 0.039250091 |
| HSbrain_272_l | 0.039250091 |
| HSbrain_276_l | 0.039250091 |
| HSbrain_351_r | 0.039250091 |
| Hsd_18_r | 0.039250091 |
| HSbrain_1325_r | 0.040852758 |
| HS_from_MQbrain_365_r | 0.042516991 |
| HS_from_MM_118_r | 0.044599949 |
| HS_from_MQbrain_603_r | 0.044599949 |
| HS_from_MQbrain_686_r | 0.044599949 |
| HS_from_MQbrain_854_r | 0.044599949 |
| HSbrain_669_r | 0.044599949 |
| HS_from_PT_287_r | 0.045945376 |
| Hsd_from_Mmd_452_l | 0.045945376 |
| hsa-mir-136_l | 0.047341086 |
| hsa-mir-383_r | 0.047341086 |
| HS_from_MQbrain_1985_l | 0.047394461 |
| HS_from_MQbrain_2669_l | 0.047394461 |
| HSbrain_476_r | 0.047394461 |
| HSbrain_77_l | 0.047394461 |
| Hsd_108_l | 0.047394461 |
| HS_281_r | 0.04754573 |
| HS_from_MQbrain_1253_r | 0.04754573 |
| hsa-mir-661_r | 0.04754573 |
| HSbrain_304_r | 0.04754573 |
| HSbrain_438_r | 0.04754573 |
| HS_213_l | 0.048205391 |
| HS_79_l | 0.048205391 |
| HS_from_MQbrain_542_r | 0.048205391 |
| HSbrain_177_l | 0.048205391 |
| HS_from_MQbrain_1212_l | 0.049767746 |
| HSbrain_122_l | 0.049767746 |
| HS_212_l | 0.050135128 |
| HS_from_MQbrain_1565_l | 0.050135128 |
| HS_from_MQbrain_163_l | 0.050135128 |
| hsa-let-7c_r | 0.050135128 |
| hsa-mir-591_r | 0.050135128 |
| HSbrain_187_r | 0.050135128 |
| HSbrain_226_r | 0.050135128 |
| HSbrain_437_r | 0.050135128 |
| HSbrain_780_r | 0.050135128 |
| Hsd_from_Mmd_166_l | 0.050135128 |
| Hsd_from_Mmd_485_r | 0.050135128 |
| HS_from_MQbrain_660_l | 0.051819249 |
| HS_from_PT_101_r | 0.051819249 |
| hsa-mir-148a_l | 0.052756328 |
| HSbrain_252_l | 0.052756328 |
| HSbrain_673_r | 0.052756328 |
| HSbrain_684_r | 0.052756328 |
| HS_17_l | 0.053747925 |
| HS_from_MQbrain_1444_l | 0.053747925 |
| HSbrain_494_l | 0.053747925 |
| HSbrain_55_l | 0.053747925 |
| HS_from_MM_218_l | 0.055185029 |
| HS_from_MQbrain_1263_r | 0.055185029 |
| HSbrain_538_r | 0.055185029 |
| HSbrain_803_l | 0.057478906 |
| HS_189_r | 0.058218978 |
| HS_from_MQbrain_71_l | 0.058218978 |
| hsa-mir-411_r | 0.058218978 |
| hsa-mir-497_r | 0.058218978 |
| Hsd_from_Mmd_371_r | 0.058218978 |
| HS_from_MQbrain_1340_l | 0.058254254 |
| HS_from_PT_308_l | 0.058254254 |
| hsa-mir-638_r | 0.058254254 |
| HSbrain_374_r | 0.058254254 |
| HSbrain_420_r | 0.058254254 |
| HSbrain_583_r | 0.058254254 |
| Hsd_from_Mmd_49_l | 0.058254254 |
| HS_262_l | 0.059897432 |
| HS_from_MQbrain_346_l | 0.059897432 |
| HSbrain_791_l | 0.059897432 |
| HSbrain_25_r | 0.062381992 |
| hsa-mir-301_l | 0.06455063 |
| Hsd_from_Mmd_413_r | 0.06455063 |
| HS_from_MQbrain_209_r | 0.0659723 |
| HS_from_MQbrain_579_l | 0.0659723 |
| hsa-mir-659_l | 0.0659723 |
| HSbrain_1076_r | 0.0659723 |
| HS_from_MQbrain_65_l | 0.068270026 |
| HS_from_MQbrain_932_l | 0.068270026 |
| HS_261_r | 0.068590471 |
| HS_from_MQbrain_108_r | 0.068590471 |
| HS_from_MQbrain_1134_r | 0.068590471 |
| HS_from_MQbrain_120_l | 0.068590471 |
| hsa-mir-133a-1_r | 0.068590471 |
| hsa-mir-155_r | 0.068590471 |
| hsa-mir-604_l | 0.068590471 |
| HS_from_MQbrain_664_r | 0.070590475 |
| Hsd_88_r | 0.070590475 |
| Hsd_from_Mmd_391_l | 0.070590475 |
| HS_from_MQbrain_1832_l | 0.071451516 |
| HS_from_MQbrain_58_l | 0.071451516 |
| HS_from_MQbrain_736_r | 0.071451516 |
| HS_from_MQbrain_93_r | 0.071451516 |
| HSbrain_468_r | 0.071451516 |
| Hsd_from_Mmd_237_l | 0.071451516 |
| HS_from_MM_1_l | 0.072275086 |
| HS_from_MM_39_l | 0.072275086 |
| HS_from_MQbrain_1006_l | 0.072275086 |
| HS_from_MQbrain_1958_l | 0.072275086 |
| HS_from_MQbrain_504_r | 0.072275086 |
| HS_from_MQbrain_921_r | 0.072275086 |
| HS_from_PT_237_r | 0.072275086 |
| HS_from_PT_299_r | 0.072275086 |
| hsa-mir-124a-1_l | 0.072275086 |
| HSbrain_182_r | 0.072275086 |
| HSbrain_518_r | 0.072275086 |
| HSbrain_62_r | 0.072275086 |
| HSbrain_93_r | 0.072275086 |
| Hsd_107_l | 0.072275086 |
| Hsd_from_Mmd_222_l | 0.072275086 |
| HS_200_r | 0.072654718 |
| HS_from_MM_232_r | 0.072654718 |
| HS_from_MQbrain_1246_l | 0.072654718 |
| HS_from_MQbrain_1301_r | 0.072654718 |
| HS_from_MQbrain_301_l | 0.072654718 |
| HS_from_MQbrain_435_r | 0.072654718 |
| HS_from_MQbrain_869_l | 0.072654718 |
| HSbrain_1406_l | 0.072654718 |
| HS_from_MM_339_l | 0.074901054 |
| HS_from_MQbrain_74_r | 0.074901054 |
| HSbrain_419_l | 0.074901054 |
| HS_219_l | 0.075028175 |
| HS_307_r | 0.075028175 |
| HS_from_MM_103_l | 0.075028175 |
| HS_from_MM_32_r | 0.075028175 |
| HS_from_MQbrain_1001_r | 0.075028175 |
| HS_from_MQbrain_1740_r | 0.075028175 |
| HS_from_MQbrain_266_r | 0.075028175 |
| HS_from_MQbrain_470_l | 0.075028175 |
| HS_from_MQbrain_969_l | 0.075028175 |
| HS_from_PT_254_l | 0.075028175 |
| hsa-mir-1-1_l | 0.075028175 |
| hsa-mir-130b_l | 0.075028175 |
| hsa-mir-365-2_l | 0.075028175 |
| hsa-mir-98_r | 0.075028175 |
| HSbrain_1298_r | 0.075028175 |
| HSbrain_47_r | 0.075028175 |
| HSbrain_580_r | 0.075028175 |
| Hsd_from_Mmd_215_r | 0.075028175 |
| Hsd_from_Mmd_473_l | 0.075028175 |
| hsa-mir-190_r | 0.07641095 |
| hsa-mir-619_l | 0.07641095 |
| HSbrain_6_r | 0.07641095 |
| Hsd_from_Mmd_189_l | 0.07641095 |
| Hsd_from_Mmd_410_r | 0.07641095 |
| Hsd_from_Mmd_464_l | 0.07641095 |
| HS_118_l | 0.07715065 |
| HS_119_l | 0.07715065 |
| HS_292_l | 0.07715065 |
| HS_from_MM_18_r | 0.07715065 |
| HS_from_MM_275_r | 0.07715065 |
| HS_from_MM_342_r | 0.07715065 |
| HS_from_MQbrain_117_r | 0.07715065 |
| HS_from_MQbrain_154_r | 0.07715065 |
| HS_from_MQbrain_1896_r | 0.07715065 |
| HS_from_MQbrain_881_r | 0.07715065 |
| HS_from_MQbrain_972_r | 0.07715065 |
| hsa-mir-299_l | 0.07715065 |
| hsa-mir-433_r | 0.07715065 |
| HSbrain_204_r | 0.07715065 |
| HSbrain_231_l | 0.07715065 |
| HSbrain_250_r | 0.07715065 |
| HSbrain_431_r | 0.07715065 |
| HSbrain_703_r | 0.07715065 |
| Hsd_105_l | 0.07715065 |
| hsa-mir-362_r | 0.07965977 |
| HSbrain_319_l | 0.07965977 |
| HSbrain_442_r | 0.07965977 |
| HS_95_r | 0.080676612 |
| HS_from_MQbrain_355_l | 0.080676612 |
| HS_from_PT_196_l | 0.080676612 |
| HS_from_PT_308_l | 0.080676612 |
| HSbrain_236_l | 0.080676612 |
| HSbrain_361_l | 0.080676612 |
| HSbrain_473_l | 0.080676612 |
| HSbrain_792_r | 0.080676612 |
| HS_from_MQbrain_1260_r | 0.08237725 |
| HS_from_PT_27_r | 0.08237725 |
| HSbrain_1360_r | 0.08237725 |
| HSbrain_328_l | 0.08237725 |
| HSbrain_757_r | 0.08237725 |
| HSbrain_799_r | 0.08237725 |
| HS_67_r | 0.083833911 |
| HS_from_MQbrain_1087_l | 0.083833911 |
| HS_from_MQbrain_1374_r | 0.083833911 |
| HS_from_MQbrain_1569_l | 0.083833911 |
| HS_from_MQbrain_634_r | 0.083833911 |
| HSbrain_627_l | 0.083833911 |
| Hsd_42_l | 0.083833911 |
| HS_54_r | 0.084885584 |
| HS_from_MM_18_l | 0.084885584 |
| HS_from_MQbrain_1294_l | 0.084885584 |
| HS_from_MQbrain_1443_r | 0.084885584 |
| HS_from_MQbrain_1494_r | 0.084885584 |
| HS_from_MQbrain_1623_r | 0.084885584 |
| HS_from_MQbrain_1675_r | 0.084885584 |
| HS_from_MQbrain_597_r | 0.084885584 |
| HS_from_MQbrain_823_l | 0.084885584 |
| HS_from_MQbrain_824_l | 0.084885584 |
| HS_from_MQbrain_901_r | 0.084885584 |
| hsa-mir-146a_r | 0.084885584 |
| hsa-mir-412_r | 0.084885584 |
| hsa-mir-652_r | 0.084885584 |
| HSbrain_1423_l | 0.084885584 |
| HSbrain_389_l | 0.084885584 |
| HSbrain_95_l | 0.084885584 |
| Hsd_106_l | 0.084885584 |
| Hsd_from_Mmd_261_l | 0.084885584 |
| Hsd_from_Mmd_302_r | 0.084885584 |
| Hsd_from_Mmd_44_r | 0.084885584 |
| HS_208_l | 0.085697388 |
| HS_83_r | 0.085697388 |
| HS_from_MQbrain_330_r | 0.085697388 |
| HS_from_MQbrain_468_r | 0.085697388 |
| HS_from_MQbrain_534_l | 0.085697388 |
| HS_from_MQbrain_928_r | 0.085697388 |
| hsa-let-7a-1_r | 0.085697388 |
| hsa-mir-584_r | 0.085697388 |
| HSbrain_764_r | 0.085697388 |
| Hsd_101_l | 0.085697388 |
| HS_49_r | 0.088269125 |
| HS_from_MQbrain_1593_r | 0.088269125 |
| HS_from_PT_13_l | 0.088269125 |
| Hsd_from_Mmd_511_r | 0.088269125 |
| HS_from_MQbrain_1355_r | 0.089491072 |
| HS_from_MQbrain_1481_l | 0.089491072 |
| HS_from_MQbrain_597_l | 0.089491072 |
| hsa-mir-624_l | 0.089491072 |
| HSbrain_365_l | 0.089491072 |
| HSbrain_512_l | 0.089491072 |
| HSbrain_695_l | 0.089491072 |
| HSbrain_705_l | 0.089491072 |
| Hsd_27_r | 0.089491072 |
| HS_145_r | 0.091337111 |
| HS_151_r | 0.091337111 |
| HS_from_MM_219_l | 0.091337111 |
| HS_from_MM_72_r | 0.091337111 |
| HS_from_MQbrain_1771_r | 0.091337111 |
| HS_from_MQbrain_1877_l | 0.091337111 |
| HS_from_MQbrain_392_l | 0.091337111 |
| HS_from_MQbrain_631_l | 0.091337111 |
| HS_from_MQbrain_72_r | 0.091337111 |
| HS_from_MQbrain_723_l | 0.091337111 |
| HS_from_MQbrain_765_l | 0.091337111 |
| HS_from_MQbrain_81_l | 0.091337111 |
| HS_from_PT_282_l | 0.091337111 |
| HSbrain_359_l | 0.091337111 |
| HSbrain_371_r | 0.091337111 |
| HSbrain_472_l | 0.091337111 |
| HSbrain_594_l | 0.091337111 |
| HSbrain_717_r | 0.091337111 |
| Hsd_46_l | 0.091337111 |
| Hsd_from_Mmd_324_l | 0.091337111 |
| Hsd_from_Mmd_93_r | 0.091337111 |
| HS_14_r | 0.092780591 |
| HS_from_MQbrain_1264_l | 0.092780591 |
| HS_from_MQbrain_566_l | 0.092780591 |
| HS_from_MQbrain_634_l | 0.092780591 |
| HSbrain_114_r | 0.092780591 |
| HSbrain_773_r | 0.092780591 |
| Hsd_from_Mmd_225_l | 0.092780591 |
| Hsd_from_Mmd_422_l | 0.092780591 |
| Hsd_from_Mmd_491_r | 0.092780591 |
| HS_171_r | 0.094556199 |
| HS_from_MQbrain_1901_r | 0.094556199 |
| HSbrain_135_l | 0.094556199 |
| HSbrain_465_l | 0.094556199 |
| HSbrain_591_l | 0.094556199 |
| HSbrain_728_l | 0.094556199 |
| HSbrain_754_r | 0.094556199 |
| Hsd_from_Mmd_330_l | 0.094556199 |
| HS_74_r | 0.096132958 |
| HS_from_MM_121_l | 0.096132958 |
| HS_from_MQbrain_1717_r | 0.096132958 |
| HS_from_MQbrain_1793_r | 0.096132958 |
| hsa-mir-224_r | 0.096132958 |
| HSbrain_123_l | 0.096132958 |
| HSbrain_532_l | 0.096132958 |
| Hsd_30_l | 0.096132958 |
| Hsd_83_l | 0.096132958 |
| HS_from_MQbrain_1771_l | 0.098301345 |
| HS_from_MQbrain_181_l | 0.098301345 |
| HS_from_MQbrain_1893_r | 0.098301345 |
| HSbrain_316_l | 0.098301345 |
| HSbrain_452_l | 0.098301345 |
| HSbrain_52_r | 0.098301345 |
| HSbrain_553_r | 0.098301345 |
| HS_288_r | 0.099488281 |
| HS_86_r | 0.099488281 |
| HS_from_MM_150_r | 0.099488281 |
| HS_from_MQbrain_1031_r | 0.099488281 |
| HS_from_MQbrain_1649_r | 0.099488281 |
| HS_from_MQbrain_1840_l | 0.099488281 |
| hsa-mir-193a_l | 0.099488281 |
| hsa-mir-646_r | 0.099488281 |
| HSbrain_235_l | 0.099488281 |
| HSbrain_719_l | 0.099488281 |
| Hsd_from_Mmd_524_l | 0.099488281 |
| HS_from_MM_84_l | 0.101009439 |
| HS_from_MQbrain_1085_l | 0.101009439 |
| HS_from_MQbrain_1700_r | 0.101009439 |
| HS_from_MQbrain_1748_r | 0.101009439 |
| HS_from_MQbrain_1806_l | 0.101009439 |
| HS_from_MQbrain_1827_l | 0.101009439 |
| HS_from_PT_140_l | 0.101009439 |
| HSbrain_14_r | 0.101009439 |
| HSbrain_703_l | 0.101009439 |
| Hsd_from_Mmd_346_l | 0.101009439 |
| HS_287_r | 0.102601153 |
| HS_from_MQbrain_129_r | 0.102601153 |
| HS_from_MQbrain_1677_r | 0.102601153 |
| HS_from_MQbrain_221_r | 0.102601153 |
| HS_from_MQbrain_628_l | 0.102601153 |
| HS_from_MQbrain_716_l | 0.102601153 |
| HSbrain_507_l | 0.102601153 |
| HSbrain_661_l | 0.102601153 |
| Hsd_from_Mmd_117_r | 0.102601153 |
| Hsd_from_Mmd_138_l | 0.102601153 |
| HS_from_MQbrain_1116_r | 0.104515233 |
| HS_from_MQbrain_1374_l | 0.104515233 |
| HS_from_MQbrain_433_l | 0.104515233 |
| HS_from_MQbrain_62_l | 0.104515233 |
| HS_from_MQbrain_722_r | 0.104515233 |
| hsa-mir-633_r | 0.104515233 |
| HSbrain_266_r | 0.104515233 |
| Hsd_37_r | 0.104515233 |
| Hsd_from_Mmd_405_l | 0.104515233 |
| HS_99_r | 0.105993846 |
| HS_from_MQbrain_1127_r | 0.105993846 |
| HS_from_MQbrain_1537_r | 0.105993846 |
| HS_from_MQbrain_1545_r | 0.105993846 |
| HS_from_MQbrain_1783_r | 0.105993846 |
| HS_from_MQbrain_22_r | 0.105993846 |
| HS_from_MQbrain_534_r | 0.105993846 |
| HS_from_MQbrain_852_l | 0.105993846 |
| HS_from_MQbrain_900_r | 0.105993846 |
| hsa-let-7f-1_r | 0.105993846 |
| Hsd_from_Mmd_166_r | 0.105993846 |
| HS_from_MQbrain_1541_l | 0.107794024 |
| HS_from_MQbrain_574_l | 0.107794024 |
| HS_from_MQbrain_921_l | 0.107794024 |
| HS_from_MQbrain_951_l | 0.107794024 |
| HS_from_MQbrain_996_l | 0.107794024 |
| hsa-mir-625_r | 0.107794024 |
| HSbrain_106_r | 0.107794024 |
| HSbrain_184_r | 0.107794024 |
| HSbrain_696_r | 0.107794024 |
| Hsd_from_Mmd_255_l | 0.107794024 |
| HS_from_MM_86_l | 0.11066006 |
| HS_from_MQbrain_1339_r | 0.11066006 |
| HS_from_MQbrain_2087_r | 0.11066006 |
| HSbrain_677_l | 0.11066006 |
| Hsd_from_Mmd_266_r | 0.11066006 |
| Hsd_from_Mmd_52_r | 0.11066006 |
| HS_from_MQbrain_1185_l | 0.113095726 |
| HS_from_MQbrain_29_l | 0.113095726 |
| HS_from_MQbrain_777_r | 0.113095726 |
| HSbrain_1413_l | 0.113095726 |
| HSbrain_440_l | 0.113095726 |
| HSbrain_50_l | 0.113095726 |
| HSbrain_588_l | 0.113095726 |
| Hsd_12_r | 0.113095726 |
| HS_102_l | 0.114595796 |
| HS_38_r | 0.114595796 |
| HS_from_MM_276_l | 0.114595796 |
| HS_from_MQbrain_1164_r | 0.114595796 |
| HS_from_MQbrain_1817_l | 0.114595796 |
| HS_from_MQbrain_184_l | 0.114595796 |
| HS_from_MQbrain_56_l | 0.114595796 |
| HS_from_MQbrain_842_l | 0.114595796 |
| HSbrain_461_r | 0.114595796 |
| HSbrain_621_r | 0.114595796 |
| HSbrain_677_r | 0.114595796 |
| HSbrain_795_r | 0.114595796 |
| HS_from_MM_119_r | 0.115928328 |
| HS_from_MM_331_r | 0.115928328 |
| HS_from_MQbrain_1292_r | 0.115928328 |
| HS_from_MQbrain_1637_r | 0.115928328 |
| HS_from_MQbrain_501_l | 0.115928328 |
| HS_from_MQbrain_609_l | 0.115928328 |
| HS_from_PT_128_l | 0.115928328 |
| HS_from_PT_139_r | 0.115928328 |
| hsa-mir-450-2_r | 0.115928328 |
| HSbrain_1362_r | 0.115928328 |
| HSbrain_26_r | 0.115928328 |
| HSbrain_480_r | 0.115928328 |
| Hsd_from_Mmd_181_r | 0.115928328 |
| HS_246_l | 0.117828853 |
| HS_76_r | 0.117828853 |
| HS_from_MM_157_l | 0.117828853 |
| HS_from_MM_208_r | 0.117828853 |
| HS_from_MM_260_l | 0.117828853 |
| HS_from_MM_34_l | 0.117828853 |
| HS_from_MQbrain_1470_l | 0.117828853 |
| HS_from_MQbrain_273_r | 0.117828853 |
| HS_from_MQbrain_354_r | 0.117828853 |
| HS_from_MQbrain_791_r | 0.117828853 |
| HSbrain_720_l | 0.117828853 |
| HS_from_MM_180_r | 0.119559457 |
| HS_from_MM_184_r | 0.119559457 |
| HS_from_MQbrain_974_l | 0.119559457 |
| HS_from_PT_60_l | 0.119559457 |
| hsa-mir-30b_l | 0.119559457 |
| hsa-mir-491_r | 0.119559457 |
| HSbrain_225_r | 0.119559457 |
| HSbrain_325_r | 0.119559457 |
| HSbrain_441_r | 0.119559457 |
| HSbrain_606_l | 0.119559457 |
| HSbrain_708_l | 0.119559457 |
| Hsd_from_Mmd_23_l | 0.119559457 |
| HS_154_r | 0.121604253 |
| HS_from_MQbrain_161_r | 0.121604253 |
| HS_from_MQbrain_1632_l | 0.121604253 |
| HS_from_MQbrain_1776_r | 0.121604253 |
| HS_from_MQbrain_648_r | 0.121604253 |
| HS_from_MQbrain_940_r | 0.121604253 |
| hsa-mir-548c_l | 0.121604253 |
| hsa-mir-640_l | 0.121604253 |
| HSbrain_305_r | 0.121604253 |
| Hsd_82_r | 0.121604253 |
| Hsd_from_Mmd_136_l | 0.121604253 |
| HS_from_MQbrain_1075_l | 0.12300785 |
| HS_from_MQbrain_1261_r | 0.12300785 |
| HS_from_MQbrain_1412_l | 0.12300785 |
| HS_from_MQbrain_266_l | 0.12300785 |
| HS_from_MQbrain_340_r | 0.12300785 |
| HS_from_MQbrain_760_l | 0.12300785 |
| HS_from_PT_117_r | 0.12300785 |
| hsa-mir-16-1_r | 0.12300785 |
| HSbrain_155_r | 0.12300785 |
| HSbrain_256_r | 0.12300785 |
| HSbrain_402_l | 0.12300785 |
| HSbrain_50_r | 0.12300785 |
| HSbrain_541_r | 0.12300785 |
| Hsd_from_Mmd_314_r | 0.12300785 |
| HS_from_MQbrain_1097_r | 0.124263194 |
| HS_from_MQbrain_1633_r | 0.124263194 |
| HS_from_MQbrain_1806_l | 0.124263194 |
| HS_from_MQbrain_210_r | 0.124263194 |
| HS_from_MQbrain_268_r | 0.124263194 |
| HS_from_MQbrain_619_l | 0.124263194 |
| HS_from_MQbrain_681_l | 0.124263194 |
| HS_from_MQbrain_814_l | 0.124263194 |
| hsa-mir-329-1_r | 0.124263194 |
| hsa-mir-451_l | 0.124263194 |
| hsa-mir-517c_l | 0.124263194 |
| hsa-mir-632_l | 0.124263194 |
| HSbrain_1338_r | 0.124263194 |
| HSbrain_393_l | 0.124263194 |
| Hsd_from_Mmd_154_l | 0.124263194 |
| HS_97_r | 0.126757752 |
| HS_from_MQbrain_1183_l | 0.126757752 |
| HS_from_MQbrain_1675_l | 0.126757752 |
| HS_from_MQbrain_314_l | 0.126757752 |
| HS_from_MQbrain_822_l | 0.126757752 |
| HS_from_MQbrain_897_r | 0.126757752 |
| hsa-mir-299_r | 0.126757752 |
| hsa-mir-616_l | 0.126757752 |
| HSbrain_43_r | 0.126757752 |
| HSbrain_771_l | 0.126757752 |
| HS_290_r | 0.129555956 |
| HS_77_r | 0.129555956 |
| HS_88_r | 0.129555956 |
| HS_from_MQbrain_1102_l | 0.129555956 |
| HS_from_MQbrain_140_l | 0.129555956 |
| HS_from_MQbrain_1413_l | 0.129555956 |
| HS_from_MQbrain_453_l | 0.129555956 |
| HS_from_MQbrain_800_l | 0.129555956 |
| HSbrain_393_r | 0.129555956 |
| HS_50_r | 0.130803986 |
| HS_56_l | 0.130803986 |
| HS_from_MQbrain_1684_r | 0.130803986 |
| HS_from_MQbrain_1808_r | 0.130803986 |
| HS_from_MQbrain_216_l | 0.130803986 |
| HS_from_MQbrain_37_r | 0.130803986 |
| HS_from_MQbrain_672_l | 0.130803986 |
| HS_from_MQbrain_722_l | 0.130803986 |
| HS_from_PT_313_l | 0.130803986 |
| hsa-mir-206_l | 0.130803986 |
| hsa-mir-573_r | 0.130803986 |
| HSbrain_380_r | 0.130803986 |
| HSbrain_776_l | 0.130803986 |
| HSbrain_783_l | 0.130803986 |
| Hsd_from_Mmd_503_l | 0.130803986 |
| Hsd_from_Mmd_94_l | 0.130803986 |
| HS_101_l | 0.132596379 |
| HS_from_MM_200_l | 0.132596379 |
| HS_from_MQbrain_1070_r | 0.132596379 |
| HS_from_MQbrain_1520_l | 0.132596379 |
| HS_from_MQbrain_1845_l | 0.132596379 |
| HS_from_MQbrain_38_r | 0.132596379 |
| HS_from_MQbrain_382_l | 0.132596379 |
| HS_from_MQbrain_715_l | 0.132596379 |
| HS_from_MQbrain_868_l | 0.132596379 |
| HSbrain_12_l | 0.132596379 |
| HSbrain_1364_r | 0.132596379 |
| HSbrain_691_l | 0.132596379 |
| HSbrain_726_l | 0.132596379 |
| Hsd_from_Mmd_155_l | 0.132596379 |
| HS_62_l | 0.134691663 |
| HS_from_MM_102_r | 0.134691663 |
| HS_from_MQbrain_1420_l | 0.134691663 |
| HS_from_MQbrain_1549_l | 0.134691663 |
| hsa-mir-454_r | 0.134691663 |
| hsa-mir-516-1_r | 0.134691663 |
| hsa-mir-520h_l | 0.134691663 |
| HSbrain_220_r | 0.134691663 |
| HSbrain_484_l | 0.134691663 |
| HSbrain_590_l | 0.134691663 |
| HSbrain_636_l | 0.134691663 |
| Hsd_42_r | 0.134691663 |
| Hsd_7_r | 0.134691663 |
| HS_237_r | 0.136637202 |
| HS_289_r | 0.136637202 |
| HS_from_MM_48_l | 0.136637202 |
| HS_from_MQbrain_1725_l | 0.136637202 |
| HS_from_MQbrain_281_r | 0.136637202 |
| HS_from_MQbrain_348_r | 0.136637202 |
| HS_from_MQbrain_600_l | 0.136637202 |
| HS_from_PT_44_l | 0.136637202 |
| HS_from_PT_95_l | 0.136637202 |
| hsa-mir-454_l | 0.136637202 |
| hsa-mir-671_l | 0.136637202 |
| HSbrain_174_r | 0.136637202 |
| HSbrain_222_r | 0.136637202 |
| HSbrain_970_r | 0.136637202 |
| HS_278_l | 0.138217661 |
| HS_91_l | 0.138217661 |
| HS_from_MQbrain_1525_r | 0.138217661 |
| HS_from_MQbrain_161_l | 0.138217661 |
| HS_from_MQbrain_1824_l | 0.138217661 |
| HS_from_MQbrain_1836_l | 0.138217661 |
| HS_from_MQbrain_315_l | 0.138217661 |
| HS_from_MQbrain_697_l | 0.138217661 |
| HS_from_MQbrain_70_l | 0.138217661 |
| hsa-mir-196a-2_r | 0.138217661 |
| hsa-mir-345_r | 0.138217661 |
| hsa-mir-369_l | 0.138217661 |
| hsa-mir-517a_l | 0.138217661 |
| HSbrain_250_l | 0.138217661 |
| HSbrain_753_r | 0.138217661 |
| Hsd_69_l | 0.138217661 |
| HS_277_l | 0.139019426 |
| HS_from_MQbrain_1493_r | 0.139019426 |
| HS_from_MQbrain_1657_r | 0.139019426 |
| HS_from_MQbrain_1756_l | 0.139019426 |
| HS_from_MQbrain_178_r | 0.139019426 |
| HS_from_MQbrain_399_l | 0.139019426 |
| HS_from_MQbrain_804_r | 0.139019426 |
| HS_from_MQbrain_809_l | 0.139019426 |
| HS_from_MQbrain_886_r | 0.139019426 |
| HS_from_MQbrain_90_r | 0.139019426 |
| HSbrain_1377_l | 0.139019426 |
| HSbrain_1416_r | 0.139019426 |
| HSbrain_3_l | 0.139019426 |
| HSbrain_337_r | 0.139019426 |
| HSbrain_509_l | 0.139019426 |
| HSbrain_726_r | 0.139019426 |
| HSbrain_779_r | 0.139019426 |
| Hsd_13_l | 0.139019426 |
| Hsd_32_r | 0.139019426 |
| Hsd_from_Mmd_396_l | 0.139019426 |
| HS_223_r | 0.141416815 |
| HS_39_r | 0.141416815 |
| HS_98_r | 0.141416815 |
| HS_from_MQbrain_1331_r | 0.141416815 |
| HS_from_MQbrain_1417_l | 0.141416815 |
| HS_from_MQbrain_332_l | 0.141416815 |
| HS_from_MQbrain_812_l | 0.141416815 |
| HS_from_MQbrain_845_r | 0.141416815 |
| hsa-mir-328_r | 0.141416815 |
| hsa-mir-488_l | 0.141416815 |
| HSbrain_698_r | 0.141416815 |
| Hsd_from_Mmd_398_r | 0.141416815 |
| Hsd_from_Mmd_487_r | 0.141416815 |
| HS_from_MQbrain_1143_l | 0.14474535 |
| HS_from_MQbrain_770_r | 0.14474535 |
| HS_from_MQbrain_824_r | 0.14474535 |
| HS_from_MQbrain_914_l | 0.14474535 |
| hsa-mir-204_r | 0.14474535 |
| hsa-mir-510_r | 0.14474535 |
| HSbrain_369_r | 0.14474535 |
| HSbrain_82_r | 0.14474535 |
| Hsd_from_Mmd_123_r | 0.14474535 |
| HS_175_l | 0.145370181 |
| HS_207_l | 0.145370181 |
| HS_54_l | 0.145370181 |
| HS_from_MM_288_l | 0.145370181 |
| HS_from_MQbrain_1164_l | 0.145370181 |
| HS_from_MQbrain_1204_r | 0.145370181 |
| HS_from_MQbrain_1227_l | 0.145370181 |
| HS_from_MQbrain_180_r | 0.145370181 |
| HS_from_MQbrain_1810_l | 0.145370181 |
| HS_from_MQbrain_535_r | 0.145370181 |
| HS_from_MQbrain_64_l | 0.145370181 |
| HS_from_PT_283_r | 0.145370181 |
| hsa-mir-30b_r | 0.145370181 |
| hsa-mir-517b_l | 0.145370181 |
| HSbrain_117_r | 0.145370181 |
| HSbrain_139_r | 0.145370181 |
| HSbrain_214_l | 0.145370181 |
| HSbrain_381_l | 0.145370181 |
| HSbrain_620_l | 0.145370181 |
| HSbrain_716_l | 0.145370181 |
| HSbrain_787_l | 0.145370181 |
| Hsd_from_Mmd_82_r | 0.145370181 |
| HS_133_l | 0.146325218 |
| HS_190_r | 0.146325218 |
| HS_281_r | 0.146325218 |
| HS_306_l | 0.146325218 |
| HS_from_MM_136_l | 0.146325218 |
| HS_from_MM_199_l | 0.146325218 |
| HS_from_MQbrain_283_r | 0.146325218 |
| HS_from_MQbrain_429_l | 0.146325218 |
| HS_from_MQbrain_606_r | 0.146325218 |
| HS_from_MQbrain_906_l | 0.146325218 |
| HS_from_MQbrain_973_l | 0.146325218 |
| hsa-mir-217_r | 0.146325218 |
| hsa-mir-368_l | 0.146325218 |
| hsa-mir-520g_l | 0.146325218 |
| hsa-mir-647_l | 0.146325218 |
| hsa-mir-655_l | 0.146325218 |
| HSbrain_307_r | 0.146325218 |
| HSbrain_75_l | 0.146325218 |
| Hsd_from_Mmd_246_r | 0.146325218 |
| Hsd_from_Mmd_500_l | 0.146325218 |
| Hsd_from_Mmd_535_r | 0.146325218 |
| HS_141_l | 0.148196723 |
| HS_286_r | 0.148196723 |
| HS_66_r | 0.148196723 |
| HS_from_MM_118_l | 0.148196723 |
| HS_from_MQbrain_1224_r | 0.148196723 |
| HS_from_MQbrain_1292_l | 0.148196723 |
| HS_from_MQbrain_1817_l | 0.148196723 |
| hsa-mir-365-2_r | 0.148196723 |
| HSbrain_102_r | 0.148196723 |
| HSbrain_244_l | 0.148196723 |
| HSbrain_495_r | 0.148196723 |
| HSbrain_624_l | 0.148196723 |
| HSbrain_627_r | 0.148196723 |
| HSbrain_647_r | 0.148196723 |
| HSbrain_794_r | 0.148196723 |
| Hsd_from_Mmd_501_l | 0.148196723 |
| Hsd_from_Mmd_70_l | 0.148196723 |
| HS_110_r | 0.149149579 |
| HS_139_l | 0.149149579 |
| HS_306_l | 0.149149579 |
| HS_58_l | 0.149149579 |
| HS_from_MQbrain_1088_l | 0.149149579 |
| HS_from_MQbrain_1419_r | 0.149149579 |
| HS_from_MQbrain_1773_l | 0.149149579 |
| HS_from_MQbrain_666_l | 0.149149579 |
| HS_from_MQbrain_817_l | 0.149149579 |
| HS_from_PT_134_l | 0.149149579 |
| HS_from_PT_188_l | 0.149149579 |
| hsa-mir-30c-1_l | 0.149149579 |
| hsa-mir-524_r | 0.149149579 |
| HSbrain_1363_r | 0.149149579 |
| HSbrain_1384_r | 0.149149579 |
| HSbrain_177_r | 0.149149579 |
| HSbrain_198_l | 0.149149579 |
| HSbrain_313_l | 0.149149579 |
| HSbrain_425_l | 0.149149579 |
| HSbrain_622_r | 0.149149579 |
| Hsd_81_r | 0.149149579 |
| Hsd_from_Mmd_516_r | 0.149149579 |
| HS_111_r | 0.152177614 |
| HS_204_r | 0.152177614 |
| HS_45_r | 0.152177614 |
| HS_from_PT_20_r | 0.152177614 |
| hsa-mir-191_r | 0.152177614 |
| HSbrain_304_l | 0.152177614 |
| HSbrain_41_l | 0.152177614 |
| HSbrain_625_l | 0.152177614 |
| HSbrain_717_l | 0.152177614 |
| Hsd_66_l | 0.152177614 |
| Hsd_from_Mmd_261_r | 0.152177614 |
| Hsd_from_Mmd_509_l | 0.152177614 |
| HS_from_MQbrain_1433_r | 0.155479351 |
| HS_from_MQbrain_536_r | 0.155479351 |
| HS_from_MQbrain_629_r | 0.155479351 |
| HS_from_MQbrain_88_l | 0.155479351 |
| hsa-mir-10b_r | 0.155479351 |
| hsa-mir-31_l | 0.155479351 |
| hsa-mir-338_l | 0.155479351 |
| hsa-mir-579_r | 0.155479351 |
| hsa-mir-616_r | 0.155479351 |
| HSbrain_578_r | 0.155479351 |
| Hsd_from_Mmd_424_l | 0.155479351 |
| HS_113_l | 0.15865589 |
| HS_220_l | 0.15865589 |
| HS_from_MQbrain_1168_l | 0.15865589 |
| HS_from_MQbrain_1601_r | 0.15865589 |
| HS_from_MQbrain_686_l | 0.15865589 |
| HS_from_MQbrain_936_l | 0.15865589 |
| HSbrain_431_l | 0.15865589 |
| HSbrain_506_r | 0.15865589 |
| HSbrain_652_l | 0.15865589 |
| HSbrain_673_l | 0.15865589 |
| Hsd_from_Mmd_197_l | 0.15865589 |
| Hsd_from_Mmd_58_r | 0.15865589 |
| HS_167_r | 0.159708794 |
| HS_265_l | 0.159708794 |
| HS_292_r | 0.159708794 |
| HS_from_MQbrain_1110_l | 0.159708794 |
| HS_from_MQbrain_1753_r | 0.159708794 |
| HS_from_MQbrain_1882_r | 0.159708794 |
| HS_from_MQbrain_529_r | 0.159708794 |
| HS_from_MQbrain_604_l | 0.159708794 |
| HS_from_MQbrain_833_l | 0.159708794 |
| HS_from_PT_254_r | 0.159708794 |
| HS_from_PT_91_r | 0.159708794 |
| hsa-mir-382_r | 0.159708794 |
| hsa-mir-516-2_r | 0.159708794 |
| hsa-mir-622_l | 0.159708794 |
| HSbrain_158_l | 0.159708794 |
| HSbrain_202_r | 0.159708794 |
| HSbrain_305_l | 0.159708794 |
| HSbrain_571_l | 0.159708794 |
| HSbrain_644_r | 0.159708794 |
| HSbrain_692_l | 0.159708794 |
| HSbrain_91_r | 0.159708794 |
| Hsd_from_Mmd_347_l | 0.159708794 |
| Hsd_from_Mmd_480_r | 0.159708794 |
| HS_181_l | 0.161647438 |
| HS_288_l | 0.161647438 |
| HS_from_MM_124_l | 0.161647438 |
| HS_from_MQbrain_1215_l | 0.161647438 |
| HS_from_MQbrain_1722_l | 0.161647438 |
| HS_from_MQbrain_1839_l | 0.161647438 |
| HS_from_MQbrain_1877_r | 0.161647438 |
| HS_from_MQbrain_2441_r | 0.161647438 |
| HS_from_MQbrain_587_l | 0.161647438 |
| HS_from_MQbrain_635_l | 0.161647438 |
| HS_from_MQbrain_868_l | 0.161647438 |
| hsa-mir-588_r | 0.161647438 |
| HSbrain_16_l | 0.161647438 |
| HSbrain_218_l | 0.161647438 |
| HSbrain_408_r | 0.161647438 |
| HSbrain_599_r | 0.161647438 |
| HSbrain_663_l | 0.161647438 |
| HSbrain_72_l | 0.161647438 |
| Hsd_106_r | 0.161647438 |
| HS_130_l | 0.16463875 |
| HS_208_r | 0.16463875 |
| HS_from_MQbrain_1229_r | 0.16463875 |
| HS_from_MQbrain_1645_l | 0.16463875 |
| HS_from_MQbrain_288_r | 0.16463875 |
| HS_from_MQbrain_444_r | 0.16463875 |
| HS_from_MQbrain_808_l | 0.16463875 |
| HS_from_PT_41_l | 0.16463875 |
| hsa-mir-296_l | 0.16463875 |
| HSbrain_111_l | 0.16463875 |
| HSbrain_1415_r | 0.16463875 |
| HSbrain_220_l | 0.16463875 |
| HSbrain_366_r | 0.16463875 |
| Hsd_from_Mmd_223_r | 0.16463875 |
| HS_from_MM_311_l | 0.167313007 |
| HS_from_MQbrain_1268_l | 0.167313007 |
| HS_from_MQbrain_1372_l | 0.167313007 |
| HS_from_MQbrain_1376_r | 0.167313007 |
| HS_from_MQbrain_1876_r | 0.167313007 |
| HS_from_MQbrain_2670_r | 0.167313007 |
| HS_from_MQbrain_401_l | 0.167313007 |
| HS_from_MQbrain_42_l | 0.167313007 |
| HS_from_MQbrain_699_r | 0.167313007 |
| HS_from_PT_338_l | 0.167313007 |
| hsa-mir-125b-2_l | 0.167313007 |
| hsa-mir-132_l | 0.167313007 |
| hsa-mir-190_l | 0.167313007 |
| HSbrain_168_l | 0.167313007 |
| HSbrain_513_r | 0.167313007 |
| Hsd_from_Mmd_443_l | 0.167313007 |
| HS_114_l | 0.170255758 |
| HS_61_r | 0.170255758 |
| HS_from_MQbrain_1235_l | 0.170255758 |
| HS_from_MQbrain_1627_r | 0.170255758 |
| HS_from_MQbrain_1667_r | 0.170255758 |
| HS_from_MQbrain_1855_l | 0.170255758 |
| HS_from_MQbrain_482_l | 0.170255758 |
| HS_from_MQbrain_496_r | 0.170255758 |
| HS_from_MQbrain_710_r | 0.170255758 |
| hsa-mir-520f_r | 0.170255758 |
| HSbrain_113_r | 0.170255758 |
| HSbrain_626_r | 0.170255758 |
| HSbrain_85_r | 0.170255758 |
| Hsd_from_Mmd_107_r | 0.170255758 |
| Hsd_from_Mmd_176_r | 0.170255758 |
| HS_29_l | 0.173467438 |
| HS_from_MM_6_r | 0.173467438 |
| HS_from_MQbrain_560_r | 0.173467438 |
| HS_from_MQbrain_57_l | 0.173467438 |
| hsa-mir-643_r | 0.173467438 |
| HSbrain_157_l | 0.173467438 |
| HSbrain_336_l | 0.173467438 |
| HSbrain_483_r | 0.173467438 |
| HSbrain_614_l | 0.173467438 |
| HSbrain_777_r | 0.173467438 |
| Hsd_80_r | 0.173467438 |
| Hsd_from_Mmd_35_r | 0.173467438 |
| Hsd_from_Mmd_370_r | 0.173467438 |
| Hsd_from_Mmd_97_l | 0.173467438 |
| HS_191_r | 0.175200251 |
| HS_231_l | 0.175200251 |
| HS_from_MQbrain_1091_l | 0.175200251 |
| HS_from_MQbrain_1463_l | 0.175200251 |
| HS_from_MQbrain_1556_r | 0.175200251 |
| HS_from_MQbrain_1708_l | 0.175200251 |
| HS_from_MQbrain_1752_l | 0.175200251 |
| HS_from_MQbrain_1777_l | 0.175200251 |
| HS_from_MQbrain_437_l | 0.175200251 |
| HS_from_MQbrain_555_r | 0.175200251 |
| HS_from_MQbrain_734_l | 0.175200251 |
| HS_from_MQbrain_85_l | 0.175200251 |
| HS_from_PT_115_r | 0.175200251 |
| HS_from_PT_34_r | 0.175200251 |
| hsa-mir-770_r | 0.175200251 |
| HSbrain_429_l | 0.175200251 |
| HSbrain_540_l | 0.175200251 |
| HSbrain_643_l | 0.175200251 |
| HSbrain_687_l | 0.175200251 |
| HSbrain_916_l | 0.175200251 |
| HSbrain_970_l | 0.175200251 |
| Hsd_from_Mmd_527_l | 0.175200251 |
| HS_109_r | 0.177405071 |
| HS_217_l | 0.177405071 |
| HS_from_MQbrain_1126_l | 0.177405071 |
| HS_from_MQbrain_1323_l | 0.177405071 |
| HS_from_MQbrain_1442_l | 0.177405071 |
| HS_from_MQbrain_1470_r | 0.177405071 |
| HS_from_MQbrain_386_r | 0.177405071 |
| HS_from_PT_190_r | 0.177405071 |
| HS_from_PT_211_r | 0.177405071 |
| hsa-mir-374_l | 0.177405071 |
| HSbrain_1414_r | 0.177405071 |
| HSbrain_347_l | 0.177405071 |
| HSbrain_568_r | 0.177405071 |
| HSbrain_690_l | 0.177405071 |
| HSbrain_692_r | 0.177405071 |
| HSbrain_744_l | 0.177405071 |
| HSbrain_794_l | 0.177405071 |
| Hsd_from_Mmd_167_r | 0.177405071 |
| Hsd_from_Mmd_42_r | 0.177405071 |
| Hsd_from_Mmd_480_l | 0.177405071 |
| HS_266_r | 0.180260145 |
| HS_285_r | 0.180260145 |
| HS_292_r | 0.180260145 |
| HS_47_l | 0.180260145 |
| HS_from_MQbrain_1110_r | 0.180260145 |
| HS_from_MQbrain_1631_r | 0.180260145 |
| HS_from_MQbrain_550_l | 0.180260145 |
| HS_from_MQbrain_661_r | 0.180260145 |
| HS_from_MQbrain_991_r | 0.180260145 |
| hsa-mir-365-1_l | 0.180260145 |
| HSbrain_1331_r | 0.180260145 |
| HSbrain_180_l | 0.180260145 |
| HSbrain_263_r | 0.180260145 |
| HSbrain_593_r | 0.180260145 |
| HSbrain_689_r | 0.180260145 |
| Hsd_from_Mmd_217_r | 0.180260145 |
| Hsd_from_Mmd_286_r | 0.180260145 |
| HS_138_l | 0.184337179 |
| HS_177_l | 0.184337179 |
| HS_182_r | 0.184337179 |
| HS_243_l | 0.184337179 |
| HS_from_MM_131_l | 0.184337179 |
| HS_from_MQbrain_1227_l | 0.184337179 |
| HS_from_MQbrain_188_l | 0.184337179 |
| HSbrain_621_l | 0.184337179 |
| HSbrain_778_l | 0.184337179 |
| Hsd_84_r | 0.184337179 |
| Hsd_from_Mmd_120_r | 0.184337179 |
| HS_142_l | 0.185815478 |
| HS_303_r | 0.185815478 |
| HS_77_l | 0.185815478 |
| HS_from_MM_256_r | 0.185815478 |
| HS_from_MM_310_l | 0.185815478 |
| HS_from_MQbrain_111_l | 0.185815478 |
| HS_from_MQbrain_1354_r | 0.185815478 |
| HS_from_MQbrain_1394_l | 0.185815478 |
| HS_from_MQbrain_17_r | 0.185815478 |
| HS_from_MQbrain_1778_r | 0.185815478 |
| HS_from_MQbrain_22_l | 0.185815478 |
| HS_from_MQbrain_272_l | 0.185815478 |
| HS_from_MQbrain_453_r | 0.185815478 |
| HS_from_MQbrain_914_l | 0.185815478 |
| HS_from_PT_157_r | 0.185815478 |
| hsa-mir-135a-2_r | 0.185815478 |
| hsa-mir-557_l | 0.185815478 |
| hsa-mir-593_r | 0.185815478 |
| HSbrain_1364_l | 0.185815478 |
| HSbrain_166_l | 0.185815478 |
| HSbrain_32_l | 0.185815478 |
| HSbrain_662_r | 0.185815478 |
| HSbrain_80_l | 0.185815478 |
| Hsd_8_r | 0.185815478 |
| Hsd_from_Mmd_48_r | 0.185815478 |
| HS_189_l | 0.188706417 |
| HS_241_r | 0.188706417 |
| HS_50_l | 0.188706417 |
| HS_from_MQbrain_1346_r | 0.188706417 |
| HS_from_MQbrain_194_l | 0.188706417 |
| HS_from_MQbrain_36_r | 0.188706417 |
| HS_from_MQbrain_691_r | 0.188706417 |
| HS_from_MQbrain_745_r | 0.188706417 |
| HS_from_MQbrain_859_l | 0.188706417 |
| HS_from_PT_126_l | 0.188706417 |
| hsa-mir-367_r | 0.188706417 |
| hsa-mir-518e_r | 0.188706417 |
| HSbrain_141_r | 0.188706417 |
| HSbrain_270_r | 0.188706417 |
| HSbrain_497_l | 0.188706417 |
| HSbrain_570_l | 0.188706417 |
| Hsd_96_r | 0.188706417 |
| Hsd_from_Mmd_471_r | 0.188706417 |
| HS_105_l | 0.190550803 |
| HS_166_l | 0.190550803 |
| HS_from_MM_80_l | 0.190550803 |
| HS_from_MQbrain_1300_r | 0.190550803 |
| HS_from_MQbrain_1347_r | 0.190550803 |
| HS_from_MQbrain_1365_l | 0.190550803 |
| HS_from_MQbrain_1437_l | 0.190550803 |
| HS_from_MQbrain_397_l | 0.190550803 |
| HS_from_MQbrain_405_r | 0.190550803 |
| HS_from_MQbrain_626_r | 0.190550803 |
| HS_from_MQbrain_653_l | 0.190550803 |
| HS_from_MQbrain_675_l | 0.190550803 |
| HS_from_MQbrain_819_r | 0.190550803 |
| HS_from_MQbrain_968_r | 0.190550803 |
| HS_from_MQbrain_97_r | 0.190550803 |
| HS_from_PT_238_l | 0.190550803 |
| HS_from_PT_27_l | 0.190550803 |
| hsa-mir-222_r | 0.190550803 |
| hsa-mir-502_l | 0.190550803 |
| HSbrain_809_r | 0.190550803 |
| Hsd_62_l | 0.190550803 |
| Hsd_95_l | 0.190550803 |
| Hsd_from_Mmd_163_r | 0.190550803 |
| Hsd_from_Mmd_253_l | 0.190550803 |
| HS_from_MM_198_r | 0.194339793 |
| HS_from_MM_35_r | 0.194339793 |
| HS_from_MQbrain_1272_l | 0.194339793 |
| HS_from_MQbrain_1421_l | 0.194339793 |
| HS_from_MQbrain_375_l | 0.194339793 |
| HS_from_MQbrain_55_l | 0.194339793 |
| HS_from_PT_149_r | 0.194339793 |
| hsa-mir-550-2_r | 0.194339793 |
| hsa-mir-623_r | 0.194339793 |
| hsa-mir-648_l | 0.194339793 |
| HSbrain_259_r | 0.194339793 |
| HSbrain_755_l | 0.194339793 |
| Hsd_from_Mmd_530_l | 0.194339793 |
| Hsd_from_Mmd_81_r | 0.194339793 |
| HS_from_MM_190_l | 0.197457594 |
| HS_from_MQbrain_1159_l | 0.197457594 |
| HS_from_MQbrain_1514_r | 0.197457594 |
| HS_from_MQbrain_467_l | 0.197457594 |
| HS_from_MQbrain_500_r | 0.197457594 |
| HS_from_MQbrain_635_r | 0.197457594 |
| HS_from_MQbrain_789_r | 0.197457594 |
| HS_from_MQbrain_968_l | 0.197457594 |
| hsa-mir-204_l | 0.197457594 |
| hsa-mir-219-1_r | 0.197457594 |
| HSbrain_1337_l | 0.197457594 |
| HSbrain_190_r | 0.197457594 |
| HSbrain_210_l | 0.197457594 |
| HSbrain_289_r | 0.197457594 |
| HSbrain_479_l | 0.197457594 |
| HSbrain_755_r | 0.197457594 |
| Hsd_24_r | 0.197457594 |
| Hsd_from_Mmd_178_l | 0.197457594 |
| HS_from_MQbrain_1100_r | 0.199721903 |
| HS_from_MQbrain_1210_l | 0.199721903 |
| HS_from_MQbrain_1250_l | 0.199721903 |
| HS_from_MQbrain_1357_l | 0.199721903 |
| HS_from_MQbrain_1405_l | 0.199721903 |
| HS_from_MQbrain_1415_r | 0.199721903 |
| HS_from_MQbrain_1517_r | 0.199721903 |
| HS_from_MQbrain_1747_l | 0.199721903 |
| HS_from_MQbrain_212_r | 0.199721903 |
| HS_from_MQbrain_229_l | 0.199721903 |
| HS_from_MQbrain_235_r | 0.199721903 |
| HS_from_MQbrain_280_l | 0.199721903 |
| hsa-mir-138-2_l | 0.199721903 |
| hsa-mir-524_l | 0.199721903 |
| hsa-mir-568_l | 0.199721903 |
| hsa-mir-95_r | 0.199721903 |
| HSbrain_22_l | 0.199721903 |
| HSbrain_388_r | 0.199721903 |
| HSbrain_482_l | 0.199721903 |
| HSbrain_588_r | 0.199721903 |
| HSbrain_651_r | 0.199721903 |
| Hsd_41_r | 0.199721903 |
| Hsd_from_Mmd_478_l | 0.199721903 |

¹Wilcoxon analysis: cut off adjusted p value < 0.2

TABLE 4b
Up- or downregulated small RNAs adenocarcinoma compared to
adenoma

| | adenoma/carcinoma | carcinoma | adenoma | carcinoma/adenoma |
|---|---|---|---|---|
| SystematicName | (adj p)¹ | (avg expression) | (avg expression) | (ratio expression)² |
| hsa-mir-375_r | 0.0393 | −0.1480 | 0.8295 | −0.9775 |
| HS_from_MQbrain_1985_l | 0.0474 | 0.3803 | 1.1112 | −0.7308 |
| HS_from_MQbrain_579_l | 0.0660 | 0.3918 | 1.1126 | −0.7208 |
| HS_from_MQbrain_1958_l | 0.0723 | 0.3762 | 1.0830 | −0.7067 |
| HS_from_MQbrain_1235_l | 0.1703 | 1.1233 | 1.7573 | −0.6340 |
| HS_from_MQbrain_1323_l | 0.1774 | 1.1608 | 1.7828 | −0.6220 |
| HS_from_MQbrain_1525_r | 0.1382 | 0.3320 | 0.9272 | −0.5951 |
| HS_from_MQbrain_972_r | 0.0772 | 0.2041 | 0.7900 | −0.5859 |
| HSbrain_636_l | 0.1347 | 0.8158 | 1.3298 | −0.5141 |
| HS_266_r | 0.1803 | 0.8889 | 1.3712 | −0.4823 |
| HS_from_PT_13_l | 0.0883 | −0.9932 | −0.5123 | −0.4809 |
| HS_from_MQbrain_529_r | 0.1597 | 0.0776 | 0.5481 | −0.4705 |
| HS_from_MQbrain_470_l | 0.0750 | 0.8272 | 1.2930 | −0.4658 |
| hsa-mir-10b_r | 0.1555 | −0.0924 | 0.3721 | −0.4645 |
| Hsd_from_Mmd_166_l | 0.0501 | −0.8357 | −0.3751 | −0.4607 |
| Hsd_from_Mmd_136_l | 0.1216 | −1.2261 | −0.7690 | −0.4571 |
| hsa-mir-451_l | 0.3459 | −0.4469 | 0.0020 | −0.4489 |
| Hsd_from_Mmd_524_l | 0.0995 | −0.9078 | −0.4694 | −0.4385 |
| HSbrain_773_r | 0.0928 | −1.1665 | −0.7301 | −0.4364 |
| HS_from_MQbrain_828_l | 0.0320 | 0.2589 | 0.6920 | −0.4331 |
| hsa-mir-190_l | 0.1673 | −1.4076 | −0.9760 | −0.4316 |
| HS_from_MM_6_r | 0.1735 | 0.4691 | 0.9005 | −0.4314 |
| hsa-mir-573_r | 0.1308 | −1.2345 | −0.8053 | −0.4291 |
| hsa-mir-193a_l | 0.0995 | 0.6922 | 1.1195 | −0.4273 |
| HS_from_MM_84_l | 0.1010 | −0.6664 | −0.2459 | −0.4205 |
| HS_from_PT_190_r | 0.1774 | 1.1254 | 1.5446 | −0.4192 |
| Hsd_from_Mmd_123_r | 0.1447 | 0.5308 | 0.9486 | −0.4178 |
| HS_151_r | 0.0913 | −0.9626 | −0.5484 | −0.4141 |
| HSbrain_1384_r | 0.1491 | 0.1926 | 0.6048 | −0.4122 |
| hsa-mir-368_l | 0.1463 | −0.1578 | 0.2436 | −0.4014 |
| hsa-mir-520f_r | 0.1703 | −0.6603 | −0.2591 | −0.4012 |
| HS_from_MQbrain_1229_r | 0.1646 | −0.0990 | 0.3017 | −0.4007 |
| HSbrain_276_l | 0.0393 | −0.2953 | 0.1014 | −0.3967 |
| HS_from_MQbrain_1417_l | 0.1414 | 0.3389 | 0.7346 | −0.3957 |
| HSbrain_220_l | 0.1646 | −0.2992 | 0.0905 | −0.3897 |
| HS_from_MM_136_l | 0.1463 | −1.0881 | −0.7064 | −0.3816 |
| hsa-mir-450-2_r | 0.1159 | −0.4279 | −0.0493 | −0.3785 |
| HSbrain_916_l | 0.1752 | 1.4491 | 1.8251 | −0.3760 |
| hsa-mir-516-1_r | 0.1347 | −0.9059 | −0.5366 | −0.3693 |
| HS_124_r | 0.0249 | −0.2279 | 0.1382 | −0.3661 |
| HSbrain_776_l | 0.1308 | 1.1157 | 1.4804 | −0.3647 |
| Hsd_from_Mmd_107_r | 0.1703 | −1.2270 | −0.8628 | −0.3643 |
| HS_from_MQbrain_1771_r | 0.0913 | 0.1028 | 0.4671 | −0.3643 |
| hsa-mir-367_r | 0.1887 | −0.2475 | 0.1160 | −0.3636 |
| HSbrain_52_r | 0.0983 | 0.4883 | 0.8497 | −0.3614 |
| HSbrain_399_l | 0.0320 | −0.0904 | 0.2705 | −0.3609 |
| HS_from_MQbrain_1901_r | 0.0946 | −0.4682 | −0.1080 | −0.3602 |
| HSbrain_259_r | 0.1943 | 0.3494 | 0.7076 | −0.3581 |
| hsa-mir-548b_l | 0.0339 | −0.3487 | 0.0094 | −0.3581 |
| hsa-mir-454_r | 0.1347 | −0.6903 | −0.3370 | −0.3534 |
| HSbrain_570_l | 0.1887 | 0.1544 | 0.5074 | −0.3530 |
| HS_from_MQbrain_111_l | 0.1858 | −0.2182 | 0.1331 | −0.3513 |
| HS_307_r | 0.0750 | −0.8051 | −0.4560 | −0.3491 |
| HSbrain_538_r | 0.0552 | −0.0160 | 0.3321 | −0.3481 |
| HSbrain_513_r | 0.1673 | −0.6112 | −0.2644 | −0.3469 |
| Hsd_81_r | 0.1491 | −0.6940 | −0.3476 | −0.3465 |
| HS_from_MQbrain_1485_l | 0.0292 | −0.2157 | 0.1282 | −0.3439 |
| hsa-mir-648_l | 0.1943 | 0.4250 | 0.7676 | −0.3427 |
| hsa-mir-331_l | 0.0347 | −0.4701 | −0.1282 | −0.3419 |
| HS_from_MQbrain_951_l | 0.1078 | 0.5785 | 0.9191 | −0.3405 |
| HSbrain_72_l | 0.1616 | 1.0011 | 1.3415 | −0.3405 |
| HS_from_MQbrain_315_l | 0.1382 | 0.1929 | 0.5309 | −0.3381 |
| HS_from_MM_180_r | 0.1196 | −0.6729 | −0.3357 | −0.3372 |
| HS_from_PT_20_r | 0.1522 | 0.3244 | 0.6602 | −0.3358 |
| Hsd_from_Mmd_48_r | 0.1858 | −0.3148 | 0.0209 | −0.3358 |
| hsa-mir-433_r | 0.0772 | −0.1074 | 0.2280 | −0.3354 |
| hsa-mir-516-2_r | 0.1597 | −0.8140 | −0.4802 | −0.3337 |
| HS_from_MQbrain_441_l | 0.4342 | −0.0869 | 0.2455 | −0.3324 |
| HSbrain_117_r | 0.1454 | 0.0411 | 0.3731 | −0.3321 |
| HS_from_MQbrain_330_r | 0.0857 | 0.3556 | 0.6874 | −0.3318 |
| hsa-mir-299_r | 0.1268 | 0.3466 | 0.6783 | −0.3317 |
| HS_from_MQbrain_1493_r | 0.1390 | 1.0831 | 1.4142 | −0.3310 |
| HS_from_MQbrain_1413_l | 0.1296 | 0.0572 | 0.3880 | −0.3308 |
| hsa-mir-548c_l | 0.1216 | −0.6506 | −0.3199 | −0.3307 |
| HSbrain_420_r | 0.0583 | 0.1546 | 0.4847 | −0.3301 |
| HSbrain_6_r | 0.0764 | 0.2574 | 0.5872 | −0.3298 |
| HS_from_MM_295_l | 0.0205 | −0.1367 | 0.1924 | −0.3291 |
| HS_from_MQbrain_2670_r | 0.1673 | 0.3448 | 0.6730 | −0.3282 |
| HSbrain_716_l | 0.1454 | 0.1785 | 0.5044 | −0.3260 |
| HS_from_PT_282_l | 0.0913 | −0.0644 | 0.2604 | −0.3248 |
| Hsd_from_Mmd_314_r | 0.1230 | −0.6381 | −0.3153 | −0.3227 |
| HS_from_MQbrain_468_r | 0.0857 | 0.0111 | 0.3325 | −0.3213 |
| HS_from_MQbrain_501_l | 0.1159 | 0.1924 | 0.5136 | −0.3212 |
| HSbrain_369_r | 0.1447 | −0.1235 | 0.1952 | −0.3188 |
| hsa-mir-653_l | 0.0282 | −0.2258 | 0.0912 | −0.3170 |
| HS_118_l | 0.3927 | 0.4518 | 0.7665 | −0.3147 |
| hsa-mir-652_r | 0.0849 | −0.5348 | −0.2206 | −0.3142 |
| HS_from_MM_310_l | 0.1858 | −0.5347 | −0.2217 | −0.3130 |
| HSbrain_95_l | 0.0849 | −0.3123 | 0.0005 | −0.3128 |
| HS_from_MQbrain_29_l | 0.1131 | 0.4126 | 0.7253 | −0.3127 |
| HSbrain_174_r | 0.1366 | −0.7406 | −0.4286 | −0.3120 |
| Hsd_7_r | 0.1347 | −0.0496 | 0.2607 | −0.3103 |
| hsa-mir-524_r | 0.1491 | −0.0033 | 0.3055 | −0.3087 |
| hsa-mir-412_l | 0.0350 | −0.1137 | 0.1947 | −0.3085 |
| HS_177_l | 0.1843 | −0.2397 | 0.0685 | −0.3082 |
| HS_from_MM_342_r | 0.0772 | −0.0065 | 0.3015 | −0.3080 |
| HSbrain_970_r | 0.1366 | −0.6926 | −0.3850 | −0.3076 |
| HS_from_PT_187_r | 0.0327 | −0.1851 | 0.1217 | −0.3068 |
| HS_from_MQbrain_1227_l | 0.1843 | −0.0475 | 0.2585 | −0.3059 |
| HS_from_MQbrain_97_r | 0.1906 | 0.2065 | 0.5122 | −0.3058 |
| HS_from_MQbrain_1756_l | 0.1390 | 0.2193 | 0.5248 | −0.3055 |
| hsa-let-7c_r | 0.0501 | −0.1593 | 0.1458 | −0.3051 |
| Hsd_from_Mmd_82_r | 0.1454 | −0.5385 | −0.2337 | −0.3048 |
| hsa-mir-624_l | 0.0895 | −0.4583 | −0.1544 | −0.3039 |
| HS_from_MQbrain_1183_l | 0.1268 | 0.9448 | 1.2486 | −0.3038 |
| HS_from_MM_72_r | 0.0913 | −0.3145 | −0.0123 | −0.3022 |
| hsa-mir-497_r | 0.0582 | 0.0098 | 0.3116 | −0.3018 |
| HS_207_l | 0.1454 | −0.7159 | −0.4144 | −0.3016 |
| HS_from_MQbrain_1297_l | 0.0205 | 0.1527 | 0.4538 | −0.3012 |
| HSbrain_794_r | 0.1482 | 0.4585 | 0.7581 | −0.2996 |
| HS_from_PT_254_r | 0.1597 | 0.7318 | 1.0312 | −0.2994 |
| HS_from_MM_256_r | 0.1858 | −0.8368 | −0.5378 | −0.2990 |
| Hsd_from_Mmd_225_l | 0.0928 | −0.5076 | −0.2092 | −0.2984 |
| HS_from_MQbrain_1363_l | 0.0252 | 0.0001 | 0.2974 | −0.2973 |
| HS_277_l | 0.1390 | −0.3910 | −0.0947 | −0.2963 |
| HS_from_MQbrain_1227_l | 0.1843 | −0.2622 | 0.0336 | −0.2958 |
| Hsd_96_r | 0.1887 | −0.5046 | −0.2090 | −0.2956 |
| HS_79_l | 0.0482 | −0.0825 | 0.2126 | −0.2951 |
| HS_from_MQbrain_664_r | 0.0706 | 0.0091 | 0.3041 | −0.2950 |
| HSbrain_796_r | 0.0277 | −0.1402 | 0.1547 | −0.2949 |
| Hsd_18_r | 0.0393 | −0.1922 | 0.1024 | −0.2946 |
| HS_292_r | 0.1803 | −0.6801 | −0.3855 | −0.2946 |
| HSbrain_624_l | 0.1482 | 0.1118 | 0.4059 | −0.2941 |
| HS_from_MQbrain_1097_r | 0.1243 | 0.3204 | 0.6142 | −0.2939 |
| hsa-mir-190_r | 0.0764 | −0.5001 | −0.2067 | −0.2934 |
| HS_from_MQbrain_180_r | 0.1454 | 0.0609 | 0.3529 | −0.2920 |
| HS_189_l | 0.1887 | 0.5895 | 0.8806 | −0.2911 |
| HSbrain_381_l | 0.1454 | −0.8145 | −0.5240 | −0.2905 |
| HSbrain_1076_r | 0.0660 | −0.1027 | 0.1878 | −0.2905 |
| hsa-mir-299_l | 0.0772 | −0.1635 | 0.1264 | −0.2899 |
| HSbrain_388_r | 0.1997 | −0.1095 | 0.1800 | −0.2896 |
| HS_from_MQbrain_1781_l | 0.0369 | −0.2509 | 0.0384 | −0.2893 |
| HS_from_MM_232_r | 0.0727 | −0.4188 | −0.1303 | −0.2886 |
| HSbrain_1331_r | 0.1803 | −0.0587 | 0.2288 | −0.2875 |
| hsa-mir-633_r | 0.1045 | −0.4375 | −0.1504 | −0.2870 |
| HS_246_l | 0.1178 | −0.4272 | −0.1410 | −0.2862 |
| Hsd_42_r | 0.1347 | 0.1048 | 0.3903 | −0.2855 |
| hsa-mir-767_r | 0.0239 | −0.2413 | 0.0431 | −0.2845 |
| HS_292_r | 0.1803 | −0.6740 | −0.3896 | −0.2844 |
| HS_from_MQbrain_154_r | 0.0772 | −0.0165 | 0.2678 | −0.2844 |
| HS_from_MM_34_l | 0.3286 | −0.2129 | 0.0705 | −0.2833 |
| HSbrain_507_l | 0.1026 | −0.2025 | 0.0806 | −0.2831 |
| hsa-mir-130b_l | 0.0750 | −0.4336 | −0.1518 | −0.2818 |
| HS_from_MQbrain_1300_r | 0.1906 | −0.3783 | −0.0970 | −0.2812 |
| Hsd_from_Mmd_157_r | 0.0264 | −0.1988 | 0.0821 | −0.2808 |
| hsa-mir-1-1_l | 0.0750 | −0.3136 | −0.0329 | −0.2807 |
| Hsd_from_Mmd_58_r | 0.1587 | −0.8765 | −0.5960 | −0.2805 |
| HS_from_MQbrain_194_l | 0.1887 | 0.7959 | 1.0759 | −0.2801 |
| HS_213_l | 0.0482 | −0.1424 | 0.1376 | −0.2799 |
| HS_from_MM_32_r | 0.0750 | −0.1992 | 0.0806 | −0.2797 |
| HS_from_MQbrain_74_r | 0.0749 | 0.4392 | 0.7181 | −0.2789 |
| Hsd_46_l | 0.0913 | 0.0904 | 0.3671 | −0.2768 |
| HS_from_PT_41_l | 0.1646 | 0.0162 | 0.2925 | −0.2764 |
| HS_from_MM_208_r | 0.1178 | 0.0831 | 0.3590 | −0.2759 |
| HS_212_l | 0.0501 | −0.2004 | 0.0752 | −0.2756 |
| HSbrain_482_l | 0.8056 | 0.1051 | 0.3806 | −0.2754 |
| HS_166_l | 0.1906 | 0.9732 | 1.2478 | −0.2746 |
| HS_288_l | 0.1616 | 0.6490 | 0.9235 | −0.2744 |
| HS_from_MM_157_l | 0.1178 | −0.5670 | −0.2926 | −0.2744 |
| hsa-mir-206_l | 0.1308 | −0.5186 | −0.2448 | −0.2738 |
| HS_from_PT_101_r | 0.0518 | −0.1057 | 0.1677 | −0.2735 |
| Hsd_108_l | 0.0474 | −0.0852 | 0.1879 | −0.2730 |
| HSbrain_532_l | 0.0961 | −0.1027 | 0.1698 | −0.2725 |
| HS_114_l | 0.4078 | 0.5279 | 0.8004 | −0.2725 |
| HS_from_MM_18_l | 0.0849 | −0.0504 | 0.2213 | −0.2717 |
| HSbrain_361_l | 0.0807 | 0.2281 | 0.4996 | −0.2715 |
| HS_from_MQbrain_1700_r | 0.1010 | 0.0114 | 0.2793 | −0.2679 |
| HS_from_MQbrain_1877_r | 0.1616 | 0.7927 | 1.0605 | −0.2679 |
| HSbrain_614_l | 0.1735 | −0.0743 | 0.1918 | −0.2661 |
| HS_from_MQbrain_824_r | 0.1447 | −0.0312 | 0.2344 | −0.2656 |
| hsa-mir-616_l | 0.1268 | −0.2425 | 0.0224 | −0.2650 |
| Hsd_from_Mmd_44_r | 0.0849 | −0.3018 | −0.0377 | −0.2641 |
| HS_from_MQbrain_88_l | 0.1555 | −0.6048 | −0.3408 | −0.2639 |
| HS_from_MQbrain_1717_r | 0.0961 | −0.1303 | 0.1329 | −0.2632 |
| HS_29_l | 0.8454 | −0.5409 | −0.2781 | −0.2629 |
| Hsd_from_Mmd_49_l | 0.0583 | 0.1113 | 0.3738 | −0.2625 |
| HS_from_PT_237_r | 0.0723 | 0.1130 | 0.3753 | −0.2623 |
| HSbrain_41_l | 0.1522 | 0.4515 | 0.7137 | −0.2622 |
| HSbrain_811_l | 0.0260 | −0.0122 | 0.2490 | −0.2611 |
| HS_219_l | 0.0750 | 0.0321 | 0.2931 | −0.2610 |
| HS_from_MQbrain_672_l | 0.1308 | 0.2914 | 0.5518 | −0.2604 |
| HSbrain_419_l | 0.0749 | −0.0824 | 0.1776 | −0.2600 |
| HSbrain_55_l | 0.0537 | −0.0575 | 0.2025 | −0.2600 |
| HS_from_MM_331_r | 0.1159 | −0.1726 | 0.0869 | −0.2594 |
| HS_from_MQbrain_85_l | 0.1752 | 0.7506 | 1.0100 | −0.2594 |
| HS_from_MQbrain_900_r | 0.1060 | −0.2078 | 0.0506 | −0.2584 |
| HS_from_MQbrain_854_r | 0.0446 | −0.0875 | 0.1696 | −0.2571 |
| HS_from_MQbrain_210_r | 0.1243 | −0.1029 | 0.1535 | −0.2564 |
| Hsd_from_Mmd_422_l | 0.0928 | −0.1881 | 0.0673 | −0.2554 |
| HS_from_PT_149_r | 0.1943 | −0.5868 | −0.3316 | −0.2552 |
| hsa-mir-593_r | 0.1858 | −0.2304 | 0.0240 | −0.2545 |
| HS_from_MQbrain_833_l | 0.1597 | −0.5419 | −0.2877 | −0.2542 |
| Hsd_from_Mmd_167_r | 0.1774 | 0.1417 | 0.3959 | −0.2541 |
| HS_from_MQbrain_1374_r | 0.0838 | −0.2929 | −0.0392 | −0.2536 |
| HS_from_MQbrain_791_r | 0.1178 | −0.7234 | −0.4697 | −0.2536 |
| HS_281_r | 0.1463 | −0.1708 | 0.0827 | −0.2536 |
| HS_from_MQbrain_555_r | 0.1752 | 0.0272 | 0.2808 | −0.2535 |
| HS_from_MQbrain_1126_l | 0.1774 | −0.6334 | −0.3799 | −0.2535 |
| HS_from_MQbrain_90_r | 0.1390 | −0.2479 | 0.0050 | −0.2529 |
| HS_17_l | 0.0537 | −0.0256 | 0.2273 | −0.2529 |
| HS_from_MQbrain_120_l | 0.0686 | −0.0417 | 0.2098 | −0.2515 |
| hsa-mir-30c-1_l | 0.1491 | −0.2525 | −0.0024 | −0.2500 |
| HS_from_MQbrain_1159_l | 0.1975 | 0.4929 | 0.7421 | −0.2491 |
| hsa-mir-647_l | 0.1463 | −0.0654 | 0.1833 | −0.2487 |
| HS_from_MQbrain_1212_l | 0.0498 | −0.2370 | 0.0116 | −0.2486 |
| Hsd_from_Mmd_166_r | 0.1060 | −0.4404 | −0.1919 | −0.2485 |
| HS_from_MQbrain_42_l | 0.1673 | −0.1400 | 0.1078 | −0.2478 |
| hsa-mir-382_r | 0.1597 | −0.2563 | −0.0087 | −0.2476 |
| HS_from_MM_184_r | 0.1196 | −0.1915 | 0.0560 | −0.2475 |
| Hsd_from_Mmd_478_l | 0.1997 | 0.0790 | 0.3263 | −0.2474 |
| HSbrain_1415_r | 0.1646 | −0.7870 | −0.5399 | −0.2471 |
| HS_141_l | 0.1482 | 0.8423 | 1.0869 | −0.2446 |
| HS_from_MQbrain_1740_r | 0.0750 | 0.1451 | 0.3893 | −0.2442 |
| hsa-let-7f-1_r | 0.1060 | −0.2858 | −0.0417 | −0.2440 |
| HSbrain_180_l | 0.1803 | −0.1130 | 0.1309 | −0.2440 |
| HS_from_MQbrain_609_l | 0.1159 | 0.4251 | 0.6690 | −0.2439 |
| Hsd_from_Mmd_302_r | 0.0849 | −0.0855 | 0.1577 | −0.2432 |
| HSbrain_1364_l | 0.1858 | 0.6922 | 0.9342 | −0.2420 |
| HSbrain_425_l | 0.1491 | −0.3661 | −0.1243 | −0.2418 |
| Hsd_from_Mmd_261_r | 0.1522 | −0.3958 | −0.1542 | −0.2416 |
| HS_from_MQbrain_1725_l | 0.1366 | −0.2059 | 0.0357 | −0.2416 |
| HS_from_MQbrain_542_r | 0.0482 | 0.0882 | 0.3297 | −0.2416 |
| Hsd_from_Mmd_163_r | 0.1906 | 0.0070 | 0.2484 | −0.2414 |
| hsa-mir-98_r | 0.0750 | −0.3063 | −0.0650 | −0.2413 |
| HS_from_MQbrain_812_l | 0.1414 | −0.7725 | −0.5315 | −0.2410 |
| HS_from_MQbrain_209_r | 0.0660 | −0.0235 | 0.2170 | −0.2406 |
| HSbrain_250_r | 0.0772 | −0.0569 | 0.1834 | −0.2403 |
| Hsd_70_r | 0.0350 | −0.2673 | −0.0273 | −0.2400 |
| HSbrain_272_l | 0.0393 | −0.2116 | 0.0282 | −0.2398 |
| HSbrain_473_l | 0.0807 | −0.1738 | 0.0656 | −0.2394 |
| HS_from_MQbrain_936_l | 0.1587 | −0.4116 | −0.1725 | −0.2391 |
| HSbrain_50_r | 0.1230 | −0.2109 | 0.0273 | −0.2383 |
| HS_from_MQbrain_619_l | 0.1243 | −0.1904 | 0.0476 | −0.2380 |
| hsa-mir-502_l | 0.1906 | −0.5042 | −0.2665 | −0.2377 |
| Hsd_from_Mmd_154_l | 0.1243 | −0.1971 | 0.0389 | −0.2360 |
| HS_from_MQbrain_216_l | 0.1308 | −0.1971 | 0.0379 | −0.2350 |
| HSbrain_1377_l | 0.1390 | −0.0599 | 0.1746 | −0.2345 |
| HS_95_r | 0.0807 | −0.1187 | 0.1150 | −0.2338 |
| HS_from_MQbrain_631_l | 0.0913 | −0.0162 | 0.2174 | −0.2336 |
| Hsd_62_l | 0.9557 | 0.0230 | 0.2565 | −0.2334 |
| HSbrain_754_r | 0.0946 | −0.0715 | 0.1618 | −0.2334 |
| HS_from_MQbrain_467_l | 0.1975 | −0.0689 | 0.1635 | −0.2324 |
| hsa-let-7a-1_r | 0.0857 | −0.3519 | −0.1201 | −0.2319 |
| HS_from_MM_84_r | 0.0393 | 0.0468 | 0.2782 | −0.2314 |
| HS_from_PT_254_l | 0.0750 | −0.3623 | −0.1323 | −0.2300 |
| HS_from_MQbrain_1419_r | 0.1491 | 0.1676 | 0.3961 | −0.2285 |
| HS_from_MQbrain_1675_l | 0.1268 | −0.2295 | −0.0011 | −0.2284 |
| HS_from_MQbrain_117_r | 0.0772 | −0.2392 | −0.0108 | −0.2284 |
| hsa-mir-95_r | 0.1997 | −0.3852 | −0.1575 | −0.2277 |
| hsa-mir-204_l | 0.1975 | −0.5553 | −0.3280 | −0.2273 |
| hsa-mir-362_r | 0.0797 | −0.3608 | −0.1337 | −0.2271 |
| HS_from_MQbrain_604_l | 0.1597 | 0.3646 | 0.5916 | −0.2271 |
| hsa-mir-625_r | 0.1078 | 0.0307 | 0.2577 | −0.2270 |
| HS_from_MQbrain_1824_l | 0.1382 | 0.0296 | 0.2559 | −0.2263 |
| hsa-mir-329-1_r | 0.1243 | −0.2387 | −0.0133 | −0.2254 |
| HSbrain_114_r | 0.0928 | −0.1452 | 0.0796 | −0.2248 |
| HSbrain_627_r | 0.1482 | −0.4235 | −0.1994 | −0.2241 |
| hsa-mir-770_r | 0.1752 | 0.1895 | 0.4135 | −0.2240 |
| HS_from_MQbrain_1253_r | 0.0475 | −0.2381 | −0.0141 | −0.2240 |
| Hsd_from_Mmd_480_r | 0.1597 | 0.1689 | 0.3926 | −0.2237 |
| Hsd_from_Mmd_70_l | 0.1482 | 0.1279 | 0.3513 | −0.2234 |
| Hsd_83_l | 0.0961 | −0.1407 | 0.0815 | −0.2221 |
| HS_from_MQbrain_1855_l | 0.6805 | −0.1434 | 0.0785 | −0.2220 |
| HSbrain_594_l | 0.0913 | 0.4548 | 0.6763 | −0.2215 |
| HSbrain_113_r | 0.1703 | 0.0255 | 0.2461 | −0.2206 |
| HS_from_PT_60_l | 0.1196 | 0.1413 | 0.3612 | −0.2199 |
| hsa-mir-30b_l | 0.1196 | −0.1692 | 0.0494 | −0.2186 |
| HS_from_MQbrain_940_r | 0.1216 | 0.2172 | 0.4353 | −0.2181 |
| HS_from_MQbrain_1469_l | 0.0347 | 0.0551 | 0.2730 | −0.2179 |
| HS_74_r | 0.0961 | −0.1955 | 0.0222 | −0.2177 |
| hsa-mir-412_r | 0.0849 | 0.0196 | 0.2359 | −0.2163 |
| HS_from_MM_119_r | 0.1159 | −0.0200 | 0.1956 | −0.2156 |
| HS_189_r | 0.0582 | −0.1970 | 0.0183 | −0.2154 |
| HS_from_MQbrain_653_l | 0.1906 | −0.6406 | −0.4255 | −0.2150 |
| HSbrain_652_l | 0.1587 | 0.4008 | 0.6155 | −0.2147 |
| HSbrain_720_l | 0.1178 | −0.0996 | 0.1151 | −0.2147 |
| HS_86_r | 0.0995 | −0.1634 | 0.0513 | −0.2147 |
| HSbrain_1338_r | 0.1243 | −0.1516 | 0.0624 | −0.2140 |
| HSbrain_687_l | 0.1752 | 0.3318 | 0.5455 | −0.2138 |
| HS_from_MQbrain_1433_r | 0.1555 | 0.1663 | 0.3794 | −0.2131 |
| HS_from_MQbrain_36_r | 0.1887 | 0.4938 | 0.7067 | −0.2130 |
| hsa-mir-604_l | 0.0686 | −0.1664 | 0.0462 | −0.2126 |
| Hsd_from_Mmd_330_l | 0.0946 | 0.5116 | 0.7241 | −0.2125 |
| HS_from_MQbrain_56_l | 0.1146 | 0.4742 | 0.6867 | −0.2125 |
| HSbrain_158_l | 0.1597 | 0.0593 | 0.2717 | −0.2124 |
| Hsd_from_Mmd_527_l | 0.1752 | −0.1375 | 0.0745 | −0.2120 |
| HSbrain_744_l | 0.1774 | −0.3668 | −0.1557 | −0.2111 |
| Hsd_from_Mmd_23_l | 0.1196 | −0.2557 | −0.0446 | −0.2111 |
| HS_from_MQbrain_1294_l | 0.5817 | −0.2517 | −0.0407 | −0.2110 |
| HSbrain_184_r | 0.1078 | −0.1485 | 0.0626 | −0.2110 |
| hsa-mir-659_l | 0.0660 | −0.1505 | 0.0602 | −0.2107 |
| hsa-mir-155_r | 0.0686 | −0.1892 | 0.0210 | −0.2102 |
| HSbrain_673_l | 0.1587 | 0.4044 | 0.6144 | −0.2100 |
| Hsd_from_Mmd_93_r | 0.0913 | 0.1826 | 0.3925 | −0.2100 |
| HS_from_PT_27_r | 0.0824 | −0.0549 | 0.1546 | −0.2095 |
| HSbrain_728_l | 0.0946 | −0.1601 | 0.0485 | −0.2086 |
| HS_from_MQbrain_1001_r | 0.0750 | −0.2688 | −0.0603 | −0.2085 |
| HSbrain_365_l | 0.0895 | −0.0665 | 0.1420 | −0.2084 |
| HS_from_MQbrain_1684_r | 0.1308 | 0.0111 | 0.2188 | −0.2077 |
| HS_from_MQbrain_1340_l | 0.0583 | 0.1185 | 0.3262 | −0.2077 |
| HSbrain_661_l | 0.1026 | 0.0881 | 0.2952 | −0.2071 |
| HSbrain_102_r | 0.1482 | 0.4705 | 0.6774 | −0.2069 |
| HS_50_l | 0.1887 | 0.0232 | 0.2298 | −0.2065 |
| HS_from_PT_44_l | 0.1366 | −0.1863 | 0.0201 | −0.2064 |
| HSbrain_85_r | 0.1703 | −0.2468 | −0.0407 | −0.2062 |
| HS_from_MQbrain_921_l | 0.1078 | 0.1529 | 0.3587 | −0.2058 |
| Hsd_from_Mmd_223_r | 0.1646 | −0.5801 | −0.3745 | −0.2056 |
| HS_from_MM_92_r | 0.0152 | −0.1753 | 0.0302 | −0.2056 |
| HS_285_r | 0.1803 | 0.0291 | 0.2342 | −0.2051 |
| Hsd_from_Mmd_471_r | 0.1887 | −0.1205 | 0.0841 | −0.2046 |
| HS_from_MQbrain_1637_r | 0.1159 | 0.4653 | 0.6694 | −0.2041 |
| HSbrain_578_r | 0.1555 | −0.1478 | 0.0561 | −0.2039 |
| HS_from_MQbrain_288_r | 0.1646 | −0.0080 | 0.1957 | −0.2038 |
| HS_from_MQbrain_1292_r | 0.1159 | −0.1252 | 0.0783 | −0.2034 |
| HS_from_PT_299_r | 0.0723 | −0.0790 | 0.1233 | −0.2022 |
| HS_from_MQbrain_534_l | 0.0857 | −0.1529 | 0.0489 | −0.2018 |
| Hsd_82_r | 0.1216 | −0.1081 | 0.0918 | −0.1999 |
| HS_from_MM_198_r | 0.1943 | −0.0866 | 0.1132 | −0.1998 |
| HSbrain_677_l | 0.1107 | 0.3804 | 0.5796 | −0.1991 |
| HS_77_r | 0.1296 | 0.0324 | 0.2315 | −0.1991 |
| HSbrain_480_r | 0.1159 | 0.0211 | 0.2202 | −0.1991 |
| HS_67_r | 0.0838 | −0.2695 | −0.0706 | −0.1989 |
| hsa-mir-411_r | 0.0582 | −0.1454 | 0.0535 | −0.1989 |
| HS_from_MQbrain_789_r | 0.1975 | 0.1901 | 0.3883 | −0.1982 |
| hsa-mir-219-1_r | 0.1975 | −0.4213 | −0.2238 | −0.1975 |
| HSbrain_106_r | 0.1078 | 0.5758 | 0.7719 | −0.1961 |
| HS_from_MM_121_l | 0.0961 | −0.1851 | 0.0104 | −0.1954 |
| hsa-mir-643_r | 0.1735 | −0.2678 | −0.0729 | −0.1949 |
| hsa-mir-523_r | 0.0347 | −0.0856 | 0.1090 | −0.1946 |
| HS_from_MQbrain_1260_r | 0.0824 | −0.1165 | 0.0778 | −0.1943 |
| hsa-mir-135a-2_r | 0.1858 | 0.0702 | 0.2640 | −0.1938 |
| HS_303_r | 0.8378 | 0.3686 | 0.5622 | −0.1936 |
| HS_281_r | 0.1463 | −0.1699 | 0.0236 | −0.1935 |
| HS_from_MQbrain_969_l | 0.0750 | 0.0714 | 0.2648 | −0.1934 |
| HSbrain_431_r | 0.0772 | −0.2366 | −0.0434 | −0.1932 |
| HSbrain_705_l | 0.0895 | −0.0495 | 0.1436 | −0.1931 |
| HS_from_MQbrain_723_l | 0.0913 | −0.0666 | 0.1256 | −0.1922 |
| HS_142_l | 0.1858 | −0.0642 | 0.1274 | −0.1916 |
| HSbrain_359_l | 0.0913 | 0.0903 | 0.2810 | −0.1907 |
| HS_from_MM_1_l | 0.0723 | −0.1459 | 0.0441 | −0.1900 |
| HS_99_r | 0.1060 | −0.0002 | 0.1891 | −0.1893 |
| HSbrain_393_l | 0.1243 | −0.4464 | −0.2580 | −0.1883 |
| HSbrain_204_r | 0.0772 | −0.1078 | 0.0804 | −0.1881 |
| hsa-mir-301_l | 0.0646 | −0.1249 | 0.0628 | −0.1877 |
| HS_from_MQbrain_1292_l | 0.1482 | −0.4554 | −0.2683 | −0.1870 |
| Hsd_from_Mmd_405_l | 0.1045 | −0.2070 | −0.0201 | −0.1869 |
| HSbrain_408_r | 0.1616 | −0.0881 | 0.0988 | −0.1869 |
| HS_from_MQbrain_770_r | 0.1447 | −0.0536 | 0.1332 | −0.1868 |
| HS_from_MQbrain_429_l | 0.1463 | −0.2247 | −0.0393 | −0.1854 |
| HS_208_r | 0.1646 | 0.3023 | 0.4876 | −0.1854 |
| HS_from_MQbrain_133_r | 0.0268 | −0.0861 | 0.0992 | −0.1853 |
| HSbrain_621_r | 0.1146 | −0.2361 | −0.0510 | −0.1851 |
| HSbrain_225_r | 0.1196 | 0.1234 | 0.3079 | −0.1844 |
| HS_66_r | 0.1482 | −0.1679 | 0.0160 | −0.1839 |
| HSbrain_371_r | 0.0913 | −0.2014 | −0.0177 | −0.1837 |
| HS_from_MQbrain_1372_l | 0.1673 | −0.0771 | 0.1064 | −0.1835 |
| HS_from_MQbrain_108_r | 0.0686 | −0.1894 | −0.0065 | −0.1828 |
| HS_from_MQbrain_1088_l | 0.1491 | −0.2475 | −0.0649 | −0.1827 |
| HS_from_MQbrain_375_l | 0.1943 | 0.0136 | 0.1961 | −0.1825 |
| Hsd_from_Mmd_480_l | 0.1774 | 0.0106 | 0.1927 | −0.1821 |
| HS_91_l | 0.1382 | 0.0988 | 0.2809 | −0.1821 |
| HS_from_MQbrain_1657_r | 0.1390 | −0.1499 | 0.0316 | −0.1815 |
| HS_from_MQbrain_1808_r | 0.1308 | 0.3936 | 0.5746 | −0.1810 |
| hsa-mir-365-2_r | 0.1482 | −0.5563 | −0.3755 | −0.1808 |
| HSbrain_606_l | 0.1196 | 0.1888 | 0.3691 | −0.1803 |
| hsa-mir-453_l | 0.0327 | 0.0047 | 0.1848 | −0.1802 |
| HS_from_PT_117_r | 0.1230 | −0.0026 | 0.1772 | −0.1797 |
| HS_217_l | 0.1774 | 0.4782 | 0.6578 | −0.1796 |
| HS_from_MQbrain_1394_l | 0.1858 | −0.6321 | −0.4525 | −0.1796 |
| HSbrain_374_r | 0.0583 | −0.1743 | 0.0053 | −0.1796 |
| HS_from_MQbrain_635_r | 0.1975 | −0.6176 | −0.4391 | −0.1784 |
| HSbrain_717_r | 0.0913 | −0.2831 | −0.1057 | −0.1774 |
| Hsd_37_r | 0.1045 | −0.1041 | 0.0732 | −0.1773 |
| Hsd_from_Mmd_347_l | 0.1597 | −0.0685 | 0.1087 | −0.1771 |
| hsa-mir-191_r | 0.1522 | −0.2760 | −0.0991 | −0.1769 |
| HS_from_MQbrain_1649_r | 0.0995 | −0.2685 | −0.0919 | −0.1766 |
| HS_from_MQbrain_1272_l | 0.9974 | −0.0850 | 0.0916 | −0.1766 |
| HSbrain_123_l | 0.0961 | 0.0016 | 0.1781 | −0.1765 |
| HSbrain_689_r | 0.1803 | −0.2996 | −0.1232 | −0.1764 |
| HS_from_PT_139_r | 0.1159 | 0.0516 | 0.2272 | −0.1756 |
| HSbrain_16_l | 0.4115 | −0.1118 | 0.0634 | −0.1752 |
| HSbrain_1413_l | 0.1131 | 0.1125 | 0.2869 | −0.1745 |
| HS_from_MQbrain_62_l | 0.1045 | −0.2962 | −0.1218 | −0.1744 |
| HS_from_MQbrain_1896_r | 0.0772 | −0.2758 | −0.1017 | −0.1741 |
| HS_from_MQbrain_93_r | 0.0715 | −0.1611 | 0.0130 | −0.1740 |
| HS_105_l | 0.1906 | 0.0160 | 0.1900 | −0.1740 |
| HS_from_MQbrain_283_r | 0.1463 | −0.2255 | −0.0518 | −0.1738 |
| HSbrain_580_r | 0.0750 | −0.1445 | 0.0292 | −0.1737 |
| HS_from_MQbrain_914_l | 0.1447 | −0.1587 | 0.0150 | −0.1737 |
| HS_from_MQbrain_1545_r | 0.1060 | −0.4218 | −0.2482 | −0.1736 |
| Hsd_from_Mmd_410_r | 0.0764 | −0.1101 | 0.0625 | −0.1725 |
| HS_from_MQbrain_268_r | 0.1243 | −0.0744 | 0.0978 | −0.1722 |
| HS_76_r | 0.1178 | −0.1154 | 0.0566 | −0.1720 |
| HS_from_MQbrain_1593_r | 0.0883 | −0.1606 | 0.0103 | −0.1709 |
| HS_from_MQbrain_681_l | 0.1243 | 0.0405 | 0.2111 | −0.1707 |
| hsa-mir-133a-1_r | 0.0686 | −0.0303 | 0.1403 | −0.1706 |
| Hsd_from_Mmd_138_l | 0.1026 | −0.1817 | −0.0111 | −0.1705 |
| HSbrain_509_l | 0.1390 | −0.1567 | 0.0138 | −0.1705 |
| HS_from_MQbrain_229_l | 0.1997 | −0.3770 | −0.2069 | −0.1701 |
| HSbrain_1337_l | 0.1975 | −0.0408 | 0.1291 | −0.1698 |
| HS_from_MQbrain_1773_l | 0.1491 | 0.4936 | 0.6634 | −0.1698 |
| HSbrain_62_r | 0.0723 | 0.0515 | 0.2213 | −0.1698 |
| Hsd_from_Mmd_215_r | 0.0750 | −0.1552 | 0.0144 | −0.1697 |
| hsa-mir-588_r | 0.1616 | −0.2072 | −0.0385 | −0.1687 |
| HSbrain_643_l | 0.1752 | 0.0096 | 0.1777 | −0.1680 |
| Hsd_from_Mmd_217_r | 0.1803 | −0.1955 | −0.0280 | −0.1675 |
| HS_from_MQbrain_1031_r | 0.0995 | −0.3459 | −0.1786 | −0.1673 |
| HSbrain_468_r | 0.0715 | 0.0630 | 0.2298 | −0.1669 |
| HS_from_MQbrain_1556_r | 0.1752 | −0.3436 | −0.1771 | −0.1665 |
| HS_from_MQbrain_1708_l | 0.1752 | 0.0083 | 0.1743 | −0.1661 |
| HS_from_MQbrain_212_r | 0.1997 | −0.1164 | 0.0494 | −0.1658 |
| Hsd_from_Mmd_81_r | 0.1943 | −0.2170 | −0.0521 | −0.1649 |
| HS_from_MQbrain_691_r | 0.1887 | −0.3130 | −0.1483 | −0.1647 |
| HS_from_PT_27_l | 0.1906 | −0.1018 | 0.0621 | −0.1639 |
| HS_190_r | 0.1463 | 0.0420 | 0.2057 | −0.1637 |
| HS_from_MQbrain_566_l | 0.0928 | 0.0128 | 0.1755 | −0.1627 |
| HSbrain_461_r | 0.1146 | −0.1663 | −0.0037 | −0.1626 |
| HS_47_l | 0.1803 | −0.2518 | −0.0898 | −0.1620 |
| HS_from_MQbrain_332_l | 0.1414 | −0.2211 | −0.0595 | −0.1616 |
| HS_from_MQbrain_266_r | 0.0750 | −0.0878 | 0.0737 | −0.1615 |
| HS_61_r | 0.1703 | −0.5331 | −0.3716 | −0.1615 |
| HS_from_MQbrain_710_r | 0.1703 | 0.1180 | 0.2786 | −0.1606 |
| hsa-mir-616_r | 0.1555 | −0.1860 | −0.0254 | −0.1606 |
| HS_from_MQbrain_482_l | 0.1703 | −0.1412 | 0.0188 | −0.1600 |
| HS_from_MM_80_l | 0.1906 | −0.1170 | 0.0431 | −0.1600 |
| hsa-mir-30b_r | 0.1454 | 0.1418 | 0.3007 | −0.1589 |
| Hsd_from_Mmd_424_l | 0.1555 | −0.1584 | 0.0002 | −0.1586 |
| HS_292_l | 0.4115 | −0.0648 | 0.0934 | −0.1582 |
| Hsd_from_Mmd_396_l | 0.1390 | −0.3295 | −0.1716 | −0.1579 |
| HS_from_MQbrain_1722_l | 0.1616 | 0.2108 | 0.3684 | −0.1576 |
| HSbrain_591_l | 0.0946 | 0.2730 | 0.4305 | −0.1575 |
| HSbrain_794_l | 0.1774 | 0.2067 | 0.3638 | −0.1571 |
| HS_from_MM_311_l | 0.1673 | −0.2655 | −0.1088 | −0.1567 |
| Hsd_12_r | 0.1131 | −0.2890 | −0.1328 | −0.1562 |
| HS_from_MQbrain_1443_r | 0.0849 | 0.3428 | 0.4988 | −0.1560 |
| HSbrain_506_r | 0.1587 | −0.1682 | −0.0125 | −0.1557 |
| HSbrain_472_l | 0.0913 | −0.0300 | 0.1255 | −0.1555 |
| HSbrain_50_l | 0.1131 | 0.1314 | 0.2857 | −0.1543 |
| HS_from_PT_115_r | 0.1752 | −0.1110 | 0.0432 | −0.1542 |
| HSbrain_696_r | 0.1078 | −0.0376 | 0.1164 | −0.1540 |
| HSbrain_495_r | 0.1482 | 0.1239 | 0.2778 | −0.1539 |
| HS_50_r | 0.1308 | −0.1876 | −0.0341 | −0.1535 |
| HSbrain_809_r | 0.1906 | 0.2931 | 0.4465 | −0.1534 |
| HS_from_MQbrain_1549_l | 0.1347 | −0.2547 | −0.1022 | −0.1525 |
| HS_77_l | 0.1858 | 0.0084 | 0.1598 | −0.1514 |
| HS_from_MQbrain_699_r | 0.1673 | 0.1013 | 0.2519 | −0.1506 |
| HSbrain_111_l | 0.5053 | −0.1097 | 0.0400 | −0.1497 |
| hsa-mir-365-2_l | 0.0750 | 0.0933 | 0.2418 | −0.1484 |
| hsa-mir-655_l | 0.1463 | −0.0490 | 0.0991 | −0.1481 |
| HSbrain_214_l | 0.4679 | 0.0466 | 0.1946 | −0.1480 |
| HS_138_l | 0.1843 | −0.1893 | −0.0420 | −0.1474 |
| HSbrain_139_r | 0.1454 | −0.1193 | 0.0278 | −0.1471 |
| Hsd_95_l | 0.1906 | −0.6306 | −0.4846 | −0.1460 |
| HS_from_MQbrain_397_l | 0.1906 | −0.1741 | −0.0283 | −0.1458 |
| HS_from_MQbrain_444_r | 0.1646 | 0.1288 | 0.2734 | −0.1446 |
| HS_from_MM_190_l | 0.1975 | −0.4987 | −0.3542 | −0.1445 |
| HSbrain_690_l | 0.1774 | 0.0326 | 0.1771 | −0.1445 |
| hsa-mir-146a_r | 0.0849 | −0.0432 | 0.1010 | −0.1442 |
| hsa-mir-550-2_r | 0.1943 | −0.0900 | 0.0541 | −0.1441 |
| hsa-mir-296_l | 0.1646 | −0.0608 | 0.0819 | −0.1427 |
| HS_182_r | 0.1843 | −0.2247 | −0.0828 | −0.1419 |
| Hsd_from_Mmd_176_r | 0.1703 | −0.0565 | 0.0852 | −0.1417 |
| HS_109_r | 0.1774 | −0.0011 | 0.1404 | −0.1415 |
| HSbrain_651_r | 0.1997 | 0.2192 | 0.3603 | −0.1411 |
| HS_from_MQbrain_1517_r | 0.1997 | 0.1558 | 0.2969 | −0.1411 |
| HSbrain_231_l | 0.0772 | 0.1866 | 0.3274 | −0.1407 |
| Hsd_from_Mmd_511_r | 0.0883 | −0.2432 | −0.1055 | −0.1378 |
| Hsd_from_Mmd_530_l | 0.1943 | −0.2262 | −0.0894 | −0.1368 |
| HS_from_MQbrain_1365_l | 0.1906 | 0.0966 | 0.2333 | −0.1366 |
| HS_from_MQbrain_886_r | 0.1390 | 0.0272 | 0.1632 | −0.1360 |
| HSbrain_771_l | 0.1268 | 0.2639 | 0.3996 | −0.1357 |
| HS_from_MQbrain_272_l | 0.1858 | 0.0712 | 0.2065 | −0.1353 |
| hsa-mir-217_r | 0.1463 | −0.2236 | −0.0892 | −0.1344 |
| HS_from_MQbrain_1776_r | 0.1216 | 0.0480 | 0.1819 | −0.1338 |
| HS_54_r | 0.0849 | −0.0037 | 0.1294 | −0.1332 |
| HSbrain_588_r | 0.1997 | 0.1444 | 0.2767 | −0.1324 |
| Hsd_from_Mmd_501_l | 0.1482 | −0.4092 | −0.2770 | −0.1321 |
| HSbrain_220_r | 0.1347 | 0.0358 | 0.1672 | −0.1314 |
| hsa-mir-374_l | 0.1774 | −0.3352 | −0.2048 | −0.1304 |
| Hsd_from_Mmd_509_l | 0.9380 | −0.1612 | −0.0309 | −0.1303 |
| HS_from_MQbrain_1215_l | 0.1616 | −0.0358 | 0.0941 | −0.1300 |
| HS_from_MQbrain_968_r | 0.1906 | 0.2716 | 0.4008 | −0.1292 |
| HSbrain_75_l | 0.1463 | 0.3039 | 0.4328 | −0.1289 |
| HSbrain_593_r | 0.1803 | 0.0733 | 0.2020 | −0.1286 |
| HS_from_MQbrain_1677_r | 0.1026 | −0.1597 | −0.0327 | −0.1271 |
| hsa-mir-579_r | 0.1555 | −0.2613 | −0.1347 | −0.1266 |
| HS_from_MQbrain_496_r | 0.1703 | 0.1682 | 0.2947 | −0.1265 |
| hsa-mir-365-1_l | 0.1803 | 0.0808 | 0.2059 | −0.1251 |
| Hsd_from_Mmd_500_l | 0.1463 | −0.1701 | −0.0480 | −0.1221 |
| Hsd_from_Mmd_97_l | 0.1735 | −0.1030 | 0.0191 | −0.1221 |
| HS_from_MM_86_l | 0.8260 | −0.2663 | −0.1444 | −0.1219 |
| HS_from_MQbrain_1667_r | 0.1703 | −0.1889 | −0.0693 | −0.1197 |
| HSbrain_970_l | 0.1752 | 0.0116 | 0.1296 | −0.1180 |
| HS_from_MQbrain_55_l | 0.1943 | −0.0142 | 0.1033 | −0.1175 |
| HS_from_MM_18_r | 0.0772 | −0.0044 | 0.1124 | −0.1168 |
| HS_from_MQbrain_266_l | 0.1230 | −0.1340 | −0.0176 | −0.1164 |
| HS_from_MQbrain_626_r | 0.1906 | −0.2955 | −0.1793 | −0.1162 |
| HS_from_MQbrain_188_l | 0.1843 | −0.1215 | −0.0061 | −0.1154 |
| hsa-mir-510_r | 0.1447 | −0.1753 | −0.0616 | −0.1137 |
| HS_from_MQbrain_22_l | 0.1858 | −0.1168 | −0.0031 | −0.1137 |
| HS_from_MQbrain_405_r | 0.1906 | 0.0400 | 0.1535 | −0.1135 |
| hsa-mir-132_l | 0.1673 | −0.1141 | −0.0028 | −0.1113 |
| HS_from_MQbrain_536_r | 0.1555 | 0.0700 | 0.1803 | −0.1103 |
| HS_from_MQbrain_686_l | 0.1587 | −0.1259 | −0.0163 | −0.1096 |
| hsa-mir-488_l | 0.1414 | −0.0494 | 0.0599 | −0.1094 |
| HS_278_l | 0.1382 | 0.1250 | 0.2333 | −0.1083 |
| HSbrain_590_l | 0.1347 | −0.0842 | 0.0232 | −0.1075 |
| HS_from_MQbrain_1164_l | 0.1454 | 0.0403 | 0.1464 | −0.1061 |
| HSbrain_621_l | 0.1843 | −0.0817 | 0.0237 | −0.1054 |
| HS_from_MM_200_l | 0.1326 | −0.1041 | 0.0013 | −0.1054 |
| HSbrain_583_r | 0.0583 | −0.1173 | −0.0120 | −0.1053 |
| HS_from_MQbrain_1412_l | 0.1230 | 0.1293 | 0.2345 | −0.1053 |
| HSbrain_337_r | 0.1390 | −0.0716 | 0.0318 | −0.1034 |
| hsa-mir-568_l | 0.1997 | −0.1221 | −0.0196 | −0.1025 |
| hsa-mir-518e_r | 0.1887 | −0.1142 | −0.0117 | −0.1025 |
| HS_from_MQbrain_1346_r | 0.1887 | −0.2318 | −0.1299 | −0.1019 |
| HSbrain_540_l | 0.1752 | 0.0056 | 0.1047 | −0.0991 |
| HS_from_MQbrain_1261_r | 0.1230 | −0.0828 | 0.0160 | −0.0988 |
| hsa-mir-124a-1_l | 0.0723 | 0.0336 | 0.1293 | −0.0957 |
| HS_from_MQbrain_1470_l | 0.1178 | −0.0139 | 0.0777 | −0.0916 |
| HS_from_MQbrain_722_l | 0.1308 | −0.0176 | 0.0720 | −0.0896 |
| Hsd_from_Mmd_443_l | 0.1673 | −0.0381 | 0.0485 | −0.0866 |
| HS_from_MQbrain_1224_r | 0.1482 | −0.0994 | −0.0129 | −0.0865 |
| Hsd_from_Mmd_181_r | 0.1159 | −0.0374 | 0.0490 | −0.0864 |
| HS_223_r | 0.1414 | 0.0531 | 0.1369 | −0.0838 |
| Hsd_from_Mmd_503_l | 0.1308 | −0.1190 | −0.0353 | −0.0836 |
| HS_from_MM_288_l | 0.1454 | −0.0096 | 0.0740 | −0.0836 |
| hsa-mir-646_r | 0.0995 | −0.0048 | 0.0781 | −0.0829 |
| hsa-mir-638_r | 0.0583 | 0.0812 | 0.1608 | −0.0796 |
| HS_56_l | 0.1308 | −0.0116 | 0.0680 | −0.0796 |
| HS_from_MQbrain_221_r | 0.1026 | −0.0042 | 0.0746 | −0.0788 |
| Hsd_from_Mmd_253_l | 0.1906 | −0.0633 | 0.0133 | −0.0767 |
| HS_from_MQbrain_1347_r | 0.1906 | −0.0721 | 0.0038 | −0.0759 |
| hsa-mir-454_l | 0.1366 | −0.0405 | 0.0339 | −0.0744 |
| HS_from_MM_102_r | 0.1347 | −0.0463 | 0.0268 | −0.0731 |
| Hsd_from_Mmd_178_l | 0.1975 | −0.1519 | −0.0804 | −0.0715 |
| HS_from_MQbrain_1301_r | 0.0727 | 0.0246 | 0.0959 | −0.0712 |
| HS_from_PT_196_l | 0.0807 | −0.0244 | 0.0458 | −0.0702 |
| HS_88_r | 0.1296 | 0.0150 | 0.0851 | −0.0702 |
| Hsd_30_l | 0.0961 | 0.0292 | 0.0977 | −0.0685 |
| HSbrain_792_r | 0.0807 | 0.0264 | 0.0947 | −0.0683 |
| HS_133_l | 0.1463 | −0.0065 | 0.0603 | −0.0668 |
| HS_from_MM_48_l | 0.1366 | 0.1246 | 0.1911 | −0.0665 |
| HS_from_MM_150_r | 0.0995 | −0.0279 | 0.0375 | −0.0654 |
| HSbrain_698_r | 0.1414 | −0.0004 | 0.0647 | −0.0651 |
| HS_from_MQbrain_355_l | 0.0807 | −0.0045 | 0.0599 | −0.0644 |
| hsa-mir-623_r | 0.1943 | −0.0515 | 0.0121 | −0.0636 |
| HS_from_MQbrain_897_r | 0.1268 | −0.0204 | 0.0421 | −0.0626 |
| hsa-mir-369_l | 0.1382 | −0.0286 | 0.0338 | −0.0624 |
| Hsd_8_r | 0.1858 | −0.0038 | 0.0586 | −0.0623 |
| HS_237_r | 0.1366 | 0.0169 | 0.0784 | −0.0616 |
| hsa-mir-16-1_r | 0.1230 | −0.0390 | 0.0196 | −0.0586 |
| HS_from_MQbrain_161_l | 0.1382 | 0.0008 | 0.0592 | −0.0584 |
| HS_from_MQbrain_2087_r | 0.1107 | 0.0153 | 0.0700 | −0.0547 |
| HS_from_MQbrain_348_r | 0.1366 | −0.0149 | 0.0393 | −0.0543 |
| HSbrain_244_l | 0.1482 | 0.0418 | 0.0947 | −0.0529 |
| HS_286_r | 0.1482 | −0.0309 | 0.0193 | −0.0502 |
| HSbrain_791_l | 0.4298 | 0.0097 | 0.0585 | −0.0488 |
| HS_from_MQbrain_716_l | 0.1026 | 0.0412 | 0.0882 | −0.0470 |
| HSbrain_692_l | 0.1597 | 0.0513 | 0.0981 | −0.0468 |
| HS_from_MQbrain_661_r | 0.1803 | 0.0243 | 0.0554 | −0.0310 |
| HSbrain_541_r | 0.1230 | 0.0318 | −0.0137 | 0.0456 |
| HS_45_r | 0.1522 | 0.0495 | −0.0050 | 0.0546 |
| HS_from_MQbrain_1569_l | 0.2259 | 0.0655 | 0.0071 | 0.0584 |
| HSbrain_599_r | 0.1616 | 0.0538 | −0.0110 | 0.0648 |
| HS_from_MQbrain_181_l | 0.0983 | 0.1268 | 0.0570 | 0.0698 |
| HS_from_MQbrain_1263_r | 0.0552 | 0.0127 | −0.0594 | 0.0720 |
| HSbrain_222_r | 0.1366 | 0.1026 | 0.0247 | 0.0779 |
| HS_from_MQbrain_859_l | 0.1887 | 0.2026 | 0.1234 | 0.0791 |
| HS_from_MM_124_l | 0.1616 | −0.0937 | −0.1730 | 0.0794 |
| HS_from_MQbrain_660_l | 0.0518 | 0.0998 | 0.0085 | 0.0913 |
| HSbrain_166_l | 0.1858 | −0.0447 | −0.1380 | 0.0932 |
| Hsd_from_Mmd_398_r | 0.1414 | −0.0587 | −0.1524 | 0.0937 |
| HS_113_l | 0.1587 | 0.0450 | −0.0512 | 0.0962 |
| HS_from_MQbrain_1100_r | 0.1997 | 0.0682 | −0.0296 | 0.0979 |
| HSbrain_80_l | 0.1858 | −0.0297 | −0.1284 | 0.0987 |
| HS_from_MQbrain_1110_l | 0.1597 | 0.0443 | −0.0555 | 0.0998 |
| HS_from_MQbrain_1127_r | 0.1060 | 0.2349 | 0.1325 | 0.1024 |
| HS_from_MQbrain_38_r | 0.1326 | 0.1501 | 0.0452 | 0.1048 |
| HSbrain_545_r | 0.0142 | 0.0498 | −0.0564 | 0.1061 |
| HSbrain_757_r | 0.0824 | −0.0055 | −0.1133 | 0.1078 |
| HS_from_PT_211_r | 0.1774 | −0.0027 | −0.1119 | 0.1092 |
| HS_from_MQbrain_1091_l | 0.1752 | 0.0075 | −0.1034 | 0.1110 |
| HS_from_MQbrain_1354_r | 0.1858 | −0.0965 | −0.2127 | 0.1162 |
| HS_from_PT_91_r | 0.1597 | 0.0486 | −0.0679 | 0.1164 |
| HS_from_MQbrain_1421_l | 0.1943 | 0.1286 | 0.0121 | 0.1165 |
| Hsd_from_Mmd_197_l | 0.1587 | 0.0412 | −0.0754 | 0.1166 |
| HS_130_l | 0.9053 | 0.1136 | −0.0044 | 0.1180 |
| HSbrain_266_r | 0.1045 | 0.0253 | −0.0941 | 0.1194 |
| HS_from_MQbrain_1845_l | 0.1326 | −0.0250 | −0.1500 | 0.1250 |
| HS_from_MQbrain_734_l | 0.1752 | −0.1549 | −0.2813 | 0.1264 |
| HS_from_MQbrain_1777_l | 0.1752 | −0.2554 | −0.3845 | 0.1292 |
| HS_from_MQbrain_1747_l | 0.1997 | 0.1017 | −0.0294 | 0.1310 |
| HSbrain_177_r | 0.1491 | −0.1879 | −0.3196 | 0.1316 |
| HS_from_MQbrain_1632_l | 0.1216 | 0.0119 | −0.1227 | 0.1346 |
| Hsd_32_r | 0.1390 | 0.0226 | −0.1123 | 0.1350 |
| HSbrain_803_l | 0.0575 | −0.0430 | −0.1804 | 0.1374 |
| Hsd_from_Mmd_189_l | 0.0764 | −0.1958 | −0.3349 | 0.1391 |
| HS_from_MQbrain_1405_l | 0.1997 | 0.5801 | 0.4407 | 0.1394 |
| HSbrain_663_l | 0.1616 | 0.1353 | −0.0050 | 0.1403 |
| HS_from_MQbrain_1876_r | 0.1673 | 0.1051 | −0.0379 | 0.1431 |
| HS_from_PT_134_l | 0.1491 | −0.2217 | −0.3658 | 0.1441 |
| HS_265_l | 0.1597 | 0.1908 | 0.0446 | 0.1463 |
| hsa-mir-338_l | 0.1555 | −0.1400 | −0.2904 | 0.1503 |
| HS_from_MQbrain_715_l | 0.1326 | −0.0648 | −0.2180 | 0.1532 |
| HS_from_MQbrain_550_l | 0.1803 | −0.1402 | −0.2938 | 0.1536 |
| HSbrain_270_r | 0.1887 | 0.4395 | 0.2855 | 0.1540 |
| HS_from_MQbrain_1070_r | 0.1326 | −0.1357 | −0.2898 | 0.1541 |
| HSbrain_32_l | 0.1858 | −0.1929 | −0.3496 | 0.1568 |
| HS_from_MQbrain_1134_r | 0.0686 | −0.0065 | −0.1636 | 0.1571 |
| HSbrain_316_l | 0.3713 | −0.1454 | −0.3027 | 0.1573 |
| HS_83_r | 0.0857 | 0.0508 | −0.1071 | 0.1580 |
| hsa-mir-204_r | 0.1447 | −0.2259 | −0.3840 | 0.1581 |
| HS_from_MQbrain_57_l | 0.1735 | 0.0953 | −0.0629 | 0.1582 |
| HSbrain_305_r | 0.1216 | −0.1436 | −0.3029 | 0.1593 |
| HSbrain_347_l | 0.1774 | −0.2324 | −0.3918 | 0.1594 |
| Hsd_24_r | 0.1975 | −0.0114 | −0.1712 | 0.1598 |
| HS_from_MQbrain_991_r | 0.1803 | 0.1429 | −0.0174 | 0.1603 |
| HS_from_MM_199_l | 0.1463 | 0.3322 | 0.1697 | 0.1625 |
| HSbrain_307_r | 0.1463 | −0.1357 | −0.2985 | 0.1628 |
| HS_from_MQbrain_70_l | 0.1382 | −0.1727 | −0.3365 | 0.1638 |
| HS_from_MQbrain_675_l | 0.1906 | −0.4637 | −0.6279 | 0.1641 |
| Hsd_from_Mmd_155_l | 0.1326 | −0.1095 | −0.2741 | 0.1646 |
| HS_from_MQbrain_58_l | 0.0715 | 0.1904 | 0.0244 | 0.1660 |
| HS_from_MQbrain_435_r | 0.0727 | 0.0635 | −0.1030 | 0.1665 |
| hsa-mir-224_r | 0.0961 | 0.0285 | −0.1386 | 0.1671 |
| HS_from_MQbrain_1331_r | 0.1414 | −0.1794 | −0.3465 | 0.1671 |
| HS_from_MQbrain_1793_r | 0.0961 | −0.1079 | −0.2750 | 0.1671 |
| HS_from_MQbrain_500_r | 0.1975 | 0.3684 | 0.2013 | 0.1671 |
| hsa-mir-622_l | 0.1597 | −0.1693 | −0.3370 | 0.1676 |
| hsa-mir-196a-2_r | 0.1382 | 0.0451 | −0.1226 | 0.1677 |
| HSbrain_799_r | 0.0824 | 0.2293 | 0.0603 | 0.1690 |
| HSbrain_588_l | 0.1131 | 0.0224 | −0.1503 | 0.1727 |
| HSbrain_622_r | 0.1491 | 0.1098 | −0.0630 | 0.1728 |
| HS_from_MQbrain_382_l | 0.1326 | 0.0976 | −0.0752 | 0.1728 |
| Hsd_from_Mmd_371_r | 0.0582 | −0.0728 | −0.2467 | 0.1739 |
| HSbrain_440_l | 0.1131 | −0.0596 | −0.2359 | 0.1763 |
| HS_from_MQbrain_600_l | 0.1366 | −0.0334 | −0.2099 | 0.1765 |
| HS_from_PT_140_l | 0.1010 | 0.2516 | 0.0718 | 0.1799 |
| HS_from_PT_238_l | 0.1906 | −0.4074 | −0.5879 | 0.1805 |
| HSbrain_717_l | 0.1522 | −0.2717 | −0.4527 | 0.1810 |
| HS_167_r | 0.1597 | 0.2377 | 0.0559 | 0.1818 |
| HS_from_PT_34_r | 0.1752 | −0.1093 | −0.2912 | 0.1819 |
| HS_from_MQbrain_1355_r | 0.0895 | 0.1201 | −0.0637 | 0.1838 |
| HSbrain_779_r | 0.1390 | 0.1375 | −0.0471 | 0.1846 |
| HSbrain_753_r | 0.1382 | −0.0459 | −0.2316 | 0.1857 |
| Hsd_from_Mmd_237_l | 0.0715 | 0.0317 | −0.1542 | 0.1859 |
| HS_from_MQbrain_974_l | 0.1196 | −0.4263 | −0.6131 | 0.1868 |
| HSbrain_235_l | 0.0995 | 0.0495 | −0.1380 | 0.1875 |
| HS_from_MQbrain_1143_l | 0.1447 | 0.4623 | 0.2739 | 0.1884 |
| HS_from_MQbrain_1836_l | 0.1382 | 0.0869 | −0.1029 | 0.1899 |
| HS_208_l | 0.0857 | 0.1582 | −0.0327 | 0.1910 |
| Hsd_from_Mmd_535_r | 0.1463 | −0.2617 | −0.4530 | 0.1912 |
| HS_from_MQbrain_1631_r | 0.1803 | −0.2025 | −0.3937 | 0.1913 |
| HSbrain_402_l | 0.1230 | 0.0139 | −0.1784 | 0.1923 |
| HSbrain_483_r | 0.1735 | 0.3383 | 0.1454 | 0.1928 |
| HS_from_MQbrain_1204_r | 0.1454 | −0.2815 | −0.4745 | 0.1930 |
| Hsd_84_r | 0.1843 | −0.0011 | −0.1954 | 0.1944 |
| HSbrain_755_l | 0.1943 | −0.2320 | −0.4270 | 0.1950 |
| HS_from_MQbrain_842_l | 0.1146 | 0.2444 | 0.0494 | 0.1950 |
| HS_from_PT_126_l | 0.1887 | 0.1428 | −0.0524 | 0.1953 |
| Hsd_85_r | 0.0347 | 0.0470 | −0.1495 | 0.1964 |
| HS_from_MQbrain_81_l | 0.0913 | −0.1585 | −0.3556 | 0.1971 |
| HS_from_MQbrain_401_l | 0.1673 | −0.1565 | −0.3546 | 0.1981 |
| HS_from_MQbrain_1752_l | 0.1752 | 0.1844 | −0.0138 | 0.1982 |
| HS_from_MM_103_l | 0.0750 | 0.0450 | −0.1537 | 0.1987 |
| HS_from_MQbrain_1420_l | 0.1347 | −0.1887 | −0.3875 | 0.1989 |
| HS_111_r | 0.1522 | −0.2558 | −0.4548 | 0.1990 |
| HSbrain_627_l | 0.0838 | −0.0780 | −0.2773 | 0.1993 |
| HSbrain_1414_r | 0.1774 | −0.2852 | −0.4850 | 0.1998 |
| HS_101_l | 0.1326 | −0.2799 | −0.4797 | 0.1998 |
| HSbrain_289_r | 0.1975 | 0.4494 | 0.2486 | 0.2008 |
| HSbrain_236_l | 0.0807 | 0.0995 | −0.1022 | 0.2017 |
| Hsd_106_l | 0.0849 | −0.1324 | −0.3348 | 0.2025 |
| HS_from_MQbrain_628_l | 0.8957 | 0.0715 | −0.1310 | 0.2025 |
| hsa-mir-148a_l | 0.0528 | 0.0097 | −0.1991 | 0.2088 |
| HS_from_MM_251_r | 0.0393 | −0.0801 | −0.2890 | 0.2089 |
| HS_220_l | 0.1587 | 0.0739 | −0.1351 | 0.2090 |
| HSbrain_568_r | 0.1774 | 0.0532 | −0.1559 | 0.2091 |
| HSbrain_783_l | 0.1308 | −0.0263 | −0.2354 | 0.2091 |
| HS_from_MQbrain_1748_r | 0.1010 | 0.0386 | −0.1705 | 0.2092 |
| HS_from_MM_131_l | 0.1843 | −0.0517 | −0.2614 | 0.2097 |
| HS_from_MQbrain_819_r | 0.1906 | −0.3540 | −0.5641 | 0.2101 |
| HS_from_MQbrain_1075_l | 0.1230 | −0.4380 | −0.6483 | 0.2102 |
| HS_119_l | 0.0772 | 0.1335 | −0.0767 | 0.2102 |
| HS_from_MQbrain_1494_r | 0.0849 | −0.0801 | −0.2906 | 0.2105 |
| HS_from_MQbrain_1832_l | 0.0715 | −0.1609 | −0.3718 | 0.2109 |
| Hsd_from_Mmd_261_l | 0.0849 | −0.0120 | −0.2232 | 0.2112 |
| HS_39_r | 0.1414 | −0.2181 | −0.4298 | 0.2117 |
| HS_from_MQbrain_1645_l | 0.1646 | −0.0691 | −0.2820 | 0.2130 |
| HS_62_l | 0.1347 | −0.3506 | −0.5637 | 0.2131 |
| HSbrain_187_r | 0.0501 | 0.0264 | −0.1868 | 0.2133 |
| HSbrain_787_l | 0.1454 | 0.4111 | 0.1961 | 0.2150 |
| HS_from_MQbrain_1470_r | 0.1774 | 0.4504 | 0.2350 | 0.2154 |
| HS_from_MQbrain_869_l | 0.0727 | −0.1902 | −0.4057 | 0.2155 |
| HSbrain_780_r | 0.0501 | 0.1250 | −0.0914 | 0.2164 |
| HSbrain_755_r | 0.1975 | −0.4185 | −0.6350 | 0.2165 |
| HS_58_l | 0.1491 | 0.2550 | 0.0375 | 0.2175 |
| HSbrain_328_l | 0.0824 | 0.0093 | −0.2094 | 0.2187 |
| HS_54_l | 0.1454 | −0.0577 | −0.2771 | 0.2194 |
| HSbrain_644_r | 0.1597 | 0.3969 | 0.1772 | 0.2197 |
| HS_from_MQbrain_72_r | 0.0913 | −0.0812 | −0.3023 | 0.2212 |
| HS_102_l | 0.1146 | −0.3072 | −0.5284 | 0.2212 |
| HSbrain_692_r | 0.1774 | 0.1442 | −0.0771 | 0.2213 |
| HS_from_MQbrain_1164_r | 0.1146 | 0.0905 | −0.1309 | 0.2214 |
| HSbrain_25_r | 0.0624 | 0.0766 | −0.1466 | 0.2232 |
| HSbrain_336_l | 0.6371 | 0.3315 | 0.1075 | 0.2240 |
| HSbrain_708_l | 0.1196 | −0.2608 | −0.4850 | 0.2242 |
| Hsd_from_Mmd_487_r | 0.1414 | −0.3042 | −0.5289 | 0.2248 |
| HS_49_r | 0.0883 | 0.3921 | 0.1671 | 0.2250 |
| HS_from_MQbrain_22_r | 0.1060 | −0.0274 | −0.2525 | 0.2251 |
| Hsd_from_Mmd_52_r | 0.1107 | −0.4921 | −0.7174 | 0.2253 |
| HSbrain_479_l | 0.1975 | 0.3579 | 0.1326 | 0.2253 |
| HS_from_PT_188_l | 0.1491 | −0.0426 | −0.2680 | 0.2255 |
| HS_110_r | 0.1491 | −0.4924 | −0.7179 | 0.2255 |
| HS_from_MQbrain_941_r | 0.0169 | 0.0176 | −0.2083 | 0.2259 |
| HSbrain_210_l | 0.1975 | −0.1228 | −0.3499 | 0.2270 |
| HSbrain_452_l | 0.0983 | 0.0356 | −0.1920 | 0.2276 |
| HSbrain_647_r | 0.1482 | −0.0282 | −0.2564 | 0.2282 |
| HSbrain_431_l | 0.1587 | −0.3079 | −0.5368 | 0.2290 |
| HSbrain_82_r | 0.1447 | 0.2629 | 0.0330 | 0.2300 |
| HSbrain_182_r | 0.0723 | −0.1808 | −0.4110 | 0.2302 |
| HSbrain_226_r | 0.0501 | 0.1638 | −0.0666 | 0.2304 |
| HS_from_MQbrain_1102_l | 0.1296 | −0.2855 | −0.5159 | 0.2305 |
| HS_204_r | 0.1522 | −0.5105 | −0.7412 | 0.2306 |
| Hsd_27_r | 0.0895 | 0.3963 | 0.1656 | 0.2307 |
| HSbrain_12_l | 0.8957 | −0.0603 | −0.2920 | 0.2317 |
| hsa-mir-328_r | 0.1414 | −0.4172 | −0.6491 | 0.2319 |
| HS_from_MQbrain_1437_l | 0.1906 | −0.0115 | −0.2437 | 0.2323 |
| HS_from_MQbrain_745_r | 0.1887 | −0.0589 | −0.2916 | 0.2328 |
| HS_191_r | 0.1752 | −0.3362 | −0.5699 | 0.2336 |
| Hsd_from_Mmd_120_r | 0.1843 | −0.1897 | −0.4250 | 0.2353 |
| HSbrain_155_r | 0.1230 | 0.1793 | −0.0573 | 0.2366 |
| HS_from_MQbrain_1339_r | 0.1107 | −0.0438 | −0.2806 | 0.2367 |
| HSbrain_135_l | 0.0946 | 0.0917 | −0.1464 | 0.2382 |
| HSbrain_476_r | 0.0474 | 0.3016 | 0.0629 | 0.2386 |
| HS_from_MQbrain_1072_l | 0.0211 | 0.2237 | −0.0152 | 0.2389 |
| HS_from_MQbrain_914_l | 0.1447 | −0.5145 | −0.7543 | 0.2398 |
| HSbrain_1298_r | 0.0750 | −0.0314 | −0.2713 | 0.2399 |
| HS_from_PT_308_l | 0.0807 | 0.0766 | −0.1644 | 0.2410 |
| HS_from_MQbrain_433_l | 0.1045 | −0.2539 | −0.4951 | 0.2413 |
| HS_139_l | 0.1491 | −0.1113 | −0.3536 | 0.2422 |
| hsa-mir-138-2_l | 0.1997 | 0.0850 | −0.1584 | 0.2433 |
| HS_98_r | 0.3012 | −0.3911 | −0.6348 | 0.2436 |
| HS_from_MQbrain_1442_l | 0.1774 | 0.2928 | 0.0472 | 0.2456 |
| HS_from_MQbrain_1415_r | 0.1997 | −0.3303 | −0.5761 | 0.2458 |
| HSbrain_380_r | 0.1308 | −0.1815 | −0.4274 | 0.2459 |
| HS_171_r | 0.0946 | −0.0267 | −0.2742 | 0.2476 |
| HSbrain_726_r | 0.1390 | 0.0170 | −0.2306 | 0.2476 |
| HS_from_MQbrain_504_r | 0.0723 | −0.1830 | −0.4323 | 0.2493 |
| HS_from_PT_226_l | 0.0224 | 0.1161 | −0.1333 | 0.2494 |
| hsa-mir-584_r | 0.0857 | 0.0466 | −0.2029 | 0.2495 |
| HSbrain_256_r | 0.1230 | 0.8466 | 0.5964 | 0.2502 |
| HS_from_MQbrain_1185_l | 0.1131 | −0.4464 | −0.6973 | 0.2509 |
| HSbrain_703_r | 0.0772 | −0.2492 | −0.5002 | 0.2510 |
| HSbrain_91_r | 0.1597 | −0.3890 | −0.6401 | 0.2510 |
| HS_from_MQbrain_852_l | 0.1060 | 0.3566 | 0.1052 | 0.2514 |
| HS_from_MQbrain_868_l | 0.1616 | −0.1609 | −0.4128 | 0.2519 |
| HS_from_PT_157_r | 0.1858 | −0.3869 | −0.6391 | 0.2522 |
| HS_97_r | 0.1268 | 0.4079 | 0.1554 | 0.2524 |
| hsa-mir-640_l | 0.1216 | 0.1046 | −0.1481 | 0.2527 |
| HS_from_MQbrain_804_r | 0.1390 | −0.0330 | −0.2871 | 0.2541 |
| HS_from_MQbrain_1116_r | 0.1045 | −0.4187 | −0.6729 | 0.2542 |
| Hsd_from_Mmd_491_r | 0.0928 | 0.0936 | −0.1626 | 0.2562 |
| HSbrain_3_l | 0.1390 | 0.5138 | 0.2568 | 0.2570 |
| HS_from_MQbrain_1839_l | 0.1616 | −0.4341 | −0.6922 | 0.2581 |
| HS_from_MQbrain_1357_l | 0.1997 | −0.0965 | −0.3549 | 0.2584 |
| HS_from_MQbrain_1753_r | 0.1597 | 0.4978 | 0.2387 | 0.2590 |
| hsa-mir-222_r | 0.1906 | 0.2426 | −0.0186 | 0.2612 |
| HS_from_PT_128_l | 0.1159 | −0.1656 | −0.4278 | 0.2622 |
| HSbrain_441_r | 0.1196 | −0.2577 | −0.5199 | 0.2622 |
| HS_from_MM_219_l | 0.0913 | 0.1577 | −0.1047 | 0.2624 |
| HSbrain_703_l | 0.1010 | −0.1804 | −0.4430 | 0.2627 |
| HS_from_MQbrain_1893_r | 0.0983 | 0.4642 | 0.2015 | 0.2627 |
| HS_from_MQbrain_365_r | 0.0425 | 0.0118 | −0.2520 | 0.2638 |
| HS_from_MQbrain_1750_r | 0.0379 | −0.3565 | −0.6204 | 0.2639 |
| HS_from_MQbrain_1374_l | 0.1045 | −0.1628 | −0.4296 | 0.2668 |
| HSbrain_141_r | 0.1887 | −0.0643 | −0.3312 | 0.2669 |
| HSbrain_691_l | 0.5142 | −0.5974 | −0.8650 | 0.2676 |
| HS_from_MQbrain_814_l | 0.1243 | 0.3611 | 0.0916 | 0.2695 |
| HS_from_MQbrain_1426_r | 0.0075 | 0.2004 | −0.0691 | 0.2695 |
| HS_from_MQbrain_392_l | 0.0913 | 0.1809 | −0.0887 | 0.2696 |
| HS_from_MQbrain_1514_r | 0.1975 | −0.5681 | −0.8383 | 0.2703 |
| HS_from_MQbrain_996_l | 0.1078 | −0.0702 | −0.3413 | 0.2712 |
| HS_from_MQbrain_1882_r | 0.1597 | 0.5063 | 0.2350 | 0.2712 |
| HS_from_MQbrain_140_l | 0.1296 | 0.3493 | 0.0774 | 0.2719 |
| Hsd_from_Mmd_391_l | 0.0706 | 0.1694 | −0.1026 | 0.2720 |
| HS_from_MQbrain_597_l | 0.0895 | −0.3044 | −0.5765 | 0.2722 |
| HS_from_MQbrain_437_l | 0.1752 | −0.2897 | −0.5627 | 0.2730 |
| HSbrain_384_l | 0.0347 | 0.1094 | −0.1638 | 0.2732 |
| HS_from_MQbrain_984_l | 0.0393 | −0.1493 | −0.4234 | 0.2742 |
| HSbrain_497_l | 0.1887 | −0.5058 | −0.7804 | 0.2746 |
| HSbrain_1416_r | 0.1390 | −0.5420 | −0.8166 | 0.2746 |
| HS_from_MQbrain_163_l | 0.0501 | −0.3703 | −0.6453 | 0.2750 |
| HSbrain_43_r | 0.1268 | −0.0957 | −0.3712 | 0.2755 |
| Hsd_80_r | 0.1735 | −0.6000 | −0.8786 | 0.2786 |
| HS_from_PT_287_r | 0.0459 | 0.1585 | −0.1203 | 0.2788 |
| HS_from_MM_260_l | 0.1178 | 0.4785 | 0.1992 | 0.2793 |
| HSbrain_626_r | 0.1703 | −0.4710 | −0.7508 | 0.2798 |
| HS_from_MQbrain_1537_r | 0.1060 | −0.2818 | −0.5642 | 0.2824 |
| Hsd_from_Mmd_413_r | 0.0646 | −0.1419 | −0.4246 | 0.2827 |
| Hsd_from_Mmd_370_r | 0.1735 | 0.3001 | 0.0169 | 0.2832 |
| HS_154_r | 0.1216 | −0.4284 | −0.7119 | 0.2836 |
| HS_from_MM_276_l | 0.1146 | 0.0308 | −0.2531 | 0.2839 |
| Hsd_106_r | 0.1616 | 0.4018 | 0.1163 | 0.2855 |
| HSbrain_22_l | 0.1997 | 0.1279 | −0.1588 | 0.2867 |
| hsa-mir-125b-2_l | 0.1673 | 0.0719 | −0.2185 | 0.2904 |
| Hsd_from_Mmd_473_l | 0.0750 | 0.0392 | −0.2513 | 0.2905 |
| HS_from_MQbrain_1376_r | 0.1673 | 0.6193 | 0.3272 | 0.2921 |
| HS_from_MQbrain_1675_r | 0.0849 | −0.1068 | −0.3996 | 0.2929 |
| HS_from_MQbrain_1565_l | 0.0501 | 0.0842 | −0.2086 | 0.2929 |
| HS_from_MM_39_l | 0.0723 | 0.1386 | −0.1547 | 0.2934 |
| HSbrain_662_r | 0.1858 | 0.7747 | 0.4809 | 0.2938 |
| HS_from_MM_174_l | 0.0379 | −0.0310 | −0.3253 | 0.2943 |
| Hsd_from_Mmd_222_l | 0.0723 | 0.5247 | 0.2299 | 0.2947 |
| HSbrain_494_l | 0.6640 | 0.0672 | −0.2279 | 0.2951 |
| hsa-mir-524_l | 0.1997 | 0.2315 | −0.0642 | 0.2958 |
| HS_from_MQbrain_1006_l | 0.0723 | −0.2964 | −0.5932 | 0.2968 |
| HS_from_MM_275_r | 0.0772 | 0.1444 | −0.1526 | 0.2969 |
| HS_from_MQbrain_1444_l | 0.5661 | −0.2300 | −0.5274 | 0.2974 |
| HS_from_MQbrain_597_r | 0.0849 | −0.3089 | −0.6064 | 0.2975 |
| HSbrain_190_r | 0.1975 | 0.8006 | 0.5006 | 0.3001 |
| Hsd_from_Mmd_485_r | 0.0501 | −0.2526 | −0.5532 | 0.3006 |
| HSbrain_250_l | 0.1382 | −0.0881 | −0.3897 | 0.3015 |
| HS_from_MQbrain_697_l | 0.1382 | −0.2157 | −0.5181 | 0.3024 |
| HS_from_MQbrain_560_r | 0.1735 | −0.4285 | −0.7312 | 0.3027 |
| HS_from_MQbrain_629_r | 0.1555 | −0.6726 | −0.9758 | 0.3032 |
| HSbrain_465_l | 0.0946 | −0.2773 | −0.5814 | 0.3040 |
| HSbrain_252_l | 0.0528 | −0.1922 | −0.4989 | 0.3067 |
| HS_from_MQbrain_1817_l | 0.1482 | 0.6469 | 0.3397 | 0.3072 |
| HSbrain_795_r | 0.1146 | −0.3575 | −0.6648 | 0.3072 |
| Hsd_from_Mmd_117_r | 0.1026 | −0.2234 | −0.5320 | 0.3086 |
| HSbrain_1406_l | 0.0727 | 0.0109 | −0.2986 | 0.3095 |
| HSbrain_77_l | 0.0474 | −0.0601 | −0.3710 | 0.3109 |
| HS_from_MQbrain_71_l | 0.0582 | −0.3977 | −0.7098 | 0.3120 |
| HS_from_MQbrain_906_l | 0.1463 | −0.0012 | −0.3135 | 0.3124 |
| HSbrain_14_r | 0.1010 | 0.2815 | −0.0313 | 0.3128 |
| HSbrain_778_l | 0.1843 | 0.0025 | −0.3107 | 0.3132 |
| HSbrain_305_l | 0.1597 | −0.7574 | −1.0710 | 0.3136 |
| HSbrain_726_l | 0.1326 | −0.6046 | −0.9189 | 0.3143 |
| HSbrain_319_l | 0.0797 | 0.0842 | −0.2316 | 0.3159 |
| HS_from_MQbrain_178_r | 0.1390 | −0.3678 | −0.6837 | 0.3160 |
| HS_from_PT_338_l | 0.1673 | −0.3606 | −0.6772 | 0.3166 |
| HSbrain_438_r | 0.0475 | 0.2503 | −0.0664 | 0.3167 |
| HSbrain_764_r | 0.0857 | 0.2727 | −0.0448 | 0.3176 |
| HS_from_MM_118_r | 0.0446 | −0.2376 | −0.5555 | 0.3179 |
| HS_from_MQbrain_64_l | 0.1454 | 0.2943 | −0.0251 | 0.3193 |
| HS_175_l | 0.1454 | −0.5896 | −0.9091 | 0.3195 |
| HS_from_MQbrain_1877_l | 0.0913 | 0.0926 | −0.2269 | 0.3195 |
| HS_200_r | 0.0727 | −0.3033 | −0.6237 | 0.3204 |
| HSbrain_157_l | 0.1735 | −0.5154 | −0.8373 | 0.3219 |
| HS_from_MQbrain_777_r | 0.1131 | −0.1654 | −0.4873 | 0.3220 |
| HSbrain_437_r | 0.0501 | 0.2708 | −0.0515 | 0.3223 |
| HS_from_MQbrain_587_l | 0.1616 | −0.4611 | −0.7836 | 0.3226 |
| HS_from_MQbrain_1792_r | 0.0185 | 0.0272 | −0.2956 | 0.3228 |
| HSbrain_168_l | 0.1673 | −0.3405 | −0.6643 | 0.3238 |
| HS_from_MQbrain_399_l | 0.1390 | 0.2627 | −0.0624 | 0.3251 |
| Hsd_42_l | 0.0838 | −0.3226 | −0.6499 | 0.3272 |
| hsa-mir-632_l | 0.1243 | 0.3509 | 0.0234 | 0.3275 |
| HSbrain_719_l | 0.0995 | 0.3897 | 0.0612 | 0.3285 |
| HS_from_MQbrain_1623_r | 0.0849 | −0.1231 | −0.4520 | 0.3290 |
| HS_from_MQbrain_1085_l | 0.1010 | −0.6056 | −0.9354 | 0.3298 |
| HS_46_l | 0.0359 | −0.0381 | −0.3685 | 0.3304 |
| HS_from_MQbrain_235_r | 0.1997 | −0.4807 | −0.8115 | 0.3308 |
| HSbrain_571_l | 0.1597 | −0.5591 | −0.8905 | 0.3314 |
| HS_from_MQbrain_1633_r | 0.1243 | −0.3035 | −0.6354 | 0.3319 |
| HS_from_MQbrain_760_l | 0.1230 | −0.4651 | −0.7974 | 0.3323 |
| Hsd_from_Mmd_324_l | 0.0913 | −0.3968 | −0.7344 | 0.3376 |
| Hsd_from_Mmd_516_r | 0.1491 | 0.3870 | 0.0483 | 0.3387 |
| HS_from_MQbrain_603_r | 0.0446 | 0.1619 | −0.1777 | 0.3396 |
| Hsd_from_Mmd_452_l | 0.0459 | 0.0541 | −0.2865 | 0.3406 |
| HS_from_MM_118_l | 0.1482 | −0.5729 | −0.9150 | 0.3421 |
| HS_from_MQbrain_1817_l | 0.1482 | 0.3444 | 0.0015 | 0.3429 |
| HS_243_l | 0.1843 | 0.5174 | 0.1741 | 0.3433 |
| HS_181_l | 0.1616 | −0.7137 | −1.0574 | 0.3437 |
| HS_from_MQbrain_648_r | 0.1216 | −0.4144 | −0.7588 | 0.3444 |
| HS_71_r | 0.0144 | 0.1919 | −0.1534 | 0.3452 |
| HSbrain_1423_l | 0.0849 | −0.2372 | −0.5827 | 0.3455 |
| Hsd_66_l | 0.1522 | 0.8168 | 0.4707 | 0.3462 |
| HSbrain_306_r | 0.0239 | 0.1049 | −0.2415 | 0.3464 |
| Hsd_88_r | 0.0706 | 0.0816 | −0.2651 | 0.3466 |
| HS_from_MQbrain_1350_l | 0.0224 | 0.0909 | −0.2567 | 0.3476 |
| HSbrain_1325_r | 0.0409 | −0.1016 | −0.4506 | 0.3490 |
| HS_from_PT_250_l | 0.0369 | 0.5709 | 0.2216 | 0.3493 |
| HS_from_MQbrain_1627_r | 0.1703 | −0.5353 | −0.8861 | 0.3508 |
| HSbrain_512_l | 0.0895 | −0.5385 | −0.8915 | 0.3530 |
| Hsd_from_Mmd_464_l | 0.0764 | −0.1277 | −0.4807 | 0.3530 |
| HSbrain_442_r | 0.0797 | 0.5744 | 0.2202 | 0.3542 |
| HSbrain_1425_l | 0.0359 | −0.2671 | −0.6216 | 0.3544 |
| HS_from_MQbrain_129_r | 0.1026 | −0.3030 | −0.6587 | 0.3556 |
| HS_from_MQbrain_928_r | 0.0857 | −0.1851 | −0.5409 | 0.3558 |
| HSbrain_777_r | 0.1735 | −0.5204 | −0.8772 | 0.3567 |
| HS_from_MQbrain_184_l | 0.1146 | 0.7307 | 0.3738 | 0.3569 |
| HS_262_l | 0.0599 | 0.1304 | −0.2281 | 0.3584 |
| HS_from_MQbrain_765_l | 0.0913 | −0.2399 | −0.5992 | 0.3593 |
| HS_from_MQbrain_968_l | 0.1975 | −0.8313 | −1.1911 | 0.3598 |
| HS_from_MQbrain_634_r | 0.0838 | 0.5520 | 0.1913 | 0.3607 |
| HSbrain_7_r | 0.0347 | −0.2163 | −0.5775 | 0.3612 |
| HS_from_MQbrain_2669_l | 0.0474 | −0.1120 | −0.4735 | 0.3615 |
| HS_from_MQbrain_686_r | 0.0446 | −0.2735 | −0.6359 | 0.3624 |
| HS_from_MQbrain_822_l | 0.1268 | 0.5832 | 0.2203 | 0.3629 |
| HS_from_PT_308_l | 0.0807 | −0.0319 | −0.3958 | 0.3639 |
| HSbrain_393_r | 0.1296 | 0.2759 | −0.0882 | 0.3642 |
| HS_from_MQbrain_1827_l | 0.5863 | 0.2089 | −0.1554 | 0.3643 |
| Hsd_105_l | 0.0772 | −0.4763 | −0.8408 | 0.3645 |
| Hsd_from_Mmd_346_l | 0.1010 | −0.5632 | −0.9279 | 0.3647 |
| HS_from_MQbrain_346_l | 0.0599 | −0.1826 | −0.5479 | 0.3652 |
| HS_from_MQbrain_1806_l | 0.1243 | 0.6662 | 0.3008 | 0.3653 |
| HSbrain_93_r | 0.0723 | −0.0190 | −0.3846 | 0.3656 |
| HS_from_MQbrain_1840_l | 0.0995 | 0.3436 | −0.0251 | 0.3686 |
| Hsd_107_l | 0.0723 | −0.2032 | −0.5725 | 0.3693 |
| HS_from_MQbrain_1810_l | 0.1454 | 0.1897 | −0.1807 | 0.3705 |
| HSbrain_122_l | 0.0498 | −0.4275 | −0.7990 | 0.3715 |
| HS_from_MQbrain_1806_l | 0.1243 | 0.2919 | −0.0815 | 0.3734 |
| HS_from_MQbrain_1264_l | 0.0928 | 0.7104 | 0.3360 | 0.3744 |
| HSbrain_583_l | 0.0268 | −0.2068 | −0.5861 | 0.3793 |
| Hsd_69_l | 0.1382 | 0.6284 | 0.2466 | 0.3819 |
| HS_from_MQbrain_280_l | 0.1997 | 0.5624 | 0.1804 | 0.3820 |
| HS_from_MQbrain_314_l | 0.1268 | −0.5418 | −0.9241 | 0.3824 |
| HS_from_MQbrain_574_l | 0.1078 | 0.7104 | 0.3274 | 0.3830 |
| HS_299_l | 0.5233 | −0.0392 | −0.4238 | 0.3846 |
| HSbrain_389_l | 0.0849 | −0.5284 | −0.9148 | 0.3864 |
| HS_from_MQbrain_1250_l | 0.1997 | −0.4297 | −0.8163 | 0.3866 |
| HS_from_MQbrain_823_l | 0.0849 | 0.5688 | 0.1821 | 0.3867 |
| hsa-mir-591_r | 0.0501 | 0.4116 | 0.0245 | 0.3870 |
| HS_from_MQbrain_354_r | 0.1178 | −0.4911 | −0.8816 | 0.3905 |
| HS_from_PT_283_r | 0.1454 | −0.7717 | −1.1624 | 0.3907 |
| HSbrain_351_r | 0.0393 | −0.2481 | −0.6402 | 0.3921 |
| Hsd_from_Mmd_255_l | 0.1078 | −0.2783 | −0.6711 | 0.3928 |
| HS_from_MQbrain_1210_l | 0.1997 | 1.0100 | 0.6149 | 0.3951 |
| HS_from_MQbrain_2441_r | 0.1616 | 0.7908 | 0.3948 | 0.3960 |
| HS_from_MQbrain_37_r | 0.1308 | −0.7936 | −1.1910 | 0.3974 |
| HS_from_MQbrain_1268_l | 0.1673 | −0.8281 | −1.2273 | 0.3991 |
| hsa-mir-491_r | 0.1196 | −0.2543 | −0.6566 | 0.4023 |
| HS_from_MQbrain_43_r | 0.0393 | −0.1919 | −0.5962 | 0.4043 |
| hsa-mir-377_l | 0.0393 | 0.3186 | −0.0863 | 0.4049 |
| HS_from_MQbrain_377_l | 0.0169 | 0.2996 | −0.1079 | 0.4075 |
| HS_from_MM_242_r | 0.0142 | −0.1385 | −0.5463 | 0.4078 |
| HS_from_MQbrain_1087_l | 0.0838 | −0.3314 | −0.7403 | 0.4089 |
| hsa-mir-31_l | 0.1555 | 0.1679 | −0.2419 | 0.4098 |
| HS_from_MQbrain_845_r | 0.1414 | 0.2544 | −0.1576 | 0.4120 |
| HS_from_MQbrain_535_r | 0.1454 | 1.2319 | 0.8186 | 0.4133 |
| HS_from_MQbrain_1771_l | 0.0983 | −0.2405 | −0.6542 | 0.4138 |
| HS_from_MQbrain_1391_l | 0.0127 | −0.1673 | −0.5838 | 0.4165 |
| HSbrain_304_r | 0.0475 | −0.3663 | −0.7834 | 0.4171 |
| HS_from_MQbrain_534_r | 0.1060 | 0.0934 | −0.3256 | 0.4191 |
| HS_from_MQbrain_635_l | 0.1616 | −0.9169 | −1.3392 | 0.4223 |
| HS_from_MQbrain_1520_l | 0.1326 | −0.6739 | −1.0973 | 0.4233 |
| HS_from_PT_95_l | 0.1366 | −0.8666 | −1.2903 | 0.4236 |
| HS_from_MQbrain_1042_r | 0.0303 | 0.0247 | −0.3995 | 0.4242 |
| Hsd_from_Mmd_42_r | 0.1774 | −0.7209 | −1.1484 | 0.4275 |
| HSbrain_177_l | 0.0482 | −0.3234 | −0.7516 | 0.4282 |
| HS_from_MQbrain_1481_l | 0.0895 | 1.1221 | 0.6936 | 0.4285 |
| HS_from_MM_218_l | 0.0552 | −0.5267 | −0.9554 | 0.4287 |
| HS_from_MQbrain_301_l | 0.0727 | −0.6137 | −1.0427 | 0.4290 |
| hsa-mir-324_r | 0.0247 | 0.3174 | −0.1119 | 0.4293 |
| HS_14_r | 0.0928 | 0.4778 | 0.0470 | 0.4308 |
| HS_from_MQbrain_420_l | 0.0127 | 0.0841 | −0.3489 | 0.4329 |
| HS_from_MM_169_r | 0.0144 | −0.3087 | −0.7426 | 0.4339 |
| HS_from_MQbrain_1137_l | 0.0169 | −0.2359 | −0.6754 | 0.4395 |
| HSbrain_677_r | 0.1146 | −0.6569 | −1.0979 | 0.4410 |
| Hsd_41_r | 0.1997 | 0.9793 | 0.5380 | 0.4413 |
| HS_from_MQbrain_1500_l | 0.0239 | 0.6213 | 0.1785 | 0.4429 |
| HS_from_MQbrain_1783_r | 0.1060 | 0.7915 | 0.3475 | 0.4439 |
| hsa-mir-346_l | 0.0239 | 0.0099 | −0.4354 | 0.4453 |
| HS_from_MQbrain_634_l | 0.0928 | −0.9387 | −1.3843 | 0.4457 |
| HS_from_MQbrain_1246_l | 0.0727 | 0.8358 | 0.3894 | 0.4464 |
| HS_from_MQbrain_901_r | 0.0849 | 0.6231 | 0.1739 | 0.4492 |
| HS_276_l | 0.0169 | −0.0815 | −0.5325 | 0.4510 |
| HSbrain_198_l | 0.1491 | −0.8575 | −1.3085 | 0.4511 |
| HSbrain_202_r | 0.1597 | 0.3027 | −0.1517 | 0.4544 |
| HS_from_MQbrain_1778_r | 0.1858 | 1.0726 | 0.6180 | 0.4546 |
| HS_241_r | 0.1887 | 0.9239 | 0.4646 | 0.4593 |
| HSbrain_695_l | 0.0895 | −0.6791 | −1.1390 | 0.4599 |
| HS_from_MQbrain_590_l | 0.0156 | 0.1826 | −0.2798 | 0.4624 |
| Hsd_101_l | 0.0857 | −0.7444 | −1.2116 | 0.4672 |
| HS_from_MQbrain_281_r | 0.1366 | −0.9223 | −1.3963 | 0.4740 |
| HS_from_MQbrain_161_r | 0.1216 | 0.7005 | 0.2259 | 0.4747 |
| HS_from_MQbrain_1541_l | 0.1078 | 0.8304 | 0.3555 | 0.4749 |
| HS_from_MQbrain_65_l | 0.0683 | 0.3926 | −0.0826 | 0.4752 |
| hsa-mir-24-2_l | 0.0185 | −0.0415 | −0.5184 | 0.4769 |
| HSbrain_518_r | 0.0723 | 0.3909 | −0.0880 | 0.4789 |
| hsa-mir-661_r | 0.0475 | 0.0992 | −0.3802 | 0.4793 |
| HS_261_r | 0.0686 | −0.7602 | −1.2402 | 0.4800 |
| hsa-mir-383_r | 0.0473 | 0.4476 | −0.0344 | 0.4821 |
| HSbrain_47_r | 0.0750 | −0.5031 | −0.9862 | 0.4831 |
| HS_from_MQbrain_881_r | 0.0772 | 0.6213 | 0.1375 | 0.4839 |
| HSbrain_293_l | 0.0152 | −0.1832 | −0.6701 | 0.4869 |
| HS_from_MQbrain_825_r | 0.0264 | −0.4284 | −0.9154 | 0.4869 |
| HS_157_r | 0.0205 | −0.3871 | −0.8785 | 0.4914 |
| HS_from_MQbrain_1601_r | 0.1587 | 0.5820 | 0.0892 | 0.4928 |
| HSbrain_313_l | 0.1491 | −0.6644 | −1.1573 | 0.4929 |
| HSbrain_218_l | 0.1616 | −0.9500 | −1.4464 | 0.4964 |
| HSbrain_263_r | 0.1803 | 0.4141 | −0.0842 | 0.4982 |
| HS_from_MQbrain_736_r | 0.0715 | −0.6335 | −1.1319 | 0.4985 |
| HS_from_MQbrain_824_l | 0.0849 | −0.7246 | −1.2242 | 0.4995 |
| HSbrain_217_r | 0.0205 | −0.2148 | −0.7164 | 0.5017 |
| HSbrain_625_l | 0.1522 | 1.1058 | 0.6021 | 0.5037 |
| HSbrain_304_l | 0.1522 | 0.5220 | 0.0170 | 0.5051 |
| HS_136_r | 0.0142 | −0.2220 | −0.7315 | 0.5095 |
| HS_from_PT_36_l | 0.0127 | −0.2277 | −0.7409 | 0.5131 |
| HS_from_MQbrain_340_r | 0.1230 | 1.1696 | 0.6511 | 0.5185 |
| HS_from_PT_201_l | 0.0142 | −0.0537 | −0.5727 | 0.5190 |
| HS_from_MQbrain_1204_l | 0.0347 | −0.3953 | −0.9152 | 0.5199 |
| HS_38_r | 0.1146 | −0.8733 | −1.3976 | 0.5243 |
| HSbrain_334_r | 0.0156 | 0.1507 | −0.3766 | 0.5273 |
| HS_8_r | 0.0142 | 0.1320 | −0.4002 | 0.5322 |
| HSbrain_390_l | 0.0127 | −0.2812 | −0.8165 | 0.5353 |
| hsa-mir-619_l | 0.0764 | 0.1999 | −0.3361 | 0.5360 |
| HS_145_r | 0.0913 | −0.6142 | −1.1533 | 0.5391 |
| HS_from_MQbrain_1099_r | 0.0350 | −0.1187 | −0.6621 | 0.5434 |
| HSbrain_1363_r | 0.1491 | 1.1096 | 0.5657 | 0.5439 |
| HSbrain_570_r | 0.0347 | −0.4967 | −1.0440 | 0.5473 |
| Hsd_from_Mmd_286_r | 0.1803 | 1.3502 | 0.8009 | 0.5494 |
| hsa-mir-557_l | 0.1858 | 1.7522 | 1.2007 | 0.5515 |
| HS_289_r | 0.1366 | 1.0558 | 0.5014 | 0.5544 |
| HSbrain_325_r | 0.1196 | −0.4936 | −1.0526 | 0.5590 |
| HSbrain_553_r | 0.0983 | 0.6125 | 0.0529 | 0.5596 |
| HS_288_r | 0.0995 | 1.0441 | 0.4818 | 0.5623 |
| HSbrain_673_r | 0.0528 | −0.6094 | −1.1736 | 0.5642 |
| HSbrain_669_r | 0.0446 | −0.6296 | −1.1967 | 0.5671 |
| HSbrain_684_r | 0.0528 | −0.6646 | −1.2346 | 0.5700 |
| HS_from_MQbrain_921_r | 0.0723 | 0.3348 | −0.2353 | 0.5702 |
| HS_290_r | 0.1296 | 1.1141 | 0.5400 | 0.5740 |
| HS_from_MQbrain_932_l | 0.0683 | −0.7432 | −1.3291 | 0.5859 |
| HS_from_MQbrain_458_l | 0.0185 | −0.2287 | −0.8159 | 0.5872 |
| hsa-mir-136_l | 0.0473 | −0.4760 | −1.0693 | 0.5933 |
| HS_176_l | 0.0144 | −0.3321 | −0.9329 | 0.6008 |
| HSbrain_1362_r | 0.1159 | 1.0942 | 0.4901 | 0.6041 |
| HS_from_MM_339_l | 0.0749 | −0.5146 | −1.1191 | 0.6046 |
| HSbrain_1364_r | 0.1326 | 1.1234 | 0.5126 | 0.6108 |
| hsa-mir-345_r | 0.1382 | 1.1389 | 0.5259 | 0.6130 |
| HS_from_MQbrain_606_r | 0.1463 | 1.5231 | 0.9058 | 0.6173 |
| HS_from_MQbrain_386_r | 0.1774 | 0.9824 | 0.3650 | 0.6174 |
| HSbrain_680_r | 0.0314 | −0.6300 | −1.2537 | 0.6237 |
| HS_287_r | 0.1026 | 1.0883 | 0.4644 | 0.6239 |
| HSbrain_687_r | 0.0347 | −0.6327 | −1.2586 | 0.6259 |
| HS_from_MQbrain_453_r | 0.1858 | 1.9060 | 1.2728 | 0.6332 |
| HS_from_MQbrain_1463_l | 0.1752 | 1.5905 | 0.9464 | 0.6441 |
| Hsd_from_Mmd_246_r | 0.1463 | 0.0400 | −0.6096 | 0.6496 |
| HS_from_MQbrain_17_r | 0.1858 | 1.0675 | 0.4114 | 0.6561 |
| HS_from_MQbrain_638_l | 0.0075 | −0.3793 | −1.0461 | 0.6668 |
| HS_from_MQbrain_809_l | 0.1390 | 1.3436 | 0.6759 | 0.6676 |
| HS_231_l | 0.1752 | 1.9739 | 1.3049 | 0.6690 |
| HS_from_MQbrain_1168_l | 0.1587 | 1.3948 | 0.7208 | 0.6740 |
| HSbrain_429_l | 0.1752 | 1.8593 | 1.1848 | 0.6745 |
| Hsd_from_Mmd_266_r | 0.1107 | 0.1541 | −0.5262 | 0.6803 |
| HS_from_MQbrain_63_r | 0.0156 | −0.2442 | −0.9291 | 0.6849 |
| HS_from_MQbrain_973_l | 0.1463 | 1.6221 | 0.9293 | 0.6928 |
| HS_51_r | 0.0249 | −0.6916 | −1.3872 | 0.6956 |
| HS_from_MQbrain_808_l | 0.1646 | 1.4040 | 0.7031 | 0.7008 |
| HS_from_MQbrain_868_l | 0.1616 | 0.6606 | −0.0426 | 0.7031 |
| hsa-mir-517a_l | 0.1382 | 2.0127 | 1.3048 | 0.7079 |
| HSbrain_620_l | 0.1454 | 1.2829 | 0.5748 | 0.7081 |
| HSbrain_1360_r | 0.0824 | 1.1672 | 0.4558 | 0.7113 |
| HSbrain_484_l | 0.1347 | 0.4330 | −0.2891 | 0.7221 |
| HS_from_MM_35_r | 0.1943 | 2.2043 | 1.4807 | 0.7236 |
| Hsd_from_Mmd_35_r | 0.1735 | 2.2635 | 1.5337 | 0.7297 |
| HS_from_MQbrain_453_l | 0.1296 | 2.5221 | 1.7765 | 0.7455 |
| hsa-mir-517b_l | 0.1454 | 2.1153 | 1.3684 | 0.7469 |
| HS_from_MQbrain_800_l | 0.1296 | 1.4132 | 0.6626 | 0.7506 |
| HS_from_MQbrain_666_l | 0.1491 | 1.7216 | 0.9704 | 0.7511 |
| HS_from_MQbrain_1110_r | 0.1803 | 2.2951 | 1.5252 | 0.7699 |
| HS_from_MQbrain_1620_r | 0.0252 | −0.2132 | −0.9994 | 0.7862 |
| hsa-mir-517c_l | 0.1243 | 2.1320 | 1.3398 | 0.7922 |
| HSbrain_26_r | 0.1159 | 2.0135 | 1.2097 | 0.8037 |
| hsa-mir-520g_l | 0.1463 | 1.4240 | 0.6190 | 0.8050 |
| HSbrain_366_r | 0.1646 | 1.6560 | 0.8440 | 0.8120 |
| HS_from_MQbrain_1334_r | 0.0156 | −0.7830 | −1.6118 | 0.8288 |
| Hsd_13_l | 0.1390 | 1.8885 | 1.0496 | 0.8390 |
| HS_306_l | 0.1463 | 1.9410 | 1.0848 | 0.8561 |
| HS_306_l | 0.1463 | 1.9702 | 1.1069 | 0.8633 |
| HS_from_MQbrain_817_l | 0.1491 | 1.7886 | 0.9243 | 0.8643 |
| hsa-mir-671_l | 0.1366 | 1.9361 | 1.0647 | 0.8714 |
| HS_from_MQbrain_1082_l | 0.0183 | −0.8412 | −1.7267 | 0.8855 |
| HS_from_PT_313_l | 0.1308 | 1.9518 | 1.0482 | 0.9036 |
| hsa-mir-520h_l | 0.1347 | 1.5659 | 0.6398 | 0.9261 |
| Hsd_from_Mmd_94_l | 0.1308 | 0.8926 | −0.0531 | 0.9457 |
| HS_from_MQbrain_273_r | 0.1178 | 1.9679 | 1.0064 | 0.9615 |
| HS_from_MQbrain_722_r | 0.1045 | 1.4659 | 0.4622 | 1.0037 |

¹Wilcoxon analysis: cut off p value < 0.2
²Upregulated small RNA expression when ratio >1 (below line)
Downregulated small RNA expression when ratio <1 (above line)

TABLE 4c
Small RNAs that are upregulated in colorectal adenocarcinomas compared
to adenomas

HS_from_MQbrain_722_r

TABLE 4d
Small RNAs that are downregulated in colorectal adenocarcinomas
compared to adenomas

HS_101_l
HS_102_l
HS_105_l
HS_109_r
HS_110_r
HS_111_r
HS_113_l
HS_114_l
HS_118_l
HS_119_l
HS_124_r
HS_130_l
HS_133_l
HS_136_r
HS_138_l
HS_139_l
HS_14_r
HS_141_l
HS_142_l
HS_145_r
HS_151_r
HS_154_r
HS_157_r
HS_166_l
HS_167_r
HS_17_l
HS_171_r
HS_175_l
HS_176_l
HS_177_l
HS_181_l
HS_182_r
HS_189_l
HS_189_r
HS_190_r
HS_191_r
HS_200_r
HS_204_r
HS_207_l
HS_208_l
HS_208_r
HS_212_l
HS_213_l
HS_217_l
HS_219_l
HS_220_l
HS_223_r
HS_231_l
HS_237_r
HS_241_r
HS_243_l
HS_246_l
HS_261_r
HS_262_l
HS_265_l
HS_266_r
HS_276_l
HS_277_l
HS_278_l
HS_281_r
HS_281_r
HS_285_r
HS_286_r
HS_287_r
HS_288_l
HS_288_r
HS_289_r
HS_29_l
HS_290_r
HS_292_l
HS_292_r
HS_292_r
HS_299_l
HS_303_r
HS_306_l
HS_306_l
HS_307_r
HS_38_r
HS_39_r
HS_45_r
HS_46_l
HS_47_l
HS_49_r
HS_50_l
HS_50_r
HS_51_r
HS_54_l
HS_54_r
HS_56_l
HS_58_l
HS_61_r
HS_62_l
HS_66_r
HS_67_r
HS_71_r
HS_74_r
HS_76_r
HS_77_l
HS_77_r
HS_79_l
HS_8_r
HS_83_r
HS_86_r
HS_88_r
HS_91_l
HS_95_r
HS_97_r
HS_98_r
HS_99_r
HS_from_MM_1_l
HS_from_MM_102_r
HS_from_MM_103_l
HS_from_MM_118_l
HS_from_MM_118_r
HS_from_MM_119_r
HS_from_MM_121_l
HS_from_MM_124_l
HS_from_MM_131_l
HS_from_MM_136_l
HS_from_MM_150_r
HS_from_MM_157_l
HS_from_MM_169_r
HS_from_MM_174_l
HS_from_MM_18_l
HS_from_MM_18_r
HS_from_MM_180_r
HS_from_MM_184_r
HS_from_MM_190_l
HS_from_MM_198_r
HS_from_MM_199_l
HS_from_MM_200_l
HS_from_MM_208_r
HS_from_MM_218_l
HS_from_MM_219_l
HS_from_MM_232_r
HS_from_MM_242_r
HS_from_MM_251_r
HS_from_MM_256_r
HS_from_MM_260_l
HS_from_MM_275_r
HS_from_MM_276_l
HS_from_MM_288_l
HS_from_MM_295_l
HS_from_MM_310_l
HS_from_MM_311_l
HS_from_MM_32_r
HS_from_MM_331_r
HS_from_MM_339_l
HS_from_MM_34_l
HS_from_MM_342_r
HS_from_MM_35_r
HS_from_MM_39_l
HS_from_MM_48_l
HS_from_MM_6_r
HS_from_MM_72_r
HS_from_MM_80_l
HS_from_MM_84_l
HS_from_MM_84_r
HS_from_MM_86_l
HS_from_MM_92_r
HS_from_MQbrain_1001_r
HS_from_MQbrain_1006_l
HS_from_MQbrain_1031_r
HS_from_MQbrain_1042_r
HS_from_MQbrain_1070_r
HS_from_MQbrain_1072_l
HS_from_MQbrain_1075_l
HS_from_MQbrain_108_r
HS_from_MQbrain_1082_l
HS_from_MQbrain_1085_l
HS_from_MQbrain_1087_l
HS_from_MQbrain_1088_l
HS_from_MQbrain_1091_l
HS_from_MQbrain_1097_r
HS_from_MQbrain_1099_r
HS_from_MQbrain_1100_r
HS_from_MQbrain_1102_l
HS_from_MQbrain_111_l
HS_from_MQbrain_1110_l
HS_from_MQbrain_1110_r
HS_from_MQbrain_1116_r
HS_from_MQbrain_1126_l
HS_from_MQbrain_1127_r
HS_from_MQbrain_1134_r
HS_from_MQbrain_1137_r
HS_from_MQbrain_1143_l
HS_from_MQbrain_1159_l
HS_from_MQbrain_1164_l
HS_from_MQbrain_1164_r
HS_from_MQbrain_1168_l
HS_from_MQbrain_117_r
HS_from_MQbrain_1183_l
HS_from_MQbrain_1185_l
HS_from_MQbrain_120_l
HS_from_MQbrain_1204_l
HS_from_MQbrain_1204_r
HS_from_MQbrain_1210_l
HS_from_MQbrain_1212_l
HS_from_MQbrain_1215_l
HS_from_MQbrain_1224_r
HS_from_MQbrain_1227_l
HS_from_MQbrain_1227_l
HS_from_MQbrain_1229_r
HS_from_MQbrain_1235_l
HS_from_MQbrain_1246_l
HS_from_MQbrain_1250_l
HS_from_MQbrain_1253_r
HS_from_MQbrain_1260_r
HS_from_MQbrain_1261_r
HS_from_MQbrain_1263_r
HS_from_MQbrain_1264_l
HS_from_MQbrain_1268_l
HS_from_MQbrain_1272_l
HS_from_MQbrain_129_r
HS_from_MQbrain_1292_l
HS_from_MQbrain_1292_r
HS_from_MQbrain_1294_l
HS_from_MQbrain_1297_l
HS_from_MQbrain_1300_r
HS_from_MQbrain_1301_r
HS_from_MQbrain_1323_l
HS_from_MQbrain_133_r
HS_from_MQbrain_1331_r
HS_from_MQbrain_1334_r
HS_from_MQbrain_1339_r
HS_from_MQbrain_1340_l
HS_from_MQbrain_1346_r
HS_from_MQbrain_1347_r
HS_from_MQbrain_1350_l
HS_from_MQbrain_1354_r
HS_from_MQbrain_1355_r
HS_from_MQbrain_1357_l
HS_from_MQbrain_1363_l
HS_from_MQbrain_1365_l
HS_from_MQbrain_1372_l
HS_from_MQbrain_1374_l
HS_from_MQbrain_1374_r
HS_from_MQbrain_1376_r
HS_from_MQbrain_1391_l
HS_from_MQbrain_1394_l
HS_from_MQbrain_140_l
HS_from_MQbrain_1405_l
HS_from_MQbrain_1412_l
HS_from_MQbrain_1413_l
HS_from_MQbrain_1415_r
HS_from_MQbrain_1417_l
HS_from_MQbrain_1419_r
HS_from_MQbrain_1420_l
HS_from_MQbrain_1421_l
HS_from_MQbrain_1426_r
HS_from_MQbrain_1433_r
HS_from_MQbrain_1437_l
HS_from_MQbrain_1442_l
HS_from_MQbrain_1443_r
HS_from_MQbrain_1444_l
HS_from_MQbrain_1463_l
HS_from_MQbrain_1469_l
HS_from_MQbrain_1470_l
HS_from_MQbrain_1470_r
HS_from_MQbrain_1481_l
HS_from_MQbrain_1485_l
HS_from_MQbrain_1493_r
HS_from_MQbrain_1494_r
HS_from_MQbrain_1500_l
HS_from_MQbrain_1514_r
HS_from_MQbrain_1517_r
HS_from_MQbrain_1520_l
HS_from_MQbrain_1525_r
HS_from_MQbrain_1537_r
HS_from_MQbrain_154_r
HS_from_MQbrain_1541_l
HS_from_MQbrain_1545_r
HS_from_MQbrain_1549_l
HS_from_MQbrain_1556_r
HS_from_MQbrain_1565_l
HS_from_MQbrain_1569_l
HS_from_MQbrain_1593_r
HS_from_MQbrain_1601_r
HS_from_MQbrain_161_l
HS_from_MQbrain_161_r
HS_from_MQbrain_1620_r
HS_from_MQbrain_1623_r
HS_from_MQbrain_1627_r
HS_from_MQbrain_163_l
HS_from_MQbrain_1631_r
HS_from_MQbrain_1632_l
HS_from_MQbrain_1633_r
HS_from_MQbrain_1637_r
HS_from_MQbrain_1645_l
HS_from_MQbrain_1649_r
HS_from_MQbrain_1657_r
HS_from_MQbrain_1667_r
HS_from_MQbrain_1675_l
HS_from_MQbrain_1675_r
HS_from_MQbrain_1677_r
HS_from_MQbrain_1684_r
HS_from_MQbrain_17_r
HS_from_MQbrain_1700_r
HS_from_MQbrain_1708_l
HS_from_MQbrain_1717_r
HS_from_MQbrain_1722_l
HS_from_MQbrain_1725_l
HS_from_MQbrain_1740_r
HS_from_MQbrain_1747_l
HS_from_MQbrain_1748_r
HS_from_MQbrain_1750_r
HS_from_MQbrain_1752_l
HS_from_MQbrain_1753_r
HS_from_MQbrain_1756_l
HS_from_MQbrain_1771_l
HS_from_MQbrain_1771_r
HS_from_MQbrain_1773_l
HS_from_MQbrain_1776_r
HS_from_MQbrain_1777_l
HS_from_MQbrain_1778_r
HS_from_MQbrain_178_r
HS_from_MQbrain_1781_l
HS_from_MQbrain_1783_r
HS_from_MQbrain_1792_r
HS_from_MQbrain_1793_r
HS_from_MQbrain_180_r
HS_from_MQbrain_1806_l
HS_from_MQbrain_1806_l
HS_from_MQbrain_1808_r
HS_from_MQbrain_181_l
HS_from_MQbrain_1810_l
HS_from_MQbrain_1817_l
HS_from_MQbrain_1817_l
HS_from_MQbrain_1824_l
HS_from_MQbrain_1827_l
HS_from_MQbrain_1832_l
HS_from_MQbrain_1836_l
HS_from_MQbrain_1839_l
HS_from_MQbrain_184_l
HS_from_MQbrain_1840_l
HS_from_MQbrain_1845_l
HS_from_MQbrain_1855_r
HS_from_MQbrain_1876_l
HS_from_MQbrain_1877_l
HS_from_MQbrain_1877_r
HS_from_MQbrain_188_l
HS_from_MQbrain_1882_r
HS_from_MQbrain_1893_r
HS_from_MQbrain_1896_r
HS_from_MQbrain_1901_l
HS_from_MQbrain_194_l
HS_from_MQbrain_1958_l
HS_from_MQbrain_1985_l
HS_from_MQbrain_2087_r
HS_from_MQbrain_209_r
HS_from_MQbrain_210_r
HS_from_MQbrain_212_r
HS_from_MQbrain_216_l
HS_from_MQbrain_22_l
HS_from_MQbrain_22_r
HS_from_MQbrain_221_r
HS_from_MQbrain_229_l
HS_from_MQbrain_235_r
HS_from_MQbrain_2441_r
HS_from_MQbrain_266_l
HS_from_MQbrain_266_r
HS_from_MQbrain_2669_l
HS_from_MQbrain_2670_r
HS_from_MQbrain_268_r
HS_from_MQbrain_272_l
HS_from_MQbrain_273_r
HS_from_MQbrain_280_l
HS_from_MQbrain_281_r
HS_from_MQbrain_283_r
HS_from_MQbrain_288_r
HS_from_MQbrain_29_l
HS_from_MQbrain_301_l
HS_from_MQbrain_314_l
HS_from_MQbrain_315_l
HS_from_MQbrain_330_r
HS_from_MQbrain_332_l
HS_from_MQbrain_340_r
HS_from_MQbrain_346_l
HS_from_MQbrain_348_r
HS_from_MQbrain_354_r
HS_from_MQbrain_355_l
HS_from_MQbrain_36_r
HS_from_MQbrain_365_r
HS_from_MQbrain_37_r
HS_from_MQbrain_375_l
HS_from_MQbrain_377_l
HS_from_MQbrain_38_r
HS_from_MQbrain_382_l
HS_from_MQbrain_386_r
HS_from_MQbrain_392_l
HS_from_MQbrain_397_l
HS_from_MQbrain_399_l
HS_from_MQbrain_401_l
HS_from_MQbrain_405_r
HS_from_MQbrain_42_l
HS_from_MQbrain_420_l
HS_from_MQbrain_429_l
HS_from_MQbrain_43_r
HS_from_MQbrain_433_l
HS_from_MQbrain_435_r
HS_from_MQbrain_437_l
HS_from_MQbrain_441_l
HS_from_MQbrain_444_r
HS_from_MQbrain_453_l
HS_from_MQbrain_453_r
HS_from_MQbrain_458_l
HS_from_MQbrain_467_l
HS_from_MQbrain_468_r
HS_from_MQbrain_470_l
HS_from_MQbrain_482_l
HS_from_MQbrain_496_r
HS_from_MQbrain_500_r
HS_from_MQbrain_501_l
HS_from_MQbrain_504_r
HS_from_MQbrain_529_r
HS_from_MQbrain_534_l
HS_from_MQbrain_534_r
HS_from_MQbrain_535_r
HS_from_MQbrain_536_r
HS_from_MQbrain_542_r
HS_from_MQbrain_55_l
HS_from_MQbrain_550_l
HS_from_MQbrain_555_r
HS_from_MQbrain_56_l
HS_from_MQbrain_560_r
HS_from_MQbrain_566_l
HS_from_MQbrain_57_l
HS_from_MQbrain_574_l
HS_from_MQbrain_579_l
HS_from_MQbrain_58_l
HS_from_MQbrain_587_l
HS_from_MQbrain_590_l
HS_from_MQbrain_597_l
HS_from_MQbrain_597_r
HS_from_MQbrain_600_l
HS_from_MQbrain_603_r
HS_from_MQbrain_604_l
HS_from_MQbrain_606_r
HS_from_MQbrain_609_l
HS_from_MQbrain_619_l
HS_from_MQbrain_62_l
HS_from_MQbrain_626_r
HS_from_MQbrain_628_l
HS_from_MQbrain_629_r
HS_from_MQbrain_63_r
HS_from_MQbrain_631_l
HS_from_MQbrain_634_l
HS_from_MQbrain_634_r
HS_from_MQbrain_635_l
HS_from_MQbrain_635_r
HS_from_MQbrain_638_l
HS_from_MQbrain_64_l
HS_from_MQbrain_648_r
HS_from_MQbrain_65_l
HS_from_MQbrain_653_l
HS_from_MQbrain_660_l
HS_from_MQbrain_661_r
HS_from_MQbrain_664_r
HS_from_MQbrain_666_l
HS_from_MQbrain_672_l
HS_from_MQbrain_675_l
HS_from_MQbrain_681_l
HS_from_MQbrain_686_l
HS_from_MQbrain_686_r
HS_from_MQbrain_691_r
HS_from_MQbrain_697_l
HS_from_MQbrain_699_r
HS_from_MQbrain_70_l
HS_from_MQbrain_71_l
HS_from_MQbrain_710_r
HS_from_MQbrain_715_l
HS_from_MQbrain_716_l
HS_from_MQbrain_72_r
HS_from_MQbrain_722_l
HS_from_MQbrain_723_l
HS_from_MQbrain_734_l
HS_from_MQbrain_736_r
HS_from_MQbrain_74_r
HS_from_MQbrain_745_r
HS_from_MQbrain_760_l
HS_from_MQbrain_765_l
HS_from_MQbrain_770_r
HS_from_MQbrain_777_r
HS_from_MQbrain_789_r
HS_from_MQbrain_791_r
HS_from_MQbrain_800_l
HS_from_MQbrain_804_r
HS_from_MQbrain_808_l
HS_from_MQbrain_809_l
HS_from_MQbrain_81_l
HS_from_MQbrain_812_l
HS_from_MQbrain_814_l
HS_from_MQbrain_817_l
HS_from_MQbrain_819_r
HS_from_MQbrain_822_l
HS_from_MQbrain_823_l
HS_from_MQbrain_824_l
HS_from_MQbrain_824_r
HS_from_MQbrain_825_r
HS_from_MQbrain_828_l
HS_from_MQbrain_833_l
HS_from_MQbrain_842_l
HS_from_MQbrain_845_r
HS_from_MQbrain_85_l
HS_from_MQbrain_852_l
HS_from_MQbrain_854_r
HS_from_MQbrain_859_l
HS_from_MQbrain_868_l
HS_from_MQbrain_868_l
HS_from_MQbrain_869_l
HS_from_MQbrain_88_l
HS_from_MQbrain_881_r
HS_from_MQbrain_886_r
HS_from_MQbrain_897_r
HS_from_MQbrain_90_r
HS_from_MQbrain_900_r
HS_from_MQbrain_901_r
HS_from_MQbrain_906_l
HS_from_MQbrain_914_l
HS_from_MQbrain_914_l
HS_from_MQbrain_921_l
HS_from_MQbrain_921_r
HS_from_MQbrain_928_r
HS_from_MQbrain_93_r
HS_from_MQbrain_932_l
HS_from_MQbrain_936_l
HS_from_MQbrain_940_r
HS_from_MQbrain_941_r
HS_from_MQbrain_951_l
HS_from_MQbrain_968_l
HS_from_MQbrain_968_r
HS_from_MQbrain_969_l
HS_from_MQbrain_97_r
HS_from_MQbrain_972_r
HS_from_MQbrain_973_l
HS_from_MQbrain_974_l
HS_from_MQbrain_984_l
HS_from_MQbrain_991_r
HS_from_MQbrain_996_l
HS_from_PT_101_r
HS_from_PT_115_r
HS_from_PT_117_r
HS_from_PT_126_l
HS_from_PT_128_l
HS_from_PT_13_l
HS_from_PT_134_l
HS_from_PT_139_r
HS_from_PT_140_l
HS_from_PT_149_r
HS_from_PT_157_r
HS_from_PT_187_r
HS_from_PT_188_l
HS_from_PT_190_r
HS_from_PT_196_l
HS_from_PT_20_r
HS_from_PT_201_l
HS_from_PT_211_r
HS_from_PT_226_l
HS_from_PT_237_r
HS_from_PT_238_l
HS_from_PT_250_l
HS_from_PT_254_l
HS_from_PT_254_r
HS_from_PT_27_l
HS_from_PT_27_r
HS_from_PT_282_l
HS_from_PT_283_r
HS_from_PT_287_r
HS_from_PT_299_r
HS_from_PT_308_l
HS_from_PT_308_l
HS_from_PT_313_l
HS_from_PT_338_l
HS_from_PT_34_r
HS_from_PT_36_l
HS_from_PT_41_l
HS_from_PT_44_l
HS_from_PT_60_l
HS_from_PT_91_r
HS_from_PT_95_l
hsa-let-7a-1_r
hsa-Iet-7c_r
hsa-let-7f-1_r
hsa-mir-10b_r
hsa-mir-1-1_l
hsa-mir-124a-1_l
hsa-mir-125b-2_l
hsa-mir-130b_l
hsa-mir-132_l
hsa-mir-133a-1_r
hsa-mir-135a-2_r
hsa-mir-136_l
hsa-mir-138-2_l
hsa-mir-146a_r
hsa-mir-148a_l
hsa-mir-155_r
hsa-mir-16-1_r
hsa-mir-190_l
hsa-mir-190_r
hsa-mir-191_r
hsa-mir-193a_l
hsa-mir-196a-2_r
hsa-mir-204_l
hsa-mir-204_r
hsa-mir-206_l
hsa-mir-217_r
hsa-mir-219-1_r
hsa-mir-222_r
hsa-mir-224_r
hsa-mir-24-2_l
hsa-mir-296_l
hsa-mir-299_l
hsa-mir-299_r
hsa-mir-301_l
hsa-mir-30b_l
hsa-mir-30b_r
hsa-mir-30c-1_l
hsa-mir-31_l
hsa-mir-324_r
hsa-mir-328_r
hsa-mir-329-1_r
hsa-mir-331_l
hsa-mir-338_l
hsa-mir-345_r
hsa-mir-346_l
hsa-mir-362_r
hsa-mir-365-1_l
hsa-mir-365-2_l
hsa-mir-365-2_r
hsa-mir-367_r
hsa-mir-368_l
hsa-mir-369_l
hsa-mir-374_l
hsa-mir-375_r
hsa-mir-377_l
hsa-mir-382_r
hsa-mir-383_r
hsa-mir-411_r
hsa-mir-412_l
hsa-mir-412_r
hsa-mir-433_r
hsa-mir-450-2_r
hsa-mir-451_l
hsa-mir-453_l
hsa-mir-454_l
hsa-mir-454_r
hsa-mir-488_l
hsa-mir-491_r
hsa-mir-497_r
hsa-mir-502_l
hsa-mir-510_r
hsa-mir-516-1_r
hsa-mir-516-2_r
hsa-mir-517a_l
hsa-mir-517b_l
hsa-mir-517c_l
hsa-mir-518e_r
hsa-mir-520f_r
hsa-mir-520g_l
hsa-mir-520h_l
hsa-mir-523_r
hsa-mir-524_l
hsa-mir-524_r
hsa-mir-548b_l
hsa-mir-548c_l
hsa-mir-550-2_r
hsa-mir-557_l
hsa-mir-568_l
hsa-mir-573_r
hsa-mir-579_r
hsa-mir-584_r
hsa-mir-588_r
hsa-mir-591_r
hsa-mir-593_r
hsa-mir-604_l
hsa-mir-616_l
hsa-mir-616_r
hsa-mir-619_l
hsa-mir-622_l
hsa-mir-623_r
hsa-mir-624_l
hsa-mir-625_r
hsa-mir-632_l
hsa-mir-633_r
hsa-mir-638_r
hsa-mir-640_l
hsa-mir-643_r
hsa-mir-646_r
hsa-mir-647_l
hsa-mir-648_l
hsa-mir-652_r
hsa-mir-653_l
hsa-mir-655_l
hsa-mir-659_l
hsa-mir-661_r
hsa-mir-671_l
hsa-mir-767_r
hsa-mir-770_r
hsa-mir-95_r
hsa-mir-98_r
HSbrain_102_r
HSbrain_106_r
HSbrain_1076_r
HSbrain_111_l
HSbrain_113_r
HSbrain_114_r
HSbrain_117_r
HSbrain_12_l
HSbrain_122_l
HSbrain_123_l
HSbrain_1298_r
HSbrain_1325_r
HSbrain_1331_r
HSbrain_1337_l
HSbrain_1338_r
HSbrain_135_l
HSbrain_1360_r
HSbrain_1362_r
HSbrain_1363_r
HSbrain_1364_l
HSbrain_1364_r
HSbrain_1377_l
HSbrain_1384_r
HSbrain_139_r
HSbrain_14_r
HSbrain_1406_l
HSbrain_141_r
HSbrain_1413_l
HSbrain_1414_r
HSbrain_1415_r
HSbrain_1416_r
HSbrain_1423_l
HSbrain_1425_l
HSbrain_155_r
HSbrain_157_l
HSbrain_158_l
HSbrain_16_l
HSbrain_166_l
HSbrain_168_l
HSbrain_174_r
HSbrain_177_l
HSbrain_177_r
HSbrain_180_l
HSbrain_182_r
HSbrain_184_r
HSbrain_187_r
HSbrain_190_r
HSbrain_198_l
HSbrain_202_r
HSbrain_204_r
HSbrain_210_l
HSbrain_214_l
HSbrain_217_r
HSbrain_218_l
HSbrain_22_l
HSbrain_220_l
HSbrain_220_r
HSbrain_222_r
HSbrain_225_r
HSbrain_226_r
HSbrain_231_l
HSbrain_235_l
HSbrain_236_l
HSbrain_244_l
HSbrain_25_r
HSbrain_250_l
HSbrain_250_r
HSbrain_252_l
HSbrain_256_r
HSbrain_259_r
HSbrain_26_r
HSbrain_263_r
HSbrain_266_r
HSbrain_270_r
HSbrain_272_l
HSbrain_276_l
HSbrain_289_r
HSbrain_293_l
HSbrain_3_l
HSbrain_304_l
HSbrain_304_r
HSbrain_305_l
HSbrain_305_r
HSbrain_306_r
HSbrain_307_r
HSbrain_313_l
HSbrain_316_l
HSbrain_319_l
HSbrain_32_l
HSbrain_325_r
HSbrain_328_l
HSbrain_334_r
HSbrain_336_l
HSbrain_337_r
HSbrain_347_l
HSbrain_351_r
HSbrain_359_l
HSbrain_361_l
HSbrain_365_l
HSbrain_366_r
HSbrain_369_r
HSbrain_371_r
HSbrain_374_r
HSbrain_380_r
HSbrain_381_l
HSbrain_384_l
HSbrain_388_r
HSbrain_389_l
HSbrain_390_l
HSbrain_393_l
HSbrain_393_r
HSbrain_399_l
HSbrain_402_l
HSbrain_408_r
HSbrain_41_l
HSbrain_419_l
HSbrain_420_r
HSbrain_425_l
HSbrain_429_l
HSbrain_43_r
HSbrain_431_l
HSbrain_431_r
HSbrain_437_r
HSbrain_438_r
HSbrain_440_l
HSbrain_441_r
HSbrain_442_r
HSbrain_452_l
HSbrain_461_r
HSbrain_465_l
HSbrain_468_r
HSbrain_47_r
HSbrain_472_l
HSbrain_473_l
HSbrain_476_r
HSbrain_479_l
HSbrain_480_r
HSbrain_482_l
HSbrain_483_r
HSbrain_484_l
HSbrain_494_l
HSbrain_495_r
HSbrain_497_l
HSbrain_50_l
HSbrain_50_r
HSbrain_506_r
HSbrain_507_l
HSbrain_509_l
HSbrain_512_l
HSbrain_513_r
HSbrain_518_r
HSbrain_52_r
HSbrain_532_l
HSbrain_538_r
HSbrain_540_l
HSbrain_541_r
HSbrain_545_r
HSbrain_55_l
HSbrain_553_r
HSbrain_568_r
HSbrain_570_l
HSbrain_570_r
HSbrain_571_l
HSbrain_578_r
HSbrain_580_r
HSbrain_583_l
HSbrain_583_r
HSbrain_588_l
HSbrain_588_r
HSbrain_590_l
HSbrain_591_l
HSbrain_593_r
HSbrain_594_l
HSbrain_599_r
HSbrain_6_r
HSbrain_606_l
HSbrain_614_l
HSbrain_62_r
HSbrain_620_l
HSbrain_621_l
HSbrain_621_r
HSbrain_622_r
HSbrain_624_l
HSbrain_625_l
HSbrain_626_r
HSbrain_627_l
HSbrain_627_r
HSbrain_636_l
HSbrain_643_l
HSbrain_644_r
HSbrain_647_r
HSbrain_651_r
HSbrain_652_l
HSbrain_661_l
HSbrain_662_r
HSbrain_663_l
HSbrain_669_r
HSbrain_673_l
HSbrain_673_r
HSbrain_677_l
HSbrain_677_r
HSbrain_680_r
HSbrain_684_r
HSbrain_687_l
HSbrain_687_r
HSbrain_689_r
HSbrain_690_l
HSbrain_691_l
HSbrain_692_l
HSbrain_692_r
HSbrain_695_l
HSbrain_696_r
HSbrain_698_r
HSbrain_7_r
HSbrain_703_l
HSbrain_703_r
HSbrain_705_l
HSbrain_708_l
HSbrain_716_l
HSbrain_717_l
HSbrain_717_r
HSbrain_719_l
HSbrain_72_l
HSbrain_720_l
HSbrain_726_l
HSbrain_726_r
HSbrain_728_l
HSbrain_744_l
HSbrain_75_l
HSbrain_753_r
HSbrain_754_r
HSbrain_755_l
HSbrain_755_r
HSbrain_757_r
HSbrain_764_r
HSbrain_77_l
HSbrain_771_l
HSbrain_773_r
HSbrain_776_l
HSbrain_777_r
HSbrain_778_l
HSbrain_779_r
HSbrain_780_r
HSbrain_783_l
HSbrain_787_l
HSbrain_791_l
HSbrain_792_r
HSbrain_794_l
HSbrain_794_r
HSbrain_795_r
HSbrain_796_r
HSbrain_799_r
HSbrain_80_l
HSbrain_803_l
HSbrain_809_r
HSbrain_811_l
HSbrain_82_r
HSbrain_85_r
HSbrain_91_r
HSbrain_916_l
HSbrain_93_r
HSbrain_95_l
HSbrain_970_l
HSbrain_970_r
Hsd_101_l
Hsd_105_l
Hsd_106_l
Hsd_106_r
Hsd_107_l
Hsd_108_l
Hsd_12_r
Hsd_13_l
Hsd_18_r
Hsd_24_r
Hsd_27_r
Hsd_30_l
Hsd_32_r
Hsd_37_r
Hsd_41_r
Hsd_42_l
Hsd_42_r
Hsd_46_l
Hsd_62_l
Hsd_66_l
Hsd_69_l
Hsd_7_r
Hsd_70_r
Hsd_8_r
Hsd_80_r
Hsd_81_r
Hsd_82_r
Hsd_83_l
Hsd_84_r
Hsd_85_r
Hsd_88_r
Hsd_95_l
Hsd_96_r
Hsd_from_Mmd_107_r
Hsd_from_Mmd_117_r
Hsd_from_Mmd_120_r
Hsd_from_Mmd_123_r
Hsd_from_Mmd_136_l
Hsd_from_Mmd_138_l
Hsd_from_Mmd_154_l
Hsd_from_Mmd_155_l
Hsd_from_Mmd_157_r
Hsd_from_Mmd_163_r
Hsd_from_Mmd_166_l
Hsd_from_Mmd_166_r
Hsd_from_Mmd_167_r
Hsd_from_Mmd_176_r
Hsd_from_Mmd_178_l
Hsd_from_Mmd_181_r
Hsd_from_Mmd_189_l
Hsd_from_Mmd_197_l
Hsd_from_Mmd_215_r
Hsd_from_Mmd_217_r
Hsd_from_Mmd_222_l
Hsd_from_Mmd_223_r
Hsd_from_Mmd_225_l
Hsd_from_Mmd_23_l
Hsd_from_Mmd_237_l
Hsd_from_Mmd_246_r
Hsd_from_Mmd_253_l
Hsd_from_Mmd_255_l
Hsd_from_Mmd_261_l
Hsd_from_Mmd_261_r
Hsd_from_Mmd_266_r
Hsd_from_Mmd_286_r
Hsd_from_Mmd_302_r
Hsd_from_Mmd_314_r
Hsd_from_Mmd_324_l
Hsd_from_Mmd_330_l
Hsd_from_Mmd_346_l
Hsd_from_Mmd_347_l
Hsd_from_Mmd_35_r
Hsd_from_Mmd_370_r
Hsd_from_Mmd_371_r
Hsd_from_Mmd_391_l
Hsd_from_Mmd_396_l
Hsd_from_Mmd_398_r
Hsd_from_Mmd_405_l
Hsd_from_Mmd_410_r
Hsd_from_Mmd_413_r
Hsd_from_Mmd_42_r
Hsd_from_Mmd_422_l
Hsd_from_Mmd_424_l
Hsd_from_Mmd_44_r
Hsd_from_Mmd_443_l
Hsd_from_Mmd_452_l
Hsd_from_Mmd_464_l
Hsd_from_Mmd_471_r
Hsd_from_Mmd_473_l
Hsd_from_Mmd_478_l
Hsd_from_Mmd_48_r
Hsd_from_Mmd_480_l
Hsd_from_Mmd_480_r
Hsd_from_Mmd_485_r
Hsd_from_Mmd_487_r
Hsd_from_Mmd_49_l
Hsd_from_Mmd_491_r
Hsd_from_Mmd_500_l
Hsd_from_Mmd_501_l
Hsd_from_Mmd_503_l
Hsd_from_Mmd_509_l
Hsd_from_Mmd_511_r
Hsd_from_Mmd_516_r
Hsd_from_Mmd_52_r
Hsd_from_Mmd_524_l
Hsd_from_Mmd_527_l
Hsd_from_Mmd_530_l
Hsd_from_Mmd_535_r
Hsd_from_Mmd_58_r
Hsd_from_Mmd_70_l
Hsd_from_Mmd_81_r
Hsd_from_Mmd_82_r
Hsd_from_Mmd_93_r
Hsd_from_Mmd_94_l
Hsd_from_Mmd_97_l

TABLE 5a
List of small RNAs as good classifiers to distinguish colorectal control
from adenoma tissue

| Systematic name | adenoma score | control score |
|---|---|---|
| [1,] HSbrain_377_l | 0.1008 | −0.3695 |
| [2,] HS_from_PT_114_r | 0.0617 | −0.2261 |
| [3,] HSbrain_377_r | 0.0606 | −0.2224 |
| [4,] hsa-mir-124a-3_r | −0.0447 | 0.164 |
| [5,] HSbrain_411_r | 0.0443 | −0.1624 |
| [6,] HS_from_MM_118_r | −0.0416 | 0.1524 |
| [7,] HSbrain_398_r | 0.0371 | −0.1359 |
| [8,] hsa-mir-421_r | 0.0368 | −0.1348 |
| [9,] HS_291_r | 0.0361 | −0.1322 |
| [10,] HS_from_MM_169_r | −0.0342 | 0.1255 |
| [11,] hsa-mir-124a-2_r | −0.0332 | 0.1218 |
| [12,] HSbrain_458_r | −0.033 | 0.121 |
| [13,] HS_from_PT_114_l | 0.0323 | −0.1183 |
| [14,] HS_from_MQbrain_594_r | −0.0317 | 0.1161 |
| [15,] HS_from_PT_36_l | −0.0308 | 0.1131 |
| [16,] hsa-mir-92-1_r | 0.0306 | −0.1123 |
| [17,] HSbrain_1437_r | 0.0274 | −0.1005 |
| [18,] hsa-mir-27a_r | 0.027 | −0.0992 |
| [19,] hsa-mir-124a-1_r | −0.0266 | 0.0975 |
| [20,] HS_from_MQbrain_2700_r | 0.0245 | −0.0897 |
| [21,] Hsd_from_Mmd_298_r | 0.0242 | −0.0887 |
| [22,] hsa-mir-92-2_r | 0.0234 | −0.0858 |
| [23,] HSbrain_52_r | 0.0227 | −0.0832 |
| [24,] Hsd_from_Mmd_485_r | −0.021 | 0.077 |
| [25,] HSbrain_1406_l | −0.0205 | 0.0751 |
| [26,] Hsd_from_Mmd_452_l | −0.0185 | 0.0679 |
| [27,] Hsd_from_Mmd_117_r | −0.0182 | 0.0666 |
| [28,] HS_97_r | −0.0174 | 0.0639 |
| [29,] HS_from_MQbrain_79_r | 0.0171 | −0.0628 |
| [30,] hsa-mir-635_l | −0.0164 | 0.06 |
| [31,] HSbrain_106_r | 0.0147 | −0.0538 |
| [32,] HS_262_l | −0.0136 | 0.05 |
| [33,] HS_from_MQbrain_1311_r | 0.0135 | −0.0496 |
| [34,] HS_291_r | 0.0132 | −0.0486 |
| [35,] HS_from_MQbrain_1462_l | 0.013 | −0.0477 |
| [36,] hsa-mir-130a_r | 0.0114 | −0.042 |
| [37,] HSbrain_500_r | 0.0111 | −0.0408 |
| [38,] hsa-mir-768_r | 0.0109 | −0.04 |
| [39,] HS_from_MQbrain_1183_l | 0.0108 | −0.0396 |
| [40,] HSbrain_224_r | 0.01 | −0.0365 |
| [41,] hsa-mir-29a_r | 0.0094 | −0.0344 |
| [42,] HSbrain_398_l | 0.0078 | −0.0285 |
| [43,] HS_71_l | −0.0073 | 0.0267 |
| [44,] HS_169_l | 0.0068 | −0.025 |
| [45,] hsa-mir-181c_l | 0.0066 | −0.0243 |
| [46,] HSbrain_500_l | 0.0038 | −0.0139 |
| [47,] HS_from_MQbrain_825_r | −0.0038 | 0.0138 |
| [48,] hsa-mir-146a_l | 0.0036 | −0.0132 |
| [49,] hsa-mir-454_r | 0.0034 | −0.0124 |
| [50,] HSbrain_1_l | 0.0025 | −0.0091 |
| [51,] HS_from_MQbrain_1823_r | 0.0025 | −0.009 |
| [52,] hsa-mir-145_l | 0.0014 | −0.0052 |
| [53,] HS_from_MQbrain_194_l | 7.00E−04 | −0.0024 |
| [54,] hsa-mir-29c_r | 3.00E−04 | −0.0012 |
| [55,] HSbrain_1384_r | 1.00E−04 | −4.00E−04 |

CLASSIFICATION COLORECTAL ADENOMA VS CONTROL
>pamr.listgenes(pamr_cgh.class.trained, pamr_cgh.class, threshold = threshold.chosen, genenames = FALSE)

TABLE 5b
Statistical analysis

prediction

| | | | |
|---|---|---|---|
| tumor type | adenoma | control | Class. Error rate |

CLASSIFICATION COLORECTAL ADENOMA VS CONTROL
>pamr.confusion(pamr_cgh.class.cv, threshold = threshold.chosen)

| | | | |
|---|---|---|---|
| adenoma | 27 | 6 | 0.181818 |
| control | 3 | 6 | 0.333333 |
| overall error rate | | | 0.212 |

AFTER REMOVING THE 55 MIRNAS
>pamr.confusion(pamr_cgh.class.cv, threshold = threshold.chosen)

| | | | |
|---|---|---|---|
| adenoma | 24 | 9 | 0.272727 |
| control | 6 | 3 | 0.666667 |
| overall error rate | | | 0.349 |

TABLE 6a
List of small RNAs as good classifiers to distinguish colorectal control
from adenocarcinoma tissue
CLASSIFICATION COLORECTAL ADENOCARCINOMA VS
CONTROL
>pamr.listgenes(pamr_cgh.class.trained, pamr_cgh.class, threshold =
threshold.chosen, genenames = FALSE)

| | carcinoma | control |
|---|---|---|
| Systematic name | score | score |
| [1,] HS_from_PT_114_r | 0.1516 | −0.5222 |
| [2,] HSbrain_377_l | 0.1495 | −0.5149 |
| [3,] hsa-mir-92-2_r | 0.1355 | −0.4668 |
| [4,] hsa-mir-92-1_r | 0.1346 | −0.4635 |
| [5,] HS_from_PT_114_l | 0.118 | −0.4063 |
| [6,] HSbrain_377_r | 0.099 | −0.341 |
| [7,] hsa-mir-145_l | 0.0883 | −0.304 |
| [8,] hsa-mir-92b_r | 0.0729 | −0.2512 |
| [9,] hsa-mir-199a-2_r | 0.0619 | −0.2134 |
| [10,] hsa-mir-29a_r | 0.0599 | −0.2064 |
| [11,] HSbrain_656_l | −0.0536 | 0.1846 |
| [12,] hsa-mir-124a-2_r | −0.0507 | 0.1746 |
| [13,] hsa-mir-29c_r | 0.0503 | −0.1734 |
| [14,] hsa-mir-124a-1_r | −0.0475 | 0.1635 |
| [15,] HS_from_PT_155_l | −0.0468 | 0.1613 |
| [16,] HSbrain_411_r | 0.0468 | −0.1612 |
| [17,] HS_from_MQbrain_991_l | −0.0443 | 0.1525 |
| [18,] HS_291_r | 0.0435 | −0.1498 |
| [19,] hsa-mir-27a_r | 0.0433 | −0.1492 |
| [20,] HS_from_MQbrain_2700_r | 0.0431 | −0.1485 |
| [21,] HS_from_MQbrain_2630_l | −0.0417 | 0.1437 |
| [22,] hsa-mir-124a-3_r | −0.0417 | 0.1435 |
| [23,] hsa-mir-635_l | −0.0413 | 0.1423 |
| [24,] hsa-let-7f-1_l | 0.0405 | −0.1396 |
| [25,] hsa-let-7a-1_l | 0.04 | −0.1377 |
| [26,] hsa-mir-199a-1_r | 0.0393 | −0.1353 |
| [27,] hsa-let-7a-2_l | 0.0384 | −0.1324 |
| [28,] HS_from_MQbrain_1792_l | −0.0384 | 0.1323 |
| [29,] hsa-let-7f-2_l | 0.0376 | −0.1295 |
| [30,] HS_from_MQbrain_1073_l | −0.0373 | 0.1285 |
| [31,] hsa-mir-564_r | −0.0367 | 0.1263 |
| [32,] hsa-mir-573_l | 0.0355 | −0.1223 |
| [33,] HS_from_MQbrain_1320_r | −0.0347 | 0.1197 |
| [34,] hsa-mir-21_l | 0.0328 | −0.113 |
| [35,] HS_from_MQbrain_594_r | −0.0311 | 0.1073 |
| [36,] hsa-mir-98_l | 0.0301 | −0.1037 |
| [37,] HSbrain_638_l | −0.0291 | 0.1001 |
| [38,] hsa-mir-20b_l | 0.0274 | −0.0945 |
| [39,] hsa-mir-768_r | 0.0269 | −0.0926 |
| [40,] HSbrain_1437_r | 0.0267 | −0.0918 |
| [41,] hsa-mir-181c_l | 0.0265 | −0.0913 |
| [42,] HS_from_MQbrain_967_l | −0.024 | 0.0826 |
| [43,] HS_291_r | 0.0236 | −0.0813 |
| [44,] hsa-let-7a-3_l | 0.0233 | −0.0803 |
| [45,] HS_from_MQbrain_855_r | −0.0231 | 0.0796 |
| [46,] hsa-mir-20a_l | 0.0219 | −0.0755 |
| [47,] HSbrain_99_l | −0.0206 | 0.0711 |
| [48,] hsa-mir-181a-1_l | 0.0203 | −0.07 |
| [49,] HS_from_PT_104_l | −0.0201 | 0.0693 |
| [50,] HSbrain_172_l | −0.0199 | 0.0686 |
| [51,] HSbrain_426_r | 0.0184 | −0.0634 |
| [52,] HSbrain_398_r | 0.0181 | −0.0623 |
| [53,] HS_from_MQbrain_340_l | −0.018 | 0.0619 |
| [54,] HSbrain_500_r | 0.0179 | −0.0616 |
| [55,] HSbrain_120_r | −0.017 | 0.0584 |
| [56,] HS_from_MQbrain_1497_l | −0.0166 | 0.0573 |
| [57,] U6_snRNA | 0.0156 | −0.0538 |
| [58,] hsa-let-7g_l | 0.0156 | −0.0538 |
| [59,] HS_from_MQbrain_1793_l | −0.0156 | 0.0536 |
| [60,] HS_from_MM_353_l | −0.0141 | 0.0486 |
| [61,] HSbrain_100_l | −0.0136 | 0.0467 |
| [62,] HS_from_MM_210_l | −0.0132 | 0.0453 |
| [63,] HSbrain_704_l | −0.0131 | 0.045 |
| [64,] HS_from_MQbrain_279_l | −0.0124 | 0.0427 |
| [65,] HS_from_MQbrain_485_l | −0.0108 | 0.0372 |
| [66,] Hsd_from_Mmd_328_l | −0.0101 | 0.0348 |
| [67,] hsa-mir-372_r | −0.0037 | 0.0128 |
| [68,] hsa-mir-368_l | −0.0034 | 0.0115 |
| [69,] hsa-mir-17_l | 0.0033 | −0.0114 |
| [70,] HS_from_MQbrain_1432_l | −0.003 | 0.0104 |
| [71,] HS_from_MM_178_l | −0.0029 | 0.0098 |
| [72,] HSbrain_499_l | −0.0028 | 0.0098 |
| [73,] hsa-mir-126_r | 0.0026 | −0.0089 |
| [74,] HS_from_PT_146_r | 0.0025 | −0.0086 |
| [75,] hsa-mir-125a_l | 0.0018 | −0.0062 |
| [76,] HS_from_MQbrain_827_l | −0.0015 | 0.0053 |
| [77,] HS_215_l | −8.00E−04 | 0.0027 |
| [78,] HS_from_MQbrain_755_l | −5.00E−04 | 0.0016 |
| [79,] HS_from_MM_47_r | −3.00E−04 | 0.001 |

TABLE 6b
Statistical analysis of classifier

prediction

| | | | |
|---|---|---|---|
| Tumor type | carcinoma | control | Class. Error rate |

CLASSIFICATION COLORECTAL ADENOCARCINOMA VS
CONTROL
>pamr.confusion(pamr_cgh.class.cv, threshold = threshold.chosen)

| | | | |
|---|---|---|---|
| carcinoma | 25 | 6 | 0.193548 |
| control | 2 | 7 | 0.222222 |
| overall error rate | | | 0.198 |

After removing the 79 miRNAs

| | | | |
|---|---|---|---|
| carcinoma | 24 | 7 | 0.225807 |
| control | 8 | 1 | 0.888889 |
| overall error rate | | | 0.365 |

TABLE 7a
List of small RNAs as good classifiers to distinguish colorectal adenoma
from adenocarcinoma tissue
CLASSIFICATION OF COLORECTAL ADENOMA VS
ADENOCARCINOMA
>pamr.listgenes(pamr_cgh.class.trained, pamr_cgh.class,
threshold = threshold.chosen, genenames = FALSE)

| Systematic name | Ad-score | Carc-score |
|---|---|---|
| [1,] HS_from_MQbrain_1082_l | −0.0915 | 0.0974 |
| [2,] HS_51_r | −0.0897 | 0.0955 |
| [3,] HS_from_MQbrain_1334_r | −0.0794 | 0.0845 |
| [4,] HS_from_MQbrain_1620_r | −0.0596 | 0.0635 |
| [5,] HS_from_MQbrain_1099_r | −0.0574 | 0.0611 |
| [6,] HS_from_MQbrain_63_r | −0.0494 | 0.0526 |
| [7,] HS_176_l | −0.0407 | 0.0433 |
| [8,] HSbrain_687_r | −0.0366 | 0.039 |
| [9,] hsa-mir-375_r | 0.0342 | −0.0364 |
| [10,] HS_from_MQbrain_458_l | −0.0301 | 0.0321 |
| [11,] HS_from_MQbrain_638_l | −0.03 | 0.032 |
| [12,] HS_from_MM_339_l | −0.0299 | 0.0318 |
| [13,] HSbrain_684_r | −0.0258 | 0.0275 |
| [14,] HSbrain_680_r | −0.0236 | 0.0252 |
| [15,] HS_136_r | −0.0233 | 0.0248 |
| [16,] HS_from_MQbrain_1204_l | −0.0196 | 0.0209 |
| [17,] hsa-mir-136_l | −0.0177 | 0.0188 |
| [18,] HS_from_PT_36_l | −0.0173 | 0.0184 |
| [19,] HSbrain_390_l | −0.0168 | 0.0179 |
| [20,] HSbrain_570_r | −0.0135 | 0.0143 |
| [21,] HSbrain_669_r | −0.0122 | 0.013 |
| [22,] HS_from_MQbrain_824_l | −0.011 | 0.0117 |
| [23,] HS_from_MQbrain_825_r | −0.0052 | 0.0056 |
| [24,] HS_from_PT_201_l | −0.0032 | 0.0034 |
| [25,] HS_from_MQbrain_1985_l | 0.0025 | −0.0027 |
| [26,] HS_from_MQbrain_579_l | 0.0022 | −0.0023 |

TABLE 7b
Statistical analysis of classifier

prediction

| | | | |
|---|---|---|---|
| tumor type | adenoma | carcinoma | Class. Error rate |

>pamr.confusion(pamr_cgh.class.cv, threshold = threshold.chosen)
CLASSIFICATION OF COLORECTAL ADENOMA VS
ADENOCARCINOMA CLASSIFICATION

| | | | |
|---|---|---|---|
| adenoma | 25 | 8 | 0.242424 |
| carcinoma | 8 | 23 | 0.258065 |
| overall error | | | 0.248 |
| rate | | | |

AFTER EXCLUDING THE 26 MIRNAS

| | | | |
|---|---|---|---|
| adenoma | 23 | 10 | 0.30303 |
| carcinoma | 15 | 16 | 0.483871 |
| overall error | | | 0.386 |
| rate | | | |

TABLE 8
Recovered small RNAs in faeces

| Systematic name | signal/background¹ |
|---|---|
| HS_from_MQbrain_1596_r | 435.4766611 |
| HS_from_MQbrain_610_r | 397.2236261 |
| HS_from_MQbrain_1365_r | 381.7698338 |
| HS_from_MQbrain_1686_l | 381.0649897 |
| HS_from_MQbrain_1748_l | 379.1642471 |
| HS_from_MQbrain_397_l | 374.0176792 |
| HS_from_MQbrain_1006_r | 373.1830201 |
| HS_from_PT_134_l | 371.7744239 |
| HSbrain_367_r | 365.4950355 |
| HS_from_MQbrain_1575_r | 363.8207777 |
| HS_from_MQbrain_1245_r | 362.2517824 |
| HSbrain_154_l | 356.5198479 |
| Hsd_from_Mmd_472_r | 353.9520892 |
| HSbrain_797_l | 352.8430904 |
| HS_from_MM_315_l | 352.564084 |
| Hsd_from_Mmd_167_r | 352.3358142 |
| HS_from_MQbrain_520_r | 352.1386318 |
| HS_148_r | 351.4766543 |
| HSbrain_104_l | 351.1481239 |
| HS_from_MM_347_l | 350.4004069 |
| HS_from_MQbrain_1053_r | 350.2064947 |
| HSbrain_402_l | 349.5145921 |
| HSbrain_750_l | 346.8737514 |
| HS_from_MM_73_l | 346.8607343 |
| HSbrain_783_l | 345.0006901 |
| HS_from_MQbrain_1185_r | 344.3922571 |
| HS_from_MQbrain_498_r | 342.07876 |
| HS_from_MQbrain_684_r | 341.5414126 |
| Hsd_from_Mmd_424_r | 340.2003175 |
| HS_64_l | 340.1621279 |
| HSbrain_316_l | 339.4367001 |
| HS_from_MQbrain_747_l | 339.3507657 |
| HS_from_MQbrain_301_r | 337.8052813 |
| HS_from_MQbrain_1833_r | 336.9120561 |
| HSbrain_393_r | 336.1272016 |
| HSbrain_761_r | 335.820625 |
| HSbrain_467_l | 335.0856427 |
| HSbrain_259_r | 334.9867442 |
| HS_from_MQbrain_429_r | 334.982389 |
| HSbrain_268_l | 334.6908402 |
| HSbrain_326_l | 334.5936127 |
| HSbrain_365_r | 333.5124629 |
| HS_from_MQbrain_1846_r | 333.0341838 |
| HS_from_MQbrain_1727_r | 332.7123109 |
| HSbrain_129_r | 331.9994753 |
| Hsd_from_Mmd_295_r | 331.1451277 |
| HS_98_r | 330.803132 |
| Hsd_from_Mmd_510_l | 330.4724501 |
| HS_from_MQbrain_1677_l | 330.4254578 |
| HS_38_l | 330.3876378 |
| HSbrain_273_r | 329.7258072 |
| HSbrain_431_l | 329.5258621 |
| HSbrain_668_l | 328.9649666 |
| HS_from_MQbrain_1349_l | 328.4193266 |
| HS_31_l | 328.1743211 |
| HSbrain_168_l | 327.7976202 |
| HS_from_MQbrain_115_r | 327.5091884 |
| HS_from_MM_339_r | 327.2871875 |
| HS_from_MM_355_l | 326.4926227 |
| HS_249_l | 326.2091382 |
| HS_from_MM_216_r | 326.1851794 |
| HS_from_MQbrain_341_l | 325.8932327 |
| HS_123_l | 325.8368333 |
| HS_from_MQbrain_544_r | 325.7380605 |
| HSbrain_869_r | 325.529766 |
| HS_from_MQbrain_731_l | 324.8256023 |
| HS_from_MM_240_r | 324.7437047 |
| HS_from_MQbrain_1339_l | 322.8532283 |
| HSbrain_105_l | 321.4268792 |
| HS_from_MQbrain_1711_r | 320.9572229 |
| HS_from_MQbrain_1652_r | 320.4545063 |
| HS_from_MQbrain_1470_r | 320.2896415 |
| Hsd_66_r | 320.1767206 |
| HS_39_r | 320.0335858 |
| HS_from_MQbrain_1073_r | 319.8894959 |
| HS_156_r | 319.2763913 |
| HS_from_MQbrain_1376_l | 319.0687179 |
| Hsd_39_l | 318.8240319 |
| HSbrain_490_r | 318.789703 |
| HS_from_MQbrain_753_l | 318.6834937 |
| HS_from_MQbrain_928_r | 318.1021923 |
| HS_from_MQbrain_1210_l | 318.0464645 |
| HSbrain_202_r | 317.8857843 |
| HS_from_MM_351_r | 317.8521227 |
| HSbrain_323_l | 317.2801386 |
| HS_from_PT_101_l | 317.1749629 |
| HS_from_MQbrain_559_l | 316.9112294 |
| HS_from_MQbrain_1119_l | 316.6560497 |
| HS_from_PT_185_r | 316.2367142 |
| HS_from_MQbrain_491_l | 315.5363383 |
| HSbrain_218_r | 315.5273574 |
| HS_from_MQbrain_162_r | 314.656125 |
| HSbrain_802_l | 314.4005359 |
| HS_222_r | 313.9881647 |
| HSbrain_673_l | 313.971615 |
| HS_from_MQbrain_158_r | 313.8141203 |
| HS_from_MQbrain_76_l | 313.7393384 |
| HSbrain_250_l | 313.5276305 |
| HS_from_MQbrain_33_l | 313.4922139 |
| HS_from_MQbrain_1237_l | 313.2573889 |
| HS_25_r | 313.2035431 |
| HS_from_PT_226_r | 313.0722924 |
| HS_from_MQbrain_1018_l | 312.5603857 |
| HS_from_MQbrain_1306_r | 312.3615258 |
| HSbrain_93_l | 312.1999205 |
| HSbrain_118_r | 311.3195051 |
| HSbrain_355_r | 310.7457981 |
| HSbrain_731_l | 310.1586585 |
| HS_from_MQbrain_886_r | 310.0903943 |
| HS_from_PT_231_r | 310 |
| HSbrain_1413_r | 309.9504964 |
| HS_from_MQbrain_1355_r | 309.6691714 |
| HS_279_l | 309.5850497 |
| Hsd_from_Mmd_246_r | 309.4213909 |
| Hsd_from_Mmd_344_r | 309.0726717 |
| HS_from_MQbrain_1797_r | 308.3812108 |
| HS_263_r | 308.2617369 |
| HS_from_MQbrain_495_l | 307.6030143 |
| HS_from_MQbrain_1475_l | 307.3970336 |
| HS_from_MQbrain_1890_r | 307.317464 |
| HS_from_MQbrain_1697_r | 307.3045602 |
| HS_from_MQbrain_1620_l | 307.2296305 |
| HSbrain_669_l | 307.2172689 |
| HS_from_PT_55_l | 307.1963218 |
| HS_206_l | 307.0627996 |
| Hsd_from_Mmd_237_l | 306.9716516 |
| HS_265_r | 306.7532262 |
| HS_from_MM_324_l | 306.7520389 |
| HS_from_MQbrain_1701_l | 306.7083454 |
| HSbrain_166_l | 305.2218421 |
| Hsd_from_Mmd_56_r | 305.1038545 |
| HS_55_l | 304.7252967 |
| HS_179_l | 304.5575276 |
| HS_from_MQbrain_1102_l | 303.4601583 |
| HS_from_PT_92_l | 303.3056853 |
| HS_from_MQbrain_996_l | 302.9472283 |
| HSbrain_47_l | 302.9456152 |
| HS_from_MQbrain_1134_l | 302.6041466 |
| HSbrain_739_l | 302.4299797 |
| HS_from_MQbrain_1504_r | 302.2117858 |
| HSbrain_728_r | 302.1421754 |
| HS_from_MQbrain_1549_r | 302.1121702 |
| HS_from_PT_305_l | 301.2719608 |
| HS_from_MQbrain_1219_l | 301.2674923 |
| HS_from_PT_89_l | 301.0441042 |
| HS_from_MQbrain_1313_l | 300.2100934 |
| HS_197_r | 299.8398179 |
| HS_139_l | 299.5699263 |
| HS_from_MQbrain_1412_r | 299.4327293 |
| HS_from_MQbrain_114_r | 298.9587162 |
| HSbrain_244_r | 298.9169907 |
| HS_from_MQbrain_824_r | 298.2022039 |
| HSbrain_293_r | 298.0504476 |
| HSbrain_279_r | 297.4930175 |
| Hsd_50_l | 297.3692697 |
| HSbrain_508_r | 297.2569514 |
| Hsd_from_Mmd_405_r | 296.9247748 |
| HSbrain_39_r | 296.7631653 |
| HS_233_r | 296.5405897 |
| HSbrain_1247_r | 296.4550519 |
| Hsd_from_Mmd_28_r | 296.3415549 |
| HSbrain_299_l | 295.6857933 |
| HS_86_l | 295.4181206 |
| HS_from_MQbrain_1876_r | 294.6889637 |
| HS_from_MQbrain_1605_r | 294.5310073 |
| HS_19_l | 294.2259027 |
| HS_from_MQbrain_1367_r | 293.8593208 |
| HS_212_r | 293.7948013 |
| HS_from_MQbrain_198_l | 293.4862508 |
| HS_from_MQbrain_1446_r | 293.2223329 |
| HSbrain_410_r | 293.1926573 |
| HS_194_l | 292.9822677 |
| HS_from_MQbrain_1798_r | 292.9376997 |
| Hsd_from_Mmd_198_r | 292.8517224 |
| HS_from_MQbrain_280_r | 292.4992309 |
| HS_from_MM_200_r | 292.4872858 |
| HS_from_MQbrain_1824_l | 292.4713609 |
| HS_from_MQbrain_863_r | 292.4083724 |
| HS_from_MQbrain_1344_l | 292.3514496 |
| Hsd_70_l | 292.1907862 |
| HS_171_l | 291.7377757 |
| HS_from_MQbrain_1390_l | 291.6054286 |
| HSbrain_103_l | 291.3575433 |
| HS_from_MQbrain_1845_r | 291.144748 |
| HS_from_MQbrain_566_r | 291.1249796 |
| HS_from_MQbrain_1083_l | 290.7735015 |
| HSbrain_550_r | 290.7409535 |
| HS_from_MQbrain_93_r | 290.7380082 |
| HS_from_MQbrain_1838_l | 290.6155727 |
| HS_from_MQbrain_211_l | 290.3053197 |
| HSbrain_92_l | 290.3024103 |
| HS_from_MQbrain_604_l | 289.730194 |
| HS_from_MQbrain_1827_l | 289.495665 |
| HS_from_MM_239_l | 289.3328779 |
| HSbrain_549_l | 289.1694754 |
| HS_from_MQbrain_1392_r | 288.9210201 |
| HS_from_PT_237_r | 288.8596386 |
| HS_from_MQbrain_546_l | 288.7897335 |
| HS_from_MQbrain_850_l | 288.5662281 |
| HS_from_MQbrain_1675_r | 288.3970715 |
| HS_from_MQbrain_1601_r | 288.3631003 |
| HS_129_l | 287.8783772 |
| HS_from_MQbrain_183_l | 287.7450634 |
| HS_from_MQbrain_574_l | 287.7017103 |
| HSbrain_48_l | 287.6708455 |
| HS_from_MQbrain_1761_l | 287.6145743 |
| HS_from_PT_28_l | 287.5647925 |
| HS_from_MQbrain_218_l | 287.5544126 |
| HSbrain_679_l | 287.4730955 |
| Hsd_from_Mmd_98_r | 286.665116 |
| HSbrain_692_r | 285.9738106 |
| HS_24_r | 285.9042671 |
| Hsd_38_r | 285.7326061 |
| HS_from_MQbrain_1780_r | 285.521158 |
| HS_from_MM_289_r | 285.4582707 |
| HS_221_r | 285.3734928 |
| HS_from_MQbrain_70_l | 285.3653998 |
| HS_154_l | 285.3397575 |
| HSbrain_192_l | 285.0443884 |
| HS_from_MQbrain_1683_l | 285.0291226 |
| HSbrain_290_l | 284.9517611 |
| HSbrain_109_l | 284.6354613 |
| HSbrain_628_r | 284.241639 |
| HS_from_MQbrain_1537_l | 283.6533255 |
| HSbrain_1425_l | 283.5640729 |
| HS_from_MM_202_r | 283.5284034 |
| HS_from_MQbrain_1509_r | 283.4583708 |
| HSbrain_390_r | 283.2625881 |
| HSbrain_44_l | 283.2059478 |
| HS_from_MQbrain_1307_l | 283.1729339 |
| HS_from_MQbrain_1862_r | 283.0420705 |
| HS_from_MQbrain_487_r | 282.9624273 |
| HS_from_MQbrain_1272_l | 282.8549789 |
| HSbrain_584_r | 282.7973454 |
| HS_from_MQbrain_1458_r | 282.7887074 |
| HS_from_MQbrain_267_l | 282.6850078 |
| HS_from_MQbrain_303_l | 282.3294611 |
| Hsd_1_l | 281.9403791 |
| HS_from_MQbrain_375_r | 281.6424907 |
| HSbrain_372_l | 281.6246493 |
| HS_from_MQbrain_1043_r | 281.5762963 |
| HSbrain_311_r | 281.0626983 |
| HSbrain_404_r | 280.8722291 |
| HSbrain_328_l | 280.7719926 |
| HS_from_MQbrain_917_r | 280.5140354 |
| Hsd_from_Mmd_483_l | 280.4528223 |
| HS_from_MQbrain_655_r | 280.3486118 |
| HS_from_MQbrain_580_l | 280.2794277 |
| HS_from_MQbrain_1720_l | 280.052876 |
| HSbrain_35_r | 279.8399868 |
| HS_from_PT_43_l | 279.7988575 |
| HS_from_MQbrain_461_r | 279.2472579 |
| HS_from_MQbrain_2700_l | 279.1948493 |
| HS_134_r | 278.9094666 |
| HSbrain_637_l | 278.8901829 |
| HS_188_r | 278.645448 |
| HS_from_MQbrain_1747_r | 278.4365165 |
| HS_from_MQbrain_1635_r | 278.3933949 |
| HS_from_MQbrain_333_r | 278.3594605 |
| HS_from_PT_94_r | 278.3423878 |
| Hsd_from_Mmd_480_r | 278.0451339 |
| HS_from_MQbrain_1822_r | 277.9643504 |
| HSbrain_141_r | 277.8002235 |
| HS_from_MQbrain_436_l | 277.7371995 |
| HS_from_MQbrain_3_r | 277.7328797 |
| HSbrain_101_l | 277.3680398 |
| HS_from_MQbrain_1394_l | 277.0531964 |
| HS_from_MQbrain_496_r | 276.9628577 |
| HS_from_PT_286_l | 276.7057792 |
| HSbrain_729_l | 276.5800842 |
| HS_from_MQbrain_913_r | 276.4621645 |
| HS_from_MM_117_r | 276.4579506 |
| HS_from_MM_195_r | 276.4162313 |
| HSbrain_252_r | 276.2314589 |
| HS_from_MQbrain_751_r | 276.1913135 |
| HSbrain_778_l | 276.1268612 |
| HS_284_l | 275.9294255 |
| HS_21_l | 275.8945186 |
| HS_from_MQbrain_737_l | 275.8865362 |
| HSbrain_626_l | 275.7891397 |
| HS_from_MQbrain_1832_l | 275.5897539 |
| HS_from_MQbrain_709_r | 275.3155471 |
| HSbrain_1426_l | 275.2703704 |
| HS_from_MQbrain_893_l | 274.9993461 |
| HSbrain_810_l | 274.8805235 |
| HS_from_MQbrain_736_l | 274.8508496 |
| HSbrain_709_l | 274.820351 |
| Hsd_from_Mmd_258_l | 274.7024515 |
| Hsd_from_Mmd_517_l | 274.4801 |
| HS_from_MQbrain_152_r | 274.3078669 |
| HSbrain_534_l | 274.1093388 |
| Hsd_74_r | 274.0203487 |
| HS_7_r | 273.8420324 |
| HSbrain_607_l | 273.6928046 |
| HSbrain_469_r | 273.5380323 |
| HS_122_l | 273.512663 |
| HS_40_l | 273.499248 |
| Hsd_69_l | 273.3125807 |
| HSbrain_932_r | 273.2005064 |
| HS_from_PT_262_r | 273.0777115 |
| HS_124_l | 273.022187 |
| HSbrain_221_l | 273.0060427 |
| HS_from_MQbrain_1816_r | 272.9813113 |
| HS_50_l | 272.9010802 |
| HS_from_MQbrain_721_r | 272.748855 |
| HSbrain_745_r | 272.6274939 |
| HS_from_MQbrain_734_l | 272.5872546 |
| HS_248_l | 272.4865373 |
| HS_from_MQbrain_1523_r | 272.3792039 |
| HS_from_MQbrain_1867_l | 272.3572417 |
| HS_from_MQbrain_745_r | 271.8779897 |
| Hsd_100_l | 271.6852745 |
| HS_from_MQbrain_1777_l | 271.5768067 |
| HSbrain_266_l | 271.4320379 |
| HSbrain_1424_l | 271.024652 |
| HS_from_MQbrain_1812_r | 270.7376586 |
| HS_from_MQbrain_1421_l | 270.6118637 |
| HS_from_MQbrain_1295_r | 270.4897378 |
| HS_from_MQbrain_1297_l | 270.3967365 |
| HS_from_MQbrain_106_l | 270.3609488 |
| HS_from_MQbrain_325_l | 270.247673 |
| HS_from_MQbrain_111_r | 270.1965492 |
| HSbrain_484_l | 270.1785451 |
| HS_from_MQbrain_102_r | 270.159337 |
| HS_from_PT_44_l | 269.9446191 |
| HS_from_MQbrain_620_l | 269.779924 |
| HSbrain_588_l | 269.5888825 |
| HS_from_MQbrain_1463_l | 269.5851659 |
| HS_from_MQbrain_18_l | 269.5064948 |
| HS_from_MQbrain_688_l | 269.4728687 |
| HS_from_MQbrain_387_l | 269.3705363 |
| HS_from_MQbrain_1633_l | 269.2334364 |
| HSbrain_536_r | 269.1404787 |
| HS_from_MQbrain_738_l | 269.1111724 |
| HSbrain_78_l | 268.820699 |
| HS_from_MQbrain_1416_l | 268.5766334 |
| HS_from_MQbrain_540_l | 268.4654883 |
| Hsd_from_Mmd_141_l | 268.0968571 |
| HSbrain_511_r | 267.9991684 |
| HS_from_MQbrain_1159_l | 267.9987738 |
| HSbrain_145_l | 267.9664391 |
| HS_from_MQbrain_1755_l | 267.9405008 |
| HSbrain_632_r | 267.8498087 |
| HS_from_MQbrain_605_r | 267.8150613 |
| HS_from_MQbrain_698_l | 267.651359 |
| HSbrain_43_l | 267.5817178 |
| HS_from_MQbrain_1085_r | 267.5466978 |
| HSbrain_283_l | 267.5118039 |
| HS_from_MQbrain_1655_l | 267.3276129 |
| HS_from_MQbrain_576_l | 267.323134 |
| HS_from_MQbrain_877_l | 267.1360371 |
| HS_from_MQbrain_914_l | 266.9248651 |
| HSbrain_581_r | 266.8695989 |
| HSbrain_108_l | 266.7753944 |
| HS_from_MQbrain_933_l | 266.6934889 |
| Hsd_from_Mmd_42_r | 266.6423152 |
| HS_111_r | 266.2298017 |
| Hsd_from_Mmd_291_r | 266.1652183 |
| HSbrain_794_l | 265.9452768 |
| HS_from_MQbrain_1164_r | 265.941125 |
| HS_from_PT_300_l | 265.6728802 |
| HSbrain_241_l | 265.4661947 |
| HS_from_MQbrain_284_r | 265.4059563 |
| HS_from_PT_26_l | 265.3480358 |
| HSbrain_160_l | 265.1440349 |
| HS_from_MQbrain_1082_l | 265.0804535 |
| HSbrain_1325_l | 265.0318212 |
| HS_from_MQbrain_1587_r | 264.917603 |
| HS_234_r | 264.9059299 |
| HS_from_MQbrain_6_r | 264.8578117 |
| HS_from_MM_295_r | 264.8300478 |
| HS_274_l | 264.760369 |
| HS_from_MQbrain_2669_r | 264.5475927 |
| HS_from_MM_215_l | 264.546988 |
| HSbrain_85_l | 264.5381922 |
| HS_from_MQbrain_308_l | 264.4473766 |
| HS_from_MM_234_r | 264.3415274 |
| HSbrain_309_r | 264.2249057 |
| Hsd_from_Mmd_299_l | 264.045778 |
| HSbrain_1336_r | 263.9717575 |
| HS_from_MQbrain_1031_r | 263.8971038 |
| HS_from_MM_153_r | 263.8347068 |
| HSbrain_167_l | 263.8194016 |
| HS_from_MQbrain_671_r | 263.6821505 |
| HS_from_MQbrain_409_l | 263.461086 |
| HS_from_MQbrain_1897_r | 263.4341793 |
| HSbrain_744_r | 263.2337823 |
| HS_230_r | 262.8565928 |
| HS_from_MQbrain_1860_l | 262.7008017 |
| Hsd_from_Mmd_529_l | 262.6611279 |
| HSbrain_161_r | 262.2125657 |
| HSbrain_426_r | 262.208223 |
| HS_from_MQbrain_1889_l | 262.029435 |
| HSbrain_611_l | 261.8840849 |
| HSbrain_687_l | 261.7764626 |
| HS_from_MQbrain_1581_l | 261.626211 |
| HS_from_PT_3_r | 261.2984224 |
| HS_from_MQbrain_830_l | 261.2050408 |
| HS_from_MQbrain_782_r | 261.0759728 |
| HS_from_MQbrain_947_r | 261.0750459 |
| HSbrain_152_l | 261.0331392 |
| HS_from_MQbrain_867_r | 260.9771533 |
| HS_from_MQbrain_238_l | 260.9583211 |
| HSbrain_315_l | 260.8145615 |
| HS_from_MQbrain_458_r | 260.7547184 |
| HS_from_MQbrain_1007_l | 260.6484254 |
| HSbrain_441_l | 260.6089658 |
| HS_from_MQbrain_1709_r | 260.5526526 |
| HS_from_MQbrain_868_l | 260.4645119 |
| Hsd_42_r | 260.2142458 |
| HS_89_l | 259.9744451 |
| HS_from_MQbrain_686_r | 259.8101317 |
| Hsd_56_l | 259.7972361 |
| HS_from_MQbrain_1616_l | 259.7668701 |
| HS_from_MQbrain_1236_l | 259.6868468 |
| HSbrain_688_l | 259.6527506 |
| HSbrain_551_l | 259.5465988 |
| Hsd_from_Mmd_487_l | 259.5343392 |
| HS_from_PT_214_r | 259.3539306 |
| HSbrain_554_l | 259.3095925 |
| HS_from_MQbrain_1715_l | 259.1751881 |
| HSbrain_718_l | 259.1468547 |
| HS_from_MQbrain_999_l | 259.1426385 |
| HS_from_PT_34_r | 259.0811135 |
| HS_from_MQbrain_960_l | 258.9661306 |
| HS_from_MQbrain_1075_l | 258.9475578 |
| HS_from_MQbrain_1299_r | 258.8627877 |
| HS_from_MM_83_r | 258.6901737 |
| HS_from_MQbrain_1874_r | 258.5072003 |
| HSbrain_485_r | 258.4877461 |
| HS_5_l | 258.4229952 |
| HS_from_MQbrain_927_l | 258.383763 |
| HS_from_MQbrain_1743_r | 258.3823305 |
| HS_87_r | 258.2193761 |
| HS_from_MQbrain_477_l | 258.0217594 |
| HS_from_MQbrain_920_r | 257.858748 |
| HS_291_l | 257.3504875 |
| HSbrain_776_r | 257.2698457 |
| HS_from_MQbrain_925_l | 257.239961 |
| HS_from_MQbrain_741_r | 257.2034813 |
| HS_from_MQbrain_1459_l | 257.1079597 |
| HSbrain_284_l | 257.0803228 |
| HS_from_MQbrain_977_r | 256.7795957 |
| HSbrain_1262_l | 256.5894318 |
| HS_from_MQbrain_413_r | 256.5290041 |
| HS_from_MQbrain_1500_r | 256.5209467 |
| HSbrain_1377_r | 256.4853012 |
| HS_from_MQbrain_184_r | 256.4572439 |
| HS_from_MQbrain_1399_l | 256.4469129 |
| HSbrain_115_r | 256.2784798 |
| Hsd_from_Mmd_120_r | 256.1765933 |
| HSbrain_303_l | 256.101294 |
| HSbrain_684_l | 255.9377011 |
| HS_115_l | 255.8640176 |
| HS_from_MQbrain_1699_l | 255.8617681 |
| Hsd_17_l | 255.7570196 |
| HSbrain_614_l | 255.7168248 |
| HSbrain_171_l | 255.6856718 |
| HS_from_MQbrain_67_l | 255.5891945 |
| HS_from_MQbrain_531_r | 255.5303715 |
| HS_28_r | 255.3509233 |
| HS_from_MQbrain_11_r | 255.1937305 |
| HS_from_MM_111_r | 254.8753709 |
| HS_from_MQbrain_392_l | 254.8014108 |
| HS_from_MQbrain_1664_r | 254.7961518 |
| HSbrain_371_l | 254.7715809 |
| HS_from_MM_293_r | 254.6801321 |
| HS_219_l | 254.6760586 |
| HS_from_MQbrain_765_l | 254.6466837 |
| HS_from_MQbrain_716_r | 254.6415782 |
| HS_286_l | 254.4260526 |
| HS_from_MQbrain_475_r | 254.3932773 |
| HSbrain_151_l | 254.3505933 |
| HSbrain_629_l | 254.3371274 |
| HS_from_PT_235_r | 254.0382243 |
| HS_from_MQbrain_52_r | 254.0379479 |
| HSbrain_240_l | 253.8771621 |
| HS_from_MQbrain_181_r | 253.818416 |
| HS_199_r | 253.7948113 |
| HS_167_r | 253.7791765 |
| HS_from_MQbrain_550_l | 253.7757692 |
| HSbrain_95_l | 253.768018 |
| HSbrain_631_r | 253.7613205 |
| HS_from_MQbrain_1380_r | 253.5211282 |
| HS_149_l | 253.504048 |
| HS_from_MQbrain_1263_l | 253.4875034 |
| Hsd_from_Mmd_128_r | 253.41348 |
| HS_from_MQbrain_660_r | 253.3359551 |
| HS_from_MQbrain_524_r | 253.3349168 |
| Hsd_from_Mmd_535_l | 253.3218789 |
| HS_75_l | 253.2833704 |
| HS_from_MQbrain_1808_r | 253.1879056 |
| HS_from_PT_219_l | 253.1829244 |
| HS_from_MQbrain_861_l | 252.7877399 |
| Hsd_from_Mmd_303_r | 252.716811 |
| HS_from_MQbrain_1357_l | 252.6330054 |
| HS_from_MQbrain_71_r | 252.4897985 |
| HS_from_MQbrain_1756_l | 252.4873393 |
| HS_from_MQbrain_1758_r | 252.4287529 |
| HS_from_MQbrain_1725_r | 252.3188281 |
| HSbrain_207_r | 252.2600469 |
| HS_from_MQbrain_345_r | 252.2124247 |
| HS_from_MQbrain_163_r | 252.0716742 |
| HS_from_MM_318_l | 252.0703286 |
| HS_from_MQbrain_924_l | 252.0474973 |
| HSbrain_10_l | 251.9975319 |
| HSbrain_1364_l | 251.9113433 |
| HS_from_PT_105_l | 251.9105921 |
| Hsd_from_Mmd_97_r | 251.8796136 |
| HSbrain_493_l | 251.8121263 |
| HSbrain_1421_r | 251.7829815 |
| Hsd_from_Mmd_305_l | 251.5409922 |
| HSbrain_425_r | 251.4819635 |
| HS_from_MQbrain_1627_l | 251.4373669 |
| HS_from_PT_238_l | 251.4029293 |
| HS_238_r | 251.3418893 |
| HS_from_MQbrain_801_l | 251.3117452 |
| Hsd_from_Mmd_47_r | 251.253006 |
| Hsd_from_Mmd_204_l | 251.2502954 |
| HSbrain_815_r | 251.1910852 |
| HS_from_MQbrain_362_l | 251.1148036 |
| Hsd_109_l | 251.0897866 |
| HS_from_MM_121_r | 251.0682642 |
| HSbrain_203_l | 250.9770175 |
| HS_157_l | 250.822208 |
| HSbrain_522_l | 250.7517293 |
| HS_from_MQbrain_541_r | 250.6783764 |
| HSbrain_49_r | 250.6720929 |
| HS_from_MQbrain_2679_r | 250.6148031 |
| HS_from_MQbrain_38_l | 250.553482 |
| Hsd_from_Mmd_448_r | 250.5484342 |
| HS_32_l | 250.4202101 |
| HS_57_l | 250.390488 |
| HS_from_MQbrain_599_l | 250.2861907 |
| HSbrain_257_l | 250.1547679 |
| HS_from_MQbrain_672_l | 249.9938404 |
| HS_from_MQbrain_1021_l | 249.9528181 |
| HS_from_MQbrain_1202_l | 249.8836584 |
| HSbrain_693_l | 249.866378 |
| HSbrain_191_l | 249.7564415 |
| HS_from_MQbrain_1729_r | 249.7095874 |
| Hsd_34_l | 249.7070542 |
| HSbrain_319_r | 249.6719621 |
| HS_from_MQbrain_1224_l | 249.5971643 |
| HSbrain_788_l | 249.4670383 |
| HS_224_r | 249.4599231 |
| HSbrain_1298_l | 249.2142487 |
| HSbrain_800_l | 249.0877273 |
| HS_105_l | 249.0808996 |
| HS_48_l | 248.6727669 |
| HS_from_PT_102_l | 248.6580593 |
| Hsd_from_Mmd_60_r | 248.5708006 |
| HS_from_MQbrain_744_l | 248.4293356 |
| HSbrain_287_l | 248.3731626 |
| HSbrain_366_l | 248.3201407 |
| HSbrain_559_l | 248.2908649 |
| HS_from_MQbrain_1225_l | 248.1163903 |
| HS_from_PT_115_l | 248.0256549 |
| HS_from_MQbrain_974_l | 247.9593335 |
| HS_from_MQbrain_1771_r | 247.8706875 |
| HS_from_MQbrain_1211_l | 247.8586125 |
| HS_from_MQbrain_630_l | 247.7802526 |
| HS_from_MM_336_r | 247.7696355 |
| HS_from_MM_340_l | 247.7496711 |
| HS_from_MQbrain_1752_r | 247.6292267 |
| HS_from_MQbrain_338_r | 247.601172 |
| HS_from_MQbrain_36_r | 247.5789144 |
| HS_from_MQbrain_866_l | 247.563347 |
| HS_108_r | 247.5356016 |
| HS_from_MQbrain_1830_r | 247.4938017 |
| HS_from_MQbrain_149_l | 247.4259409 |
| HSbrain_703_l | 247.3420423 |
| HS_268_r | 247.3259637 |
| HSbrain_53_l | 247.0890399 |
| HS_from_MM_103_l | 246.9217731 |
| HS_26_l | 246.8184302 |
| HS_from_MQbrain_785_l | 246.6323107 |
| HS_from_MQbrain_1152_l | 246.4764836 |
| HS_from_MQbrain_331_l | 246.4586884 |
| HS_from_MQbrain_1673_r | 246.4248334 |
| HS_163_l | 246.3435123 |
| HS_from_MQbrain_155_r | 246.3195924 |
| HSbrain_428_l | 246.1892116 |
| HSbrain_1360_l | 246.1741425 |
| HSbrain_701_l | 245.9313661 |
| HS_from_MQbrain_129_l | 245.6975055 |
| HSbrain_424_l | 245.6897519 |
| HS_from_MQbrain_1630_l | 245.6614642 |
| HSbrain_680_l | 245.548398 |
| HS_from_MQbrain_560_l | 245.4991032 |
| HS_from_MQbrain_862_l | 245.4814067 |
| HS_from_MM_175_l | 245.4785201 |
| HS_from_MQbrain_229_r | 245.4629526 |
| HS_190_l | 245.2188679 |
| HS_from_PT_285_l | 244.8939459 |
| HSbrain_6_r | 244.8860331 |
| Hsd_36_r | 244.879207 |
| HS_from_PT_280_r | 244.6775996 |
| HS_from_MQbrain_852_r | 244.6680722 |
| HS_18_l | 244.6303758 |
| HS_from_MQbrain_318_l | 244.3815927 |
| HS_from_MM_115_l | 244.343368 |
| HS_from_MQbrain_1804_l | 244.2239598 |
| Hsd_from_Mmd_399_l | 244.1887779 |
| HSbrain_456_l | 244.1455475 |
| HS_from_MQbrain_1623_r | 244.0389624 |
| HS_304_r | 244.034349 |
| HS_from_MQbrain_1261_r | 243.9532889 |
| HS_from_MQbrain_1227_l | 243.816199 |
| Hsd_from_Mmd_52_r | 243.7569251 |
| HSbrain_1414_l | 243.7148738 |
| HSbrain_334_l | 243.7049445 |
| HS_200_l | 243.6162631 |
| HS_56_r | 243.5447343 |
| HS_from_MQbrain_266_l | 243.4504792 |
| HS_from_MQbrain_340_r | 243.3848161 |
| HS_from_MQbrain_42_r | 243.2240866 |
| HSbrain_37_l | 243.2175482 |
| HS_from_MQbrain_142_l | 243.1899123 |
| Hsd_from_Mmd_501_r | 243.1733615 |
| HS_from_MQbrain_697_r | 242.9519103 |
| HS_from_MQbrain_48_l | 242.7495036 |
| HS_from_PT_178_l | 242.7023061 |
| HS_from_MQbrain_1131_l | 242.6558541 |
| HS_from_MQbrain_59_r | 242.5582379 |
| HSbrain_759_r | 242.5194837 |
| HS_from_MQbrain_1426_l | 242.4325995 |
| HSbrain_685_l | 242.3863748 |
| HSbrain_387_r | 242.2536111 |
| HS_236_r | 242.1625453 |
| Hsd_from_Mmd_412_l | 242.0650219 |
| HS_from_MQbrain_1501_r | 242.0224401 |
| HS_from_MM_208_r | 241.9491489 |
| HS_from_MQbrain_562_l | 241.9126377 |
| Hsd_from_Mmd_477_r | 241.8020501 |
| HS_from_MQbrain_342_r | 241.7880108 |
| HS_from_MQbrain_1803_l | 241.674899 |
| HSbrain_107_l | 241.6141397 |
| HSbrain_717_l | 241.5872727 |
| HSbrain_291_l | 241.3015643 |
| HS_from_MQbrain_482_r | 241.2886123 |
| Hsd_12_l | 241.2816211 |
| HS_from_PT_60_l | 241.2340994 |
| HS_from_MQbrain_770_l | 241.0771247 |
| HS_from_MQbrain_543_l | 240.9192683 |
| HS_from_MQbrain_636_l | 240.8802238 |
| HS_from_MQbrain_1417_r | 240.8593014 |
| HS_from_MQbrain_902_r | 240.8313662 |
| Hsd_from_Mmd_44_l | 240.8110271 |
| HS_from_MQbrain_123_r | 240.7866904 |
| HS_from_MQbrain_739_r | 240.7727305 |
| HS_from_MM_87_l | 240.7726111 |
| HSbrain_652_l | 240.7492888 |
| HSbrain_796_l | 240.5926111 |
| Hsd_from_Mmd_277_r | 240.442969 |
| HS_6_l | 240.4257169 |
| HS_from_MQbrain_82_r | 240.3655142 |
| HS_from_MQbrain_766_l | 240.1728548 |
| HS_from_MM_45_l | 240.1478843 |
| HSbrain_621_l | 240.1453763 |
| HS_from_MQbrain_534_r | 240.0925796 |
| HS_from_MQbrain_146_r | 240.0908888 |
| HS_195_r | 240.0699517 |
| HSbrain_439_r | 240.0466077 |
| HS_65_l | 240.0227021 |
| HS_from_MQbrain_412_r | 239.9320244 |
| HS_from_MQbrain_590_r | 239.9048346 |
| HS_198_l | 239.6726 |
| HSbrain_41_l | 239.6624516 |
| Hsd_from_Mmd_185_l | 239.5086227 |
| HS_from_MQbrain_940_r | 239.4870149 |
| HS_from_MQbrain_635_r | 239.4679434 |
| Hsd_from_Mmd_112_l | 239.465298 |
| HS_from_MQbrain_257_l | 239.4468171 |
| Hsd_from_Mmd_266_r | 239.3412808 |
| HS_from_MQbrain_789_l | 239.2174834 |
| HS_from_MQbrain_1028_l | 239.0895004 |
| HS_101_l | 239.0332153 |
| HS_from_MQbrain_941_l | 238.8667114 |
| HS_from_MQbrain_1781_r | 238.7683976 |
| HS_from_PT_158_l | 238.7177237 |
| HS_from_MQbrain_1163_l | 238.6802874 |
| HS_from_PT_210_r | 238.5965 |
| HS_from_MQbrain_642_r | 238.5808146 |
| Hsd_from_Mmd_78_l | 238.3908975 |
| HSbrain_569_l | 238.3876463 |
| HSbrain_219_r | 238.381585 |
| HS_58_l | 238.3731335 |
| HS_from_MQbrain_336_l | 238.3456325 |
| HS_from_MQbrain_399_l | 238.3085443 |
| HS_from_MM_227_l | 238.2555243 |
| HS_from_MQbrain_270_l | 238.2332356 |
| HS_from_MQbrain_1855_l | 238.2044165 |
| HS_from_MQbrain_1706_r | 238.0686643 |
| Hsd_from_Mmd_518_r | 238.0578899 |
| HS_from_MQbrain_1625_r | 238.0330045 |
| HSbrain_363_r | 237.9427646 |
| HSbrain_98_l | 237.5428289 |
| Hsd_18_l | 237.5322474 |
| HS_from_MQbrain_398_r | 237.4649699 |
| HS_from_MQbrain_1754_l | 237.3450153 |
| Hsd_103_r | 237.3131276 |
| HS_from_MQbrain_1167_l | 237.3112369 |
| HS_from_MM_67_r | 237.2897545 |
| HSbrain_941_l | 237.1641025 |
| HS_from_MQbrain_723_r | 237.156234 |
| HS_from_MQbrain_750_r | 237.0499841 |
| HSbrain_134_l | 237.0267014 |
| HS_from_MQbrain_884_r | 236.9294285 |
| Hsd_from_Mmd_301_l | 236.8337487 |
| Hsd_from_Mmd_511_l | 236.8080344 |
| HSbrain_292_l | 236.7032475 |
| Hsd_from_Mmd_186_l | 236.6576528 |
| HS_from_MQbrain_108_l | 236.6551839 |
| HS_from_MQbrain_330_r | 236.6286556 |
| Hsd_from_Mmd_270_l | 236.4570545 |
| HS_from_MQbrain_1574_l | 236.4214286 |
| HS_289_l | 236.2167396 |
| HSbrain_520_l | 236.1868036 |
| HS_from_MQbrain_1681_l | 236.084045 |
| HSbrain_723_l | 236.0439796 |
| HS_from_MQbrain_1420_r | 235.9747391 |
| HS_from_MM_102_l | 235.9016922 |
| HS_218_r | 235.8613357 |
| HS_from_PT_91_l | 235.8215625 |
| HSbrain_702_l | 235.7253395 |
| HS_168_r | 235.6020265 |
| HSbrain_821_r | 235.4984573 |
| HS_from_MQbrain_965_l | 235.4627068 |
| HS_from_MQbrain_1813_l | 235.1182093 |
| HS_72_r | 235.0075281 |
| HSbrain_88_r | 234.9973414 |
| HS_from_MQbrain_447_l | 234.9547185 |
| HSbrain_784_r | 234.6735882 |
| HS_from_MQbrain_582_r | 234.546672 |
| HSbrain_66_r | 234.5308069 |
| HSbrain_361_l | 234.5281831 |
| HS_from_MQbrain_360_r | 234.4085303 |
| HS_from_MQbrain_1632_l | 234.3869839 |
| HS_from_MQbrain_1843_r | 234.2751382 |
| HSbrain_317_l | 234.263649 |
| HS_from_MQbrain_539_r | 234.1938897 |
| HSbrain_460_l | 234.1480162 |
| HSbrain_518_l | 234.0304825 |
| HS_from_MQbrain_1865_r | 233.9908466 |
| HS_246_l | 233.9810458 |
| HS_119_l | 233.7784735 |
| HS_285_r | 233.586665 |
| HS_from_MQbrain_1331_r | 233.5757256 |
| HS_from_MQbrain_89_r | 233.5080048 |
| HS_from_MQbrain_1672_r | 233.4867676 |
| HS_from_MQbrain_807_r | 233.4564439 |
| Hsd_from_Mmd_215_l | 233.3659358 |
| HS_from_MQbrain_889_r | 233.1847619 |
| HSbrain_1408_r | 233.1511963 |
| HS_from_MQbrain_404_l | 233.1396668 |
| Hsd_107_r | 233.060561 |
| HS_from_MQbrain_121_r | 232.9199135 |
| Hsd_21_l | 232.8737255 |
| HS_from_MQbrain_1877_r | 232.7961089 |
| HS_from_MQbrain_644_l | 232.7594872 |
| HS_10_l | 232.7452901 |
| HS_from_MQbrain_1848_l | 232.6567474 |
| HSbrain_613_l | 232.5256391 |
| HS_from_MQbrain_363_l | 232.4863871 |
| Hsd_from_Mmd_353_l | 232.4164195 |
| HS_from_MQbrain_1260_l | 232.4018345 |
| HS_from_MQbrain_69_r | 232.3221693 |
| HS_from_MQbrain_1190_r | 232.298263 |
| HS_from_MQbrain_1759_r | 232.1142508 |
| HS_275_l | 232.0800842 |
| HSbrain_74_l | 231.9729611 |
| HS_from_MQbrain_1873_r | 231.9697734 |
| HSbrain_307_r | 231.9591602 |
| HSbrain_749_r | 231.9379385 |
| HS_from_MQbrain_386_r | 231.6815526 |
| HS_from_MQbrain_1387_l | 231.6723487 |
| HS_from_MQbrain_1661_l | 231.520072 |
| HSbrain_440_l | 231.4604735 |
| HSbrain_690_l | 231.2618802 |
| HS_from_MQbrain_370_r | 231.2423353 |
| HS_121_r | 231.1610876 |
| HS_from_MQbrain_1447_r | 231.1590957 |
| Hsd_82_l | 231.1408694 |
| HS_from_MQbrain_591_l | 230.9251337 |
| HS_from_MQbrain_1437_r | 230.8888026 |
| Hsd_72_r | 230.8138644 |
| HS_from_MQbrain_951_l | 230.432556 |
| HS_from_PT_295_l | 230.1962907 |
| Hsd_from_Mmd_123_l | 230.1089517 |
| HS_from_MQbrain_722_r | 230.0329269 |
| HSbrain_612_l | 229.9874033 |
| HS_from_MQbrain_20_r | 229.8506554 |
| Hsd_from_Mmd_95_l | 229.7701711 |
| HS_from_MQbrain_973_l | 229.669574 |
| HSbrain_70_l | 229.5640633 |
| Hsd_111_l | 229.5515148 |
| HS_from_MM_305_l | 229.466534 |
| HS_240_r | 229.4510162 |
| HS_from_MQbrain_840_l | 229.3759793 |
| HS_from_MQbrain_1668_l | 229.3696849 |
| HS_from_MQbrain_372_l | 229.299134 |
| HS_from_MQbrain_1542_r | 229.2584359 |
| HSbrain_708_l | 229.246264 |
| HSbrain_808_l | 229.1617442 |
| HS_from_MQbrain_1891_l | 228.912446 |
| HSbrain_527_r | 228.8704808 |
| HS_305_l | 228.659446 |
| HSbrain_769_r | 228.6374424 |
| HS_from_MQbrain_911_r | 228.6253218 |
| HS_from_MQbrain_553_l | 228.565067 |
| HS_from_PT_325_r | 228.4703996 |
| HSbrain_793_r | 228.4549548 |
| HSbrain_457_l | 228.4521117 |
| HS_217_l | 228.449758 |
| HSbrain_420_r | 228.3614012 |
| HS_from_MQbrain_788_r | 228.3312808 |
| HS_from_MQbrain_845_r | 228.2389891 |
| Hsd_from_Mmd_371_l | 228.1350664 |
| HS_from_MQbrain_1400_l | 228.1061615 |
| HSbrain_182_r | 228.0472357 |
| HS_from_MM_179_l | 228.0284987 |
| HS_from_MQbrain_1696_r | 227.6895821 |
| HS_from_MQbrain_1631_r | 227.5696524 |
| HSbrain_8_r | 227.4862458 |
| HS_from_PT_112_l | 227.4432793 |
| HS_from_MQbrain_618_r | 227.3219021 |
| HS_from_MQbrain_1120_l | 227.2890626 |
| HS_from_MQbrain_1741_l | 227.287559 |
| HS_from_MQbrain_1360_l | 227.2860027 |
| HSbrain_514_l | 227.2074795 |
| HS_from_MQbrain_1794_r | 227.1557202 |
| HS_from_MQbrain_1869_l | 227.1108561 |
| HSbrain_767_r | 227.1006295 |
| Hsd_from_Mmd_157_l | 227.0715861 |
| HSbrain_75_l | 227.0689221 |
| HSbrain_220_l | 226.9273128 |
| HSbrain_155_l | 226.8778631 |
| HS_from_MQbrain_442_r | 226.8297266 |
| HSbrain_150_r | 226.7664934 |
| HS_from_MQbrain_900_l | 226.6983928 |
| HS_from_MM_273_l | 226.6169054 |
| HS_227_r | 226.5480615 |
| HS_173_r | 226.5087437 |
| HS_from_MQbrain_1719_r | 226.4594554 |
| HSbrain_306_r | 226.3244274 |
| HS_from_MQbrain_1471_l | 226.2752381 |
| HS_from_MQbrain_1323_r | 226.2120415 |
| HSbrain_227_r | 226.199823 |
| HSbrain_359_r | 226.1695269 |
| Hsd_from_Mmd_147_l | 226.1156692 |
| HSbrain_301_l | 226.109583 |
| HSbrain_231_l | 226.0585846 |
| HS_from_MQbrain_221_l | 226.0465685 |
| HSbrain_45_r | 226.0306347 |
| HS_from_MQbrain_1768_l | 226.0249343 |
| HS_135_r | 225.9650344 |
| HS_from_MQbrain_74_r | 225.8922409 |
| HSbrain_713_l | 225.8656496 |
| HS_from_MQbrain_369_r | 225.8273521 |
| HSbrain_67_l | 225.7697699 |
| HS_from_MQbrain_1239_l | 225.7278011 |
| HSbrain_510_l | 225.5054446 |
| HS_from_MQbrain_428_r | 225.496121 |
| HSbrain_188_r | 225.4205714 |
| HSbrain_226_r | 225.405043 |
| HS_from_MQbrain_533_l | 225.4036486 |
| HS_from_MQbrain_1215_r | 225.3953662 |
| HS_from_MM_174_r | 225.3789785 |
| HS_from_MQbrain_47_l | 225.3526192 |
| HS_from_MQbrain_472_r | 225.3108408 |
| HSbrain_513_r | 225.3093848 |
| HS_from_MQbrain_810_l | 225.1889153 |
| HS_300_l | 225.109114 |
| HS_from_MQbrain_249_l | 225.0944779 |
| HSbrain_90_l | 225.0706303 |
| HS_from_MQbrain_707_l | 224.9895326 |
| HS_from_MQbrain_1641_l | 224.9752825 |
| HSbrain_1210_r | 224.6749935 |
| HS_34_r | 224.6642585 |
| HS_from_MQbrain_612_r | 224.6357358 |
| HSbrain_144_r | 224.6332678 |
| HS_from_MQbrain_1599_l | 224.6258779 |
| HS_from_MQbrain_1553_r | 224.6151091 |
| HSbrain_54_l | 224.5049166 |
| HS_from_MQbrain_903_r | 224.4989842 |
| HS_from_MQbrain_203_l | 224.3395248 |
| HSbrain_391_l | 224.3011525 |
| HS_from_MQbrain_1084_r | 224.2471514 |
| HSbrain_212_l | 224.1025327 |
| HSbrain_148_r | 224.0526161 |
| HSbrain_183_r | 224.0444529 |
| HS_from_MQbrain_1001_r | 223.8116976 |
| HS_from_MM_171_r | 223.7805313 |
| HS_from_MQbrain_1393_r | 223.7656716 |
| HSbrain_677_l | 223.7296488 |
| HS_from_MQbrain_1608_l | 223.6803882 |
| HS_from_MQbrain_1279_r | 223.6755379 |
| HS_from_MQbrain_1318_l | 223.6306158 |
| HS_142_l | 223.5805612 |
| HS_from_MQbrain_1398_l | 223.4944603 |
| HS_from_MQbrain_278_l | 223.3803128 |
| HS_from_PT_258_l | 223.3578239 |
| HSbrain_665_l | 223.3549301 |
| HSbrain_194_l | 223.3237532 |
| HS_from_MQbrain_828_l | 223.2821181 |
| HS_from_MQbrain_1353_r | 223.0961628 |
| HSbrain_537_r | 223.0610428 |
| HS_from_MM_176_r | 222.9246348 |
| HS_from_MQbrain_40_r | 222.7083501 |
| HS_from_MQbrain_54_r | 222.6947248 |
| HS_from_MQbrain_219_l | 222.5900237 |
| Hsd_29_r | 222.5746007 |
| HSbrain_664_r | 222.5408696 |
| HS_from_MQbrain_28_r | 222.5267448 |
| HSbrain_879_r | 222.3891398 |
| HSbrain_110_r | 222.3856849 |
| HS_from_MQbrain_459_l | 222.3828955 |
| HSbrain_186_r | 222.3706738 |
| HS_from_MM_335_r | 222.293289 |
| HSbrain_593_r | 222.0517315 |
| HSbrain_18_l | 222.0054178 |
| HSbrain_785_r | 221.9470784 |
| HSbrain_318_l | 221.848446 |
| HS_from_MQbrain_56_l | 221.5946492 |
| HS_from_PT_27_r | 221.573516 |
| HS_from_PT_121_r | 221.5383439 |
| HSbrain_560_l | 221.4874069 |
| HS_from_MQbrain_1345_l | 221.4839913 |
| Hsd_from_Mmd_430_r | 221.4427335 |
| HS_from_MQbrain_1241_l | 221.397103 |
| HS_from_MM_80_l | 221.3749747 |
| HS_from_MQbrain_1621_r | 221.3394861 |
| HS_166_l | 221.2754845 |
| HS_83_l | 221.1440492 |
| HS_from_MM_338_l | 221.1267321 |
| Hsd_71_l | 221.0863989 |
| HS_from_PT_64_r | 221.0400632 |
| HS_from_MQbrain_448_l | 221.0380546 |
| HS_from_MQbrain_1807_l | 220.9858095 |
| HS_170_r | 220.9849091 |
| HSbrain_735_l | 220.9821714 |
| Hsd_from_Mmd_100_l | 220.9485794 |
| HSbrain_209_r | 220.8283824 |
| HS_from_MQbrain_1645_r | 220.7719103 |
| HS_from_MQbrain_919_r | 220.7669007 |
| HS_from_MQbrain_1027_r | 220.7446535 |
| HS_from_MQbrain_1259_l | 220.6733716 |
| HS_from_MQbrain_1095_r | 220.5708398 |
| HSbrain_610_r | 220.5552561 |
| HS_from_PT_163_r | 220.5509774 |
| HS_from_MQbrain_806_l | 220.5421957 |
| Hsd_83_r | 220.5378697 |
| HS_from_MQbrain_1545_l | 220.4322592 |
| HSbrain_1409_r | 220.3723662 |
| HS_from_MQbrain_171_r | 220.3354018 |
| HS_from_MQbrain_99_r | 220.3197222 |
| HS_from_MQbrain_97_r | 220.2493082 |
| HSbrain_483_l | 220.2067855 |
| HS_from_PT_56_r | 220.1817978 |
| HSbrain_738_r | 220.0942656 |
| HSbrain_335_l | 220.0028261 |
| HS_44_l | 219.9850868 |
| HSbrain_434_l | 219.982723 |
| HSbrain_419_l | 219.8793357 |
| HS_from_MQbrain_706_l | 219.6839321 |
| HSbrain_305_r | 219.5650075 |
| HS_from_MQbrain_297_l | 219.3559964 |
| HS_from_MQbrain_1814_r | 219.2553574 |
| HS_from_MQbrain_391_r | 219.2029092 |
| HSbrain_479_l | 219.1097463 |
| HS_from_MQbrain_1160_l | 219.0375254 |
| HS_283_r | 219.0305275 |
| HS_from_MQbrain_1454_l | 218.93675 |
| HS_256_r | 218.9200582 |
| HS_from_MQbrain_1810_l | 218.9019542 |
| HS_from_MQbrain_724_l | 218.8401444 |
| HSbrain_676_l | 218.8004656 |
| HSbrain_386_l | 218.7869488 |
| HS_from_MQbrain_427_l | 218.7011984 |
| Hsd_from_Mmd_96_l | 218.6585211 |
| HS_from_MQbrain_650_r | 218.5666187 |
| HSbrain_740_r | 218.5007167 |
| HS_from_MQbrain_1116_l | 218.2961635 |
| HS_from_MQbrain_272_l | 218.2733075 |
| HSbrain_210_l | 218.1425564 |
| Hsd_from_Mmd_526_r | 218.1302929 |
| HS_from_MQbrain_1364_l | 218.0461843 |
| HS_from_MQbrain_1379_l | 217.8911784 |
| HSbrain_547_r | 217.8399451 |
| HS_from_MQbrain_1750_r | 217.7023298 |
| HS_from_MQbrain_421_r | 217.599171 |
| HS_165_r | 217.5678412 |
| Hsd_from_Mmd_245_r | 217.480266 |
| HS_from_MQbrain_664_l | 217.4766553 |
| HS_from_MQbrain_538_l | 217.3956368 |
| HS_from_MQbrain_626_r | 217.3494318 |
| HS_from_MQbrain_1619_r | 217.0923928 |
| HSbrain_204_r | 217.0811451 |
| HS_from_MQbrain_715_l | 216.9853807 |
| HS_from_MQbrain_1090_l | 216.9617848 |
| HSbrain_132_r | 216.9340307 |
| HS_from_MQbrain_935_r | 216.9266428 |
| HSbrain_620_l | 216.6725849 |
| HS_from_MM_14_r | 216.6538843 |
| HSbrain_741_l | 216.6387437 |
| HS_from_MQbrain_648_r | 216.5701764 |
| HSbrain_51_r | 216.5598747 |
| HS_137_r | 216.3781618 |
| Hsd_from_Mmd_20_l | 216.2744272 |
| HSbrain_32_l | 216.2330231 |
| HS_from_MQbrain_1382_l | 216.2287654 |
| HS_from_MQbrain_87_l | 216.1747613 |
| HSbrain_130_l | 216.1273018 |
| HS_161_r | 216.0695424 |
| HS_from_MQbrain_493_r | 215.9987774 |
| HS_from_MQbrain_466_r | 215.8030614 |
| HS_244_l | 215.8010509 |
| HS_from_MQbrain_499_r | 215.7448563 |
| HS_from_MQbrain_1593_l | 215.6965191 |
| HSbrain_1362_l | 215.6819962 |
| HS_from_MQbrain_1665_l | 215.5641963 |
| HS_from_MQbrain_1871_r | 215.4340318 |
| HS_from_PT_207_l | 215.4230025 |
| HS_from_MQbrain_1854_r | 215.3909189 |
| HS_from_MQbrain_1685_l | 215.324619 |
| HSbrain_515_r | 215.15954 |
| HS_from_MQbrain_645_l | 215.060159 |
| HS_245_r | 215.02563 |
| HS_3_l | 215.0085435 |
| HS_from_MQbrain_1388_l | 214.966548 |
| HSbrain_804_r | 214.9357091 |
| HS_from_MQbrain_343_l | 214.8585663 |
| HS_from_MQbrain_1841_r | 214.8445757 |
| Hsd_8_l | 214.8202064 |
| HSbrain_586_r | 214.7720002 |
| HS_from_MQbrain_714_l | 214.7487813 |
| HS_from_MQbrain_1455_l | 214.6991315 |
| HS_from_MQbrain_319_r | 214.6690912 |
| HS_from_PT_294_l | 214.6290251 |
| HS_33_r | 214.5936716 |
| HS_from_MQbrain_1766_r | 214.5034772 |
| HSbrain_627_r | 214.4670798 |
| HS_from_PT_339_l | 214.3823639 |
| HSbrain_33_l | 214.3626675 |
| HS_258_l | 214.3589815 |
| HS_from_MQbrain_901_l | 214.2516994 |
| HS_128_r | 214.1983377 |
| HS_118_l | 214.1827525 |
| HS_from_MQbrain_513_l | 214.0755624 |
| HS_from_MQbrain_1222_r | 214.0598655 |
| HSbrain_267_l | 214.022333 |
| HSbrain_1416_l | 214.0180979 |
| HS_114_l | 214.0153294 |
| HS_from_MM_65_l | 213.9686757 |
| Hsd_90_r | 213.9554125 |
| Hsd_from_Mmd_212_l | 213.9400439 |
| HS_from_MQbrain_1198_l | 213.821992 |
| HS_from_MQbrain_260_l | 213.7960437 |
| HSbrain_288_l | 213.7938445 |
| HS_from_MQbrain_449_l | 213.7795306 |
| HS_from_MQbrain_743_l | 213.7726216 |
| HS_from_MQbrain_890_l | 213.7000098 |
| HS_from_MQbrain_258_l | 213.6672002 |
| Hsd_37_l | 213.5626033 |
| HSbrain_388_r | 213.3920423 |
| HS_from_MQbrain_1363_r | 213.3710646 |
| HSbrain_181_r | 213.2667334 |
| HS_from_MQbrain_609_l | 213.1909341 |
| HSbrain_19_r | 213.1745883 |
| HSbrain_1269_r | 213.1586429 |
| HS_182_l | 213.108874 |
| HS_from_MQbrain_654_r | 212.9463137 |
| HS_from_MQbrain_1864_r | 212.8955561 |
| HSbrain_724_r | 212.8164571 |
| HSbrain_643_l | 212.7527559 |
| HS_from_MQbrain_1663_l | 212.7351272 |
| HS_from_MQbrain_872_l | 212.7235747 |
| HSbrain_639_r | 212.6705004 |
| HSbrain_472_r | 212.582847 |
| HSbrain_812_r | 212.5360978 |
| HS_from_MQbrain_532_r | 212.5327446 |
| HS_from_MQbrain_968_r | 212.4682002 |
| HSbrain_30_l | 212.4321641 |
| HS_from_MQbrain_1366_l | 212.34134 |
| Hsd_from_Mmd_256_l | 212.2108757 |
| HS_from_MQbrain_561_l | 212.1470198 |
| Hsd_from_Mmd_92_r | 212.1087908 |
| HS_113_r | 212.103211 |
| HSbrain_525_l | 212.0061091 |
| HSbrain_176_l | 211.9808586 |
| HS_242_r | 211.9082409 |
| HS_from_MM_346_l | 211.7235677 |
| HS_243_l | 211.6547669 |
| HS_306_l | 211.5536628 |
| HS_from_MQbrain_710_l | 211.5445803 |
| HSbrain_61_r | 211.5356004 |
| HS_from_MQbrain_708_r | 211.4887754 |
| HSbrain_321_r | 211.435012 |
| HSbrain_916_l | 211.4233793 |
| HS_from_PT_229_l | 211.3671417 |
| HS_208_r | 211.3635201 |
| HSbrain_364_r | 211.3556115 |
| HS_155_l | 211.2855729 |
| HS_from_MQbrain_528_l | 211.2420518 |
| HS_226_r | 211.1840201 |
| HS_from_MQbrain_1389_l | 211.1430096 |
| HS_from_MQbrain_452_l | 211.1219111 |
| HSbrain_347_l | 211.107069 |
| HSbrain_217_l | 211.0589069 |
| HSbrain_57_r | 211.0174653 |
| HSbrain_421_r | 210.992031 |
| HSbrain_56_l | 210.9521542 |
| Hsd_31_l | 210.9485674 |
| HS_from_MQbrain_17_r | 210.8657734 |
| HSbrain_385_l | 210.8123385 |
| HS_from_MQbrain_504_l | 210.6839838 |
| HS_from_MQbrain_120_r | 210.6657499 |
| HSbrain_487_r | 210.5369083 |
| HSbrain_656_r | 210.503868 |
| HS_from_MQbrain_712_l | 210.3715709 |
| HS_from_MQbrain_699_r | 210.3435386 |
| HSbrain_743_l | 210.3243357 |
| HS_from_MQbrain_180_r | 210.3209412 |
| HS_from_PT_18_l | 210.1470861 |
| HS_from_MQbrain_85_r | 210.1449333 |
| HS_from_MQbrain_1712_l | 210.0239788 |
| HS_from_MQbrain_1204_r | 209.9240502 |
| HS_from_MQbrain_145_l | 209.7777556 |
| Hsd_61_l | 209.748764 |
| HS_131_r | 209.6414321 |
| HS_from_MQbrain_1273_l | 209.6052632 |
| HSbrain_260_l | 209.5737731 |
| HS_from_MQbrain_1689_l | 209.4623643 |
| HS_from_PT_41_l | 209.4264654 |
| HSbrain_640_r | 209.3752287 |
| HS_from_MQbrain_84_l | 209.3540926 |
| HS_from_MQbrain_1644_l | 209.32 |
| HS_from_MQbrain_467_l | 209.268142 |
| HS_77_l | 209.1463931 |
| HS_from_MQbrain_527_l | 209.0387081 |
| HSbrain_573_l | 209.0095416 |
| Hsd_from_Mmd_450_r | 208.9211404 |
| HSbrain_68_r | 208.8873182 |
| HSbrain_96_l | 208.7429867 |
| Hsd_78_l | 208.7314312 |
| HSbrain_597_r | 208.6310714 |
| HS_from_MQbrain_1184_r | 208.6202764 |
| HS_from_MQbrain_1885_r | 208.5987571 |
| HS_from_MQbrain_1544_r | 208.3981899 |
| HS_from_MQbrain_86_l | 208.3164223 |
| Hsd_62_l | 208.0883252 |
| HS_from_MM_182_r | 208.0844095 |
| HS_from_MM_309_r | 208.0343975 |
| HS_from_MQbrain_638_r | 208.030846 |
| HS_from_MQbrain_450_l | 208.0243134 |
| HS_from_PT_146_l | 207.9363128 |
| HS_from_MQbrain_1561_l | 207.9055923 |
| HS_from_MQbrain_783_l | 207.9026032 |
| HS_from_MQbrain_1046_r | 207.8703374 |
| HS_from_MQbrain_1402_l | 207.7391938 |
| HS_from_MQbrain_1557_r | 207.6872074 |
| HSbrain_475_r | 207.6303954 |
| Hsd_from_Mmd_471_l | 207.5771736 |
| HS_from_MQbrain_1493_r | 207.5291507 |
| Hsd_from_Mmd_155_r | 207.3532885 |
| HS_from_MQbrain_1552_l | 207.3050287 |
| HS_from_MQbrain_1718_r | 207.2227537 |
| HS_176_r | 207.1975229 |
| HSbrain_376_r | 207.1809373 |
| HSbrain_772_l | 207.1044984 |
| HS_from_MQbrain_1055_r | 207.0197537 |
| HS_from_MQbrain_160_l | 207.0189821 |
| Hsd_from_Mmd_428_r | 206.995807 |
| HSbrain_136_l | 206.8622185 |
| HSbrain_746_r | 206.8187803 |
| HS_70_l | 206.7755591 |
| HSbrain_589_r | 206.7062924 |
| HS_from_MQbrain_268_l | 206.6561016 |
| HS_from_MQbrain_873_r | 206.5787776 |
| HS_from_MQbrain_1795_l | 206.4850267 |
| HS_from_MQbrain_1348_r | 206.3390148 |
| Hsd_from_Mmd_180_l | 206.276727 |
| HSbrain_400_r | 206.2134583 |
| HSbrain_146_r | 206.1538175 |
| HSbrain_72_l | 206.0852876 |
| HS_159_r | 206.0784814 |
| HS_from_MQbrain_1507_l | 205.841515 |
| HS_102_l | 205.6195359 |
| HS_from_MQbrain_691_r | 205.3284172 |
| HSbrain_396_l | 205.3057539 |
| HS_from_PT_283_l | 205.2617999 |
| HS_from_MQbrain_1776_l | 205.2456623 |
| Hsd_from_Mmd_530_r | 205.2141219 |
| HS_from_MQbrain_1221_r | 205.1595375 |
| HS_from_MQbrain_313_r | 205.1377292 |
| HS_from_MQbrain_234_r | 205.0056018 |
| HSbrain_304_l | 204.8368425 |
| HS_from_MQbrain_150_l | 204.8007622 |
| HS_from_MQbrain_1266_r | 204.7418882 |
| HS_from_MQbrain_569_r | 204.738491 |
| HS_from_MQbrain_1433_r | 204.5305106 |
| HS_from_MQbrain_414_r | 204.4830788 |
| HS_from_MQbrain_802_l | 204.4242655 |
| HS_from_MQbrain_147_r | 204.3734082 |
| HS_from_MQbrain_1413_l | 204.3272671 |
| HS_from_MQbrain_1695_r | 204.2688796 |
| HS_from_MQbrain_210_r | 204.1332639 |
| HS_from_MM_321_r | 203.9926622 |
| HS_186_r | 203.9246728 |
| HS_from_MM_306_r | 203.8838871 |
| HS_from_MM_44_l | 203.8131364 |
| HSbrain_50_l | 203.7449536 |
| HS_79_r | 203.7167335 |
| HS_from_MM_192_r | 203.6417757 |
| HS_from_MQbrain_1074_l | 203.636716 |
| HS_from_MQbrain_621_r | 203.6320108 |
| HSbrain_461_l | 203.4862371 |
| HSbrain_123_l | 203.4486322 |
| HSbrain_65_r | 203.2999735 |
| HS_287_l | 203.2812692 |
| HSbrain_516_r | 203.206938 |
| HS_from_MQbrain_1036_r | 203.1951911 |
| HS_from_MQbrain_191_l | 203.1756555 |
| HS_from_MQbrain_1193_r | 203.1490153 |
| HS_from_MQbrain_2633_l | 203.0852846 |
| HSbrain_64_r | 203.067999 |
| HSbrain_578_r | 203.0612339 |
| HSbrain_138_l | 202.9635847 |
| HS_from_MQbrain_9_r | 202.9562969 |
| HS_from_PT_248_l | 202.9458098 |
| HS_from_MM_319_l | 202.6221234 |
| HS_from_PT_70_r | 202.4337957 |
| HSbrain_55_r | 202.4300733 |
| Hsd_64_r | 202.1734743 |
| HS_46_r | 202.1680761 |
| HS_from_MQbrain_131_r | 202.0595323 |
| HS_from_MQbrain_2689_l | 202.035201 |
| HS_from_PT_211_l | 201.8006639 |
| HS_from_MQbrain_1676_r | 201.7301962 |
| HS_from_MQbrain_1767_l | 201.6583037 |
| HSbrain_699_r | 201.6052561 |
| HS_29_l | 201.5999875 |
| HS_from_MQbrain_882_r | 201.5350781 |
| HS_150_l | 201.5054656 |
| HSbrain_691_l | 201.4670994 |
| HS_from_MQbrain_1303_r | 201.3209506 |
| HS_from_PT_220_l | 201.2064584 |
| HS_136_l | 201.0664033 |
| Hsd_from_Mmd_456_l | 200.9559735 |
| HS_from_MQbrain_545_l | 200.7706369 |
| Hsd_77_r | 200.6733718 |
| HSbrain_23_r | 200.6525784 |
| HSbrain_63_l | 200.6399322 |
| HSbrain_84_l | 200.5841432 |
| HSbrain_1431_l | 200.5748738 |
| HS_180_r | 200.4582411 |
| HSbrain_91_l | 200.4180855 |
| HSbrain_102_r | 200.4155278 |
| HSbrain_153_l | 200.2905692 |
| HSbrain_526_r | 200.2642793 |
| HS_109_l | 200.1919676 |
| HSbrain_126_l | 200.1325332 |
| HS_from_MQbrain_1283_l | 200.1271847 |
| HS_from_MQbrain_405_l | 200.083515 |
| Hsd_from_Mmd_330_l | 200.078931 |
| Hsd_from_Mmd_429_l | 200.0012831 |
| Hsd_from_Mmd_438_r | 199.9189326 |
| HSbrain_463_l | 199.836684 |
| HSbrain_193_l | 199.7083811 |
| HSbrain_289_l | 199.6939274 |
| HS_from_MQbrain_1717_l | 199.667607 |
| HS_from_MM_79_l | 199.5386163 |
| HS_from_PT_38_r | 199.4482749 |
| Hsd_from_Mmd_470_l | 199.418794 |
| HSbrain_373_l | 199.3527183 |
| HS_35_l | 199.2603922 |
| HSbrain_720_r | 199.2261851 |
| HSbrain_124_l | 199.2157195 |
| HSbrain_275_l | 199.2030892 |
| HS_from_MQbrain_943_l | 199.1939949 |
| HS_from_MQbrain_1456_l | 199.1014452 |
| HSbrain_642_r | 198.8901506 |
| HS_from_MM_112_r | 198.8560904 |
| HSbrain_116_l | 198.8454747 |
| HS_78_l | 198.7048825 |
| HS_from_MQbrain_1183_l | 198.4935006 |
| HS_303_r | 198.3144581 |
| HS_138_l | 198.1359437 |
| HS_from_MQbrain_29_l | 198.1116988 |
| HS_from_MQbrain_311_l | 198.0432726 |
| HS_from_MQbrain_869_l | 197.9550289 |
| HSbrain_341_r | 197.8276264 |
| HS_from_MQbrain_1212_r | 197.7890273 |
| HS_from_MQbrain_1721_l | 197.7591475 |
| HS_from_MQbrain_288_l | 197.7437468 |
| HS_from_MQbrain_693_r | 197.7309441 |
| HSbrain_31_l | 197.7117785 |
| HSbrain_459_l | 197.6806078 |
| HS_from_MM_292_r | 197.6376855 |
| HS_from_MQbrain_281_l | 197.384259 |
| HS_from_MM_350_l | 197.3458346 |
| HS_106_l | 197.2533991 |
| Hsd_from_Mmd_176_l | 197.2483957 |
| Hsd_58_l | 197.2050795 |
| HS_from_MQbrain_14_r | 197.1450347 |
| HS_from_MQbrain_1863_l | 197.0519986 |
| HS_from_MM_126_l | 197.0061583 |
| HS_from_MQbrain_508_l | 196.9051127 |
| HS_from_MM_5_r | 196.7804505 |
| HS_from_MQbrain_1288_l | 196.6983091 |
| HS_94_r | 196.6181992 |
| HSbrain_1263_r | 196.5803079 |
| HS_from_MQbrain_497_l | 196.4669787 |
| HS_259_r | 196.3066632 |
| HS_from_MQbrain_1395_r | 196.1672376 |
| HS_146_l | 196.0465543 |
| HS_from_MQbrain_1785_r | 196.0389525 |
| HS_from_MM_304_r | 195.9761008 |
| HS_from_MQbrain_1802_l | 195.9391667 |
| HSbrain_801_r | 195.7930985 |
| HS_from_MQbrain_936_r | 195.7842152 |
| HSbrain_175_l | 195.7623447 |
| Hsd_105_r | 195.7175747 |
| HS_from_MM_150_l | 195.706453 |
| HSbrain_616_r | 195.4635888 |
| HS_from_MQbrain_613_l | 195.43094 |
| HS_183_l | 195.3964293 |
| HS_from_MQbrain_1480_r | 195.3327536 |
| HSbrain_517_l | 195.2983762 |
| HSbrain_1407_l | 195.1893199 |
| HSbrain_350_l | 195.1163033 |
| HS_from_MQbrain_629_l | 195.1102679 |
| HSbrain_562_r | 195.1029577 |
| HSbrain_114_l | 194.9449919 |
| HSbrain_1236_l | 194.855173 |
| HS_from_MQbrain_228_r | 194.8222137 |
| HS_from_MQbrain_128_r | 194.6783323 |
| HS_74_l | 194.632323 |
| HS_from_MQbrain_922_r | 194.5272878 |
| HS_from_MQbrain_1298_r | 194.5026983 |
| HS_from_MM_266_r | 194.4374232 |
| Hsd_32_l | 194.403086 |
| HS_from_PT_161_r | 194.3462227 |
| HS_from_MQbrain_818_l | 194.3299932 |
| HS_from_MQbrain_1088_l | 194.3140687 |
| HSbrain_630_l | 194.2979437 |
| HS_from_MQbrain_379_l | 194.2799743 |
| Hsd_from_Mmd_31_l | 194.1021626 |
| HSbrain_570_l | 194.0529436 |
| HS_from_MM_165_l | 194.0305909 |
| HS_from_MQbrain_1423_l | 193.964167 |
| HSbrain_248_l | 193.8830045 |
| Hsd_from_Mmd_94_l | 193.7657691 |
| HSbrain_162_l | 193.6282482 |
| HS_from_MQbrain_1700_l | 193.6041577 |
| HSbrain_360_r | 193.590164 |
| HS_from_MQbrain_1573_l | 193.4803504 |
| HS_116_l | 193.4215677 |
| HS_from_PT_218_r | 193.3522736 |
| HS_from_MQbrain_271_r | 193.3227472 |
| HS_from_MQbrain_222_l | 193.1226274 |
| HS_from_MQbrain_1548_l | 193.0711482 |
| HSbrain_362_l | 192.9404754 |
| HSbrain_1174_l | 192.7667477 |
| HS_from_MQbrain_1478_l | 192.6931886 |
| HS_from_MQbrain_1346_l | 192.6458383 |
| HSbrain_599_l | 192.6183943 |
| Hsd_from_Mmd_413_l | 192.5190937 |
| Hsd_from_Mmd_33_r | 192.4364738 |
| HS_from_MQbrain_403_l | 192.404673 |
| HS_from_MQbrain_1624_l | 192.3884805 |
| HS_from_MQbrain_1112_r | 192.3632878 |
| Hsd_from_Mmd_172_l | 192.143029 |
| HS_from_MM_299_r | 192.1411625 |
| HS_from_MQbrain_921_l | 192.1335465 |
| HS_from_MQbrain_1818_r | 192.1179163 |
| HS_from_MQbrain_658_r | 192.0124629 |
| HS_from_MQbrain_674_r | 191.9044513 |
| Hsd_from_Mmd_383_l | 191.7899174 |
| Hsd_63_r | 191.7137571 |
| HS_from_MQbrain_885_r | 191.5324794 |
| HS_from_MQbrain_58_r | 191.522255 |
| HS_from_MQbrain_1745_r | 191.5196529 |
| HS_from_MQbrain_1569_l | 191.4989985 |
| HSbrain_752_l | 191.3899851 |
| HSbrain_635_r | 191.3323113 |
| HSbrain_313_r | 191.3288742 |
| HS_from_MQbrain_535_l | 191.3094563 |
| HS_from_MQbrain_1361_l | 191.1427072 |
| HS_from_PT_204_r | 191.0832874 |
| HSbrain_352_l | 191.0286866 |
| HS_9_l | 191.0245696 |
| HS_from_MQbrain_825_l | 191.0142911 |
| HSbrain_590_r | 190.8385095 |
| HS_from_MQbrain_1311_l | 190.8001726 |
| HSbrain_651_r | 190.7821606 |
| HSbrain_488_l | 190.6199449 |
| HSbrain_324_l | 190.5828506 |
| HS_201_l | 190.5393273 |
| HS_from_MQbrain_464_l | 190.5025514 |
| HS_90_l | 190.4770169 |
| HS_185_l | 190.419054 |
| HS_from_MQbrain_529_r | 190.4031906 |
| HS_12_l | 190.383325 |
| HSbrain_223_r | 190.3224269 |
| HS_from_MM_6_l | 190.1275732 |
| Hsd_from_Mmd_519_r | 190.1006667 |
| HS_from_MQbrain_894_l | 190.0849242 |
| HSbrain_164_r | 189.9523775 |
| Hsd_from_Mmd_433_r | 189.8742828 |
| HSbrain_528_r | 189.872647 |
| HS_from_MQbrain_666_r | 189.8608917 |
| HSbrain_375_r | 189.8348043 |
| HS_from_MQbrain_1145_r | 189.8259778 |
| HS_from_MQbrain_881_l | 189.779606 |
| HS_from_MQbrain_955_l | 189.6235242 |
| HS_from_MQbrain_350_l | 189.461419 |
| HS_from_MQbrain_1888_r | 189.3084792 |
| HS_from_MQbrain_406_r | 189.233991 |
| HS_126_r | 189.1558613 |
| Hsd_from_Mmd_416_l | 189.0597441 |
| HS_from_MQbrain_1243_r | 189.054122 |
| HS_from_MQbrain_981_l | 188.9895746 |
| HSbrain_659_r | 188.9836496 |
| HS_from_MQbrain_1280_l | 188.9766392 |
| HS_from_MQbrain_932_r | 188.8437058 |
| HSbrain_172_r | 188.8196379 |
| HS_from_MQbrain_1730_r | 188.7832532 |
| HS_from_MQbrain_291_l | 188.7078132 |
| HS_from_MQbrain_485_r | 188.6613768 |
| HSbrain_280_l | 188.6563025 |
| HS_from_MQbrain_1674_l | 188.625056 |
| HS_from_MQbrain_870_l | 188.497046 |
| HS_from_PT_20_r | 188.3393313 |
| HSbrain_592_r | 188.3237767 |
| HS_from_MQbrain_383_l | 188.3158756 |
| HS_from_MQbrain_1175_r | 188.3144325 |
| HS_from_PT_162_r | 188.2819118 |
| HSbrain_786_r | 188.2594341 |
| HSbrain_348_l | 188.1407447 |
| Hsd_99_r | 188.1035755 |
| HS_17_r | 187.9339119 |
| HS_from_PT_95_r | 187.7990727 |
| HS_from_MQbrain_1659_r | 187.7357442 |
| HS_from_MQbrain_39_r | 187.6304469 |
| HS_from_MQbrain_1844_r | 187.6108805 |
| HSbrain_710_r | 187.4520996 |
| HS_from_MQbrain_2380_r | 187.2947993 |
| HS_from_MQbrain_1834_r | 187.1781356 |
| HS_from_MQbrain_22_r | 187.1051151 |
| HSbrain_594_l | 186.9175668 |
| HS_from_MQbrain_83_l | 186.9062136 |
| HS_from_MQbrain_433_l | 186.8433165 |
| HS_from_MQbrain_1062_r | 186.7629184 |
| HSbrain_495_l | 186.7445849 |
| HS_from_MQbrain_918_l | 186.6733108 |
| HS_from_MQbrain_1852_r | 186.6102831 |
| HS_from_MQbrain_57_l | 186.5917929 |
| HSbrain_1066_l | 186.5290013 |
| HS_4_r | 186.5048701 |
| HS_from_MM_49_l | 186.4616319 |
| Hsd_from_Mmd_93_l | 186.4505924 |
| HSbrain_474_l | 186.4221505 |
| HS_215_l | 186.3533099 |
| HS_from_MQbrain_1839_l | 186.2075177 |
| HSbrain_564_r | 186.0420534 |
| HS_54_l | 185.9367326 |
| HS_from_MM_251_l | 185.8137117 |
| HS_from_MQbrain_1566_l | 185.7444631 |
| HS_from_MQbrain_25_r | 185.7440636 |
| HS_from_MQbrain_186_r | 185.6953187 |
| HSbrain_805_l | 185.674964 |
| HS_from_MQbrain_1383_r | 185.5897107 |
| HS_from_MQbrain_332_r | 185.5839917 |
| HSbrain_771_l | 185.5150257 |
| HSbrain_591_l | 185.5052057 |
| HSbrain_548_l | 185.4850722 |
| HS_51_l | 185.3456287 |
| HS_from_PT_170_l | 185.2808375 |
| HS_from_MM_70_r | 185.0757794 |
| HS_from_MQbrain_1422_r | 184.9614981 |
| HS_76_l | 184.9599379 |
| HSbrain_675_l | 184.9297939 |
| HS_290_l | 184.9148333 |
| HS_from_MQbrain_347_l | 184.9027155 |
| HS_from_MQbrain_631_l | 184.728 |
| HSbrain_1168_l | 184.7243845 |
| Hsd_from_Mmd_261_l | 184.5887939 |
| Hsd_65_r | 184.3852713 |
| HS_from_MQbrain_1061_l | 184.3228969 |
| HS_from_MM_161_l | 184.2770352 |
| HS_from_MM_191_l | 184.213433 |
| HS_from_MQbrain_63_l | 184.1670148 |
| HS_from_MQbrain_401_r | 184.1230274 |
| Hsd_from_Mmd_85_r | 184.0986177 |
| HS_from_MQbrain_640_l | 183.9955145 |
| HS_from_PT_25_l | 183.9374174 |
| HS_from_PT_31_r | 183.634517 |
| HS_107_l | 183.5837434 |
| HS_133_l | 183.5586928 |
| HS_from_MQbrain_740_l | 183.5545321 |
| HS_from_MM_123_r | 183.4779998 |
| HSbrain_38_r | 183.4285032 |
| HSbrain_596_r | 183.4234846 |
| HSbrain_383_r | 183.4226045 |
| HS_from_MQbrain_1565_r | 183.33017 |
| HS_152_l | 183.0697159 |
| Hsd_from_Mmd_169_r | 182.7898422 |
| HS_from_MQbrain_273_l | 182.7573142 |
| HS_257_r | 182.6194298 |
| Hsd_43_l | 182.6106383 |
| HSbrain_300_l | 182.522588 |
| HS_from_MQbrain_216_l | 182.4516418 |
| HSbrain_435_r | 182.3583857 |
| HSbrain_502_l | 182.3121978 |
| HS_from_MQbrain_659_l | 182.2736817 |
| Hsd_from_Mmd_80_r | 182.267229 |
| HSbrain_243_l | 182.2549801 |
| HSbrain_185_l | 182.1563176 |
| HS_from_MQbrain_1859_l | 181.9056424 |
| HS_91_r | 181.8851129 |
| HS_from_MQbrain_1315_l | 181.848048 |
| Hsd_108_r | 181.7633595 |
| HS_from_MQbrain_1427_l | 181.7148181 |
| HSbrain_503_r | 181.710235 |
| HS_from_MQbrain_592_l | 181.6866459 |
| HS_from_MQbrain_1602_r | 181.6482095 |
| HSbrain_346_l | 181.6254174 |
| HS_from_MQbrain_819_l | 181.598646 |
| HS_196_l | 181.591622 |
| HS_from_PT_234_l | 181.4676297 |
| HS_from_MM_307_l | 181.4274115 |
| HSbrain_135_r | 181.3044184 |
| HS_from_PT_71_l | 181.2957273 |
| HS_from_MQbrain_1_l | 181.2744529 |
| HSbrain_606_l | 181.1992813 |
| HSbrain_165_l | 181.1832002 |
| HS_from_MQbrain_567_l | 181.141058 |
| Hsd_79_l | 181.0929049 |
| Hsd_from_Mmd_129_r | 181.0451576 |
| HS_from_MQbrain_931_r | 180.928267 |
| HS_from_PT_310_l | 180.8331695 |
| HS_from_MQbrain_1051_l | 180.7987744 |
| HSbrain_255_l | 180.7521066 |
| HS_from_MQbrain_324_r | 180.6614293 |
| HSbrain_716_l | 180.5676912 |
| Hsd_from_Mmd_189_l | 180.4050764 |
| Hsd_from_Mmd_225_r | 180.272844 |
| HS_from_MQbrain_1484_l | 180.1270647 |
| HSbrain_722_l | 180.1260425 |
| HSbrain_133_l | 180.0609243 |
| HSbrain_71_l | 180.0336735 |
| HS_from_MQbrain_883_r | 179.9509263 |
| HS_from_MQbrain_1229_r | 179.8888306 |
| HSbrain_187_r | 179.7554723 |
| HSbrain_1420_l | 179.7305997 |
| HS_from_PT_255_l | 179.6028399 |
| HS_from_MQbrain_1831_r | 179.507715 |
| HS_from_MQbrain_1443_r | 179.1434855 |
| HSbrain_125_r | 179.1019231 |
| HS_125_l | 178.9732196 |
| HS_1_r | 178.872361 |
| HSbrain_762_r | 178.772043 |
| HSbrain_211_l | 178.6326198 |
| HS_from_MM_316_r | 178.5854649 |
| HS_from_MQbrain_90_r | 178.3364821 |
| Hsd_from_Mmd_418_r | 178.3089976 |
| HSbrain_455_r | 178.2419961 |
| HSbrain_917_l | 178.1737031 |
| HS_from_MQbrain_148_l | 178.0297049 |
| HS_from_MQbrain_1235_r | 177.9273987 |
| HS_from_MQbrain_1481_r | 177.8686795 |
| HS_178_r | 177.8554461 |
| HS_15_l | 177.8515513 |
| Hsd_from_Mmd_101_r | 177.7313602 |
| HS_from_MQbrain_265_r | 177.7026523 |
| HS_from_MQbrain_1670_r | 177.6818982 |
| HS_from_MQbrain_1551_l | 177.6424473 |
| HS_from_MQbrain_1199_l | 177.5515382 |
| HSbrain_608_r | 177.4299183 |
| HS_from_MM_91_r | 177.3134135 |
| HSbrain_112_l | 177.2992267 |
| HS_from_MQbrain_969_r | 177.1203053 |
| HS_from_PT_338_r | 177.0554098 |
| HS_from_MQbrain_1070_l | 176.9299998 |
| HS_from_MQbrain_1649_l | 176.9183979 |
| HS_from_MQbrain_1374_l | 176.8739274 |
| Hsd_46_r | 176.8539101 |
| HSbrain_69_r | 176.5948307 |
| HS_from_MM_12_l | 176.3473845 |
| HSbrain_430_r | 176.1827887 |
| HSbrain_470_l | 176.1759915 |
| HS_from_MQbrain_948_r | 176.1262044 |
| HSbrain_751_l | 176.064141 |
| HSbrain_403_r | 175.9332671 |
| HS_from_MQbrain_1678_l | 175.9249049 |
| HSbrain_622_l | 175.8456587 |
| HSbrain_1363_l | 175.794645 |
| HSbrain_601_r | 175.7896102 |
| HSbrain_595_r | 175.7116832 |
| HS_from_MQbrain_388_r | 175.6904319 |
| Hsd_from_Mmd_183_l | 175.6720566 |
| HSbrain_282_l | 175.6138018 |
| HS_from_MQbrain_557_l | 175.6113323 |
| Hsd_102_l | 175.5645249 |
| HSbrain_79_l | 175.383712 |
| HS_299_l | 175.2031924 |
| HS_from_MQbrain_1322_l | 175.1994814 |
| HS_from_MQbrain_322_l | 174.888304 |
| HS_88_l | 174.7354278 |
| HS_from_MM_226_r | 174.3878926 |
| HS_from_MQbrain_1415_l | 174.2147826 |
| HS_143_l | 174.1874239 |
| HSbrain_330_l | 174.1826185 |
| HS_from_MQbrain_878_l | 174.1494734 |
| HS_from_MQbrain_1181_r | 173.9702444 |
| HS_from_MQbrain_239_r | 173.8939829 |
| HS_from_MQbrain_1660_r | 173.8610519 |
| HSbrain_143_l | 173.8367679 |
| HSbrain_768_r | 173.759433 |
| HS_247_l | 173.7058625 |
| HSbrain_696_l | 173.6647619 |
| HS_from_MM_229_r | 173.5953652 |
| HS_from_MQbrain_1372_l | 173.5611463 |
| Hsd_from_Mmd_259_l | 173.2971944 |
| HS_from_MQbrain_1773_l | 173.1308074 |
| HS_from_PT_176_r | 173.0044578 |
| HSbrain_139_l | 172.7598914 |
| HS_from_MQbrain_316_r | 172.2218294 |
| HSbrain_325_l | 172.2192259 |
| HS_from_MQbrain_251_r | 172.0989873 |
| HSbrain_748_r | 171.8187407 |
| HSbrain_258_r | 171.6509812 |
| HS_288_l | 171.5705836 |
| HS_from_MQbrain_37_l | 171.5557838 |
| HSbrain_351_l | 171.4897107 |
| HS_from_MQbrain_385_r | 171.4739179 |
| HS_from_MQbrain_755_r | 171.2982582 |
| Hsd_from_Mmd_91_r | 171.053764 |
| HS_from_MQbrain_1716_r | 170.9229787 |
| HS_from_MQbrain_555_l | 170.9056939 |
| HS_from_MM_199_l | 170.7212373 |
| HSbrain_409_r | 170.7071739 |
| HSbrain_618_r | 170.6474893 |
| HS_from_PT_197_l | 170.5694359 |
| HS_from_MQbrain_1516_l | 170.5258671 |
| HS_from_MQbrain_374_r | 170.5241907 |
| HS_from_MM_242_l | 170.2575956 |
| HS_from_MQbrain_1640_r | 170.1535918 |
| Hsd_from_Mmd_466_l | 169.8854018 |
| HS_from_MQbrain_558_r | 169.8311892 |
| HS_from_PT_250_r | 169.7827673 |
| HS_from_PT_22_l | 169.7548575 |
| HSbrain_712_l | 169.7417362 |
| HS_from_MQbrain_1612_l | 169.5239608 |
| HS_from_MQbrain_1592_l | 169.5192576 |
| HSbrain_158_l | 169.343928 |
| HS_from_MQbrain_1091_l | 169.2750909 |
| HS_from_PT_256_l | 169.2425116 |
| HSbrain_232_l | 169.1546875 |
| HS_from_MQbrain_572_r | 168.9083431 |
| HSbrain_464_l | 168.8249078 |
| HS_from_PT_135_l | 168.7144836 |
| HS_from_MQbrain_669_l | 168.6377915 |
| HS_277_l | 168.4332786 |
| Hsd_from_Mmd_488_l | 168.4228866 |
| HSbrain_450_l | 168.2657456 |
| HSbrain_715_r | 168.0598281 |
| HS_from_MQbrain_1556_l | 168.0497925 |
| HS_from_MQbrain_934_r | 167.9322779 |
| HS_from_MQbrain_930_r | 167.8363796 |
| HS_from_PT_122_l | 167.7150986 |
| HS_from_PT_191_r | 167.6796712 |
| HSbrain_575_l | 167.6062921 |
| HS_from_PT_156_r | 167.4729776 |
| HSbrain_392_l | 166.9784741 |
| HSbrain_892_r | 166.9224826 |
| HS_from_MQbrain_31_l | 166.7933398 |
| HS_from_MQbrain_1450_l | 166.7771396 |
| HS_from_MQbrain_1775_r | 166.5855528 |
| HS_from_MQbrain_1010_l | 166.5364922 |
| Hsd_97_l | 165.882963 |
| Hsd_68_l | 165.8584582 |
| HS_from_MQbrain_26_l | 165.627724 |
| HS_from_MM_334_l | 165.3471372 |
| HS_from_MQbrain_1100_l | 165.2906308 |
| HS_from_MM_38_l | 165.2486961 |
| HSbrain_340_r | 165.2419641 |
| HS_164_r | 165.1061854 |
| Hsd_from_Mmd_492_l | 164.980004 |
| HSbrain_310_l | 164.9263788 |
| HSbrain_807_r | 164.7541547 |
| HS_2_l | 164.7210266 |
| HS_from_MQbrain_774_r | 164.6264718 |
| HSbrain_216_r | 164.5910344 |
| HS_from_MQbrain_1223_l | 164.5470949 |
| Hsd_from_Mmd_193_l | 164.5279208 |
| Hsd_11_l | 164.5093304 |
| HS_from_MQbrain_1150_r | 164.4702221 |
| HS_from_MQbrain_1757_l | 164.4509948 |
| HS_from_MQbrain_1030_l | 164.3970563 |
| HS_from_MQbrain_282_l | 163.9894054 |
| HS_from_MQbrain_1724_l | 163.9516624 |
| HS_from_MQbrain_1590_l | 163.8114142 |
| HS_from_MQbrain_904_l | 163.7759063 |
| HS_from_MQbrain_492_l | 163.6962297 |
| HS_from_MQbrain_1529_r | 163.6110496 |
| HSbrain_524_r | 163.4332942 |
| HSbrain_342_l | 163.2902244 |
| HSbrain_1412_l | 163.2097745 |
| HS_from_MQbrain_1289_l | 163.1398143 |
| HSbrain_121_l | 163.1114217 |
| HS_from_MQbrain_165_l | 162.7040346 |
| HS_from_MQbrain_490_r | 162.3944533 |
| HS_from_MQbrain_1228_r | 162.3489481 |
| HSbrain_448_l | 162.0375436 |
| Hsd_49_r | 162.0054723 |
| Hsd_85_l | 161.9749751 |
| HS_from_MQbrain_346_r | 161.9458653 |
| HSbrain_208_r | 161.7157386 |
| HS_from_MQbrain_45_r | 161.291101 |
| HS_255_l | 161.1405598 |
| HS_from_MQbrain_1985_r | 161.0836782 |
| HS_60_r | 161.0640676 |
| HSbrain_658_r | 160.8896254 |
| Hsd_from_Mmd_468_r | 160.8479695 |
| Hsd_from_Mmd_354_l | 160.6920656 |
| HS_from_MQbrain_1064_r | 160.6505983 |
| Hsd_from_Mmd_228_r | 160.5314694 |
| HS_from_MQbrain_256_l | 160.009102 |
| HS_from_MQbrain_1884_r | 159.9476968 |
| HS_from_MQbrain_1589_r | 159.9022236 |
| HS_from_MQbrain_1002_l | 159.8709917 |
| HSbrain_636_l | 159.8695431 |
| HS_from_MQbrain_834_r | 159.8605199 |
| HS_from_MQbrain_771_r | 159.7398798 |
| HSbrain_369_r | 159.7073641 |
| Hsd_84_l | 159.0858523 |
| HS_from_PT_299_l | 158.8812184 |
| HS_from_MQbrain_1520_r | 158.8609156 |
| HS_from_MQbrain_1128_l | 158.837926 |
| HS_from_MQbrain_1419_r | 158.7663804 |
| HS_from_MQbrain_1740_l | 158.6793211 |
| HS_from_MQbrain_1692_l | 158.6415708 |
| HS_from_MQbrain_21_r | 158.5410847 |
| HS_from_MM_170_l | 158.4309229 |
| HS_from_MQbrain_1684_l | 158.2373516 |
| HS_from_MQbrain_1182_l | 158.0679011 |
| Hsd_93_l | 157.9972237 |
| HSbrain_27_r | 157.9786387 |
| HSbrain_1437_l | 157.9722323 |
| HS_from_MQbrain_207_l | 157.9140691 |
| HS_from_MQbrain_962_l | 157.9046002 |
| HS_from_PT_108_l | 157.8559179 |
| HS_from_MQbrain_1340_l | 157.6981977 |
| Hsd_from_Mmd_174_r | 157.3796566 |
| HSbrain_206_l | 157.3663037 |
| HS_from_MQbrain_1654_l | 157.3073263 |
| HS_from_MQbrain_41_r | 157.2475269 |
| HSbrain_694_r | 157.1044149 |
| Hsd_from_Mmd_499_l | 157.0871197 |
| Hsd_from_Mmd_284_r | 156.9741681 |
| HSbrain_809_r | 156.7134677 |
| HS_from_MQbrain_192_l | 156.563744 |
| HSbrain_698_l | 156.4505714 |
| HSbrain_59_l | 156.0696115 |
| HS_from_MQbrain_178_l | 155.9319043 |
| HSbrain_663_r | 155.8547049 |
| HS_112_l | 155.7901438 |
| HS_from_MQbrain_135_l | 155.6010506 |
| HS_from_MQbrain_1445_l | 154.9673281 |
| HS_from_MQbrain_1304_r | 154.625417 |
| HS_from_MQbrain_1546_r | 154.5895228 |
| HS_from_MQbrain_1708_l | 154.1529962 |
| HS_from_MQbrain_1332_r | 154.1525563 |
| HS_from_MQbrain_92_l | 153.9678306 |
| HS_169_l | 153.6041594 |
| HS_from_MQbrain_1300_l | 153.3945718 |
| HSbrain_433_l | 153.3062571 |
| HSbrain_169_l | 153.1622142 |
| HS_from_MQbrain_1656_r | 153.0682586 |
| HS_from_MQbrain_1462_r | 152.9071604 |
| Hsd_73_r | 152.8453072 |
| HS_from_MQbrain_24_r | 152.6756691 |
| HS_from_MQbrain_501_l | 152.6321911 |
| HS_from_MQbrain_327_r | 151.8340882 |
| HS_from_MM_58_r | 151.5998526 |
| HS_from_PT_148_l | 151.4484249 |
| HS_from_MQbrain_317_l | 151.2288813 |
| HS_from_MM_155_r | 150.7239817 |
| HS_from_MQbrain_536_r | 150.565992 |
| Hsd_from_Mmd_148_l | 150.4939664 |
| HS_from_MM_349_l | 149.9283767 |
| HS_from_MM_203_l | 149.6945717 |
| HS_from_MQbrain_64_r | 149.6181093 |
| HSbrain_415_l | 148.8998605 |
| HS_from_MM_301_l | 148.8813987 |
| HS_from_MQbrain_661_l | 148.8113189 |
| HSbrain_1399_l | 148.5693182 |
| HSbrain_436_l | 148.4603877 |
| HSbrain_734_r | 147.8679536 |
| HS_from_MQbrain_1751_r | 147.8048267 |
| HS_from_MQbrain_1835_r | 147.7399163 |
| HS_from_MQbrain_1547_l | 147.2749783 |
| HS_22_l | 147.2714514 |
| HS_from_MQbrain_1637_r | 147.2561547 |
| Hsd_from_Mmd_229_l | 147.2551129 |
| Hsd_from_Mmd_235_r | 145.886021 |
| HS_from_PT_340_r | 145.5808035 |
| HSbrain_353_r | 145.5612086 |
| HS_from_MQbrain_486_l | 145.4138995 |
| Hsd_47_l | 145.2279529 |
| HS_from_MQbrain_117_l | 145.1372697 |
| HS_from_MQbrain_1744_r | 145.1169393 |
| HS_from_MQbrain_118_l | 145.0981054 |
| HSbrain_1305_l | 145.0316211 |
| HS_from_MQbrain_500_l | 144.96318 |
| HS_81_l | 144.8341455 |
| Hsd_from_Mmd_326_r | 144.7386621 |
| HS_from_MQbrain_585_r | 144.4245525 |
| HS_from_PT_61_l | 144.2294271 |
| HS_from_MQbrain_1231_r | 144.0677966 |
| HSbrain_557_r | 143.8626534 |
| HS_from_MQbrain_143_r | 143.5053792 |
| HSbrain_251_l | 143.3515241 |
| Hsd_from_Mmd_49_r | 143.2298967 |
| HS_from_MQbrain_841_l | 143.150258 |
| Hsd_from_Mmd_515_l | 143.0976097 |
| HS_from_MM_310_r | 143.0476245 |
| HS_from_MQbrain_287_r | 143.0394094 |
| HS_from_MQbrain_1555_l | 143.0040258 |
| HS_from_PT_4_r | 142.9952763 |
| HS_from_MQbrain_1209_r | 142.9625798 |
| HSbrain_473_r | 142.4884603 |
| HS_from_MQbrain_1749_r | 142.2592046 |
| HS_from_MM_303_l | 142.2483555 |
| Hsd_89_r | 142.2147592 |
| HS_from_MQbrain_1722_r | 141.984292 |
| HSbrain_721_l | 141.8888402 |
| HS_141_l | 141.8565157 |
| HS_from_MM_120_l | 141.8371708 |
| HS_from_MQbrain_663_r | 141.7046002 |
| Hsd_24_l | 141.5777402 |
| Hsd_67_l | 141.5629903 |
| HS_from_MQbrain_1540_l | 141.3634303 |
| HS_from_PT_180_l | 141.1839728 |
| HS_from_MM_136_r | 140.3205599 |
| HS_53_r | 140.0976088 |
| HSbrain_568_l | 139.1797861 |
| HSbrain_253_r | 138.9562066 |
| HS_from_PT_190_r | 138.4453126 |
| HS_from_MQbrain_1108_r | 138.2280125 |
| HS_20_r | 137.9175851 |
| HS_67_l | 137.6760885 |
| HSbrain_423_l | 137.5975104 |
| HS_from_MQbrain_494_l | 137.4594214 |
| HS_from_MQbrain_295_l | 137.1824078 |
| HS_203_r | 137.1520157 |
| HS_from_MQbrain_1809_r | 137.1399241 |
| HS_from_MM_160_l | 137.0116145 |
| Hsd_from_Mmd_439_r | 136.90383 |
| HS_from_MQbrain_1143_r | 136.8939224 |
| Hsd_from_Mmd_145_l | 136.8745882 |
| HSbrain_760_l | 136.864711 |
| HS_from_MQbrain_81_r | 136.5784067 |
| HSbrain_655_r | 136.4458282 |
| HS_from_MQbrain_1213_l | 136.145634 |
| HS_from_MQbrain_264_r | 136.066419 |
| HSbrain_727_l | 136.0474595 |
| HS_from_MQbrain_1790_r | 136.0259392 |
| HS_from_MQbrain_1658_r | 135.9727881 |
| HSbrain_790_r | 135.8835342 |
| HS_from_MQbrain_1858_r | 135.4615782 |
| HS_from_MM_33_l | 135.3503595 |
| HS_from_MQbrain_1739_r | 135.304321 |
| HS_from_MQbrain_349_l | 135.3020631 |
| HSbrain_77_r | 135.0441482 |
| Hsd_52_l | 134.5688394 |
| HS_from_MQbrain_899_r | 134.2391978 |
| HSbrain_230_r | 134.2244602 |
| HS_from_MQbrain_100_l | 133.6513061 |
| HS_from_MQbrain_1558_r | 133.4364824 |
| HS_from_MQbrain_1886_r | 133.3451731 |
| HS_from_MM_326_l | 133.0145352 |
| HS_from_MQbrain_632_l | 132.6729821 |
| HSbrain_177_r | 132.197755 |
| HSbrain_197_l | 132.1670897 |
| HS_from_MQbrain_34_r | 132.0668399 |
| HS_from_MM_74_l | 131.7543823 |
| HSbrain_811_r | 131.6313177 |
| HSbrain_286_l | 131.389336 |
| HS_251_l | 131.2661176 |
| HS_from_MQbrain_848_r | 131.2552614 |
| HS_from_MQbrain_470_l | 131.1632076 |
| HS_from_MQbrain_946_l | 131.0628786 |
| HS_from_MM_163_r | 130.6907731 |
| Hsd_44_l | 130.5671893 |
| Hsd_13_l | 130.4556358 |
| HSbrain_787_r | 129.6934291 |
| HS_from_MQbrain_1560_l | 129.614049 |
| HS_from_PT_269_l | 129.2301469 |
| HS_from_MM_122_r | 129.1183074 |
| HS_from_MQbrain_1513_r | 128.9407151 |
| HS_267_r | 128.8526 |
| HS_from_MQbrain_1610_r | 128.7979365 |
| HS_184_l | 128.6974233 |
| HS_from_MQbrain_1097_r | 128.6204149 |
| HS_from_PT_187_l | 128.3298072 |
| HS_from_MM_184_l | 128.1210167 |
| HS_from_MQbrain_151_r | 128.117498 |
| HSbrain_773_l | 127.1133192 |
| HS_from_MQbrain_1875_l | 126.9680493 |
| HS_from_MQbrain_1350_r | 126.8032255 |
| Hsd_from_Mmd_527_r | 126.3935973 |
| HS_110_l | 126.3504225 |
| HSbrain_730_l | 126.2182555 |
| HS_from_MQbrain_772_r | 126.0000486 |
| HS_from_MQbrain_296_r | 125.6165669 |
| HS_from_MQbrain_1517_l | 125.3704261 |
| HS_282_l | 125.3637264 |
| HS_from_MQbrain_842_r | 125.3022173 |
| HS_from_MQbrain_1788_r | 125.1671429 |
| Hsd_from_Mmd_482_r | 124.9021202 |
| HS_from_MM_317_l | 124.2047934 |
| HS_from_MQbrain_253_l | 124.158129 |
| HSbrain_585_l | 124.1437027 |
| HS_from_MM_124_r | 124.074717 |
| HSbrain_491_r | 123.9139471 |
| Hsd_from_Mmd_211_r | 123.6363171 |
| HS_from_MQbrain_1497_r | 123.458136 |
| HS_from_MQbrain_564_r | 123.2670175 |
| Hsd_from_Mmd_333_r | 122.8958947 |
| HS_from_MQbrain_1896_l | 122.7981574 |
| HS_from_MQbrain_619_r | 122.6749499 |
| HSbrain_697_l | 122.5351278 |
| Hsd_from_Mmd_39_l | 122.3556843 |
| Hsd_27_l | 122.1520322 |
| HS_from_MQbrain_1292_r | 121.9448163 |
| HS_216_r | 121.8875355 |
| HS_from_MQbrain_1535_l | 121.7711186 |
| HS_162_l | 121.3153743 |
| HS_from_MQbrain_578_l | 121.0148576 |
| HS_from_MM_198_l | 120.6300187 |
| HS_232_l | 120.592551 |
| Hsd_from_Mmd_274_l | 120.5723322 |
| HS_63_l | 120.5119395 |
| HS_from_MQbrain_777_r | 120.2896834 |
| HS_from_MQbrain_1205_r | 120.077252 |
| Hsd_from_Mmd_367_l | 120.0545039 |
| HS_from_MM_359_r | 120.0491134 |
| HS_from_MQbrain_1895_l | 119.7422297 |
| HSbrain_443_r | 119.5321197 |
| HS_from_MQbrain_1487_r | 119.4385433 |
| HS_from_PT_333_l | 119.2922172 |
| HS_from_MM_98_r | 118.6872322 |
| HS_from_MQbrain_502_r | 118.3776111 |
| HS_from_PT_303_l | 118.2462387 |
| Hsd_from_Mmd_324_r | 118.2362331 |
| HSbrain_764_r | 117.9645256 |
| HS_189_l | 117.9592496 |
| HSbrain_297_r | 117.8391655 |
| HSbrain_763_r | 117.7544498 |
| HS_from_PT_205_r | 117.5534749 |
| Hsd_from_Mmd_328_r | 117.324613 |
| HS_from_MQbrain_579_r | 117.2839857 |
| HSbrain_40_r | 117.0487953 |
| HSbrain_574_r | 116.9214286 |
| HS_from_MQbrain_608_r | 116.612101 |
| Hsd_from_Mmd_178_r | 116.4898429 |
| HSbrain_505_l | 116.4569543 |
| HS_from_MQbrain_1801_r | 116.3921742 |
| HS_from_MQbrain_1657_r | 116.3148049 |
| Hsd_from_Mmd_505_r | 116.0776309 |
| HSbrain_411_l | 116.0440512 |
| HSbrain_173_r | 115.8303113 |
| HS_43_r | 115.809889 |
| HSbrain_58_l | 115.8018906 |
| Hsd_from_Mmd_509_l | 115.5595143 |
| HSbrain_339_l | 115.4702672 |
| HS_from_MQbrain_1662_l | 114.9208166 |
| Hsd_from_Mmd_81_l | 114.8866476 |
| HSbrain_605_r | 114.7989073 |
| Hsd_87_r | 114.6751896 |
| Hsd_from_Mmd_34_r | 114.2335916 |
| HS_from_MQbrain_300_r | 114.1433066 |
| HS_from_MQbrain_530_l | 113.6020847 |
| HSbrain_705_r | 113.5172383 |
| HS_from_MQbrain_874_r | 113.4583818 |
| HS_from_MQbrain_468_l | 113.3244619 |
| HS_from_PT_313_l | 113.037452 |
| HS_from_MQbrain_132_l | 112.7869161 |
| HS_41_r | 112.6985514 |
| HS_from_MQbrain_154_l | 112.5010282 |
| HSbrain_498_l | 112.3225684 |
| HS_from_MQbrain_1522_r | 112.3011721 |
| HSbrain_274_r | 112.2207143 |
| HS_231_r | 112.1781103 |
| HS_from_MQbrain_1765_r | 111.7808146 |
| Hsd_from_Mmd_161_l | 111.7055777 |
| HS_from_MQbrain_1207_l | 111.657586 |
| HSbrain_262_l | 111.641823 |
| HS_from_MQbrain_1377_l | 110.9676187 |
| HS_from_MQbrain_949_l | 110.9567782 |
| HSbrain_523_r | 110.6926143 |
| HSbrain_733_l | 110.4880393 |
| Hsd_from_Mmd_182_l | 110.3509492 |
| HS_from_MQbrain_1408_l | 110.2916582 |
| HSbrain_482_l | 110.0149117 |
| HS_from_MQbrain_194_l | 110.0069304 |
| HS_from_MQbrain_797_l | 109.7202558 |
| Hsd_from_Mmd_187_r | 109.5214584 |
| HS_from_MQbrain_790_r | 109.3000205 |
| HS_from_MQbrain_687_r | 109.1779088 |
| HSbrain_563_l | 109.1569505 |
| HS_from_MQbrain_1208_l | 108.8734707 |
| HS_from_MQbrain_1503_r | 108.6188834 |
| HS_from_MQbrain_517_r | 108.5936311 |
| HS_from_MM_28_l | 108.5425494 |
| HSbrain_263_r | 108.4481826 |
| HS_from_MQbrain_382_r | 108.3108904 |
| HS_254_r | 108.0512602 |
| HS_from_MQbrain_361_l | 108.0467095 |
| HS_from_MQbrain_1072_r | 107.9850118 |
| HS_from_MQbrain_1769_r | 107.9535881 |
| Hsd_60_l | 107.914618 |
| HS_from_MM_93_l | 107.7887026 |
| HS_from_MM_210_r | 107.730886 |
| HSbrain_471_l | 107.7098195 |
| HS_from_MQbrain_804_l | 107.584966 |
| HS_from_MQbrain_593_r | 107.4432597 |
| HS_from_MQbrain_1457_l | 107.3708718 |
| HS_from_PT_74_r | 107.2883837 |
| Hsd_from_Mmd_481_l | 107.0549259 |
| Hsd_from_Mmd_69_l | 106.5131657 |
| Hsd_from_Mmd_195_r | 106.3658909 |
| HS_from_MM_294_l | 106.3355438 |
| HSbrain_272_r | 106.1087732 |
| HS_from_MQbrain_1169_l | 105.7095098 |
| HSbrain_9_l | 105.6311858 |
| Hsd_from_Mmd_271_r | 105.5217433 |
| HS_from_MQbrain_606_r | 105.4640055 |
| HS_from_MQbrain_1466_r | 105.3355825 |
| HS_from_MQbrain_575_r | 105.2312305 |
| HS_from_MQbrain_628_l | 105.1976832 |
| HS_from_MQbrain_113_l | 105.1838551 |
| Hsd_from_Mmd_136_r | 104.7410844 |
| HSbrain_128_r | 104.3537561 |
| HS_from_MM_314_l | 103.7363449 |
| HSbrain_336_l | 103.7156038 |
| HS_from_MQbrain_1793_r | 103.5900968 |
| HS_from_MQbrain_244_l | 102.9921795 |
| HSbrain_782_r | 102.9919045 |
| HS_from_PT_291_l | 102.8033399 |
| HSbrain_466_l | 102.7199119 |
| HS_from_PT_182_r | 102.5770161 |
| HS_213_r | 102.5631518 |
| HS_from_MQbrain_1892_l | 102.5141646 |
| HSbrain_754_l | 102.2895853 |
| HS_from_MQbrain_537_l | 101.9504176 |
| HS_from_MQbrain_1037_r | 101.466981 |
| Hsd_35_r | 101.1446856 |
| HS_from_PT_1_l | 101.0084568 |
| HSbrain_117_r | 100.8429431 |
| HS_from_MM_109_r | 100.8350177 |
| HSbrain_1400_r | 100.7941952 |
| HSbrain_113_r | 100.6056081 |
| HSbrain_157_r | 100.3340918 |
| Hsd_from_Mmd_534_l | 100.2946832 |
| HSbrain_747_l | 100.2134098 |
| HS_from_MQbrain_509_r | 100.1137811 |
| HS_from_MQbrain_713_r | 99.8596546 |
| HSbrain_653_r | 99.76505351 |
| HS_from_MM_193_l | 99.7576611 |
| HS_from_MQbrain_622_r | 99.6479399 |
| HS_from_MM_158_l | 99.547531 |
| HSbrain_14_r | 99.51998497 |
| HSbrain_82_r | 99.35128086 |
| HS_from_MQbrain_293_l | 99.25916842 |
| HS_from_MQbrain_110_r | 98.94375 |
| HS_from_MQbrain_1851_r | 98.90373913 |
| HS_from_MQbrain_799_l | 98.83429895 |
| HSbrain_418_l | 98.58964973 |
| HS_from_MM_287_l | 98.35019506 |
| HSbrain_239_l | 98.26806599 |
| HSbrain_42_l | 97.38728405 |
| Hsd_16_l | 97.34868547 |
| Hsd_from_Mmd_197_r | 97.00385398 |
| HS_85_l | 96.48110294 |
| HSbrain_94_l | 96.33702157 |
| HSbrain_294_l | 96.30247326 |
| Hsd_from_Mmd_485_r | 95.1476108 |
| Hsd_from_Mmd_156_r | 94.89926352 |
| HS_from_MQbrain_1034_r | 94.58788064 |
| HSbrain_624_l | 94.53967575 |
| HS_from_MQbrain_1326_r | 94.53541538 |
| HS_from_MQbrain_91_l | 94.2325 |
| Hsd_from_Mmd_410_l | 94.2059708 |
| HS_from_MQbrain_112_l | 94.04683229 |
| Hsd_86_l | 93.94420635 |
| HS_from_MQbrain_1646_r | 93.89323261 |
| HS_from_MQbrain_839_r | 92.94488973 |
| HS_from_PT_136_l | 92.74159284 |
| HS_177_r | 92.49572239 |
| HS_from_PT_217_r | 92.06195569 |
| HSbrain_1384_l | 91.87184624 |
| HS_from_MQbrain_355_r | 91.78580467 |
| HS_36_l | 91.72099648 |
| HSbrain_633_r | 91.40733696 |
| Hsd_from_Mmd_436_r | 90.76582879 |
| HS_from_MQbrain_997_l | 90.55621189 |
| HS_from_MQbrain_1485_r | 90.24185865 |
| Hsd_from_Mmd_76_r | 90.16068804 |
| HS_from_MM_187_l | 90.14742716 |
| HS_from_MQbrain_1703_l | 89.75751515 |
| HS_from_MM_86_l | 89.48891073 |
| HSbrain_97_l | 89.025924 |
| HS_from_MQbrain_1958_r | 88.93893093 |
| HS_from_MQbrain_2670_r | 88.75898562 |
| HSbrain_427_r | 88.68101495 |
| HS_from_MQbrain_96_r | 88.6327113 |
| Hsd_from_Mmd_74_r | 88.62981818 |
| HS_from_MQbrain_769_r | 87.406602 |
| HSbrain_195_l | 87.10754423 |
| HS_from_MQbrain_792_l | 87.00610998 |
| HS_from_MQbrain_1334_l | 86.86195926 |
| HS_from_MM_178_r | 86.39079121 |
| HS_from_MQbrain_315_l | 85.77155971 |
| HS_from_PT_139_l | 85.75830736 |
| HSbrain_337_l | 85.40127884 |
| Hsd_from_Mmd_388_r | 85.25768575 |
| HS_from_MQbrain_1290_l | 84.29078309 |
| HS_from_MQbrain_261_r | 84.17408031 |
| HS_from_MQbrain_1197_r | 83.62885897 |
| HS_158_r | 83.42896638 |
| HSbrain_261_l | 83.18937251 |
| HS_from_MQbrain_1527_l | 83.08155754 |
| HS_from_MQbrain_212_r | 83.02395006 |
| HS_from_MQbrain_15_l | 82.91376316 |
| HS_from_MM_135_r | 82.76328898 |
| Hsd_from_Mmd_90_r | 82.48617419 |
| HS_from_MQbrain_1779_l | 82.35801621 |
| HSbrain_343_l | 82.34298795 |
| HS_193_l | 82.06496463 |
| HS_from_MQbrain_923_l | 81.9527027 |
| HSbrain_224_r | 81.77304743 |
| HSbrain_506_l | 81.68257158 |
| HS_from_PT_251_r | 81.54576842 |
| HS_from_MQbrain_1791_l | 81.49102222 |
| Hsd_76_r | 80.46787511 |
| HS_from_MM_40_l | 80.43791933 |
| Hsd_from_Mmd_525_r | 80.02408705 |
| HS_from_MQbrain_952_l | 79.24788235 |
| Hsd_from_Mmd_343_l | 79.17022973 |
| HS_from_MQbrain_1139_l | 79.05020237 |
| HS_from_MM_285_r | 79.00004832 |
| HS_from_MQbrain_832_l | 78.79201419 |
| HS_from_MM_260_r | 78.59914649 |
| Hsd_from_Mmd_455_r | 78.59609185 |
| HS_from_MQbrain_483_r | 78.46549193 |
| HS_from_MQbrain_506_l | 78.43916484 |
| HS_from_MM_106_r | 78.0376375 |
| HSbrain_329_l | 77.87290923 |
| HS_68_l | 77.85150167 |
| HS_from_MQbrain_240_l | 77.63282587 |
| HSbrain_254_l | 77.56906513 |
| HSbrain_458_l | 77.3089971 |
| HSbrain_358_l | 77.25165627 |
| HSbrain_1_l | 76.8373873 |
| HS_from_PT_284_l | 76.78239533 |
| HS_from_MM_13_r | 76.61267549 |
| HS_from_MM_149_r | 75.91523091 |
| HS_from_MQbrain_1525_l | 75.43714328 |
| HSbrain_106_r | 74.6228739 |
| HS_from_MQbrain_1089_r | 74.25842215 |
| HS_from_PT_103_l | 74.08268376 |
| Hsd_from_Mmd_296_r | 74.00030271 |
| HS_from_MQbrain_1092_l | 73.98551161 |
| HS_from_MQbrain_820_l | 73.70641218 |
| HS_from_MQbrain_1760_l | 73.58132161 |
| HS_from_MQbrain_141_l | 73.5043697 |
| HS_from_MQbrain_1206_r | 73.21400029 |
| HSbrain_648_r | 73.09661808 |
| HS_from_MQbrain_525_r | 72.8823751 |
| Hsd_from_Mmd_476_l | 72.68804749 |
| HS_from_MQbrain_991_r | 72.5119085 |
| HS_from_MQbrain_1354_l | 72.49188083 |
| HSbrain_582_r | 72.28969575 |
| HS_from_MQbrain_641_r | 72.1752804 |
| HS_from_MQbrain_1642_r | 71.93220437 |
| HS_from_MQbrain_1772_r | 71.75524419 |
| HS_from_MQbrain_1056_l | 71.54390765 |
| HSbrain_1436_r | 71.34269518 |
| HSbrain_278_r | 71.09610068 |
| HSbrain_707_r | 70.97237044 |
| HS_from_MM_118_r | 70.86890311 |
| HSbrain_13_r | 70.64600087 |
| HS_from_MQbrain_304_r | 70.56355992 |
| HSbrain_1391_l | 70.35762568 |
| HS_from_MQbrain_1080_l | 70.18685041 |
| HSbrain_553_l | 69.73500772 |
| HS_from_MQbrain_224_l | 69.6781412 |
| HS_from_PT_157_l | 69.35695138 |
| HS_from_MQbrain_1600_r | 69.30857587 |
| HS_from_MQbrain_915_l | 68.77873899 |
| HS_from_MM_185_r | 68.74396066 |
| HS_from_MM_119_l | 68.49168012 |
| HS_from_MQbrain_1410_r | 68.45911099 |
| HSbrain_25_r | 68.45071483 |
| HSbrain_689_l | 68.40960966 |
| HS_from_MQbrain_68_l | 68.40129245 |
| HSbrain_1337_r | 68.28435609 |
| HS_172_r | 66.96978425 |
| Hsd_92_l | 66.70992407 |
| HS_from_MQbrain_1396_l | 66.69299102 |
| HSbrain_1417_r | 66.48280656 |
| HS_62_l | 66.38174565 |
| HSbrain_1410_l | 66.2043953 |
| HS_59_l | 66.17528144 |
| HSbrain_672_l | 65.33492962 |
| HS_96_l | 65.1735359 |
| HS_from_MQbrain_255_l | 64.86378183 |
| HSbrain_1387_l | 64.82240984 |
| HSbrain_572_r | 64.68912173 |
| HS_from_PT_328_l | 64.37288073 |
| HSbrain_756_l | 64.23391136 |
| Hsd_from_Mmd_532_l | 63.98178984 |
| HS_from_MQbrain_2050_l | 63.61829063 |
| HS_from_MQbrain_465_l | 63.48237355 |
| HS_from_MQbrain_235_l | 63.18667666 |
| HSbrain_178_l | 63.1279546 |
| HS_from_MQbrain_835_r | 62.94556533 |
| HSbrain_429_r | 62.78468363 |
| HS_from_MQbrain_1109_l | 62.73503843 |
| Hsd_from_Mmd_461_l | 62.47532833 |
| HS_from_MQbrain_80_l | 62.18301452 |
| Hsd_19_l | 62.00218054 |
| Hsd_from_Mmd_68_l | 61.78328125 |
| HS_from_MQbrain_1048_r | 61.35298515 |
| HS_from_MQbrain_838_l | 60.49841307 |
| HSbrain_295_r | 60.35523089 |
| HS_from_MQbrain_1250_r | 60.18097983 |
| HS_from_PT_320_l | 60.05463755 |
| HS_from_PT_196_r | 59.89771429 |
| HS_from_MQbrain_1314_r | 59.71438986 |
| HSbrain_617_l | 59.52352443 |
| HSbrain_529_l | 59.40625213 |
| Hsd_from_Mmd_170_r | 59.3789276 |
| HSbrain_700_r | 59.35494614 |
| HSbrain_666_l | 58.90254095 |
| HSbrain_670_l | 58.71863975 |
| HS_from_MM_159_r | 58.25485462 |
| HS_from_MQbrain_625_l | 58.16380903 |
| HSbrain_170_r | 58.09549284 |
| HSbrain_736_r | 57.98906648 |
| HSbrain_512_r | 57.96648247 |
| HS_from_MQbrain_1603_r | 57.86596708 |
| HSbrain_604_r | 57.67387084 |
| HS_from_MQbrain_193_r | 57.49416629 |
| HSbrain_686_l | 57.37727461 |
| HS_from_MQbrain_800_r | 57.33831283 |
| HS_from_MM_194_r | 57.30809246 |
| HS_from_MQbrain_1734_l | 57.12642239 |
| HS_from_MQbrain_453_l | 57.09533664 |
| HSbrain_806_l | 56.98000317 |
| Hsd_3_l | 56.90308663 |
| HS_from_MQbrain_759_r | 56.84789382 |
| Hsd_from_Mmd_340_r | 56.81841128 |
| HS_from_MQbrain_1058_r | 56.74296423 |
| HSbrain_354_r | 56.5121469 |
| HSbrain_89_l | 56.40365692 |
| HSbrain_1398_l | 56.38379162 |
| Hsd_40_r | 56.20399334 |
| Hsd_51_r | 56.05310726 |
| HS_276_r | 56.05146797 |
| HS_from_MQbrain_808_r | 55.94239194 |
| HSbrain_200_l | 55.88350406 |
| HS_from_MM_114_r | 55.53559367 |
| HSbrain_405_l | 55.37192181 |
| HSbrain_1388_l | 55.06423423 |
| HS_from_MQbrain_809_r | 54.98604134 |
| HS_from_MQbrain_1378_l | 54.95023303 |
| HS_from_MQbrain_972_l | 54.8749493 |
| HSbrain_380_r | 54.63237608 |
| HS_from_MQbrain_833_r | 54.62093985 |
| HS_from_MQbrain_907_r | 54.46335077 |
| HS_from_MQbrain_1815_r | 54.40047492 |
| HS_from_MQbrain_791_l | 54.34606891 |
| HSbrain_333_l | 54.27871271 |
| Hsd_from_Mmd_358_l | 54.15061703 |
| HS_from_MQbrain_1502_l | 54.14110189 |
| HSbrain_678_l | 54.10203966 |
| HSbrain_12_l | 53.76557022 |
| HSbrain_449_l | 53.67127059 |
| Hsd_88_l | 53.39128816 |
| HSbrain_737_r | 53.36757802 |
| HS_from_MQbrain_1248_r | 53.19833147 |
| Hsd_6_l | 53.15042029 |
| Hsd_from_Mmd_507_r | 52.61515894 |
| HSbrain_758_l | 52.56138123 |
| HS_from_MQbrain_676_r | 52.19716118 |
| HS_from_MQbrain_1782_l | 52.131107 |
| Hsd_from_Mmd_219_l | 51.9531264 |
| Hsd_from_Mmd_222_r | 51.80246158 |
| Hsd_from_Mmd_159_l | 51.66519936 |
| Hsd_from_Mmd_263_r | 51.62052609 |
| HS_from_MQbrain_552_r | 51.35410703 |
| HSbrain_3_l | 51.24508411 |
| HS_100_l | 51.22672886 |
| HSbrain_131_l | 51.07910404 |
| HSbrain_1390_l | 50.92997516 |
| HS_214_l | 50.60281362 |
| HS_from_MM_201_l | 50.5621644 |
| HSbrain_314_r | 50.36125959 |
| HS_69_l | 50.17481884 |
| HSbrain_674_l | 50.10105834 |
| HS_from_MQbrain_435_l | 50.08067692 |
| HS_from_MQbrain_910_l | 50.04935128 |
| HSbrain_1389_l | 49.94495038 |
| HSbrain_1392_l | 49.93511597 |
| HS_from_PT_32_l | 49.84882629 |
| HS_from_MQbrain_72_l | 49.84100507 |
| HSbrain_795_l | 49.81882127 |
| HSbrain_2_l | 49.67189807 |
| HS_from_MQbrain_153_l | 49.32700101 |
| HSbrain_530_l | 49.2660315 |
| HS_from_MQbrain_1628_l | 49.23189915 |
| HS_from_MQbrain_478_r | 49.18860716 |
| HS_from_MQbrain_353_r | 49.02897584 |
| HS_from_PT_130_r | 49.02454902 |
| HS_187_r | 48.74477955 |
| HSbrain_163_r | 48.59342229 |
| HSbrain_1386_l | 48.57929064 |
| HSbrain_667_l | 48.3302073 |
| Hsd_from_Mmd_298_r | 48.27315482 |
| HSbrain_683_l | 48.21895122 |
| HS_from_MQbrain_79_r | 48.19976833 |
| HSbrain_236_r | 47.93509812 |
| HS_from_MQbrain_970_l | 47.65771607 |
| HS_from_MM_219_l | 47.65091531 |
| HSbrain_662_r | 47.55963248 |
| HS_from_MQbrain_864_l | 47.52132335 |
| HS_from_MQbrain_701_l | 47.39067123 |
| HS_from_MQbrain_906_r | 47.10729944 |
| HS_252_l | 46.90321783 |
| HSbrain_127_l | 46.52896356 |
| HSbrain_246_r | 46.43534933 |
| HSbrain_681_l | 46.40728223 |
| Hsd_from_Mmd_280_l | 46.29319877 |
| HSbrain_660_l | 45.8415198 |
| HSbrain_370_r | 45.65991936 |
| HSbrain_285_l | 45.49193279 |
| HSbrain_504_r | 45.47207589 |
| HSbrain_704_l | 45.12744924 |
| HS_from_PT_48_r | 44.70075948 |
| HS_from_MQbrain_1638_l | 44.5951134 |
| HS_from_MQbrain_1823_r | 44.43291919 |
| HS_from_MQbrain_227_l | 44.22949556 |
| HS_from_MQbrain_168_l | 44.17804845 |
| HSbrain_344_l | 44.15992199 |
| HS_from_MQbrain_526_r | 44.11167126 |
| HS_from_MM_96_l | 44.0116193 |
| HS_from_MQbrain_895_l | 43.98720722 |
| HS_from_MQbrain_1671_r | 43.89097046 |
| HS_8_l | 43.81193213 |
| HSbrain_1434_r | 43.6359673 |
| Hsd_from_Mmd_391_l | 43.63503635 |
| HS_239_r | 43.51954011 |
| HSbrain_119_l | 43.4608124 |
| HS_from_MM_313_l | 43.35911147 |
| HSbrain_46_r | 42.59001781 |
| HS_from_MQbrain_912_l | 42.57025194 |
| HSbrain_332_l | 42.50762875 |
| HSbrain_492_r | 42.32362121 |
| HS_from_MQbrain_1595_l | 42.27660177 |
| HS_from_MM_1_r | 42.18437325 |
| HS_from_MQbrain_1461_l | 42.18381426 |
| HS_from_MM_298_l | 41.95783366 |
| HSbrain_753_r | 41.77257724 |
| HS_from_PT_140_r | 41.76036155 |
| HSbrain_447_r | 41.25875347 |
| HS_from_MQbrain_1723_l | 41.19154968 |
| HS_from_MQbrain_634_r | 40.90596624 |
| HS_from_MQbrain_195_l | 40.82226933 |
| HS_from_MQbrain_451_l | 39.84713568 |
| HS_from_MQbrain_816_l | 39.60354675 |
| HS_from_MM_146_l | 39.24758442 |
| HSbrain_645_r | 39.08920999 |
| HS_from_PT_254_r | 38.90953686 |
| HS_from_MQbrain_1439_l | 38.7147919 |
| HSbrain_577_r | 38.5784199 |
| HSbrain_142_l | 38.55033479 |
| HS_from_MQbrain_1872_l | 38.54135356 |
| HS_147_l | 38.35905769 |
| HS_from_MQbrain_334_l | 37.94381448 |
| HSbrain_789_r | 37.88985765 |
| Hsd_from_Mmd_533_l | 37.74138535 |
| HS_from_MM_168_l | 37.71479168 |
| HS_153_r | 37.62053177 |
| HS_from_MQbrain_778_r | 37.2177956 |
| HSbrain_412_l | 37.17793298 |
| HSbrain_603_l | 37.11164773 |
| HS_from_MQbrain_1168_r | 37.02284436 |
| HSbrain_235_r | 36.73133147 |
| HSbrain_378_l | 36.4906662 |
| HSbrain_180_r | 36.33126606 |
| HS_from_MQbrain_984_l | 36.17853558 |
| HSbrain_619_l | 35.93463694 |
| HS_174_l | 35.20952323 |
| HS_from_MQbrain_573_l | 34.88927084 |
| Hsd_from_Mmd_231_l | 34.74078503 |
| HSbrain_338_l | 34.72414818 |
| Hsd_from_Mmd_355_l | 34.42795833 |
| Hsd_from_Mmd_41_r | 34.27587083 |
| Hsd_from_Mmd_486_r | 34.15274704 |
| HS_from_MQbrain_549_l | 34.06783333 |
| HS_from_MQbrain_1269_l | 33.96400251 |
| HSbrain_382_l | 33.54888194 |
| HS_from_MQbrain_1296_l | 33.13683698 |
| Hsd_33_l | 33.05107162 |
| Hsd_59_r | 33.04438013 |
| HS_103_r | 32.96905528 |
| HS_from_MQbrain_339_l | 32.88359006 |
| HS_from_MM_101_l | 32.5944636 |
| HS_from_MQbrain_1541_l | 32.58806331 |
| HS_from_MQbrain_594_l | 32.57661566 |
| HS_from_MQbrain_441_l | 32.28225695 |
| HS_from_MQbrain_1180_r | 32.20670401 |
| Hsd_from_Mmd_311_l | 32.03234884 |
| Hsd_from_Mmd_51_l | 31.73933766 |
| HS_from_MQbrain_945_l | 31.71875825 |
| HSbrain_407_l | 31.47421874 |
| HS_from_MQbrain_1728_r | 31.38135841 |
| HS_from_MM_63_l | 31.12592861 |
| HS_from_MQbrain_476_r | 30.32765965 |
| Hsd_22_l | 30.26306383 |
| Hsd_from_Mmd_190_l | 30.1853939 |
| HSbrain_481_r | 29.96985078 |
| HS_from_MQbrain_813_l | 29.85107308 |
| HSbrain_81_l | 29.82507402 |
| HS_from_MQbrain_760_r | 29.64730363 |
| HS_from_MQbrain_836_l | 29.14988914 |
| HS_from_MM_116_r | 29.14631328 |
| HS_from_MQbrain_1405_l | 28.83096971 |
| Hsd_from_Mmd_536_r | 28.65654394 |
| HS_from_MQbrain_1528_l | 28.63818767 |
| HSbrain_62_l | 28.38621466 |
| HS_from_MQbrain_1469_r | 28.13492378 |
| HS_from_MM_147_l | 27.93559808 |
| HS_from_MQbrain_811_l | 27.92339435 |
| HS_from_PT_257_r | 27.728139 |
| Hsd_from_Mmd_59_l | 27.70423359 |
| HS_from_MQbrain_1147_l | 27.67089026 |
| HS_from_MQbrain_1531_r | 27.40554145 |
| HS_from_MQbrain_1404_r | 27.3954386 |
| HSbrain_1419_l | 27.03021687 |
| HS_from_MQbrain_187_l | 26.83013233 |
| Hsd_98_r | 26.78596273 |
| HS_from_MQbrain_358_r | 26.721815 |
| HS_from_MQbrain_1268_r | 26.46384108 |
| HS_from_MQbrain_444_l | 26.41588909 |
| HS_from_MQbrain_1384_r | 26.20937179 |
| HSbrain_543_r | 26.09736904 |
| HS_from_MQbrain_484_r | 25.86881457 |
| HS_from_MQbrain_377_r | 25.55774768 |
| Hsd_from_Mmd_426_l | 25.5337463 |
| HSbrain_422_r | 25.49910218 |
| HS_from_MM_89_r | 25.15500847 |
| HS_from_MQbrain_1301_l | 25.12455997 |
| HS_from_MQbrain_717_r | 25.09002247 |
| HS_from_MQbrain_1762_r | 24.82824464 |
| Hsd_from_Mmd_478_l | 24.52473283 |
| HSbrain_561_r | 24.46565743 |
| HS_from_MQbrain_829_l | 24.39741191 |
| HS_from_MM_90_r | 24.1951004 |
| HS_from_MQbrain_986_r | 24.18087433 |
| HS_from_MQbrain_1201_r | 23.9141836 |
| HS_from_MQbrain_157_r | 23.7389583 |
| HS_from_MQbrain_1571_l | 23.40380682 |
| Hsd_7_r | 23.39907905 |
| Hsd_104_l | 23.30618615 |
| HS_from_MQbrain_854_l | 23.28901375 |
| Hsd_from_Mmd_275_r | 23.18305355 |
| HS_from_MQbrain_979_l | 23.09100891 |
| HSbrain_417_l | 23.07219376 |
| HS_from_MQbrain_1320_l | 22.94039627 |
| Hsd_from_Mmd_389_r | 22.408902 |
| HSbrain_497_l | 22.40769173 |
| HS_from_MQbrain_1312_l | 22.37619915 |
| HSbrain_671_r | 22.1043858 |
| HSbrain_281_l | 21.97857516 |
| Hsd_from_Mmd_452_l | 21.7681635 |
| HS_from_MM_276_l | 21.75172107 |
| HS_from_MQbrain_402_r | 21.71995828 |
| HS_from_PT_272_l | 21.71160257 |
| HS_from_MQbrain_1690_l | 21.66605198 |
| HS_from_MQbrain_16_l | 21.18417377 |
| HS_from_MQbrain_812_r | 21.17953556 |
| HS_from_MQbrain_1883_l | 21.12907054 |
| HS_from_MQbrain_1081_r | 20.97667628 |
| HSbrain_848_l | 20.79326706 |
| HSbrain_237_r | 20.78891804 |
| Hsd_15_l | 20.57501838 |
| Hsd_from_Mmd_297_r | 20.55327833 |
| HSbrain_742_r | 20.55270084 |
| HS_from_MQbrain_1783_l | 20.23594438 |
| Hsd_from_Mmd_272_r | 19.9884149 |
| HS_from_MM_156_r | 19.97104349 |
| HSbrain_546_r | 19.96726522 |
| HS_from_PT_40_r | 19.96645961 |
| Hsd_from_Mmd_223_r | 19.94197059 |
| HS_from_MM_236_l | 19.92500437 |
| Hsd_from_Mmd_409_l | 19.84694165 |
| HSbrain_1422_l | 19.73606411 |
| HS_from_MQbrain_601_l | 19.43288221 |
| HSbrain_661_r | 19.37627406 |
| HSbrain_214_l | 19.36197308 |
| HSbrain_540_r | 19.28862768 |
| HSbrain_399_r | 19.20558511 |
| HSbrain_394_l | 19.15960956 |
| HSbrain_159_l | 19.09259883 |
| HSbrain_111_l | 19.09214846 |
| HS_from_MQbrain_954_l | 19.05644186 |
| HS_from_MQbrain_2441_l | 19.05254542 |
| HS_from_MQbrain_1234_l | 18.99466667 |
| HSbrain_302_l | 18.95338066 |
| HSbrain_654_l | 18.757945 |
| HS_from_MQbrain_137_l | 18.64970611 |
| HS_from_MQbrain_1606_r | 18.62823269 |
| HS_from_MQbrain_1327_r | 18.53143133 |
| HS_from_MQbrain_215_l | 18.49178278 |
| HS_from_MQbrain_462_r | 18.48192995 |
| HSbrain_1338_l | 18.40400866 |
| HS_from_MQbrain_1698_l | 18.29146104 |
| HS_from_PT_221_r | 18.28911853 |
| HS_from_MQbrain_1317_l | 18.27878358 |
| HS_from_MM_268_r | 18.2673582 |
| HS_from_MQbrain_1256_l | 18.09586294 |
| HSbrain_442_r | 18.08359556 |
| HS_from_MQbrain_1648_r | 18.04974603 |
| Hsd_26_r | 17.92523385 |
| HSbrain_225_l | 17.89446706 |
| HS_from_MQbrain_1308_r | 17.83505344 |
| HSbrain_238_l | 17.83400503 |
| Hsd_from_Mmd_149_r | 17.76480562 |
| HS_from_MQbrain_1474_l | 17.62620625 |
| HS_from_MQbrain_1286_l | 17.57104529 |
| HSbrain_379_r | 17.51053422 |
| HSbrain_542_l | 17.47601898 |
| HS_93_r | 17.4404153 |
| HS_from_MM_9_r | 17.31753403 |
| HS_from_MM_81_l | 17.28975439 |
| HS_from_MQbrain_471_r | 17.2822643 |
| HS_295_r | 17.2634514 |
| HS_272_r | 17.21224979 |
| HSbrain_381_r | 17.19500195 |
| Hsd_91_l | 17.19147953 |
| HSbrain_374_l | 17.14555494 |
| HS_from_PT_247_l | 17.14193784 |
| HS_from_MQbrain_1087_r | 17.09295407 |
| HSbrain_1406_l | 17.08291783 |
| HSbrain_499_r | 16.86242407 |
| HS_273_l | 16.84876947 |
| HS_from_MQbrain_105_l | 16.80746034 |
| HS_from_MQbrain_554_l | 16.7813907 |
| HS_145_l | 16.69161607 |
| HS_11_r | 16.56965243 |
| HS_from_MQbrain_1778_r | 16.56308131 |
| HS_from_MQbrain_1901_l | 16.55251009 |
| HS_298_r | 16.37083451 |
| HS_from_PT_125_l | 16.20117613 |
| HS_297_r | 16.18088267 |
| HSbrain_5_l | 16.1038857 |
| HS_from_MQbrain_814_r | 16.03485169 |
| HSbrain_357_l | 15.97090807 |
| HS_from_MQbrain_65_r | 15.88380756 |
| HS_13_l | 15.87695915 |
| HSbrain_649_l | 15.85119362 |
| HS_from_MQbrain_55_l | 15.80840103 |
| HS_260_l | 15.63834526 |
| Hsd_from_Mmd_262_l | 15.61807712 |
| HS_from_MM_39_l | 15.46864042 |
| HS_from_MQbrain_1882_r | 15.42159484 |
| HS_192_r | 15.41603426 |
| HS_from_PT_123_r | 15.33202181 |
| HS_from_PT_128_r | 15.33190969 |
| HSbrain_507_r | 15.19580403 |
| HSbrain_706_l | 15.17246512 |
| Hsd_from_Mmd_473_l | 15.1413037 |
| HS_from_MQbrain_1753_r | 15.00695265 |
| HS_from_MQbrain_420_r | 15.00394554 |
| HS_from_PT_292_l | 14.96523543 |
| HS_204_r | 14.93137289 |
| HSbrain_247_l | 14.87371333 |
| HSbrain_533_r | 14.81805007 |
| HSbrain_140_l | 14.80953804 |
| HS_from_MQbrain_1494_l | 14.7687191 |
| HSbrain_1423_r | 14.73538059 |
| Hsd_from_Mmd_524_r | 14.7018346 |
| HSbrain_602_r | 14.66781038 |
| HS_262_l | 14.64955868 |
| HS_from_MM_254_r | 14.60568724 |
| HS_from_MQbrain_1746_r | 14.49926674 |
| HS_from_MQbrain_1893_r | 14.47922582 |
| HSbrain_16_l | 14.42037004 |
| HS_from_MQbrain_1879_r | 14.39648047 |
| HSbrain_446_l | 14.33402718 |
| HSbrain_137_l | 14.29565616 |
| HS_294_r | 14.28845143 |
| HSbrain_356_l | 14.19629964 |
| HS_from_PT_155_r | 14.06410331 |
| HSbrain_24_l | 14.04788368 |
| HS_from_MQbrain_1811_l | 13.90556328 |
| HS_from_MQbrain_1799_l | 13.49508607 |
| HS_16_r | 13.45833857 |
| HS_from_PT_113_l | 13.41746343 |
| HS_from_MQbrain_1880_r | 13.37337073 |
| Hsd_9_l | 13.04883428 |
| HSbrain_732_r | 12.92048191 |
| HSbrain_432_l | 12.91311938 |
| HSbrain_454_r | 12.81394683 |
| Hsd_from_Mmd_249_r | 12.73986084 |
| HS_from_PT_195_l | 12.68428891 |
| HSbrain_256_r | 12.675 |
| HS_from_MM_72_l | 12.60592101 |
| HS_from_MQbrain_1195_l | 12.59863883 |
| HS_from_MQbrain_1188_r | 12.51525581 |
| HS_from_MQbrain_542_l | 12.4589606 |
| Hsd_from_Mmd_490_l | 12.34090149 |
| HS_202_l | 12.33606455 |
| Hsd_from_Mmd_325_l | 12.27846175 |
| HSbrain_641_l | 12.19531359 |
| HS_from_MQbrain_1805_r | 12.08133366 |
| HSbrain_222_l | 12.01722409 |
| HS_from_MQbrain_1214_l | 11.99962527 |
| HSbrain_486_r | 11.92687298 |
| HSbrain_190_l | 11.90155086 |
| HS_from_PT_282_l | 11.85767781 |
| HS_from_MQbrain_1857_l | 11.81637718 |
| HS_from_PT_253_l | 11.63356298 |
| HS_82_l | 11.58743783 |
| HS_from_MQbrain_992_l | 11.55219844 |
| HS_27_l | 11.5189217 |
| Hsd_55_l | 11.47708921 |
| HS_from_MQbrain_1217_l | 11.46181019 |
| HS_from_MQbrain_182_l | 11.36963928 |
| HS_from_PT_301_r | 11.35875076 |
| HS_from_PT_13_r | 11.28505999 |
| HS_from_PT_126_r | 11.24987161 |
| HS_from_MQbrain_1694_r | 11.24856812 |
| HSbrain_532_l | 11.20284123 |
| HS_from_PT_315_l | 11.14257669 |
| HS_from_MQbrain_656_l | 11.0380573 |
| HS_from_MQbrain_1264_r | 10.9879875 |
| HS_from_MQbrain_179_l | 10.93508111 |
| HSbrain_122_r | 10.91049034 |
| HSbrain_791_l | 10.8880558 |
| HSbrain_792_l | 10.74841217 |
| HS_from_MQbrain_8_r | 10.6772637 |
| HS_from_MQbrain_1432_l | 10.63790696 |
| Hsd_from_Mmd_523_l | 10.60175496 |
| HS_from_MQbrain_787_l | 10.45276823 |
| HSbrain_271_r | 10.39774564 |
| HS_from_PT_297_l | 10.3817856 |
| HS_from_MQbrain_389_l | 10.36562841 |
| Hsd_from_Mmd_306_r | 10.35597869 |
| HS_from_MQbrain_140_r | 10.33466558 |
| HS_130_l | 10.32443146 |
| Hsd_from_Mmd_336_r | 10.26104082 |
| Hsd_from_Mmd_243_r | 10.21164464 |
| HS_from_MQbrain_827_r | 10.04636996 |
| Hsd_from_Mmd_217_l | 10.0411297 |
| HS_from_MM_271_r | 9.974500263 |
| HSbrain_174_l | 9.914419841 |
| HS_from_MQbrain_1059_r | 9.903606705 |
| HSbrain_451_r | 9.891359358 |
| HS_from_MM_30_l | 9.828879468 |
| HSbrain_389_l | 9.742590244 |
| Hsd_from_Mmd_334_l | 9.461092816 |
| HSbrain_539_r | 9.402802755 |
| HS_from_MQbrain_172_r | 9.3950049 |
| Hsd_from_Mmd_491_r | 9.356107716 |
| HS_from_PT_321_r | 9.355284488 |
| HS_from_MM_214_r | 9.312015244 |
| HS_181_r | 9.247606951 |
| HSbrain_242_l | 9.140095581 |
| HSbrain_719_l | 9.057424006 |
| HS_from_PT_216_r | 9.04533293 |
| Hsd_106_l | 9.004764262 |
| HS_from_MQbrain_1216_l | 8.977281937 |
| HS_49_r | 8.969465473 |
| HS_from_MQbrain_521_r | 8.94922503 |
| HSbrain_76_r | 8.849660075 |
| HS_from_PT_309_l | 8.806106342 |
| HS_from_MM_275_r | 8.801848936 |
| HS_from_MQbrain_1784_r | 8.765325591 |
| HSbrain_320_l | 8.739860007 |
| HS_from_MM_169_r | 8.697594288 |
| HSbrain_414_r | 8.667025277 |
| HSbrain_468_r | 8.648929144 |
| HS_from_MQbrain_597_l | 8.606774472 |
| HS_from_MQbrain_1238_r | 8.560768136 |
| HS_from_MQbrain_1576_r | 8.509211792 |
| HSbrain_711_r | 8.507528601 |
| HS_270_l | 8.500056129 |
| HS_from_MQbrain_189_r | 8.458412909 |
| HSbrain_803_r | 8.436925155 |
| HS_from_MQbrain_354_l | 8.378373604 |
| HS_from_MQbrain_1514_r | 8.3514592 |
| Hsd_23_l | 8.323337473 |
| HS_from_MQbrain_329_r | 8.298733963 |
| HS_from_MQbrain_694_l | 8.295833858 |
| HS_from_PT_168_r | 8.28827405 |
| HS_from_MQbrain_209_l | 8.267384678 |
| HS_from_MQbrain_1124_l | 8.263702597 |
| HS_from_MQbrain_581_r | 8.223408593 |
| HS_from_MQbrain_796_r | 8.179623288 |
| Hsd_from_Mmd_464_r | 8.134582867 |
| HS_from_MQbrain_995_l | 8.045505431 |
| HSbrain_609_r | 8.027284091 |
| HS_296_l | 8.020342859 |
| HS_from_MM_164_r | 8.01189659 |
| Hsd_from_Mmd_422_r | 8.004189538 |
| HS_from_MM_34_l | 7.978839103 |
| Hsd_53_l | 7.976949043 |
| Hsd_from_Mmd_58_l | 7.966323875 |
| HS_from_MM_256_l | 7.965453015 |
| HSbrain_501_l | 7.947790019 |
| HS_from_MQbrain_817_l | 7.907551804 |
| HS_from_PT_138_r | 7.906213461 |
| HS_264_r | 7.905646906 |
| HS_from_MQbrain_1203_r | 7.900397573 |
| HSbrain_598_r | 7.899656198 |
| HS_from_MM_92_l | 7.887171356 |
| Hsd_from_Mmd_117_r | 7.718924828 |
| Hsd_54_l | 7.702137509 |
| HS_271_l | 7.649964483 |
| Hsd_from_Mmd_506_l | 7.638500912 |
| HSbrain_100_r | 7.597636684 |
| HSbrain_615_r | 7.513083761 |
| HS_from_PT_7_r | 7.471471439 |
| HSbrain_22_l | 7.394968873 |
| HSbrain_296_r | 7.377471663 |
| HS_132_l | 7.348116533 |
| HS_253_r | 7.309083262 |
| HS_269_l | 7.231720764 |
| HS_261_l | 7.115946746 |
| HS_from_MQbrain_1414_r | 7.115880718 |
| Hsd_from_Mmd_437_r | 7.112646234 |
| HS_from_MQbrain_603_r | 7.108558771 |
| HS_293_l | 7.088759746 |
| HS_from_PT_193_l | 7.000684 |
| HSbrain_345_l | 6.991513121 |
| HS_from_MQbrain_1636_l | 6.953922141 |
| HS_from_MQbrain_365_l | 6.941331531 |
| HS_from_MQbrain_367_r | 6.938384447 |
| Hsd_from_Mmd_318_r | 6.911841179 |
| HS_from_PT_35_r | 6.863541091 |
| HSbrain_519_l | 6.827428395 |
| HSbrain_83_r | 6.664458888 |
| HSbrain_99_r | 6.64726838 |
| HSbrain_397_l | 6.63113271 |
| HS_from_MQbrain_1110_r | 6.60657006 |
| HS_from_MQbrain_1428_r | 6.548707334 |
| HS_from_MQbrain_855_r | 6.514975985 |
| HS_from_MQbrain_1127_r | 6.478685264 |
| Hsd_from_Mmd_300_l | 6.467880633 |
| HS_from_MQbrain_1444_l | 6.459126984 |
| HS_from_MM_186_r | 6.438164347 |
| HSbrain_726_l | 6.434813111 |
| Hsd_from_Mmd_378_l | 6.433733047 |
| HS_from_MQbrain_1196_r | 6.432257948 |
| HS_from_MQbrain_1792_r | 6.412117504 |
| Hsd_from_Mmd_314_r | 6.401270974 |
| HS_from_MQbrain_161_r | 6.387946973 |
| HS_from_MQbrain_176_r | 6.3337272 |
| HS_from_MQbrain_728_r | 6.311020118 |
| HS_from_MQbrain_584_r | 6.293457493 |
| HS_250_l | 6.287211447 |
| HS_from_MQbrain_1726_l | 6.286472819 |
| HS_from_MQbrain_1836_l | 6.267799948 |
| HSbrain_770_l | 6.241026808 |
| Hsd_from_Mmd_75_r | 6.235422423 |
| HS_from_PT_99_l | 6.209015876 |
| HS_80_l | 6.188564855 |
| Hsd_from_Mmd_37_l | 6.088255723 |
| HS_from_MM_331_l | 6.063144444 |
| HS_from_PT_63_l | 6.041521667 |
| HS_from_MM_129_l | 5.994127046 |
| HSbrain_312_r | 5.989098244 |
| HS_from_MQbrain_1287_l | 5.969747859 |
| HS_from_PT_287_l | 5.921804778 |
| HS_from_MQbrain_1442_l | 5.874225415 |
| HS_from_MQbrain_1626_r | 5.841073155 |
| Hsd_from_Mmd_431_r | 5.823283134 |
| HSbrain_587_l | 5.720693567 |
| Hsd_from_Mmd_286_r | 5.6723549 |
| HS_from_MM_241_r | 5.640741712 |
| HSbrain_884_r | 5.628250928 |
| HS_from_MQbrain_1362_r | 5.601980717 |
| HS_from_MM_342_r | 5.590510164 |
| HS_from_MQbrain_681_r | 5.585694118 |
| HSbrain_149_r | 5.583342412 |
| HS_from_MQbrain_1325_r | 5.47614768 |
| HS_from_MM_20_l | 5.47139804 |
| HS_from_MQbrain_843_l | 5.455426554 |
| HSbrain_625_l | 5.435431773 |
| HSbrain_322_r | 5.365626693 |
| HS_from_MQbrain_1330_r | 5.28181558 |
| HS_from_PT_332_l | 5.2733498 |
| Hsd_from_Mmd_260_l | 5.210287976 |
| Hsd_from_Mmd_105_l | 5.193639257 |
| HSbrain_438_l | 5.166472999 |
| Hsd_96_r | 5.165955532 |
| Hsd_from_Mmd_54_l | 5.159018659 |
| HS_from_MQbrain_637_l | 5.142521242 |
| HSbrain_777_r | 5.13504626 |
| HS_from_MM_320_l | 5.118764197 |
| HSbrain_646_l | 5.06314414 |
| HS_from_MM_108_l | 5.025117442 |
| HSbrain_17_l | 4.967438933 |
| HS_from_MM_173_r | 4.943371194 |
| HS_from_MM_99_l | 4.941226582 |
| HS_from_MM_54_l | 4.939670275 |
| HS_from_MM_180_l | 4.925462788 |
| HS_301_l | 4.918434367 |
| HSbrain_215_r | 4.813707447 |
| HS_from_MQbrain_144_l | 4.773009427 |
| Hsd_from_Mmd_363_l | 4.747442615 |
| HS_from_MQbrain_1806_l | 4.715008642 |
| HS_from_PT_224_r | 4.699668621 |
| Hsd_from_Mmd_516_l | 4.635744497 |
| HSbrain_384_l | 4.544181943 |
| HSbrain_437_l | 4.53872403 |
| HS_from_MQbrain_1020_r | 4.533028897 |
| HSbrain_416_l | 4.50890556 |
| HS_from_MQbrain_653_l | 4.501537159 |
| Hsd_from_Mmd_48_r | 4.488918042 |
| Hsd_from_Mmd_163_r | 4.482580882 |
| HSbrain_298_l | 4.466165605 |
| HS_from_MQbrain_779_r | 4.449613618 |
| Hsd_from_Mmd_264_l | 4.418448309 |
| HS_from_MQbrain_1518_r | 4.395289501 |
| HS_from_MQbrain_1849_l | 4.347274863 |
| HSbrain_4_r | 4.32906393 |
| HS_from_PT_188_r | 4.316585673 |
| Hsd_from_Mmd_253_r | 4.315801417 |
| HS_from_MQbrain_366_r | 4.292586708 |
| HS_from_MM_50_r | 4.289479841 |
| HSbrain_579_l | 4.276586895 |
| HS_from_MM_270_r | 4.214386428 |
| HS_from_MM_263_r | 4.175244885 |
| HS_from_MQbrain_1946_l | 4.165279786 |
| HSbrain_1404_l | 4.149119572 |
| HS_from_PT_73_r | 4.147610682 |
| Hsd_from_Mmd_372_r | 4.138862422 |
| Hsd_45_r | 4.133836037 |
| HS_from_MQbrain_1550_r | 4.129298294 |
| HS_from_MQbrain_1840_l | 4.101477435 |
| HS_302_l | 4.060716908 |
| HS_210_r | 4.043610319 |
| HSbrain_695_r | 4.015688972 |
| Hsd_from_Mmd_319_r | 3.975166738 |
| HSbrain_538_r | 3.973394819 |
| HSbrain_775_r | 3.957435197 |
| HS_from_PT_172_r | 3.955677596 |
| HSbrain_714_r | 3.916807584 |
| Hsd_94_r | 3.914560357 |
| HSbrain_34_l | 3.900138034 |
| HS_from_MQbrain_1817_l | 3.888478757 |
| HS_from_MQbrain_133_l | 3.872882828 |
| Hsd_from_Mmd_316_l | 3.862039898 |
| HS_from_MM_353_l | 3.859645753 |
| HSbrain_199_r | 3.848060046 |
| HS_from_PT_177_l | 3.844770953 |
| HS_from_MM_131_l | 3.843700557 |
| HS_from_MM_283_l | 3.835559931 |
| HSbrain_327_r | 3.828446699 |
| HS_from_MQbrain_237_l | 3.827625487 |
| HS_from_MQbrain_1489_l | 3.827197683 |
| HS_from_PT_242_r | 3.826482783 |
| HS_from_MQbrain_675_l | 3.821332516 |
| HS_127_l | 3.813883173 |
| HSbrain_26_r | 3.807676617 |
| HS_from_MM_47_r | 3.767205302 |
| Hsd_from_Mmd_115_l | 3.754433136 |
| HSbrain_489_r | 3.754378803 |
| HSbrain_566_r | 3.737039243 |
| HSbrain_623_l | 3.719892174 |
| Hsd_101_l | 3.714373078 |
| HSbrain_444_l | 3.697175272 |
| HS_from_MQbrain_768_l | 3.670091279 |
| HSbrain_552_l | 3.649272388 |
| HS_from_MQbrain_236_l | 3.625507462 |
| HSbrain_779_l | 3.584724644 |
| Hsd_from_Mmd_382_l | 3.519705525 |
| HS_from_PT_206_l | 3.499387091 |
| HSbrain_395_r | 3.49723377 |
| HSbrain_28_r | 3.479753348 |
| HS_from_MQbrain_1770_l | 3.46187934 |
| HS_from_MM_41_l | 3.445933309 |
| Hsd_from_Mmd_67_l | 3.440647552 |
| Hsd_81_r | 3.432967686 |
| HSbrain_234_r | 3.407293997 |
| HSbrain_583_l | 3.381538102 |
| HS_175_r | 3.357952232 |
| HSbrain_1405_l | 3.356703194 |
| HSbrain_36_l | 3.355223023 |
| HSbrain_799_r | 3.32764451 |
| HSbrain_156_r | 3.316412833 |
| HS_292_r | 3.304463889 |
| HSbrain_270_l | 3.298294118 |
| HSbrain_120_l | 3.289133711 |
| HS_from_MQbrain_393_r | 3.278433239 |
| HSbrain_413_l | 3.253659453 |
| HS_from_MQbrain_571_l | 3.230584236 |
| HS_from_PT_36_l | 3.220412941 |
| HS_37_l | 3.206393454 |
| HS_52_r | 3.202332911 |
| Hsd_from_Mmd_137_l | 3.199011474 |
| HS_30_l | 3.198916759 |
| HS_from_MQbrain_241_r | 3.185534964 |
| HS_from_MQbrain_961_l | 3.171813525 |
| Hsd_from_Mmd_126_r | 3.136748756 |
| Hsd_from_Mmd_294_l | 3.120693079 |
| HS_from_MQbrain_1381_l | 3.05930194 |
| HS_from_MQbrain_309_r | 3.055368101 |
| HS_from_MQbrain_1453_l | 2.994679273 |
| HS_from_PT_271_l | 2.99221888 |
| HSbrain_198_r | 2.990967111 |
| Hsd_from_Mmd_293_l | 2.967120325 |
| HS_from_MQbrain_1598_r | 2.953508283 |
| HS_from_MM_48_r | 2.942281873 |
| HS_from_MM_230_l | 2.933799334 |
| Hsd_from_Mmd_321_r | 2.930696257 |
| HS_120_l | 2.924334019 |
| Hsd_75_r | 2.904138309 |
| HSbrain_179_r | 2.892784879 |
| HSbrain_766_l | 2.881094354 |
| Hsd_from_Mmd_376_l | 2.856829716 |
| Hsd_from_Mmd_154_l | 2.836322667 |
| HS_from_MQbrain_77_l | 2.793426308 |
| HS_from_MM_105_l | 2.764361009 |
| HS_from_MQbrain_600_r | 2.759134838 |
| HS_from_PT_288_l | 2.734365188 |
| HS_from_MQbrain_689_l | 2.726769607 |
| HS_241_r | 2.716303039 |
| HS_from_PT_84_l | 2.714709014 |
| HSbrain_86_r | 2.701204062 |
| HSbrain_245_r | 2.699435997 |
| Hsd_from_Mmd_373_r | 2.690929354 |
| HSbrain_377_r | 2.687263122 |
| HSbrain_20_r | 2.643971939 |
| HSbrain_755_l | 2.63850582 |
| HS_from_MQbrain_1146_r | 2.637847458 |
| HS_from_MQbrain_314_l | 2.61540593 |
| Hsd_57_r | 2.605334909 |
| HS_from_MQbrain_1343_r | 2.598031533 |
| HSbrain_87_r | 2.5925368 |
| Hsd_from_Mmd_346_l | 2.586537564 |
| Hsd_from_Mmd_396_r | 2.565770876 |
| Hsd_10_r | 2.526022642 |
| HS_from_MM_354_l | 2.51152459 |
| Hsd_from_Mmd_35_l | 2.507108516 |
| HS_14_l | 2.504007921 |
| Hsd_from_Mmd_503_r | 2.500865194 |
| HS_from_MQbrain_1042_l | 2.492234297 |
| HS_from_MQbrain_1137_l | 2.48333526 |
| HS_from_MQbrain_967_r | 2.462255581 |
| HSbrain_453_l | 2.447562054 |
| HS_from_MQbrain_1004_r | 2.444952219 |
| HSbrain_970_r | 2.440271917 |
| Hsd_from_Mmd_361_l | 2.438145559 |
| HS_from_MQbrain_1114_l | 2.428090643 |
| HS_from_MQbrain_116_r | 2.413035295 |
| HSbrain_650_r | 2.37442311 |
| HS_from_PT_201_r | 2.367251208 |
| HS_from_MM_84_l | 2.359765653 |
| HS_from_MM_35_l | 2.357250026 |
| HS_from_MM_218_l | 2.349494942 |
| HS_from_MQbrain_822_l | 2.343923008 |
| Hsd_from_Mmd_184_r | 2.326116517 |
| HS_205_r | 2.316073448 |
| HSbrain_682_l | 2.31200622 |
| HS_from_PT_118_r | 2.299993921 |
| HS_66_l | 2.296381916 |
| HS_from_MQbrain_1294_l | 2.290891026 |
| HS_from_MQbrain_384_l | 2.290767433 |
| HS_73_l | 2.284341705 |
| HSbrain_567_r | 2.278153257 |
| HS_23_r | 2.270142822 |
| Hsd_48_r | 2.234838488 |
| Hsd_41_r | 2.227383669 |
| HSbrain_644_r | 2.20892511 |
| HSbrain_308_l | 2.202113484 |
| HS_from_MM_288_r | 2.194320275 |
| HS_209_r | 2.177385602 |
| HSbrain_477_l | 2.16939955 |
| HS_from_MQbrain_1570_r | 2.155106437 |
| Hsd_from_Mmd_162_r | 2.145184152 |
| HS_from_MQbrain_748_l | 2.128371426 |
| HS_from_MM_127_r | 2.123969014 |
| Hsd_from_Mmd_302_l | 2.116616569 |
| HS_from_MQbrain_876_l | 2.116156997 |
| Hsd_from_Mmd_276_l | 2.097724083 |
| HSbrain_21_r | 2.096124188 |
| HS_from_MM_323_l | 2.074777286 |
| Hsd_from_Mmd_82_l | 2.072192829 |
| HSbrain_725_r | 2.042955708 |
| HS_237_l | 2.042459608 |
| HS_from_MM_68_l | 2.035960337 |
| HSbrain_264_r | 2.028164366 |

¹Cut off signal/background >2

TABLE 9a
Small RNAs recovered from faeces and differentially expressed in
adenoma vs control tissue: hairpin with highest expression

| | | adenoma vs control |
|---|---|---|
| Systematic name | signal/background¹ | (adj p)² |
| HS_217_l | 228.449758 | 0.000489293 |
| HS_169_l | 153.6041594 | 0.000489293 |
| HS_141_l | 141.8565157 | 0.000489293 |
| HS_218_r | 235.8613357 | 0.000704567 |
| HS_255_l | 161.1405598 | 0.001129352 |
| HS_232_l | 120.592551 | 0.001129352 |
| HS_262_l | 14.64955868 | 0.001446314 |
| HS_from_MM_118_r | 70.86890311 | 0.003939952 |
| HS_from_MM_309_r | 208.0343975 | 0.004006907 |
| HS_from_MM_178_r | 86.39079121 | 0.004006907 |
| HS_from_MM_169_r | 8.697594288 | 0.004006907 |
| HS_from_MM_319_l | 202.6221234 | 0.004431894 |
| HS_from_MM_334_l | 165.3471372 | 0.004431894 |
| HS_from_MM_339_r | 327.2871875 | 0.004575487 |
| HS_from_MQbrain_100_l | 133.6513061 | 0.004575487 |
| HS_from_MM_63_l | 31.12592861 | 0.004575487 |
| HS_from_MM_39_l | 15.46864042 | 0.004575487 |
| HS_from_MQbrain_1043_r | 281.5762963 | 0.004799641 |
| HS_from_MQbrain_1056_l | 71.54390765 | 0.004799641 |
| HS_from_MQbrain_1183_l | 198.4935006 | 0.004957702 |
| HS_from_MQbrain_1100_l | 165.2906308 | 0.004957702 |
| HS_from_MQbrain_1128_l | 158.837926 | 0.004957702 |
| HS_from_MQbrain_1297_l | 270.3967365 | 0.006601644 |
| HS_from_MQbrain_1292_r | 121.9448163 | 0.006601644 |
| HS_from_MQbrain_1393_r | 223.7656716 | 0.00676609 |
| HS_from_MQbrain_131_r | 202.0595323 | 0.00676609 |
| HS_from_MQbrain_1444_l | 6.459126984 | 0.007781761 |
| HS_from_MQbrain_1456_l | 199.1014452 | 0.008573112 |
| HS_from_MQbrain_1630_l | 245.6614642 | 0.008946182 |
| HS_from_MQbrain_1625_r | 238.0330045 | 0.008946182 |
| HS_from_MQbrain_1624_l | 192.3884805 | 0.008946182 |
| HS_from_MQbrain_1522_r | 112.3011721 | 0.008946182 |
| HS_from_MQbrain_1485_r | 90.24185865 | 0.008946182 |
| HS_from_MQbrain_1657_r | 116.3148049 | 0.009734613 |
| HS_from_MQbrain_1808_r | 253.1879056 | 0.01044887 |
| HS_from_MQbrain_191_l | 203.1756555 | 0.01044887 |
| HS_from_MQbrain_1884_r | 159.9476968 | 0.01044887 |
| HS_from_MQbrain_1823_r | 44.43291919 | 0.01044887 |
| HS_from_MQbrain_1792_r | 6.412117504 | 0.01044887 |
| HS_from_MQbrain_671_r | 263.6821505 | 0.010727702 |
| HS_from_MQbrain_71_r | 252.4897985 | 0.010727702 |
| HS_from_MQbrain_330_r | 236.6286556 | 0.010727702 |
| HS_from_MQbrain_20_r | 229.8506554 | 0.010727702 |
| HS_from_MQbrain_629_l | 195.1102679 | 0.010727702 |
| HS_from_MQbrain_350_l | 189.461419 | 0.010727702 |
| HS_from_MQbrain_2380_r | 187.2947993 | 0.010727702 |
| HS_from_MQbrain_207_l | 157.9140691 | 0.010727702 |
| HS_from_MQbrain_349_l | 135.3020631 | 0.010727702 |
| HS_from_MQbrain_619_r | 122.6749499 | 0.010727702 |
| HS_from_MQbrain_194_l | 110.0069304 | 0.010727702 |
| HS_from_MQbrain_212_r | 83.02395006 | 0.010727702 |
| HS_from_MQbrain_549_l | 34.06783333 | 0.010727702 |
| HS_from_MQbrain_441_l | 32.28225695 | 0.010727702 |
| HS_from_MQbrain_55_l | 15.80840103 | 0.010727702 |
| HS_from_MQbrain_839_r | 92.94488973 | 0.011699489 |
| HS_from_MQbrain_79_r | 48.19976833 | 0.011699489 |
| HS_from_MQbrain_917_r | 280.5140354 | 0.012506309 |
| HS_from_MQbrain_940_r | 239.4870149 | 0.012506309 |
| HS_from_MQbrain_965_l | 235.4627068 | 0.012506309 |
| HS_from_PT_237_r | 288.8596386 | 0.013034759 |
| HS_from_PT_190_r | 138.4453126 | 0.013034759 |
| HS_from_PT_257_r | 27.728139 | 0.013034759 |
| HS_from_PT_40_r | 19.96645961 | 0.013034759 |
| HS_from_PT_36_l | 3.220412941 | 0.013034759 |
| HS_from_PT_201_r | 2.367251208 | 0.013034759 |
| HSbrain_171_l | 255.6856718 | 0.017198956 |
| HSbrain_148_r | 224.0526161 | 0.017198956 |
| HSbrain_102_r | 200.4155278 | 0.017198956 |
| HSbrain_158_l | 169.343928 | 0.017198956 |
| HSbrain_1305_l | 145.0316211 | 0.017198956 |
| HSbrain_1_l | 76.8373873 | 0.017198956 |
| HSbrain_106_r | 74.6228739 | 0.017198956 |
| HSbrain_12_l | 53.76557022 | 0.017198956 |
| HSbrain_231_l | 226.0585846 | 0.018600058 |
| HSbrain_321_r | 211.435012 | 0.018600058 |
| HSbrain_243_l | 182.2549801 | 0.018600058 |
| HSbrain_224_r | 81.77304743 | 0.018600058 |
| HSbrain_2_l | 49.67189807 | 0.018600058 |
| HSbrain_426_r | 262.208223 | 0.019454312 |
| HSbrain_420_r | 228.3614012 | 0.019454312 |
| HSbrain_359_r | 226.1695269 | 0.019454312 |
| HSbrain_337_l | 85.40127884 | 0.019454312 |
| HSbrain_446_l | 14.33402718 | 0.019454312 |
| HSbrain_377_r | 2.687263122 | 0.019454312 |
| HSbrain_485_r | 258.4877461 | 0.021088597 |
| HSbrain_527_r | 228.8704808 | 0.021088597 |
| HSbrain_483_l | 220.2067855 | 0.021088597 |
| HSbrain_503_r | 181.710235 | 0.021088597 |
| HSbrain_540_r | 19.28862768 | 0.021088597 |
| HSbrain_583_l | 3.381538102 | 0.021088597 |
| HSbrain_709_l | 274.820351 | 0.022745616 |
| HSbrain_629_l | 254.3371274 | 0.022745616 |
| HSbrain_6_r | 244.8860331 | 0.022745616 |
| HSbrain_597_r | 208.6310714 | 0.022745616 |
| HSbrain_636_l | 159.8695431 | 0.022745616 |
| HSbrain_617_l | 59.52352443 | 0.022745616 |
| Hsd_18_l | 237.5322474 | 0.023736962 |
| HSbrain_723_l | 236.0439796 | 0.023736962 |
| HSbrain_8_r | 227.4862458 | 0.023736962 |
| HSbrain_75_l | 227.0689221 | 0.023736962 |
| HSbrain_916_l | 211.4233793 | 0.023736962 |
| HSbrain_801_r | 195.7930985 | 0.023736962 |
| HSbrain_722_l | 180.1260425 | 0.023736962 |
| HSbrain_727_l | 136.0474595 | 0.023736962 |
| HSbrain_811_r | 131.6313177 | 0.023736962 |
| HSbrain_753_r | 41.77257724 | 0.023736962 |
| Hsd_70_l | 292.1907862 | 0.024762366 |
| Hsd_from_Mmd_92_r | 212.1087908 | 0.024762366 |
| Hsd_49_r | 162.0054723 | 0.024762366 |
| Hsd_from_Mmd_527_r | 126.3935973 | 0.024762366 |
| Hsd_from_Mmd_485_r | 95.1476108 | 0.024762366 |
| Hsd_from_Mmd_298_r | 48.27315482 | 0.024762366 |
| Hsd_from_Mmd_533_l | 37.74138535 | 0.024762366 |
| Hsd_from_Mmd_452_l | 21.7681635 | 0.024762366 |
| Hsd_from_Mmd_473_l | 15.1413037 | 0.024762366 |
| Hsd_from_Mmd_117_r | 7.718924828 | 0.024762366 |
| HS_from_MQbrain_734_l | 272.5872546 | 0.026800941 |
| HS_from_MQbrain_1684_l | 158.2373516 | 0.026800941 |
| HS_from_MQbrain_509_r | 100.1137811 | 0.026800941 |
| HS_from_MM_131_l | 3.843700557 | 0.026800941 |
| HS_from_MQbrain_74_r | 225.8922409 | 0.028655448 |
| HSbrain_318_l | 221.848446 | 0.028655448 |
| HS_from_MQbrain_391_r | 219.2029092 | 0.028655448 |
| HS_from_PT_139_l | 85.75830736 | 0.028655448 |
| HSbrain_259_r | 334.9867442 | 0.02997784 |
| HS_from_MQbrain_1832_l | 275.5897539 | 0.02997784 |
| HSbrain_713_l | 225.8656496 | 0.02997784 |
| HS_106_l | 197.2533991 | 0.02997784 |
| HS_from_MQbrain_918_l | 186.6733108 | 0.02997784 |
| HS_from_PT_299_l | 158.8812184 | 0.02997784 |
| HS_from_MQbrain_1169_l | 105.7095098 | 0.02997784 |
| HSbrain_546_r | 19.96726522 | 0.02997784 |
| HS_from_MQbrain_1392_r | 288.9210201 | 0.03224042 |
| HS_118_l | 214.1827525 | 0.03224042 |
| HS_from_MQbrain_1863_l | 197.0519986 | 0.03224042 |
| HS_from_MQbrain_1443_r | 179.1434855 | 0.03224042 |
| HS_from_MQbrain_1445_l | 154.9673281 | 0.03224042 |
| HS_from_PT_103_l | 74.08268376 | 0.03224042 |
| HS_from_MQbrain_1469_r | 28.13492378 | 0.03224042 |
| HSbrain_316_l | 339.4367001 | 0.03374871 |
| HSbrain_41_l | 239.6624516 | 0.03374871 |
| HSbrain_165_l | 181.1832002 | 0.03374871 |
| HSbrain_760_l | 136.864711 | 0.03374871 |
| Hsd_from_Mmd_81_l | 114.8866476 | 0.03374871 |
| HS_231_r | 112.1781103 | 0.03374871 |
| HSbrain_458_l | 77.3089971 | 0.03374871 |
| HS_from_MQbrain_800_r | 57.33831283 | 0.03374871 |
| HS_from_MQbrain_435_l | 50.08067692 | 0.03374871 |
| HS_from_MQbrain_1690_l | 21.66605198 | 0.03374871 |
| Hsd_102_l | 175.5645249 | 0.036099692 |
| HS_from_MQbrain_1340_l | 157.6981977 | 0.036099692 |
| HS_from_MQbrain_1513_r | 128.9407151 | 0.036099692 |
| HS_from_MQbrain_498_r | 342.07876 | 0.038273758 |
| HS_from_MQbrain_1315_l | 181.848048 | 0.038273758 |
| HS_from_MQbrain_282_l | 163.9894054 | 0.038273758 |
| HSbrain_261_l | 83.18937251 | 0.038273758 |
| HSbrain_700_r | 59.35494614 | 0.038273758 |
| HSbrain_543_r | 26.09736904 | 0.038273758 |
| HSbrain_92_l | 290.3024103 | 0.040151024 |
| HSbrain_109_l | 284.6354613 | 0.040151024 |
| HS_from_PT_43_l | 279.7988575 | 0.040151024 |
| HS_from_MQbrain_1747_r | 278.4365165 | 0.040151024 |
| HS_56_r | 243.5447343 | 0.040151024 |
| HS_from_MQbrain_951_l | 230.432556 | 0.040151024 |
| HS_from_MQbrain_470_l | 131.1632076 | 0.040151024 |
| HS_189_l | 117.9592496 | 0.040151024 |
| Hsd_from_Mmd_436_r | 90.76582879 | 0.040151024 |
| HS_from_MQbrain_1080_l | 70.18685041 | 0.040151024 |
| HS_from_MM_276_l | 21.75172107 | 0.040151024 |
| HS_from_MQbrain_782_r | 261.0759728 | 0.042549287 |
| Hsd_42_r | 260.2142458 | 0.042549287 |
| HS_from_MQbrain_1227_l | 243.816199 | 0.042549287 |
| HS_from_MQbrain_1388_l | 214.966548 | 0.042549287 |
| HS_from_MQbrain_1844_r | 187.6108805 | 0.042549287 |
| Hsd_from_Mmd_261_l | 184.5887939 | 0.042549287 |
| HS_15_l | 177.8515513 | 0.042549287 |
| HSbrain_1410_l | 66.2043953 | 0.042549287 |
| HSbrain_284_l | 257.0803228 | 0.045725655 |
| HS_from_PT_70_r | 202.4337957 | 0.045725655 |
| Hsd_63_r | 191.7137571 | 0.045725655 |
| HS_from_MQbrain_1143_r | 136.8939224 | 0.045725655 |
| Hsd_from_Mmd_34_r | 114.2335916 | 0.045725655 |
| HS_from_MQbrain_1075_l | 258.9475578 | 0.048378078 |
| HSbrain_703_l | 247.3420423 | 0.048378078 |
| HS_from_MQbrain_539_r | 234.1938897 | 0.048378078 |
| HS_from_MQbrain_472_r | 225.3108408 | 0.048378078 |
| HSbrain_1362_l | 215.6819962 | 0.048378078 |
| HS_114_l | 214.0153294 | 0.048378078 |
| HS_from_MQbrain_1773_l | 173.1308074 | 0.048378078 |
| HS_from_MQbrain_92_l | 153.9678306 | 0.048378078 |
| HSbrain_25_r | 68.45071483 | 0.048378078 |
| HS_from_MM_1_r | 42.18437325 | 0.048378078 |
| HS_from_MQbrain_1686_l | 381.0649897 | 0.050130838 |
| HSbrain_652_l | 240.7492888 | 0.050130838 |
| HS_from_MQbrain_343_l | 214.8585663 | 0.050130838 |
| HS_from_MQbrain_901_l | 214.2516994 | 0.050130838 |
| HSbrain_84_l | 200.5841432 | 0.050130838 |
| HS_from_MQbrain_1283_l | 200.1271847 | 0.050130838 |
| HS_215_l | 186.3533099 | 0.050130838 |
| HS_from_MQbrain_774_r | 164.6264718 | 0.050130838 |
| HS_from_MQbrain_1610_r | 128.7979365 | 0.050130838 |
| HSbrain_585_l | 124.1437027 | 0.050130838 |
| HS_from_PT_136_l | 92.74159284 | 0.050130838 |
| HS_from_MQbrain_315_l | 85.77155971 | 0.050130838 |
| HS_93_r | 17.4404153 | 0.050130838 |
| HS_from_MQbrain_1239_l | 225.7278011 | 0.053159859 |
| Hsd_37_l | 213.5626033 | 0.053159859 |
| HS_131_r | 209.6414321 | 0.053159859 |
| HS_from_MQbrain_873_r | 206.5787776 | 0.053159859 |
| HS_from_MQbrain_1395_r | 196.1672376 | 0.053159859 |
| HS_from_MQbrain_1484_l | 180.1270647 | 0.053159859 |
| Hsd_76_r | 80.46787511 | 0.053159859 |
| Hsd_6_l | 53.15042029 | 0.053159859 |
| HSbrain_442_r | 18.08359556 | 0.053159859 |
| HS_from_MQbrain_1620_l | 307.2296305 | 0.05566524 |
| HSbrain_85_l | 264.5381922 | 0.05566524 |
| HS_from_MM_115_l | 244.343368 | 0.05566524 |
| HS_from_MQbrain_56_l | 221.5946492 | 0.05566524 |
| HSbrain_419_l | 219.8793357 | 0.05566524 |
| HS_from_MQbrain_664_l | 217.4766553 | 0.05566524 |
| HS_from_MQbrain_1674_l | 188.625056 | 0.05566524 |
| HS_from_MM_49_l | 186.4616319 | 0.05566524 |
| HSbrain_239_l | 98.26806599 | 0.05566524 |
| HS_62_l | 66.38174565 | 0.05566524 |
| HS_59_l | 66.17528144 | 0.05566524 |
| HSbrain_704_l | 45.12744924 | 0.05566524 |
| HSbrain_671_r | 22.1043858 | 0.05566524 |
| HS_from_PT_321_r | 9.355284488 | 0.05566524 |
| HSbrain_719_l | 9.057424006 | 0.05566524 |
| HS_from_MQbrain_1119_l | 316.6560497 | 0.058253679 |
| HSbrain_35_r | 279.8399868 | 0.058253679 |
| HS_from_MQbrain_974_l | 247.9593335 | 0.058253679 |
| HS_190_l | 245.2188679 | 0.058253679 |
| HS_from_MQbrain_810_l | 225.1889153 | 0.058253679 |
| HS_from_PT_121_r | 221.5383439 | 0.058253679 |
| HSbrain_123_l | 203.4486322 | 0.058253679 |
| HSbrain_300_l | 182.522588 | 0.058253679 |
| HS_from_MQbrain_883_r | 179.9509263 | 0.058253679 |
| HS_from_MQbrain_1374_l | 176.8739274 | 0.058253679 |
| HS_from_MQbrain_1300_l | 153.3945718 | 0.058253679 |
| Hsd_7_r | 23.39907905 | 0.058253679 |
| HS_from_MQbrain_70_l | 285.3653998 | 0.061046963 |
| HS_304_r | 244.034349 | 0.061046963 |
| HS_150_l | 201.5054656 | 0.061046963 |
| HS_from_MQbrain_128_r | 194.6783323 | 0.061046963 |
| HSbrain_409_r | 170.7071739 | 0.061046963 |
| HS_36_l | 91.72099648 | 0.061046963 |
| HS_from_MQbrain_991_r | 72.5119085 | 0.061046963 |
| HS_from_MQbrain_1354_l | 72.49188083 | 0.061046963 |
| HS_from_MQbrain_1404_r | 27.3954386 | 0.061046963 |
| HSbrain_214_l | 19.36197308 | 0.061046963 |
| HS_from_MQbrain_750_r | 237.0499841 | 0.064770288 |
| HS_180_r | 200.4582411 | 0.064770288 |
| HSbrain_116_l | 198.8454747 | 0.064770288 |
| HSbrain_506_l | 81.68257158 | 0.064770288 |
| Hsd_81_r | 3.432967686 | 0.064770288 |
| HSbrain_755_l | 2.63850582 | 0.064770288 |
| HS_from_MQbrain_745_r | 271.8779897 | 0.067503134 |
| HS_from_PT_112_l | 227.4432793 | 0.067503134 |
| HSbrain_212_l | 224.1025327 | 0.067503134 |
| HS_from_PT_339_l | 214.3823639 | 0.067503134 |
| Hsd_62_l | 208.0883252 | 0.067503134 |
| HS_from_MQbrain_1427_l | 181.7148181 | 0.067503134 |
| HSbrain_330_l | 174.1826185 | 0.067503134 |
| HS_from_MQbrain_239_r | 173.8939829 | 0.067503134 |
| HSbrain_139_l | 172.7598914 | 0.067503134 |
| HS_203_r | 137.1520157 | 0.067503134 |
| HSbrain_572_r | 64.68912173 | 0.067503134 |
| Hsd_from_Mmd_311_l | 32.03234884 | 0.067503134 |
| Hsd_from_Mmd_524_r | 14.7018346 | 0.067503134 |
| HS_from_MM_342_r | 5.590510164 | 0.067503134 |
| HS_from_MQbrain_1376_l | 319.0687179 | 0.069749265 |
| Hsd_50_l | 297.3692697 | 0.069749265 |
| HS_from_MQbrain_1771_r | 247.8706875 | 0.069749265 |
| HS_from_MQbrain_723_r | 237.156234 | 0.069749265 |
| HSbrain_610_r | 220.5552561 | 0.069749265 |
| HS_from_MQbrain_1841_r | 214.8445757 | 0.069749265 |
| Hsd_from_Mmd_330_l | 200.078931 | 0.069749265 |
| HS_from_MQbrain_251_r | 172.0989873 | 0.069749265 |
| HS_from_PT_187_l | 128.3298072 | 0.069749265 |
| HS_from_MQbrain_997_l | 90.55621189 | 0.069749265 |
| HS_from_MQbrain_641_r | 72.1752804 | 0.069749265 |
| HSbrain_777_r | 5.13504626 | 0.069749265 |
| HS_from_MQbrain_366_r | 4.292586708 | 0.069749265 |
| HSbrain_245_r | 2.699435997 | 0.069749265 |
| HS_from_MQbrain_1219_l | 301.2674923 | 0.072349461 |
| HS_from_MM_289_r | 285.4582707 | 0.072349461 |
| HS_from_MQbrain_1082_l | 265.0804535 | 0.072349461 |
| HS_from_MM_295_r | 264.8300478 | 0.072349461 |
| HSbrain_1364_l | 251.9113433 | 0.072349461 |
| HSbrain_749_r | 231.9379385 | 0.072349461 |
| HSbrain_301_l | 226.109583 | 0.072349461 |
| HS_from_MQbrain_1561_l | 207.9055923 | 0.072349461 |
| HS_from_MQbrain_1280_l | 188.9766392 | 0.072349461 |
| HS_from_MQbrain_433_l | 186.8433165 | 0.072349461 |
| HS_from_PT_170_l | 185.2808375 | 0.072349461 |
| HS_from_MQbrain_1565_r | 183.33017 | 0.072349461 |
| HSbrain_1363_l | 175.794645 | 0.072349461 |
| HS_from_MQbrain_1658_r | 135.9727881 | 0.072349461 |
| Hsd_from_Mmd_333_r | 122.8958947 | 0.072349461 |
| HSbrain_747_l | 100.2134098 | 0.072349461 |
| HS_49_r | 8.969465473 | 0.072349461 |
| HSbrain_788_l | 249.4670383 | 0.076797167 |
| HSbrain_767_r | 227.1006295 | 0.076797167 |
| HS_from_MQbrain_930_r | 167.8363796 | 0.076797167 |
| HS_from_MQbrain_687_r | 109.1779088 | 0.076797167 |
| HS_from_MQbrain_224_l | 69.6781412 | 0.076797167 |
| HS_from_MM_219_l | 47.65091531 | 0.076797167 |
| HSbrain_344_l | 44.15992199 | 0.076797167 |
| HS_from_MQbrain_1414_r | 7.115880718 | 0.076797167 |
| HS_from_MM_353_l | 3.859645753 | 0.076797167 |
| HS_from_MQbrain_1777_l | 271.5768067 | 0.079954597 |
| HS_from_MQbrain_576_l | 267.323134 | 0.079954597 |
| Hsd_from_Mmd_204_l | 251.2502954 | 0.079954597 |
| HS_from_PT_102_l | 248.6580593 | 0.079954597 |
| HSbrain_317_l | 234.263649 | 0.079954597 |
| HS_119_l | 233.7784735 | 0.079954597 |
| HS_from_MQbrain_1700_l | 193.6041577 | 0.079954597 |
| HS_from_MQbrain_1745_r | 191.5196529 | 0.079954597 |
| HSbrain_455_r | 178.2419961 | 0.079954597 |
| Hsd_11_l | 164.5093304 | 0.079954597 |
| HS_from_MQbrain_1520_r | 158.8609156 | 0.079954597 |
| HS_from_PT_148_l | 151.4484249 | 0.079954597 |
| HS_from_MQbrain_1637_r | 147.2561547 | 0.079954597 |
| Hsd_33_l | 33.05107162 | 0.079954597 |
| HSbrain_1406_l | 17.08291783 | 0.079954597 |
| Hsd_101_l | 3.714373078 | 0.079954597 |
| HS_from_MQbrain_1102_l | 303.4601583 | 0.083930001 |
| HS_from_MM_195_r | 276.4162313 | 0.083930001 |
| HS_57_l | 250.390488 | 0.083930001 |
| Hsd_from_Mmd_501_r | 243.1733615 | 0.083930001 |
| HS_from_MQbrain_715_l | 216.9853807 | 0.083930001 |
| HSbrain_56_l | 210.9521542 | 0.083930001 |
| HS_from_PT_20_r | 188.3393313 | 0.083930001 |
| HSbrain_548_l | 185.4850722 | 0.083930001 |
| HSbrain_286_l | 131.389336 | 0.083930001 |
| HS_from_MQbrain_154_l | 112.5010282 | 0.083930001 |
| HS_from_MQbrain_769_r | 87.406602 | 0.083930001 |
| HSbrain_389_l | 9.742590244 | 0.083930001 |
| HSbrain_711_r | 8.507528601 | 0.083930001 |
| HS_from_MQbrain_1325_r | 5.47614768 | 0.083930001 |
| HS_from_MM_347_l | 350.4004069 | 0.088143181 |
| HS_from_MQbrain_686_r | 259.8101317 | 0.088143181 |
| HS_from_MQbrain_806_l | 220.5421957 | 0.088143181 |
| HS_102_l | 205.6195359 | 0.088143181 |
| HSbrain_124_l | 199.2157195 | 0.088143181 |
| HS_from_MM_165_l | 194.0305909 | 0.088143181 |
| HSbrain_590_r | 190.8385095 | 0.088143181 |
| HS_from_MQbrain_291_l | 188.7078132 | 0.088143181 |
| HS_from_MQbrain_948_r | 176.1262044 | 0.088143181 |
| HS_from_MM_229_r | 173.5953652 | 0.088143181 |
| HS_from_MQbrain_1064_r | 160.6505983 | 0.088143181 |
| HSbrain_170_r | 58.09549284 | 0.088143181 |
| HSbrain_530_l | 49.2660315 | 0.088143181 |
| HS_from_PT_254_r | 38.90953686 | 0.088143181 |
| HS_80_l | 6.188564855 | 0.088143181 |
| HS_from_MQbrain_1626_r | 5.841073155 | 0.088143181 |
| HSbrain_637_l | 278.8901829 | 0.09152926 |
| Hsd_from_Mmd_120_r | 256.1765933 | 0.09152926 |
| HS_from_MQbrain_550_l | 253.7757692 | 0.09152926 |
| HS_from_MQbrain_1758_r | 252.4287529 | 0.09152926 |
| HSbrain_1360_l | 246.1741425 | 0.09152926 |
| HS_from_MQbrain_1885_r | 208.5987571 | 0.09152926 |
| HSbrain_642_r | 198.8901506 | 0.09152926 |
| HS_from_PT_95_r | 187.7990727 | 0.09152926 |
| HS_4_r | 186.5048701 | 0.09152926 |
| HS_from_MQbrain_1592_l | 169.5192576 | 0.09152926 |
| Hsd_from_Mmd_161_l | 111.7055777 | 0.09152926 |
| HS_from_MQbrain_1037_r | 101.466981 | 0.09152926 |
| HSbrain_237_r | 20.78891804 | 0.09152926 |
| HS_from_MQbrain_1494_l | 14.7687191 | 0.09152926 |
| Hsd_from_Mmd_378_l | 6.433733047 | 0.09152926 |
| HS_from_MQbrain_1726_l | 6.286472819 | 0.09152926 |
| HS_129_l | 287.8783772 | 0.095394629 |
| HSbrain_717_l | 241.5872727 | 0.095394629 |
| HSbrain_708_l | 229.246264 | 0.095394629 |
| HSbrain_691_l | 201.4670994 | 0.095394629 |
| Hsd_from_Mmd_519_r | 190.1006667 | 0.095394629 |
| Hsd_79_l | 181.0929049 | 0.095394629 |
| HS_288_l | 171.5705836 | 0.095394629 |
| HS_from_MQbrain_1228_r | 162.3489481 | 0.095394629 |
| HSbrain_557_r | 143.8626534 | 0.095394629 |
| HS_from_PT_74_r | 107.2883837 | 0.095394629 |
| HSbrain_113_r | 100.6056081 | 0.095394629 |
| HS_from_MQbrain_255_l | 64.86378183 | 0.095394629 |
| HSbrain_481_r | 29.96985078 | 0.095394629 |
| HS_from_MQbrain_1201_r | 23.9141836 | 0.095394629 |
| Hsd_from_Mmd_223_r | 19.94197059 | 0.095394629 |
| HS_from_MQbrain_1648_r | 18.04974603 | 0.095394629 |
| HS_from_MQbrain_574_l | 287.7017103 | 0.098921026 |
| HSbrain_1425_l | 283.5640729 | 0.098921026 |
| Hsd_from_Mmd_480_r | 278.0451339 | 0.098921026 |
| HS_from_MQbrain_868_l | 260.4645119 | 0.098921026 |
| HSbrain_240_l | 253.8771621 | 0.098921026 |
| HS_157_l | 250.822208 | 0.098921026 |
| HS_from_MM_103_l | 246.9217731 | 0.098921026 |
| HSbrain_769_r | 228.6374424 | 0.098921026 |
| HSbrain_305_r | 219.5650075 | 0.098921026 |
| HS_from_MQbrain_1382_l | 216.2287654 | 0.098921026 |
| Hsd_from_Mmd_176_l | 197.2483957 | 0.098921026 |
| HSbrain_415_l | 148.8998605 | 0.098921026 |
| HS_from_MQbrain_1213_l | 136.145634 | 0.098921026 |
| HS_from_MQbrain_2670_r | 88.75898562 | 0.098921026 |
| HS_from_MM_313_l | 43.35911147 | 0.098921026 |
| HS_from_MQbrain_1753_r | 15.00695265 | 0.098921026 |
| Hsd_from_Mmd_325_l | 12.27846175 | 0.098921026 |
| HS_from_MM_34_l | 7.978839103 | 0.098921026 |
| HSbrain_86_r | 2.701204062 | 0.098921026 |
| HS_from_MQbrain_1355_r | 309.6691714 | 0.103393572 |
| HS_from_MQbrain_1459_l | 257.1079597 | 0.103393572 |
| HSbrain_677_l | 223.7296488 | 0.103393572 |
| HS_from_MQbrain_609_l | 213.1909341 | 0.103393572 |
| HS_from_MQbrain_943_l | 199.1939949 | 0.103393572 |
| HSbrain_596_r | 183.4234846 | 0.103393572 |
| HS_from_PT_22_l | 169.7548575 | 0.103393572 |
| HS_from_MQbrain_1744_r | 145.1169393 | 0.103393572 |
| HSbrain_482_l | 110.0149117 | 0.103393572 |
| HSbrain_94_l | 96.33702157 | 0.103393572 |
| HSbrain_343_l | 82.34298795 | 0.103393572 |
| HS_68_l | 77.85150167 | 0.103393572 |
| HS_172_r | 66.96978425 | 0.103393572 |
| HS_from_MQbrain_906_r | 47.10729944 | 0.103393572 |
| HSbrain_504_r | 45.47207589 | 0.103393572 |
| HS_from_MQbrain_1784_r | 8.765325591 | 0.103393572 |
| HS_from_MQbrain_1442_l | 5.874225415 | 0.103393572 |
| HS_from_MQbrain_114_r | 298.9587162 | 0.108118292 |
| HS_from_MQbrain_546_l | 288.7897335 | 0.108118292 |
| HSbrain_536_r | 269.1404787 | 0.108118292 |
| Hsd_from_Mmd_291_r | 266.1652183 | 0.108118292 |
| HS_from_MQbrain_1627_l | 251.4373669 | 0.108118292 |
| HS_101_l | 239.0332153 | 0.108118292 |
| HSbrain_643_l | 212.7527559 | 0.108118292 |
| HS_from_MQbrain_1366_l | 212.34134 | 0.108118292 |
| HS_from_PT_220_l | 201.2064584 | 0.108118292 |
| HSbrain_280_l | 188.6563025 | 0.108118292 |
| HS_from_MQbrain_374_r | 170.5241907 | 0.108118292 |
| HS_from_MQbrain_80_l | 62.18301452 | 0.108118292 |
| Hsd_from_Mmd_478_l | 24.52473283 | 0.108118292 |
| Hsd_from_Mmd_249_r | 12.73986084 | 0.108118292 |
| HS_from_PT_7_r | 7.471471439 | 0.108118292 |
| HSbrain_384_l | 4.544181943 | 0.108118292 |
| HS_from_MQbrain_1840_l | 4.101477435 | 0.108118292 |
| HS_from_MQbrain_604_l | 289.730194 | 0.113099484 |
| HSbrain_328_l | 280.7719926 | 0.113099484 |
| HSbrain_520_l | 236.1868036 | 0.113099484 |
| HSbrain_227_r | 226.199823 | 0.113099484 |
| HSbrain_267_l | 214.022333 | 0.113099484 |
| HS_from_MQbrain_1478_l | 192.6931886 | 0.113099484 |
| HS_from_MQbrain_921_l | 192.1335465 | 0.113099484 |
| HSbrain_764_r | 117.9645256 | 0.113099484 |
| HS_from_MQbrain_1502_l | 54.14110189 | 0.113099484 |
| Hsd_from_Mmd_391_l | 43.63503635 | 0.113099484 |
| HSbrain_412_l | 37.17793298 | 0.113099484 |
| HSbrain_159_l | 19.09259883 | 0.113099484 |
| HS_from_MQbrain_105_l | 16.80746034 | 0.113099484 |
| HSbrain_145_l | 267.9664391 | 0.11858389 |
| HS_from_MQbrain_1889_l | 262.029435 | 0.11858389 |
| HS_from_MQbrain_999_l | 259.1426385 | 0.11858389 |
| Hsd_from_Mmd_518_r | 238.0578899 | 0.11858389 |
| HS_289_l | 236.2167396 | 0.11858389 |
| HSbrain_664_r | 222.5408696 | 0.11858389 |
| HS_from_MQbrain_1750_r | 217.7023298 | 0.11858389 |
| HS_from_MQbrain_1834_r | 187.1781356 | 0.11858389 |
| Hsd_from_Mmd_93_l | 186.4505924 | 0.11858389 |
| HS_from_MQbrain_592_l | 181.6866459 | 0.11858389 |
| HS_from_MQbrain_1590_l | 163.8114142 | 0.11858389 |
| HS_from_MQbrain_1487_r | 119.4385433 | 0.11858389 |
| HS_from_MQbrain_502_r | 118.3776111 | 0.11858389 |
| HS_98_r | 330.803132 | 0.12422104 |
| HS_25_r | 313.2035431 | 0.12422104 |
| Hsd_from_Mmd_237_l | 306.9716516 | 0.12422104 |
| Hsd_17_l | 255.7570196 | 0.12422104 |
| HS_from_MM_87_l | 240.7726111 | 0.12422104 |
| HS_from_MQbrain_789_l | 239.2174834 | 0.12422104 |
| HS_from_MQbrain_1184_r | 208.6202764 | 0.12422104 |
| HSbrain_50_l | 203.7449536 | 0.12422104 |
| HS_287_l | 203.2812692 | 0.12422104 |
| HS_from_MM_79_l | 199.5386163 | 0.12422104 |
| HSbrain_570_l | 194.0529436 | 0.12422104 |
| HS_from_MQbrain_490_r | 162.3944533 | 0.12422104 |
| HS_from_MQbrain_1304_r | 154.625417 | 0.12422104 |
| Hsd_from_Mmd_328_r | 117.324613 | 0.12422104 |
| HS_from_MQbrain_1109_l | 62.73503843 | 0.12422104 |
| HSbrain_131_l | 51.07910404 | 0.12422104 |
| HS_from_MQbrain_1384_r | 26.20937179 | 0.12422104 |
| HS_from_MQbrain_161_r | 6.387946973 | 0.12422104 |
| HSbrain_152_l | 261.0331392 | 0.129399391 |
| HS_from_MQbrain_36_r | 247.5789144 | 0.129399391 |
| HS_from_MQbrain_1574_l | 236.4214286 | 0.129399391 |
| HS_from_MQbrain_1794_r | 227.1557202 | 0.129399391 |
| HSbrain_204_r | 217.0811451 | 0.129399391 |
| HSbrain_72_l | 206.0852876 | 0.129399391 |
| HS_from_MQbrain_1776_l | 205.2456623 | 0.129399391 |
| HS_109_l | 200.1919676 | 0.129399391 |
| HS_from_MM_304_r | 195.9761008 | 0.129399391 |
| HS_from_MQbrain_1839_l | 186.2075177 | 0.129399391 |
| HSbrain_448_l | 162.0375436 | 0.129399391 |
| HS_85_l | 96.48110294 | 0.129399391 |
| HS_from_MQbrain_597_l | 8.606774472 | 0.129399391 |
| Hsd_from_Mmd_363_l | 4.747442615 | 0.129399391 |
| HSbrain_970_r | 2.440271917 | 0.129399391 |
| HS_from_MQbrain_684_r | 341.5414126 | 0.134970683 |
| Hsd_74_r | 274.0203487 | 0.134970683 |
| HS_from_MQbrain_1523_r | 272.3792039 | 0.134970683 |
| HS_from_MQbrain_540_l | 268.4654883 | 0.134970683 |
| HS_from_MQbrain_1318_l | 223.6306158 | 0.134970683 |
| HS_from_MQbrain_99_r | 220.3197222 | 0.134970683 |
| HS_from_MQbrain_234_r | 205.0056018 | 0.134970683 |
| Hsd_from_Mmd_101_r | 177.7313602 | 0.134970683 |
| HS_from_PT_250_r | 169.7827673 | 0.134970683 |
| HS_from_MQbrain_1692_l | 158.6415708 | 0.134970683 |
| HS_from_MQbrain_1097_r | 128.6204149 | 0.134970683 |
| HS_from_MM_185_r | 68.74396066 | 0.134970683 |
| HS_from_MQbrain_1081_r | 20.97667628 | 0.134970683 |
| HS_261_l | 7.115946746 | 0.134970683 |
| HS_from_MQbrain_1806_l | 4.715008642 | 0.134970683 |
| HSbrain_108_l | 266.7753944 | 0.140323812 |
| HS_219_l | 254.6760586 | 0.140323812 |
| HS_from_PT_178_l | 242.7023061 | 0.140323812 |
| HS_from_MQbrain_1855_l | 238.2044165 | 0.140323812 |
| HS_168_r | 235.6020265 | 0.140323812 |
| HSbrain_361_l | 234.5281831 | 0.140323812 |
| HS_121_r | 231.1610876 | 0.140323812 |
| Hsd_from_Mmd_123_l | 230.1089517 | 0.140323812 |
| HSbrain_144_r | 224.6332678 | 0.140323812 |
| HS_161_r | 216.0695424 | 0.140323812 |
| HS_from_MQbrain_467_l | 209.268142 | 0.140323812 |
| HS_from_MQbrain_22_r | 187.1051151 | 0.140323812 |
| HSbrain_716_l | 180.5676912 | 0.140323812 |
| HS_53_r | 140.0976088 | 0.140323812 |
| HS_41_r | 112.6985514 | 0.140323812 |
| HSbrain_737_r | 53.36757802 | 0.140323812 |
| Hsd_from_Mmd_523_l | 10.60175496 | 0.140323812 |
| HS_241_r | 2.716303039 | 0.140323812 |
| HS_from_MQbrain_1675_r | 288.3970715 | 0.146822481 |
| HS_from_MQbrain_67_l | 255.5891945 | 0.146822481 |
| HS_from_PT_219_l | 253.1829244 | 0.146822481 |
| HS_from_MQbrain_1807_l | 220.9858095 | 0.146822481 |
| HS_from_MQbrain_97_r | 220.2493082 | 0.146822481 |
| HSbrain_130_l | 216.1273018 | 0.146822481 |
| HS_from_MQbrain_1222_r | 214.0598655 | 0.146822481 |
| HS_from_MQbrain_1493_r | 207.5291507 | 0.146822481 |
| HS_from_MQbrain_1695_r | 204.2688796 | 0.146822481 |
| HS_from_MQbrain_1036_r | 203.1951911 | 0.146822481 |
| Hsd_77_r | 200.6733718 | 0.146822481 |
| HS_from_MQbrain_1030_l | 164.3970563 | 0.146822481 |
| HSbrain_663_r | 155.8547049 | 0.146822481 |
| HS_from_MQbrain_1462_r | 152.9071604 | 0.146822481 |
| HSbrain_705_r | 113.5172383 | 0.146822481 |
| HSbrain_624_l | 94.53967575 | 0.146822481 |
| HS_from_MQbrain_1250_r | 60.18097983 | 0.146822481 |
| Hsd_96_r | 5.165955532 | 0.146822481 |
| HS_from_PT_271_l | 2.99221888 | 0.146822481 |
| HS_from_PT_92_l | 303.3056853 | 0.152858542 |
| Hsd_38_r | 285.7326061 | 0.152858542 |
| HSbrain_266_l | 271.4320379 | 0.152858542 |
| HS_from_MQbrain_6_r | 264.8578117 | 0.152858542 |
| HS_from_PT_238_l | 251.4029293 | 0.152858542 |
| HSbrain_306_r | 226.3244274 | 0.152858542 |
| HS_from_MQbrain_707_l | 224.9895326 | 0.152858542 |
| HS_from_MQbrain_693_r | 197.7309441 | 0.152858542 |
| HSbrain_752_l | 191.3899851 | 0.152858542 |
| HS_from_MQbrain_969_r | 177.1203053 | 0.152858542 |
| HS_from_MQbrain_1072_r | 107.9850118 | 0.152858542 |
| HS_from_MQbrain_355_r | 91.78580467 | 0.152858542 |
| HSbrain_604_r | 57.67387084 | 0.152858542 |
| HS_from_MQbrain_153_l | 49.32700101 | 0.152858542 |
| Hsd_from_Mmd_280_l | 46.29319877 | 0.152858542 |
| HSbrain_22_l | 7.394968873 | 0.152858542 |
| HS_from_MQbrain_1127_r | 6.478685264 | 0.152858542 |
| HSbrain_695_r | 4.015688972 | 0.152858542 |
| HS_from_MQbrain_1817_l | 3.888478757 | 0.152858542 |
| HS_from_MQbrain_689_l | 2.726769607 | 0.152858542 |
| Hsd_from_Mmd_346_l | 2.586537564 | 0.152858542 |
| HSbrain_783_l | 345.0006901 | 0.158799149 |
| HSbrain_673_l | 313.971615 | 0.158799149 |
| HS_from_MQbrain_1018_l | 312.5603857 | 0.158799149 |
| HS_18_l | 244.6303758 | 0.158799149 |
| HS_from_MQbrain_1501_r | 242.0224401 | 0.158799149 |
| HS_from_PT_158_l | 238.7177237 | 0.158799149 |
| HSbrain_347_l | 211.107069 | 0.158799149 |
| HS_from_MQbrain_1204_r | 209.9240502 | 0.158799149 |
| HSbrain_248_l | 193.8830045 | 0.158799149 |
| HS_from_MQbrain_631_l | 184.728 | 0.158799149 |
| HS_from_PT_255_l | 179.6028399 | 0.158799149 |
| HS_from_MQbrain_1831_r | 179.507715 | 0.158799149 |
| HSbrain_121_l | 163.1114217 | 0.158799149 |
| HS_from_MQbrain_382_r | 108.3108904 | 0.158799149 |
| HS_from_MQbrain_625_l | 58.16380903 | 0.158799149 |
| HS_from_MQbrain_912_l | 42.57025194 | 0.158799149 |
| HSbrain_320_l | 8.739860007 | 0.158799149 |
| HS_from_MQbrain_675_l | 3.821332516 | 0.158799149 |
| HSbrain_413_l | 3.253659453 | 0.158799149 |
| HS_from_MM_35_l | 2.357250026 | 0.158799149 |
| HSbrain_402_l | 349.5145921 | 0.163891418 |
| HS_from_MQbrain_996_l | 302.9472283 | 0.163891418 |
| HS_from_MQbrain_824_r | 298.2022039 | 0.163891418 |
| HS_from_MQbrain_566_r | 291.1249796 | 0.163891418 |
| HSbrain_95_l | 253.768018 | 0.163891418 |
| HSbrain_680_l | 245.548398 | 0.163891418 |
| Hsd_from_Mmd_52_r | 243.7569251 | 0.163891418 |
| HS_from_MQbrain_1426_l | 242.4325995 | 0.163891418 |
| HS_from_MQbrain_1028_l | 239.0895004 | 0.163891418 |
| HSbrain_784_r | 234.6735882 | 0.163891418 |
| HS_from_MQbrain_650_r | 218.5666187 | 0.163891418 |
| HS_from_MQbrain_449_l | 213.7795306 | 0.163891418 |
| HSbrain_304_l | 204.8368425 | 0.163891418 |
| HS_from_MQbrain_1721_l | 197.7591475 | 0.163891418 |
| HS_from_MQbrain_936_r | 195.7842152 | 0.163891418 |
| Hsd_from_Mmd_94_l | 193.7657691 | 0.163891418 |
| HS_from_MQbrain_529_r | 190.4031906 | 0.163891418 |
| Hsd_108_r | 181.7633595 | 0.163891418 |
| HS_from_PT_234_l | 181.4676297 | 0.163891418 |
| HS_from_MQbrain_1223_l | 164.5470949 | 0.163891418 |
| HS_from_MQbrain_1724_l | 163.9516624 | 0.163891418 |
| HS_from_PT_196_r | 59.89771429 | 0.163891418 |
| HSbrain_598_r | 7.899656198 | 0.163891418 |
| HS_from_MM_218_l | 2.349494942 | 0.163891418 |
| HSbrain_467_l | 335.0856427 | 0.170271053 |
| HS_from_MQbrain_1846_r | 333.0341838 | 0.170271053 |
| HS_197_r | 299.8398179 | 0.170271053 |
| HS_212_r | 293.7948013 | 0.170271053 |
| HSbrain_53_l | 247.0890399 | 0.170271053 |
| HSbrain_701_l | 245.9313661 | 0.170271053 |
| HS_128_r | 214.1983377 | 0.170271053 |
| HSbrain_364_r | 211.3556115 | 0.170271053 |
| HS_from_MQbrain_281_l | 197.384259 | 0.170271053 |
| HS_from_MQbrain_1199_l | 177.5515382 | 0.170271053 |
| HSbrain_143_l | 173.8367679 | 0.170271053 |
| HSbrain_216_r | 164.5910344 | 0.170271053 |
| Hsd_from_Mmd_235_r | 145.886021 | 0.170271053 |
| HS_from_MQbrain_841_l | 143.150258 | 0.170271053 |
| Hsd_from_Mmd_367_l | 120.0545039 | 0.170271053 |
| HS_from_MQbrain_468_l | 113.3244619 | 0.170271053 |
| HS_from_MQbrain_1377_l | 110.9676187 | 0.170271053 |
| Hsd_from_Mmd_410_l | 94.2059708 | 0.170271053 |
| HS_192_r | 15.41603426 | 0.170271053 |
| HS_from_MQbrain_1893_r | 14.47922582 | 0.170271053 |
| HS_264_r | 7.905646906 | 0.170271053 |
| HS_from_MQbrain_603_r | 7.108558771 | 0.170271053 |
| HSbrain_726_l | 6.434813111 | 0.170271053 |
| Hsd_from_Mmd_35_l | 2.507108516 | 0.170271053 |
| Hsd_from_Mmd_344_r | 309.0726717 | 0.176183859 |
| HSbrain_626_l | 275.7891397 | 0.176183859 |
| HS_from_PT_27_r | 221.573516 | 0.176183859 |
| HSbrain_32_l | 216.2330231 | 0.176183859 |
| HS_186_r | 203.9246728 | 0.176183859 |
| HSbrain_771_l | 185.5150257 | 0.176183859 |
| Hsd_65_r | 184.3852713 | 0.176183859 |
| HS_from_MQbrain_135_l | 155.6010506 | 0.176183859 |
| HS_from_MQbrain_628_l | 105.1976832 | 0.176183859 |
| HSbrain_707_r | 70.97237044 | 0.176183859 |
| HS_from_MQbrain_984_l | 36.17853558 | 0.176183859 |
| HS_204_r | 14.93137289 | 0.176183859 |
| HS_130_l | 10.32443146 | 0.176183859 |
| HS_from_MQbrain_365_l | 6.941331531 | 0.176183859 |
| HS_from_MQbrain_855_r | 6.514975985 | 0.176183859 |
| HS_from_MQbrain_314_l | 2.61540593 | 0.176183859 |
| HS_from_MM_84_l | 2.359765653 | 0.176183859 |
| HS_from_MQbrain_1349_l | 328.4193266 | 0.182758186 |
| HS_from_MQbrain_162_r | 314.656125 | 0.182758186 |
| HS_from_MQbrain_850_l | 288.5662281 | 0.182758186 |
| HS_from_MQbrain_1509_r | 283.4583708 | 0.182758186 |
| HS_from_MQbrain_1380_r | 253.5211282 | 0.182758186 |
| HSbrain_388_r | 213.3920423 | 0.182758186 |
| HS_from_MM_6_l | 190.1275732 | 0.182758186 |
| HS_from_MQbrain_881_l | 189.779606 | 0.182758186 |
| HS_from_MM_160_l | 137.0116145 | 0.182758186 |
| HS_from_MM_124_r | 124.074717 | 0.182758186 |
| Hsd_from_Mmd_187_r | 109.5214584 | 0.182758186 |
| HS_from_MQbrain_361_l | 108.0467095 | 0.182758186 |
| HS_from_MM_149_r | 75.91523091 | 0.182758186 |
| Hsd_from_Mmd_68_l | 61.78328125 | 0.182758186 |
| HS_from_MQbrain_676_r | 52.19716118 | 0.182758186 |
| HS_from_MQbrain_1883_l | 21.12907054 | 0.182758186 |
| HSbrain_706_l | 15.17246512 | 0.182758186 |
| HS_from_MQbrain_1576_r | 8.509211792 | 0.182758186 |
| HS_132_l | 7.348116533 | 0.182758186 |
| HS_from_MQbrain_1137_l | 2.48333526 | 0.182758186 |
| HS_from_MQbrain_1596_r | 435.4766611 | 0.188867139 |
| HS_from_MQbrain_1073_r | 319.8894959 | 0.188867139 |
| HS_from_MQbrain_487_r | 282.9624273 | 0.188867139 |
| HSbrain_210_l | 218.1425564 | 0.188867139 |
| HSbrain_627_r | 214.4670798 | 0.188867139 |
| HS_77_l | 209.1463931 | 0.188867139 |
| HS_from_MM_182_r | 208.0844095 | 0.188867139 |
| HS_from_MQbrain_1208_l | 108.8734707 | 0.188867139 |
| HS_from_MM_86_l | 89.48891073 | 0.188867139 |
| HSbrain_553_l | 69.73500772 | 0.188867139 |
| HS_from_PT_328_l | 64.37288073 | 0.188867139 |
| HS_from_MQbrain_1734_l | 57.12642239 | 0.188867139 |
| HSbrain_370_r | 45.65991936 | 0.188867139 |
| HS_from_MM_298_l | 41.95783366 | 0.188867139 |
| HS_from_MQbrain_1234_l | 18.99466667 | 0.188867139 |
| HS_from_PT_247_l | 17.14193784 | 0.188867139 |
| HSbrain_242_l | 9.140095581 | 0.188867139 |
| Hsd_from_Mmd_314_r | 6.401270974 | 0.188867139 |
| HS_from_MQbrain_768_l | 3.670091279 | 0.188867139 |
| HSbrain_270_l | 3.298294118 | 0.188867139 |
| HSbrain_810_l | 274.8805235 | 0.19639408 |
| HS_124_l | 273.022187 | 0.19639408 |
| HS_198_l | 239.6726 | 0.19639408 |
| HSbrain_616_r | 195.4635888 | 0.19639408 |
| HS_from_PT_204_r | 191.0832874 | 0.19639408 |
| HS_from_MQbrain_406_r | 189.233991 | 0.19639408 |
| HS_from_MQbrain_383_l | 188.3158756 | 0.19639408 |
| Hsd_19_l | 62.00218054 | 0.19639408 |
| HSbrain_295_r | 60.35523089 | 0.19639408 |
| HS_from_PT_130_r | 49.02454902 | 0.19639408 |
| HS_from_MQbrain_389_l | 10.36562841 | 0.19639408 |

¹Cut off Signal/background >2
²Cut off adj p < 0.2

TABLE 9b
Small RNAs recovered from faeces and differentially expressed in
adenoma vs control tissue: hairpin with lowest expression

| | 2^{nd} arm | adenoma vs control |
|---|---|---|
| Systematic name | signal/background¹ | (adj p)² |
| HSbrain_70_r | 228.1310199 | 0.19639408 |
| HS_from_MQbrain_648_l | 158.7568209 | 0.19639408 |
| HSbrain_658_l | 129.0251642 | 0.19639408 |
| HS_203_l | 94.83523983 | 0.19639408 |
| Hsd_from_Mmd_399_r | 81.10162309 | 0.19639408 |
| Hsd_from_Mmd_326_l | 36.19385915 | 0.19639408 |
| HSbrain_180_l | 9.145529202 | 0.19639408 |
| HS_from_MQbrain_84_r | 9.015220167 | 0.19639408 |
| HS_from_MQbrain_1883_r | 6.77899065 | 0.19639408 |
| HSbrain_433_r | 6.513823308 | 0.19639408 |
| HS_from_MQbrain_1783_r | 5.660060677 | 0.19639408 |
| HS_from_MQbrain_1671_l | 2.718046822 | 0.19639408 |
| HSbrain_403_l | 130.6652289 | 0.188867139 |
| HS_from_MQbrain_1760_r | 68.93637025 | 0.188867139 |
| Hsd_from_Mmd_120_l | 6.012536709 | 0.188867139 |
| HS_from_MM_331_r | 5.344166534 | 0.188867139 |
| Hsd_from_Mmd_325_r | 2.644428741 | 0.188867139 |
| HS_from_PT_271_r | 2.554264498 | 0.188867139 |
| HS_from_MQbrain_999_r | 2.263925063 | 0.188867139 |
| HS_from_PT_3_l | 2.115103843 | 0.188867139 |
| HS_from_PT_161_l | 171.1553574 | 0.182758186 |
| HS_from_MQbrain_694_r | 5.319743993 | 0.182758186 |
| HS_from_MQbrain_635_l | 3.030934579 | 0.182758186 |
| Hsd_from_Mmd_333_l | 2.003374918 | 0.182758186 |
| Hsd_from_Mmd_92_l | 116.8521775 | 0.176183859 |
| HS_from_MQbrain_1566_r | 28.34610003 | 0.176183859 |
| HS_from_MM_123_l | 25.84005888 | 0.176183859 |
| HS_from_MQbrain_1565_l | 11.415781 | 0.176183859 |
| Hsd_from_Mmd_123_r | 3.109340734 | 0.176183859 |
| Hsd_from_Mmd_328_l | 2.054062827 | 0.176183859 |
| HSbrain_745_l | 199.5248523 | 0.170271053 |
| HS_from_MM_304_l | 44.91042461 | 0.170271053 |
| Hsd_47_r | 15.14357414 | 0.170271053 |
| HS_78_r | 3.599814983 | 0.170271053 |
| HS_from_PT_280_l | 230.7126831 | 0.163891418 |
| HS_from_MQbrain_1090_r | 148.6783798 | 0.163891418 |
| Hsd_84_r | 14.08450328 | 0.163891418 |
| HS_from_MQbrain_941_r | 2.300164622 | 0.163891418 |
| HS_from_MM_340_r | 2.08874036 | 0.163891418 |
| HS_from_MQbrain_6_l | 224.1267133 | 0.158799149 |
| HS_from_MQbrain_414_l | 178.1375421 | 0.158799149 |
| HSbrain_653_l | 40.34177825 | 0.158799149 |
| HS_from_MQbrain_140_l | 5.24618389 | 0.158799149 |
| HS_from_MQbrain_68_r | 3.894404987 | 0.158799149 |
| Hsd_67_r | 2.507940384 | 0.158799149 |
| HS_from_MQbrain_340_l | 2.901321612 | 0.152858542 |
| HS_from_MQbrain_1759_l | 210.0656412 | 0.146822481 |
| HS_from_MQbrain_260_r | 190.0240714 | 0.146822481 |
| HS_from_PT_284_r | 27.92056913 | 0.146822481 |
| HS_268_l | 6.398871252 | 0.146822481 |
| HSbrain_126_r | 185.8597074 | 0.140323812 |
| HS_from_MQbrain_266_r | 7.219897398 | 0.140323812 |
| Hsd_from_Mmd_291_l | 44.20392105 | 0.134970683 |
| HS_from_MQbrain_1376_r | 12.27563489 | 0.134970683 |
| Hsd_from_Mmd_388_l | 4.044215903 | 0.134970683 |
| HSbrain_267_r | 187.7274337 | 0.129399391 |
| HS_from_MQbrain_466_l | 173.774731 | 0.129399391 |
| HS_from_MQbrain_1712_r | 148.9640462 | 0.129399391 |
| HS_from_MQbrain_1722_l | 110.8769733 | 0.129399391 |
| HS_from_MQbrain_137_r | 9.243852235 | 0.129399391 |
| HS_from_MQbrain_1377_r | 2.685212414 | 0.129399391 |
| Hsd_from_Mmd_183_r | 148.0528441 | 0.12422104 |
| HS_135_l | 131.4469186 | 0.12422104 |
| HS_from_MQbrain_485_l | 8.243037088 | 0.12422104 |
| HS_from_PT_237_l | 3.488690264 | 0.12422104 |
| HSbrain_383_l | 11.26061345 | 0.11858389 |
| Hsd_60_r | 5.759084269 | 0.11858389 |
| HS_from_MQbrain_1051_r | 2.890006235 | 0.11858389 |
| HS_106_r | 2.537522063 | 0.11858389 |
| HS_from_MM_251_r | 168.6680712 | 0.113099484 |
| HS_204_l | 14.86560477 | 0.113099484 |
| HS_from_MQbrain_824_l | 2.021767126 | 0.113099484 |
| HS_164_l | 113.9895739 | 0.108118292 |
| HSbrain_299_r | 26.59238777 | 0.108118292 |
| HS_from_MQbrain_594_r | 2.753205678 | 0.108118292 |
| HS_from_MM_124_l | 2.285310959 | 0.108118292 |
| Hsd_111_r | 204.558847 | 0.103393572 |
| HS_from_MQbrain_751_l | 171.7203726 | 0.103393572 |
| HSbrain_319_l | 27.65786021 | 0.103393572 |
| HS_from_MQbrain_1633_r | 10.30485548 | 0.103393572 |
| HS_from_MQbrain_1410_l | 47.23710024 | 0.098921026 |
| HS_from_MQbrain_1139_r | 27.78815761 | 0.098921026 |
| HSbrain_209_l | 5.831860813 | 0.095394629 |
| HS_from_MQbrain_932_l | 2.123754543 | 0.095394629 |
| HS_from_MQbrain_1678_r | 148.7689177 | 0.09152926 |
| Hsd_from_Mmd_371_r | 131.2157622 | 0.09152926 |
| HS_from_MQbrain_1031_l | 46.34701799 | 0.09152926 |
| HS_from_MQbrain_1602_l | 35.32112193 | 0.09152926 |
| HS_from_MQbrain_770_r | 5.072552769 | 0.09152926 |
| HSbrain_619_r | 4.2712614 | 0.09152926 |
| HSbrain_19_l | 3.145160187 | 0.09152926 |
| HS_from_MQbrain_1437_l | 2.717424689 | 0.09152926 |
| HS_from_PT_126_l | 6.759793869 | 0.088143181 |
| HS_11_l | 6.010655386 | 0.088143181 |
| HS_from_MM_202_l | 65.31801875 | 0.083930001 |
| HS_from_MQbrain_814_l | 11.67524525 | 0.083930001 |
| HS_from_MQbrain_597_r | 4.765274065 | 0.083930001 |
| HS_from_MQbrain_1706_l | 213.0219833 | 0.079954597 |
| HS_from_MQbrain_1665_r | 87.0752453 | 0.079954597 |
| HS_from_MQbrain_253_r | 75.90944638 | 0.072349461 |
| HS_81_r | 55.61137135 | 0.072349461 |
| HS_from_MQbrain_1748_r | 19.48179132 | 0.072349461 |
| HSbrain_635_l | 6.819097351 | 0.072349461 |
| HS_from_MQbrain_1268_l | 5.668043125 | 0.072349461 |
| HS_from_PT_235_l | 208.8800625 | 0.069749265 |
| HS_from_MM_47_l | 2.919273938 | 0.067503134 |
| HS_from_MQbrain_1696_l | 154.0498567 | 0.064770288 |
| HS_from_MQbrain_1450_r | 130.7495827 | 0.064770288 |
| HSbrain_776_l | 114.3914887 | 0.064770288 |
| HS_from_MQbrain_435_r | 2.200043662 | 0.064770288 |
| HS_from_MM_6_r | 2.160347119 | 0.064770288 |
| HS_from_PT_41_r | 2.157415789 | 0.064770288 |
| Hsd_from_Mmd_439_l | 80.82870283 | 0.061046963 |
| HS_from_PT_340_l | 17.88452337 | 0.061046963 |
| HS_from_MQbrain_1500_l | 15.2774537 | 0.061046963 |
| HS_from_MQbrain_1412_l | 4.664856717 | 0.061046963 |
| HS_from_PT_242_l | 2.025087308 | 0.061046963 |
| HS_156_l | 16.10230692 | 0.058253679 |
| HS_from_MQbrain_1794_l | 34.34325904 | 0.053159859 |
| HS_from_MQbrain_1771_l | 188.7899392 | 0.050130838 |
| HS_from_MQbrain_691_l | 30.90923016 | 0.050130838 |
| Hsd_65_l | 5.317412014 | 0.050130838 |
| HS_from_MQbrain_842_l | 2.595893731 | 0.050130838 |
| HS_from_MQbrain_1752_l | 22.86665735 | 0.048378078 |
| HS_from_MM_289_l | 40.04595101 | 0.042549287 |
| HS_177_l | 39.31124871 | 0.042549287 |
| HS_from_PT_91_r | 11.52114628 | 0.042549287 |
| HS_from_MQbrain_1087_l | 8.47223421 | 0.038273758 |
| Hsd_from_Mmd_410_r | 2.908576361 | 0.036099692 |
| Hsd_105_l | 5.399875994 | 0.03224042 |
| HSbrain_296_l | 2.843440689 | 0.02997784 |
| Hsd_from_Mmd_219_r | 42.949196 | 0.024762366 |
| Hsd_from_Mmd_519_l | 32.14358853 | 0.024762366 |
| Hsd_26_l | 5.446771505 | 0.024762366 |
| HSbrain_778_r | 85.0377047 | 0.023736962 |
| HSbrain_794_r | 74.07648688 | 0.023736962 |
| HSbrain_703_r | 11.82131557 | 0.022745616 |
| HSbrain_362_r | 9.722304379 | 0.019454312 |
| HSbrain_268_r | 158.6132777 | 0.018600058 |
| HSbrain_253_l | 11.33164065 | 0.018600058 |
| HS_from_MQbrain_825_r | 3.634806505 | 0.011699489 |
| HS_from_MQbrain_1628_r | 3.67815753 | 0.008946182 |
| HS_from_MQbrain_1462_l | 135.9829616 | 0.008573112 |
| HS_from_MQbrain_1317_r | 11.20285772 | 0.00676609 |
| HS_from_MQbrain_1311_r | 2.389862917 | 0.00676609 |
| HS_from_MQbrain_1070_r | 19.32265622 | 0.004799641 |
| HS_21_r | 216.9668884 | 0.000489293 |

¹Cut off Signal/background > 2
²Cut off adj p < 0.2

TABLE 10a
Small RNAs recovered from faeces and differentially expressed in
adenocarcinoma vs normal tissue: hairpin arm with highest expression

| | | carcinoma |
|---|---|---|
| | 1st arm signal/ | vs control |
| Systematic name | background¹ | (adj p)² |
| HS_from_MQbrain_1393_r | 223.7656716 | 0.016433541 |
| HS_from_MM_334_l | 165.3471372 | 0.016433541 |
| Hsd_from_Mmd_473_l | 15.1413037 | 0.019274533 |
| HSbrain_377_r | 2.687263122 | 0.019274533 |
| HSbrain_617_l | 59.52352443 | 0.022921248 |
| HS_from_MM_178_r | 86.39079121 | 0.030988376 |
| HS_from_MQbrain_1056_l | 71.54390765 | 0.031989408 |
| HS_from_MQbrain_1823_r | 44.43291919 | 0.0342406 |
| HS_from_MM_39_l | 15.46864042 | 0.0342406 |
| Hsd_102_l | 175.5645249 | 0.035303066 |
| HS_from_PT_190_r | 138.4453126 | 0.045228199 |
| HSbrain_171_l | 255.6856718 | 0.067337504 |
| HS_262_l | 14.64955868 | 0.067337504 |
| HS_from_MQbrain_343_l | 214.8585663 | 0.07566513 |
| HS_from_MQbrain_1169_l | 105.7095098 | 0.084904114 |
| HSbrain_629_l | 254.3371274 | 0.086503988 |
| HSbrain_597_r | 208.6310714 | 0.086503988 |
| HS_from_MQbrain_350_l | 189.461419 | 0.086503988 |
| HSbrain_12_l | 53.76557022 | 0.086503988 |
| HS_from_MQbrain_2380_r | 187.2947993 | 0.090694253 |
| HS_255_l | 161.1405598 | 0.090694253 |
| HS_from_MQbrain_1128_l | 158.837926 | 0.090694253 |
| HS_from_MQbrain_349_l | 135.3020631 | 0.090694253 |
| Hsd_from_Mmd_328_r | 117.324613 | 0.090694253 |
| HSbrain_753_r | 41.77257724 | 0.090694253 |
| HS_from_MQbrain_55_l | 15.80840103 | 0.090694253 |
| HSbrain_177_r | 132.197755 | 0.104922786 |
| HSbrain_446_l | 14.33402718 | 0.104922786 |
| HSbrain_483_l | 220.2067855 | 0.110995143 |
| HS_from_MQbrain_128_r | 194.6783323 | 0.110995143 |
| HSbrain_709_l | 274.820351 | 0.120015634 |
| HS_from_MM_309_r | 208.0343975 | 0.120015634 |
| HS_217_l | 228.449758 | 0.120843678 |
| HS_from_MQbrain_930_r | 167.8363796 | 0.120843678 |
| HS_141_l | 141.8565157 | 0.120843678 |
| HS_from_MQbrain_1657_r | 116.3148049 | 0.120843678 |
| HS_from_MQbrain_769_r | 87.406602 | 0.120843678 |
| HS_from_PT_257_r | 27.728139 | 0.120843678 |
| HS_from_PT_201_r | 2.367251208 | 0.120843678 |
| HSbrain_801_r | 195.7930985 | 0.149145996 |
| HS_from_MQbrain_1624_l | 192.3884805 | 0.149145996 |
| HSbrain_158_l | 169.343928 | 0.149145996 |
| HSbrain_1_l | 76.8373873 | 0.149145996 |
| Hsd_18_l | 237.5322474 | 0.159103307 |
| HSbrain_261_l | 83.18937251 | 0.159103307 |
| HS_from_MQbrain_1459_l | 257.1079597 | 0.166408872 |
| HSbrain_458_l | 77.3089971 | 0.166408872 |
| HS_from_MM_339_r | 327.2871875 | 0.179914563 |
| HS_304_r | 244.034349 | 0.179914563 |
| HS_from_MQbrain_1513_r | 128.9407151 | 0.19263196 |
| Hsd_from_Mmd_298_r | 48.27315482 | 0.19263196 |

¹Cut off Signal/background > 2
²Cut off adj p < 0.2

TABLE 10b
Small RNAs recovered from faeces and differentially expressed in
adenocarcinoma vs normal tissue: hairpin arm with lowest expression

| | | carcinoma |
|---|---|---|
| | 2^{nd} arm signal/ | vs control |
| systematic name | background¹ | (adj p)² |
| HS_21_r | 216.9668884 | 0.099652833 |
| Hsd_111_r | 204.558847 | 0.149145996 |
| HS_from_MQbrain_1696_l | 154.0498567 | 0.126655157 |
| HS_from_MM_289_l | 40.04595101 | 0.104922786 |
| HSbrain_299_r | 26.59238777 | 0.159103307 |
| HS_from_PT_340_l | 17.88452337 | 0.090694253 |
| HS_156_l | 16.10230692 | 0.120015634 |
| HS_from_MQbrain_1317_r | 11.20285772 | 0.008895418 |

¹Cut off Signal/background > 2
²Cut off adj p < 0.2

TABLE 11a
Small RNAs recovered from faeces and differentially expressed in
adenocarcinoma vs adenoma: hairpin arm with highest expression

| | | carcinoma vs |
|---|---|---|
| | 1^{st} arm signal/ | adenoma |
| Systematic name | background¹ | (adj p)² |
| HS_from_MM_118_r | 70.86890311 | 0.012664553 |
| HS_169_l | 153.6041594 | 0.014416465 |
| HS_299_l | 175.2031924 | 0.016786613 |
| HS_from_MQbrain_1137_l | 2.48333526 | 0.016904198 |
| HS_from_MQbrain_1082_l | 265.0804535 | 0.018319912 |
| HS_from_MQbrain_1522_r | 112.3011721 | 0.020545593 |
| HSbrain_306_r | 226.3244274 | 0.023919533 |
| HS_from_MQbrain_629_l | 195.1102679 | 0.026776445 |
| HS_from_MQbrain_1456_l | 199.1014452 | 0.028202169 |
| HS_from_MQbrain_828_l | 223.2821181 | 0.032034428 |
| HS_from_PT_40_r | 19.96645961 | 0.032034428 |
| HSbrain_384_l | 4.544181943 | 0.034662803 |
| HSbrain_1425_l | 283.5640729 | 0.035941334 |
| HS_from_MQbrain_1750_r | 217.7023298 | 0.037874034 |
| HS_from_MQbrain_984_l | 36.17853558 | 0.039250091 |
| HS_from_MQbrain_686_r | 259.8101317 | 0.044599949 |
| HS_141_l | 141.8565157 | 0.044599949 |
| HS_from_MQbrain_603_r | 7.108558771 | 0.044599949 |
| HS_218_r | 235.8613357 | 0.045945376 |
| HS_from_MM_169_r | 8.697594288 | 0.048205391 |
| HS_217_l | 228.449758 | 0.050135128 |
| HSbrain_226_r | 225.405043 | 0.050135128 |
| HSbrain_187_r | 179.7554723 | 0.050135128 |
| HSbrain_538_r | 3.973394819 | 0.055185029 |
| HS_from_MM_218_l | 2.349494942 | 0.055185029 |
| HS_from_MQbrain_1340_l | 157.6981977 | 0.058254254 |
| HSbrain_337_l | 85.40127884 | 0.059897432 |
| HSbrain_791_l | 10.8880558 | 0.059897432 |
| HSbrain_25_r | 68.45071483 | 0.062381992 |
| HS_from_MQbrain_330_r | 236.6286556 | 0.0659723 |
| Hsd_from_Mmd_391_l | 43.63503635 | 0.070590475 |
| Hsd_from_Mmd_237_l | 306.9716516 | 0.071451516 |
| HS_from_MQbrain_93_r | 290.7380082 | 0.071451516 |
| HS_from_MQbrain_1832_l | 275.5897539 | 0.071451516 |
| HSbrain_468_r | 8.648929144 | 0.071451516 |
| HSbrain_182_r | 228.0472357 | 0.072275086 |
| HSbrain_2_l | 49.67189807 | 0.072275086 |
| HS_from_MQbrain_869_l | 197.9550289 | 0.072654718 |
| HSbrain_1406_l | 17.08291783 | 0.072654718 |
| HS_from_MQbrain_74_r | 225.8922409 | 0.074901054 |
| HSbrain_419_l | 219.8793357 | 0.074901054 |
| HS_219_l | 254.6760586 | 0.075028175 |
| HS_from_MM_103_l | 246.9217731 | 0.075028175 |
| HS_from_MQbrain_1001_r | 223.8116976 | 0.075028175 |
| HS_from_MQbrain_470_l | 131.1632076 | 0.075028175 |
| Hsd_from_Mmd_189_l | 180.4050764 | 0.07641095 |
| Hsd_from_Mmd_527_r | 126.3935973 | 0.07641095 |
| HS_119_l | 233.7784735 | 0.07715065 |
| HSbrain_204_r | 217.0811451 | 0.07715065 |
| HS_118_l | 214.1827525 | 0.07715065 |
| HS_from_MM_275_r | 8.801848936 | 0.07715065 |
| HS_from_MM_342_r | 5.590510164 | 0.07715065 |
| HSbrain_442_r | 18.08359556 | 0.07965977 |
| HSbrain_361_l | 234.5281831 | 0.080676612 |
| HSbrain_328_l | 280.7719926 | 0.08237725 |
| HS_from_PT_27_r | 221.573516 | 0.08237725 |
| HSbrain_799_r | 3.32764451 | 0.08237725 |
| HS_from_MQbrain_1569_l | 191.4989985 | 0.083833911 |
| HS_from_MQbrain_634_r | 40.90596624 | 0.083833911 |
| HS_from_MQbrain_1675_r | 288.3970715 | 0.084885584 |
| HSbrain_95_l | 253.768018 | 0.084885584 |
| HSbrain_6_r | 244.8860331 | 0.084885584 |
| HS_from_MQbrain_1623_r | 244.0389624 | 0.084885584 |
| Hsd_from_Mmd_261_l | 184.5887939 | 0.084885584 |
| HS_from_MQbrain_1443_r | 179.1434855 | 0.084885584 |
| HSbrain_389_l | 9.742590244 | 0.084885584 |
| Hsd_106_l | 9.004764262 | 0.084885584 |
| HS_from_MQbrain_1294_l | 2.290891026 | 0.084885584 |
| HS_from_MQbrain_928_r | 318.1021923 | 0.085697388 |
| HS_from_MQbrain_965_l | 235.4627068 | 0.085697388 |
| HSbrain_764_r | 117.9645256 | 0.085697388 |
| Hsd_101_l | 3.714373078 | 0.085697388 |
| HS_49_r | 8.969465473 | 0.088269125 |
| HS_from_MQbrain_1355_r | 309.6691714 | 0.089491072 |
| HS_from_MQbrain_597_l | 8.606774472 | 0.089491072 |
| HS_from_MQbrain_392_l | 254.8014108 | 0.091337111 |
| HS_from_MQbrain_765_l | 254.6466837 | 0.091337111 |
| HS_from_MQbrain_1771_r | 247.8706875 | 0.091337111 |
| HSbrain_594_l | 186.9175668 | 0.091337111 |
| HS_from_MQbrain_631_l | 184.728 | 0.091337111 |
| HS_from_MM_219_l | 47.65091531 | 0.091337111 |
| HSbrain_540_r | 19.28862768 | 0.091337111 |
| HS_from_PT_282_l | 11.85767781 | 0.091337111 |
| HS_from_MQbrain_131_r | 202.0595323 | 0.092780591 |
| Hsd_from_Mmd_491_r | 9.356107716 | 0.092780591 |
| HS_from_MQbrain_191_l | 203.1756555 | 0.094556199 |
| Hsd_from_Mmd_330_l | 200.078931 | 0.094556199 |
| HSbrain_591_l | 185.5052057 | 0.094556199 |
| HSbrain_123_l | 203.4486322 | 0.096132958 |
| HS_from_MQbrain_1793_r | 103.5900968 | 0.096132958 |
| HSbrain_532_l | 11.20284123 | 0.096132958 |
| HSbrain_316_l | 339.4367001 | 0.098301345 |
| HS_from_MQbrain_1183_l | 198.4935006 | 0.098301345 |
| HS_from_MQbrain_1893_r | 14.47922582 | 0.098301345 |
| HS_from_MQbrain_1031_r | 263.8971038 | 0.099488281 |
| HSbrain_719_l | 9.057424006 | 0.099488281 |
| HS_from_MQbrain_1840_l | 4.101477435 | 0.099488281 |
| Hsd_70_l | 292.1907862 | 0.101009439 |
| HS_from_MQbrain_1827_l | 289.495665 | 0.101009439 |
| HSbrain_703_l | 247.3420423 | 0.101009439 |
| HSbrain_14_r | 99.51998497 | 0.101009439 |
| HS_from_MQbrain_1806_l | 4.715008642 | 0.101009439 |
| Hsd_from_Mmd_346_l | 2.586537564 | 0.101009439 |
| HS_from_MM_84_l | 2.359765653 | 0.101009439 |
| HS_from_MQbrain_628_l | 105.1976832 | 0.102601153 |
| HS_from_MQbrain_722_r | 230.0329269 | 0.104515233 |
| HS_from_MQbrain_433_l | 186.8433165 | 0.104515233 |
| HS_from_MQbrain_1374_l | 176.8739274 | 0.104515233 |
| HS_from_MQbrain_534_r | 240.0925796 | 0.105993846 |
| HS_from_MQbrain_22_r | 187.1051151 | 0.105993846 |
| HS_from_MQbrain_1127_r | 6.478685264 | 0.105993846 |
| HS_from_MQbrain_996_l | 302.9472283 | 0.107794024 |
| HS_from_MQbrain_574_l | 287.7017103 | 0.107794024 |
| HS_from_MQbrain_951_l | 230.432556 | 0.107794024 |
| HS_from_MQbrain_921_l | 192.1335465 | 0.107794024 |
| HS_from_MQbrain_1541_l | 32.58806331 | 0.107794024 |
| Hsd_from_Mmd_52_r | 243.7569251 | 0.11066006 |
| Hsd_from_Mmd_266_r | 239.3412808 | 0.11066006 |
| HSbrain_677_l | 223.7296488 | 0.11066006 |
| HS_from_MM_86_l | 89.48891073 | 0.11066006 |
| HSbrain_588_l | 269.5888825 | 0.113095726 |
| HSbrain_426_r | 262.208223 | 0.113095726 |
| HSbrain_440_l | 231.4604735 | 0.113095726 |
| HSbrain_50_l | 203.7449536 | 0.113095726 |
| HS_from_MQbrain_29_l | 198.1116988 | 0.113095726 |
| HS_from_MQbrain_777_r | 120.2896834 | 0.113095726 |
| HS_from_MQbrain_1164_r | 265.941125 | 0.114595796 |
| HS_from_MQbrain_56_l | 221.5946492 | 0.114595796 |
| HS_102_l | 205.6195359 | 0.114595796 |
| HS_from_MM_276_l | 21.75172107 | 0.114595796 |
| HS_from_MQbrain_1817_l | 3.888478757 | 0.114595796 |
| HS_from_MQbrain_609_l | 213.1909341 | 0.115928328 |
| HS_from_MQbrain_501_l | 152.6321911 | 0.115928328 |
| HS_from_MQbrain_1637_r | 147.2561547 | 0.115928328 |
| HSbrain_26_r | 3.807676617 | 0.115928328 |
| HS_from_MM_208_r | 241.9491489 | 0.117828853 |
| HS_246_l | 233.9810458 | 0.117828853 |
| HS_from_MM_34_l | 7.978839103 | 0.117828853 |
| HS_from_MQbrain_974_l | 247.9593335 | 0.119559457 |
| HS_from_PT_60_l | 241.2340994 | 0.119559457 |
| HSbrain_708_l | 229.246264 | 0.119559457 |
| HSbrain_606_l | 181.1992813 | 0.119559457 |
| HS_from_MQbrain_1632_l | 234.3869839 | 0.121604253 |
| HSbrain_305_r | 219.5650075 | 0.121604253 |
| HS_from_MQbrain_648_r | 216.5701764 | 0.121604253 |
| HS_from_MQbrain_161_r | 6.387946973 | 0.121604253 |
| HSbrain_402_l | 349.5145921 | 0.12300785 |
| HS_from_MQbrain_1075_l | 258.9475578 | 0.12300785 |
| HS_from_MQbrain_1261_r | 243.9532889 | 0.12300785 |
| HS_from_MQbrain_266_l | 243.4504792 | 0.12300785 |
| HS_from_MQbrain_340_r | 243.3848161 | 0.12300785 |
| HSbrain_256_r | 12.675 | 0.12300785 |
| Hsd_from_Mmd_314_r | 6.401270974 | 0.12300785 |
| HS_from_MQbrain_210_r | 204.1332639 | 0.124263194 |
| HS_from_MQbrain_1097_r | 128.6204149 | 0.124263194 |
| Hsd_from_Mmd_154_l | 2.836322667 | 0.124263194 |
| HSbrain_771_l | 185.5150257 | 0.126757752 |
| HS_from_MQbrain_314_l | 2.61540593 | 0.126757752 |
| HS_from_MQbrain_822_l | 2.343923008 | 0.126757752 |
| HSbrain_393_r | 336.1272016 | 0.129555956 |
| HS_from_MQbrain_1102_l | 303.4601583 | 0.129555956 |
| HS_from_MQbrain_1413_l | 204.3272671 | 0.129555956 |
| HS_from_MQbrain_453_l | 57.09533664 | 0.129555956 |
| HSbrain_783_l | 345.0006901 | 0.130803986 |
| HS_from_MQbrain_672_l | 249.9938404 | 0.130803986 |
| Hsd_from_Mmd_94_l | 193.7657691 | 0.130803986 |
| HS_from_MQbrain_216_l | 182.4516418 | 0.130803986 |
| HS_from_PT_313_l | 113.037452 | 0.130803986 |
| HSbrain_380_r | 54.63237608 | 0.130803986 |
| HS_from_MQbrain_868_l | 260.4645119 | 0.132596379 |
| HS_101_l | 239.0332153 | 0.132596379 |
| HS_from_MQbrain_715_l | 216.9853807 | 0.132596379 |
| HSbrain_691_l | 201.4670994 | 0.132596379 |
| HS_from_MQbrain_1100_l | 165.2906308 | 0.132596379 |
| HSbrain_726_l | 6.434813111 | 0.132596379 |
| HSbrain_484_l | 270.1785451 | 0.134691663 |
| Hsd_42_r | 260.2142458 | 0.134691663 |
| HSbrain_243_l | 182.2549801 | 0.134691663 |
| HS_62_l | 66.38174565 | 0.134691663 |
| Hsd_7_r | 23.39907905 | 0.134691663 |
| HS_from_PT_44_l | 269.9446191 | 0.136637202 |
| HSbrain_970_r | 2.440271917 | 0.136637202 |
| HSbrain_250_l | 313.5276305 | 0.138217661 |
| HS_from_MQbrain_1824_l | 292.4713609 | 0.138217661 |
| HS_from_MQbrain_70_l | 285.3653998 | 0.138217661 |
| HS_from_MQbrain_1043_r | 281.5762963 | 0.138217661 |
| Hsd_69_l | 273.3125807 | 0.138217661 |
| HS_from_MQbrain_315_l | 85.77155971 | 0.138217661 |
| HS_from_MQbrain_1836_l | 6.267799948 | 0.138217661 |
| HS_from_MQbrain_886_r | 310.0903943 | 0.139019426 |
| HS_from_MQbrain_1756_l | 252.4873393 | 0.139019426 |
| HS_from_MQbrain_399_l | 238.3085443 | 0.139019426 |
| HSbrain_420_r | 228.3614012 | 0.139019426 |
| HS_from_MQbrain_1493_r | 207.5291507 | 0.139019426 |
| HS_from_MQbrain_90_r | 178.3364821 | 0.139019426 |
| HS_277_l | 168.4332786 | 0.139019426 |
| Hsd_13_l | 130.4556358 | 0.139019426 |
| HSbrain_3_l | 51.24508411 | 0.139019426 |
| HS_98_r | 330.803132 | 0.141416815 |
| HS_39_r | 320.0335858 | 0.141416815 |
| HS_from_MQbrain_1331_r | 233.5757256 | 0.141416815 |
| HS_from_MQbrain_845_r | 228.2389891 | 0.141416815 |
| HS_from_MQbrain_824_r | 298.2022039 | 0.14474535 |
| HS_from_MQbrain_914_l | 266.9248651 | 0.14474535 |
| HSbrain_369_r | 159.7073641 | 0.14474535 |
| HSbrain_82_r | 99.35128086 | 0.14474535 |
| HS_from_MQbrain_1227_l | 243.816199 | 0.145370181 |
| HS_from_MQbrain_1810_l | 218.9019542 | 0.145370181 |
| HSbrain_620_l | 216.6725849 | 0.145370181 |
| HS_from_MQbrain_180_r | 210.3209412 | 0.145370181 |
| HS_from_MQbrain_1204_r | 209.9240502 | 0.145370181 |
| HS_54_l | 185.9367326 | 0.145370181 |
| HSbrain_716_l | 180.5676912 | 0.145370181 |
| HSbrain_117_r | 100.8429431 | 0.145370181 |
| HSbrain_214_l | 19.36197308 | 0.145370181 |
| Hsd_from_Mmd_246_r | 309.4213909 | 0.146325218 |
| HSbrain_307_r | 231.9591602 | 0.146325218 |
| HS_from_MQbrain_973_l | 229.669574 | 0.146325218 |
| HS_306_l | 211.5536628 | 0.146325218 |
| HS_133_l | 183.5586928 | 0.146325218 |
| HS_from_MM_199_l | 170.7212373 | 0.146325218 |
| HS_from_MQbrain_606_r | 105.4640055 | 0.146325218 |
| HS_from_MQbrain_671_r | 263.6821505 | 0.148196723 |
| HSbrain_627_r | 214.4670798 | 0.148196723 |
| HSbrain_624_l | 94.53967575 | 0.148196723 |
| HS_from_MQbrain_1444_l | 6.459126984 | 0.148196723 |
| HS_from_PT_134_l | 371.7744239 | 0.149149579 |
| HS_139_l | 299.5699263 | 0.149149579 |
| HS_58_l | 238.3731335 | 0.149149579 |
| HS_from_MQbrain_1088_l | 194.3140687 | 0.149149579 |
| HS_from_MQbrain_1773_l | 173.1308074 | 0.149149579 |
| HS_from_MQbrain_1419_r | 158.7663804 | 0.149149579 |
| HSbrain_177_r | 132.197755 | 0.149149579 |
| HS_from_MQbrain_817_l | 7.907551804 | 0.149149579 |
| Hsd_81_r | 3.432967686 | 0.149149579 |
| HS_111_r | 266.2298017 | 0.152177614 |
| HSbrain_717_l | 241.5872727 | 0.152177614 |
| HSbrain_41_l | 239.6624516 | 0.152177614 |
| HSbrain_304_l | 204.8368425 | 0.152177614 |
| HS_from_PT_20_r | 188.3393313 | 0.152177614 |
| Hsd_from_Mmd_509_l | 115.5595143 | 0.152177614 |
| HS_204_r | 14.93137289 | 0.152177614 |
| HSbrain_625_l | 5.435431773 | 0.152177614 |
| HS_from_MQbrain_1433_r | 204.5305106 | 0.155479351 |
| HSbrain_578_r | 203.0612339 | 0.155479351 |
| HS_from_MQbrain_1128_l | 158.837926 | 0.155479351 |
| HS_from_MQbrain_536_r | 150.565992 | 0.155479351 |
| HSbrain_431_l | 329.5258621 | 0.15865589 |
| HSbrain_673_l | 313.971615 | 0.15865589 |
| HS_from_MQbrain_1601_r | 288.3631003 | 0.15865589 |
| HSbrain_652_l | 240.7492888 | 0.15865589 |
| HSbrain_202_r | 317.8857843 | 0.159708794 |
| HS_from_MQbrain_604_l | 289.730194 | 0.159708794 |
| Hsd_from_Mmd_480_r | 278.0451339 | 0.159708794 |
| HS_167_r | 253.7791765 | 0.159708794 |
| HS_from_MQbrain_529_r | 190.4031906 | 0.159708794 |
| HS_from_PT_254_r | 38.90953686 | 0.159708794 |
| HS_from_MQbrain_1882_r | 15.42159484 | 0.159708794 |
| HS_from_MQbrain_1753_r | 15.00695265 | 0.159708794 |
| HS_292_r | 3.304463889 | 0.159708794 |
| HSbrain_644_r | 2.20892511 | 0.159708794 |
| HS_from_MQbrain_1877_r | 232.7961089 | 0.161647438 |
| HSbrain_72_l | 206.0852876 | 0.161647438 |
| HS_from_MQbrain_1839_l | 186.2075177 | 0.161647438 |
| HS_288_l | 171.5705836 | 0.161647438 |
| HSbrain_16_l | 14.42037004 | 0.161647438 |
| HSbrain_220_l | 226.9273128 | 0.16463875 |
| HS_208_r | 211.3635201 | 0.16463875 |
| HS_from_PT_41_l | 209.4264654 | 0.16463875 |
| HS_from_MM_319_l | 202.6221234 | 0.16463875 |
| HS_from_MQbrain_1229_r | 179.8888306 | 0.16463875 |
| Hsd_from_Mmd_223_r | 19.94197059 | 0.16463875 |
| HSbrain_111_l | 19.09214846 | 0.16463875 |
| HS_130_l | 10.32443146 | 0.16463875 |
| HSbrain_168_l | 327.7976202 | 0.167313007 |
| HS_from_MQbrain_1876_r | 294.6889637 | 0.167313007 |
| HSbrain_513_r | 225.3093848 | 0.167313007 |
| HS_from_MQbrain_699_r | 210.3435386 | 0.167313007 |
| HS_from_MQbrain_1372_l | 173.5611463 | 0.167313007 |
| HS_from_MQbrain_2670_r | 88.75898562 | 0.167313007 |
| HS_from_MQbrain_496_r | 276.9628577 | 0.170255758 |
| HS_from_MQbrain_1855_l | 238.2044165 | 0.170255758 |
| HS_114_l | 214.0153294 | 0.170255758 |
| HSbrain_113_r | 100.6056081 | 0.170255758 |
| HSbrain_614_l | 255.7168248 | 0.173467438 |
| HS_29_l | 201.5999875 | 0.173467438 |
| HS_from_MQbrain_57_l | 186.5917929 | 0.173467438 |
| HSbrain_336_l | 103.7156038 | 0.173467438 |
| HSbrain_777_r | 5.13504626 | 0.173467438 |
| HS_from_MQbrain_734_l | 272.5872546 | 0.175200251 |
| HS_from_MQbrain_1777_l | 271.5768067 | 0.175200251 |
| HS_from_MQbrain_1463_l | 269.5851659 | 0.175200251 |
| HSbrain_687_l | 261.7764626 | 0.175200251 |
| HS_from_PT_34_r | 259.0811135 | 0.175200251 |
| HSbrain_643_l | 212.7527559 | 0.175200251 |
| HS_from_MQbrain_1091_l | 169.2750909 | 0.175200251 |
| HS_from_MQbrain_1708_l | 154.1529962 | 0.175200251 |
| Hsd_from_Mmd_167_r | 352.3358142 | 0.177405071 |
| HS_from_MQbrain_1470_r | 320.2896415 | 0.177405071 |
| HSbrain_692_r | 285.9738106 | 0.177405071 |
| Hsd_from_Mmd_42_r | 266.6423152 | 0.177405071 |
| HSbrain_794_l | 265.9452768 | 0.177405071 |
| HS_from_MQbrain_386_r | 231.6815526 | 0.177405071 |
| HSbrain_690_l | 231.2618802 | 0.177405071 |
| HSbrain_347_l | 211.107069 | 0.177405071 |
| HSbrain_1305_l | 145.0316211 | 0.177405071 |
| HS_from_MQbrain_1485_r | 90.24185865 | 0.177405071 |
| HS_from_MQbrain_1442_l | 5.874225415 | 0.177405071 |
| HS_from_MQbrain_550_l | 253.7757692 | 0.180260145 |
| HS_285_r | 233.586665 | 0.180260145 |
| HS_from_MQbrain_1631_r | 227.5696524 | 0.180260145 |
| HSbrain_593_r | 222.0517315 | 0.180260145 |
| HSbrain_263_r | 108.4481826 | 0.180260145 |
| HS_from_MQbrain_991_r | 72.5119085 | 0.180260145 |
| HS_from_MQbrain_1110_r | 6.60657006 | 0.180260145 |
| Hsd_from_Mmd_286_r | 5.6723549 | 0.180260145 |
| HSbrain_778_l | 276.1268612 | 0.184337179 |
| Hsd_from_Mmd_120_r | 256.1765933 | 0.184337179 |
| HSbrain_621_l | 240.1453763 | 0.184337179 |
| HS_243_l | 211.6547669 | 0.184337179 |
| HS_138_l | 198.1359437 | 0.184337179 |
| HS_from_MM_131_l | 3.843700557 | 0.184337179 |
| HSbrain_166_l | 305.2218421 | 0.185815478 |
| HS_from_MQbrain_1394_l | 277.0531964 | 0.185815478 |
| HSbrain_1364_l | 251.9113433 | 0.185815478 |
| HS_142_l | 223.5805612 | 0.185815478 |
| HS_from_MQbrain_272_l | 218.2733075 | 0.185815478 |
| HSbrain_32_l | 216.2330231 | 0.185815478 |
| HS_from_MQbrain_17_r | 210.8657734 | 0.185815478 |
| HS_77_l | 209.1463931 | 0.185815478 |
| HS_303_r | 198.3144581 | 0.185815478 |
| HSbrain_662_r | 47.55963248 | 0.185815478 |
| HS_from_MQbrain_1778_r | 16.56308131 | 0.185815478 |
| Hsd_from_Mmd_48_r | 4.488918042 | 0.185815478 |
| HSbrain_141_r | 277.8002235 | 0.188706417 |
| HS_50_l | 272.9010802 | 0.188706417 |
| HS_from_MQbrain_745_r | 271.8779897 | 0.188706417 |
| HS_from_MQbrain_36_r | 247.5789144 | 0.188706417 |
| HS_from_MQbrain_940_r | 239.4870149 | 0.188706417 |
| HS_from_MQbrain_691_r | 205.3284172 | 0.188706417 |
| HSbrain_570_l | 194.0529436 | 0.188706417 |
| HS_189_l | 117.9592496 | 0.188706417 |
| HSbrain_497_l | 22.40769173 | 0.188706417 |
| Hsd_96_r | 5.165955532 | 0.188706417 |
| HS_241_r | 2.716303039 | 0.188706417 |
| HS_from_MQbrain_397_l | 374.0176792 | 0.190550803 |
| HS_from_PT_238_l | 251.4029293 | 0.190550803 |
| HS_105_l | 249.0808996 | 0.190550803 |
| HS_from_MM_80_l | 221.3749747 | 0.190550803 |
| HS_166_l | 221.2754845 | 0.190550803 |
| HS_from_MQbrain_97_r | 220.2493082 | 0.190550803 |
| HS_from_MQbrain_626_r | 217.3494318 | 0.190550803 |
| HS_from_MQbrain_968_r | 212.4682002 | 0.190550803 |
| Hsd_62_l | 208.0883252 | 0.190550803 |
| HSbrain_809_r | 156.7134677 | 0.190550803 |
| Hsd_from_Mmd_117_r | 7.718924828 | 0.190550803 |
| HS_from_MQbrain_653_l | 4.501537159 | 0.190550803 |
| Hsd_from_Mmd_163_r | 4.482580882 | 0.190550803 |
| HS_from_MQbrain_675_l | 3.821332516 | 0.190550803 |
| HSbrain_259_r | 334.9867442 | 0.194339793 |
| HS_from_MQbrain_1272_l | 282.8549789 | 0.194339793 |
| HS_from_MQbrain_1421_l | 270.6118637 | 0.194339793 |
| Hsd_from_Mmd_298_r | 48.27315482 | 0.194339793 |
| HSbrain_755_l | 2.63850582 | 0.194339793 |
| HS_from_MQbrain_1159_l | 267.9987738 | 0.197457594 |
| HS_from_MQbrain_635_r | 239.4679434 | 0.197457594 |
| HSbrain_479_l | 219.1097463 | 0.197457594 |
| HSbrain_210_l | 218.1425564 | 0.197457594 |
| HS_from_MQbrain_467_l | 209.268142 | 0.197457594 |
| HS_from_MQbrain_1514_r | 8.3514592 | 0.197457594 |
| HS_from_MQbrain_1210_l | 318.0464645 | 0.199721903 |
| HS_from_MQbrain_1357_l | 252.6330054 | 0.199721903 |
| HSbrain_388_r | 213.3920423 | 0.199721903 |
| HSbrain_651_r | 190.7821606 | 0.199721903 |
| HSbrain_482_l | 110.0149117 | 0.199721903 |
| HS_from_MM_63_l | 31.12592861 | 0.199721903 |
| HS_from_MQbrain_1405_l | 28.83096971 | 0.199721903 |
| Hsd_from_Mmd_478_l | 24.52473283 | 0.199721903 |
| HSbrain_22_l | 7.394968873 | 0.199721903 |
| Hsd_41_r | 2.227383669 | 0.199721903 |

¹Cut off Signal/background > 2
²Cut off adj p < 0.2

TABLE 11b
Small RNAs recovered from faeces and differentially expressed in
adenocarcinoma vs adenoma: hairpin arm with lowest expression

| | | carcinoma |
|---|---|---|
| | 2^{nd} arm signal/ | vs adenoma |
| Systematic name | background¹ | (adj p)² |
| HS_from_MQbrain_1100_r | 120.6214414 | 0.199721903 |
| HS_from_MQbrain_1747_l | 16.76994109 | 0.199721903 |
| HS_from_MQbrain_229_l | 4.081069893 | 0.199721903 |
| HS_from_MQbrain_1517_r | 2.83503588 | 0.199721903 |
| HSbrain_289_r | 4.948455623 | 0.197457594 |
| HSbrain_190_r | 4.058257011 | 0.197457594 |
| HS_from_MQbrain_819_r | 5.824652829 | 0.190550803 |
| HS_from_MQbrain_1437_l | 2.717424689 | 0.190550803 |
| Hsd_from_Mmd_471_r | 16.97699294 | 0.188706417 |
| HS_from_PT_126_l | 6.759793869 | 0.188706417 |
| HS_from_MM_256_r | 3.393653412 | 0.185815478 |
| HS_177_l | 39.31124871 | 0.184337179 |
| Hsd_84_r | 14.08450328 | 0.184337179 |
| HSbrain_180_l | 9.145529202 | 0.180260145 |
| HS_from_MQbrain_85_l | 156.4403527 | 0.175200251 |
| HS_from_MQbrain_1752_l | 22.86665735 | 0.175200251 |
| HS_231_l | 2.714286111 | 0.175200251 |
| HSbrain_429_l | 2.118433497 | 0.175200251 |
| HS_from_MM_6_r | 2.160347119 | 0.173467438 |
| HS_from_MQbrain_1376_r | 12.27563489 | 0.167313007 |
| HS_from_MQbrain_42_l | 10.06865266 | 0.167313007 |
| HS_from_MQbrain_1268_l | 5.668043125 | 0.167313007 |
| HS_from_MQbrain_808_l | 18.66016883 | 0.16463875 |
| HS_from_MQbrain_1722_l | 110.8769733 | 0.161647438 |
| HS_from_MQbrain_2441_r | 18.63671222 | 0.161647438 |
| HS_from_MQbrain_635_l | 3.030934579 | 0.161647438 |
| HS_from_MM_124_l | 2.285310959 | 0.161647438 |
| HS_from_PT_91_r | 11.52114628 | 0.159708794 |
| HS_from_MQbrain_1168_l | 20.50912815 | 0.15865589 |
| Hsd_from_Mmd_58_r | 3.243424426 | 0.15865589 |
| Hsd_66_l | 18.13978635 | 0.152177614 |
| HS_from_MQbrain_666_l | 92.89661383 | 0.149149579 |
| HS_from_MQbrain_535_r | 186.6969315 | 0.145370181 |
| HS_from_MQbrain_64_l | 11.00783751 | 0.145370181 |
| HSbrain_381_l | 4.039805518 | 0.145370181 |
| HS_from_MQbrain_770_r | 5.072552769 | 0.14474535 |
| Hsd_from_Mmd_123_r | 3.109340734 | 0.14474535 |
| HS_from_MQbrain_809_l | 18.13661315 | 0.139019426 |
| HSbrain_174_r | 4.177116441 | 0.136637202 |
| HSbrain_776_l | 114.3914887 | 0.130803986 |
| Hsd_from_Mmd_219_r | 42.949196 | 0.130803986 |
| HS_from_MQbrain_800_l | 20.11153179 | 0.129555956 |
| HS_from_MQbrain_140_l | 5.24618389 | 0.129555956 |
| HS_from_MQbrain_814_l | 11.67524525 | 0.124263194 |
| HS_from_MQbrain_1633_r | 10.30485548 | 0.124263194 |
| HS_from_MQbrain_268_r | 3.108624251 | 0.124263194 |
| HS_from_MQbrain_1412_l | 4.664856717 | 0.12300785 |
| HS_from_MM_180_r | 2.63242039 | 0.119559457 |
| HS_from_MM_331_r | 5.344166534 | 0.115928328 |
| HS_from_MQbrain_842_l | 2.595893731 | 0.114595796 |
| HS_from_MQbrain_1339_r | 21.84300167 | 0.11066006 |
| HS_21_r | 216.9668884 | 0.107794024 |
| HS_from_MQbrain_1783_r | 5.660060677 | 0.105993846 |
| HS_from_MQbrain_1116_r | 48.72741718 | 0.104515233 |
| HS_from_MQbrain_1748_r | 19.48179132 | 0.101009439 |
| HS_from_MQbrain_1771_l | 188.7899392 | 0.098301345 |
| HSbrain_553_r | 7.093626312 | 0.098301345 |
| HS_from_MQbrain_1481_l | 32.02173709 | 0.089491072 |
| HS_from_MQbrain_597_r | 4.765274065 | 0.084885584 |
| HS_from_MQbrain_824_l | 2.021767126 | 0.084885584 |
| HS_from_MQbrain_1087_l | 8.47223421 | 0.083833911 |
| HSbrain_319_l | 27.65786021 | 0.07965977 |
| HS_from_MQbrain_1462_l | 135.9829616 | 0.07715065 |
| HSbrain_253_l | 11.33164065 | 0.07715065 |
| Hsd_105_l | 5.399875994 | 0.07715065 |
| HS_from_MQbrain_881_r | 3.552631725 | 0.07715065 |
| Hsd_from_Mmd_410_r | 2.908576361 | 0.07641095 |
| Hsd_from_Mmd_464_l | 2.017405172 | 0.07641095 |
| HS_from_MQbrain_266_r | 7.219897398 | 0.075028175 |
| HS_from_MQbrain_435_r | 2.200043662 | 0.072654718 |
| HSbrain_93_r | 4.274468514 | 0.072275086 |
| HS_from_MQbrain_932_l | 2.123754543 | 0.068270026 |
| Hsd_from_Mmd_371_r | 131.2157622 | 0.058218978 |
| HS_from_MQbrain_1070_r | 19.32265622 | 0.058218978 |
| HS_from_MQbrain_1628_r | 3.67815753 | 0.051819249 |
| HS_from_MQbrain_1565_l | 11.415781 | 0.050135128 |
| HSbrain_703_r | 11.82131557 | 0.047341086 |
| HS_from_MM_251_r | 168.6680712 | 0.039250091 |
| Hsd_85_r | 3.438673873 | 0.034662803 |
| HS_from_MQbrain_1500_l | 15.2774537 | 0.023919533 |
| HS_from_MQbrain_941_r | 2.300164622 | 0.016904198 |

¹Cut off Signal/background > 2
²Cut off adj p < 0.2

### Presence of Classifying Small RNAs in Faeces

TABLE 12a
Classification of control vs adenoma
The following 21 out of 55 classifying small RNAs can be detected in blood

| | | |
|---|---|---|
| HSbrain_377_r | Hsd_from_Mmd_452_l | HSbrain_224_r |
| HS_from_MM_118_r | Hsd_from_Mmd_117_r | HS_169_l |
| HS_from_MM_169_r | HS_from_MQbrain_79_r | HS_from_MQbrain_825_r |
| HS_from_MQbrain_594_r | HSbrain_106_r | HSbrain_1_l |
| HS_from_PT_36_l | HS_262_l | HS_from_MQbrain_1823_r |
| Hsd_from_Mmd_298_r | HS_from_MQbrain_1311_r | HS_from_MQbrain_194_l |
| Hsd_from_Mmd_485_r | HS_from_MQbrain_1462_l | |
| HSbrain_1406_l | HS_from_MQbrain_1183_l | |

TABLE 12b
Classification of control vs adenocarcinoma
The following 16 out of 79 classifying small RNAs can be
detected in blood

HS_from_MQbrain_1320_r
HS_from_MQbrain_594_r
HS_from_MQbrain_855_r
HSbrain_426_r
HS_from_MQbrain_340_l
HS_from_MM_353_l
HS_from_MM_210_l
HSbrain_704_l
HS_from_MQbrain_485_l
Hsd_from_Mmd_328_l
HS_from_MQbrain_1432_l
HS_from_MM_178_l
HSbrain_499_l
HS_from_MQbrain_827_l
HS_from_MM_47_r

TABLE 12c
Classification of adenoma vs adenocarcinoma
The following 4 out of 26 classifying small RNAs can be detected in blood

HS_from_MQbrain_1082_l
HS_from_PT_36_l
HS_from_MQbrain_824_l
HS_from_MQbrain_825_r

TABLE 13
Recovered small RNAs in blood

| | 1st arm |
|---|---|
| Systematic name | signal/backgr¹ |
| HSbrain_685_l | 4143.44 |
| Hsd_12_l | 3822.46 |
| HS_from_MQbrain_1364_l | 3810.98 |
| HS_from_MQbrain_1412_r | 3753.01 |
| HS_from_MM_111_r | 3707.66 |
| Hsd_34_l | 3535.17 |
| HSbrain_550_r | 3519.10 |
| HSbrain_188_r | 3508.56 |
| HS_from_MQbrain_1804_l | 3483.45 |
| HSbrain_221_l | 3453.74 |
| HS_199_r | 3400.22 |
| Hsd_from_Mmd_535_l | 3397.88 |
| HS_from_MQbrain_1426_l | 3397.58 |
| HS_from_MQbrain_1777_l | 3386.15 |
| HS_72_r | 3347.62 |
| HS_from_MQbrain_1683_l | 3345.13 |
| HSbrain_10_l | 3325.13 |
| HSbrain_328_l | 3271.08 |
| HS_from_MQbrain_533_l | 3263.08 |
| HS_from_MQbrain_1741_l | 3259.71 |
| HS_from_MQbrain_1863_l | 3259.56 |
| HSbrain_160_l | 3253.62 |
| HSbrain_172_r | 3241.21 |
| Hsd_from_Mmd_185_l | 3235.16 |
| HS_from_MQbrain_655_r | 3221.03 |
| HS_from_MQbrain_1219_l | 3215.41 |
| HSbrain_664_r | 3210.45 |
| HS_from_MQbrain_1306_r | 3210.23 |
| HSbrain_731_l | 3209.06 |
| HSbrain_769_r | 3201.06 |
| HS_from_MQbrain_487_r | 3196.14 |
| HS_from_MQbrain_108_l | 3190.76 |
| HS_from_MQbrain_1652_r | 3171.46 |
| HSbrain_55_r | 3168.27 |
| HS_from_MQbrain_1727_r | 3152.31 |
| HSbrain_679_l | 3143.27 |
| HS_from_MQbrain_115_r | 3142.08 |
| Hsd_from_Mmd_448_r | 3129.79 |
| HSbrain_299_l | 3118.98 |
| HS_from_MQbrain_1709_r | 3100.66 |
| HSbrain_611_l | 3099.01 |
| HS_48_l | 3088.27 |
| HS_from_MQbrain_330_r | 3085.23 |
| HS_from_MQbrain_265_r | 3084.01 |
| HS_from_MQbrain_111_r | 3079.60 |
| HS_173_r | 3074.67 |
| HS_from_MQbrain_229_r | 3071.27 |
| HS_78_l | 3066.99 |
| HSbrain_191_l | 3066.36 |
| HS_32_l | 3064.10 |
| HSbrain_244_r | 3058.16 |
| Hsd_from_Mmd_472_r | 3055.30 |
| HS_from_MQbrain_428_r | 3049.36 |
| HS_from_MQbrain_459_l | 3044.63 |
| HSbrain_1377_r | 3044.23 |
| HS_from_MQbrain_1632_l | 3014.17 |
| Hsd_from_Mmd_487_l | 3011.40 |
| HSbrain_676_l | 3009.99 |
| HS_19_l | 3008.92 |
| HS_from_PT_64_r | 3003.40 |
| HS_284_l | 2997.74 |
| HSbrain_306_r | 2986.89 |
| HS_from_MM_103_l | 2974.49 |
| HS_from_MQbrain_734_l | 2968.08 |
| HS_from_MQbrain_1575_r | 2955.83 |
| HS_from_MQbrain_1848_l | 2953.59 |
| HS_from_MQbrain_1241_l | 2950.15 |
| HS_from_MQbrain_688_l | 2949.66 |
| HS_from_PT_305_l | 2949.38 |
| HSbrain_279_r | 2940.84 |
| HSbrain_210_l | 2938.20 |
| HS_from_MQbrain_1380_r | 2936.43 |
| HS_from_MQbrain_1599_l | 2929.74 |
| HS_from_MQbrain_1084_r | 2928.75 |
| HS_from_MQbrain_1803_l | 2927.91 |
| HS_from_MQbrain_311_l | 2926.98 |
| HSbrain_152_l | 2922.92 |
| HSbrain_1407_l | 2916.25 |
| HSbrain_334_l | 2905.21 |
| HSbrain_114_l | 2900.41 |
| HS_from_MQbrain_1697_r | 2900.13 |
| HS_from_MQbrain_1134_l | 2892.26 |
| HSbrain_425_r | 2881.36 |
| Hsd_from_Mmd_405_r | 2881.01 |
| HS_from_MQbrain_1399_l | 2880.82 |
| HS_from_PT_134_l | 2880.24 |
| HS_163_l | 2877.66 |
| Hsd_21_l | 2876.65 |
| HS_from_MQbrain_715_l | 2873.52 |
| HSbrain_783_l | 2871.64 |
| HS_from_MQbrain_1509_r | 2861.75 |
| HS_from_MQbrain_1686_l | 2850.33 |
| HSbrain_326_l | 2841.30 |
| HSbrain_404_r | 2841.00 |
| HS_from_PT_18_l | 2829.14 |
| HSbrain_440_l | 2827.75 |
| HSbrain_141_r | 2820.17 |
| HS_from_MQbrain_810_l | 2819.61 |
| HS_from_MM_5_r | 2818.77 |
| HSbrain_283_l | 2809.33 |
| HS_from_MQbrain_1236_l | 2808.42 |
| HS_135_r | 2806.71 |
| HS_from_MQbrain_324_r | 2803.91 |
| HS_from_MQbrain_184_r | 2802.82 |
| HSbrain_1298_l | 2802.59 |
| HS_from_PT_226_r | 2799.23 |
| HSbrain_313_r | 2798.16 |
| HS_198_l | 2797.81 |
| Hsd_50_l | 2796.99 |
| HS_from_MQbrain_1365_r | 2796.98 |
| HS_from_MQbrain_863_r | 2785.40 |
| HS_from_MQbrain_610_r | 2783.73 |
| HS_from_MQbrain_877_l | 2776.99 |
| HS_from_MQbrain_33_l | 2774.13 |
| HS_from_PT_102_l | 2772.91 |
| HS_75_l | 2768.60 |
| HS_from_MQbrain_1581_l | 2763.55 |
| HS_from_MQbrain_387_l | 2762.99 |
| HSbrain_250_l | 2758.18 |
| HSbrain_1236_r | 2749.95 |
| HSbrain_45_r | 2746.16 |
| HS_from_MQbrain_301_r | 2746.14 |
| HSbrain_367_r | 2743.94 |
| HS_from_MQbrain_129_l | 2741.16 |
| HS_from_PT_92_l | 2739.09 |
| HSbrain_240_l | 2736.22 |
| HS_from_MQbrain_345_r | 2731.68 |
| HS_from_MQbrain_1523_r | 2731.64 |
| HSbrain_365_r | 2731.35 |
| HS_from_MQbrain_996_l | 2730.34 |
| HS_9_l | 2720.49 |
| HSbrain_688_l | 2718.05 |
| HS_197_r | 2713.25 |
| HSbrain_745_r | 2713.11 |
| HS_from_MQbrain_1331_r | 2711.38 |
| HS_from_MQbrain_1864_r | 2711.06 |
| HS_from_MQbrain_1631_r | 2710.28 |
| HS_113_r | 2700.28 |
| HSbrain_192_l | 2698.44 |
| HS_133_l | 2696.57 |
| HS_from_MQbrain_765_l | 2695.54 |
| HSbrain_109_l | 2695.37 |
| HS_from_MQbrain_935_r | 2688.96 |
| HS_from_MQbrain_900_l | 2685.43 |
| HS_from_MM_346_l | 2685.36 |
| HS_171_l | 2683.04 |
| HS_from_MQbrain_1812_l | 2682.09 |
| HSbrain_680_l | 2680.84 |
| HSbrain_761_r | 2676.91 |
| HS_from_MM_45_l | 2671.67 |
| Hsd_46_r | 2671.21 |
| HS_from_PT_158_l | 2670.87 |
| HS_from_MQbrain_142_l | 2670.59 |
| HS_from_PT_115_l | 2669.44 |
| HS_from_MQbrain_48_l | 2663.41 |
| HS_from_MQbrain_1824_l | 2661.19 |
| HS_from_MQbrain_1827_l | 2658.72 |
| HS_from_MQbrain_1750_r | 2658.65 |
| HSbrain_797_l | 2657.20 |
| HS_from_PT_204_r | 2648.95 |
| HS_from_MQbrain_1006_r | 2648.14 |
| HS_111_r | 2642.45 |
| HSbrain_668_l | 2641.86 |
| HSbrain_713_l | 2635.96 |
| HS_from_MQbrain_70_l | 2632.56 |
| HS_from_MQbrain_102_r | 2631.46 |
| HS_from_MQbrain_496_r | 2625.70 |
| HS_from_MQbrain_508_l | 2623.33 |
| HS_from_PT_34_r | 2623.29 |
| HS_from_MQbrain_1838_l | 2619.34 |
| HS_from_PT_170_l | 2619.23 |
| HS_from_MQbrain_1548_l | 2618.14 |
| HS_from_PT_44_l | 2616.23 |
| HSbrain_167_l | 2615.58 |
| Hsd_from_Mmd_167_r | 2610.41 |
| HS_from_MQbrain_498_r | 2610.14 |
| HS_from_PT_89_l | 2608.60 |
| HSbrain_400_r | 2608.12 |
| HS_from_MQbrain_1062_r | 2607.05 |
| HS_55_l | 2605.59 |
| HS_from_MQbrain_550_l | 2602.74 |
| HS_from_MQbrain_1021_l | 2602.48 |
| HS_from_MQbrain_520_r | 2601.96 |
| Hsd_36_r | 2600.28 |
| HS_176_r | 2594.07 |
| HSbrain_493_l | 2590.11 |
| HS_from_MQbrain_1876_r | 2589.02 |
| HS_from_MQbrain_336_l | 2585.50 |
| HS_from_MQbrain_1450_l | 2585.11 |
| HS_from_MQbrain_737_l | 2582.59 |
| HSbrain_56_l | 2577.80 |
| HS_from_MQbrain_566_r | 2574.83 |
| HS_263_r | 2573.65 |
| HS_from_MQbrain_1616_l | 2568.30 |
| Hsd_from_Mmd_78_l | 2567.31 |
| HS_from_MQbrain_544_r | 2566.93 |
| HS_190_l | 2565.53 |
| HS_from_MQbrain_11_r | 2564.86 |
| HS_from_MQbrain_429_r | 2564.38 |
| HS_from_MQbrain_698_l | 2562.25 |
| HSbrain_171_l | 2560.96 |
| HS_from_MQbrain_1798_r | 2560.70 |
| HS_from_PT_26_l | 2560.41 |
| HSbrain_1420_l | 2559.24 |
| HS_from_MQbrain_1421_l | 2558.01 |
| HS_from_MQbrain_379_l | 2556.32 |
| HS_from_MQbrain_1091_l | 2553.31 |
| HS_from_MQbrain_198_l | 2550.99 |
| Hsd_72_r | 2546.93 |
| HSbrain_139_l | 2546.02 |
| HSbrain_31_l | 2543.80 |
| HSbrain_614_l | 2539.17 |
| HSbrain_216_r | 2537.24 |
| HS_from_MM_174_r | 2535.71 |
| HS_from_MQbrain_1780_l | 2528.11 |
| HSbrain_659_r | 2528.00 |
| HSbrain_518_l | 2526.36 |
| HS_from_MQbrain_1641_l | 2520.73 |
| HS_from_MM_318_l | 2515.65 |
| HSbrain_457_l | 2514.23 |
| Hsd_39_l | 2514.08 |
| HS_from_MQbrain_1867_l | 2513.58 |
| HS_from_MM_355_l | 2507.99 |
| HS_from_MQbrain_1447_r | 2506.39 |
| HSbrain_746_r | 2502.99 |
| HS_from_MQbrain_1053_r | 2502.67 |
| HS_from_MM_339_r | 2501.25 |
| HS_from_MM_347_l | 2499.97 |
| HS_from_MM_200_r | 2499.90 |
| HS_from_MQbrain_1891_l | 2495.79 |
| HSbrain_307_r | 2494.19 |
| HS_from_MQbrain_1075_l | 2494.05 |
| HS_from_MQbrain_1160_l | 2492.15 |
| HSbrain_728_r | 2490.79 |
| HS_from_PT_112_l | 2489.56 |
| HS_from_MQbrain_491_l | 2488.59 |
| HS_from_MQbrain_1681_l | 2488.34 |
| HS_from_MQbrain_1085_r | 2488.16 |
| HSbrain_166_l | 2487.26 |
| HSbrain_800_l | 2486.16 |
| HS_from_PT_178_l | 2486.13 |
| HS_from_MQbrain_1663_l | 2485.79 |
| HS_from_MQbrain_1873_r | 2484.30 |
| HS_from_MQbrain_1261_r | 2483.06 |
| HSbrain_472_r | 2477.23 |
| HS_from_MQbrain_626_r | 2474.16 |
| HS_from_MQbrain_181_r | 2473.00 |
| HSbrain_759_r | 2472.80 |
| HS_from_MQbrain_1833_r | 2472.66 |
| HSbrain_802_l | 2470.63 |
| HS_from_MQbrain_1635_r | 2468.96 |
| Hsd_from_Mmd_291_r | 2468.16 |
| HS_105_l | 2468.12 |
| HSbrain_669_l | 2467.56 |
| HSbrain_323_l | 2463.41 |
| HSbrain_273_r | 2461.78 |
| HS_from_MQbrain_219_l | 2460.07 |
| HSbrain_1416_l | 2456.16 |
| HS_from_MQbrain_264_r | 2455.84 |
| HS_from_MQbrain_1574_l | 2453.20 |
| Hsd_from_Mmd_237_l | 2452.36 |
| HSbrain_123_l | 2450.67 |
| HSbrain_487_r | 2447.17 |
| HSbrain_786_r | 2443.42 |
| HS_195_r | 2442.99 |
| HSbrain_371_l | 2442.94 |
| HS_from_MQbrain_1711_r | 2442.60 |
| HS_240_r | 2437.75 |
| HS_157_l | 2437.27 |
| HS_from_MQbrain_893_l | 2433.34 |
| HSbrain_673_l | 2432.23 |
| HS_from_MQbrain_660_r | 2430.71 |
| HS_from_MQbrain_1102_l | 2428.54 |
| Hsd_from_Mmd_212_l | 2425.64 |
| HS_155_l | 2415.37 |
| HS_from_MQbrain_1353_r | 2413.71 |
| HSbrain_588_l | 2412.89 |
| HS_from_MQbrain_901_l | 2410.60 |
| HS_from_MQbrain_927_l | 2409.98 |
| HS_from_MQbrain_753_l | 2409.46 |
| HS_from_MQbrain_745_r | 2408.79 |
| HS_154_l | 2406.35 |
| HSbrain_708_l | 2404.97 |
| HS_from_MQbrain_42_r | 2403.66 |
| HSbrain_717_l | 2399.92 |
| HSbrain_125_r | 2389.54 |
| HS_98_r | 2382.13 |
| HS_from_MQbrain_1222_r | 2381.15 |
| HS_65_l | 2379.67 |
| HS_from_MQbrain_1889_l | 2376.83 |
| HS_from_MQbrain_1504_r | 2372.55 |
| HSbrain_8_r | 2366.39 |
| HS_from_MQbrain_89_r | 2363.91 |
| HSbrain_548_l | 2363.14 |
| HS_from_MQbrain_1355_r | 2362.21 |
| HSbrain_361_l | 2360.92 |
| HSbrain_48_l | 2360.91 |
| HS_from_MQbrain_2679_r | 2360.09 |
| HS_from_MQbrain_1198_l | 2359.66 |
| HS_3_l | 2356.16 |
| HS_from_MQbrain_1073_r | 2353.25 |
| HS_from_MM_340_l | 2350.89 |
| Hsd_from_Mmd_433_r | 2349.66 |
| HSbrain_607_l | 2346.48 |
| HSbrain_621_l | 2344.81 |
| HS_from_MQbrain_1874_r | 2343.16 |
| HS_from_MQbrain_638_r | 2341.76 |
| HSbrain_315_l | 2340.73 |
| HS_from_MQbrain_1767_l | 2338.11 |
| HS_from_MQbrain_1416_l | 2337.11 |
| HSbrain_132_r | 2337.03 |
| HS_201_l | 2336.02 |
| HSbrain_391_l | 2333.94 |
| HS_from_MQbrain_642_r | 2329.37 |
| HS_from_MQbrain_1074_l | 2328.54 |
| HS_from_MQbrain_1415_l | 2327.82 |
| HS_167_r | 2325.07 |
| HS_from_PT_197_l | 2324.67 |
| HS_from_MQbrain_1832_l | 2323.53 |
| HS_from_MQbrain_1458_r | 2322.87 |
| HS_from_MQbrain_1083_l | 2322.20 |
| HS_from_MQbrain_766_l | 2320.70 |
| HS_from_MQbrain_1311_l | 2319.68 |
| HS_from_MQbrain_1470_r | 2318.58 |
| HSbrain_692_r | 2311.79 |
| HS_from_MQbrain_493_r | 2308.93 |
| HS_from_MQbrain_448_l | 2308.91 |
| HS_from_MQbrain_87_l | 2306.16 |
| Hsd_from_Mmd_261_l | 2305.13 |
| HS_101_l | 2303.10 |
| HSbrain_305_r | 2302.79 |
| HS_from_MQbrain_885_r | 2301.62 |
| HS_from_MQbrain_1119_l | 2299.12 |
| HS_from_MQbrain_303_l | 2295.05 |
| HSbrain_460_l | 2292.26 |
| HS_89_l | 2291.35 |
| HSbrain_485_r | 2284.67 |
| HSbrain_677_l | 2284.34 |
| HSbrain_410_r | 2283.80 |
| HSbrain_402_l | 2283.48 |
| HS_from_MQbrain_1761_l | 2283.06 |
| HSbrain_810_l | 2281.81 |
| HS_from_MQbrain_712_l | 2277.72 |
| HS_from_MQbrain_1862_r | 2275.81 |
| HS_from_MM_229_l | 2273.58 |
| HS_from_MQbrain_1633_l | 2271.80 |
| HSbrain_508_r | 2266.14 |
| HS_from_PT_71_l | 2264.20 |
| HS_from_MQbrain_947_r | 2263.52 |
| HS_from_PT_295_l | 2261.78 |
| HS_from_MQbrain_1228_r | 2260.21 |
| HS_219_l | 2255.05 |
| HS_from_MQbrain_697_r | 2254.48 |
| HS_from_MQbrain_1596_r | 2252.81 |
| HSbrain_431_l | 2250.77 |
| HS_102_l | 2248.72 |
| HSbrain_33_l | 2248.37 |
| HS_from_MQbrain_1018_l | 2246.41 |
| HS_from_MQbrain_1839_l | 2242.94 |
| HS_from_MQbrain_397_l | 2241.37 |
| HS_from_MQbrain_59_r | 2237.83 |
| HSbrain_744_r | 2235.39 |
| Hsd_49_r | 2232.71 |
| HSbrain_651_r | 2230.86 |
| HSbrain_143_l | 2228.78 |
| HS_from_MQbrain_672_l | 2227.70 |
| HS_188_r | 2226.82 |
| Hsd_103_r | 2224.79 |
| Hsd_from_Mmd_100_l | 2223.12 |
| HS_from_MQbrain_76_l | 2222.92 |
| HS_from_MQbrain_342_r | 2219.93 |
| HS_from_MQbrain_1834_r | 2219.56 |
| HSbrain_182_r | 2216.91 |
| HSbrain_53_l | 2212.76 |
| HS_from_MM_240_r | 2211.37 |
| HS_from_MQbrain_234_r | 2211.14 |
| HSbrain_439_r | 2209.81 |
| HS_from_MQbrain_1677_l | 2208.26 |
| HS_from_MQbrain_325_l | 2207.15 |
| HS_from_MQbrain_1349_l | 2206.20 |
| HS_from_MQbrain_1814_r | 2204.91 |
| HSbrain_30_l | 2204.53 |
| Hsd_from_Mmd_33_r | 2202.80 |
| Hsd_from_Mmd_215_l | 2202.01 |
| HS_from_MQbrain_1755_l | 2194.99 |
| HS_166_l | 2194.77 |
| HS_64_l | 2193.01 |
| HS_from_MQbrain_1245_r | 2192.77 |
| HS_218_r | 2191.38 |
| HS_from_MM_102_l | 2187.40 |
| HS_from_MQbrain_1237_r | 2185.86 |
| HSbrain_808_l | 2183.22 |
| HSbrain_551_l | 2181.18 |
| HS_from_MQbrain_1553_r | 2180.45 |
| HSbrain_219_r | 2178.54 |
| HS_7_r | 2172.33 |
| HSbrain_284_l | 2165.84 |
| Hsd_66_r | 2160.24 |
| HSbrain_684_l | 2156.24 |
| HS_168_r | 2155.79 |
| HS_from_MQbrain_39_r | 2155.39 |
| HS_from_MQbrain_747_l | 2154.81 |
| HS_from_MQbrain_1689_l | 2153.22 |
| HSbrain_138_l | 2152.79 |
| HSbrain_467_l | 2150.69 |
| HS_274_l | 2148.17 |
| HS_200_l | 2145.99 |
| HS_from_MQbrain_686_r | 2145.46 |
| HS_from_MQbrain_1644_l | 2143.56 |
| HSbrain_266_l | 2143.43 |
| HS_from_MQbrain_999_l | 2142.82 |
| HS_from_MQbrain_1758_r | 2139.74 |
| HS_265_r | 2139.16 |
| HS_from_MQbrain_121_r | 2133.52 |
| HS_from_MQbrain_532_r | 2133.07 |
| HSbrain_318_l | 2130.43 |
| HSbrain_67_l | 2129.48 |
| HS_25_r | 2127.91 |
| HS_from_PT_60_l | 2122.55 |
| HS_from_MQbrain_1224_l | 2122.03 |
| HSbrain_390_r | 2120.00 |
| HSbrain_57_r | 2117.42 |
| HS_from_MQbrain_604_l | 2116.19 |
| HS_from_MQbrain_1455_l | 2115.67 |
| HS_from_MQbrain_1413_l | 2113.32 |
| HS_from_MQbrain_1859_l | 2109.80 |
| HS_from_MQbrain_1446_r | 2109.42 |
| HS_from_MQbrain_941_l | 2108.53 |
| HS_from_MQbrain_914_l | 2107.69 |
| HS_from_MQbrain_163_r | 2105.55 |
| HS_from_MM_80_l | 2105.14 |
| HSbrain_257_l | 2103.60 |
| HS_139_l | 2103.33 |
| HSbrain_463_l | 2102.11 |
| HS_from_MM_324_l | 2099.03 |
| HS_from_MQbrain_852_r | 2095.49 |
| HS_from_MQbrain_1480_r | 2091.30 |
| HS_119_l | 2088.91 |
| Hsd_from_Mmd_299_l | 2087.88 |
| HS_from_MQbrain_218_l | 2087.37 |
| HSbrain_511_r | 2086.28 |
| HS_from_PT_91_l | 2085.31 |
| HS_from_MQbrain_3_r | 2085.03 |
| HS_from_MQbrain_1273_l | 2084.93 |
| HSbrain_6_r | 2079.02 |
| HSbrain_932_r | 2076.26 |
| HS_227_r | 2074.64 |
| HS_from_MQbrain_1204_r | 2072.07 |
| HS_from_MQbrain_1557_r | 2067.16 |
| HS_from_MQbrain_1221_r | 2067.09 |
| HS_from_MQbrain_1199_l | 2060.70 |
| HS_38_l | 2059.93 |
| HS_from_MQbrain_1645_r | 2055.74 |
| HS_from_PT_238_l | 2054.08 |
| HS_from_MQbrain_1159_l | 2053.58 |
| HS_from_MM_115_l | 2053.10 |
| HS_from_MQbrain_40_r | 2048.49 |
| HS_from_MQbrain_644_l | 2042.52 |
| Hsd_90_l | 2037.27 |
| HS_from_MQbrain_716_r | 2025.85 |
| HS_185_l | 2022.92 |
| HS_from_MQbrain_699_r | 2022.54 |
| HSbrain_510_l | 2019.56 |
| HS_from_MQbrain_114_r | 2018.35 |
| HS_from_MM_215_l | 2011.69 |
| HS_148_r | 2011.62 |
| HS_from_PT_185_r | 2009.72 |
| HS_from_MQbrain_1387_l | 2005.33 |
| HS_from_MQbrain_1437_r | 1996.46 |
| HSbrain_72_l | 1994.62 |
| HS_from_MM_208_r | 1993.74 |
| HS_from_MQbrain_1392_r | 1984.76 |
| HSbrain_335_l | 1981.17 |
| HS_from_MQbrain_1007_l | 1977.43 |
| HS_from_MQbrain_1813_r | 1976.78 |
| HS_from_MQbrain_1389_l | 1970.17 |
| HS_from_MM_315_l | 1969.37 |
| HS_from_MQbrain_1357_l | 1967.07 |
| HSbrain_66_r | 1965.12 |
| HSbrain_450_l | 1962.39 |
| HS_from_MM_83_r | 1962.02 |
| HS_170_r | 1953.80 |
| HSbrain_569_l | 1952.86 |
| HS_from_MQbrain_531_r | 1951.46 |
| HS_from_MM_351_r | 1946.67 |
| HS_from_MQbrain_442_r | 1946.64 |
| HS_39_r | 1944.78 |
| HSbrain_703_l | 1943.04 |
| HSbrain_32_l | 1943.03 |
| HS_from_MQbrain_1720_l | 1942.38 |
| HS_from_MQbrain_1696_r | 1942.18 |
| HS_from_MQbrain_553_l | 1940.73 |
| HSbrain_79_l | 1940.49 |
| HS_from_MQbrain_974_l | 1938.97 |
| HSbrain_1247_r | 1936.39 |
| HSbrain_643_l | 1935.82 |
| HSbrain_740_r | 1933.01 |
| HSbrain_396_l | 1932.96 |
| HS_from_MQbrain_436_l | 1932.21 |
| HS_from_MQbrain_576_l | 1930.76 |
| HSbrain_146_r | 1929.15 |
| HSbrain_145_l | 1928.15 |
| HS_from_MQbrain_449_l | 1928.06 |
| HS_from_MQbrain_203_l | 1927.65 |
| Hsd_from_Mmd_510_l | 1923.87 |
| HS_142_l | 1918.30 |
| HS_from_MQbrain_90_r | 1911.93 |
| HS_from_MQbrain_501_l | 1907.55 |
| HS_from_MQbrain_724_l | 1907.01 |
| Hsd_from_Mmd_120_r | 1906.46 |
| HS_from_MQbrain_106_l | 1904.59 |
| Hsd_from_Mmd_198_r | 1903.40 |
| HS_from_MQbrain_392_l | 1903.25 |
| HS_from_MQbrain_636_l | 1902.86 |
| HS_from_MQbrain_257_l | 1901.47 |
| HS_from_MQbrain_1748_l | 1901.30 |
| HS_from_MQbrain_22_r | 1898.63 |
| HSbrain_536_r | 1895.91 |
| HS_from_MQbrain_1367_r | 1893.98 |
| HSbrain_456_l | 1892.99 |
| HS_from_MQbrain_1860_l | 1892.59 |
| HS_from_MQbrain_1313_l | 1890.36 |
| HS_from_MQbrain_539_r | 1889.50 |
| HS_from_MM_304_r | 1888.94 |
| HS_from_MM_234_r | 1885.97 |
| HS_from_MQbrain_546_l | 1884.20 |
| HS_from_MQbrain_93_r | 1882.36 |
| HS_from_PT_262_r | 1880.48 |
| Hsd_from_Mmd_517_l | 1878.43 |
| HS_from_MQbrain_1699_l | 1876.13 |
| HS_from_MQbrain_82_r | 1874.76 |
| HSbrain_44_l | 1864.71 |
| HS_from_MM_295_r | 1858.45 |
| HSbrain_43_l | 1858.02 |
| HSbrain_347_l | 1854.13 |
| HSbrain_696_l | 1853.95 |
| HS_from_MQbrain_210_r | 1848.78 |
| HS_from_MQbrain_925_l | 1846.30 |
| HS_from_MQbrain_1500_r | 1837.75 |
| HS_243_l | 1837.67 |
| HS_259_r | 1836.24 |
| HS_from_MQbrain_1630_l | 1834.56 |
| HS_from_MM_239_l | 1831.11 |
| HSbrain_18_l | 1824.80 |
| HSbrain_892_r | 1822.00 |
| HSbrain_718_l | 1820.31 |
| HSbrain_591_l | 1813.41 |
| HS_from_MQbrain_1360_l | 1813.24 |
| HS_from_MQbrain_1587_r | 1809.89 |
| HS_from_PT_258_l | 1802.88 |
| HS_305_l | 1801.30 |
| HS_from_PT_237_r | 1800.67 |
| HS_122_l | 1800.58 |
| HS_182_l | 1799.59 |
| HS_224_r | 1798.50 |
| HS_from_MQbrain_536_r | 1796.18 |
| HSbrain_549_l | 1796.02 |
| HS_from_MQbrain_1211_l | 1795.91 |
| HSbrain_612_l | 1795.55 |
| HS_from_MQbrain_162_r | 1795.50 |
| HSbrain_1425_l | 1793.99 |
| HS_221_r | 1793.66 |
| HS_129_l | 1787.42 |
| HS_from_MQbrain_1843_r | 1784.80 |
| HS_236_r | 1784.60 |
| HS_50_l | 1776.71 |
| HS_from_MQbrain_807_r | 1776.46 |
| HS_from_MQbrain_1202_l | 1775.29 |
| HS_from_MQbrain_534_r | 1775.11 |
| HS_from_PT_235_r | 1771.52 |
| HS_from_MQbrain_1272_l | 1771.43 |
| HS_194_l | 1770.83 |
| HSbrain_350_l | 1763.12 |
| HS_108_r | 1762.37 |
| HSbrain_809_r | 1761.33 |
| HS_from_MQbrain_341_l | 1757.42 |
| HSbrain_629_l | 1754.85 |
| Hsd_from_Mmd_56_r | 1754.35 |
| HS_from_MQbrain_180_r | 1754.02 |
| HS_from_MQbrain_932_r | 1752.64 |
| HS_from_MQbrain_1756_l | 1749.72 |
| HS_from_MQbrain_297_l | 1746.66 |
| HS_from_MQbrain_332_r | 1740.00 |
| HS_from_MQbrain_1759_r | 1733.64 |
| HSbrain_1413_r | 1730.10 |
| HS_from_MQbrain_631_l | 1729.43 |
| HS_from_MQbrain_36_r | 1728.75 |
| HS_from_MQbrain_99_r | 1728.73 |
| Hsd_from_Mmd_295_r | 1727.90 |
| HS_from_PT_161_r | 1724.84 |
| HS_from_MQbrain_968_r | 1722.97 |
| HSbrain_690_l | 1719.12 |
| HS_from_MQbrain_1844_r | 1717.31 |
| HSbrain_426_r | 1715.96 |
| HS_from_MQbrain_1846_r | 1712.53 |
| HS_from_MQbrain_1785_r | 1710.11 |
| HSbrain_231_l | 1709.15 |
| HS_from_MQbrain_267_l | 1707.93 |
| HS_234_r | 1702.59 |
| HSbrain_227_r | 1702.05 |
| HSbrain_656_r | 1694.07 |
| HS_from_MQbrain_543_l | 1692.71 |
| HS_from_PT_210_r | 1685.32 |
| HSbrain_701_l | 1684.34 |
| HSbrain_101_l | 1684.28 |
| HS_226_r | 1684.20 |
| HS_from_MQbrain_1001_r | 1683.09 |
| HSbrain_628_r | 1681.23 |
| HS_from_MQbrain_452_r | 1678.80 |
| HS_from_MQbrain_529_r | 1669.81 |
| HSbrain_738_r | 1668.40 |
| HS_from_MQbrain_1771_r | 1664.95 |
| HS_288_l | 1661.49 |
| HS_from_MQbrain_824_r | 1660.01 |
| HS_from_MQbrain_561_l | 1659.79 |
| HS_from_PT_214_r | 1659.73 |
| HS_from_MQbrain_770_l | 1656.31 |
| HS_from_MQbrain_1419_r | 1654.34 |
| HS_from_MQbrain_850_l | 1651.86 |
| HS_from_PT_148_l | 1647.57 |
| HS_from_MQbrain_158_r | 1636.63 |
| HS_from_MQbrain_1345_l | 1629.46 |
| HS_from_MM_266_r | 1629.00 |
| HSbrain_821_r | 1626.82 |
| HS_from_PT_286_l | 1623.09 |
| HS_from_MQbrain_375_r | 1621.72 |
| HS_from_MQbrain_281_l | 1617.63 |
| HS_from_MQbrain_146_r | 1605.75 |
| HSbrain_309_r | 1605.65 |
| HS_from_MQbrain_1164_r | 1604.05 |
| HSbrain_259_r | 1597.52 |
| HS_from_MQbrain_635_r | 1594.99 |
| HSbrain_64_r | 1592.09 |
| HS_from_MQbrain_216_l | 1590.82 |
| HS_from_MQbrain_540_l | 1590.52 |
| HSbrain_70_r | 1587.77 |
| HS_from_MQbrain_1890_r | 1587.17 |
| HS_from_MQbrain_171_r | 1586.13 |
| HS_159_r | 1586.00 |
| HS_from_MQbrain_605_r | 1578.33 |
| HS_from_MQbrain_147_r | 1577.59 |
| HS_from_MQbrain_1620_l | 1575.67 |
| HS_from_MQbrain_362_l | 1574.49 |
| HS_from_MQbrain_736_l | 1569.80 |
| HS_287_l | 1563.84 |
| HS_from_MQbrain_1229_r | 1561.04 |
| HSbrain_393_r | 1557.67 |
| HS_from_MQbrain_1715_l | 1557.21 |
| HSbrain_720_r | 1555.81 |
| HS_83_l | 1555.47 |
| HSbrain_363_r | 1555.01 |
| HSbrain_716_l | 1552.16 |
| HSbrain_430_r | 1550.92 |
| HS_from_MQbrain_789_l | 1550.08 |
| HSbrain_593_r | 1548.50 |
| HSbrain_735_l | 1546.66 |
| HS_from_MQbrain_928_r | 1544.69 |
| HS_from_MQbrain_1210_l | 1544.01 |
| HS_from_MQbrain_1427_l | 1542.86 |
| HSbrain_554_l | 1540.21 |
| HSbrain_712_l | 1539.81 |
| HS_51_l | 1538.70 |
| HS_from_MQbrain_1043_r | 1537.86 |
| HS_12_l | 1532.90 |
| HS_from_MQbrain_609_l | 1532.45 |
| HS_5_l | 1531.44 |
| HS_206_l | 1529.48 |
| HS_from_MQbrain_921_l | 1527.75 |
| HSbrain_1263_r | 1516.38 |
| HSbrain_348_r | 1515.59 |
| HS_79_r | 1513.19 |
| HSbrain_709_l | 1509.68 |
| Hsd_from_Mmd_258_l | 1508.19 |
| HSbrain_626_l | 1507.69 |
| HS_from_MM_305_l | 1507.02 |
| HSbrain_226_r | 1502.51 |
| Hsd_from_Mmd_518_r | 1496.12 |
| HS_from_MQbrain_1423_l | 1495.46 |
| HS_28_r | 1489.19 |
| HS_from_MQbrain_886_r | 1484.58 |
| HS_from_MQbrain_955_l | 1479.35 |
| HSbrain_560_l | 1478.82 |
| HSbrain_103_l | 1477.99 |
| HS_from_MQbrain_1701_l | 1476.79 |
| HS_165_r | 1472.96 |
| HS_from_MQbrain_239_r | 1469.78 |
| HS_from_MQbrain_74_r | 1469.53 |
| HSbrain_525_l | 1461.75 |
| HS_from_MQbrain_738_l | 1459.42 |
| HS_230_r | 1455.02 |
| HS_from_MQbrain_743_l | 1453.79 |
| HS_from_MQbrain_270_l | 1453.68 |
| HS_from_MQbrain_1624_l | 1452.35 |
| HS_from_MQbrain_1621_r | 1451.21 |
| HSbrain_193_l | 1450.35 |
| Hsd_63_r | 1446.53 |
| HS_from_MQbrain_709_r | 1444.22 |
| HSbrain_1364_l | 1439.39 |
| HS_from_MQbrain_1877_r | 1438.74 |
| HS_94_r | 1437.61 |
| HS_from_MQbrain_524_r | 1432.01 |
| HS_233_r | 1428.47 |
| Hsd_from_Mmd_416_l | 1427.90 |
| HS_from_PT_219_l | 1425.59 |
| HS_from_PT_299_l | 1425.10 |
| HS_21_l | 1423.50 |
| HS_from_MQbrain_1845_r | 1421.11 |
| HS_from_MQbrain_1670_r | 1419.08 |
| HSbrain_632_r | 1418.09 |
| HS_from_MQbrain_1484_l | 1417.92 |
| HSbrain_771_l | 1417.26 |
| HS_from_MQbrain_495_l | 1414.11 |
| HS_from_PT_20_r | 1411.59 |
| Hsd_from_Mmd_330_l | 1411.51 |
| HS_35_l | 1405.47 |
| Hsd_29_r | 1405.38 |
| HS_from_MQbrain_399_l | 1395.01 |
| HS_from_MM_79_l | 1394.17 |
| HSbrain_154_l | 1393.99 |
| HS_from_PT_339_l | 1393.25 |
| HS_from_PT_285_l | 1392.11 |
| HS_from_MQbrain_917_r | 1391.24 |
| HS_18_l | 1388.20 |
| HS_from_MQbrain_1223_l | 1386.53 |
| HS_from_MQbrain_288_l | 1382.60 |
| HS_from_MQbrain_1263_l | 1379.79 |
| HS_74_l | 1379.24 |
| HS_222_r | 1375.01 |
| HSbrain_420_r | 1374.92 |
| HS_from_MQbrain_1529_r | 1369.95 |
| HSbrain_869_r | 1366.89 |
| HS_from_PT_27_r | 1359.52 |
| HS_from_MQbrain_207_l | 1356.12 |
| HS_from_MM_67_r | 1345.12 |
| Hsd_from_Mmd_31_l | 1340.93 |
| HS_from_PT_3_r | 1338.70 |
| HSbrain_610_r | 1336.12 |
| Hsd_from_Mmd_97_r | 1333.28 |
| HS_from_MM_117_l | 1320.64 |
| HS_from_MQbrain_1802_r | 1317.20 |
| HSbrain_1262_l | 1314.11 |
| HS_289_l | 1312.22 |
| HS_from_MQbrain_271_r | 1310.78 |
| HSbrain_1325_l | 1308.23 |
| Hsd_32_l | 1301.15 |
| HS_from_MQbrain_450_l | 1300.40 |
| HS_from_MQbrain_1390_l | 1300.35 |
| HS_from_MQbrain_1299_r | 1298.24 |
| HS_from_MQbrain_1747_r | 1297.11 |
| HS_from_MQbrain_1627_l | 1294.42 |
| HSbrain_287_l | 1288.36 |
| HSbrain_428_l | 1287.91 |
| HS_77_l | 1287.06 |
| HSbrain_47_l | 1279.88 |
| HS_from_MM_91_r | 1279.67 |
| HS_from_MQbrain_1897_r | 1275.07 |
| HSbrain_220_l | 1272.15 |
| HSbrain_19_r | 1270.76 |
| HSbrain_815_r | 1270.24 |
| HSbrain_636_l | 1264.65 |
| HS_from_MQbrain_1339_l | 1264.36 |
| HSbrain_469_r | 1263.69 |
| HS_from_MQbrain_1167_l | 1262.46 |
| HSbrain_207_r | 1260.98 |
| HS_from_PT_101_l | 1260.61 |
| HS_from_MQbrain_1394_r | 1251.96 |
| HSbrain_1360_l | 1251.79 |
| HSbrain_107_l | 1246.58 |
| Hsd_31_l | 1246.08 |
| HS_from_MQbrain_1036_r | 1242.65 |
| HS_from_MQbrain_691_r | 1239.61 |
| HS_from_MQbrain_280_r | 1239.25 |
| HSbrain_388_r | 1236.45 |
| HS_from_MQbrain_318_l | 1236.16 |
| HS_from_MQbrain_828_l | 1227.90 |
| HSbrain_750_l | 1226.60 |
| HS_from_MQbrain_1855_l | 1224.15 |
| HS_from_MQbrain_560_l | 1223.27 |
| HSbrain_316_l | 1216.28 |
| HS_290_l | 1215.51 |
| HSbrain_455_r | 1213.86 |
| HS_from_MQbrain_707_l | 1212.83 |
| HS_121_r | 1207.85 |
| Hsd_69_l | 1207.06 |
| HS_from_MQbrain_1501_r | 1204.24 |
| Hsd_from_Mmd_47_r | 1203.10 |
| HSbrain_92_l | 1202.44 |
| HSbrain_794_l | 1200.68 |
| HS_from_PT_220_l | 1192.62 |
| HS_from_MQbrain_497_l | 1192.32 |
| HS_from_MQbrain_739_r | 1190.70 |
| Hsd_from_Mmd_128_r | 1187.96 |
| Hsd_from_Mmd_60_r | 1184.44 |
| HSbrain_217_l | 1184.23 |
| HSbrain_687_l | 1178.66 |
| HS_150_l | 1178.40 |
| HS_from_MQbrain_238_l | 1176.02 |
| HS_from_MQbrain_559_l | 1174.33 |
| HSbrain_153_l | 1172.80 |
| HS_from_PT_28_l | 1172.38 |
| HS_from_MQbrain_272_l | 1172.26 |
| HS_from_MQbrain_580_l | 1172.02 |
| HS_from_MQbrain_640_l | 1170.17 |
| HS_from_MQbrain_1471_l | 1169.33 |
| HS_from_MQbrain_1388_l | 1165.24 |
| HSbrain_1363_l | 1163.09 |
| HS_from_MQbrain_406_r | 1158.97 |
| HS_from_MQbrain_882_r | 1150.61 |
| Hsd_from_Mmd_101_r | 1150.48 |
| Hsd_from_Mmd_98_r | 1148.86 |
| HS_126_r | 1148.46 |
| Hsd_from_Mmd_256_l | 1147.57 |
| HS_from_MQbrain_319_r | 1144.78 |
| HS_from_MQbrain_29_l | 1136.64 |
| Hsd_from_Mmd_471_l | 1134.05 |
| HSbrain_23_r | 1133.49 |
| HS_from_MQbrain_54_r | 1128.30 |
| HSbrain_50_l | 1128.11 |
| HS_from_MQbrain_1544_r | 1128.06 |
| HS_from_MQbrain_943_l | 1120.64 |
| HS_58_l | 1112.12 |
| HS_from_PT_43_l | 1108.55 |
| HSbrain_268_l | 1107.97 |
| HS_from_MQbrain_1298_r | 1106.93 |
| HS_125_l | 1105.97 |
| HSbrain_691_l | 1101.52 |
| HS_from_MQbrain_1695_r | 1101.10 |
| HS_from_PT_70_r | 1095.93 |
| HS_40_r | 1094.71 |
| HS_from_MM_306_r | 1086.82 |
| HS_from_MQbrain_1366_l | 1085.63 |
| HS_from_MQbrain_1888_r | 1084.94 |
| HSbrain_204_r | 1075.46 |
| HSbrain_1431_l | 1073.27 |
| HSbrain_1426_l | 1073.12 |
| Hsd_71_l | 1072.31 |
| HS_from_MQbrain_645_l | 1070.63 |
| HS_from_MQbrain_211_l | 1070.30 |
| HSbrain_373_l | 1070.01 |
| HSbrain_594_l | 1061.87 |
| HS_from_MQbrain_1660_r | 1055.20 |
| HSbrain_527_r | 1052.64 |
| HSbrain_652_l | 1052.64 |
| HS_238_r | 1049.82 |
| HSbrain_151_l | 1043.22 |
| HSbrain_515_r | 1042.74 |
| HSbrain_570_l | 1042.70 |
| HS_from_MQbrain_1152_l | 1041.31 |
| HSbrain_724_r | 1041.29 |
| HSbrain_419_l | 1039.00 |
| Hsd_from_Mmd_480_r | 1038.29 |
| Hsd_38_r | 1037.46 |
| HS_from_MQbrain_152_r | 1036.10 |
| HSbrain_624_l | 1034.54 |
| HS_from_MQbrain_884_r | 1034.38 |
| HS_149_l | 1034.35 |
| HS_from_MQbrain_911_r | 1032.49 |
| Hsd_from_Mmd_477_r | 1031.55 |
| HS_from_MQbrain_671_r | 1023.79 |
| HS_from_MQbrain_723_r | 1023.31 |
| HSbrain_409_r | 1021.91 |
| HSbrain_475_r | 1021.77 |
| HSbrain_584_r | 1020.98 |
| HS_from_MQbrain_1225_l | 1017.03 |
| HS_from_PT_163_r | 1015.09 |
| HSbrain_311_r | 1013.48 |
| HS_from_MQbrain_731_r | 1011.65 |
| HS_from_MQbrain_67_l | 1010.93 |
| Hsd_58_l | 1000.86 |
| HS_193_l | 1000.86 |
| HS_from_MQbrain_421_r | 1000.28 |
| HS_57_l | 1000.27 |
| HS_from_PT_55_l | 994.19 |
| HSbrain_606_l | 993.78 |
| HS_from_MQbrain_1623_r | 992.29 |
| HS_from_MQbrain_1031_r | 987.03 |
| HS_from_MM_112_r | 985.98 |
| HS_from_MQbrain_1372_l | 983.18 |
| HS_from_MQbrain_120_r | 978.06 |
| HS_from_MM_171_r | 971.69 |
| HSbrain_1362_l | 962.43 |
| HS_from_MQbrain_1637_r | 961.67 |
| HS_from_MQbrain_1729_r | 957.70 |
| HS_from_MQbrain_83_l | 956.01 |
| Hsd_from_Mmd_28_r | 952.22 |
| HS_29_l | 950.02 |
| HS_from_MQbrain_1721_l | 944.26 |
| HSbrain_75_l | 940.56 |
| Hsd_from_Mmd_180_l | 940.08 |
| HS_from_MQbrain_1376_l | 937.34 |
| HS_from_MQbrain_1454_l | 934.33 |
| HSbrain_252_r | 933.92 |
| HS_116_l | 933.78 |
| HS_from_MQbrain_1537_l | 932.97 |
| HSbrain_372_l | 930.27 |
| HS_from_MQbrain_1717_l | 926.22 |
| HS_from_MQbrain_331_l | 925.79 |
| HS_285_r | 925.13 |
| HSbrain_108_l | 923.37 |
| HS_from_MQbrain_1810_l | 923.33 |
| HSbrain_115_r | 922.70 |
| HS_from_MQbrain_1545_l | 916.47 |
| HSbrain_104_l | 915.09 |
| HS_from_MQbrain_1625_r | 907.04 |
| HSbrain_804_r | 906.10 |
| HS_from_PT_94_r | 904.04 |
| HS_from_MQbrain_386_l | 903.16 |
| HSbrain_260_l | 900.12 |
| HS_from_MQbrain_648_l | 898.68 |
| HS_196_l | 898.20 |
| HSbrain_559_l | 897.17 |
| Hsd_from_Mmd_470_l | 891.50 |
| HS_from_MQbrain_1280_l | 891.24 |
| HSbrain_275_l | 890.43 |
| HS_from_MQbrain_1193_r | 887.95 |
| HSbrain_514_l | 885.67 |
| HS_from_MQbrain_2669_r | 885.25 |
| HS_from_MQbrain_266_l | 878.84 |
| HS_from_PT_229_l | 878.81 |
| HS_from_MQbrain_447_l | 876.75 |
| HS_from_MM_73_l | 871.42 |
| HSbrain_105_l | 870.23 |
| HS_26_l | 867.88 |
| HS_from_PT_25_l | 864.39 |
| HS_107_l | 854.00 |
| HS_from_MQbrain_1619_r | 851.82 |
| HS_from_MQbrain_1150_r | 846.73 |
| HSbrain_715_r | 843.92 |
| HS_from_MQbrain_6_l | 840.88 |
| HS_from_MQbrain_477_l | 840.85 |
| HS_from_MQbrain_1794_r | 837.36 |
| Hsd_from_Mmd_529_l | 835.75 |
| HS_from_MQbrain_14_r | 835.72 |
| HS_from_MQbrain_1112_r | 833.40 |
| HS_from_MQbrain_338_r | 832.89 |
| HSbrain_618_r | 828.32 |
| HS_from_MQbrain_693_r | 828.02 |
| HSbrain_1400_r | 824.35 |
| HS_from_MQbrain_1185_r | 820.62 |
| HS_from_MQbrain_291_l | 819.28 |
| HSbrain_599_l | 814.14 |
| HSbrain_693_l | 812.62 |
| HS_17_r | 811.30 |
| HSbrain_537_r | 810.14 |
| HS_from_MQbrain_528_l | 809.91 |
| HS_from_MQbrain_1549_r | 806.18 |
| HS_from_MQbrain_1307_l | 804.44 |
| HS_from_MQbrain_278_l | 802.79 |
| HSbrain_470_l | 801.49 |
| HS_from_MQbrain_1865_r | 801.45 |
| HS_from_MQbrain_85_r | 801.24 |
| HS_from_PT_280_r | 797.48 |
| HSbrain_63_l | 795.52 |
| HS_56_r | 793.43 |
| HS_from_MQbrain_1546_r | 792.24 |
| HS_from_MQbrain_1675_r | 791.80 |
| HS_from_MQbrain_71_r | 790.88 |
| HSbrain_634_l | 788.90 |
| HSbrain_812_r | 788.07 |
| HSbrain_595_r | 785.74 |
| HS_from_MQbrain_527_l | 784.29 |
| HS_from_MQbrain_1547_l | 783.72 |
| HS_from_MQbrain_1259_l | 780.76 |
| HSbrain_513_r | 778.63 |
| HS_277_l | 776.25 |
| HS_from_MQbrain_903_r | 771.84 |
| HS_from_MQbrain_282_l | 762.12 |
| HS_from_MQbrain_782_r | 761.75 |
| HS_from_MM_70_r | 759.89 |
| HS_from_MQbrain_861_l | 759.27 |
| Hsd_from_Mmd_129_r | 757.25 |
| HS_from_MQbrain_258_l | 756.39 |
| Hsd_42_r | 755.65 |
| HS_from_MQbrain_1231_r | 751.70 |
| HSbrain_185_l | 751.55 |
| HS_245_r | 751.26 |
| HSbrain_796_l | 746.21 |
| HS_from_PT_95_r | 744.48 |
| HS_from_MQbrain_1082_l | 744.36 |
| HSbrain_164_r | 743.73 |
| HS_from_MQbrain_313_r | 741.59 |
| HS_6_l | 738.76 |
| Hsd_62_l | 733.75 |
| HS_from_MQbrain_86_l | 728.72 |
| HS_from_MQbrain_308_l | 725.31 |
| HS_from_MQbrain_2700_l | 724.46 |
| Hsd_from_Mmd_155_r | 722.15 |
| HS_from_MQbrain_145_l | 715.93 |
| HS_114_l | 715.10 |
| HS_from_MQbrain_1684_l | 714.03 |
| HSbrain_741_l | 713.47 |
| HS_from_MQbrain_825_l | 711.19 |
| HS_from_MM_210_r | 707.87 |
| HS_54_l | 705.25 |
| HS_136_l | 700.84 |
| HS_from_MQbrain_475_r | 695.96 |
| HS_from_MQbrain_1190_r | 695.77 |
| HS_from_MQbrain_1605_r | 695.45 |
| HS_from_MQbrain_1283_l | 692.85 |
| HS_from_MQbrain_150_l | 691.44 |
| HS_from_PT_41_l | 688.41 |
| HS_from_MQbrain_1752_r | 687.03 |
| Hsd_70_l | 685.00 |
| HS_279_l | 682.84 |
| HSbrain_175_l | 675.74 |
| HSbrain_169_l | 675.70 |
| HSbrain_464_l | 674.35 |
| HS_44_l | 666.78 |
| HSbrain_665_l | 664.02 |
| HS_from_MQbrain_1754_l | 661.49 |
| HS_from_MQbrain_1382_l | 661.03 |
| HSbrain_96_l | 656.78 |
| HSbrain_317_l | 654.52 |
| HSbrain_102_r | 653.85 |
| HS_from_MQbrain_654_r | 652.78 |
| HS_from_MM_195_r | 648.55 |
| HSbrain_202_r | 646.85 |
| HSbrain_1399_l | 640.61 |
| HSbrain_168_l | 636.93 |
| HSbrain_330_l | 636.49 |
| HS_291_l | 635.36 |
| HS_from_MQbrain_63_l | 634.91 |
| HS_118_l | 634.82 |
| HS_from_PT_255_l | 634.81 |
| HS_from_MQbrain_684_l | 630.77 |
| HS_from_MQbrain_1402_l | 628.81 |
| HS_from_MQbrain_883_r | 626.09 |
| HS_from_MQbrain_1655_l | 625.58 |
| HSbrain_767_r | 623.64 |
| HSbrain_54_l | 619.45 |
| HS_from_MQbrain_268_l | 614.25 |
| HS_from_MQbrain_1344_l | 612.60 |
| HSbrain_130_l | 610.32 |
| HSbrain_134_l | 605.11 |
| HSbrain_267_l | 604.78 |
| HS_from_MQbrain_918_l | 604.37 |
| HS_from_MQbrain_933_l | 604.15 |
| HS_from_MQbrain_590_r | 603.52 |
| HS_from_MQbrain_363_l | 601.52 |
| HS_from_MQbrain_873_r | 599.55 |
| HSbrain_392_l | 599.45 |
| HS_256_r | 597.95 |
| HS_from_PT_250_r | 597.57 |
| HS_128_r | 595.80 |
| HSbrain_243_l | 593.95 |
| HSbrain_879_r | 591.43 |
| HSbrain_366_l | 591.25 |
| HSbrain_528_r | 590.05 |
| HSbrain_743_l | 589.95 |
| HS_24_r | 589.66 |
| HS_from_MQbrain_1239_l | 588.81 |
| HSbrain_321_r | 587.71 |
| HS_from_MM_65_l | 584.19 |
| HS_53_r | 582.76 |
| HSbrain_150_r | 580.85 |
| HS_244_l | 580.50 |
| HSbrain_522_l | 577.78 |
| HS_from_PT_294_l | 574.91 |
| HSbrain_280_l | 571.68 |
| HS_from_MQbrain_1776_l | 568.88 |
| HS_from_MQbrain_485_r | 568.38 |
| HS_from_MQbrain_317_l | 566.36 |
| HS_from_MQbrain_1143_l | 566.08 |
| HS_70_l | 563.85 |
| HS_from_MQbrain_751_r | 557.33 |
| HSbrain_622_l | 556.70 |
| HSbrain_752_l | 556.10 |
| HS_from_MQbrain_260_r | 554.62 |
| HSbrain_722_l | 554.56 |
| HS_from_MQbrain_1266_r | 554.29 |
| HSbrain_88_r | 553.13 |
| HSbrain_386_l | 552.41 |
| Hsd_18_l | 551.25 |
| HS_131_r | 546.58 |
| Hsd_17_l | 546.42 |
| HS_from_PT_190_r | 545.93 |
| HSbrain_749_r | 545.39 |
| HS_from_MQbrain_962_l | 543.74 |
| HS_from_MQbrain_541_r | 539.50 |
| HSbrain_110_r | 538.55 |
| HSbrain_520_l | 536.06 |
| HS_from_MQbrain_840_l | 534.32 |
| HSbrain_639_r | 526.95 |
| HSbrain_286_l | 526.85 |
| Hsd_from_Mmd_418_r | 526.84 |
| HS_from_MQbrain_878_l | 522.80 |
| HS_from_MM_121_r | 522.13 |
| HS_246_l | 518.00 |
| HS_from_PT_283_l | 517.37 |
| HS_208_r | 514.20 |
| HS_from_MQbrain_708_r | 510.81 |
| HS_from_MM_350_l | 510.04 |
| HSbrain_186_r | 507.79 |
| HSbrain_1424_l | 507.68 |
| HSbrain_547_r | 506.78 |
| HS_from_MQbrain_347_l | 505.50 |
| HS_from_MQbrain_819_l | 505.30 |
| Hsd_from_Mmd_511_l | 503.35 |
| HS_109_l | 502.50 |
| Hsd_from_Mmd_424_r | 498.63 |
| HS_from_MQbrain_599_l | 495.60 |
| HS_from_MQbrain_1328_l | 491.37 |
| HSbrain_694_r | 487.83 |
| HS_275_l | 483.08 |
| HS_from_PT_56_r | 482.34 |
| HS_from_MQbrain_374_r | 481.44 |
| HSbrain_1408_r | 481.04 |
| HS_from_MQbrain_499_r | 480.23 |
| HS_from_MQbrain_1773_l | 479.94 |
| HSbrain_292_l | 478.77 |
| HS_from_MQbrain_960_l | 474.98 |
| HS_from_MQbrain_818_l | 473.82 |
| HS_from_MQbrain_1493_r | 472.59 |
| HSbrain_1_l | 471.82 |
| HSbrain_329_l | 471.00 |
| Hsd_from_Mmd_456_l | 470.27 |
| HS_249_l | 469.47 |
| HS_from_MQbrain_1668_l | 467.89 |
| HSbrain_218_r | 466.80 |
| HS_134_r | 465.99 |
| HS_248_l | 464.57 |
| HSbrain_95_l | 464.25 |
| HS_from_MQbrain_1046_r | 463.30 |
| HS_from_MQbrain_92_l | 462.65 |
| HS_from_MQbrain_1295_r | 460.64 |
| HS_123_l | 456.64 |
| HS_76_l | 455.93 |
| HS_from_MQbrain_1213_l | 455.52 |
| HS_from_MQbrain_1475_l | 452.53 |
| HS_from_MQbrain_902_r | 452.27 |
| HSbrain_1269_r | 451.53 |
| Hsd_105_r | 448.68 |
| HS_from_MQbrain_131_r | 447.53 |
| HSbrain_291_l | 447.40 |
| HSbrain_675_l | 447.31 |
| HSbrain_710_r | 446.97 |
| HSbrain_85_l | 443.76 |
| HS_from_MQbrain_47_l | 442.32 |
| HS_304_r | 442.22 |
| Hsd_from_Mmd_303_r | 441.07 |
| HS_from_MQbrain_574_l | 439.75 |
| HS_from_MQbrain_401_r | 438.46 |
| Hsd_107_r | 438.16 |
| HS_from_MM_216_r | 435.69 |
| Hsd_from_Mmd_277_r | 435.62 |
| HS_from_MQbrain_1184_r | 434.90 |
| HSbrain_586_r | 433.97 |
| HS_from_MQbrain_1507_l | 429.93 |
| HS_from_MQbrain_969_r | 424.82 |
| HSbrain_319_r | 420.84 |
| HS_from_MQbrain_333_r | 417.85 |
| HS_from_MQbrain_1601_l | 417.52 |
| HS_from_MQbrain_1807_l | 413.66 |
| HSbrain_293_r | 409.42 |
| HS_115_l | 407.53 |
| HS_from_MQbrain_1706_l | 404.70 |
| HSbrain_568_l | 403.67 |
| HSbrain_415_l | 403.23 |
| Hsd_from_Mmd_305_l | 399.55 |
| HS_from_MQbrain_538_l | 396.70 |
| HSbrain_144_r | 395.32 |
| HS_from_MM_58_r | 394.91 |
| HS_from_PT_231_r | 392.52 |
| HS_from_MQbrain_1348_r | 391.28 |
| HSbrain_503_r | 388.85 |
| HS_from_MQbrain_340_r | 387.00 |
| Hsd_73_r | 386.32 |
| HS_from_MQbrain_1215_r | 385.09 |
| HS_from_MQbrain_830_l | 384.93 |
| HS_from_MQbrain_774_r | 383.95 |
| HS_from_MQbrain_1552_r | 383.47 |
| HS_from_MQbrain_1593_l | 382.90 |
| HS_from_MQbrain_1678_l | 380.24 |
| HSbrain_474_l | 379.24 |
| HSbrain_355_r | 374.26 |
| HS_31_l | 372.30 |
| HS_from_MM_6_l | 372.25 |
| HS_from_MQbrain_1398_l | 371.68 |
| Hsd_from_Mmd_147_r | 370.58 |
| HS_from_MQbrain_788_r | 370.49 |
| HSbrain_653_r | 370.44 |
| HSbrain_255_l | 369.89 |
| HSbrain_516_r | 365.85 |
| HS_from_MQbrain_845_r | 365.11 |
| HS_from_MQbrain_1289_l | 364.25 |
| Hsd_from_Mmd_123_l | 362.29 |
| HS_from_MQbrain_1443_r | 361.33 |
| HSbrain_575_l | 360.85 |
| HS_from_MQbrain_721_r | 358.12 |
| HS_from_MQbrain_1768_l | 355.91 |
| Hsd_from_Mmd_49_r | 353.94 |
| HS_1_r | 353.94 |
| HS_from_MQbrain_1090_l | 351.45 |
| HSbrain_133_l | 350.07 |
| HS_from_MQbrain_1379_l | 349.15 |
| HS_from_MQbrain_409_l | 349.03 |
| HS_from_MQbrain_750_r | 348.17 |
| HS_242_r | 348.01 |
| HS_from_MQbrain_97_r | 346.64 |
| HSbrain_385_l | 344.94 |
| HS_from_MQbrain_1885_r | 344.16 |
| HS_from_MQbrain_783_l | 343.37 |
| HSbrain_597_r | 342.91 |
| HSbrain_118_r | 340.48 |
| HS_from_MQbrain_669_l | 340.11 |
| HS_from_MQbrain_755_r | 339.47 |
| HS_from_MQbrain_567_l | 338.91 |
| HS_from_PT_105_l | 336.79 |
| HSbrain_635_r | 336.11 |
| HS_from_MQbrain_1703_l | 332.45 |
| HS_from_MQbrain_592_l | 331.90 |
| HSbrain_1336_r | 329.26 |
| HS_from_MQbrain_1318_l | 327.82 |
| Hsd_from_Mmd_301_l | 326.72 |
| HSbrain_251_l | 326.54 |
| HSbrain_162_l | 322.91 |
| HSbrain_93_l | 322.47 |
| Hsd_from_Mmd_450_r | 319.66 |
| HS_from_MQbrain_1712_l | 318.49 |
| HS_from_MQbrain_940_r | 317.39 |
| HS_from_PT_328_l | 317.19 |
| HS_15_l | 316.41 |
| HSbrain_488_l | 313.47 |
| HS_from_MQbrain_1673_r | 313.25 |
| HS_from_MQbrain_981_l | 311.17 |
| HS_from_MQbrain_413_l | 306.41 |
| HS_from_MQbrain_801_l | 305.81 |
| HSbrain_582_r | 304.05 |
| HS_from_MQbrain_862_l | 303.62 |
| HS_from_MQbrain_284_r | 302.38 |
| HS_from_MQbrain_178_l | 300.55 |
| HS_from_MQbrain_1393_l | 298.80 |
| HS_87_r | 297.76 |
| HS_from_MQbrain_1831_r | 297.75 |
| HS_from_MQbrain_18_l | 297.60 |
| HS_from_MQbrain_84_l | 297.02 |
| HSbrain_353_r | 294.70 |
| HSbrain_121_l | 293.17 |
| HS_4_r | 293.08 |
| HS_from_MQbrain_1875_l | 292.51 |
| HS_from_MQbrain_706_l | 291.66 |
| HS_from_MQbrain_149_l | 291.54 |
| HS_303_r | 290.92 |
| Hsd_1_l | 290.92 |
| Hsd_from_Mmd_488_l | 288.56 |
| HS_from_MQbrain_1303_r | 288.49 |
| HS_from_MQbrain_1565_r | 286.84 |
| HS_from_PT_325_r | 286.73 |
| HS_from_MM_179_l | 286.55 |
| HSbrain_300_r | 286.46 |
| HSbrain_51_r | 284.78 |
| HSbrain_383_l | 283.64 |
| HSbrain_698_l | 283.24 |
| HS_from_MQbrain_1719_l | 282.17 |
| HSbrain_136_l | 282.00 |
| HSbrain_301_r | 281.80 |
| HSbrain_288_l | 280.92 |
| HS_268_r | 279.89 |
| HS_from_MQbrain_659_l | 278.81 |
| HS_from_MQbrain_1784_r | 278.43 |
| HSbrain_723_l | 277.73 |
| HS_from_MQbrain_913_r | 276.93 |
| HSbrain_176_l | 276.55 |
| HS_from_MQbrain_191_l | 276.03 |
| HSbrain_364_l | 275.31 |
| HS_152_l | 271.53 |
| HSbrain_421_r | 271.48 |
| HS_from_MQbrain_490_r | 269.59 |
| HS_from_MQbrain_1051_l | 269.40 |
| HSbrain_1437_l | 266.70 |
| HS_from_MQbrain_28_r | 265.68 |
| HSbrain_564_r | 265.22 |
| HSbrain_41_l | 264.89 |
| HS_from_MQbrain_1551_r | 262.98 |
| HSbrain_61_r | 261.35 |
| HSbrain_534_l | 261.28 |
| HSbrain_616_r | 260.24 |
| HS_from_MQbrain_661_l | 259.94 |
| HSbrain_248_l | 258.90 |
| Hsd_65_r | 258.71 |
| HS_from_MQbrain_1708_l | 257.81 |
| HS_from_MQbrain_1664_r | 257.46 |
| HSbrain_762_r | 257.10 |
| HSbrain_590_r | 254.85 |
| Hsd_64_r | 254.29 |
| HSbrain_403_r | 254.03 |
| HS_from_MQbrain_388_r | 253.35 |
| HS_from_MQbrain_868_l | 251.95 |
| HS_from_MQbrain_2633_l | 251.66 |
| HS_from_MQbrain_1456_l | 251.46 |
| HS_from_MQbrain_1010_l | 250.06 |
| HS_from_MQbrain_315_l | 248.59 |
| HS_from_MQbrain_1340_l | 248.35 |
| Hsd_from_Mmd_530_r | 248.30 |
| Hsd_77_r | 247.78 |
| HS_247_l | 247.65 |
| HS_from_PT_300_l | 245.35 |
| HSbrain_183_r | 245.06 |
| HSbrain_74_l | 244.28 |
| HS_from_MM_299_r | 243.94 |
| HS_from_MQbrain_965_l | 243.53 |
| HSbrain_793_r | 243.17 |
| HS_from_MQbrain_1374_l | 242.85 |
| HS_186_r | 242.25 |
| HSbrain_49_r | 241.32 |
| HS_from_MQbrain_1561_l | 240.38 |
| HS_from_MQbrain_1288_l | 238.85 |
| HS_from_MQbrain_1279_r | 238.31 |
| HS_from_MQbrain_629_l | 237.88 |
| HSbrain_1305_l | 237.71 |
| HS_from_MQbrain_1745_r | 237.34 |
| HS_146_l | 235.71 |
| HS_from_MQbrain_1315_l | 234.79 |
| HSbrain_776_r | 233.87 |
| Hsd_from_Mmd_189_l | 233.41 |
| HS_from_MQbrain_1433_r | 232.94 |
| HSbrain_608_r | 232.74 |
| HSbrain_751_l | 232.40 |
| HS_from_PT_211_l | 230.31 |
| HS_from_MQbrain_41_r | 229.85 |
| HSbrain_768_r | 229.66 |
| HSbrain_613_l | 229.34 |
| HS_from_MQbrain_504_l | 229.11 |
| HSbrain_155_l | 228.39 |
| HS_from_MQbrain_1227_l | 228.29 |
| HS_from_PT_207_r | 227.55 |
| HSbrain_784_r | 227.25 |
| HS_from_MQbrain_343_l | 224.21 |
| HS_from_MQbrain_405_l | 224.19 |
| HS_124_l | 223.23 |
| Hsd_52_l | 223.10 |
| HS_from_MQbrain_1175_r | 221.91 |
| HS_164_l | 221.00 |
| HS_22_l | 220.60 |
| HSbrain_303_l | 220.32 |
| HS_from_MQbrain_1212_r | 220.09 |
| Hsd_from_Mmd_483_l | 219.16 |
| Hsd_from_Mmd_20_l | 216.97 |
| HS_from_MQbrain_951_l | 216.15 |
| HS_from_MQbrain_1555_l | 215.81 |
| HSbrain_1414_l | 214.70 |
| HS_from_MQbrain_349_l | 214.68 |
| HS_from_MQbrain_1055_r | 214.02 |
| HS_from_MQbrain_414_l | 212.24 |
| HSbrain_65_r | 211.29 |
| HS_from_MQbrain_492_l | 210.90 |
| HS_from_MQbrain_948_r | 210.86 |
| Hsd_from_Mmd_145_l | 209.51 |
| HSbrain_126_l | 208.87 |
| HSbrain_359_r | 207.52 |
| HS_from_MQbrain_128_r | 205.98 |
| Hsd_from_Mmd_353_l | 205.36 |
| HS_from_PT_256_l | 203.51 |
| HS_from_MQbrain_1535_l | 203.26 |
| HS_86_l | 202.01 |
| HS_from_MQbrain_806_l | 201.32 |
| HS_from_MQbrain_1395_r | 200.62 |
| HSbrain_142_l | 200.51 |
| HS_from_PT_146_l | 200.34 |
| HS_from_MQbrain_1608_l | 200.30 |
| HS_from_MQbrain_1744_r | 199.26 |
| HS_from_MQbrain_154_l | 198.44 |
| Hsd_19_l | 198.35 |
| HS_283_r | 197.92 |
| HSbrain_369_r | 197.24 |
| HS_from_MQbrain_650_r | 195.56 |
| HS_from_MQbrain_630_l | 195.03 |
| HSbrain_124_l | 194.72 |
| HS_91_r | 193.84 |
| HS_258_l | 193.39 |
| HS_from_MQbrain_922_r | 192.40 |
| HS_112_l | 192.23 |
| HS_from_MQbrain_251_r | 190.83 |
| HSbrain_362_l | 190.33 |
| HSbrain_637_l | 190.08 |
| HS_from_MQbrain_1590_l | 189.53 |
| HSbrain_441_l | 189.11 |
| HS_from_MQbrain_350_l | 188.59 |
| HS_from_MQbrain_1718_l | 188.57 |
| HS_from_MQbrain_1573_l | 188.47 |
| HSbrain_1210_r | 188.45 |
| HS_from_PT_291_l | 188.08 |
| HS_from_MM_44_l | 187.87 |
| HS_from_MQbrain_472_r | 187.77 |
| HS_143_l | 185.71 |
| HS_from_MQbrain_165_l | 185.39 |
| Hsd_from_Mmd_246_r | 185.28 |
| HSbrain_1421_r | 185.02 |
| HSbrain_35_r | 185.02 |
| HS_34_r | 182.83 |
| HS_from_MQbrain_467_l | 182.78 |
| Hsd_from_Mmd_93_l | 182.17 |
| HSbrain_785_r | 181.62 |
| HSbrain_117_r | 181.24 |
| HS_88_l | 181.19 |
| HS_from_MQbrain_1346_l | 180.35 |
| Hsd_from_Mmd_91_r | 178.23 |
| Hsd_87_r | 177.68 |
| HS_from_MQbrain_38_l | 177.46 |
| HS_from_MQbrain_919_r | 177.21 |
| HS_from_MQbrain_1061_l | 172.98 |
| HS_178_r | 172.77 |
| HS_from_MQbrain_1808_r | 172.48 |
| HS_from_MQbrain_1569_l | 171.53 |
| Hsd_108_r | 171.48 |
| Hsd_74_r | 170.76 |
| HS_from_MQbrain_403_l | 169.68 |
| HS_from_MM_251_l | 168.81 |
| HS_from_MQbrain_658_r | 167.88 |
| HS_from_MQbrain_404_l | 165.99 |
| HS_33_r | 164.78 |
| HSbrain_1066_l | 164.40 |
| HS_from_MQbrain_1028_l | 164.03 |
| HS_156_r | 163.86 |
| HSbrain_459_l | 163.49 |
| HS_from_MQbrain_1088_l | 162.85 |
| HS_from_MQbrain_1558_r | 162.47 |
| HSbrain_620_l | 161.99 |
| HSbrain_37_l | 161.83 |
| HS_from_MQbrain_1260_l | 161.33 |
| HS_from_MQbrain_1795_l | 161.31 |
| HS_from_MQbrain_458_r | 160.80 |
| HSbrain_648_r | 160.66 |
| HS_from_MQbrain_1869_l | 160.25 |
| HS_from_MQbrain_123_r | 158.71 |
| HS_from_MQbrain_931_r | 158.29 |
| HSbrain_484_l | 158.28 |
| Hsd_100_l | 157.67 |
| Hsd_99_r | 157.49 |
| HS_from_MQbrain_867_r | 157.00 |
| Hsd_from_Mmd_141_l | 156.62 |
| Hsd_from_Mmd_371_l | 155.83 |
| HS_from_MQbrain_1459_l | 154.95 |
| HS_from_MQbrain_889_r | 154.79 |
| HS_from_MM_289_r | 153.91 |
| HS_from_MQbrain_562_l | 153.24 |
| HS_from_MM_293_r | 153.22 |
| HS_from_MQbrain_785_l | 152.69 |
| HS_from_MQbrain_1108_r | 152.69 |
| HS_from_MQbrain_936_r | 152.19 |
| HS_from_MQbrain_464_l | 151.75 |
| HSbrain_524_r | 151.72 |
| HS_from_MQbrain_1300_l | 151.72 |
| HS_from_MQbrain_494_l | 151.57 |
| HS_from_MQbrain_327_r | 151.54 |
| HS_from_MM_219_l | 151.42 |
| HSbrain_596_r | 151.36 |
| Hsd_from_Mmd_468_r | 151.35 |
| HSbrain_290_l | 150.85 |
| HS_from_MQbrain_1841_r | 150.14 |
| HS_from_MM_163_r | 149.79 |
| HSbrain_495_l | 149.74 |
| Hsd_from_Mmd_344_r | 149.50 |
| Hsd_from_Mmd_266_r | 148.01 |
| HS_from_MM_175_l | 147.90 |
| HS_from_MM_150_l | 147.44 |
| HS_from_MQbrain_1589_r | 147.20 |
| Hsd_92_l | 146.01 |
| HSbrain_573_l | 145.75 |
| Hsd_from_Mmd_52_r | 145.37 |
| HSbrain_1409_r | 144.95 |
| HS_from_PT_121_r | 144.82 |
| HS_255_l | 144.06 |
| HSbrain_178_l | 143.88 |
| HS_from_MQbrain_482_r | 143.47 |
| HS_from_MQbrain_221_l | 143.40 |
| HS_261_r | 142.25 |
| HS_from_MQbrain_558_r | 141.63 |
| Hsd_from_Mmd_439_l | 141.63 |
| HS_from_MQbrain_427_l | 140.97 |
| HS_from_MQbrain_612_r | 140.83 |
| HS_from_MQbrain_973_l | 140.65 |
| HS_169_l | 138.82 |
| HSbrain_658_r | 138.37 |
| HSbrain_165_l | 137.58 |
| HS_from_MQbrain_360_r | 137.28 |
| HS_from_MM_136_r | 137.03 |
| HS_from_MQbrain_470_l | 136.89 |
| HS_from_MQbrain_1463_l | 136.60 |
| HS_from_MQbrain_741_r | 136.21 |
| HS_from_MQbrain_1790_r | 135.71 |
| HSbrain_203_l | 134.99 |
| HSbrain_360_r | 134.83 |
| HSbrain_27_r | 132.91 |
| HS_from_MQbrain_9_r | 130.51 |
| HS_from_MQbrain_1656_l | 130.22 |
| HS_from_PT_31_r | 130.08 |
| HS_from_MQbrain_369_r | 129.29 |
| HS_from_MQbrain_666_r | 129.05 |
| HS_from_MQbrain_606_l | 128.88 |
| HS_from_MM_202_l | 128.65 |
| Hsd_101_l | 127.99 |
| HS_from_MQbrain_183_l | 126.73 |
| HS_from_MQbrain_2689_l | 125.98 |
| HS_138_l | 125.63 |
| HS_from_MQbrain_56_l | 125.18 |
| HS_from_MQbrain_1822_r | 125.05 |
| Hsd_from_Mmd_501_r | 125.01 |
| HS_2_l | 123.48 |
| HS_from_MQbrain_486_l | 123.37 |
| Hsd_97_l | 123.35 |
| HSbrain_387_r | 123.32 |
| HS_from_MQbrain_1722_l | 121.03 |
| HSbrain_181_r | 120.88 |
| HS_from_MQbrain_383_l | 120.82 |
| HS_from_MQbrain_621_r | 120.69 |
| HS_from_MQbrain_1542_r | 119.21 |
| Hsd_from_Mmd_112_l | 119.21 |
| HSbrain_578_r | 119.12 |
| HS_from_MQbrain_52_r | 118.77 |
| HS_from_MQbrain_1205_r | 118.18 |
| HSbrain_38_r | 118.09 |
| HSbrain_733_l | 117.71 |
| HSbrain_592_r | 117.60 |
| HSbrain_69_r | 117.41 |
| HS_from_MQbrain_663_r | 117.29 |
| HSbrain_502_l | 117.21 |
| Hsd_79_l | 115.62 |
| HS_from_PT_303_l | 115.42 |
| HSbrain_68_r | 115.20 |
| HSbrain_807_r | 114.06 |
| Hsd_11_l | 114.03 |
| HS_from_MM_109_r | 113.69 |
| HSbrain_490_r | 113.20 |
| Hsd_from_Mmd_284_r | 112.69 |
| HS_from_MQbrain_1183_l | 112.54 |
| HS_from_MQbrain_1070_l | 112.16 |
| HS_from_MQbrain_1781_r | 111.29 |
| HS_from_MQbrain_287_r | 110.79 |
| HS_from_MQbrain_591_l | 110.25 |
| Hsd_from_Mmd_245_r | 110.10 |
| HSbrain_729_l | 109.77 |
| HS_232_l | 109.73 |
| Hsd_61_l | 109.69 |
| HS_from_PT_234_l | 109.50 |
| HS_from_MQbrain_304_r | 109.46 |
| HS_from_MQbrain_261_r | 108.55 |
| Hsd_83_r | 107.53 |
| Hsd_from_Mmd_136_r | 107.29 |
| HS_from_MM_319_l | 107.03 |
| Hsd_78_l | 106.73 |
| HS_from_MQbrain_1854_r | 106.61 |
| HSbrain_346_l | 106.26 |
| HSbrain_617_l | 106.18 |
| HS_from_MM_33_r | 106.17 |
| HS_from_MQbrain_1743_r | 106.15 |
| HS_141_l | 105.74 |
| HSbrain_640_r | 105.57 |
| HS_267_r | 105.00 |
| HS_from_MQbrain_1818_r | 104.59 |
| HS_from_MQbrain_1030_l | 104.18 |
| HS_189_l | 103.99 |
| HSbrain_772_l | 103.44 |
| HS_from_MQbrain_1610_r | 102.62 |
| HS_110_l | 102.30 |
| HS_from_MQbrain_841_l | 101.61 |
| HSbrain_1168_l | 101.59 |
| HS_from_MQbrain_1775_r | 101.49 |
| HSbrain_129_r | 101.04 |
| Hsd_from_Mmd_193_l | 100.90 |
| HS_from_MQbrain_1766_r | 100.48 |
| HS_from_MQbrain_2380_r | 100.43 |
| HSbrain_310_l | 100.30 |
| HS_from_MM_273_l | 99.76 |
| Hsd_from_Mmd_270_l | 99.75 |
| HSbrain_436_l | 99.64 |
| HS_from_MM_153_l | 99.43 |
| HS_from_MM_176_r | 98.96 |
| HSbrain_98_l | 98.56 |
| HS_from_MQbrain_1830_r | 97.76 |
| HS_from_MQbrain_1797_r | 97.58 |
| Hsd_109_l | 96.85 |
| HS_from_MQbrain_618_r | 96.31 |
| HSbrain_342_l | 96.28 |
| Hsd_from_Mmd_519_r | 95.12 |
| HS_from_MQbrain_557_l | 94.76 |
| HSbrain_194_r | 94.74 |
| HS_from_MQbrain_1649_l | 94.72 |
| HS_from_MQbrain_1816_r | 94.61 |
| HS_from_MQbrain_57_l | 94.28 |
| HS_from_MM_321_r | 92.98 |
| Hsd_111_r | 92.66 |
| Hsd_from_Mmd_235_r | 92.57 |
| HSbrain_778_l | 91.71 |
| Hsd_from_Mmd_526_r | 89.64 |
| HS_from_MQbrain_1749_r | 89.49 |
| Hsd_from_Mmd_482_r | 88.64 |
| HS_from_MM_182_r | 88.50 |
| HS_from_MQbrain_346_r | 88.45 |
| HS_from_MM_161_l | 88.26 |
| HS_from_MQbrain_249_l | 88.26 |
| Hsd_82_l | 88.10 |
| HS_from_PT_136_l | 88.09 |
| HS_from_MQbrain_1871_r | 86.87 |
| HSbrain_756_l | 86.23 |
| HS_from_MQbrain_1730_r | 86.13 |
| HS_from_MQbrain_1751_r | 85.80 |
| HS_from_MQbrain_1566_l | 85.25 |
| Hsd_from_Mmd_412_l | 84.96 |
| Hsd_from_Mmd_515_l | 84.90 |
| HSbrain_2_l | 84.89 |
| HS_from_MM_155_r | 84.60 |
| HS_from_MQbrain_155_r | 84.50 |
| HS_from_MQbrain_1304_r | 84.49 |
| HS_from_MQbrain_81_r | 84.46 |
| HS_from_MQbrain_622_r | 83.61 |
| HS_from_MQbrain_1131_l | 83.55 |
| Hsd_56_l | 83.20 |
| Hsd_8_l | 83.04 |
| HS_212_r | 82.75 |
| HSbrain_212_l | 82.21 |
| HS_from_MQbrain_398_r | 82.13 |
| HS_from_MQbrain_1779_l | 81.98 |
| HS_from_MQbrain_1852_r | 81.57 |
| HSbrain_801_r | 81.51 |
| Hsd_from_Mmd_466_l | 80.93 |
| HS_from_MM_199_l | 79.91 |
| HS_from_MQbrain_1209_r | 79.23 |
| HSbrain_526_r | 78.78 |
| HS_from_MQbrain_151_r | 78.11 |
| HS_from_PT_103_l | 77.68 |
| HSbrain_113_r | 77.28 |
| Hsd_from_Mmd_271_r | 77.04 |
| Hsd_from_Mmd_172_l | 76.79 |
| HS_from_PT_180_l | 75.89 |
| HSbrain_697_l | 75.83 |
| Hsd_from_Mmd_438_r | 75.54 |
| HS_from_MQbrain_517_r | 75.18 |
| HS_from_MQbrain_1182_l | 75.16 |
| HS_from_MQbrain_160_l | 74.92 |
| HS_from_MQbrain_674_r | 74.90 |
| HS_282_l | 74.89 |
| HS_from_MQbrain_1002_l | 74.73 |
| HS_from_MQbrain_1740_l | 74.72 |
| HS_from_PT_340_l | 74.70 |
| HS_from_MQbrain_58_r | 74.41 |
| HS_from_MQbrain_1638_l | 74.01 |
| HS_174_l | 73.96 |
| Hsd_from_Mmd_228_r | 73.75 |
| HS_from_MQbrain_1325_r | 73.72 |
| Hsd_from_Mmd_186_l | 73.39 |
| HS_from_MQbrain_1788_r | 73.00 |
| HS_from_MM_12_l | 72.97 |
| HS_from_MQbrain_525_r | 72.69 |
| HS_from_MQbrain_1145_r | 72.63 |
| HS_from_MM_334_l | 71.92 |
| HS_from_MQbrain_1417_r | 71.89 |
| HS_from_MQbrain_370_r | 71.88 |
| HS_from_MQbrain_1692_l | 71.86 |
| HS_from_MQbrain_1363_r | 71.66 |
| Hsd_from_Mmd_96_l | 71.59 |
| HS_from_MQbrain_500_l | 71.53 |
| HS_from_MQbrain_949_l | 71.15 |
| HS_from_MQbrain_1297_l | 70.63 |
| Hsd_from_Mmd_461_l | 70.50 |
| HS_from_PT_248_l | 70.46 |
| HSbrain_585_l | 70.01 |
| HSbrain_435_r | 69.54 |
| HS_from_MM_316_r | 69.22 |
| HS_from_MQbrain_15_l | 69.02 |
| HS_from_PT_74_r | 68.99 |
| HS_from_MQbrain_1027_r | 68.90 |
| Hsd_from_Mmd_190_l | 68.83 |
| HS_from_MQbrain_1685_l | 68.54 |
| HSbrain_148_r | 68.52 |
| HS_from_MQbrain_714_l | 68.40 |
| HS_from_MQbrain_1886_r | 68.24 |
| HSbrain_71_l | 67.99 |
| HSbrain_351_l | 67.80 |
| HS_184_l | 67.16 |
| HS_from_MQbrain_1445_l | 67.15 |
| HSbrain_434_l | 67.13 |
| HSbrain_253_l | 67.08 |
| HS_68_l | 66.74 |
| HSbrain_631_r | 66.51 |
| HS_203_r | 66.49 |
| HS_from_MQbrain_1422_r | 66.39 |
| HS_from_MQbrain_1207_l | 66.20 |
| Hsd_from_Mmd_430_r | 65.51 |
| HSbrain_705_r | 64.83 |
| Hsd_from_Mmd_176_l | 64.49 |
| HS_from_MQbrain_1478_l | 63.78 |
| HS_216_r | 63.62 |
| HS_from_MQbrain_1640_r | 63.45 |
| HSbrain_289_l | 63.38 |
| HS_from_MQbrain_1835_r | 63.36 |
| HS_from_MM_301_l | 63.22 |
| HS_from_MM_40_l | 63.15 |
| HS_from_MQbrain_575_r | 63.09 |
| Hsd_from_Mmd_161_l | 62.81 |
| HS_from_MQbrain_113_l | 62.81 |
| HS_from_MM_336_r | 62.68 |
| HSbrain_77_r | 62.61 |
| HSbrain_1436_l | 62.57 |
| HS_from_MQbrain_100_l | 62.37 |
| HS_from_MQbrain_979_r | 62.19 |
| HS_from_MQbrain_31_r | 61.82 |
| HSbrain_116_l | 61.35 |
| HSbrain_557_r | 60.82 |
| HSbrain_157_l | 60.78 |
| HS_from_MQbrain_740_l | 60.73 |
| HSbrain_760_l | 60.42 |
| HSbrain_304_l | 60.38 |
| HS_from_PT_176_r | 60.13 |
| HS_from_MM_96_l | 59.61 |
| HS_from_MQbrain_69_r | 59.60 |
| HSbrain_376_r | 59.58 |
| HSbrain_630_l | 59.49 |
| HSbrain_282_l | 59.48 |
| HS_from_MQbrain_372_l | 59.38 |
| Hsd_from_Mmd_169_r | 59.27 |
| Hsd_59_r | 59.20 |
| HS_from_PT_206_l | 58.76 |
| HS_from_MQbrain_1_l | 58.07 |
| HS_180_r | 57.76 |
| HSbrain_375_r | 57.73 |
| HS_20_r | 56.85 |
| HSbrain_505_l | 56.76 |
| HSbrain_245_r | 56.71 |
| HS_from_MQbrain_132_l | 55.92 |
| HS_from_MQbrain_222_l | 55.76 |
| HSbrain_916_l | 55.74 |
| HS_106_l | 55.68 |
| HSbrain_739_l | 55.15 |
| HSbrain_223_r | 55.06 |
| HS_from_MM_38_l | 55.05 |
| HS_from_MQbrain_710_l | 55.00 |
| HSbrain_589_r | 54.91 |
| HS_from_MQbrain_484_r | 54.66 |
| HS_from_MQbrain_977_r | 54.29 |
| HS_from_MQbrain_802_l | 54.04 |
| HS_from_MQbrain_391_r | 53.80 |
| HS_from_MQbrain_240_l | 53.57 |
| HSbrain_106_r | 53.44 |
| HS_from_MQbrain_255_l | 53.09 |
| HS_from_MQbrain_1517_l | 52.95 |
| HS_179_l | 52.66 |
| HS_from_MQbrain_118_l | 52.25 |
| Hsd_from_Mmd_204_l | 52.22 |
| HS_215_l | 51.87 |
| HSbrain_195_l | 51.67 |
| HSbrain_424_l | 51.64 |
| HSbrain_448_l | 51.31 |
| HS_from_MM_126_l | 51.22 |
| Hsd_102_l | 51.20 |
| HSbrain_341_r | 50.96 |
| HS_from_MQbrain_235_l | 50.81 |
| HS_from_MQbrain_619_r | 50.44 |
| HS_from_MM_335_r | 50.36 |
| HSbrain_197_l | 49.79 |
| HS_from_MQbrain_869_l | 49.56 |
| HS_from_MQbrain_433_l | 49.52 |
| HS_10_l | 49.43 |
| HS_from_MQbrain_1520_r | 49.34 |
| HS_from_MQbrain_20_r | 49.05 |
| HS_from_MQbrain_1400_l | 48.14 |
| HSbrain_236_r | 48.09 |
| HS_from_MQbrain_713_r | 47.95 |
| HS_from_MQbrain_1654_l | 47.95 |
| HSbrain_14_r | 47.92 |
| HS_from_MQbrain_1765_r | 47.70 |
| HS_from_MQbrain_1672_r | 47.58 |
| HSbrain_206_l | 46.96 |
| HSbrain_209_r | 46.69 |
| HS_257_r | 46.69 |
| Hsd_from_Mmd_34_r | 46.61 |
| HS_286_l | 46.37 |
| HS_from_MQbrain_1769_r | 46.33 |
| Hsd_68_l | 45.49 |
| HSbrain_262_l | 45.22 |
| HS_from_MQbrain_535_r | 45.21 |
| HS_from_MQbrain_468_l | 45.02 |
| HS_from_MM_159_l | 44.47 |
| Hsd_from_Mmd_507_l | 44.29 |
| HS_from_MQbrain_1527_l | 44.13 |
| HS_from_MQbrain_256_l | 44.09 |
| HS_from_PT_338_r | 44.05 |
| HS_from_MQbrain_894_l | 43.92 |
| HSbrain_158_l | 43.60 |
| HS_192_r | 43.57 |
| HS_from_MQbrain_1739_r | 43.35 |
| HSbrain_42_r | 43.31 |
| HS_from_MQbrain_466_r | 43.22 |
| HS_from_MQbrain_45_r | 42.99 |
| HS_from_MQbrain_890_r | 42.96 |
| HS_from_MQbrain_997_l | 42.80 |
| HS_from_MQbrain_135_l | 42.75 |
| Hsd_from_Mmd_81_l | 42.55 |
| HS_from_MQbrain_832_l | 42.51 |
| HSbrain_764_r | 42.39 |
| HS_from_PT_108_l | 42.07 |
| Hsd_from_Mmd_324_r | 41.99 |
| HS_from_MQbrain_632_l | 41.42 |
| HS_from_MQbrain_110_r | 41.30 |
| Hsd_from_Mmd_478_l | 40.92 |
| HS_from_MQbrain_1757_l | 40.65 |
| HS_from_MQbrain_1323_r | 40.38 |
| HS_from_MQbrain_1497_r | 40.38 |
| HS_from_MQbrain_186_r | 40.23 |
| HS_from_MM_309_r | 39.96 |
| HS_from_MQbrain_17_r | 39.73 |
| Hsd_from_Mmd_183_r | 39.66 |
| Hsd_86_l | 39.30 |
| HSbrain_235_r | 39.24 |
| HS_from_MQbrain_545_l | 39.21 |
| HS_from_MQbrain_1332_r | 39.17 |
| HSbrain_601_r | 39.07 |
| HS_from_MQbrain_192_l | 38.87 |
| HS_from_MQbrain_1592_l | 38.47 |
| HSbrain_418_l | 38.33 |
| HSbrain_24_l | 38.27 |
| HS_from_MM_292_r | 37.98 |
| HS_from_MQbrain_1064_r | 37.45 |
| HS_from_MM_227_l | 37.41 |
| HS_from_MQbrain_1716_r | 37.37 |
| HS_from_MQbrain_1801_r | 37.34 |
| HS_from_MQbrain_1724_l | 37.26 |
| HSbrain_263_r | 37.25 |
| Hsd_from_Mmd_399_l | 37.23 |
| HS_85_l | 37.14 |
| Hsd_37_l | 37.10 |
| HS_from_MQbrain_1100_l | 37.05 |
| HS_from_MQbrain_502_r | 36.99 |
| HS_from_MQbrain_1528_r | 36.65 |
| HS_from_MQbrain_722_r | 36.55 |
| HS_from_MQbrain_1700_l | 36.53 |
| HSbrain_482_l | 36.52 |
| HS_from_MM_81_l | 36.43 |
| HS_from_MQbrain_21_l | 36.19 |
| HS_from_MQbrain_322_r | 35.61 |
| HS_from_MQbrain_1350_r | 35.60 |
| HS_from_MQbrain_80_l | 35.53 |
| HS_36_l | 35.10 |
| HS_from_MQbrain_664_l | 35.08 |
| HSbrain_581_r | 35.02 |
| HS_81_l | 34.96 |
| HSbrain_91_l | 34.91 |
| HSbrain_397_r | 34.86 |
| HS_231_r | 34.84 |
| HS_from_MM_303_l | 34.63 |
| HS_from_MQbrain_461_r | 34.59 |
| HSbrain_39_r | 34.48 |
| HS_217_l | 34.38 |
| Hsd_from_Mmd_92_r | 34.05 |
| HS_from_MQbrain_1128_l | 33.94 |
| HS_from_MQbrain_1203_l | 33.86 |
| HS_from_MM_349_l | 33.83 |
| HS_from_MQbrain_1462_l | 33.63 |
| HSbrain_90_l | 33.58 |
| HSbrain_131_l | 33.57 |
| HSbrain_605_r | 33.48 |
| HSbrain_483_l | 33.11 |
| HS_46_r | 33.07 |
| HSbrain_1174_l | 32.55 |
| HS_from_MQbrain_412_r | 32.20 |
| HS_from_MQbrain_1235_r | 32.06 |
| HS_from_MQbrain_385_r | 31.99 |
| Hsd_from_Mmd_383_l | 31.91 |
| Hsd_from_Mmd_229_l | 31.88 |
| HSbrain_517_l | 31.74 |
| HSbrain_730_r | 31.64 |
| HS_from_MQbrain_792_l | 31.62 |
| HS_from_MQbrain_1048_r | 31.28 |
| Hsd_43_l | 31.19 |
| HS_from_MQbrain_555_l | 31.14 |
| HSbrain_642_r | 31.13 |
| Hsd_from_Mmd_42_r | 30.99 |
| HS_from_MQbrain_1661_l | 30.77 |
| HS_from_MQbrain_799_l | 30.71 |
| HSbrain_285_l | 30.64 |
| HS_from_MQbrain_582_r | 30.61 |
| HS_from_MQbrain_656_l | 30.46 |
| HS_239_r | 30.45 |
| HSbrain_737_r | 30.44 |
| HS_from_MQbrain_1092_l | 30.44 |
| HS_300_l | 30.41 |
| HS_from_MQbrain_353_r | 30.38 |
| HSbrain_433_l | 30.34 |
| HS_from_MQbrain_1383_r | 30.08 |
| HS_from_MQbrain_952_l | 30.02 |
| HS_from_MQbrain_924_l | 29.92 |
| HS_from_PT_217_r | 29.85 |
| HS_from_MQbrain_1851_r | 29.83 |
| HS_from_MQbrain_881_l | 29.79 |
| HS_from_MQbrain_530_l | 29.55 |
| HS_from_MQbrain_1481_r | 29.54 |
| HS_299_l | 29.47 |
| HS_from_MQbrain_79_r | 29.36 |
| HSbrain_562_r | 29.36 |
| Hsd_from_Mmd_354_l | 28.95 |
| HS_from_MQbrain_1858_r | 28.80 |
| HS_8_l | 28.77 |
| Hsd_from_Mmd_481_l | 28.77 |
| HS_from_PT_139_l | 28.77 |
| HS_71_l | 28.70 |
| HS_from_MQbrain_355_r | 28.65 |
| HS_from_MQbrain_1879_r | 28.46 |
| HS_from_PT_310_l | 28.33 |
| HS_from_MQbrain_1602_r | 28.22 |
| HSbrain_412_l | 28.22 |
| HS_from_MM_49_l | 28.19 |
| HS_from_MQbrain_800_l | 28.04 |
| HSbrain_380_r | 28.02 |
| HSbrain_941_l | 27.88 |
| HSbrain_241_l | 27.85 |
| Hsd_67_r | 27.78 |
| HSbrain_627_r | 27.61 |
| HS_250_l | 27.55 |
| HS_from_MQbrain_1169_l | 27.22 |
| HS_from_PT_73_r | 27.20 |
| HS_from_MM_203_l | 27.16 |
| HS_from_MQbrain_573_l | 27.14 |
| HS_from_MQbrain_309_l | 26.75 |
| HSbrain_453_r | 26.71 |
| HSbrain_461_l | 26.59 |
| HS_from_MQbrain_1734_l | 26.58 |
| HS_from_MQbrain_744_l | 26.48 |
| HS_from_MQbrain_382_r | 26.42 |
| HS_from_MQbrain_1420_r | 26.36 |
| HS_from_MQbrain_834_r | 26.24 |
| Hsd_from_Mmd_174_r | 26.19 |
| Hsd_from_Mmd_44_l | 26.07 |
| Hsd_from_Mmd_69_l | 26.04 |
| HS_from_MQbrain_244_l | 26.02 |
| HS_from_MQbrain_64_r | 25.96 |
| HS_from_MM_13_r | 25.89 |
| HS_from_MQbrain_1116_l | 25.82 |
| HS_from_MM_260_r | 25.78 |
| HS_from_MQbrain_1168_l | 25.63 |
| HSbrain_790_r | 25.61 |
| HS_from_MQbrain_687_r | 25.58 |
| HSbrain_4_r | 25.57 |
| HSbrain_224_r | 25.38 |
| HS_from_MM_198_l | 25.27 |
| HS_from_PT_38_r | 25.14 |
| HSbrain_917_l | 24.97 |
| HSbrain_748_r | 24.89 |
| Hsd_55_r | 24.87 |
| HS_from_MQbrain_26_l | 24.76 |
| HSbrain_52_r | 24.74 |
| HS_from_MQbrain_96_r | 24.65 |
| HS_from_MQbrain_513_l | 24.59 |
| HS_from_MQbrain_273_l | 24.56 |
| Hsd_24_l | 24.38 |
| HSbrain_1419_l | 24.37 |
| HS_from_MQbrain_224_l | 24.13 |
| Hsd_44_l | 23.85 |
| HS_from_PT_251_l | 23.73 |
| HSbrain_28_r | 23.68 |
| HS_from_MQbrain_1658_r | 23.47 |
| HS_147_l | 23.40 |
| HS_from_MM_191_l | 23.35 |
| HSbrain_128_r | 23.27 |
| HS_from_MM_338_l | 23.22 |
| HS_from_PT_195_l | 23.16 |
| HSbrain_422_l | 23.11 |
| HS_from_MQbrain_1746_l | 23.03 |
| HS_from_MQbrain_808_l | 23.00 |
| HS_from_MQbrain_585_r | 22.89 |
| HSbrain_239_l | 22.71 |
| HSbrain_1410_l | 22.68 |
| Hsd_from_Mmd_428_r | 22.45 |
| HSbrain_727_l | 22.41 |
| HS_from_MQbrain_1097_r | 22.37 |
| HS_from_MQbrain_1725_r | 22.32 |
| HS_254_r | 22.32 |
| HS_from_MQbrain_986_l | 22.31 |
| HS_297_r | 22.18 |
| HSbrain_788_r | 22.17 |
| Hsd_from_Mmd_222_r | 22.16 |
| HS_306_l | 22.09 |
| HS_from_MQbrain_809_l | 21.62 |
| HSbrain_531_r | 21.58 |
| HSbrain_657_l | 21.56 |
| HSbrain_787_r | 21.53 |
| Hsd_from_Mmd_157_l | 21.46 |
| HS_from_MQbrain_1612_r | 21.46 |
| HS_from_MQbrain_1540_l | 21.33 |
| HS_from_MQbrain_37_l | 21.12 |
| Hsd_from_Mmd_429_l | 21.12 |
| HSbrain_208_r | 20.78 |
| HSbrain_211_l | 20.62 |
| HS_from_MQbrain_628_l | 20.61 |
| HSbrain_444_l | 20.61 |
| HS_from_MQbrain_838_l | 20.56 |
| HS_from_PT_193_r | 20.52 |
| HS_from_MM_87_l | 20.41 |
| HSbrain_777_l | 20.39 |
| HS_from_MQbrain_295_l | 20.33 |
| Hsd_from_Mmd_340_r | 20.26 |
| HSbrain_486_r | 20.19 |
| HSbrain_222_l | 20.17 |
| HS_from_MQbrain_564_r | 20.10 |
| HS_295_r | 20.06 |
| HS_from_MQbrain_228_r | 20.00 |
| HS_from_MQbrain_625_l | 19.94 |
| HS_from_PT_7_r | 19.85 |
| HS_from_MQbrain_506_l | 19.79 |
| HS_from_MQbrain_483_r | 19.69 |
| HS_from_MQbrain_1314_r | 19.69 |
| HSbrain_324_l | 19.66 |
| HS_from_MQbrain_1595_l | 19.52 |
| HS_from_MM_14_r | 19.51 |
| HS_from_MQbrain_2670_r | 19.49 |
| HSbrain_782_r | 19.39 |
| HS_from_PT_313_l | 19.26 |
| HSbrain_379_r | 19.23 |
| Hsd_from_Mmd_211_r | 19.19 |
| HS_177_r | 19.18 |
| HS_59_l | 19.16 |
| HS_from_MQbrain_1208_l | 19.05 |
| HS_80_l | 19.03 |
| HS_from_MQbrain_1556_l | 18.88 |
| HS_272_r | 18.72 |
| HS_from_MQbrain_874_r | 18.69 |
| HS_from_MQbrain_143_r | 18.66 |
| HS_from_MQbrain_1109_l | 18.59 |
| HS_from_MM_184_l | 18.57 |
| HS_from_MQbrain_1290_l | 18.49 |
| HSbrain_577_r | 18.45 |
| HSbrain_337_l | 18.24 |
| HSbrain_97_l | 18.15 |
| HS_41_r | 18.13 |
| HS_from_MQbrain_760_r | 18.00 |
| HSbrain_556_r | 17.96 |
| HSbrain_1384_l | 17.96 |
| HS_187_r | 17.93 |
| HS_from_PT_122_r | 17.92 |
| HSbrain_747_l | 17.76 |
| HS_from_MQbrain_1087_r | 17.69 |
| HS_from_MQbrain_870_l | 17.66 |
| HS_from_PT_156_r | 17.38 |
| HS_from_MQbrain_1147_r | 17.05 |
| Hsd_13_l | 16.95 |
| HSbrain_489_l | 16.94 |
| Hsd_from_Mmd_85_r | 16.92 |
| HS_from_MQbrain_194_l | 16.83 |
| HS_294_r | 16.74 |
| HS_from_PT_320_l | 16.59 |
| HSbrain_647_r | 16.58 |
| HSbrain_161_r | 16.57 |
| HS_from_MQbrain_578_r | 16.56 |
| HS_from_MQbrain_25_r | 16.56 |
| HSbrain_763_r | 16.39 |
| HS_from_MQbrain_848_r | 16.38 |
| HS_from_PT_272_r | 16.35 |
| HS_from_MM_287_l | 16.33 |
| HS_153_r | 16.26 |
| HS_from_MM_307_l | 16.22 |
| HSbrain_112_l | 16.20 |
| HS_from_MQbrain_1439_l | 16.18 |
| HS_from_MQbrain_899_l | 16.17 |
| Hsd_89_r | 16.14 |
| HS_from_MQbrain_1726_r | 16.13 |
| HSbrain_230_r | 16.04 |
| HSbrain_1412_l | 15.97 |
| HS_from_MQbrain_34_r | 15.96 |
| HS_from_MQbrain_1571_r | 15.79 |
| HS_from_MQbrain_339_l | 15.61 |
| HS_from_MQbrain_137_r | 15.61 |
| HSbrain_173_r | 15.56 |
| HSbrain_389_l | 15.54 |
| HS_from_MQbrain_1120_r | 15.49 |
| HSbrain_40_r | 15.41 |
| HS_from_MQbrain_253_l | 15.37 |
| HS_from_PT_162_r | 15.37 |
| HSbrain_62_l | 15.34 |
| Hsd_6_r | 15.26 |
| HS_from_MQbrain_437_l | 15.23 |
| HS_from_MQbrain_366_r | 15.16 |
| HS_from_PT_22_l | 15.15 |
| HSbrain_297_r | 14.96 |
| HS_from_MQbrain_24_r | 14.95 |
| HS_from_MQbrain_1322_l | 14.89 |
| HS_from_MQbrain_842_r | 14.88 |
| HS_from_MM_89_r | 14.86 |
| HS_from_MQbrain_676_r | 14.81 |
| HS_from_MQbrain_1264_r | 14.74 |
| Hsd_from_Mmd_90_r | 14.74 |
| HS_from_PT_292_l | 14.70 |
| HSbrain_563_l | 14.57 |
| Hsd_from_Mmd_80_r | 14.56 |
| HS_from_MQbrain_1354_l | 14.56 |
| Hsd_from_Mmd_95_l | 14.50 |
| HS_from_MQbrain_1809_r | 14.47 |
| HSbrain_458_l | 14.44 |
| HS_from_MQbrain_756_l | 14.40 |
| HSbrain_59_l | 14.34 |
| HS_from_MQbrain_1163_l | 14.31 |
| HS_from_PT_140_r | 14.24 |
| HS_from_MQbrain_1892_r | 14.19 |
| HS_from_MQbrain_354_r | 14.17 |
| HSbrain_661_r | 14.15 |
| Hsd_from_Mmd_389_r | 14.14 |
| HS_from_MM_90_r | 14.13 |
| Hsd_27_l | 14.13 |
| HS_213_r | 14.10 |
| HS_from_MQbrain_435_l | 14.03 |
| HS_from_MQbrain_1457_l | 14.03 |
| HS_from_MQbrain_1560_l | 14.01 |
| HS_from_MQbrain_641_r | 14.00 |
| HS_from_MQbrain_1659_r | 13.85 |
| HS_from_MM_120_l | 13.78 |
| Hsd_from_Mmd_367_l | 13.74 |
| HS_from_MM_226_r | 13.53 |
| HSbrain_84_l | 13.53 |
| HS_from_MQbrain_1600_r | 13.50 |
| HS_from_MQbrain_444_l | 13.49 |
| HSbrain_572_r | 13.46 |
| HSbrain_660_l | 13.33 |
| HS_from_MQbrain_296_r | 13.28 |
| HS_from_PT_187_l | 13.27 |
| HS_from_MQbrain_701_l | 13.24 |
| Hsd_from_Mmd_156_r | 13.22 |
| HS_from_MQbrain_1361_l | 13.21 |
| HS_from_MQbrain_215_l | 13.18 |
| HS_from_PT_4_r | 13.03 |
| HS_from_MM_192_r | 12.92 |
| HS_from_MQbrain_1095_r | 12.76 |
| HS_from_MQbrain_1343_r | 12.74 |
| HSbrain_271_r | 12.71 |
| HSbrain_137_l | 12.68 |
| HSbrain_78_l | 12.54 |
| Hsd_from_Mmd_195_r | 12.40 |
| HS_from_MQbrain_759_r | 12.38 |
| Hsd_93_l | 12.36 |
| HSbrain_272_r | 12.32 |
| HSbrain_187_l | 12.32 |
| HS_from_MQbrain_300_r | 12.29 |
| HS_from_MQbrain_72_l | 12.28 |
| HS_from_MQbrain_872_l | 12.26 |
| HS_from_MQbrain_804_l | 12.23 |
| HSbrain_553_l | 12.16 |
| Hsd_60_l | 12.04 |
| Hsd_84_l | 11.97 |
| HS_from_MQbrain_820_l | 11.96 |
| HS_from_MQbrain_811_r | 11.96 |
| HS_from_MQbrain_1206_r | 11.96 |
| HS_from_MQbrain_1424_r | 11.94 |
| HS_from_PT_254_r | 11.89 |
| HS_from_PT_61_r | 11.87 |
| HS_from_MQbrain_613_r | 11.86 |
| HSbrain_721_l | 11.70 |
| HS_from_MQbrain_1217_l | 11.69 |
| Hsd_from_Mmd_527_r | 11.56 |
| HS_from_MM_98_r | 11.53 |
| HS_from_MQbrain_1665_l | 11.43 |
| Hsd_from_Mmd_182_l | 11.41 |
| Hsd_from_Mmd_296_r | 11.39 |
| HS_90_l | 11.39 |
| HS_from_MQbrain_796_r | 11.36 |
| HS_from_MQbrain_537_l | 11.34 |
| HS_from_MQbrain_1201_l | 11.33 |
| HS_from_MQbrain_620_l | 11.23 |
| HS_from_MQbrain_967_r | 11.18 |
| HS_from_MQbrain_1292_r | 11.08 |
| HS_from_PT_66_r | 11.07 |
| HS_from_MM_158_l | 11.06 |
| HS_from_MQbrain_112_l | 11.05 |
| HS_from_MQbrain_1513_r | 11.02 |
| HS_from_MQbrain_1690_l | 10.98 |
| Hsd_from_Mmd_226_r | 10.95 |
| HS_from_MQbrain_839_r | 10.92 |
| HS_from_MM_187_l | 10.90 |
| HS_from_MQbrain_148_l | 10.90 |
| Hsd_45_r | 10.80 |
| HS_from_MQbrain_1216_l | 10.80 |
| HS_from_PT_196_r | 10.79 |
| HS_from_MQbrain_1502_l | 10.78 |
| Hsd_from_Mmd_372_l | 10.75 |
| HSbrain_378_l | 10.71 |
| HSbrain_82_r | 10.70 |
| HS_from_MQbrain_778_r | 10.70 |
| HS_from_MQbrain_1334_r | 10.69 |
| HS_from_MQbrain_1657_r | 10.62 |
| Hsd_from_Mmd_275_r | 10.60 |
| HSbrain_663_r | 10.59 |
| HSbrain_491_r | 10.47 |
| HS_from_MQbrain_1214_l | 10.46 |
| HS_from_MQbrain_813_l | 10.45 |
| HS_from_MQbrain_771_r | 10.40 |
| HS_from_MQbrain_1958_r | 10.36 |
| HSbrain_695_l | 10.34 |
| HSbrain_789_r | 10.34 |
| HSbrain_479_l | 10.28 |
| HS_from_MQbrain_471_r | 10.24 |
| HS_from_MQbrain_1985_r | 10.17 |
| HSbrain_529_r | 10.10 |
| HSbrain_357_r | 10.10 |
| Hsd_from_Mmd_532_l | 10.04 |
| Hsd_from_Mmd_94_l | 10.00 |
| HS_from_MQbrain_1243_r | 9.90 |
| HSbrain_734_r | 9.87 |
| HS_from_PT_332_l | 9.85 |
| HS_from_MQbrain_1396_r | 9.81 |
| HS_67_l | 9.78 |
| HS_260_l | 9.76 |
| HSbrain_177_r | 9.75 |
| HSbrain_707_r | 9.72 |
| Hsd_from_Mmd_435_l | 9.69 |
| HSbrain_135_r | 9.63 |
| HS_63_l | 9.59 |
| HS_from_MQbrain_1570_r | 9.52 |
| HS_161_r | 9.51 |
| HSbrain_370_r | 9.51 |
| HSbrain_9_l | 9.49 |
| Hsd_from_Mmd_326_l | 9.49 |
| HSbrain_83_r | 9.49 |
| HS_from_PT_40_l | 9.49 |
| Hsd_47_l | 9.46 |
| HS_from_MQbrain_864_l | 9.43 |
| HS_from_MQbrain_1760_r | 9.40 |
| HS_from_MQbrain_1037_l | 9.33 |
| HSbrain_711_r | 9.31 |
| HS_from_MQbrain_1531_r | 9.27 |
| HS_from_MQbrain_1139_l | 9.26 |
| HSbrain_506_l | 9.24 |
| HS_from_MQbrain_1404_l | 9.22 |
| HS_from_MQbrain_1317_l | 9.20 |
| HS_137_r | 9.18 |
| HS_from_MQbrain_68_l | 9.14 |
| Hsd_from_Mmd_259_l | 9.11 |
| HS_from_MQbrain_1378_l | 9.06 |
| HS_from_MQbrain_170_r | 9.03 |
| HSbrain_702_l | 8.99 |
| HS_from_MQbrain_1466_r | 8.96 |
| HS_from_MM_35_l | 8.96 |
| HS_from_MQbrain_117_l | 8.94 |
| HSbrain_5_l | 8.93 |
| HS_from_MQbrain_476_r | 8.92 |
| HS_from_MQbrain_579_r | 8.91 |
| HS_from_MM_294_l | 8.85 |
| HS_from_MQbrain_509_r | 8.85 |
| HS_from_MQbrain_1516_l | 8.83 |
| HS_from_MM_122_r | 8.80 |
| HSbrain_180_r | 8.79 |
| HS_from_MQbrain_1628_l | 8.78 |
| HS_from_MQbrain_930_r | 8.71 |
| HSbrain_274_r | 8.70 |
| HSbrain_356_r | 8.69 |
| HS_from_PT_123_r | 8.69 |
| Hsd_98_r | 8.68 |
| HS_100_l | 8.54 |
| HS_from_MQbrain_478_r | 8.53 |
| HS_from_PT_191_r | 8.52 |
| HS_from_MQbrain_1772_r | 8.51 |
| HS_183_l | 8.51 |
| HS_from_MQbrain_836_l | 8.49 |
| Hsd_from_Mmd_219_l | 8.42 |
| HSbrain_1337_r | 8.35 |
| HS_43_r | 8.34 |
| HS_62_l | 8.32 |
| HS_from_MM_193_r | 8.32 |
| HSbrain_806_l | 8.24 |
| HS_from_MM_135_r | 8.21 |
| HS_from_MQbrain_1487_r | 8.21 |
| HS_from_MQbrain_1494_r | 8.20 |
| HS_from_MM_300_l | 8.17 |
| HS_from_MQbrain_1410_l | 8.14 |
| HS_from_MQbrain_1805_r | 8.13 |
| HS_from_MM_20_r | 8.13 |
| HSbrain_499_r | 8.10 |
| HS_from_MQbrain_16_l | 7.96 |
| HSbrain_258_r | 7.95 |
| HSbrain_198_l | 7.92 |
| HS_from_MQbrain_1884_r | 7.89 |
| HSbrain_646_l | 7.88 |
| HSbrain_325_l | 7.86 |
| HSbrain_254_r | 7.85 |
| HS_from_MQbrain_954_r | 7.84 |
| HS_from_MQbrain_1072_r | 7.84 |
| Hsd_from_Mmd_413_l | 7.78 |
| HS_from_MQbrain_212_r | 7.74 |
| HS_from_MQbrain_1522_r | 7.73 |
| HS_from_MM_172_r | 7.69 |
| HS_from_MQbrain_920_r | 7.68 |
| HS_from_MQbrain_923_l | 7.68 |
| HS_from_MM_123_l | 7.67 |
| HS_from_MM_165_l | 7.64 |
| Hsd_7_r | 7.55 |
| HSbrain_609_r | 7.53 |
| Hsd_from_Mmd_35_l | 7.49 |
| HS_from_MQbrain_1414_r | 7.45 |
| HS_from_MQbrain_601_l | 7.43 |
| HS_from_MM_242_l | 7.39 |
| HS_from_MQbrain_634_l | 7.38 |
| HS_from_PT_315_l | 7.36 |
| HS_from_PT_135_l | 7.35 |
| HSbrain_512_r | 7.28 |
| Hsd_from_Mmd_45_r | 7.23 |
| HS_from_PT_182_r | 7.19 |
| HS_from_PT_168_r | 7.14 |
| HS_from_MQbrain_552_r | 7.13 |
| HSbrain_13_l | 7.13 |
| HS_from_MM_317_l | 7.12 |
| Hsd_from_Mmd_492_l | 7.11 |
| HS_from_MQbrain_1662_l | 7.08 |
| HSbrain_332_l | 7.07 |
| HS_from_MQbrain_572_l | 7.03 |
| HS_from_MQbrain_1894_l | 7.00 |
| HS_from_MM_313_r | 7.00 |
| HSbrain_140_l | 6.96 |
| HSbrain_654_l | 6.95 |
| HSbrain_352_l | 6.94 |
| HS_from_MQbrain_934_r | 6.92 |
| HS_96_r | 6.92 |
| HS_from_MQbrain_1362_r | 6.91 |
| Hsd_26_l | 6.89 |
| HSbrain_58_l | 6.87 |
| HS_from_MQbrain_1676_r | 6.87 |
| Hsd_from_Mmd_499_l | 6.85 |
| HS_from_MQbrain_946_l | 6.84 |
| HSbrain_333_l | 6.83 |
| HS_11_l | 6.83 |
| HS_from_PT_205_r | 6.81 |
| HS_from_MQbrain_1674_l | 6.80 |
| Hsd_from_Mmd_68_r | 6.78 |
| HS_from_MQbrain_1248_r | 6.76 |
| HSbrain_12_l | 6.74 |
| HS_158_r | 6.72 |
| HSbrain_633_r | 6.71 |
| HS_from_MQbrain_420_r | 6.70 |
| HSbrain_16_l | 6.68 |
| Hsd_35_r | 6.64 |
| HS_from_MM_331_l | 6.61 |
| HS_271_l | 6.60 |
| HS_from_MQbrain_984_l | 6.60 |
| HS_210_r | 6.59 |
| HSbrain_561_r | 6.50 |
| HS_from_MQbrain_717_r | 6.48 |
| HS_from_MQbrain_1180_r | 6.40 |
| HS_from_MQbrain_1268_l | 6.35 |
| HS_from_MQbrain_1312_l | 6.30 |
| HSbrain_374_l | 6.25 |
| HSbrain_443_r | 6.24 |
| HS_from_MQbrain_1089_r | 6.22 |
| HS_from_MQbrain_1646_r | 6.22 |
| HS_from_MQbrain_153_l | 6.19 |
| HS_from_MQbrain_1432_l | 6.17 |
| HS_from_MQbrain_1671_r | 6.17 |
| HS_from_MQbrain_241_r | 6.14 |
| Hsd_from_Mmd_197_r | 6.13 |
| HS_from_MQbrain_1782_r | 6.12 |
| HS_from_MQbrain_1823_r | 6.11 |
| HSbrain_407_l | 6.11 |
| HSbrain_736_r | 6.10 |
| HS_from_MM_164_r | 6.10 |
| HS_from_MQbrain_293_l | 6.07 |
| HS_298_r | 6.07 |
| HS_from_PT_216_r | 6.05 |
| HS_from_MQbrain_1883_r | 6.04 |
| HS_from_MQbrain_1080_l | 6.04 |
| HSbrain_670_l | 6.03 |
| HS_from_MQbrain_945_l | 6.02 |
| HS_from_MQbrain_402_r | 6.02 |
| HS_from_MQbrain_465_l | 5.99 |
| HS_from_MQbrain_549_l | 5.98 |
| HS_from_PT_218_r | 5.98 |
| HSbrain_519_l | 5.94 |
| HSbrain_811_r | 5.93 |
| HSbrain_497_l | 5.90 |
| HS_from_MQbrain_1485_r | 5.89 |
| HS_from_MM_118_l | 5.89 |
| HSbrain_471_l | 5.89 |
| HS_from_MQbrain_8_r | 5.89 |
| HSbrain_699_r | 5.89 |
| Hsd_from_Mmd_485_l | 5.88 |
| Hsd_from_Mmd_54_r | 5.85 |
| HS_from_MQbrain_596_r | 5.84 |
| HSbrain_429_r | 5.83 |
| Hsd_76_l | 5.81 |
| Hsd_from_Mmd_148_l | 5.76 |
| HS_from_MQbrain_569_r | 5.74 |
| HS_from_MQbrain_1642_r | 5.72 |
| HSbrain_1398_l | 5.70 |
| HSbrain_442_r | 5.68 |
| HSbrain_758_l | 5.61 |
| HSbrain_602_r | 5.61 |
| HSbrain_686_l | 5.59 |
| HSbrain_504_r | 5.59 |
| HSbrain_278_r | 5.57 |
| HS_from_MQbrain_1811_l | 5.55 |
| HS_from_MQbrain_866_l | 5.54 |
| HS_266_r | 5.54 |
| HSbrain_86_r | 5.53 |
| HSbrain_411_l | 5.53 |
| HS_from_MQbrain_1384_r | 5.51 |
| HS_from_MQbrain_1896_l | 5.46 |
| Hsd_51_r | 5.41 |
| HS_from_MQbrain_1326_r | 5.40 |
| HS_from_MQbrain_787_r | 5.40 |
| Hsd_from_Mmd_74_r | 5.38 |
| HS_270_l | 5.38 |
| HS_from_MQbrain_1056_l | 5.37 |
| HS_296_r | 5.37 |
| Hsd_from_Mmd_476_l | 5.36 |
| HSbrain_498_l | 5.35 |
| Hsd_40_r | 5.34 |
| HSbrain_232_l | 5.33 |
| HSbrain_119_l | 5.32 |
| HS_from_MQbrain_1474_l | 5.32 |
| HSbrain_1434_r | 5.32 |
| HS_from_MQbrain_1034_r | 5.31 |
| HS_from_MQbrain_817_l | 5.30 |
| HS_from_MM_124_r | 5.30 |
| HSbrain_1423_r | 5.23 |
| Hsd_from_Mmd_533_l | 5.22 |
| HS_from_MQbrain_1503_r | 5.21 |
| HS_from_MM_54_l | 5.20 |
| HS_from_MQbrain_1836_l | 5.19 |
| Hsd_from_Mmd_263_r | 5.17 |
| HSbrain_314_l | 5.16 |
| HS_from_PT_157_l | 5.16 |
| HS_from_MQbrain_777_r | 5.15 |
| HS_from_MQbrain_1606_r | 5.15 |
| HSbrain_466_l | 5.14 |
| HS_from_MM_194_r | 5.14 |
| HS_from_MQbrain_915_l | 5.12 |
| HSbrain_571_l | 5.09 |
| HS_from_MQbrain_1181_r | 5.09 |
| Hsd_from_Mmd_249_r | 5.05 |
| HSbrain_111_l | 5.04 |
| HSbrain_238_l | 5.04 |
| HSbrain_214_l | 5.03 |
| HS_293_r | 5.01 |
| Hsd_from_Mmd_343_l | 4.99 |
| HS_273_l | 4.98 |
| HS_from_MM_236_l | 4.96 |
| HSbrain_394_l | 4.95 |
| HS_from_MQbrain_1793_r | 4.95 |
| HS_from_MQbrain_812_r | 4.94 |
| HSbrain_339_l | 4.92 |
| HS_14_l | 4.90 |
| HS_252_l | 4.87 |
| HSbrain_1392_l | 4.86 |
| HS_103_r | 4.86 |
| HS_from_PT_1_r | 4.86 |
| HS_251_l | 4.82 |
| HSbrain_294_l | 4.82 |
| HS_from_MQbrain_907_r | 4.81 |
| HS_from_MM_359_r | 4.80 |
| HS_from_MQbrain_521_r | 4.77 |
| HSbrain_1388_l | 4.75 |
| Hsd_from_Mmd_436_r | 4.74 |
| HS_60_r | 4.73 |
| HSbrain_25_r | 4.72 |
| HSbrain_261_l | 4.72 |
| HS_from_PT_155_r | 4.70 |
| Hsd_from_Mmd_490_l | 4.66 |
| HS_from_MM_63_l | 4.64 |
| HSbrain_795_l | 4.64 |
| HSbrain_447_r | 4.64 |
| Hsd_from_Mmd_280_l | 4.61 |
| HSbrain_678_l | 4.61 |
| HSbrain_170_r | 4.60 |
| HSbrain_1391_l | 4.58 |
| HSbrain_1387_l | 4.56 |
| HS_from_MQbrain_43_r | 4.54 |
| Hsd_from_Mmd_437_r | 4.54 |
| HS_from_MQbrain_1762_l | 4.53 |
| HS_from_PT_221_r | 4.52 |
| HS_from_PT_288_l | 4.46 |
| HS_from_MQbrain_1377_l | 4.44 |
| HS_from_MM_149_r | 4.41 |
| HSbrain_674_l | 4.40 |
| Hsd_from_Mmd_286_r | 4.40 |
| HSbrain_46_r | 4.40 |
| Hsd_from_Mmd_262_l | 4.39 |
| HS_181_r | 4.32 |
| HSbrain_689_l | 4.31 |
| HSbrain_322_r | 4.31 |
| HSbrain_533_r | 4.30 |
| HSbrain_468_l | 4.28 |
| HS_from_MQbrain_1234_l | 4.28 |
| HS_from_MQbrain_1518_r | 4.26 |
| Hsd_from_Mmd_506_l | 4.24 |
| HSbrain_94_l | 4.24 |
| HSbrain_681_l | 4.24 |
| HS_from_MQbrain_1058_l | 4.22 |
| HS_from_MM_147_l | 4.22 |
| HS_from_MM_114_r | 4.22 |
| Hsd_from_Mmd_524_r | 4.21 |
| HS_from_MQbrain_1301_l | 4.17 |
| HSbrain_449_r | 4.16 |
| HS_from_MQbrain_1391_l | 4.14 |
| HS_from_MM_160_l | 4.14 |
| HSbrain_336_l | 4.14 |
| HS_from_MQbrain_168_l | 4.14 |
| HS_from_MQbrain_904_r | 4.09 |
| HSbrain_805_l | 4.08 |
| HSbrain_706_l | 4.08 |
| HSbrain_666_l | 4.08 |
| Hsd_from_Mmd_333_r | 4.01 |
| HSbrain_683_l | 4.00 |
| HS_from_MQbrain_542_l | 3.97 |
| HSbrain_1390_l | 3.97 |
| HSbrain_302_l | 3.96 |
| HS_132_l | 3.95 |
| HS_from_MM_178_r | 3.95 |
| HSbrain_377_r | 3.94 |
| HS_from_PT_35_r | 3.93 |
| HS_from_MQbrain_91_l | 3.92 |
| Hsd_from_Mmd_274_l | 3.91 |
| HS_from_MQbrain_361_l | 3.90 |
| HS_162_l | 3.89 |
| HSbrain_773_l | 3.88 |
| HS_from_MQbrain_835_r | 3.86 |
| HSbrain_81_l | 3.85 |
| HS_from_MQbrain_453_r | 3.84 |
| HS_from_MQbrain_1525_l | 3.83 |
| HS_from_MQbrain_1723_l | 3.83 |
| HS_from_MQbrain_1461_l | 3.83 |
| Hsd_from_Mmd_137_r | 3.80 |
| HSbrain_384_l | 3.79 |
| HS_from_MQbrain_790_r | 3.78 |
| HS_from_MQbrain_329_l | 3.77 |
| HS_from_MQbrain_719_l | 3.75 |
| HSbrain_754_l | 3.73 |
| HSbrain_190_l | 3.72 |
| HSbrain_340_r | 3.71 |
| Hsd_from_Mmd_149_l | 3.70 |
| HS_from_MQbrain_1895_l | 3.68 |
| HS_from_PT_269_l | 3.67 |
| Hsd_from_Mmd_170_r | 3.65 |
| HS_from_MQbrain_637_l | 3.64 |
| HSbrain_20_r | 3.64 |
| HS_from_MQbrain_972_l | 3.63 |
| HSbrain_473_r | 3.62 |
| HSbrain_225_l | 3.62 |
| HS_from_MQbrain_769_r | 3.62 |
| HS_from_MM_50_r | 3.61 |
| Hsd_104_r | 3.60 |
| HSbrain_1386_l | 3.60 |
| HS_from_MQbrain_1197_r | 3.59 |
| Hsd_3_r | 3.58 |
| HS_from_MM_201_l | 3.56 |
| HSbrain_163_r | 3.56 |
| HSbrain_667_l | 3.54 |
| HSbrain_523_r | 3.48 |
| HS_37_r | 3.47 |
| HS_from_MM_86_l | 3.47 |
| Hsd_from_Mmd_187_r | 3.46 |
| HS_from_MQbrain_1791_l | 3.45 |
| HS_from_MQbrain_587_l | 3.45 |
| HS_from_MM_314_l | 3.44 |
| HS_from_MQbrain_1815_r | 3.43 |
| HSbrain_672_l | 3.43 |
| HS_from_MQbrain_1792_r | 3.43 |
| HSbrain_237_r | 3.41 |
| Hsd_from_Mmd_486_r | 3.41 |
| HSbrain_3_r | 3.37 |
| HS_from_MQbrain_279_l | 3.36 |
| Hsd_88_l | 3.35 |
| HSbrain_205_r | 3.35 |
| HS_from_MQbrain_791_l | 3.34 |
| HSbrain_1389_l | 3.34 |
| HS_264_l | 3.32 |
| HS_from_MM_9_r | 3.31 |
| HSbrain_604_r | 3.30 |
| HS_from_MM_268_l | 3.29 |
| HS_from_MQbrain_193_r | 3.28 |
| Hsd_53_l | 3.27 |
| HS_from_MQbrain_314_l | 3.26 |
| Hsd_from_Mmd_328_r | 3.25 |
| Hsd_from_Mmd_426_l | 3.24 |
| HS_from_MQbrain_855_r | 3.24 |
| HS_from_MQbrain_814_r | 3.24 |
| HSbrain_354_r | 3.23 |
| HS_from_MQbrain_732_l | 3.21 |
| HS_from_MM_105_l | 3.20 |
| HSbrain_655_r | 3.20 |
| Hsd_from_Mmd_293_l | 3.20 |
| HS_from_MQbrain_827_r | 3.20 |
| HS_from_MQbrain_910_l | 3.19 |
| HS_from_MQbrain_1256_l | 3.19 |
| HS_from_PT_284_r | 3.19 |
| HS_from_MQbrain_829_l | 3.17 |
| HS_from_MQbrain_367_r | 3.17 |
| HSbrain_544_l | 3.17 |
| HS_302_l | 3.16 |
| HS_from_MM_185_r | 3.15 |
| HS_from_PT_130_r | 3.15 |
| Hsd_from_Mmd_391_l | 3.15 |
| HS_from_MQbrain_1820_r | 3.15 |
| HS_from_MQbrain_1137_r | 3.15 |
| HSbrain_1405_l | 3.14 |
| HS_from_MQbrain_608_r | 3.12 |
| HS_from_MQbrain_1408_l | 3.11 |
| HS_175_l | 3.10 |
| HSbrain_122_r | 3.07 |
| HS_from_PT_104_r | 3.06 |
| Hsd_from_Mmd_272_r | 3.06 |
| HS_from_MQbrain_593_r | 3.02 |
| HS_from_MQbrain_393_l | 2.99 |
| HS_from_MQbrain_854_l | 2.97 |
| HS_from_PT_32_l | 2.96 |
| HSbrain_742_r | 2.95 |
| Hsd_from_Mmd_459_r | 2.93 |
| HS_from_MM_180_l | 2.93 |
| HS_from_MQbrain_1514_r | 2.92 |
| HS_from_PT_257_r | 2.92 |
| HS_301_l | 2.91 |
| Hsd_from_Mmd_509_l | 2.90 |
| HS_from_MQbrain_797_l | 2.90 |
| HS_from_MQbrain_1269_l | 2.89 |
| HS_from_MM_146_l | 2.89 |
| HS_from_PT_154_r | 2.88 |
| HSbrain_247_l | 2.87 |
| HSbrain_423_l | 2.86 |
| Hsd_54_l | 2.85 |
| HS_from_PT_149_r | 2.84 |
| HSbrain_494_l | 2.84 |
| HS_from_MQbrain_906_r | 2.83 |
| HSbrain_755_l | 2.82 |
| HSbrain_541_l | 2.81 |
| HS_from_PT_203_r | 2.80 |
| HS_214_l | 2.79 |
| Hsd_from_Mmd_505_r | 2.79 |
| HS_from_MM_93_l | 2.78 |
| HSbrain_641_r | 2.78 |
| HS_from_MQbrain_140_r | 2.77 |
| HS_from_MQbrain_772_r | 2.77 |
| HS_from_MQbrain_438_r | 2.76 |
| HS_from_MQbrain_389_l | 2.75 |
| HS_205_r | 2.74 |
| HS_from_MQbrain_1428_r | 2.73 |
| HSbrain_1404_l | 2.73 |
| Hsd_85_l | 2.73 |
| HS_from_MM_132_r | 2.73 |
| HS_237_l | 2.70 |
| Hsd_from_Mmd_184_r | 2.70 |
| HSbrain_700_r | 2.69 |
| HSbrain_295_l | 2.68 |
| Hsd_from_Mmd_378_l | 2.68 |
| HS_from_MQbrain_1849_l | 2.67 |
| HS_from_MQbrain_283_l | 2.66 |
| HS_from_MQbrain_55_l | 2.66 |
| Hsd_22_l | 2.62 |
| HS_from_MQbrain_1694_l | 2.61 |
| HS_from_MQbrain_1250_r | 2.61 |
| HS_from_MQbrain_1598_l | 2.61 |
| HS_235_l | 2.61 |
| HSbrain_540_r | 2.58 |
| HS_from_MQbrain_681_l | 2.58 |
| HS_from_MQbrain_1195_r | 2.58 |
| HS_from_MQbrain_1296_l | 2.58 |
| HS_61_r | 2.57 |
| HS_229_l | 2.57 |
| HS_from_MQbrain_1257_l | 2.57 |
| Hsd_33_r | 2.56 |
| HSbrain_671_r | 2.56 |
| Hsd_from_Mmd_525_r | 2.56 |
| Hsd_from_Mmd_51_l | 2.55 |
| HSbrain_545_l | 2.54 |
| HS_from_MQbrain_1778_r | 2.54 |
| Hsd_16_l | 2.53 |
| HS_from_MM_168_l | 2.53 |
| HSbrain_781_l | 2.52 |
| HSbrain_382_l | 2.52 |
| HS_from_MQbrain_1103_r | 2.51 |
| Hsd_from_Mmd_26_r | 2.50 |
| HSbrain_1338_l | 2.50 |
| Hsd_from_Mmd_410_l | 2.50 |
| HSbrain_405_r | 2.49 |
| HSbrain_574_r | 2.49 |
| HSbrain_619_l | 2.49 |
| HS_13_r | 2.45 |
| HSbrain_1417_r | 2.44 |
| HSbrain_543_r | 2.44 |
| HS_from_MQbrain_1114_r | 2.43 |
| HS_127_l | 2.43 |
| HS_from_MQbrain_462_r | 2.41 |
| HS_from_MQbrain_1238_r | 2.40 |
| HS_from_MQbrain_195_l | 2.39 |
| HSbrain_246_r | 2.36 |
| HSbrain_662_r | 2.36 |
| HS_from_PT_271_r | 2.36 |
| HS_from_MQbrain_689_r | 2.35 |
| Hsd_from_Mmd_388_r | 2.35 |
| HSbrain_580_l | 2.35 |
| HSbrain_281_l | 2.34 |
| Hsd_from_Mmd_59_l | 2.33 |
| HS_from_MQbrain_1636_l | 2.33 |
| HS_from_MQbrain_62_l | 2.30 |
| Hsd_from_Mmd_154_r | 2.30 |
| HSbrain_546_r | 2.27 |
| Hsd_30_r | 2.27 |
| HS_from_MM_285_r | 2.25 |
| HS_from_MQbrain_141_l | 2.24 |
| HSbrain_414_r | 2.22 |
| HSbrain_76_l | 2.22 |
| HS_from_MQbrain_176_r | 2.22 |
| HS_223_l | 2.21 |
| Hsd_from_Mmd_159_l | 2.21 |
| HS_from_MM_47_r | 2.21 |
| Hsd_from_Mmd_298_r | 2.19 |
| HS_from_MQbrain_816_l | 2.17 |
| HS_from_MQbrain_1469_r | 2.17 |
| HS_220_l | 2.15 |
| HS_27_l | 2.15 |
| HSbrain_358_l | 2.14 |
| HS_from_MQbrain_144_l | 2.14 |
| HSbrain_1422_l | 2.12 |
| HS_204_r | 2.11 |
| HS_from_PT_128_r | 2.11 |
| HS_from_MM_101_l | 2.10 |
| HS_69_l | 2.10 |
| HS_120_r | 2.10 |
| HS_from_MQbrain_451_l | 2.09 |
| HS_104_l | 2.08 |
| HS_from_MM_116_r | 2.08 |
| HS_from_PT_126_r | 2.08 |
| HS_from_MQbrain_209_l | 2.06 |
| HS_from_MQbrain_1946_l | 2.06 |
| HS_from_MQbrain_1308_r | 2.05 |
| HS_from_MQbrain_334_l | 2.04 |
| HS_from_MQbrain_1444_l | 2.03 |
| HS_from_MQbrain_594_l | 2.01 |
| HS_from_MM_1_r | 2.00 |
| HS_from_MM_181_r | 2.00 |

¹Signal/background cut off > 2

TABLE 14a
Small RNAs recovered from blood and differentially expressed in
adenoma vs normal tissue: hairpin arm with highest expression

| | 1^{st} arm | adenoma vs control |
|---|---|---|
| Systematic name | signal/background¹ | (adj p)² |
| HS_169_l | 138.82 | 0.000489293 |
| HS_141_l | 105.74 | 0.000489293 |
| HS_217_l | 34.38 | 0.000489293 |
| HS_218_r | 2191.38 | 0.000704567 |
| HS_255_l | 144.06 | 0.001129352 |
| HS_232_l | 109.73 | 0.001129352 |
| HS_71_l | 28.7 | 0.003939952 |
| HS_from_MM_309_r | 39.96 | 0.004006907 |
| HS_from_MM_178_r | 3.95 | 0.004006907 |
| HS_from_MM_334_l | 71.92 | 0.004431894 |
| HS_from_MM_319_l | 107.03 | 0.004431894 |
| HS_from_MM_339_r | 2501.25 | 0.004575487 |
| HS_from_MM_63_l | 4.64 | 0.004575487 |
| HS_from_MQbrain_100_l | 62.37 | 0.004575487 |
| HS_from_MQbrain_1043_r | 1537.86 | 0.004799641 |
| HS_from_MQbrain_1056_l | 5.37 | 0.004799641 |
| HS_from_MQbrain_1128_l | 33.94 | 0.004957702 |
| HS_from_MQbrain_1183_l | 112.54 | 0.004957702 |
| HS_from_MQbrain_1100_l | 37.05 | 0.004957702 |
| HS_from_MQbrain_1297_l | 70.63 | 0.006601644 |
| HS_from_MQbrain_1292_r | 11.08 | 0.006601644 |
| HS_from_MQbrain_131_r | 447.53 | 0.00676609 |
| HS_from_MQbrain_1444_l | 2.03 | 0.007781761 |
| HS_from_MQbrain_1456_l | 251.46 | 0.008573112 |
| HS_from_MQbrain_1462_l | 33.63 | 0.008573112 |
| HS_from_MQbrain_1522_r | 7.73 | 0.008946182 |
| HS_from_MQbrain_1485_r | 5.89 | 0.008946182 |
| HS_from_MQbrain_1624_l | 1452.35 | 0.008946182 |
| HS_from_MQbrain_1625_r | 907.04 | 0.008946182 |
| HS_from_MQbrain_1630_l | 1834.56 | 0.008946182 |
| HS_from_MQbrain_1657_r | 10.62 | 0.009734613 |
| HS_from_MQbrain_191_l | 276.03 | 0.01044887 |
| HS_from_MQbrain_1792_r | 3.43 | 0.01044887 |
| HS_from_MQbrain_1823_r | 6.11 | 0.01044887 |
| HS_from_MQbrain_1884_r | 7.89 | 0.01044887 |
| HS_from_MQbrain_1808_r | 172.48 | 0.01044887 |
| HS_from_MQbrain_629_l | 237.88 | 0.010727702 |
| HS_from_MQbrain_330_r | 3085.23 | 0.010727702 |
| HS_from_MQbrain_349_l | 214.68 | 0.010727702 |
| HS_from_MQbrain_55_l | 2.66 | 0.010727702 |
| HS_from_MQbrain_207_l | 1356.12 | 0.010727702 |
| HS_from_MQbrain_20_r | 49.05 | 0.010727702 |
| HS_from_MQbrain_549_l | 5.98 | 0.010727702 |
| HS_from_MQbrain_671_r | 1023.79 | 0.010727702 |
| HS_from_MQbrain_350_l | 188.59 | 0.010727702 |
| HS_from_MQbrain_2380_r | 100.43 | 0.010727702 |
| HS_from_MQbrain_212_r | 7.74 | 0.010727702 |
| HS_from_MQbrain_71_r | 790.88 | 0.010727702 |
| HS_from_MQbrain_619_r | 50.44 | 0.010727702 |
| HS_from_MQbrain_194_l | 16.83 | 0.010727702 |
| HS_from_MQbrain_79_r | 29.36 | 0.011699489 |
| HS_from_MQbrain_839_r | 10.92 | 0.011699489 |
| HS_from_MQbrain_965_l | 243.53 | 0.012506309 |
| HS_from_MQbrain_940_r | 317.39 | 0.012506309 |
| HS_from_MQbrain_917_r | 1391.24 | 0.012506309 |
| HS_from_PT_237_r | 1800.67 | 0.013034759 |
| HS_from_PT_190_r | 545.93 | 0.013034759 |
| HS_from_PT_257_r | 2.92 | 0.013034759 |
| HSbrain_12_l | 6.74 | 0.017198956 |
| HSbrain_171_l | 2560.96 | 0.017198956 |
| HSbrain_1_l | 471.82 | 0.017198956 |
| HSbrain_106_r | 53.44 | 0.017198956 |
| HSbrain_158_l | 43.6 | 0.017198956 |
| HSbrain_1305_l | 237.71 | 0.017198956 |
| HSbrain_148_r | 68.52 | 0.017198956 |
| HSbrain_102_r | 653.85 | 0.017198956 |
| HSbrain_2_l | 84.89 | 0.018600058 |
| HSbrain_253_l | 67.08 | 0.018600058 |
| HSbrain_243_l | 593.95 | 0.018600058 |
| HSbrain_321_r | 587.71 | 0.018600058 |
| HSbrain_224_r | 25.38 | 0.018600058 |
| HSbrain_231_l | 1709.15 | 0.018600058 |
| HSbrain_337_l | 18.24 | 0.019454312 |
| HSbrain_377_r | 3.94 | 0.019454312 |
| HSbrain_426_r | 1715.96 | 0.019454312 |
| HSbrain_420_r | 1374.92 | 0.019454312 |
| HSbrain_359_r | 207.52 | 0.019454312 |
| HSbrain_540_r | 2.58 | 0.021088597 |
| HSbrain_52_r | 24.74 | 0.021088597 |
| HSbrain_503_r | 388.85 | 0.021088597 |
| HSbrain_483_l | 33.11 | 0.021088597 |
| HSbrain_485_r | 2284.67 | 0.021088597 |
| HSbrain_527_r | 1052.64 | 0.021088597 |
| HSbrain_6_r | 2079.02 | 0.022745616 |
| HSbrain_617_l | 106.18 | 0.022745616 |
| HSbrain_597_r | 342.91 | 0.022745616 |
| HSbrain_629_l | 1754.85 | 0.022745616 |
| HSbrain_709_l | 1509.68 | 0.022745616 |
| HSbrain_636_l | 1264.65 | 0.022745616 |
| HSbrain_727_l | 22.41 | 0.023736962 |
| HSbrain_723_l | 277.73 | 0.023736962 |
| Hsd_18_l | 551.25 | 0.023736962 |
| HSbrain_801_r | 81.51 | 0.023736962 |
| HSbrain_8_r | 2366.39 | 0.023736962 |
| HSbrain_75_l | 940.56 | 0.023736962 |
| HSbrain_811_r | 5.93 | 0.023736962 |
| HSbrain_916_l | 55.74 | 0.023736962 |
| HSbrain_722_l | 554.56 | 0.023736962 |
| Hsd_70_l | 685 | 0.024762366 |
| Hsd_from_Mmd_298_r | 2.19 | 0.024762366 |
| Hsd_from_Mmd_527_r | 11.56 | 0.024762366 |
| Hsd_49_r | 2232.71 | 0.024762366 |
| Hsd_from_Mmd_92_r | 34.05 | 0.024762366 |
| Hsd_from_Mmd_533_l | 5.22 | 0.024762366 |
| Hsd_26_l | 6.89 | 0.024762366 |
| HS_from_MQbrain_734_l | 2968.08 | 0.026800941 |
| HS_from_MQbrain_1684_l | 714.03 | 0.026800941 |
| HS_from_MQbrain_509_r | 8.85 | 0.026800941 |
| HS_from_MQbrain_74_r | 1469.53 | 0.028655448 |
| HSbrain_318_l | 2130.43 | 0.028655448 |
| HS_from_PT_139_l | 28.77 | 0.028655448 |
| HS_from_MQbrain_391_r | 53.8 | 0.028655448 |
| HS_from_MQbrain_1832_l | 2323.53 | 0.02997784 |
| HS_from_MQbrain_1169_l | 27.22 | 0.02997784 |
| HSbrain_259_r | 1597.52 | 0.02997784 |
| HS_from_PT_299_l | 1425.1 | 0.02997784 |
| HS_106_l | 55.68 | 0.02997784 |
| HSbrain_546_r | 2.27 | 0.02997784 |
| HSbrain_713_l | 2635.96 | 0.02997784 |
| HS_from_MQbrain_918_l | 604.37 | 0.02997784 |
| HS_from_MQbrain_1443_r | 361.33 | 0.03224042 |
| HS_118_l | 634.82 | 0.03224042 |
| HS_from_MQbrain_1469_r | 2.17 | 0.03224042 |
| HS_from_MQbrain_1392_r | 1984.76 | 0.03224042 |
| HS_from_PT_103_l | 77.68 | 0.03224042 |
| HS_from_MQbrain_1445_l | 67.15 | 0.03224042 |
| HS_from_MQbrain_1863_l | 3259.56 | 0.03224042 |
| HSbrain_316_l | 1216.28 | 0.03374871 |
| HSbrain_41_l | 264.89 | 0.03374871 |
| HS_231_r | 34.84 | 0.03374871 |
| HSbrain_165_l | 137.58 | 0.03374871 |
| HSbrain_458_l | 14.44 | 0.03374871 |
| HS_from_MQbrain_435_l | 14.03 | 0.03374871 |
| Hsd_from_Mmd_81_l | 42.55 | 0.03374871 |
| HS_from_MQbrain_1690_l | 10.98 | 0.03374871 |
| HSbrain_760_l | 60.42 | 0.03374871 |
| HS_from_MQbrain_1340_l | 248.35 | 0.036099692 |
| Hsd_102_l | 51.2 | 0.036099692 |
| HS_from_MQbrain_1513_r | 11.02 | 0.036099692 |
| HSbrain_261_l | 4.72 | 0.038273758 |
| HS_from_MQbrain_498_r | 2610.14 | 0.038273758 |
| HSbrain_543_r | 2.44 | 0.038273758 |
| HS_from_MQbrain_282_l | 762.12 | 0.038273758 |
| HS_from_MQbrain_1315_l | 234.79 | 0.038273758 |
| HSbrain_700_r | 2.69 | 0.038273758 |
| HS_from_MQbrain_470_l | 136.89 | 0.040151024 |
| HS_from_MQbrain_951_l | 216.15 | 0.040151024 |
| HS_from_MQbrain_1080_l | 6.04 | 0.040151024 |
| HS_189_l | 103.99 | 0.040151024 |
| HS_56_r | 793.43 | 0.040151024 |
| HSbrain_92_l | 1202.44 | 0.040151024 |
| HS_from_PT_43_l | 1108.55 | 0.040151024 |
| HSbrain_109_l | 2695.37 | 0.040151024 |
| Hsd_from_Mmd_436_r | 4.74 | 0.040151024 |
| HS_from_MQbrain_1747_r | 1297.11 | 0.040151024 |
| HS_261_r | 142.25 | 0.042549287 |
| Hsd_from_Mmd_261_l | 2305.13 | 0.042549287 |
| HS_from_MQbrain_1227_l | 228.29 | 0.042549287 |
| Hsd_42_r | 755.65 | 0.042549287 |
| HSbrain_1410_l | 22.68 | 0.042549287 |
| HS_from_MQbrain_782_r | 761.75 | 0.042549287 |
| HS_15_l | 316.41 | 0.042549287 |
| HS_from_MQbrain_1388_l | 1165.24 | 0.042549287 |
| HS_from_MQbrain_1844_r | 1717.31 | 0.042549287 |
| HS_266_r | 5.54 | 0.045725655 |
| HSbrain_284_l | 2165.84 | 0.045725655 |
| Hsd_63_r | 1446.53 | 0.045725655 |
| HS_from_PT_70_r | 1095.93 | 0.045725655 |
| Hsd_from_Mmd_34_r | 46.61 | 0.045725655 |
| HSbrain_25_r | 4.72 | 0.048378078 |
| HS_from_MQbrain_1075_l | 2494.05 | 0.048378078 |
| HSbrain_703_l | 1943.04 | 0.048378078 |
| HS_114_l | 715.1 | 0.048378078 |
| HSbrain_1362_l | 962.43 | 0.048378078 |
| HS_from_MQbrain_92_l | 462.65 | 0.048378078 |
| HS_from_MQbrain_1773_l | 479.94 | 0.048378078 |
| HS_from_MM_1_r | 2 | 0.048378078 |
| HS_from_MQbrain_539_r | 1889.5 | 0.048378078 |
| HS_from_MQbrain_472_r | 187.77 | 0.048378078 |
| HS_from_MQbrain_343_l | 224.21 | 0.050130838 |
| HS_from_MQbrain_315_l | 248.59 | 0.050130838 |
| HSbrain_652_l | 1052.64 | 0.050130838 |
| HS_from_MQbrain_572_l | 7.03 | 0.050130838 |
| HS_from_MQbrain_1610_r | 102.62 | 0.050130838 |
| HSbrain_84_l | 13.53 | 0.050130838 |
| HS_from_PT_136_l | 88.09 | 0.050130838 |
| HS_from_MQbrain_1686_l | 2850.33 | 0.050130838 |
| HSbrain_585_l | 70.01 | 0.050130838 |
| HS_from_MQbrain_774_r | 383.95 | 0.050130838 |
| HS_from_MQbrain_901_l | 2410.6 | 0.050130838 |
| HS_215_l | 51.87 | 0.050130838 |
| HS_from_MQbrain_1283_l | 692.85 | 0.050130838 |
| HS_from_MQbrain_1334_r | 10.69 | 0.053159859 |
| HSbrain_442_r | 5.68 | 0.053159859 |
| HS_from_MQbrain_1395_r | 200.62 | 0.053159859 |
| Hsd_37_l | 37.1 | 0.053159859 |
| HS_from_MQbrain_1239_l | 588.81 | 0.053159859 |
| HS_from_MQbrain_873_r | 599.55 | 0.053159859 |
| HS_131_r | 546.58 | 0.053159859 |
| HS_from_MQbrain_1484_l | 1417.92 | 0.053159859 |
| HSbrain_419_l | 1039 | 0.05566524 |
| HS_from_MQbrain_56_l | 125.18 | 0.05566524 |
| HS_62_l | 8.32 | 0.05566524 |
| HS_from_MQbrain_1620_l | 1575.67 | 0.05566524 |
| HS_from_MM_49_l | 28.19 | 0.05566524 |
| HS_from_MQbrain_664_l | 35.08 | 0.05566524 |
| HS_from_MM_115_l | 2053.1 | 0.05566524 |
| HS_59_l | 19.16 | 0.05566524 |
| HSbrain_239_l | 22.71 | 0.05566524 |
| HS_from_MQbrain_1674_l | 6.8 | 0.05566524 |
| HSbrain_85_l | 443.76 | 0.05566524 |
| HSbrain_671_r | 2.56 | 0.05566524 |
| HS_from_MQbrain_974_l | 1938.97 | 0.058253679 |
| HSbrain_123_l | 2450.67 | 0.058253679 |
| HS_from_MQbrain_1374_l | 242.85 | 0.058253679 |
| Hsd_7_r | 7.55 | 0.058253679 |
| HS_from_MQbrain_883_r | 626.09 | 0.058253679 |
| HS_from_MQbrain_1119_l | 2299.12 | 0.058253679 |
| HS_from_PT_121_r | 144.82 | 0.058253679 |
| HS_190_l | 2565.53 | 0.058253679 |
| HS_from_MQbrain_1300_l | 151.72 | 0.058253679 |
| HSbrain_35_r | 185.02 | 0.058253679 |
| HS_from_MQbrain_810_l | 2819.61 | 0.058253679 |
| HS_from_PT_340_l | 74.7 | 0.061046963 |
| HSbrain_214_l | 5.03 | 0.061046963 |
| HS_from_MQbrain_128_r | 205.98 | 0.061046963 |
| HS_from_MQbrain_70_l | 2632.56 | 0.061046963 |
| HS_304_r | 442.22 | 0.061046963 |
| HSbrain_409_r | 1021.91 | 0.061046963 |
| Hsd_from_Mmd_439_l | 141.63 | 0.061046963 |
| HS_36_l | 35.1 | 0.061046963 |
| HS_from_MQbrain_1354_l | 14.56 | 0.061046963 |
| HS_150_l | 1178.4 | 0.061046963 |
| HSbrain_157_l | 60.78 | 0.064770288 |
| HSbrain_755_l | 2.82 | 0.064770288 |
| HSbrain_506_l | 9.24 | 0.064770288 |
| HS_from_MQbrain_750_r | 348.17 | 0.064770288 |
| HS_180_r | 57.76 | 0.064770288 |
| HSbrain_116_l | 61.35 | 0.064770288 |
| Hsd_62_l | 733.75 | 0.067503134 |
| HS_from_MQbrain_239_r | 1469.78 | 0.067503134 |
| HSbrain_330_l | 636.49 | 0.067503134 |
| Hsd_from_Mmd_524_r | 4.21 | 0.067503134 |
| HS_from_MQbrain_745_r | 2408.79 | 0.067503134 |
| HS_203_r | 66.49 | 0.067503134 |
| HS_from_PT_112_l | 2489.56 | 0.067503134 |
| HS_from_PT_339_l | 1393.25 | 0.067503134 |
| HS_from_MQbrain_1427_l | 1542.86 | 0.067503134 |
| HSbrain_212_l | 82.21 | 0.067503134 |
| HSbrain_139_l | 2546.02 | 0.067503134 |
| HSbrain_572_r | 13.46 | 0.067503134 |
| Hsd_from_Mmd_330_l | 1411.51 | 0.069749265 |
| HS_from_MQbrain_1771_r | 1664.95 | 0.069749265 |
| Hsd_50_l | 2796.99 | 0.069749265 |
| HSbrain_657_l | 21.56 | 0.069749265 |
| HS_from_MQbrain_1841_r | 150.14 | 0.069749265 |
| HS_from_MQbrain_723_r | 1023.31 | 0.069749265 |
| HSbrain_245_r | 56.71 | 0.069749265 |
| HSbrain_610_r | 1336.12 | 0.069749265 |
| HS_from_MQbrain_641_r | 14 | 0.069749265 |
| HS_from_MQbrain_997_l | 42.8 | 0.069749265 |
| HS_from_PT_187_l | 13.27 | 0.069749265 |
| HS_from_MQbrain_251_r | 190.83 | 0.069749265 |
| HS_from_MQbrain_1376_l | 937.34 | 0.069749265 |
| HS_from_MQbrain_366_r | 15.16 | 0.069749265 |
| HS_from_MQbrain_1082_l | 744.36 | 0.072349461 |
| HS_from_MQbrain_433_l | 49.52 | 0.072349461 |
| HS_from_MQbrain_1268_l | 6.35 | 0.072349461 |
| HSbrain_1364_l | 1439.39 | 0.072349461 |
| HSbrain_749_r | 545.39 | 0.072349461 |
| HS_from_MM_295_r | 1858.45 | 0.072349461 |
| HSbrain_1363_l | 1163.09 | 0.072349461 |
| HSbrain_747_l | 17.76 | 0.072349461 |
| HS_from_MM_289_r | 153.91 | 0.072349461 |
| HS_from_MQbrain_1561_l | 240.38 | 0.072349461 |
| HS_from_MQbrain_1565_r | 286.84 | 0.072349461 |
| HS_from_MQbrain_1280_l | 891.24 | 0.072349461 |
| HS_from_MQbrain_1219_l | 3215.41 | 0.072349461 |
| Hsd_from_Mmd_333_r | 4.01 | 0.072349461 |
| HS_from_PT_170_l | 2619.23 | 0.072349461 |
| HS_from_MQbrain_1658_r | 23.47 | 0.072349461 |
| HS_from_MQbrain_634_l | 7.38 | 0.076797167 |
| HS_from_MM_219_l | 151.42 | 0.076797167 |
| HS_from_MQbrain_930_r | 8.71 | 0.076797167 |
| HS_from_MQbrain_224_l | 24.13 | 0.076797167 |
| HS_from_MQbrain_687_r | 25.58 | 0.076797167 |
| HSbrain_767_r | 623.64 | 0.076797167 |
| HS_from_MQbrain_1414_r | 7.45 | 0.076797167 |
| Hsd_101_l | 127.99 | 0.079954597 |
| HS_119_l | 2088.91 | 0.079954597 |
| HS_from_MQbrain_1637_r | 961.67 | 0.079954597 |
| HS_from_MQbrain_1777_l | 3386.15 | 0.079954597 |
| HS_223_l | 2.21 | 0.079954597 |
| HSbrain_317_l | 654.52 | 0.079954597 |
| HS_from_MQbrain_1520_r | 49.34 | 0.079954597 |
| HS_from_MQbrain_1706_l | 404.7 | 0.079954597 |
| Hsd_11_l | 114.03 | 0.079954597 |
| HSbrain_455_r | 1213.86 | 0.079954597 |
| HS_from_MQbrain_1700_l | 36.53 | 0.079954597 |
| HS_from_PT_102_l | 2772.91 | 0.079954597 |
| HS_from_MQbrain_576_l | 1930.76 | 0.079954597 |
| HS_from_PT_148_l | 1647.57 | 0.079954597 |
| HS_from_MQbrain_1745_r | 237.34 | 0.079954597 |
| Hsd_from_Mmd_204_l | 52.22 | 0.079954597 |
| HSbrain_389_l | 15.54 | 0.083930001 |
| HS_from_MQbrain_715_l | 2873.52 | 0.083930001 |
| HS_from_MQbrain_1102_l | 2428.54 | 0.083930001 |
| HS_from_MQbrain_769_r | 3.62 | 0.083930001 |
| HS_from_PT_20_r | 1411.59 | 0.083930001 |
| HSbrain_56_l | 2577.8 | 0.083930001 |
| HSbrain_548_l | 2363.14 | 0.083930001 |
| HSbrain_711_r | 9.31 | 0.083930001 |
| HSbrain_286_l | 526.85 | 0.083930001 |
| HS_from_MQbrain_154_l | 198.44 | 0.083930001 |
| HS_57_l | 1000.27 | 0.083930001 |
| HS_from_MM_202_l | 128.65 | 0.083930001 |
| HS_from_MQbrain_1325_r | 73.72 | 0.083930001 |
| HS_from_MM_195_r | 648.55 | 0.083930001 |
| Hsd_from_Mmd_501_r | 125.01 | 0.083930001 |
| HS_from_MQbrain_686_r | 2145.46 | 0.088143181 |
| HS_102_l | 2248.72 | 0.088143181 |
| HS_from_PT_254_r | 11.89 | 0.088143181 |
| HS_from_MQbrain_1064_r | 37.45 | 0.088143181 |
| HSbrain_590_r | 254.85 | 0.088143181 |
| HSbrain_124_l | 194.72 | 0.088143181 |
| HS_from_MQbrain_948_r | 210.86 | 0.088143181 |
| HS_11_l | 6.83 | 0.088143181 |
| HS_from_MM_347_l | 2499.97 | 0.088143181 |
| HSbrain_170_r | 4.6 | 0.088143181 |
| HS_80_l | 19.03 | 0.088143181 |
| HS_from_MM_165_l | 7.64 | 0.088143181 |
| HS_from_MQbrain_806_l | 201.32 | 0.088143181 |
| HS_from_MQbrain_291_l | 819.28 | 0.088143181 |
| Hsd_from_Mmd_378_l | 2.68 | 0.09152926 |
| Hsd_from_Mmd_120_r | 1906.46 | 0.09152926 |
| HS_from_MQbrain_550_l | 2602.74 | 0.09152926 |
| HSbrain_237_r | 3.41 | 0.09152926 |
| HS_from_PT_95_r | 744.48 | 0.09152926 |
| HS_from_MQbrain_1885_r | 344.16 | 0.09152926 |
| HSbrain_1360_l | 1251.79 | 0.09152926 |
| HS_from_MQbrain_1758_r | 2139.74 | 0.09152926 |
| HS_from_MQbrain_1592_l | 38.47 | 0.09152926 |
| HSbrain_642_r | 31.13 | 0.09152926 |
| HS_4_r | 293.08 | 0.09152926 |
| HSbrain_637_l | 190.08 | 0.09152926 |
| Hsd_from_Mmd_161_l | 62.81 | 0.09152926 |
| HSbrain_708_l | 2404.97 | 0.095394629 |
| HSbrain_691_l | 1101.52 | 0.095394629 |
| HSbrain_717_l | 2399.92 | 0.095394629 |
| HS_288_l | 1661.49 | 0.095394629 |
| HSbrain_113_r | 77.28 | 0.095394629 |
| HSbrain_557_r | 60.82 | 0.095394629 |
| Hsd_79_l | 115.62 | 0.095394629 |
| HS_from_MQbrain_255_l | 53.09 | 0.095394629 |
| Hsd_from_Mmd_519_r | 95.12 | 0.095394629 |
| HS_129_l | 1787.42 | 0.095394629 |
| HSbrain_453_r | 26.71 | 0.095394629 |
| HS_from_MQbrain_1228_r | 2260.21 | 0.095394629 |
| HS_from_PT_74_r | 68.99 | 0.095394629 |
| HSbrain_1425_l | 1793.99 | 0.098921026 |
| HS_from_MM_103_l | 2974.49 | 0.098921026 |
| HS_from_MQbrain_574_l | 439.75 | 0.098921026 |
| HSbrain_305_r | 2302.79 | 0.098921026 |
| HS_from_MQbrain_868_l | 251.95 | 0.098921026 |
| HS_from_MQbrain_2670_r | 19.49 | 0.098921026 |
| Hsd_from_Mmd_480_r | 1038.29 | 0.098921026 |
| Hsd_from_Mmd_176_l | 64.49 | 0.098921026 |
| HSbrain_769_r | 3201.06 | 0.098921026 |
| HS_157_l | 2437.27 | 0.098921026 |
| HSbrain_86_r | 5.53 | 0.098921026 |
| HSbrain_240_l | 2736.22 | 0.098921026 |
| HS_from_MQbrain_1410_l | 8.14 | 0.098921026 |
| HS_from_MQbrain_1213_l | 455.52 | 0.098921026 |
| HS_from_MQbrain_1382_l | 661.03 | 0.098921026 |
| HSbrain_415_l | 403.23 | 0.098921026 |
| HS_from_MQbrain_1355_r | 2362.21 | 0.103393572 |
| HSbrain_677_l | 2284.34 | 0.103393572 |
| HS_from_MQbrain_609_l | 1532.45 | 0.103393572 |
| Hsd_111_r | 92.66 | 0.103393572 |
| HS_from_MQbrain_1459_l | 154.95 | 0.103393572 |
| HSbrain_482_l | 36.52 | 0.103393572 |
| HS_from_MQbrain_1784_r | 278.43 | 0.103393572 |
| HS_68_l | 66.74 | 0.103393572 |
| HSbrain_504_r | 5.59 | 0.103393572 |
| HS_from_MQbrain_906_r | 2.83 | 0.103393572 |
| HS_from_MQbrain_943_l | 1120.64 | 0.103393572 |
| HS_from_PT_22_l | 15.15 | 0.103393572 |
| HSbrain_94_l | 4.24 | 0.103393572 |
| HSbrain_596_r | 151.36 | 0.103393572 |
| HS_from_MQbrain_1744_r | 199.26 | 0.103393572 |
| HSbrain_384_l | 3.79 | 0.108118292 |
| HS_101_l | 2303.1 | 0.108118292 |
| HS_from_MQbrain_437_l | 15.23 | 0.108118292 |
| HSbrain_643_l | 1935.82 | 0.108118292 |
| Hsd_from_Mmd_478_l | 40.92 | 0.108118292 |
| HS_from_MQbrain_546_l | 1884.2 | 0.108118292 |
| HS_from_MQbrain_374_r | 481.44 | 0.108118292 |
| Hsd_from_Mmd_249_r | 5.05 | 0.108118292 |
| HS_from_MQbrain_1627_l | 1294.42 | 0.108118292 |
| HS_164_l | 221 | 0.108118292 |
| HSbrain_536_r | 1895.91 | 0.108118292 |
| HS_from_MQbrain_114_r | 2018.35 | 0.108118292 |
| HS_from_MQbrain_80_l | 35.53 | 0.108118292 |
| HSbrain_280_l | 571.68 | 0.108118292 |
| HS_from_PT_7_r | 19.85 | 0.108118292 |
| HS_from_MQbrain_1366_l | 1085.63 | 0.108118292 |
| HS_from_PT_220_l | 1192.62 | 0.108118292 |
| Hsd_from_Mmd_291_r | 2468.16 | 0.108118292 |
| Hsd_from_Mmd_391_l | 3.15 | 0.113099484 |
| HSbrain_328_l | 3271.08 | 0.113099484 |
| HSbrain_764_r | 42.39 | 0.113099484 |
| HS_from_MQbrain_354_r | 14.17 | 0.113099484 |
| HS_from_MQbrain_921_l | 1527.75 | 0.113099484 |
| HS_from_MQbrain_604_l | 2116.19 | 0.113099484 |
| HSbrain_520_l | 536.06 | 0.113099484 |
| HS_from_MQbrain_1502_l | 10.78 | 0.113099484 |
| HSbrain_412_l | 28.22 | 0.113099484 |
| HSbrain_227_r | 1702.05 | 0.113099484 |
| HS_from_MQbrain_1478_l | 63.78 | 0.113099484 |
| HSbrain_267_l | 604.78 | 0.113099484 |
| HS_from_MQbrain_1750_r | 2658.65 | 0.11858389 |
| Hsd_from_Mmd_518_r | 1496.12 | 0.11858389 |
| HSbrain_383_l | 283.64 | 0.11858389 |
| HS_from_MQbrain_1889_l | 2376.83 | 0.11858389 |
| HS_from_MQbrain_1834_r | 2219.56 | 0.11858389 |
| HSbrain_145_l | 1928.15 | 0.11858389 |
| Hsd_from_Mmd_93_l | 182.17 | 0.11858389 |
| HS_from_MQbrain_999_l | 2142.82 | 0.11858389 |
| HS_from_MQbrain_592_l | 331.9 | 0.11858389 |
| HS_289_l | 1312.22 | 0.11858389 |
| HS_from_MQbrain_1590_l | 189.53 | 0.11858389 |
| HSbrain_664_r | 3210.45 | 0.11858389 |
| HS_from_MQbrain_502_r | 36.99 | 0.11858389 |
| Hsd_from_Mmd_372_l | 10.75 | 0.11858389 |
| HS_from_MQbrain_1487_r | 8.21 | 0.11858389 |
| Hsd_from_Mmd_328_r | 3.25 | 0.12422104 |
| Hsd_from_Mmd_237_l | 2452.36 | 0.12422104 |
| HSbrain_50_l | 1128.11 | 0.12422104 |
| HS_98_r | 2382.13 | 0.12422104 |
| HSbrain_570_l | 1042.7 | 0.12422104 |
| HS_287_l | 1563.84 | 0.12422104 |
| HS_from_MM_79_l | 1394.17 | 0.12422104 |
| Hsd_17_l | 546.42 | 0.12422104 |
| HS_from_MQbrain_1384_r | 5.51 | 0.12422104 |
| HS_from_MQbrain_1109_l | 18.59 | 0.12422104 |
| HS_from_MQbrain_490_r | 269.59 | 0.12422104 |
| HS_from_MM_87_l | 20.41 | 0.12422104 |
| HS_from_MQbrain_1184_r | 434.9 | 0.12422104 |
| HS_25_r | 2127.91 | 0.12422104 |
| HSbrain_131_l | 33.57 | 0.12422104 |
| HS_from_MQbrain_789_l | 1550.08 | 0.12422104 |
| HS_from_MQbrain_1304_r | 84.49 | 0.12422104 |
| Hsd_from_Mmd_183_r | 39.66 | 0.12422104 |
| HSbrain_204_r | 1075.46 | 0.129399391 |
| HSbrain_695_l | 10.34 | 0.129399391 |
| HSbrain_72_l | 1994.62 | 0.129399391 |
| HS_from_MQbrain_1839_l | 2242.94 | 0.129399391 |
| HS_from_MQbrain_1722_l | 121.03 | 0.129399391 |
| HS_from_MQbrain_36_r | 1728.75 | 0.129399391 |
| HS_109_l | 502.5 | 0.129399391 |
| HS_from_MQbrain_137_r | 15.61 | 0.129399391 |
| HS_85_l | 37.14 | 0.129399391 |
| HS_from_MQbrain_1794_r | 837.36 | 0.129399391 |
| HS_from_MQbrain_1776_l | 568.88 | 0.129399391 |
| HSbrain_152_l | 2922.92 | 0.129399391 |
| HS_from_MQbrain_1574_l | 2453.2 | 0.129399391 |
| HS_from_MM_304_r | 1888.94 | 0.129399391 |
| HSbrain_448_l | 51.31 | 0.129399391 |
| HS_from_MQbrain_1097_r | 22.37 | 0.134970683 |
| HS_from_MQbrain_1692_l | 71.86 | 0.134970683 |
| HS_from_MQbrain_1318_l | 327.82 | 0.134970683 |
| HS_from_MQbrain_540_l | 1590.52 | 0.134970683 |
| HS_from_MQbrain_99_r | 1728.73 | 0.134970683 |
| HS_from_PT_250_r | 597.57 | 0.134970683 |
| HS_from_MQbrain_1523_r | 2731.64 | 0.134970683 |
| HS_from_MQbrain_234_r | 2211.14 | 0.134970683 |
| Hsd_from_Mmd_101_r | 1150.48 | 0.134970683 |
| Hsd_74_r | 170.76 | 0.134970683 |
| HS_from_MM_185_r | 3.15 | 0.134970683 |
| HS_219_l | 2255.05 | 0.140323812 |
| HSbrain_361_l | 2360.92 | 0.140323812 |
| HS_from_MQbrain_22_r | 1898.63 | 0.140323812 |
| HS_175_l | 3.1 | 0.140323812 |
| HSbrain_108_l | 923.37 | 0.140323812 |
| HSbrain_716_l | 1552.16 | 0.140323812 |
| HS_from_MQbrain_1855_l | 1224.15 | 0.140323812 |
| HS_from_MQbrain_467_l | 182.78 | 0.140323812 |
| HS_41_r | 18.13 | 0.140323812 |
| HS_121_r | 1207.85 | 0.140323812 |
| HS_168_r | 2155.79 | 0.140323812 |
| HS_from_PT_178_l | 2486.13 | 0.140323812 |
| HSbrain_144_r | 395.32 | 0.140323812 |
| Hsd_from_Mmd_123_l | 362.29 | 0.140323812 |
| HS_53_r | 582.76 | 0.140323812 |
| HSbrain_737_r | 30.44 | 0.140323812 |
| HS_161_r | 9.51 | 0.140323812 |
| HS_from_MQbrain_1675_r | 791.8 | 0.146822481 |
| HS_from_MQbrain_1493_r | 472.59 | 0.146822481 |
| HSbrain_624_l | 1034.54 | 0.146822481 |
| HS_from_MQbrain_97_r | 346.64 | 0.146822481 |
| HSbrain_705_r | 64.83 | 0.146822481 |
| HS_from_PT_219_l | 1425.59 | 0.146822481 |
| HS_from_MQbrain_260_r | 554.62 | 0.146822481 |
| HS_from_MQbrain_1222_r | 2381.15 | 0.146822481 |
| HS_from_PT_284_r | 3.19 | 0.146822481 |
| HSbrain_663_r | 10.59 | 0.146822481 |
| HS_from_MQbrain_67_l | 1010.93 | 0.146822481 |
| HSbrain_130_l | 610.32 | 0.146822481 |
| HS_from_MQbrain_1695_r | 1101.1 | 0.146822481 |
| HS_from_MQbrain_1036_r | 1242.65 | 0.146822481 |
| HS_from_MQbrain_1807_l | 413.66 | 0.146822481 |
| Hsd_77_r | 247.78 | 0.146822481 |
| HS_from_MQbrain_1030_l | 104.18 | 0.146822481 |
| HS_from_MQbrain_1250_r | 2.61 | 0.146822481 |
| HSbrain_306_r | 2986.89 | 0.152858542 |
| HS_from_PT_238_l | 2054.08 | 0.152858542 |
| HS_from_PT_92_l | 2739.09 | 0.152858542 |
| Hsd_from_Mmd_280_l | 4.61 | 0.152858542 |
| HS_from_MQbrain_153_l | 6.19 | 0.152858542 |
| HSbrain_604_r | 3.3 | 0.152858542 |
| HS_from_MQbrain_707_l | 1212.83 | 0.152858542 |
| HSbrain_266_l | 2143.43 | 0.152858542 |
| HSbrain_641_r | 2.78 | 0.152858542 |
| Hsd_38_r | 1037.46 | 0.152858542 |
| HS_from_MQbrain_969_r | 424.82 | 0.152858542 |
| HS_from_MQbrain_1072_r | 7.84 | 0.152858542 |
| HS_from_MQbrain_355_r | 28.65 | 0.152858542 |
| HSbrain_752_l | 556.1 | 0.152858542 |
| HS_from_MQbrain_693_r | 828.02 | 0.152858542 |
| HS_from_MQbrain_631_l | 1729.43 | 0.158799149 |
| HSbrain_783_l | 2871.64 | 0.158799149 |
| HS_from_MM_118_l | 5.89 | 0.158799149 |
| HS_from_MQbrain_1204_r | 2072.07 | 0.158799149 |
| HSbrain_673_l | 2432.23 | 0.158799149 |
| HSbrain_347_l | 1854.13 | 0.158799149 |
| HS_from_PT_158_l | 2670.87 | 0.158799149 |
| HS_from_MQbrain_625_l | 19.94 | 0.158799149 |
| HS_from_MQbrain_1018_l | 2246.41 | 0.158799149 |
| HSbrain_121_l | 293.17 | 0.158799149 |
| HS_from_MQbrain_382_r | 26.42 | 0.158799149 |
| HS_from_MQbrain_6_l | 840.88 | 0.158799149 |
| HS_18_l | 1388.2 | 0.158799149 |
| HS_from_MQbrain_1501_r | 1204.24 | 0.158799149 |
| HS_from_PT_255_l | 634.81 | 0.158799149 |
| HS_from_MQbrain_1831_r | 297.75 | 0.158799149 |
| HSbrain_248_l | 258.9 | 0.158799149 |
| HS_from_MQbrain_414_l | 212.24 | 0.158799149 |
| Hsd_67_r | 27.78 | 0.158799149 |
| HS_from_MM_35_l | 8.96 | 0.158799149 |
| HSbrain_95_l | 464.25 | 0.163891418 |
| HS_from_MQbrain_996_l | 2730.34 | 0.163891418 |
| Hsd_from_Mmd_52_r | 145.37 | 0.163891418 |
| HSbrain_402_l | 2283.48 | 0.163891418 |
| Hsd_from_Mmd_94_l | 10 | 0.163891418 |
| HS_from_MQbrain_824_r | 1660.01 | 0.163891418 |
| HSbrain_304_l | 60.38 | 0.163891418 |
| HS_from_MQbrain_529_r | 1669.81 | 0.163891418 |
| HS_from_MQbrain_1028_l | 164.03 | 0.163891418 |
| HS_from_MQbrain_650_r | 195.56 | 0.163891418 |
| HS_from_MQbrain_1721_l | 944.26 | 0.163891418 |
| HS_from_MQbrain_449_l | 1928.06 | 0.163891418 |
| HS_from_PT_196_r | 10.79 | 0.163891418 |
| HSbrain_680_l | 2680.84 | 0.163891418 |
| Hsd_108_r | 171.48 | 0.163891418 |
| HS_from_PT_234_l | 109.5 | 0.163891418 |
| HSbrain_784_r | 227.25 | 0.163891418 |
| HS_from_MQbrain_1724_l | 37.26 | 0.163891418 |
| HS_from_MQbrain_1426_l | 3397.58 | 0.163891418 |
| HS_from_MQbrain_566_r | 2574.83 | 0.163891418 |
| HS_from_MQbrain_1223_l | 1386.53 | 0.163891418 |
| HS_from_MQbrain_936_r | 152.19 | 0.163891418 |
| HS_from_MQbrain_596_r | 5.84 | 0.163891418 |
| HS_197_r | 2713.25 | 0.170271053 |
| HS_212_r | 82.75 | 0.170271053 |
| HSbrain_467_l | 2150.69 | 0.170271053 |
| HS_from_MQbrain_281_l | 1617.63 | 0.170271053 |
| HS_from_MQbrain_1846_r | 1712.53 | 0.170271053 |
| Hsd_from_Mmd_410_l | 2.5 | 0.170271053 |
| HS_from_MQbrain_468_l | 45.02 | 0.170271053 |
| HS_from_MQbrain_1377_l | 4.44 | 0.170271053 |
| HS_128_r | 595.8 | 0.170271053 |
| HSbrain_216_r | 2537.24 | 0.170271053 |
| HSbrain_143_l | 2228.78 | 0.170271053 |
| HSbrain_53_l | 2212.76 | 0.170271053 |
| HS_from_MQbrain_1199_l | 2060.7 | 0.170271053 |
| HSbrain_701_l | 1684.34 | 0.170271053 |
| HS_from_MQbrain_841_l | 101.61 | 0.170271053 |
| Hsd_from_Mmd_235_r | 92.57 | 0.170271053 |
| HS_192_r | 43.57 | 0.170271053 |
| Hsd_from_Mmd_367_l | 13.74 | 0.170271053 |
| Hsd_from_Mmd_35_l | 7.49 | 0.170271053 |
| HS_from_MQbrain_984_l | 6.6 | 0.176183859 |
| HS_from_PT_27_r | 1359.52 | 0.176183859 |
| HS_from_MQbrain_628_l | 20.61 | 0.176183859 |
| HS_from_MQbrain_314_l | 3.26 | 0.176183859 |
| HSbrain_771_l | 1417.26 | 0.176183859 |
| HS_204_r | 2.11 | 0.176183859 |
| HS_220_l | 2.15 | 0.176183859 |
| HSbrain_32_l | 1943.03 | 0.176183859 |
| HS_from_MQbrain_855_r | 3.24 | 0.176183859 |
| Hsd_65_r | 258.71 | 0.176183859 |
| HSbrain_626_l | 1507.69 | 0.176183859 |
| Hsd_from_Mmd_344_r | 149.5 | 0.176183859 |
| HS_186_r | 242.25 | 0.176183859 |
| HS_from_MQbrain_135_l | 42.75 | 0.176183859 |
| HSbrain_707_r | 9.72 | 0.176183859 |
| HS_from_MM_123_l | 7.67 | 0.176183859 |
| HS_from_MQbrain_361_l | 3.9 | 0.182758186 |
| HSbrain_388_r | 1236.45 | 0.182758186 |
| HS_132_l | 3.95 | 0.182758186 |
| HS_from_MM_6_l | 372.25 | 0.182758186 |
| HS_from_MQbrain_881_l | 29.79 | 0.182758186 |
| HS_from_MM_124_r | 5.3 | 0.182758186 |
| HS_from_MQbrain_1380_r | 2936.43 | 0.182758186 |
| HS_from_MQbrain_1509_r | 2861.75 | 0.182758186 |
| HS_from_MQbrain_1349_l | 2206.2 | 0.182758186 |
| HS_from_MQbrain_162_r | 1795.5 | 0.182758186 |
| HS_from_MQbrain_850_l | 1651.86 | 0.182758186 |
| HS_from_MQbrain_676_r | 14.81 | 0.182758186 |
| HS_from_MM_149_r | 4.41 | 0.182758186 |
| HS_from_MM_160_l | 4.14 | 0.182758186 |
| HSbrain_706_l | 4.08 | 0.182758186 |
| Hsd_from_Mmd_187_r | 3.46 | 0.182758186 |
| HS_from_MM_86_l | 3.47 | 0.188867139 |
| HSbrain_198_l | 7.92 | 0.188867139 |
| HSbrain_571_l | 5.09 | 0.188867139 |
| HSbrain_627_r | 27.61 | 0.188867139 |
| HS_77_l | 1287.06 | 0.188867139 |
| HSbrain_210_l | 2938.2 | 0.188867139 |
| HSbrain_553_l | 12.16 | 0.188867139 |
| HS_from_PT_271_r | 2.36 | 0.188867139 |
| HS_from_MQbrain_487_r | 3196.14 | 0.188867139 |
| HS_from_MQbrain_1073_r | 2353.25 | 0.188867139 |
| HS_from_MQbrain_1596_r | 2252.81 | 0.188867139 |
| HS_from_PT_328_l | 317.19 | 0.188867139 |
| HS_from_MM_182_r | 88.5 | 0.188867139 |
| HS_from_MQbrain_1734_l | 26.58 | 0.188867139 |
| HS_from_MQbrain_1208_l | 19.05 | 0.188867139 |
| HSbrain_370_r | 9.51 | 0.188867139 |
| HS_from_MQbrain_1760_r | 9.4 | 0.188867139 |
| HS_from_MQbrain_1234_l | 4.28 | 0.188867139 |
| HS_from_MQbrain_1424_r | 11.94 | 0.19639408 |
| Hsd_from_Mmd_326_l | 9.49 | 0.19639408 |
| HS_from_PT_130_r | 3.15 | 0.19639408 |
| HS_124_l | 223.23 | 0.19639408 |
| Hsd_19_l | 198.35 | 0.19639408 |
| HS_from_MQbrain_648_l | 898.68 | 0.19639408 |
| HS_198_l | 2797.81 | 0.19639408 |
| HS_from_PT_204_r | 2648.95 | 0.19639408 |
| HSbrain_810_l | 2281.81 | 0.19639408 |
| HSbrain_70_r | 1587.77 | 0.19639408 |
| HS_from_MQbrain_406_r | 1158.97 | 0.19639408 |
| HSbrain_616_r | 260.24 | 0.19639408 |
| HS_from_MQbrain_383_l | 120.82 | 0.19639408 |
| HS_from_MQbrain_1883_r | 6.04 | 0.19639408 |
| HS_from_MQbrain_389_l | 2.75 | 0.19639408 |

¹Cut off Signal/background>2
²Cut off adj p < 0.2

TABLE 14b
Small RNAs recovered from blood and differentially expressed in
adenoma vs normal tissue: hairpin arm with lowest expression

| | 2^{nd} arm | adenoma vs control |
|---|---|---|
| Systematic name | signal/background¹ | (adj p)² |
| HS_21_r | 99.11 | 0.000489293 |
| HS_215_r | 6.03 | 0.000489293 |
| HS_291_r | 4.17 | 0.001647426 |
| HS_9_r | 15.33 | 0.003939952 |
| HS_from_MQbrain_1103_l | 2.46 | 0.004957702 |
| HS_from_MQbrain_1393_r | 240.51 | 0.00676609 |
| HS_from_MQbrain_2700_r | 3.75 | 0.010727702 |
| HS_from_MQbrain_825_r | 5.01 | 0.011699489 |
| HS_from_PT_26_r | 5.11 | 0.013034759 |
| HSbrain_1437_r | 4.06 | 0.017198956 |
| HSbrain_268_r | 27.49 | 0.018600058 |
| HSbrain_362_r | 41.61 | 0.019454312 |
| HSbrain_472_l | 2.61 | 0.019454312 |
| HSbrain_458_r | 2.23 | 0.019454312 |
| HSbrain_713_r | 3.19 | 0.022745616 |
| HSbrain_703_r | 2.01 | 0.022745616 |
| HSbrain_794_r | 128.92 | 0.023736962 |
| HSbrain_778_r | 5.4 | 0.023736962 |
| Hsd_107_l | 3.8 | 0.023736962 |
| Hsd_from_Mmd_219_r | 5.96 | 0.024762366 |
| Hsd_from_Mmd_519_l | 3.96 | 0.024762366 |
| Hsd_from_Mmd_155_l | 2.23 | 0.024762366 |
| HS_from_MQbrain_800_r | 17.09 | 0.03374871 |
| HS_from_PT_295_r | 2.59 | 0.036099692 |
| HS_from_MQbrain_1561_r | 7.06 | 0.040151024 |
| HSbrain_752_r | 43.13 | 0.042549287 |
| HS_from_MM_289_l | 5.4 | 0.042549287 |
| HS_from_MQbrain_1143_r | 47.51 | 0.045725655 |
| HSbrain_48_r | 18.16 | 0.048378078 |
| HS_from_MQbrain_1752_l | 3.64 | 0.048378078 |
| HS_from_MQbrain_1006_l | 3.57 | 0.048378078 |
| HS_from_MQbrain_1771_l | 115.56 | 0.050130838 |
| HS_from_MQbrain_691_l | 50.85 | 0.050130838 |
| HS_from_MQbrain_1642_l | 2.65 | 0.050130838 |
| Hsd_65_l | 2.04 | 0.050130838 |
| HS_from_MQbrain_1794_l | 7.63 | 0.053159859 |
| HSbrain_374_r | 4.56 | 0.053159859 |
| Hsd_76_r | 4.51 | 0.053159859 |
| Hsd_18_r | 2.34 | 0.053159859 |
| HSbrain_916_r | 3.45 | 0.05566524 |
| HSbrain_300_l | 16.46 | 0.058253679 |
| HS_156_l | 7.17 | 0.058253679 |
| HSbrain_304_r | 6.39 | 0.058253679 |
| HS_from_MQbrain_1404_r | 6.61 | 0.061046963 |
| HSbrain_776_l | 156.06 | 0.064770288 |
| HS_from_MQbrain_1696_l | 43.62 | 0.064770288 |
| HS_from_MQbrain_1450_r | 35.38 | 0.064770288 |
| HS_from_PT_41_r | 32.51 | 0.064770288 |
| Hsd_42_l | 3.47 | 0.067503134 |
| HS_from_PT_235_l | 23.13 | 0.069749265 |
| HS_from_MQbrain_37_r | 13.27 | 0.069749265 |
| HS_98_l | 38.97 | 0.072349461 |
| HS_81_r | 30.21 | 0.072349461 |
| HSbrain_301_l | 16.09 | 0.072349461 |
| HSbrain_635_l | 6.83 | 0.072349461 |
| HS_from_MQbrain_253_r | 5.37 | 0.072349461 |
| HSbrain_788_l | 19.27 | 0.076797167 |
| HS_166_r | 4.11 | 0.076797167 |
| HS_from_MQbrain_1665_r | 6.67 | 0.079954597 |
| HSbrain_172_l | 3.38 | 0.079954597 |
| HSbrain_661_l | 2.82 | 0.079954597 |
| HS_from_MQbrain_48_r | 16.99 | 0.083930001 |
| HS_from_MQbrain_113_r | 7.1 | 0.083930001 |
| HS_from_MM_229_r | 74.93 | 0.088143181 |
| HSbrain_122_l | 3.01 | 0.088143181 |
| HS_from_MQbrain_1031_l | 34.93 | 0.09152926 |
| HS_from_MQbrain_1678_r | 18.12 | 0.09152926 |
| HS_from_MQbrain_1602_l | 17.41 | 0.09152926 |
| HS_from_MQbrain_487_l | 9.22 | 0.09152926 |
| HSbrain_19_l | 6.76 | 0.09152926 |
| HS_from_MQbrain_1726_l | 4.39 | 0.09152926 |
| HS_from_MQbrain_1037_r | 2.65 | 0.09152926 |
| Hsd_from_Mmd_371_r | 2.09 | 0.09152926 |
| HS_173_l | 40.18 | 0.095394629 |
| HS_from_MQbrain_932_l | 13.46 | 0.095394629 |
| HS_from_MQbrain_755_l | 8.96 | 0.095394629 |
| HS_from_MQbrain_1201_r | 5.23 | 0.095394629 |
| HS_38_r | 10.14 | 0.098921026 |
| HSbrain_680_r | 8.65 | 0.098921026 |
| HS_from_MQbrain_1139_r | 2.47 | 0.098921026 |
| HS_from_MQbrain_751_l | 41.92 | 0.103393572 |
| Hsd_63_l | 41.23 | 0.103393572 |
| HS_from_MQbrain_1633_r | 9.89 | 0.103393572 |
| HSbrain_673_r | 9.27 | 0.103393572 |
| HSbrain_684_r | 8.86 | 0.103393572 |
| HSbrain_319_l | 6.7 | 0.103393572 |
| HSbrain_299_r | 32.14 | 0.108118292 |
| HS_from_PT_95_l | 8.24 | 0.108118292 |
| HS_from_MQbrain_824_l | 17.99 | 0.113099484 |
| HSbrain_47_r | 9.58 | 0.113099484 |
| HSbrain_216_l | 5.87 | 0.113099484 |
| HSbrain_390_l | 3.48 | 0.113099484 |
| HS_from_MQbrain_241_l | 3.41 | 0.113099484 |
| Hsd_60_r | 11.8 | 0.11858389 |
| HS_from_MQbrain_560_r | 9.17 | 0.11858389 |
| HS_from_MQbrain_301_l | 4.97 | 0.11858389 |
| HS_from_PT_237_l | 42.41 | 0.12422104 |
| HS_from_MQbrain_485_l | 23.03 | 0.12422104 |
| HS_135_l | 15.42 | 0.12422104 |
| HS_from_MQbrain_406_l | 7.81 | 0.12422104 |
| HS_from_PT_283_r | 7.34 | 0.12422104 |
| HS_from_MQbrain_1712_r | 154.76 | 0.129399391 |
| HSbrain_267_r | 33.77 | 0.129399391 |
| HS_from_MQbrain_466_l | 11.16 | 0.129399391 |
| HSbrain_347_r | 8.32 | 0.129399391 |
| HS_from_MQbrain_129_r | 6.71 | 0.129399391 |
| HS_from_PT_207_l | 4.65 | 0.129399391 |
| HS_from_MQbrain_967_l | 2.73 | 0.129399391 |
| HS_from_MQbrain_382_l | 2.37 | 0.129399391 |
| HS_from_MQbrain_684_r | 403 | 0.134970683 |
| Hsd_from_Mmd_291_l | 96.8 | 0.134970683 |
| HSbrain_687_r | 7.98 | 0.134970683 |
| HS_from_MQbrain_1640_l | 5.27 | 0.134970683 |
| HS_from_MQbrain_968_l | 4.35 | 0.134970683 |
| HS_51_r | 2.98 | 0.134970683 |
| HSbrain_126_r | 43.17 | 0.140323812 |
| HS_from_MQbrain_56_r | 4.85 | 0.140323812 |
| HS_157_r | 3.41 | 0.140323812 |
| HS_from_MQbrain_1759_l | 748.44 | 0.146822481 |
| HSbrain_107_r | 10.31 | 0.146822481 |
| HS_from_MQbrain_1462_r | 5.15 | 0.146822481 |
| HSbrain_139_r | 3.45 | 0.146822481 |
| HS_from_MQbrain_6_r | 318.83 | 0.152858542 |
| HSbrain_32_r | 19.15 | 0.152858542 |
| HS_from_MQbrain_340_l | 7.36 | 0.152858542 |
| HSbrain_1325_r | 5.49 | 0.152858542 |
| HS_from_MQbrain_154_r | 3.24 | 0.152858542 |
| HS_176_l | 2.96 | 0.152858542 |
| HSbrain_653_l | 76.21 | 0.158799149 |
| HS_from_MQbrain_281_r | 11.13 | 0.158799149 |
| HSbrain_669_r | 8.92 | 0.158799149 |
| HS_128_l | 8.45 | 0.158799149 |
| HS_from_MQbrain_1090_r | 156.7 | 0.163891418 |
| HS_from_PT_280_l | 18.01 | 0.163891418 |
| HS_from_MQbrain_1520_l | 9.18 | 0.163891418 |
| HS_from_MQbrain_1535_r | 8.09 | 0.163891418 |
| Hsd_84_r | 4.61 | 0.163891418 |
| HS_from_MQbrain_629_r | 4.6 | 0.163891418 |
| HSbrain_578_l | 3.13 | 0.163891418 |
| HS_from_PT_155_l | 2.23 | 0.163891418 |
| HSbrain_745_l | 1921.15 | 0.170271053 |
| HSbrain_364_r | 156.81 | 0.170271053 |
| HS_from_MM_304_l | 93.08 | 0.170271053 |
| HSbrain_724_l | 18.81 | 0.170271053 |
| HSbrain_41_r | 8.29 | 0.170271053 |
| HS_from_MM_163_l | 5.4 | 0.170271053 |
| HSbrain_177_l | 3.34 | 0.170271053 |
| HSbrain_755_r | 2.58 | 0.170271053 |
| Hsd_47_r | 2.41 | 0.170271053 |
| HS_264_r | 2.41 | 0.170271053 |
| Hsd_from_Mmd_92_l | 25.04 | 0.176183859 |
| HS_from_MQbrain_1832_r | 7.61 | 0.176183859 |
| Hsd_from_Mmd_212_r | 6.76 | 0.176183859 |
| HS_from_MQbrain_520_l | 5.39 | 0.176183859 |
| HS_from_MQbrain_1565_l | 4.63 | 0.176183859 |
| Hsd_from_Mmd_412_r | 3.21 | 0.176183859 |
| HSbrain_491_l | 3.17 | 0.176183859 |
| HSbrain_151_r | 2.12 | 0.176183859 |
| Hsd_27_r | 2.1 | 0.176183859 |
| HS_from_MQbrain_1497_l | 2.09 | 0.176183859 |
| HS_from_PT_161_l | 23.16 | 0.182758186 |
| HS_from_MQbrain_1219_r | 20.97 | 0.182758186 |
| HS_from_MQbrain_635_l | 20.97 | 0.182758186 |
| HS_from_MQbrain_736_r | 15.8 | 0.182758186 |
| HS_from_MQbrain_559_r | 10.46 | 0.182758186 |
| HSbrain_456_r | 4.65 | 0.182758186 |
| HS_from_MQbrain_1883_l | 3.79 | 0.182758186 |
| HS_from_MQbrain_1137_l | 2.89 | 0.182758186 |
| Hsd_from_Mmd_68_l | 2.5 | 0.182758186 |
| HS_from_MQbrain_999_r | 84.53 | 0.188867139 |
| HSbrain_403_l | 20.91 | 0.188867139 |
| HS_from_MQbrain_524_l | 8.94 | 0.188867139 |
| HSbrain_153_r | 8.32 | 0.188867139 |
| HS_from_MQbrain_178_r | 8.01 | 0.188867139 |
| HS_from_MQbrain_1353_l | 5.24 | 0.188867139 |
| Hsd_from_Mmd_120_l | 3.25 | 0.188867139 |
| HS_from_MM_331_r | 2.57 | 0.188867139 |
| HS_from_MQbrain_436_r | 2.14 | 0.188867139 |
| HS_203_l | 52.23 | 0.19639408 |
| HSbrain_658_l | 17.81 | 0.19639408 |
| Hsd_from_Mmd_399_r | 11.23 | 0.19639408 |
| HS_from_MQbrain_1402_r | 9.06 | 0.19639408 |
| HSbrain_690_r | 5.57 | 0.19639408 |
| HS_from_MQbrain_1671_l | 2.75 | 0.19639408 |
| HSbrain_295_r | 2.25 | 0.19639408 |
| HSbrain_143_r | 2.01 | 0.19639408 |

¹Cut off Signal/background > 2
²Cut off adj p < 0.2

TABLE 15a
Small RNAs recovered from blood and differentially expressed in
adenocarcinoma vs normal tissue: hairpin arm with highest expression

| | 1^{st} arm | carcinoma vs |
|---|---|---|
| Systematic name | signal/background¹ | control (adj p)² |
| HS_from_MM_334_l | 71.92 | 0.016433541 |
| HSbrain_377_r | 3.94 | 0.019274533 |
| HSbrain_617_l | 106.18 | 0.022921248 |
| HS_from_MM_178_r | 3.95 | 0.030988376 |
| HS_from_MQbrain_1056_l | 5.37 | 0.031989408 |
| HS_from_MQbrain_1823_r | 6.11 | 0.0342406 |
| Hsd_102_l | 51.2 | 0.035303066 |
| HS_from_PT_190_r | 545.93 | 0.045228199 |
| HSbrain_171_l | 2560.96 | 0.067337504 |
| HS_from_MQbrain_343_l | 224.21 | 0.07566513 |
| HS_from_MQbrain_1169_l | 27.22 | 0.084904114 |
| HSbrain_12_l | 6.74 | 0.086503988 |
| HS_from_MQbrain_350_l | 188.59 | 0.086503988 |
| HSbrain_597_r | 342.91 | 0.086503988 |
| HSbrain_629_l | 1754.85 | 0.086503988 |
| HS_255_l | 144.06 | 0.090694253 |
| HS_from_MQbrain_1128_l | 33.94 | 0.090694253 |
| HS_from_MQbrain_349_l | 214.68 | 0.090694253 |
| HS_from_MQbrain_55_l | 2.66 | 0.090694253 |
| HS_from_MQbrain_2380_r | 100.43 | 0.090694253 |
| Hsd_from_Mmd_328_r | 3.25 | 0.090694253 |
| HS_from_PT_340_l | 74.7 | 0.090694253 |
| HSbrain_494_l | 2.84 | 0.104922786 |
| HSbrain_177_r | 9.75 | 0.104922786 |
| HSbrain_483_l | 33.11 | 0.110995143 |
| HS_from_MQbrain_128_r | 205.98 | 0.110995143 |
| HS_from_MM_309_r | 39.96 | 0.120015634 |
| HSbrain_709_l | 1509.68 | 0.120015634 |
| HS_141_l | 105.74 | 0.120843678 |
| HS_217_l | 34.38 | 0.120843678 |
| HS_from_MQbrain_1657_r | 10.62 | 0.120843678 |
| HS_from_PT_257_r | 2.92 | 0.120843678 |
| HS_from_MQbrain_930_r | 8.71 | 0.120843678 |
| HS_from_MQbrain_769_r | 3.62 | 0.120843678 |
| HS_from_MQbrain_572_l | 7.03 | 0.126655157 |
| HS_from_MQbrain_1624_l | 1452.35 | 0.149145996 |
| HSbrain_1_l | 471.82 | 0.149145996 |
| HSbrain_158_l | 43.6 | 0.149145996 |
| HSbrain_801_r | 81.51 | 0.149145996 |
| Hsd_111_r | 92.66 | 0.149145996 |
| Hsd_18_l | 551.25 | 0.159103307 |
| HSbrain_261_l | 4.72 | 0.159103307 |
| HSbrain_458_l | 14.44 | 0.166408872 |
| HS_from_MQbrain_1459_l | 154.95 | 0.166408872 |
| HS_from_MM_339_r | 2501.25 | 0.179914563 |
| HS_304_r | 442.22 | 0.179914563 |
| Hsd_from_Mmd_298_r | 2.19 | 0.19263196 |
| HS_from_MQbrain_1513_r | 11.02 | 0.19263196 |

¹Cut off Signal/background > 2
²Cut off adj p < 0.2

TABLE 15b
Small RNAs recovered from blood and differentially expressed in
adenocarcinoma vs normal tissue: hairpin arm with lowest expression

| | 2^{nd} arm | carcinoma vs |
|---|---|---|
| Systematic name | signal/background¹ | control (adj p)² |
| HS_from_MQbrain_1393_r | 240.51 | 0.016433541 |
| HS_215_r | 6.03 | 0.016433541 |
| HS_291_r | 4.17 | 0.035303066 |
| HS_21_r | 99.11 | 0.099652833 |
| HS_from_MQbrain_1103_l | 2.46 | 0.099652833 |
| HS_from_MM_289_l | 5.4 | 0.104922786 |
| HS_from_PT_26_r | 5.11 | 0.110995143 |
| HS_156_l | 7.17 | 0.120015634 |
| HS_from_MQbrain_1696_l | 43.62 | 0.126655157 |
| HSbrain_299_r | 32.14 | 0.159103307 |

¹ Cut off Signal/background > 2
² Cut off adj p < 0.2

TABLE 16a
Small RNAs recovered from blood and differentially expressed in
adenocarcinoma vs adenoma: hairpin arm with highest expression

| | 1^{st} arm | carcinoma vs |
|---|---|---|
| Systematic name | (signal/background)¹ | adenoma (adj p)² |
| HS_from_MQbrain_1391_l | 4.14 | 0.012664553 |
| HS_169_l | 138.82 | 0.014416465 |
| HS_from_MQbrain_1334_r | 10.69 | 0.015599346 |
| HS_299_l | 29.47 | 0.016786613 |
| HS_from_MQbrain_1082_l | 744.36 | 0.018319912 |
| HS_from_MQbrain_1522_r | 7.73 | 0.020545593 |
| HSbrain_306_r | 2986.89 | 0.023919533 |
| HS_from_MQbrain_629_l | 237.88 | 0.026776445 |
| HS_from_MQbrain_1456_l | 251.46 | 0.028202169 |
| HS_from_MQbrain_828_l | 1227.9 | 0.032034428 |
| HSbrain_384_l | 3.79 | 0.034662803 |
| HSbrain_1425_l | 1793.99 | 0.035941334 |
| HS_from_MQbrain_1750_r | 2658.65 | 0.037874034 |
| HS_from_MQbrain_984_l | 6.6 | 0.039250091 |
| HS_from_MQbrain_43_r | 4.54 | 0.039250091 |
| HS_141_l | 105.74 | 0.044599949 |
| HS_from_MQbrain_686_r | 2145.46 | 0.044599949 |
| HS_218_r | 2191.38 | 0.045945376 |
| HS_217_l | 34.38 | 0.050135128 |
| HSbrain_226_r | 1502.51 | 0.050135128 |
| HSbrain_494_l | 2.84 | 0.053747925 |
| HS_from_MQbrain_1340_l | 248.35 | 0.058254254 |
| HSbrain_337_l | 18.24 | 0.059897432 |
| HSbrain_25_r | 4.72 | 0.062381992 |
| HS_from_MQbrain_330_r | 3085.23 | 0.0659723 |
| HS_261_r | 142.25 | 0.068590471 |
| Hsd_from_Mmd_391_l | 3.15 | 0.070590475 |
| HS_from_MQbrain_1832_l | 2323.53 | 0.071451516 |
| Hsd_from_Mmd_237_l | 2452.36 | 0.071451516 |
| HS_from_MQbrain_93_r | 1882.36 | 0.071451516 |
| HSbrain_2_l | 84.89 | 0.072275086 |
| HSbrain_52_r | 24.74 | 0.072275086 |
| HSbrain_182_r | 2216.91 | 0.072275086 |
| HS_from_MQbrain_869_l | 49.56 | 0.072654718 |
| HS_from_MQbrain_74_r | 1469.53 | 0.074901054 |
| HSbrain_419_l | 1039 | 0.074901054 |
| HS_from_MQbrain_470_l | 136.89 | 0.075028175 |
| HS_from_MM_103_l | 2974.49 | 0.075028175 |
| HS_219_l | 2255.05 | 0.075028175 |
| HS_from_MQbrain_1001_r | 1683.09 | 0.075028175 |
| Hsd_from_Mmd_527_r | 11.56 | 0.07641095 |
| Hsd_from_Mmd_189_l | 233.41 | 0.07641095 |
| HS_from_MQbrain_1462_l | 33.63 | 0.07715065 |
| HSbrain_253_l | 67.08 | 0.07715065 |
| HS_118_l | 634.82 | 0.07715065 |
| HS_119_l | 2088.91 | 0.07715065 |
| HSbrain_204_r | 1075.46 | 0.07715065 |
| HSbrain_442_r | 5.68 | 0.07965977 |
| HSbrain_361_l | 2360.92 | 0.080676612 |
| HSbrain_328_l | 3271.08 | 0.08237725 |
| HS_from_PT_27_r | 1359.52 | 0.08237725 |
| HS_from_MQbrain_1569_l | 171.53 | 0.083833911 |
| HSbrain_6_r | 2079.02 | 0.084885584 |
| HS_from_MQbrain_1443_r | 361.33 | 0.084885584 |
| Hsd_from_Mmd_261_l | 2305.13 | 0.084885584 |
| HSbrain_389_l | 15.54 | 0.084885584 |
| HS_from_MQbrain_1675_r | 791.8 | 0.084885584 |
| HSbrain_95_l | 464.25 | 0.084885584 |
| HS_from_MQbrain_1623_r | 992.29 | 0.084885584 |
| HS_from_MQbrain_1494_r | 8.2 | 0.084885584 |
| HS_from_MQbrain_965_l | 243.53 | 0.085697388 |
| Hsd_101_l | 127.99 | 0.085697388 |
| HSbrain_764_r | 42.39 | 0.085697388 |
| HS_from_MQbrain_928_r | 1544.69 | 0.085697388 |
| HS_from_MQbrain_1355_r | 2362.21 | 0.089491072 |
| HSbrain_695_l | 10.34 | 0.089491072 |
| HSbrain_540_r | 2.58 | 0.091337111 |
| HS_from_MM_219_l | 151.42 | 0.091337111 |
| HS_from_MQbrain_1771_r | 1664.95 | 0.091337111 |
| HS_from_MQbrain_631_l | 1729.43 | 0.091337111 |
| HS_from_MQbrain_765_l | 2695.54 | 0.091337111 |
| HS_from_MQbrain_392_l | 1903.25 | 0.091337111 |
| HSbrain_594_l | 1061.87 | 0.091337111 |
| HS_from_MQbrain_131_r | 447.53 | 0.092780591 |
| HS_from_MQbrain_634_l | 7.38 | 0.092780591 |
| HS_from_MQbrain_191_l | 276.03 | 0.094556199 |
| Hsd_from_Mmd_330_l | 1411.51 | 0.094556199 |
| HSbrain_591_l | 1813.41 | 0.094556199 |
| HSbrain_123_l | 2450.67 | 0.096132958 |
| HS_from_MQbrain_1793_r | 4.95 | 0.096132958 |
| HS_from_MQbrain_1183_l | 112.54 | 0.098301345 |
| HSbrain_316_l | 1216.28 | 0.098301345 |
| HS_from_MQbrain_1031_r | 987.03 | 0.099488281 |
| Hsd_70_l | 685 | 0.101009439 |
| HSbrain_703_l | 1943.04 | 0.101009439 |
| HSbrain_14_r | 47.92 | 0.101009439 |
| HS_from_MQbrain_1827_l | 2658.72 | 0.101009439 |
| HS_from_MQbrain_628_l | 20.61 | 0.102601153 |
| HS_from_MQbrain_1374_l | 242.85 | 0.104515233 |
| HS_from_MQbrain_433_l | 49.52 | 0.104515233 |
| HS_from_MQbrain_722_r | 36.55 | 0.104515233 |
| HS_from_MQbrain_62_l | 2.3 | 0.104515233 |
| HS_from_MQbrain_22_r | 1898.63 | 0.105993846 |
| HS_from_MQbrain_534_r | 1775.11 | 0.105993846 |
| HS_from_MQbrain_951_l | 216.15 | 0.107794024 |
| HS_from_MQbrain_574_l | 439.75 | 0.107794024 |
| HS_from_MQbrain_921_l | 1527.75 | 0.107794024 |
| HS_from_MQbrain_996_l | 2730.34 | 0.107794024 |
| HSbrain_677_l | 2284.34 | 0.11066006 |
| Hsd_from_Mmd_52_r | 145.37 | 0.11066006 |
| HS_from_MM_86_l | 3.47 | 0.11066006 |
| Hsd_from_Mmd_266_r | 148.01 | 0.11066006 |
| HSbrain_426_r | 1715.96 | 0.113095726 |
| HSbrain_50_l | 1128.11 | 0.113095726 |
| HSbrain_588_l | 2412.89 | 0.113095726 |
| HSbrain_440_l | 2827.75 | 0.113095726 |
| HS_from_MQbrain_777_r | 5.15 | 0.113095726 |
| HS_from_MQbrain_29_l | 1136.64 | 0.113095726 |
| HS_from_MQbrain_56_l | 125.18 | 0.114595796 |
| HS_102_l | 2248.72 | 0.114595796 |
| HS_from_MQbrain_1164_r | 1604.05 | 0.114595796 |
| HS_71_l | 28.7 | 0.115928328 |
| HS_from_MQbrain_1637_r | 961.67 | 0.115928328 |
| HS_from_MQbrain_609_l | 1532.45 | 0.115928328 |
| HS_from_MQbrain_501_l | 1907.55 | 0.115928328 |
| HS_from_MQbrain_354_r | 14.17 | 0.117828853 |
| HS_246_l | 518 | 0.117828853 |
| HS_from_MM_208_r | 1993.74 | 0.117828853 |
| HS_from_MQbrain_974_l | 1938.97 | 0.119559457 |
| HSbrain_708_l | 2404.97 | 0.119559457 |
| HS_from_PT_60_l | 2122.55 | 0.119559457 |
| HSbrain_606_l | 993.78 | 0.119559457 |
| HSbrain_305_r | 2302.79 | 0.121604253 |
| HS_from_MQbrain_1632_l | 3014.17 | 0.121604253 |
| HS_from_MQbrain_1075_l | 2494.05 | 0.12300785 |
| HSbrain_402_l | 2283.48 | 0.12300785 |
| HS_from_MQbrain_266_l | 878.84 | 0.12300785 |
| HS_from_MQbrain_1261_r | 2483.06 | 0.12300785 |
| HS_from_MQbrain_340_r | 387 | 0.12300785 |
| HS_from_MQbrain_1097_r | 22.37 | 0.124263194 |
| HS_from_MQbrain_210_r | 1848.78 | 0.124263194 |
| HS_from_MQbrain_681_l | 2.58 | 0.124263194 |
| HS_from_MQbrain_314_l | 3.26 | 0.126757752 |
| HSbrain_771_l | 1417.26 | 0.126757752 |
| HS_from_MQbrain_1102_l | 2428.54 | 0.129555956 |
| HSbrain_393_r | 1557.67 | 0.129555956 |
| HS_from_MQbrain_1413_l | 2113.32 | 0.129555956 |
| HS_from_MQbrain_800_l | 28.04 | 0.129555956 |
| HSbrain_783_l | 2871.64 | 0.130803986 |
| Hsd_from_Mmd_94_l | 10 | 0.130803986 |
| HS_from_PT_313_l | 19.26 | 0.130803986 |
| HS_from_MQbrain_672_l | 2227.7 | 0.130803986 |
| HS_from_MQbrain_216_l | 1590.82 | 0.130803986 |
| HSbrain_380_r | 28.02 | 0.130803986 |
| HS_from_MQbrain_1100_l | 37.05 | 0.132596379 |
| HS_from_MQbrain_715_l | 2873.52 | 0.132596379 |
| HS_from_MQbrain_868_l | 251.95 | 0.132596379 |
| HSbrain_691_l | 1101.52 | 0.132596379 |
| HS_101_l | 2303.1 | 0.132596379 |
| HSbrain_243_l | 593.95 | 0.134691663 |
| HS_62_l | 8.32 | 0.134691663 |
| Hsd_42_r | 755.65 | 0.134691663 |
| Hsd_7_r | 7.55 | 0.134691663 |
| HSbrain_484_l | 158.28 | 0.134691663 |
| HS_from_PT_44_l | 2616.23 | 0.136637202 |
| HS_from_MQbrain_1043_r | 1537.86 | 0.138217661 |
| HS_from_MQbrain_315_l | 248.59 | 0.138217661 |
| HS_from_MQbrain_70_l | 2632.56 | 0.138217661 |
| HS_from_MQbrain_1836_l | 5.19 | 0.138217661 |
| HS_from_MQbrain_1824_l | 2661.19 | 0.138217661 |
| HSbrain_250_l | 2758.18 | 0.138217661 |
| Hsd_69_l | 1207.06 | 0.138217661 |
| HSbrain_420_r | 1374.92 | 0.139019426 |
| HS_from_MQbrain_1493_r | 472.59 | 0.139019426 |
| Hsd_13_l | 16.95 | 0.139019426 |
| HS_from_MQbrain_1756_l | 1749.72 | 0.139019426 |
| HS_277_l | 776.25 | 0.139019426 |
| HS_from_MQbrain_809_l | 21.62 | 0.139019426 |
| HS_from_MQbrain_886_r | 1484.58 | 0.139019426 |
| HS_from_MQbrain_90_r | 1911.93 | 0.139019426 |
| HS_from_MQbrain_399_l | 1395.01 | 0.139019426 |
| HS_98_r | 2382.13 | 0.141416815 |
| HS_from_MQbrain_845_r | 365.11 | 0.141416815 |
| HS_from_MQbrain_1331_r | 2711.38 | 0.141416815 |
| HS_39_r | 1944.78 | 0.141416815 |
| HS_from_MQbrain_824_r | 1660.01 | 0.14474535 |
| HSbrain_369_r | 197.24 | 0.14474535 |
| HS_from_MQbrain_914_l | 2107.69 | 0.14474535 |
| HS_from_MQbrain_1143_l | 566.08 | 0.14474535 |
| HSbrain_82_r | 10.7 | 0.14474535 |
| HS_from_MQbrain_1227_l | 228.29 | 0.145370181 |
| HSbrain_214_l | 5.03 | 0.145370181 |
| HS_175_l | 3.1 | 0.145370181 |
| HSbrain_716_l | 1552.16 | 0.145370181 |
| HS_from_MQbrain_1204_r | 2072.07 | 0.145370181 |
| HS_54_l | 705.25 | 0.145370181 |
| HS_from_MQbrain_1810_l | 923.33 | 0.145370181 |
| HSbrain_117_r | 181.24 | 0.145370181 |
| HS_from_MQbrain_535_r | 45.21 | 0.145370181 |
| HSbrain_620_l | 161.99 | 0.145370181 |
| HS_from_MQbrain_180_r | 1754.02 | 0.145370181 |
| HS_from_MM_199_l | 79.91 | 0.146325218 |
| HS_133_l | 2696.57 | 0.146325218 |
| Hsd_from_Mmd_246_r | 185.28 | 0.146325218 |
| HS_306_l | 22.09 | 0.146325218 |
| HSbrain_307_r | 2494.19 | 0.146325218 |
| HS_from_MQbrain_973_l | 140.65 | 0.146325218 |
| HS_from_MQbrain_1444_l | 2.03 | 0.148196723 |
| HS_from_MQbrain_671_r | 1023.79 | 0.148196723 |
| HS_from_MM_118_l | 5.89 | 0.148196723 |
| HSbrain_624_l | 1034.54 | 0.148196723 |
| HSbrain_627_r | 27.61 | 0.148196723 |
| HSbrain_647_r | 16.58 | 0.148196723 |
| HSbrain_177_r | 9.75 | 0.149149579 |
| HS_from_MQbrain_1773_l | 479.94 | 0.149149579 |
| HSbrain_198_l | 7.92 | 0.149149579 |
| HS_from_PT_134_l | 2880.24 | 0.149149579 |
| HS_from_MQbrain_1088_l | 162.85 | 0.149149579 |
| HS_139_l | 2103.33 | 0.149149579 |
| HS_from_MQbrain_817_l | 5.3 | 0.149149579 |
| HS_from_MQbrain_1419_r | 1654.34 | 0.149149579 |
| HS_58_l | 1112.12 | 0.149149579 |
| HSbrain_41_l | 264.89 | 0.152177614 |
| HSbrain_717_l | 2399.92 | 0.152177614 |
| HS_from_PT_20_r | 1411.59 | 0.152177614 |
| HS_204_r | 2.11 | 0.152177614 |
| HSbrain_304_l | 60.38 | 0.152177614 |
| HS_111_r | 2642.45 | 0.152177614 |
| Hsd_from_Mmd_509_l | 2.9 | 0.152177614 |
| HS_from_MQbrain_1128_l | 33.94 | 0.155479351 |
| HS_from_MQbrain_536_r | 1796.18 | 0.155479351 |
| HS_from_MQbrain_1433_r | 232.94 | 0.155479351 |
| HSbrain_578_r | 119.12 | 0.155479351 |
| HSbrain_652_l | 1052.64 | 0.15865589 |
| HSbrain_673_l | 2432.23 | 0.15865589 |
| HS_220_l | 2.15 | 0.15865589 |
| HSbrain_431_l | 2250.77 | 0.15865589 |
| HS_from_MQbrain_1168_l | 25.63 | 0.15865589 |
| HS_from_PT_254_r | 11.89 | 0.159708794 |
| Hsd_from_Mmd_480_r | 1038.29 | 0.159708794 |
| HS_from_MQbrain_604_l | 2116.19 | 0.159708794 |
| HSbrain_571_l | 5.09 | 0.159708794 |
| HS_from_MQbrain_529_r | 1669.81 | 0.159708794 |
| HS_167_r | 2325.07 | 0.159708794 |
| HSbrain_202_r | 646.85 | 0.159708794 |
| HS_288_l | 1661.49 | 0.161647438 |
| HSbrain_72_l | 1994.62 | 0.161647438 |
| HS_from_MQbrain_1839_l | 2242.94 | 0.161647438 |
| HS_from_MQbrain_1722_l | 121.03 | 0.161647438 |
| HS_from_MQbrain_587_l | 3.45 | 0.161647438 |
| HS_from_MQbrain_1877_r | 1438.74 | 0.161647438 |
| HSbrain_16_l | 6.68 | 0.161647438 |
| HS_from_MM_319_l | 107.03 | 0.16463875 |
| HS_208_r | 514.2 | 0.16463875 |
| HS_from_PT_41_l | 688.41 | 0.16463875 |
| HS_from_MQbrain_808_l | 23 | 0.16463875 |
| HS_from_MQbrain_1229_r | 1561.04 | 0.16463875 |
| HSbrain_220_l | 1272.15 | 0.16463875 |
| HSbrain_111_l | 5.04 | 0.16463875 |
| HS_from_MQbrain_1268_l | 6.35 | 0.167313007 |
| HS_from_MQbrain_2670_r | 19.49 | 0.167313007 |
| HSbrain_168_l | 636.93 | 0.167313007 |
| HS_from_MQbrain_699_r | 2022.54 | 0.167313007 |
| HS_from_MQbrain_1372_l | 983.18 | 0.167313007 |
| HSbrain_513_r | 778.63 | 0.167313007 |
| HS_from_MQbrain_1876_r | 2589.02 | 0.167313007 |
| HS_114_l | 715.1 | 0.170255758 |
| HSbrain_113_r | 77.28 | 0.170255758 |
| HS_from_MQbrain_1855_l | 1224.15 | 0.170255758 |
| HS_61_r | 2.57 | 0.170255758 |
| HS_from_MQbrain_496_r | 2625.7 | 0.170255758 |
| HSbrain_157_l | 60.78 | 0.173467438 |
| HSbrain_336_l | 4.14 | 0.173467438 |
| HSbrain_614_l | 2539.17 | 0.173467438 |
| HS_29_l | 950.02 | 0.173467438 |
| HS_from_MQbrain_57_l | 94.28 | 0.173467438 |
| HS_from_MQbrain_734_l | 2968.08 | 0.175200251 |
| HS_from_MQbrain_1777_l | 3386.15 | 0.175200251 |
| HS_from_MQbrain_437_l | 15.23 | 0.175200251 |
| HSbrain_643_l | 1935.82 | 0.175200251 |
| HS_from_PT_34_r | 2623.29 | 0.175200251 |
| HSbrain_687_l | 1178.66 | 0.175200251 |
| HS_from_MQbrain_1091_l | 2553.31 | 0.175200251 |
| HS_from_MQbrain_1708_l | 257.81 | 0.175200251 |
| HS_from_MQbrain_1463_l | 136.6 | 0.175200251 |
| HS_from_MQbrain_1485_r | 5.89 | 0.177405071 |
| HSbrain_1305_l | 237.71 | 0.177405071 |
| HSbrain_347_l | 1854.13 | 0.177405071 |
| HSbrain_794_l | 1200.68 | 0.177405071 |
| HS_from_MQbrain_1470_r | 2318.58 | 0.177405071 |
| Hsd_from_Mmd_167_r | 2610.41 | 0.177405071 |
| HSbrain_692_r | 2311.79 | 0.177405071 |
| HSbrain_690_l | 1719.12 | 0.177405071 |
| Hsd_from_Mmd_42_r | 30.99 | 0.177405071 |
| HS_266_r | 5.54 | 0.180260145 |
| HS_from_MQbrain_550_l | 2602.74 | 0.180260145 |
| HS_from_MQbrain_1631_r | 2710.28 | 0.180260145 |
| HSbrain_593_r | 1548.5 | 0.180260145 |
| HS_285_r | 925.13 | 0.180260145 |
| HSbrain_263_r | 37.25 | 0.180260145 |
| Hsd_from_Mmd_286_r | 4.4 | 0.180260145 |
| Hsd_from_Mmd_120_r | 1906.46 | 0.184337179 |
| HSbrain_621_l | 2344.81 | 0.184337179 |
| HSbrain_778_l | 91.71 | 0.184337179 |
| HS_243_l | 1837.67 | 0.184337179 |
| HS_138_l | 125.63 | 0.184337179 |
| HSbrain_1364_l | 1439.39 | 0.185815478 |
| HSbrain_32_l | 1943.03 | 0.185815478 |
| HS_77_l | 1287.06 | 0.185815478 |
| HS_from_MQbrain_1778_r | 2.54 | 0.185815478 |
| HS_303_r | 290.92 | 0.185815478 |
| HS_from_MQbrain_272_l | 1172.26 | 0.185815478 |
| HS_from_MQbrain_453_r | 3.84 | 0.185815478 |
| HSbrain_166_l | 2487.26 | 0.185815478 |
| HS_142_l | 1918.3 | 0.185815478 |
| HS_from_MQbrain_17_r | 39.73 | 0.185815478 |
| HSbrain_662_r | 2.36 | 0.185815478 |
| HS_from_MQbrain_940_r | 317.39 | 0.188706417 |
| HS_189_l | 103.99 | 0.188706417 |
| HS_from_MQbrain_745_r | 2408.79 | 0.188706417 |
| HS_from_MQbrain_36_r | 1728.75 | 0.188706417 |
| HSbrain_570_l | 1042.7 | 0.188706417 |
| HSbrain_497_l | 5.9 | 0.188706417 |
| HSbrain_141_r | 2820.17 | 0.188706417 |
| HS_from_MQbrain_691_r | 1239.61 | 0.188706417 |
| HS_50_l | 1776.71 | 0.188706417 |
| Hsd_62_l | 733.75 | 0.190550803 |
| HS_from_MQbrain_397_l | 2241.37 | 0.190550803 |
| HS_from_PT_238_l | 2054.08 | 0.190550803 |
| HS_from_MQbrain_97_r | 346.64 | 0.190550803 |
| HS_166_l | 2194.77 | 0.190550803 |
| HS_from_MQbrain_626_r | 2474.16 | 0.190550803 |
| HS_from_MQbrain_968_r | 1722.97 | 0.190550803 |
| HS_105_l | 2468.12 | 0.190550803 |
| HS_from_MM_80_l | 2105.14 | 0.190550803 |
| HSbrain_809_r | 1761.33 | 0.190550803 |
| Hsd_from_Mmd_298_r | 2.19 | 0.194339793 |
| HSbrain_259_r | 1597.52 | 0.194339793 |
| HSbrain_755_l | 2.82 | 0.194339793 |
| HS_from_MQbrain_1272_l | 1771.43 | 0.194339793 |
| HS_from_MQbrain_1421_l | 2558.01 | 0.194339793 |
| HS_from_PT_149_r | 2.84 | 0.194339793 |
| HS_from_MQbrain_467_l | 182.78 | 0.197457594 |
| HSbrain_210_l | 2938.2 | 0.197457594 |
| HS_from_MQbrain_635_r | 1594.99 | 0.197457594 |
| HS_from_MQbrain_1159_l | 2053.58 | 0.197457594 |
| HSbrain_479_l | 10.28 | 0.197457594 |
| HS_from_MQbrain_1514_r | 2.92 | 0.197457594 |
| HS_from_MM_63_l | 4.64 | 0.199721903 |
| HSbrain_482_l | 36.52 | 0.199721903 |
| Hsd_from_Mmd_478_l | 40.92 | 0.199721903 |
| HSbrain_388_r | 1236.45 | 0.199721903 |
| HSbrain_651_r | 2230.86 | 0.199721903 |
| HS_from_MQbrain_1357_l | 1967.07 | 0.199721903 |
| HS_from_MQbrain_1210_l | 1544.01 | 0.199721903 |

¹Cut off Signal/background > 2
²Cut off adj p < 0.2

TABLE 16a
Small RNAs recovered from blood and differentially expressed in
adenocarcinoma vs adenoma: hairpin arm with lowest expression

| | 2^{nd} arm | carcinoma vs |
|---|---|---|
| Systematic name | (signal/background)¹ | adenoma (adj p)² |
| HS_from_MQbrain_638_l | 2.57 | 0.00750624 |
| HSbrain_390_l | 3.48 | 0.012664553 |
| HS_8_r | 4.22 | 0.014224152 |
| HS_176_l | 2.96 | 0.014416465 |
| HSbrain_293_l | 2.65 | 0.015226536 |
| HS_from_MQbrain_63_r | 2.96 | 0.015599346 |
| HS_from_MQbrain_1137_l | 2.89 | 0.016904198 |
| HS_291_r | 4.17 | 0.020545593 |
| HS_157_r | 3.41 | 0.020545593 |
| HSbrain_217_r | 2.23 | 0.020545593 |
| HS_51_r | 2.98 | 0.024910624 |
| HSbrain_680_r | 8.65 | 0.031432755 |
| HSbrain_687_r | 7.98 | 0.034662803 |
| Hsd_70_r | 2.28 | 0.035037528 |
| HSbrain_351_r | 3.85 | 0.039250091 |
| Hsd_18_r | 2.34 | 0.039250091 |
| HSbrain_1325_r | 5.49 | 0.040852758 |
| HSbrain_669_r | 8.92 | 0.044599949 |
| HSbrain_703_r | 2.01 | 0.047341086 |
| HSbrain_304_r | 6.39 | 0.04754573 |
| HSbrain_177_l | 3.34 | 0.048205391 |
| HSbrain_122_l | 3.01 | 0.049767746 |
| HSbrain_187_r | 7.95 | 0.050135128 |
| HS_from_MQbrain_1565_l | 4.63 | 0.050135128 |
| HSbrain_673_r | 9.27 | 0.052756328 |
| HSbrain_684_r | 8.86 | 0.052756328 |
| HS_17_l | 4.22 | 0.053747925 |
| Hsd_from_Mmd_371_r | 2.09 | 0.058218978 |
| HSbrain_374_r | 4.56 | 0.058254254 |
| HS_from_MQbrain_346_l | 2.9 | 0.059897432 |
| Hsd_from_Mmd_413_r | 2.19 | 0.06455063 |
| HS_from_MQbrain_932_l | 13.46 | 0.068270026 |
| HS_from_MQbrain_108_r | 6.38 | 0.068590471 |
| HS_from_MQbrain_736_r | 15.8 | 0.071451516 |
| HSbrain_93_r | 9.48 | 0.072275086 |
| HS_from_MQbrain_1006_l | 3.57 | 0.072275086 |
| HS_200_r | 6.42 | 0.072654718 |
| HS_from_MQbrain_301_l | 4.97 | 0.072654718 |
| HSbrain_47_r | 9.58 | 0.075028175 |
| HS_from_MQbrain_154_r | 3.24 | 0.07715065 |
| HSbrain_319_l | 6.7 | 0.07965977 |
| Hsd_42_l | 3.47 | 0.083833911 |
| HS_from_MQbrain_634_r | 2.4 | 0.083833911 |
| HS_from_MQbrain_824_l | 17.99 | 0.084885584 |
| HS_54_r | 2.68 | 0.084885584 |
| HS_from_MQbrain_534_l | 4.92 | 0.085697388 |
| HSbrain_512_l | 4.96 | 0.089491072 |
| Hsd_27_r | 2.1 | 0.089491072 |
| HS_from_MQbrain_1264_l | 4.83 | 0.092780591 |
| HS_14_r | 2.37 | 0.092780591 |
| HS_from_MQbrain_1771_l | 115.56 | 0.098301345 |
| HSbrain_553_r | 3.4 | 0.098301345 |
| HS_from_MQbrain_129_r | 6.71 | 0.102601153 |
| HSbrain_661_l | 2.82 | 0.102601153 |
| HS_from_MQbrain_1116_r | 14.63 | 0.104515233 |
| Hsd_from_Mmd_405_l | 2.88 | 0.104515233 |
| HS_21_r | 99.11 | 0.107794024 |
| HS_from_MQbrain_1339_r | 2.71 | 0.11066006 |
| Hsd_12_r | 4.6 | 0.113095726 |
| HSbrain_677_r | 11.53 | 0.114595796 |
| HS_38_r | 10.14 | 0.114595796 |
| HSbrain_621_r | 2.02 | 0.114595796 |
| HSbrain_795_r | 2.01 | 0.114595796 |
| HS_from_MM_331_r | 2.57 | 0.115928328 |
| HS_from_MM_260_l | 2.24 | 0.117828853 |
| HSbrain_441_r | 4.99 | 0.119559457 |
| HSbrain_1437_r | 4.06 | 0.119559457 |
| HS_from_MQbrain_648_r | 84.85 | 0.121604253 |
| HS_154_r | 2.89 | 0.121604253 |
| HS_from_MQbrain_760_l | 13.31 | 0.12300785 |
| HS_from_MQbrain_1633_r | 9.89 | 0.124263194 |
| HSbrain_393_l | 6.32 | 0.124263194 |
| HS_from_MQbrain_268_r | 2.07 | 0.124263194 |
| HS_from_MQbrain_1675_l | 2.27 | 0.126757752 |
| HS_from_MQbrain_453_l | 3.29 | 0.129555956 |
| HSbrain_776_l | 156.06 | 0.130803986 |
| HS_from_MQbrain_37_r | 13.27 | 0.130803986 |
| Hsd_from_Mmd_219_r | 5.96 | 0.130803986 |
| HS_from_MQbrain_1520_l | 9.18 | 0.132596379 |
| HS_from_MQbrain_382_l | 2.37 | 0.132596379 |
| HS_from_MQbrain_1549_l | 2.35 | 0.134691663 |
| HS_from_MQbrain_281_r | 11.13 | 0.136637202 |
| HS_from_PT_95_l | 8.24 | 0.136637202 |
| HS_from_MQbrain_178_r | 8.01 | 0.139019426 |
| HSbrain_1416_r | 5.33 | 0.139019426 |
| HS_from_MQbrain_332_l | 5.24 | 0.141416815 |
| HS_from_PT_283_r | 7.34 | 0.145370181 |
| HSbrain_139_r | 3.45 | 0.145370181 |
| HS_from_MQbrain_64_l | 2.57 | 0.145370181 |
| HS_from_MQbrain_606_r | 51.46 | 0.146325218 |
| HS_from_MQbrain_666_l | 37.07 | 0.149149579 |
| HS_from_MQbrain_629_r | 4.6 | 0.155479351 |
| HS_from_MQbrain_1601_r | 343.75 | 0.15865589 |
| HS_from_MQbrain_936_l | 3.59 | 0.15865589 |
| HSbrain_218_l | 33.26 | 0.161647438 |
| HS_from_MQbrain_635_l | 20.97 | 0.161647438 |
| HS_from_PT_338_l | 2.54 | 0.167313007 |
| HS_from_MQbrain_401_l | 2.38 | 0.167313007 |
| HS_from_MQbrain_1627_r | 19.73 | 0.170255758 |
| HSbrain_626_r | 13.91 | 0.170255758 |
| HS_from_MQbrain_560_r | 9.17 | 0.173467438 |
| HS_from_MQbrain_85_l | 89.3 | 0.175200251 |
| HS_from_MQbrain_1752_l | 3.64 | 0.175200251 |
| HS_from_MQbrain_386_r | 337.53 | 0.177405071 |
| HSbrain_1414_r | 2.58 | 0.177405071 |
| Hsd_84_r | 4.61 | 0.184337179 |
| HS_182_r | 2.36 | 0.184337179 |
| HS_from_MQbrain_1394_l | 617.11 | 0.185815478 |
| HS_from_MQbrain_2700_r | 3.75 | 0.185815478 |
| HS_from_MQbrain_500_r | 7.18 | 0.197457594 |
| HS_from_MQbrain_968_l | 4.35 | 0.197457594 |
| HSbrain_190_r | 3.15 | 0.197457594 |
| HSbrain_755_r | 2.58 | 0.197457594 |
| HS_from_MQbrain_1100_r | 10.88 | 0.199721903 |
| HS_from_MQbrain_1415_r | 4.64 | 0.199721903 |
| HS_from_MQbrain_1747_l | 3.37 | 0.199721903 |

¹Cut off Signal/background > 2
²Cut off adj p < 0.2

TABLE 17
Summary of small RNAs detectable in faeces and blood and
differentially expressed in the different tissue categories

| Analysis | Sample | Reference | Result |
|---|---|---|---|
| Universal miRNAs | Carcinoma | Control | 59 miRNA recovered from |
| Table 10 | Faeces | | faeces and differentially |
| | | | expressed in |
| | | | adenocarcinoma vs control |
| Universal miRNAs | Adenoma | Control | 857 miRNA recovered from |
| Table 9 | Faeces | | faeces and differentially |
| | | | expressed in adenoma vs |
| | | | control |
| Universal miRNAs | Carcinoma | Adenoma | 461 miRNA recovered from |
| Table 11 | Faeces | | faeces and differentially |
| | | | expressed in |
| | | | adenocarcinoma vs |
| | | | adenoma |
| Universal miRNAs | Carcinoma | Control | 58 miRNA recovered from |
| Table 15 | Blood | | blood and differentially |
| | | | expressed in |
| | | | adenocarcinoma vs control |
| Universal miRNAs | Adenoma | Control | 842 miRNA recovered from |
| Table14 | Blood | | blood and differentially |
| | | | expressed in adenoma vs |
| | | | control |
| Universal miRNAs | Carcinoma | Adenoma | 461 miRNA recovered from |
| Table 16 | Blood | | blood and differentially |
| | | | expressed in |
| | | | adenocarcinoma vs |
| | | | adenoma |

### Presence of Classifying Small RNAs in Blood

Lengthy table referenced here
US20130190379A1-20130725-T00001
Please refer to the end of the specification for access instructions.

Lengthy table referenced here
US20130190379A1-20130725-T00002
Please refer to the end of the specification for access instructions.

Lengthy table referenced here
US20130190379A1-20130725-T00003
Please refer to the end of the specification for access instructions.

Lengthy table referenced here
US20130190379A1-20130725-T00004
Please refer to the end of the specification for access instructions.

Lengthy table referenced here
US20130190379A1-20130725-T00005
Please refer to the end of the specification for access instructions.

Lengthy table referenced here
US20130190379A1-20130725-T00006
Please refer to the end of the specification for access instructions.

Lengthy table referenced here
US20130190379A1-20130725-T00007
Please refer to the end of the specification for access instructions.

Lengthy table referenced here
US20130190379A1-20130725-T00008
Please refer to the end of the specification for access instructions.

Lengthy table referenced here
US20130190379A1-20130725-T00009
Please refer to the end of the specification for access instructions.

Lengthy table referenced here
US20130190379A1-20130725-T00010
Please refer to the end of the specification for access instructions.

Lengthy table referenced here
US20130190379A1-20130725-T00011
Please refer to the end of the specification for access instructions.

Lengthy table referenced here
US20130190379A1-20130725-T00012
Please refer to the end of the specification for access instructions.

Lengthy table referenced here
US20130190379A1-20130725-T00013
Please refer to the end of the specification for access instructions.

Lengthy table referenced here
US20130190379A1-20130725-T00014
Please refer to the end of the specification for access instructions.

Lengthy table referenced here
US20130190379A1-20130725-T00015
Please refer to the end of the specification for access instructions.

Lengthy table referenced here
US20130190379A1-20130725-T00016
Please refer to the end of the specification for access instructions.

Lengthy table referenced here
US20130190379A1-20130725-T00017
Please refer to the end of the specification for access instructions.

Lengthy table referenced here
US20130190379A1-20130725-T00018
Please refer to the end of the specification for access instructions.

Lengthy table referenced here
US20130190379A1-20130725-T00019
Please refer to the end of the specification for access instructions.

Lengthy table referenced here
US20130190379A1-20130725-T00020
Please refer to the end of the specification for access instructions.

Lengthy table referenced here
US20130190379A1-20130725-T00021
Please refer to the end of the specification for access instructions.

Lengthy table referenced here
US20130190379A1-20130725-T00022
Please refer to the end of the specification for access instructions.

Lengthy table referenced here
US20130190379A1-20130725-T00023
Please refer to the end of the specification for access instructions.

Lengthy table referenced here
US20130190379A1-20130725-T00024
Please refer to the end of the specification for access instructions.

Lengthy table referenced here
US20130190379A1-20130725-T00025
Please refer to the end of the specification for access instructions.

Lengthy table referenced here
US20130190379A1-20130725-T00026
Please refer to the end of the specification for access instructions.

Lengthy table referenced here
US20130190379A1-20130725-T00027
Please refer to the end of the specification for access instructions.

Lengthy table referenced here
US20130190379A1-20130725-T00028
Please refer to the end of the specification for access instructions.

Lengthy table referenced here
US20130190379A1-20130725-T00029
Please refer to the end of the specification for access instructions.

Lengthy table referenced here
US20130190379A1-20130725-T00030
Please refer to the end of the specification for access instructions.

LENGTHY TABLES
The patent application contains a lengthy table section. A copy of the table is available in electronic form from the USPTO web site (http://seqdata.uspto.gov/?pageRequest=docDetail&DocID=US20130190379A1). An electronic copy of the table will also be available from the USPTO upon request and payment of the fee set forth in 37 CFR 1.19(b)(3).

1. A collection of probes or primers for detecting each of the nucleic acid molecules of table 25a, table 5a and/or 6a.

2. A collection of probes or primers according to claim 1, comprising probes or primers for detecting each of the nucleic acid molecules of table 25a and table 6a.

3. A collection of probes or primers according to claim 2, comprising probes or primers for detecting each of the nucleic acid molecules of table 25a, table 5a and table 6a.

4. A collection of probes or primers for detecting each of the nucleic acid molecules of according to claim 1-3 for use in discriminating between normal colorectal tissue, colorectal adenoma and colorectal adenocarcinoma.

5. A collection of probes or primers for detecting each of the nucleic acid molecules of table 23 and/or table 24.

6. A method for selecting probes that discriminate between at least two types of samples comprising nucleic acid from cells said method comprising
hybridizing nucleic acid from a representative number of samples of said at least two types of samples to a collection of probes according to any one of claims 1-5,
quantifying hybridization of said probes with nucleic acid in said samples, and
selecting a subset of probes from said collection of probes, that discriminates between said at least two types of samples.

7. A method according to claim 6, wherein said cells are human cells.

8. A method according to claim 6 or claim 7, wherein at least one of said at least two types of samples comprises nucleic acid from tumor cells.

9. A method according to claim 8, wherein said tumor cells are colon tumor cells.

10. A method according to any one of claims 6-9, wherein at least one of said at least two types of samples comprises nucleic acid from faeces or blood.

11. A method according to any one of claims 6-10, wherein at least one of said at least two types of samples is a sample of normal cells, preferably normal colon epithelial cells.

12. A subset of probes or primers from a collection of probes or primers according to any one of claims 1-5, obtainable by a method according to any one of claims 6-11.

13. A subset of probes or primers according to claim 12, comprising probes or primers for at least 10 of the nucleic acid identified in table 23 or table 24.

14. A subset of probes or primers according to claim 12, comprising probes or primers for each of the nucleic acids identified in table 2a, table 3a, table 4a, table 5a, table 6a, table 7a, table 9a, table 9b, table 10a, table 10b, table 11a, table 11b, table 12a, table 12b, table 12c, table 14a, table 14b, table 15a, table 15b, table 16a, table 16b, table 18a, table 18b, table 18c, table 19c, table 19d, table 20, table 25a, table 26, table 27, table 28a, table 28b, table 29a, table 29b, table 30, table 31a, table 31b, table 32a, table 32b, table 33, table 34 and/or table 35.

15. A subset of probes or primers from the collection of probes or primers according to any one of claims 1-5, comprising probes or primers for each of the nucleic acids identified in table 8 or table 13.

16. A method for determining whether a sample comprises nucleic acid from a particular type of cells, said method comprising detecting and quantifying the level of the nucleic acid molecules identified in table 1, table 2a, table 3a, table 4a, table 5a, table 6a, table 7a, table 9a, table 9b, table 10a, table 10b, table 11a, table 11b, table 12a, table 12b, table 12c, table 14a, table 14b, table 15a, table 15b, table 16a, table 16b, table 18a, table 18b, table 18c, table 19c, table 19d, table 20, table 25a, table 26, table 27, table 28a, table 28b, table 29a, table 29b, table 30, table 31a, table 31b, table 32a, table 32b, table 33, table 34 and/or table 35 in said sample, and comparing the result of said quantification with a reference.

17. A method for determining whether a sample comprises nucleic acid of a particular type of cells, said method comprising hybridising nucleic acid from said sample to a collection of probes as identified in table 1, table 2a, table 3a, table 4a, table 5a, table 6a, table 7a, table 9a, table 9b, table 10a, table 10b, table 11a, table 11b, table 12a, table 12b, table 12c, table 14a, table 14b, table 15a, table 15b, table 16a, table 16b, table 18a, table 18b, table 18c, table 19c, table 19d, table 20 table 21, table 25a, table 26, table 27, table 28a, table 28b, table 29a, table 29b, table 30, table 31a, table 31b, table 32a, table 32b, table 33, table 34 and/or table 35, quantifying hybridization of said probes with nucleic acid in said sample, and comparing the result of said quantification with a reference.

18. A method according to claim 16 or claim 17, wherein said cells are tumor cells or suspected of being tumor cells.

19. A method according to any one of claims 16-18, wherein said cells are colon cells.

20. A method for determining whether a sample comprises nucleic acid from colorectal cells said method comprising detecting and quantifying the level of the nucleic acid molecules identified in table 1 in said sample, and comparing the result of said quantification with a reference.

21. A method for determining whether a sample comprises nucleic acid from colorectal cells said method comprising hybridising nucleic acid from said sample to a collection of probes as identified in table 1, quantifying hybridization of said probes with nucleic acid in said sample, and comparing the result of said quantification with a reference.

22. A method for determining whether a sample comprises nucleic acid from adenoma cells said method comprising detecting and quantifying the level of the nucleic acid molecules identified in table 2a, table 4a, table 5a, table 7a, table 9a, table 9b, table 11a, table 11b, table 12a, table 12c, table 14a, table 14b, table 16a, table 16b, table 18a, table 18c, table 19c, table 19d table 20, table 25a, table 26, table 27, table 34 and/or table 35 in said sample, and comparing the result of said quantification with a reference.

23. A method for determining whether a sample comprises nucleic acid from adenoma cells said method comprising hybridising nucleic acid from said sample to a collection of probes as identified in table 2a, table 4a, table 5a, table 7a, table 9a, table 9b, table 11a, table 11b, table 12a, table 12c, table 14a, table 14b, table 16a, table 16b, table 18a, table 18c, table 19c, table 19d, table 20 table 21, table 25a, table 26, table 27, table 34 and/or table 35, quantifying hybridization of said probes with nucleic acid in said sample, and comparing the result of said quantification with a reference.

24. A method according to claim 22 or claim 23, wherein said reference is a quantification of a hybridization of a sample comprising nucleic acid from adenocarcinoma cells or from normal cells, with said collection of probes.

25. A method for determining whether a sample comprises nucleic acid from adenocarcinoma cells said method comprising detecting and quantifying the level of the nucleic acid molecules identified in table 3a, table 4a, table 6a, table 7a, table 10a, table 10b, table 11a, table 11b, table 12b, table 12c, table 15a, table 15b, table 16a, table 16b, table 18b, table 18c, table 19c, table 19d, table 20, table 25a, table 26, table 27, table 28a, table 28b, table 29a, table 29b, table 30, table 31a, table 31b, table 32a, table 32b, table 33, table 34 and/or table 35 in said sample, and comparing the result of said quantification with a reference.

26. A method for determining whether a sample comprises nucleic acid from adenocarcinoma cells said method comprising hybridising nucleic acid from said sample to a collection of probes as identified in table 3a, table 3b, table 3c, table 3d, table 4a, table 4b, table 4c, table 4d, table 6a, table 7a, table 10a, table 10b, table 11a, table 11b, table 12b, table 12c, table 15a, table 15b, table 16a, table 16b, table 18b, table 18c, table 19c, table 19d, table 20, table 21, table 25a, table 26, table 27, table 28a, table 28b, table 29a, table 29b, table 30, table 31a, table 31b, table 32a, table 32b, table 33, table 34 and/or table 35, quantifying hybridization of said probes with nucleic acid in said sample, and comparing the result of said quantification with a reference.

27. A method according to claim 25 or claim 26, wherein said reference is a quantification of a hybridization of a sample comprising nucleic acid from adenoma cells or from normal cells, with said collection of probes.

28. A method according to claim 24 or claim 27, wherein said normal cells are brush cells from normal colon.

29. Use of a collection of probes or primers for small RNAs, preferably miRNAs and/or precursors thereof for classifying a sample as derived from a cell that contains a chromosomal aberration.

30. Use according to claim 29, wherein said cells are tumor cells, preferably colon tumor cells.

31. Use according to claim 29 or claim 30, wherein said collection of probes or primers is a collection of probes or primers according to any one of claims 1-5, 12-15.

32. Use according to any one of claims 29-31, for detecting gain or loss of a chromosome arm or a substantial part thereof.

33. Use according to claim 32, for detecting loss of chromosome 8p or 17p.

34. A collection of probes comprising the nucleic acid sequences identified in table 21.

35. A subset of probes according to claim 34, comprising at least 10 of the probes identified in table 21.

36. A subset of probes according to claim 35, comprising the probes for the nucleic acids as identified in table 1, table 2a, table 2b, table 2c, table 2d, table 3a, table 3b, table 3c, table 3d, table 4a, table 4b, table 4c, table 4d, table 5a, table 6a, table 7a, table 9a, table 9b, table 10a, table 10b, table 11a, table 11b, table 12a, table 12b, table 12c, table 14a, table 14b, table 15a, table 15b, table 16a, table 16b, table 18a, table 18b, table 18c, table 19c, table 19d, table 20, table 25a, table 26, table 27, table 28a, table 28b, table 29a, table 29b, table 30, table 31a, table 31b, table 32a, table 32b, table 33, table 34 and/or table 35.

37. A subset of probes from the collection of probes according to claim 34, comprising the probes for the nucleic acids as identified in table 8 or table 13.

38. A method for detecting cells in a sample of blood or a fractionated part thereof, comprising detecting small RNA in said sample.

39. A method according to claim 38, wherein said cells are colon cells.

40. A method according to claim 38 or claim 39, comprising detecting the level of nucleic acid molecules identified in table 14a, table 14b, table 15a, table 15b, table 16a, table 16b, table 18a, table 18b, or table 18c, table 23, table 24 and/or table 35.

41. A method for detecting cells in a faeces sample comprising detecting small RNA in said sample.

42. A method according to claim 41, wherein said cells are colon cells.

43. A method according to claim 41 or claim 42, comprising detecting the level of nucleic acid molecules identified in table 9a, table 9b, table 10a, table 10b, table 11a, table 11b, table 12a, table 12b, table 12c, table 23, table 24 and/or table 34.

44. A method for the treatment and/or prevention of a disease in an individual comprising administering to said individual at least one nucleic acid molecule of table 23 or table 24, at least one nucleic acid molecule comprising the reverse complementary sequence of a nucleic acid molecule of table 23 or table 24, or a functional equivalent of said at least one nucleic acid molecule.

45. A method according to claim 44, wherein said individual is suffering from colon cancer or is at risk of suffering therefrom.

46. A method according to claim 44 or claim 45, wherein said at least one nucleic acid molecule of table 23 or table 24 or the reverse complement thereof, is a nucleic acid molecule of table 2a, table 3a, table 4a, table 5a, table 6a, table 7a, table 9a, table 9b, table 10a, table 10b, table 11a, table 11b, table 12a, table 12b, table 12c, table 14a, table 14b, table 15a, table 15b, table 16a, table 16b, table 19c, table 19d, table 20, table 25a, table 26, table 27, table 28a, table 28b, table 29a, table 29b, table 30, table 31a, table 31b, table 32a, table 32b, table 33, table 34 and/or table 35, or a functional equivalent thereof.

47. A method according to any one of claims 44-46, wherein said at least one nucleic acid molecule of table 23 or table 24 or the reverse complement thereof, is a nucleic acid molecule of table 2a, table 2b, table 2c, table 2d, table 3a, table 3b, table 3c, table 3d, table 4a, table 4b, table 4c, table 4d, table 5a, table 6a, table 7a, table 9a, table 9b, table 10a, table 10b, table 11a, table 11b, table 12a, table 12b, table 12c, table 14a, table 14b, table 15a, table 15b, table 16a, table 16b, table 19c, table 19d, table 20, table 25a, table 26, table 27, table 28a, table 28b, table 29a, table 29b, table 30, table 31a, table 31b, table 32a, table 32b, table 33, table 34 and/or table 35, or a functional equivalent thereof.

48. A method according to any one of claims 44-47, wherein said at least one nucleic acid molecule of table 23 or table 24 or the reverse complement thereof, is a nucleic acid molecule of table 20, table 26 or table 27.

49. A method according to any one of claims 44-48, for the treatment of disease, wherein said at least one nucleic acid molecule of table 23 or table 24 or the reverse complement thereof, is a nucleic acid molecule of table 3a, table 4a, table 6a, table 7a, table 10a, table 10b, table 11a, table 11b, table 12b, table 12c, table 15a, table 15b, table 16a, table 16b, table 18b, table 18c, table 19c, table 19d, table 20, table 25a, table 26, table 27 or table 33, or a functional equivalent thereof.

50. A method according to any one of claims 44-49, for the treatment of disease, wherein said at least one nucleic acid molecule of table 23 or table 24 or the reverse complement thereof, is a nucleic acid molecule of table 5a, table 6a, table 7a or table 25a, or a functional equivalent thereof.

51. A method according to any one of claims 44-48, for the prevention of disease, wherein said at least one nucleic acid molecule of table 23 or table 24 or the reverse complement thereof, is a nucleic acid molecule of table 2a, table 4a, table 5a, table 7a, table 9a, table 9b, table 11a, table 11b, table 12a, table 12c, table 14a, table 14b, table 16a, table 16b, table 18a, table 18c, table 19c, table 19d, table 20, table 25a, table 26 or table 27, or a functional equivalent thereof.

52. A method according to any one of claims 44-48, 51, for the prevention of disease, wherein said at least one nucleic acid molecule of table 23 or table 24 or the reverse complement thereof, comprises a nucleic acid molecule of table 5a, table 7a or table 25a, or a functional equivalent thereof.

53. A method according to any one of claims 44-52, wherein said at least one nucleic acid molecule of table 23 or table 24, comprises a nucleic acid molecule of table 2d, table 3d, table 4d, table 19c, table 19d, table 26, table 27 or table 33.

54. A method according to claim 53, wherein said at least one nucleic acid molecule of table 23 or table 24, comprises a nucleic acid molecule of table 5a, table 7a, or table 25a or a functional equivalent thereof.

55. A method according to any one of claims 44-53, wherein said at least one nucleic acid molecule comprising the reverse complementary sequence of a nucleic acid molecule of table 23 or table 24, comprises a nucleic acid molecule of table 2c, table 3c, table 4c or table 19c or table 19d.

56. A method according to claim 55, wherein said at least one nucleic acid molecule of table 23 or table 24, comprises a nucleic acid molecule of table 5a, table 7a, or table 25a or a functional equivalent thereof.

57. Use of a nucleic acid molecule of table 23 or table 24, as a drug target.

58. Use according to claim 58, wherein said nucleic acid molecule is a nucleic acid molecule as identified in table 20, table 26 or table 27.

59. Use of a nucleic acid molecule of table 23 or table 24, for identifying a molecular process in a cell that is contributing to a disease or a disease state.

60. Use according to claim 59, wherein said nucleic acid molecule is a nucleic acid molecule as identified in table 20, table 26 or table 27.

61. A collection of nucleic acid molecules comprising the nucleic acid molecules of table 23 or table 24.

62. A subset nucleic acid molecules of a collection of nucleic acids according to claim 61, comprising at least 10 of the nucleic acids identified in table 23 or table 24.

63. A subset of nucleic acid molecules according to claim 62, comprising the nucleic acid molecules identified in table 1, table 2a, table 3a, table 4a, table 5a, table 6a, table 7a, table 9a, table 9b, table 10a, table 10b, table 11a, table 11b, table 12a, table 12b, table 12c, table 14a, table 14b, table 15a, table 15b, table 16a, table 16b, table 18a, table 18b, table 18c, table 19c, table 19d table 20, table 25a, table 26, table 27, table 28a, table 28b, table 29a, table 29b, table 30, table 31a, table 31b, table 32a, table 32b, table 33, table 34 and/or table 35.

64. A subset of nucleic acid molecules according to claim 62, comprising the nucleic acid molecules identified in table 8 or table 13.

65. A method for determining whether a colon cell comprises a deletion of less than 3 megabases in chromosomal region 1p32.2, 1q21.1, 1q22, 6p22.2, 7p15.2, 11p15.5, 16q12.2 or 17p13.1 comprising determining whether said cell comprises a deletion of DNA coding for a small RNA as identified in table 28a, or a precursor thereof.

66. A method according to claim 65, wherein nucleic acid of said cell is contacted with a primer or a probe specific for said small RNA, a precursor thereof, a DNA encoding said small RNA and/or a precursor thereof.

67. A method according to claim 65 or claim 66, wherein said small RNA is a small RNA as identified in table 28b.

68. A method according to any one of claims 65-67, further comprising typing said cell as a colon carcinoma cell.

69. A method according to any one of claims 65-68, wherein said small RNA is a small RNA as identified in table 30.

70. A method according to claim 69, further comprising typing said cell as a microsatellite stable colon carcinoma cell.

71. A method according to any one of claims 65-68, wherein said small RNA is a small RNA as identified in table 32a or table 32b.

72. A method according to claim 70, wherein said small RNA is a small RNA as identified in table 32b.

73. A method according to claim 70 or claim 71, further comprising typing said cell as a microsatellite instable colon carcinoma cell.

74. A method for determining whether a colon cell comprises an additional chromosomal copy of less than 3 megabases of chromosomal region 5q31.3, 8p21.3, 9p21.3, 10p15.1 or 16q13 comprising determining whether said cell comprises an additional copy of DNA coding for a small RNA as identified in table 29a, or a precursor thereof.

75. A method according to claim 74, wherein nucleic acid of said cell is contacted with a primer or a probe specific for said small RNA, a precursor thereof, a DNA encoding said small RNA and/or a precursor thereof.

76. A method according to claim 74 or claim 75, wherein said small RNA is a small RNA as identified in table 29b.

77. A method according to any one of claims 74-76, further comprising typing said cell as a colon carcinoma cell.

78. A method according to any one of claims 74-77, wherein said small RNA is a small RNA as identified in table 31a or table 31b.

79. A method according to claim 78, wherein said small RNA is a small RNA as identified in table 31b.

80. A method according to claim 78 or claim 79, further comprising typing said cell as a microsatellite stable colon carcinoma cell.

81. A method for the treatment of an individual suffering from a colon tumor comprising administering to said individual a nucleic acid comprising a nucleic acid as identified in table 28a, table 28b, table 30, table 32a, table 32b or 33 or a functional equivalent thereof.

82. A method according to claim 81, wherein said nucleic acid is a nucleic acid as identified in table 28b, table 32b or table 33.

83. A method according to claim 81 or claim 82, wherein the nucleic acid is provided as a nucleic acid encoding a precursor for the nucleic acid.
